(19) United States
(12) Patent Application Publication
(10) Pub. No.: US 2023/0158155 A1
(43) Pub. Date: May 25, 2023

(54) Cell Surface Receptor Binding Compounds and Conjugates

(71) Applicant: Lycia Therapeutics, Inc., South San Francisco, CA (US)

(72) Inventors: Brett Bradley BUSCH, San Diego, CA (US); Justin Thomas ERNST, San Diego, CA (US); Garrick K. PACKARD, San Diego, CA (US); Jason G. LEWIS, Castro Valley, CA (US); Eric D. TURTLE, Belmont, CA (US)

(21) Appl. No.: 17/792,027

(22) PCT Filed: Jan. 8, 2021
(86) PCT No.: PCT/US21/12846

§ 371 Date: Jul. 11, 2022

Related U.S. Application Data
(60) Provisional application No. 62/959,862, filed on Jan. 10, 2020.
(60) Provisional application No. 62/959,877, filed on Jan. 10, 2020.
(60) Provisional application No. 62/959,882, filed on Jan. 10, 2020.
(60) Provisional application No. 63/043,752, filed on Jun. 24, 2020.
(60) Provisional application No. 63/043,754, filed on Jun. 24, 2020.
(60) Provisional application No. 63/043,749, filed on Jun. 24, 2020.

(51) Int. Cl. A61K 47/54; A61K 47/68; A61P 1/16; A61P 35/00

(52) U.S. Cl. CPC A61K 47/549; A61K 47/6849; A61P 1/16; A61P 35/00

(57) ABSTRACT

The present disclosure provides a class of compounds including a ligand moiety that specifically binds to a cell surface receptor, such as a mannose-6-phosphate receptor (M6PR) or a cell surface asialoglycoprotein receptor (ASGPR). The cell surface M6PR or ASGPR binding compounds can trigger the receptor to internalize into the cell abound compound. The ligand moieties of this disclosure can be linked to a variety of moieties of interest without impacting the specific binding to, and function of, the cell surface receptor, e.g., M6PR or ASGPR. Also provided are compounds that are conjugates of the ligand moieties linked to a biomolecule, such as an antibody, which conjugates can harness cellular pathways to remove specific proteins of interest from the cell surface or from the extracellular milieu. Also provided are methods of using the conjugates to target a polypeptide of interest for sequestration and/or lysosomal degradation.

### CROSS REFERENCE TO RELATED APPLICATIONS

This application claims the benefit of U.S. Application No. 62/959,877, filed Jan. 10, 2020, U.S. Application No. 62/959,862, filed Jan. 10, 2020, U.S. Application No. 62/959,882, filed Jan. 10, 2020, U.S. Application No. 63/043,749, filed Jun. 24, 2020, U.S. Application No. 63/043,752, filed Jun. 24, 2020, and U.S. Application No. 63/043,754, filed Jun. 24, 2020, which applications are incorporated herein by reference in their entirety.

### REFERENCE TO SEQUENCE LISTING SUBMITTED ELECTRONICALLY

This application incorporates by reference a Sequence Listing submitted with this application as text file entitled 47970WO Seqlist created on Jun. 17, 2020 and having a size of 15,951 bytes.

### INTRODUCTION

Many therapeutics act by binding a functionally important site on a target protein, thereby modulating the activity of that protein, or by recruiting immune effectors, as with many monoclonal antibody drugs, to act upon the target protein. However, there is an untapped reservoir of medically important human proteins that are considered to be “undruggable” because these proteins are not readily amenable to currently available therapeutic targeting approaches. Thus, there is a need for therapies that can target a wider range of proteins.

Mannose-6-phosphate is a monosaccharide ligand that plays a key role in the intracellular retention and secretion of lysosomal hydrolytic enzymes to which they are attached. When this sugar residue is incorporated onto newly synthesized enzymes it can direct their transport from the Golgi apparatus to the lysosomes where they are active. Membrane-bound, cell surface mannose-6-phosphate receptors (M6PR's) play a role in many biological processes, including the secretion and internalization of such lysosomal enzymes. Endocytosis by an M6PR allows for the internalization into the cell of compounds bearing a mannose 6-phosphate (M6P) ligand and trafficking to lysosomes.

Alternative ligands that provide for binding to cell surface M6PRs followed by transport across cell membranes are of great interest.

### SUMMARY

The present disclosure provides a class of compounds including a ligand moiety that specifically binds to a cell surface receptor. In some embodiments, the ligand moiety binds to a mannose-6-phosphate receptor (M6PR). In some embodiments, the ligand moiety binds to a cell surface asialoglycoprotein receptor (ASGPR). The cell surface M6PR or ASGPR binding compounds can trigger the receptor to internalize into the cell a bound compound. The ligand moieties of this disclosure can be linked to a variety of moieties of interest without impacting the specific binding to, and function of, the cell surface receptor, e.g., M6PR or ASGPR. Also provided are compounds that are conjugates of the ligand moieties linked to a biomolecule, such as an antibody, which conjugates can harness cellular pathways to remove specific proteins of interest from the cell surface or from the extracellular milieu. For example, the conjugates described herein may sequester and/or degrade a target molecule of interest in a cell's lysosome. Also provided herein are compositions comprising such conjugates and methods of using the conjugates to target a polypeptide of interest for sequestration and/or lysosomal degradation, and methods of using the conjugates to treat disorders or disease.

A first aspect of this disclosure includes a cell surface mannose-6-phosphate receptor (M6PR) binding compound of formula (XI):

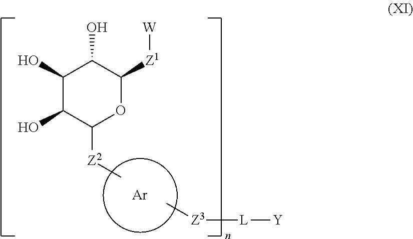

or a salt thereof, wherein:

each W is independently a hydrophilic head group;

each Z¹ is independently selected from optionally substituted (C₁-C₃)alkylene and optionally substituted ethenylene;

each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;

each Ar is independently an optionally substituted aryl or heteroaryl linking moiety (e.g., monocyclic or bicyclic aryl or heteroaryl, optionally substituted);

each Z³ is independently a linking moiety;

n is 1 to 500;

L is a linker; and

Y is a moiety of interest.

A second aspect of this disclosure includes a cell surface receptor binding conjugate of formula (I):

Xₙ-L-Y (I)

or a salt thereof,
wherein:

X is a moiety that binds to a cell surface asialoglycoprotein receptor (ASGPR) or a moiety that binds to a cell surface mannose-6-phosphate receptor (M6PR);

n is 1 to 500 (e.g., n is 1 to 20, 1 to 10, 1 to 6 or 1 to 5); and

L is a linker;

Y is a biomolecule that specifically binds a target protein.

In some embodiments of formula (I), Y is antibody or antibody fragment that specifically binds the target protein and the compound is of formula (V):

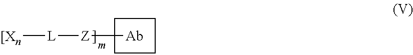

or a pharmaceutically acceptable salt thereof,
wherein:

n is 1 to 20;

m is an average loading of 1 to 80;

Ab is the antibody or antibody fragment that specifically binds the target protein; and

Z is a residual moiety resulting from the covalent linkage of a chemoselective ligation group to a compatible group of Ab.

A third aspect of this disclosure includes a method of internalizing a target protein in a cell comprising a cell surface receptor selected from M6PR and ASGPR, where the method includes contacting a cellular sample comprising the cell and the target protein with an effective amount of a compound or conjugate (e.g., as described herein) that specifically binds the target protein and specifically binds the cell surface receptor to facilitate cellular uptake of the target protein.

A fourth aspect of this disclosure includes a method of reducing levels of a target protein in a biological system, where the method includes contacting the biological system with an effective amount of a compound or conjugate (e.g., as described herein) that specifically binds the target protein and specifically binds a cell surface receptor of cells in the biological system to facilitate cellular uptake and degradation of the target protein.

### BRIEF DESCRIPTION OF THE DRAWINGS

FIG. 1: Representative SEC chromatogram of matuzumab-(Compound A) conjugate.

FIG. 2: Native Mass Spectrometry MS analysis of deglycosylated matuzumab and matuzumab-(Compound A) conjugate.

FIG. 3: Representative SEC chromatogram of matuzumab-(Compound I-7) conjugate.

FIG. 4: Native MS analysis of deglycosylated matuzumab and matuzumab-(Compound I-7) conjugate.

FIG. 5: Representative SEC chromatogram of atezolizumab-(Compound A) conjugate.

FIG. 6: Native MS analysis of deglycosylated atezolizumab and atezolizumab-(Compound A) conjugate.

FIG. 7: Representative SEC chromatogram of cetuximab-(Compound A) conjugate.

FIG. 8: Native MS analysis of deglycosylated cetuximab and cetuximab-(Compound A) conjugate.

FIG. 9: Representative SEC chromatogram of cetuximab-(Compound I-7) conjugate.

FIG. 10: Native MS analysis of deglycosylated cetuximab and cetuximab-(Compound I-7) conjugate.

FIG. 11: Representative SEC chromatogram of anti-PD-L1 antibody (29E.2A3)-(Compound A) conjugate.

FIG. 12: Native MS analysis of deglycosylated anti-PD-L1 antibody (29E.2A3) and anti-PD-L1 antibody (29E.2A3)-(Compound A) conjugate.

FIG. 13: Representative SEC chromatogram of IgG2a-UNLB-(Compound I-7) conjugate.

FIG. 14: Native MS analysis of deglycosylated IgG2a-UNLB and IgG2a-UNLB-(Compound I-7) conjugate.

FIG. 15: Time course activity of cetuximab-(Compound A) and cetuximab-(Compound I-7) conjugates on surface EGFR levels in Hela parental and M6PR KO cells measured by surface staining.

FIG. 16: Time course activity of matuzumab-(Compound A) and matuzumab-(Compound I-7) conjugates on surface EGFR levels in Hela parental and M6PR KO cells measured by surface staining.

FIG. 17: Dose response of cetuximab-(Compound A), cetuximab-(Compound I-7), matuzumab-(Compound A), and matuzumab-(Compound I-7) conjugates on total EGFR levels in Hela parental and M6PR KO cells measured by in-cell Western blotting.

FIG. 18: Time course activity of cetuximab-(Compound A), cetuximab-(Compound I-7), matuzumab-(Compound A), and matuzumab-(Compound I-7) conjugates on relative EGFR normalized levels in Hela parental and M6PR KO cells.

FIGS. 19A-19F: Binding affinities for M6PR of matuzumab conjugated to unlabeled control (FIG. 19A), Compound I-7 (FIG. 19B), Compound I-8 (FIG. 19C), Compound I-9 (FIG. 19D), compound I-11 (FIG. 19E) and Compound I-12 (FIG. 19F) to M6PR. Binding to M6PR was determined by ELISA. Compound I-7 (dar8) and Compound I-11 (dar4) showed the highest and lowest binding affinity, respectively. RFU: Relative fluorescence units.

FIGS. 20A-20C: Serum PK Analysis for Individual rIgG1 Antibody Conjugates. Intracellular levels of algG2a conjugated to Compound I-7 (dar8) and (dar4) (FIG. 20A), algG2a conjugated to Compound I-11 and algG2a conjugated to Compound I-11 (FIG. 20B), and algG2a conjugated to Compound I-9 and algG2a conjugated to Compound I-12 (FIG. 20C) in mouse serum were measured at 0.5, 1, 2, 6, and 24 hours using ELISA.

FIG. 21: Intracellular uptake of anti-IgG2a conjugates overtime in Jurkat cells. Conjugates were detected using Alex488-conjugated antibodies, and intracellular levels of fluorescence were determined by FACS after 1 hr and 24 hr.

FIG. 22: Intracellular uptake of anti-IgG2a conjugates into Jurkat cells at 10 nM after 24 hr as a percentage of the uptake of algG2a conjugate Compound I-7 (dar8).

FIG. 23: A graph of results of a M6PR binding assay for a variety of antibody conjugates of exemplary compounds with various DAR loadings.

FIG. 24: A graph of cell fluorescence versus antibody conjugate concentration indicating that various antibody conjugates of exemplary M6PR binding compounds exhibited robust uptake into Jurkat cells after one hour incubation.

FIG. 25: A graph of cell fluorescence versus antibody conjugate concentration indicating that various antibody conjugates of exemplary M6PR or ASGPR binding compounds exhibited robust uptake into HepG2 cells after one hour incubation.

FIG. 26: A graph demonstrating CI-M6PR dependent cell uptake of exemplary antibody conjugates in wild type (WT) K562 cells versus CI-M6PR knockout (KO) cells.

### DETAILED DESCRIPTION

As summarized above, this disclosure provides classes of compounds including a ligand moiety that specifically binds to a cell surface receptor. Also provided herein are conjugates that comprise a moiety, X, that binds to such a cell surface receptor, for example, an internalizing cell surface receptor, for example, for sequestration and/orlysosomal degradation. In certain embodiments, the cell surface receptor is a mannose-6-phosphate receptor (M6PR). In certain embodiments, the cell surface receptor is a asialoglycoprotein receptor (ASGPR).

This disclosure includes compounds of formula (I):

Xₙ-L-Y (I)

or a salt thereof, wherein:

X is a moiety that binds to a cell surface receptor selected from M6PR and ASGPR (e.g., as described herein);

n is 1 to 500;

L is a linker (e.g., monovalent or multivalent, as described herein) of defined length;

and

Y is a moiety of interest (e.g., as described herein).

The compounds and conjugates and methods of this disclosure are described in greater detail below. A particular class of M6PR binding compounds is described. Also described are biomolecule conjugates that include a cell surface receptor binding moiety (X) that binds to M6PR or to ASGPR. Linkers (L) and moieties of interest (Y) which find use in the M6PR binding compounds, and the biomolecule conjugates are also described. Methods in which the compounds and conjugates of this disclosure find use are also described.

### M6PR Binding Compounds

As summarized above, this disclosure provides a class of compounds including a ligand moiety that specifically binds to a cell surface mannose-6-phosphate receptor (M6PR). The M6PR ligand moieties of this disclosure can be linked to a variety of moieties of interest without impacting the specific binding to, and function of, the cell surface M6PR. The inventors have demonstrated that compounds of this disclosure can utilize the functions of cell surface M6PRs in a biological system, e.g., for internalization and sequestration of a compound to the lysosome of a cell, and in some cases subsequent lysosomal degradation. The compounds of this disclosure find use in a variety of applications.

The compounds of this disclosure can specifically bind to a cell surface M6PR, for example, an internalizing M6PR cell surface receptor. In particular embodiments, the surface M6PR is a human M6PR. In particular embodiments, the M6PR is Homo sapiens insulin like growth factor 2 receptor (IGF2R) (see, e.g., NCBI Reference Sequence: NM_000876.3), also referred to as cation-independent mannose-6-phosphate receptor (CI-MPR). MPGR endogenously transports proteins bearing N-glycans capped with mannose phosphate (M6P) residues to lysosomes, and cycles between endosomes, the cell surface, and the Golgi complex. See, e.g., Ghosh et al., Nat. Rev. Mol. Cell Biol. 2003; 4: 202-213.

The M6PR binding compounds of this disclosure include a moiety (X) that specifically binds to the cell surface receptor M6PR. For example, a mannose-6-phosphate (M6P) or an M6P analog or derivative (e.g., as described herein), that specifically binds to a cell surface M6PR. The M6PR binding compounds can be monovalent or multivalent (e.g., bivalent or trivalent or of higher valency), where a monovalent compound includes a single M6PR ligand moiety, and a monovalent compound includes two or more such moieties.

A compound comprising such X (e.g., as described herein), may bind to other receptors, for example, may bind with lower affinity as determined by, e.g., immunoassays or other assays known in the art. In a specific embodiment, X, or a compound as described herein comprising such X specifically binds to the cell surface M6PR with an affinity that is at least 2 logs, 2.5 logs, 3 logs, 4 logs or greater than the affinity when X or the compound or the conjugate bind to another cell surface receptor. In a specific embodiment, X, e.g., M6P or an M6P analog or derivative, or a compound as described herein comprising X, specifically binds to M6PR with an affinity (K_{d}) less than or equal to 20 mM. In particular embodiments, such binding is with an affinity (K_{d}) less than or equal to about 20 mM, about 10 mM, about 1 mM, about 100 uM, about 10 uM, about 1 uM, about 100 nM, about 10 nM, or less than or equal to about 1 nM. Unless otherwise noted, “binds,” “binds to,” “specifically binds” or “specifically binds to” in this context are used interchangeably.

In certain embodiments, the M6PR binding moiety X is able to bind to a M6PR specific cell surface receptor, and direct (or target) the molecule to this receptor. In certain embodiments, M6PR binding moiety X is capable of binding to the M6PR and directing (or targeting) a compound or conjugate described herein for internalization and sequestration to the lysosome, and/or subsequent lysosomal degradation.

In some embodiments, the M6PR binding moiety X includes a mannose sugar ring, or analog thereof, with a hydrophilic head group that is linked via a linking moiety to the 5-position of the ring. The linking moiety can be of 1-6 atoms in length, such as 1-5, 1-4 or 1-3 atoms in length. The hydrophilic head group can be any convenient group that is charged or readily capable of hydrogen bonding or electrostatic interactions under aqueous or physiological conditions. The hydrophilic head group can be a structural or functional mimic of the 6-phosphate group of M6P that has desirable stability. The hydrophilic head group can have a MW of less than 200, such as less than 150 or less than 100. In some embodiments, the hydrophilic head group is a phosphonate. In some embodiments, the hydrophilic head group is a thiophosphonate. In some embodiments, the hydrophilic head group is a phosphate, thiophosphate or dithiophosphate.

In some embodiments, the mannose sugar ring of X is linked to an optionally substituted aryl or heteroaryl group that together provide a moiety having a desirable binding affinity and activity at the M6P receptor of interest. Multiple M6PR binding moieties X can be linked together to provide multivalent binding to the M6PR. The M6PR binding moiety or moieties X can be further linked to any convenient moiety or molecule of interest (e.g., as described herein).

Accordingly, provided herein are M6PR binding compounds of formula (Ia):

Xₙ-L-Y (Ia)

or a salt thereof,
wherein:

X is a moiety that binds to a cell surface M6PR (e.g., M6PR ligand or binding moiety, e.g., as described herein);

n is 1 to 500;

L is a linker of defined length; and

Y is a moiety of interest.

The M6PR binding moiety (X) of the compounds of this disclosure can include a mannose ring or analog thereof described by the following structure:

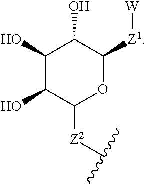

where:

W is a hydrophilic head group;

Z¹ is selected from optionally substituted (C₁-C₃)alkylene and optionally substituted ethenylene;

Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

The mannose ring or analog thereof of the M6PR binding moiety can be incorporated into the compounds of this disclosure by attachment to the Z² group via a linking moiety. It is understood that in the compounds of formula (Ia), the group or linking moiety attached to Z² can, in some cases, be considered to be part of the M6PR binding moiety (X) and provide for desirable binding to the M6PR. In certain other cases, the group or linking moiety attached to Z² can be considered part of the linker L of formula (Ia).

In one aspect, provided herein are cell surface mannose-6-phosphate receptor (M6PR) binding compounds of formula (XI):

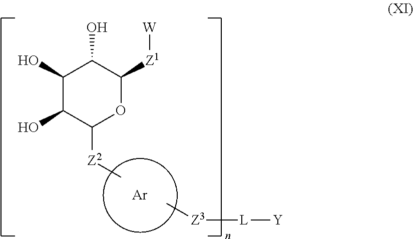

or a salt thereof,
wherein:

each W is independently a hydrophilic head group;

each Z¹ is independently selected from optionally substituted (C₁-C₃)alkylene and optionally substituted ethenylene;

each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;

each Ar is independently an optionally substituted aryl or heteroaryl group or linking moiety;

each Z³ is independently a linking moiety;

n is 1 to 500;

L is a linker; and

Y is a moiety of interest.

In some embodiments of formula (XI), when n is 1 and Ar is phenyl, then: i) L comprises a backbone of at least 16 consecutive atoms (e.g., at least 20 consecutive atoms, in some cases up to about 200 consecutive atoms); ii) Y is a biomolecule; and/or ii) Z³ is amide, sulfonamide, urea or thiourea linking moiety.

The Ar group linking moiety of formula (XI) can be a monocyclic aryl or monocyclic heteroaryl group. In some embodiments of formula (XI), Ar is a 5-membered monocyclic heteroaryl group. In some embodiments of formula (XI), Ar is a 6-membered monocyclic aryl or heteroaryl group. The Ar group linking moiety of formula (XI) can be a multicyclic aryl or multicyclic heteroaryl group, such as a bicyclic aryl or bicyclic heteroaryl group. In some embodiments of formula (XI), Ar is a fused bicyclic group. In some embodiments of formula (XI), Ar is a bicyclic group comprising two aryl and/or heteroaryl monocyclic rings connected via a covalent bond. In some embodiments of formula (XI), Ar is a bicyclic aryl or bicyclic heteroaryl group having two 6-membered rings. In some embodiments of formula (XI), Ar is a bicyclic aryl or bicyclic heteroaryl group having one 6-membered ring that is connected via a covalent bond or fused to a 5-membered ring.

In some embodiments of formula (XI), each Ar is independently selected from optionally substituted phenyl, optionally substituted pyridyl, optionally substituted biphenyl, optionally substituted naphthalene, optionally substituted quinoline, optionally substituted triazole and optionally substituted phenylene-triazole. In some embodiments of formula (XI), Ar is substituted with at least one OH substituent. In some embodiments of formula (XI), Ar is substituted with 1, 2, or more OH groups. In some embodiments of formula (XI), Ar is substituted with at least one optionally substituted (C₁-C₆)alkyl.

In some embodiments of formula (XI), Ar is optionally substituted 1,4-phenylene, optionally substituted 1,3-phenylene, or optionally substituted 2,5-pyridylene.

In some embodiments of formula (XI), the compound is of formula (XIIa) or (XIIb):

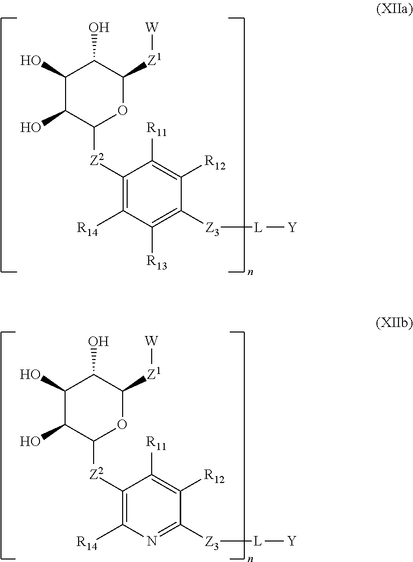

or a salt thereof,
wherein:

each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each R²⁵ is independently selected from H, and optionally substituted (C₁-C₆)alkyl.

In some embodiments of formula (XIIa)-(XIIb), R¹¹ to R¹⁴ are each H. In some embodiments of formula (XIIa)-(XIIb), at least one of R¹¹ to R¹⁴ is OH, such as 1, 2, or more of R¹¹ to R¹⁴ is OH.

In some embodiments of formula (XIIa)-(XIIb), Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —NR²³SO₂—, and —SO₂NR²³—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (XI)-(XIIb), Z³ is a covalent bond to L.

In some embodiments of formula (XI)-(XIIb), Z³ is optionally substituted amido, urea or thiourea. In some embodiments of formula (XI)-(XIIb), Z³ is

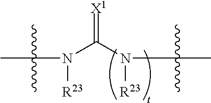

wherein:

X¹ is O or S;

t is 0 or 1; and

each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is an urea or thiourea.

In some embodiments of formula (XI)-(XIIb), Z³ is —N(R²³)SO₂— or —SO₂N(R²³)—.

In some embodiments of formula (XI)-(XIIb), Z³ is —N(R²³)CO— or —CON(R²³)—.

In some embodiments of formula (XI)-(XIIb), Z³ is —NHC(═X¹)NH—, wherein X¹ is O or S. In some embodiments, X¹ is O. In some embodiments, X¹ is S.

In some embodiments of formula (XI)-(XIIb), —Ar—Z³— is selected from:

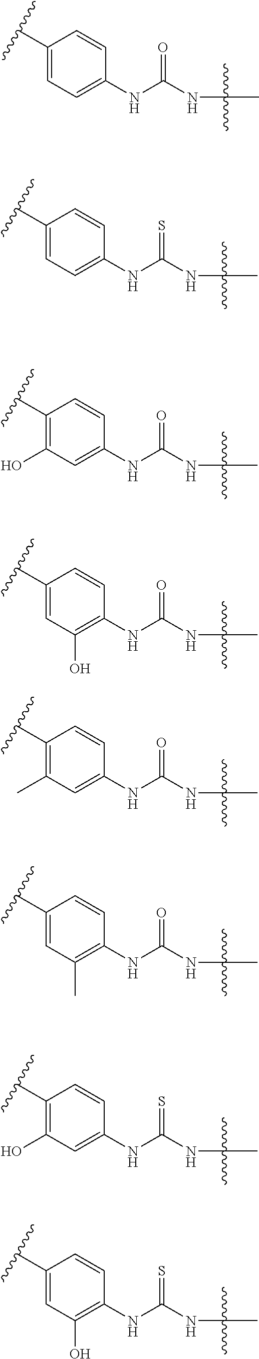

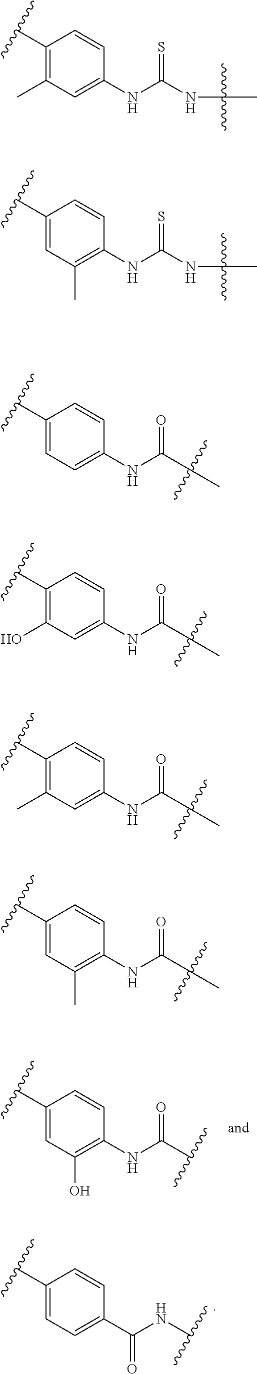

In some embodiments of formula (XI)-(XIIb), Z³ is optionally substituted triazole. When Z³ is optionally substituted triazole, it can be synthetically derived from click chemistry conjugation of an azido containing precursor and an alkyne containing precursor of the compound. Accordingly, in some embodiments of formula (XIIa)-(XIIb), the compound is of formula (XIIc) or (XIId):

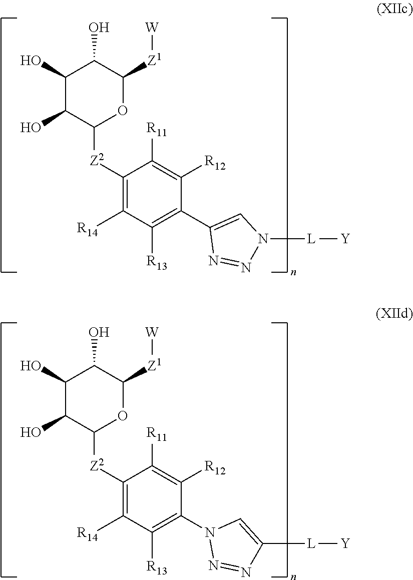

or a salt thereof,
wherein:

each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each R²⁵ is independently selected from H, and optionally substituted (C₁-C₆)alkyl.

In some embodiments of formula (XIIc)-(XIId), R¹¹ to R¹⁴ are each H. In some embodiments of formula (XIIc)-(XIId), at least one of R¹¹ to R¹⁴ is OH, such as 1, 2, or more of R¹¹ to R¹⁴ is OH.

In some embodiments of formula (XIIc)-(XIId), —Ar—Z³— is selected from:

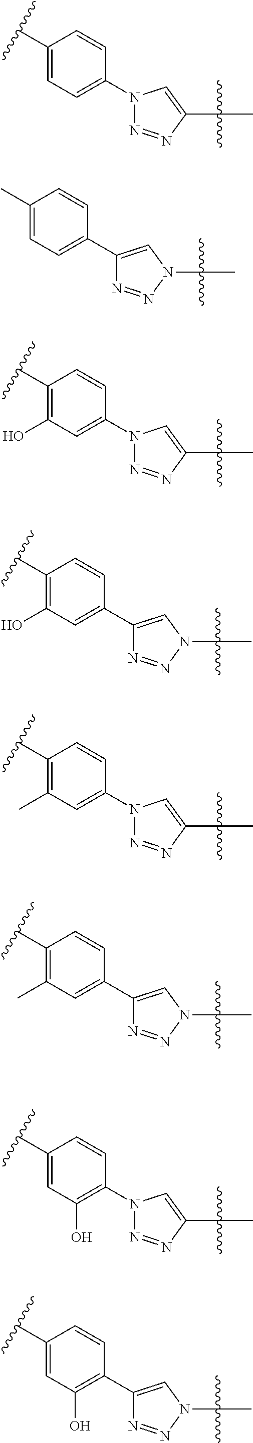

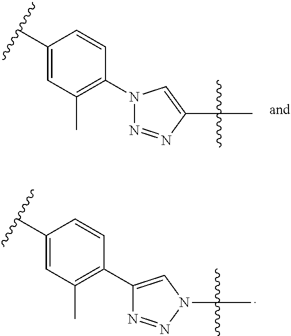 [SMILES: *c1ccc(-n2cc(*)nn2)c(C)c1 ; *c1ccc(-c2cn(*)nn2)c(C)c1]

In some embodiments of formula (XI), Ar is an optionally substituted fused bicyclic aryl or heteroaryl. In some embodiments of formula (XI), Ar is optionally substituted naphthalene or optionally substituted quinoline. In some embodiments of formula (XI), the compound is of formula (XIIIa), (XIIIb) or (XIIIb′):

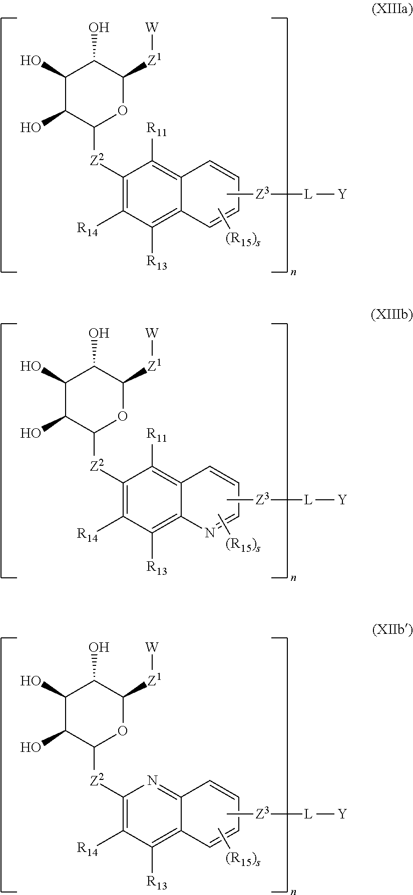

or a salt thereof, wherein:

each R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;

s is 0 to 3; and

each R²⁵ is independently selected from H, and optionally substituted (C₁-C₆)alkyl.

In some embodiments of formula (XIIIa)-(XIIIb′), the compound is of formula (XIIIc)-(XIIIh):

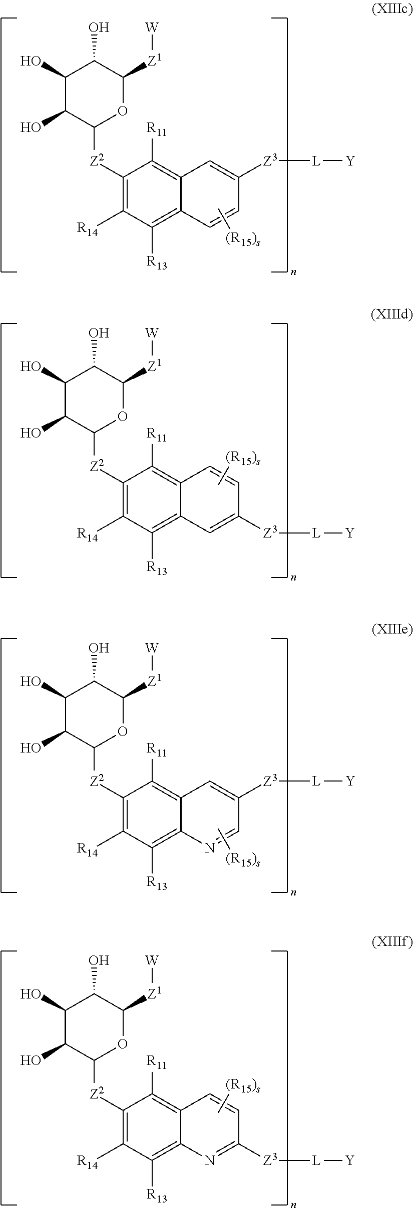

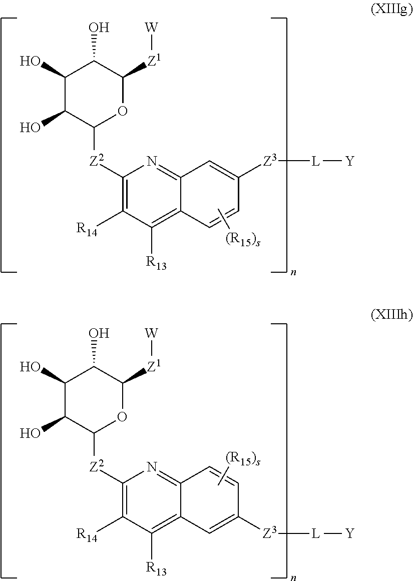

or a salt thereof.

In some embodiments of formula (XIIIa)-(XIIIh), R¹¹ to R¹⁴ are each H and s is O. In some embodiments of formula (XIIIa)-(XIIIh), at least one of R¹¹ to R¹⁵ is OH, such as 1, 2, or more of R¹¹ to R¹⁵ is OH.

In some embodiments of formula (XIIIa)-(XIIIh), Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (XIIIa)-(XIIIh), Z³ is a covalent bond to L.

In some embodiments of formula (XIIIa)-(XIIIh), Z³ is optionally substituted amido, urea or thiourea. In some embodiments of formula (XIIIa)-(XIIIh), Z³ is

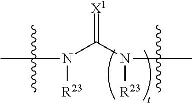

wherein:

X¹ is O or S;

t is 0 or 1; and

each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is an urea or thiourea.

In some embodiments of formula (XIIIa)-(XIIIh), Z³ is —N(R²³)SO₂— or —SO₂N(R²³)—.

In some embodiments of formula (XIIIa)-(XIIIh), Z³ is —N(R²³)CO— or —CON(R²³)—.

In some embodiments of formula (XIIIa)-(XIIIh), Z³ is —NHC(═X¹)NH—, wherein X¹ is O or S. In some embodiments, X¹ is O. In some embodiments, X¹ is S.

In some embodiments of formula (XIIIa)-(XIIIh), Z³ is optionally substituted triazole. When Z³ is optionally substituted triazole, it can synthetically derived from click chemistry conjugation of an azido containing precursor and an alkyne containing precursor of the compound.

In some embodiments of formula (XIIIa)-(XIIIh), —Ar—Z³— is selected from:

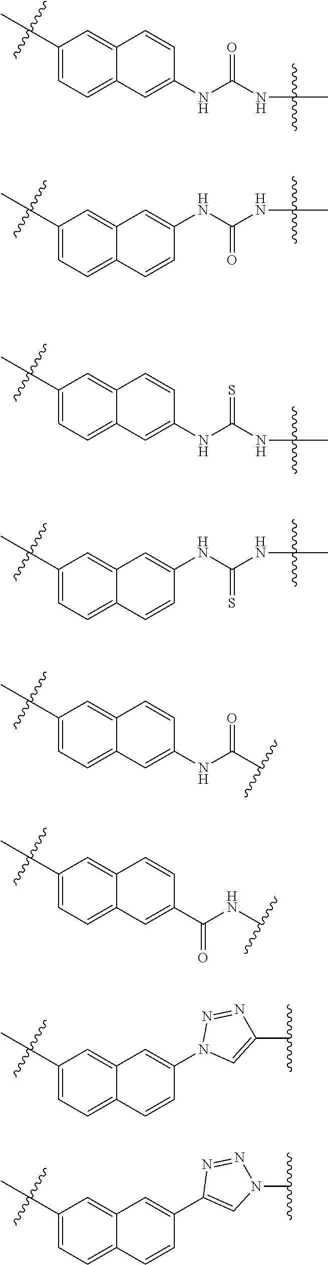

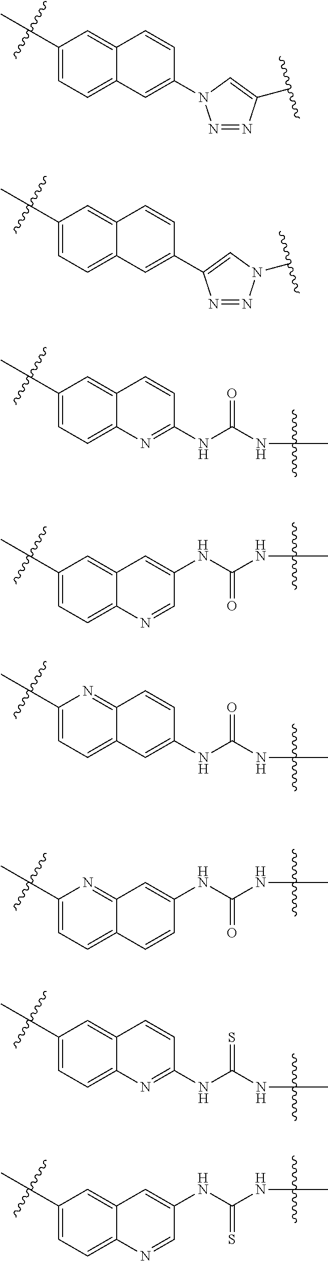

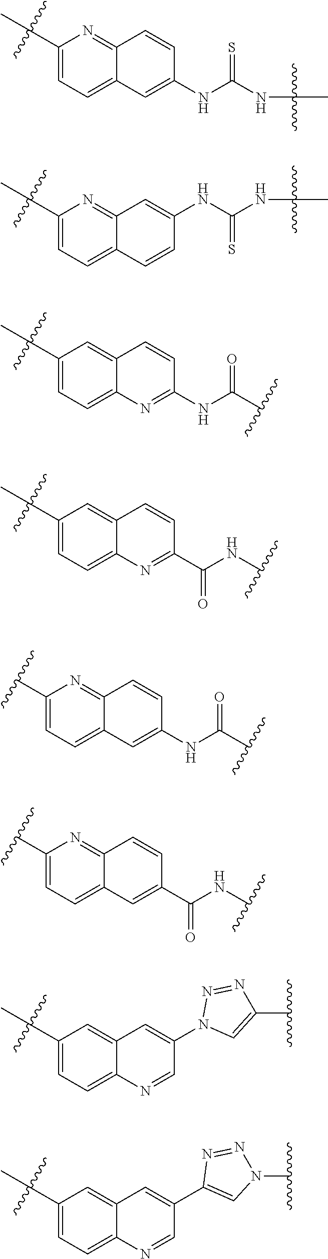

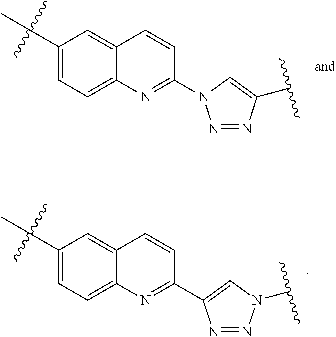 [SMILES: *c1ccc2nc(-n3cc(*)nn3)ccc2c1 ; *c1ccc2nc(-c3cn(*)nn3)ccc2c1]

In some embodiments of formula (XI), Ar is optionally substituted bicyclic aryl or optionally substituted bicyclic heteroaryl and wherein the compound is of formula (XIVa)

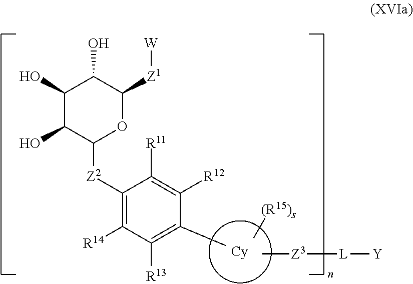

or a salt thereof,
wherein:

each Cy is independently monocyclic aryl or monocyclic heteroaryl;

each R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;

s is 0 to 4; and

each R²⁵ is independently selected from H, and optionally substituted (C₁-C₆)alkyl.

In some embodiments of formula (XIVa), Ar is optionally substituted biphenyl, Cy is optionally substituted phenyl, and the compound is of formula (XIVb):

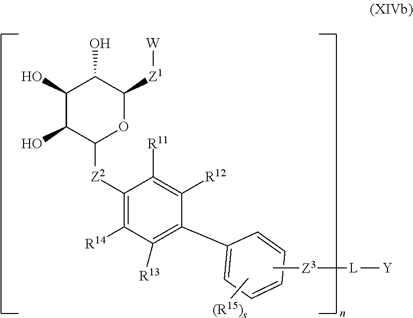

or a salt thereof.

In some embodiments of formula (XIVb), the compound is of formula (XIVc) or (XIVd):

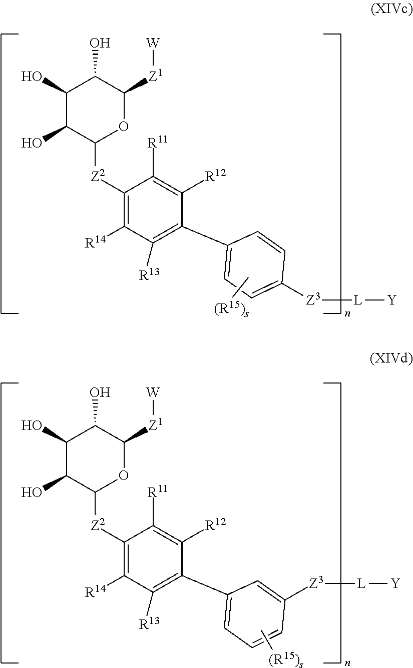

or a salt thereof.

In some embodiments of formula (XI)-(XIVd), Ar is substituted with at least one OH substituent. In some embodiments of formula (XI)-(XIVd), R¹¹ to R¹⁵ are each H. In some embodiments of formula (XI)-(XIVd), at least one of R¹¹ to R¹⁵ is OH, such as 1, 2, or more of R¹¹ to R¹⁵ is OH.

In some embodiments of formula (XI)-(XIVd), Z³ is selected from a covalent bond, —O—, —NR²³—, —NR²³CO—, —CONR²³—, —NR²³CO₂, —OCONR²³, —NR²³C(═X¹)NR²³—, —CR²⁴═N—, —CR²⁴═N—X², —N(R²³)SO₂— and —SO₂N(R²³)—; wherein X¹ and X² are selected from O, S and NR²³; and R²³ and R²⁴ are independently selected from H, C₍₁₋₃₎-alkyl (e.g., methyl) and substituted C₍₁₋₃₎-alkyl.

In some embodiments of formula (XI)-(XIVd), Z³ is a covalent bond to L.

In some embodiments of formula (XI)-(XIVd), Z³ is optionally substituted amido, urea or thiourea. In some embodiments of formula (XI)-(XIVd), Z³ is

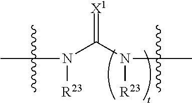

wherein:

X¹ is O or S;

t is 0 or 1; and

each R²³ is independently selected from H, C₍₁₋₃₎-alkyl (e.g., methyl or ethyl) and substituted C₍₁₋₃₎-alkyl. In some embodiments of Z³, X¹ is O. In some embodiments of Z³, X¹ is S. In some embodiments of Z³, t is 0 and X¹ is O, such that Z³ is amido. In some embodiments of Z³, t is 1 such that Z³ is an urea or thiourea.

In some embodiments of formula (XI)-(XIVd), Z³ is —NHC(═X¹)NH—, wherein X¹ is O or S. In some embodiments, X¹ is O. In some embodiments, X¹ is S.

In some embodiments of formula (XI)-(XIVd), Z³ is —N(R²³)SO₂— or —SO₂N(R²³)—.

In some embodiments of formula (XI)-(XIVd), Z³ is optionally substituted triazole. When Z³ is optionally substituted triazole, it can be synthetically derived from click chemistry conjugation of an azido containing precursor and an alkyne containing precursor of the compound.

In some embodiments of formula (XI)-(XIVd), —Ar—Z³— is selected from:

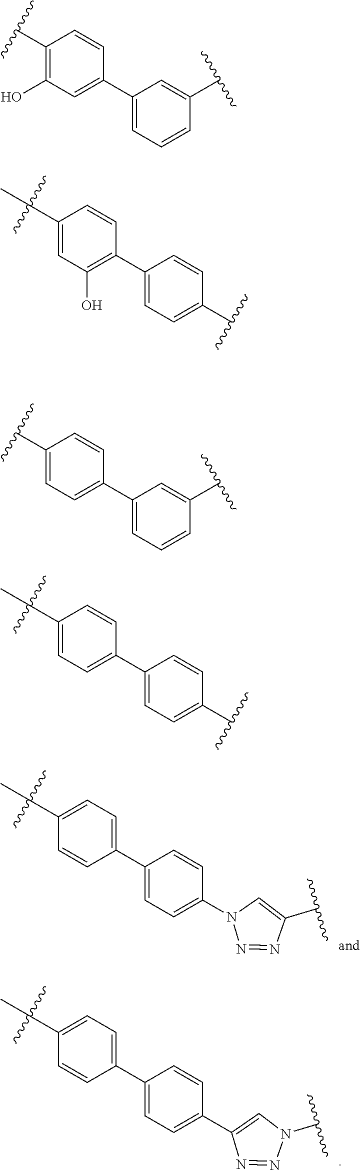 [SMILES: *c1cccc(-c2ccc(*)c(O)c2)c1 ; *c1ccc(-c2ccc(*)cc2O)cc1 ; *c1ccc(-c2cccc(*)c2)cc1 ; *c1ccc(-c2ccc(*)cc2)cc1 ; *c1ccc(-c2ccc(-n3cc(*)nn3)cc2)cc1 ; *c1ccc(-c2ccc(-c3cn(*)nn3)cc2)cc1]

In some embodiments of formula (XI), Ar is optionally substituted monocyclic heteroaryl. In some embodiments of formula (XI), Ar is triazole and wherein the compound is of formula (XVa) or (XVb):

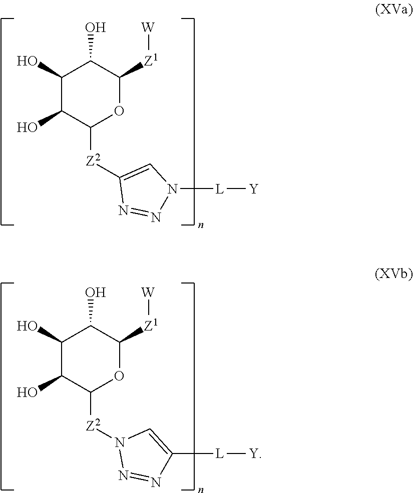

In some embodiments of formula (XVa) or (XVb), Z² is O or S. In some embodiments of formula (XVa) or (XVb), Z² is CH₂.

In some embodiments of formula (XI)-(XVb), n is at least 2, and L is a branched linker that covalently links each Ar group to Y. In some embodiments of formula (XI)-(XVb), n is 2 to 20, such as n is 2 to 10, 2 to 6, e.g., 2 or 3.

In some embodiments of formula (XI)-(XVb), n is 20 to 500 (e.g., 20 to 400, 20 to 300, or 20 to 200, or 50 to 500, or 100 to 500); and L is an α-amino acid polymer (e.g., poly-L-lysine) wherein a multitude of —Ar—Z³-groups are covalently linked to the polymer backbone via sidechain groups (e.g., via conjugation to the sidechain amino groups of lysine residues).

In some embodiments of formula (XI)-(XVb), n is at least 2 and each Z³ linking moiety is separated from every other Z³ linking moiety by a chain of at least 16 consecutive atoms via linker L, such as by a chain of at least 20, at least 25, or at least 30 consecutive atoms, and in some cases by a chain of up to 100 consecutive atoms.

In some embodiments of formula (XI)-(XVb), the compound is of formula (XVI):

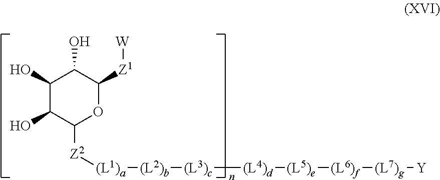

or a salt thereof,
wherein:

n is 1 to 500;

each L¹ to L⁷ is independently a linking moiety that together provide a linear or branched linker between the n Z² groups and Y, and wherein -(L¹)ₐ- comprises the linking moiety Ar that is optionally substituted aryl or heteroaryl group;

a is 1 or 2; and

b, c, d, e, f, and g are each independently 0, 1, or 2.

In some embodiments of formula (XVI), the linear or branched linker separates each Z² and Y by a chain of at least 16 consecutive atoms, such as at least 20 consecutive atoms, at least 30 consecutive atoms, or 16 up to 100 consecutive atoms.

In some embodiments of formula (XVI), n is 1 to 20, such as 1 to 10, 1 to 6 or 1 to 5. In some embodiments of formula (XVI), n is at least 2, e.g., n is 2 or 3. In some embodiments of formula (XVI), when d is >0, L⁴ is a branched linking moiety that is covalently linked to each L¹ linking moiety.

In some embodiments of formula (XVI), the compound is of formula (XVIa)

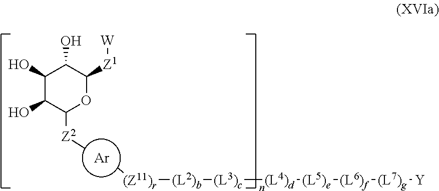

wherein:

Ar is an optionally substituted aryl or heteroaryl group;

Z¹¹ is a linking moiety;

r is 0 or 1; and

n is 1 to 6.

In some embodiments of formula (XVIa), Z¹¹ is a covalent bond, heteroatom, group having a backbone of 1-3 atoms in length (e.g., —NH—, urea, thiourea, ether, amido) or triazole.

In some embodiments of formula (XVIa), Ar is a monocyclic aryl or heteroaryl group. In some embodiments of formula (XVIa), Ar is a bicyclic aryl or heteroaryl group. In some embodiments of formula (XVIa), Ar is a tricyclic aryl or heteroaryl group. In some embodiments of formula (XVIa), Ar is selected from optionally substituted phenyl, optionally substituted biphenyl, optionally substituted naphthalene, optionally substituted triazole, optionally substituted phenyl-triazole, optionally substituted biphenyl-triazole, and optionally substituted naphthalene-triazole. In certain embodiments, Ar is optionally substituted 1,4-phenylene.

In some embodiments of formula (XVIa), Ar substituted with at least one hydroxy.

In some embodiments of formula (XVI)-(XVIa), L¹ or —Ar—(Z¹¹)ᵣ— is selected from:

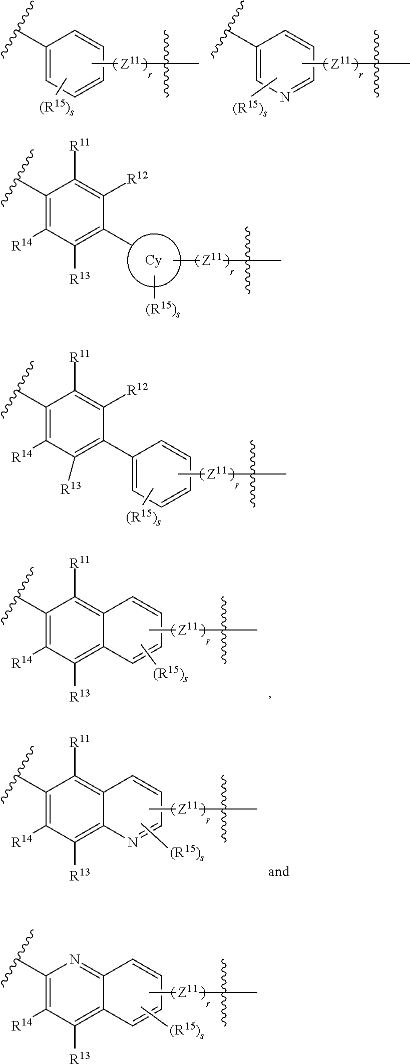

wherein:

Cy is monocyclic aryl or heteroaryl;

r is 0 or 1;

s is 0 to 4 (e.g., 0 to 3, or 0, 1 or 2);

R¹¹ to R¹⁴ and each R¹⁵ are independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —OCOR²⁵, —CONHR²⁵, and —NHCOR²⁵, wherein each R²⁵ is independently selected from H, C₍₁₋₆₎-alkyl and substituted C₍₁₋₆₎-alkyl; and

Z¹¹ is selected from covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X²— and optionally substituted triazole, where X¹ and X² are selected from O, S and NR²³, wherein R²³ and R²⁴ are independently selected from H, C₍₁₋₃₎-alkyl (e.g., methyl) and substituted C₍₁₋₃₎-alkyl.

In some embodiments, r is 0 and Z¹¹ is absent. In some embodiments, r is 1.

In some embodiments of formula (XVI)-(XVIa), L¹ or —Ar—(Z¹¹)ᵣ— is

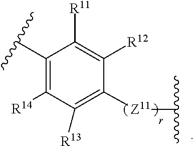

In some embodiments, r is 0 and Z¹¹ is absent. In some embodiments, r is 1.

In some embodiments of formula (XVI)-(XVIa), L¹ or —Ar—(Z¹¹)ᵣ— is

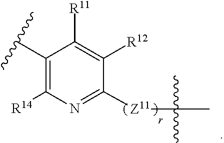

In some embodiments, r is 0 and Z¹¹ is absent. In some embodiments, r is 1.

In some embodiments of formula (XVI)-(XVIa), L¹ or —Ar—(Z¹¹)ᵣ— is

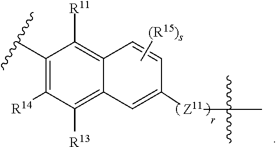

In some embodiments, r is 0 and Z¹¹ is absent. In some embodiments, r is 1.

In some embodiments of formula (XVI)-(XVIa), L¹ or —Ar—(Z¹¹)ᵣ— is

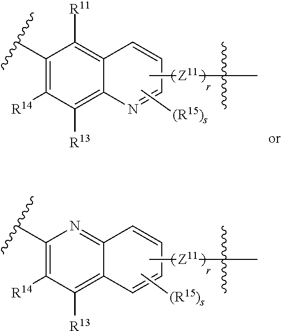

In some embodiments, r is 0 and Z¹¹ is absent. In some embodiments, r is 1.

In some embodiments of formula (XVI)-(XVIa), L¹ or —Ar—(Z¹¹)ᵣ— is selected from:

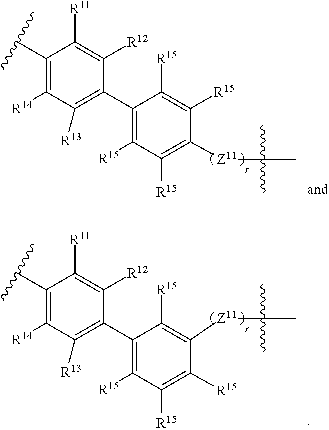

In some embodiments, r is 0 and Z¹¹ is absent. In some embodiments, r is 1 and Z¹¹ is selected from —O—, —NR²³—, —NR²³CO—, CONR²³—, —NR²³CO₂—, —OCONR²³—, —NR²³C(═X¹)NR²³—, —CR²⁴═N—, —CR²⁴═N—X₂—, —NR²³SO₂—, and —SO₂NR²³—; wherein X¹ and X² are selected from O, S and NR²³, and each R²³ and R²⁴ is independently selected from H, C₍₁₋₃₎-alkyl (e.g., methyl) and substituted C₍₁₋₃₎-alkyl.

In some embodiments, r is 1 and Z¹¹ is

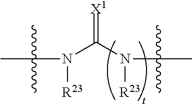

wherein:

X¹ is O or S;

t is 0 or 1; and

each R²³ is independently selected from H, C₍₁₋₃₎-alkyl (e.g., methyl) and substituted C₍₁₋₃₎-alkyl. In some embodiments, Z¹¹ is —NHC(═X¹)NH—, wherein X¹ is O or S. In some embodiments, r is 1 and Z¹¹ is triazole.

In some embodiments of formula (XI)-(XVIa), Z³ is —N(R²³)SO₂— or —SO₂N(R²³)—.

In some embodiments of formula (XI)-(XVIa), Z³ is —N(R²³)CO— or —CON(R²³)—.

In some embodiments of formula (XI)-(XVIa), the hydrophilic head group W is charged, e.g., capable of forming a salt under aqueuos or physiological conditions. In some embodiments of formula (XI)-(XVIa), the hydrophilic head group W is neutral.

In any one of the embodiments of formula (XI)-(XVIa) described herein, the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

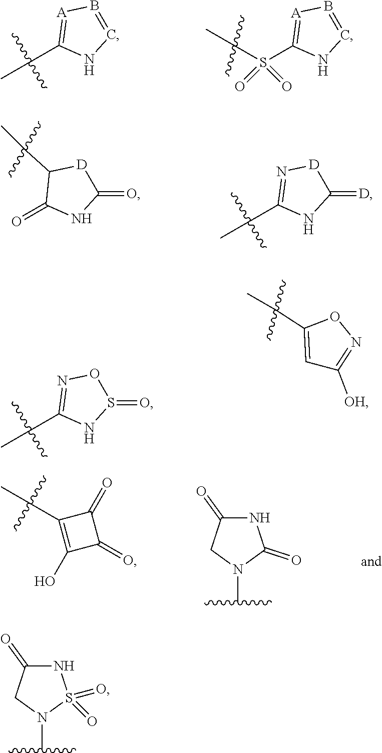

or a salt thereof,
wherein:

R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);

A, B, and C are each independently CH or N; and

D is each independently O or S.

In some embodiments of formula (XI)-(XVIa), the hydrophilic head group W is phosphate or thiophosphate (e.g., —OP═O(OH)₂, —SP═O(OH)₂, —OP═O(SH)(OH), —SP═O(SH)(OH), or —OP═S(OH)₂). In some embodiments of formula (XI)-(XVIa), the hydrophilic head group W is phosphonate or thiophosphonate (e.g., —P═O(OH)₂, —P═S(OH)₂, —P═O(SH)(OH), or —P═S(SH)(OH), or a salt thereof). In some embodiments of formula (XI)-(XVIa), the hydrophilic head group W is sulfonate (e.g., —SO₃H or a salt thereof). In some embodiments of formula (XI)-(XVIa), the hydrophilic head group W is —CO₂H or a salt thereof. In some embodiments of formula (XI)-(XVIa), the hydrophilic head group W is malonate (e.g., —CH(COOH)₂ or a salt thereof).

In some embodiments of formula (XI)-(XVIa), the hydrophilic head group W comprises a 5-membered heterocycle, such as

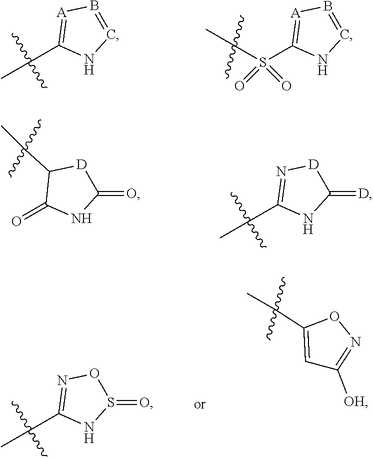

or a salt thereof.

Exemplary hydrophilic head group W are shown in the X groups of Table 1, and the compounds of Tables 5-7B.

In some embodiments of formula (XI)-(XVIa), the linking moiety (Z¹) that connects the hydrophilic head group W to the mannose ring is —(CH₂)ᵣ— where j is 1-3. In some embodiments, j is 2. In some embodiments of formula (XI)-(XVIa), the linking moiety (Z¹) that connects the hydrophilic head group W to the mannose ring is —CH═CH—.

In some embodiments of formula (XI)-(XVIa), the linking moiety (Z²) that connects the mannose ring to the Ar group is O or S. In some embodiments of formula (XI)-(XVIa), Z² is —NR²¹—, where R²¹ is selected from H, and optionally substituted (C₁-C₆)alkyl. In some embodiments of formula (XI)-(XVIa), Z² is —NH—. In some embodiments of formula (XI)-(XVIa), Z² is —C(R²²)₂—, where each R²² is independently selected from H, halogen (e.g., F) and optionally substituted (C₁-C₆)alkyl. In some embodiments of formula (XI)-(XVIa), Z² is CH₂. In some embodiments of formula (XI)-(XVIa), Z² is —CF₂— or —C(CH₃)₂—.

In some embodiments of formula (XI)-(XVIa), Z¹ is selected from —(CH₂)ⱼ— and —CH═CH—; j is 1 to 3; and Z² is selected from O and CH₂.

In some embodiments of formula (XI)-(XVIa), Z¹ is —(CH₂)ⱼ—; j is 2; and Z² is O.

In some embodiments of formula (XI)-(XVIa), Z¹ is —(CH₂)ⱼ—; j is 2; and Z² is CH₂.

In some embodiments of formula (XI)-(XVIa), Z¹ is —CH═CH—; and Z² is O.

In some embodiments of formula (XI)-(XVIa), Z¹ is —CH═CH—; and Z² is CH₂.

As summarized above, the M6PR binding moiety (X) of the compounds of this disclosure (e.g., of formula (Ia)) can include a mannose ring or analog thereof described by the following structure:

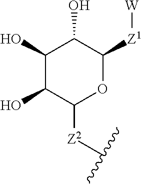

where:

W is a hydrophilic head group;

Z¹ is selected from optionally substituted (C₁-C₃)alkylene and optionally substituted ethenylene;

Z² is selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

The mannose ring or analog thereof of the M6PR binding moiety can be incorporated into the compounds of this disclosure by attachment to the Z² group via a linking moiety. It is understood that in the compounds of formula (Ia), the group or linking moiety attached to Z² can, in some cases, be considered to be part of the M6PR binding moiety (X) and provide for desirable binding to the M6PR. See e.g., formula (XI)-(XVIa), where an aryl or heteroaryl linking moiety is attached to the mannose ring or analog via the Z² group. In certain other cases, the group or linking moiety attached to Z² can be considered part of the linker L of formula (Ia).

In some embodiments of the M6PR binding compounds of this disclosure, e.g., a compound of formula (Ia), the M6PR binding moiety X comprises the group of formula (IIIa), (IIIb), (IIIc), or (IIId):

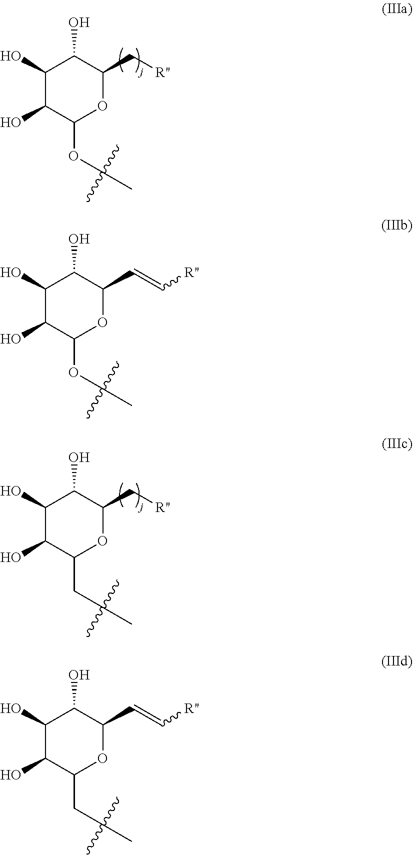

wherein R″ (e.g., a hydrophilic head group) is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

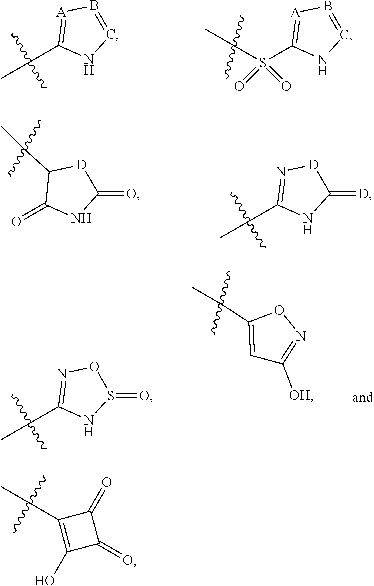

wherein j is an integer of 1 to 3;
wherein R¹ and R² are each independently hydrogen, halo, or CN;
wherein R³ and R⁴ are each independently C₁₋₆ alkyl; and
wherein A, B, and C are each independently CH or N; and D is each independently O or S.

In some embodiments of formula (IIIa), (IIIb), (IIIc), or (IIId), R″ is selected from the group consisting of —OH, —CR¹R²OH, —P═O(OH)₂, P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

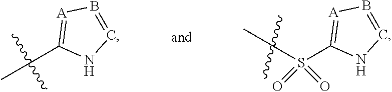

j is an integer of 1 to 3;

R¹ and R² are each independently hydrogen, halo, or CN;

R³ and R⁴ are each independently C₁₋₆ alkyl; and

A, B, and C are each independently CH or N.

In certain embodiments, X comprises the group of formula (IIIa′), (IIIa″), (IIIb′), (IIIb″), (IIIc′), (IIIc″), (IIId′) or (IIId″):

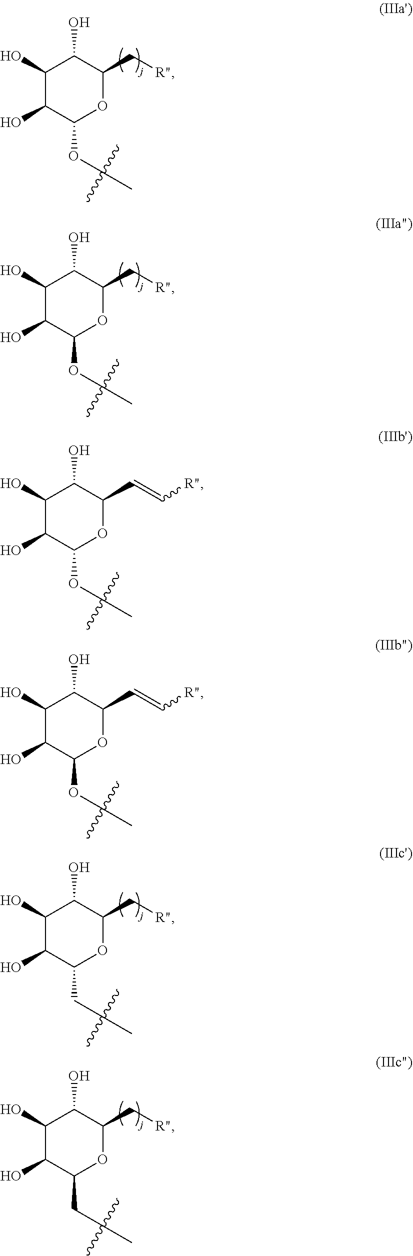

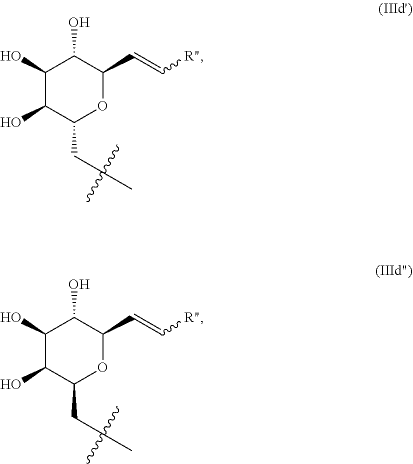

wherein R″ is as defined herein and wherein j is an integer of 1 to 3.

In certain embodiments, X is of formula (IIIa′), (IIIa″), (IIIb′), or (IIIb″). In certain embodiments, X is of formula (IIIc′), (IIIc″), (IIId′) or (IIId″). In certain embodiments, X is of formula (IIIa′) or (IIIa″). In certain embodiments, X is of formula (IIIb′) or (IIIb″). In certain embodiments, X is of formula (IIIc′) or (IIIc″). In certain embodiments, X is of formula (IIId′) or (IIId″). In certain embodiments, X is of formula (IIIa′). In one embodiment, X is of formula (IIIa″). In certain embodiments, X is of formula (IIIb′). In one embodiment, X is of formula (IIIb″). In certain embodiments, X is of formula (IIIc′). In one embodiment, X is of formula (IIIc″). In certain embodiments, X is of formula (IIId′). In one embodiment, X is of formula (IIId″). In certain embodiments, X is of formula (IIIe).

In one embodiment, j is 1 or 2. In another embodiment, j is 2 or 3. In another embodiment, j is 1. In another embodiment, j is 2. In yet another embodiment, j is 3.

In certain embodiments, R″ is selected from the group consisting of —OH, —CR¹R²OH, —P═O(OH)₂, P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

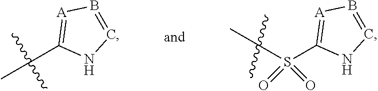

wherein R¹ and R² are each independently hydrogen, halo, or CN;
wherein R³ and R⁴ are each independently C₁₋₆ alkyl; and
wherein A, B, and C are each independently CH or N. In certain embodiments, R″ is not OH.

In certain embodiments, R″ is selected from the group consisting of —OH, —CR¹R²OH, —P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

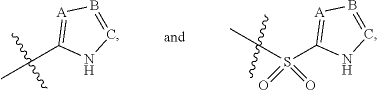

In certain embodiments, R″ is selected from the group consisting of —CR¹R²OH, —P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

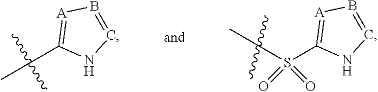

In certain embodiments, R″ is selected from the group consisting of —P═O(OH)₂, P(═O)R¹OH, and —(CR¹R²)—P═O(OH)₂. In certain embodiments, R″ is selected from the group consisting of —SO₂OH, —OSO₂OH, —CONHSO₂R³, —SO₂R³, —SOR³R⁴, —SO₂NH₂, —SO₂NHR³, —SO₂NR³R⁴, and —NHSO₂R³. In certain embodiments, R″ is —OH, or —CR¹R²OH In certain embodiments, R″ is selected from the group consisting of —COOH, —CONH₂, —CONHR¹, —CONR³R⁴, —CH(COOH)₂, —CR¹R²COOH, and —NHCOR³.

In certain embodiments of formula (Ia), X comprises the group of formula (IIIa-1) or (IIIb-1):

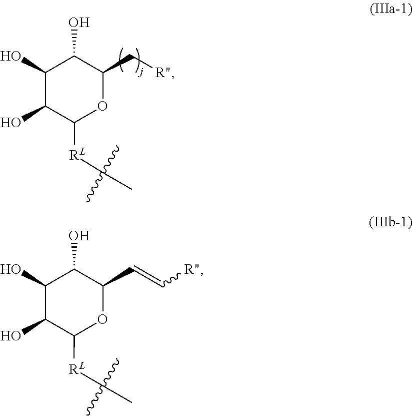

wherein:

R^{L} is —O—, —NH— or —CH₂—;

R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂,

SO₂NHR³, —SO₂NR³R⁴, —SO₂NHCOR³, —NHCOR³, —NHC(O)NHS(O)₂R³, —NHSO₂R³,

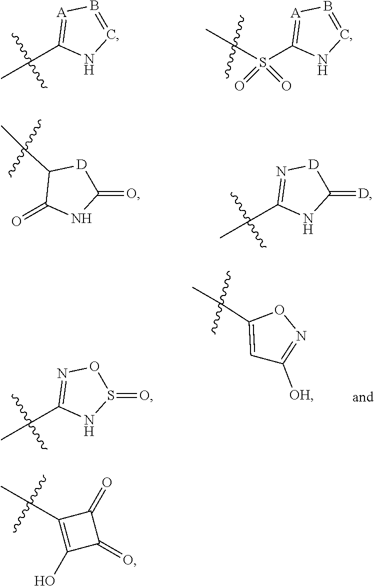

j is an integer of 1 to 3;

R¹ and R² are each independently hydrogen, halo, or CN;

R³ and R⁴ are each independently C₁₋₆ alkyl;

A, B, and C are each independently CH or N; and

D is each independently O or S.

In certain embodiments of formula (Ia), wherein X is of formula (IIIa-1) or (IIIb-1), when R^{L} is —O—, R″ is

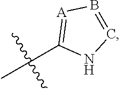

and B and C are N, then j is 2.

In certain embodiments of formula (Ia), wherein X is of formula (IIIa-1) or (IIIb-1), when R^{L} is —O— and R″ is —CR¹R²COOH, R¹ and R² are not both hydrogen.

In certain embodiments of formula (Ia), wherein X is of formula (IIIa-1) or (IIIb-1), when R^{L} is —O—, R″ is

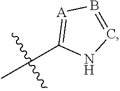

and B and C are N, then j is 2; and when R^{L} is —O— and R″ is —CR¹R²COOH, R¹ and R² are not both hydrogen.

In certain embodiments of the formula (Ia), X is of formula (IIIa-1) or (IIIb-1), R^{L} is —NH— or —CH₂— and R″ and the remaining variables are as described for formula (Ia).

In certain embodiments of the formula (Ia), X is of formula (IIIa-1) or (IIIb-1), and when R¹ is —O—, R″ is

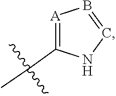

and B and C are N, then j is 2 and provided when R¹ is —O—, R″ is —CR¹R²COOH, R¹ and R² are not both hydrogen.

In certain embodiments, provided herein are compounds of the formula (Ia), wherein X is of formula (IIIa-1) or (IIIb-1), wherein R¹ is —O—, —NH— or —CH₂— and R″ is selected from the group consisting of, —P═O(OH)₂, P(═O)R¹OH, —PH(═O)OH, —(CR¹R²)—P═O(OH)₂, —S(O)OH, —OSO₂OH, —CONH(OH), —CONH(OR³)—CONHSO₂R³, —CONHSO₂NR³R⁴, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

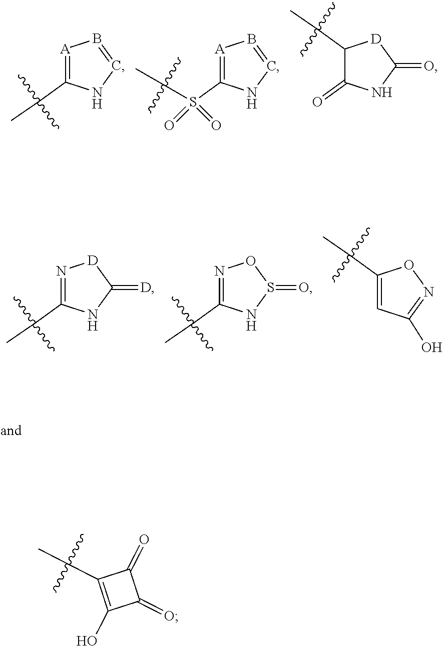

and the remaining variables are as described for formula (Ia).

Exemplary moieties that bind the M6PR (X1 to X27), and synthons which can be utilized in the preparation of compounds of this disclosure that include the M6PR ligand of interest are shown in Table 1.

TABLE 1
Exemplary M6PR binding ligands (X)

| Exemplary X for M6PR binding | | |
|---|---|---|
| compounds | | |
| # | Structure | Exemplary Synthetic precursors |
| X1 | 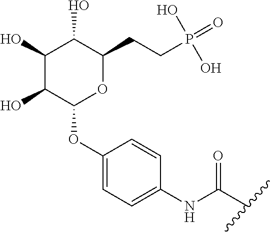 [SMILES: *C(=O)Nc1ccc(O[C@H]2O[C@H](CCP(=O)(O)O)[C@@H](O)[C@H](O)[C@@H]2O)cc1] | 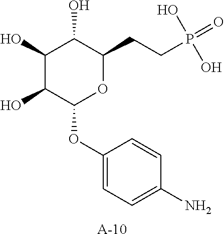 |
| X2 | 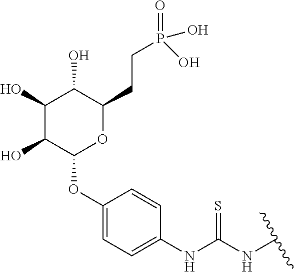 [SMILES: *NC(=S)Nc1ccc(O[C@H]2O[C@H](CCP(=O)(O)O)[C@@H](O)[C@H](O)[C@@H]2O)cc1] | 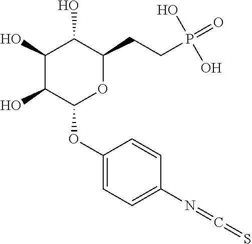 [SMILES: O=P(O)(O)CC[C@H]1O[C@H](Oc2ccc(N=C=S)cc2)[C@@H](O)[C@@H](O)[C@@H]1O] |
| | | 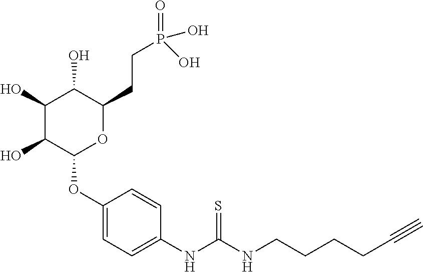 [SMILES: C#CCCCCNC(=S)Nc1ccc(O[C@H]2O[C@H](CCP(=O)(O)O)[C@@H](O)[C@H](O)[C@@H]2O)cc1] |
| X3 | 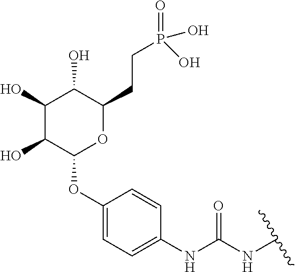 [SMILES: *NC(=O)Nc1ccc(O[C@H]2O[C@H](CCP(=O)(O)O)[C@@H](O)[C@H](O)[C@@H]2O)cc1] | 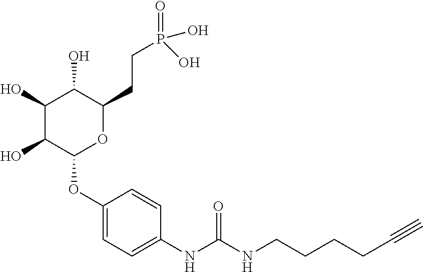 [SMILES: C#CCCCCNC(=O)Nc1ccc(O[C@H]2O[C@H](CCP(=O)(O)O)[C@@H](O)[C@H](O)[C@@H]2O)cc1] |
| X4 | 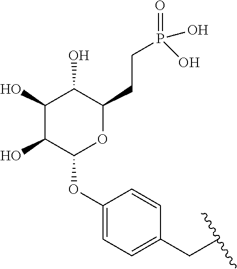 [SMILES: *Cc1ccc(O[C@H]2O[C@H](CCP(=O)(O)O)[C@@H](O)[C@H](O)[C@@H]2O)cc1] | 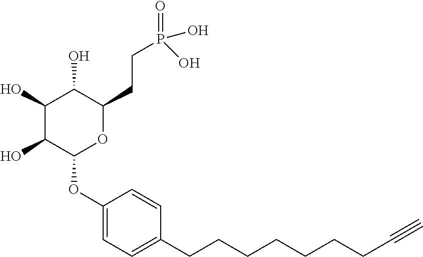 [SMILES: C#CCCCCCCCc1ccc(O[C@H]2O[C@H](CCP(=O)(O)O)[C@@H](O)[C@H](O)[C@@H]2O)cc1] |
| | | 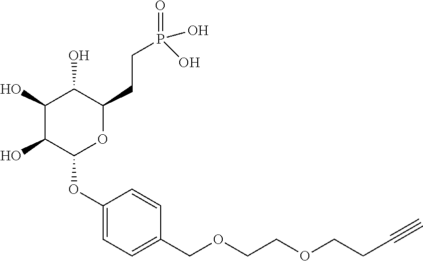 [SMILES: C#CCCOCCOCc1ccc(O[C@H]2O[C@H](CCP(=O)(O)O)[C@@H](O)[C@H](O)[C@@H]2O)cc1] |
| X5 | 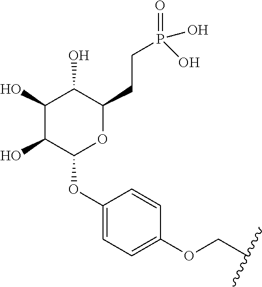 [SMILES: *COc1ccc(O[C@H]2O[C@H](CCP(=O)(O)O)[C@@H](O)[C@H](O)[C@@H]2O)cc1] | 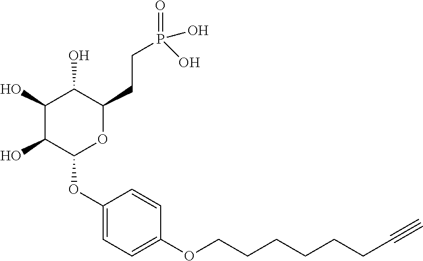 [SMILES: C#CCCCCCCOc1ccc(O[C@H]2O[C@H](CCP(=O)(O)O)[C@@H](O)[C@H](O)[C@@H]2O)cc1] |
| X6 | 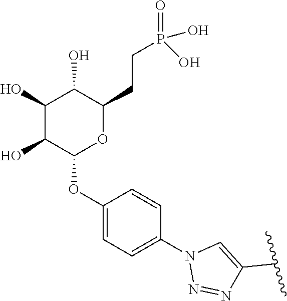 [SMILES: *c1cn(-c2ccc(O[C@H]3O[C@H](CCP(=O)(O)O)[C@@H](O)[C@H](O)[C@@H]3O)cc2)nn1] | 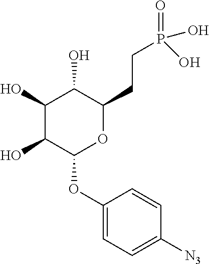 [SMILES: [N-]=[N+]=Nc1ccc(O[C@H]2O[C@H](CCP(=O)(O)O)[C@@H](O)[C@H](O)[C@@H]2O)cc1] |
| X7 | 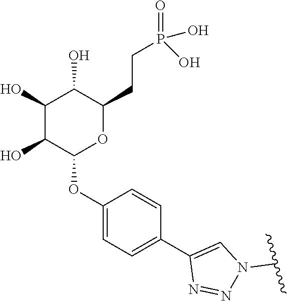 [SMILES: *n1cc(-c2ccc(O[C@H]3O[C@H](CCP(=O)(O)O)[C@@H](O)[C@H](O)[C@@H]3O)cc2)nn1] | 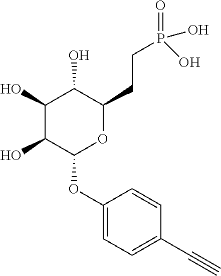 [SMILES: C#Cc1ccc(O[C@H]2O[C@H](CCP(=O)(O)O)[C@@H](O)[C@H](O)[C@@H]2O)cc1] |
| X8 | 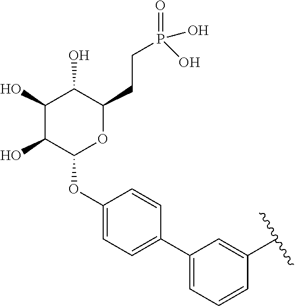 [SMILES: *c1cccc(-c2ccc(O[C@H]3O[C@H](CCP(=O)(O)O)[C@@H](O)[C@H](O)[C@@H]3O)cc2)c1] | 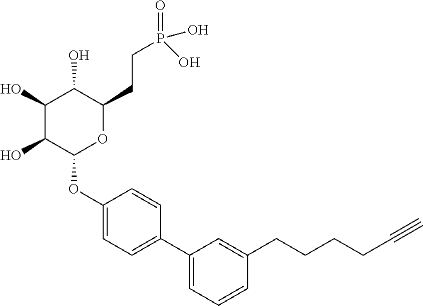 [SMILES: C#CCCCCc1cccc(-c2ccc(O[C@H]3O[C@H](CCP(=O)(O)O)[C@@H](O)[C@H](O)[C@@H]3O)cc2)c1] |
| X9 | 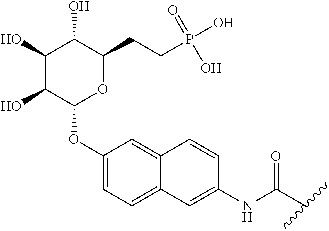 [SMILES: *C(=O)Nc1ccc2cc(O[C@H]3O[C@H](CCP(=O)(O)O)[C@@H](O)[C@H](O)[C@@H]3O)ccc2c1] | 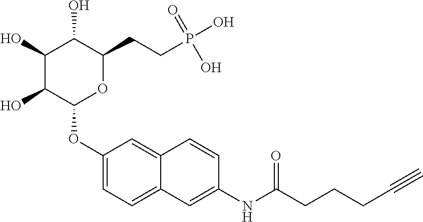 [SMILES: C#CCCCC(=O)Nc1ccc2cc(O[C@H]3O[C@H](CCP(=O)(O)O)[C@@H](O)[C@H](O)[C@@H]3O)ccc2c1] |
| X10 | 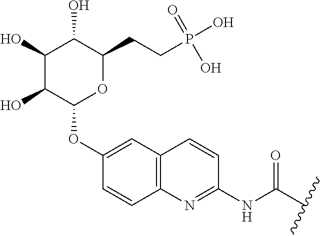 [SMILES: *C(=O)Nc1ccc2cc(O[C@H]3O[C@H](CCP(=O)(O)O)[C@@H](O)[C@H](O)[C@@H]3O)ccc2n1] | 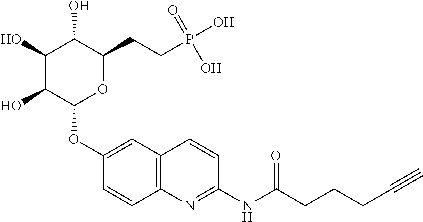 [SMILES: C#CCCCC(=O)Nc1ccc2cc(O[C@H]3O[C@H](CCP(=O)(O)O)[C@@H](O)[C@H](O)[C@@H]3O)ccc2n1] |
| X11 | 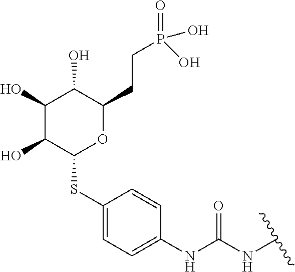 [SMILES: *NC(=O)Nc1ccc(S[C@H]2O[C@H](CCP(=O)(O)O)[C@@H](O)[C@H](O)[C@@H]2O)cc1] | 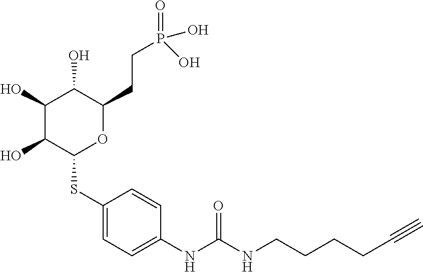 [SMILES: C#CCCCCNC(=O)Nc1ccc(S[C@H]2O[C@H](CCP(=O)(O)O)[C@@H](O)[C@H](O)[C@@H]2O)cc1] |
| X12 | 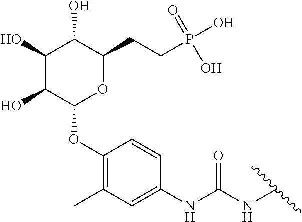 [SMILES: *NC(=O)Nc1ccc(O[C@H]2O[C@H](CCP(=O)(O)O)[C@@H](O)[C@H](O)[C@@H]2O)c(C)c1] | 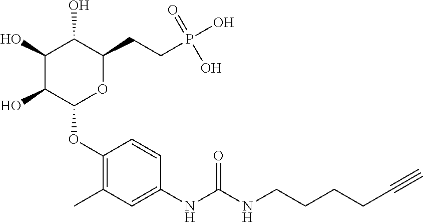 [SMILES: C#CCCCCNC(=O)Nc1ccc(O[C@H]2O[C@H](CCP(=O)(O)O)[C@@H](O)[C@H](O)[C@@H]2O)c(C)c1] |
| X13 | 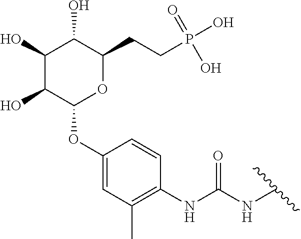 [SMILES: *NC(=O)Nc1ccc(O[C@H]2O[C@H](CCP(=O)(O)O)[C@@H](O)[C@H](O)[C@@H]2O)cc1C] | 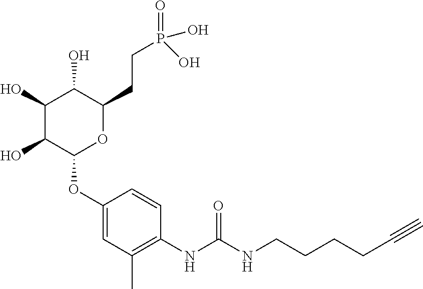 [SMILES: C#CCCCCNC(=O)Nc1ccc(O[C@H]2O[C@H](CCP(=O)(O)O)[C@@H](O)[C@H](O)[C@@H]2O)cc1C] |
| X14 | 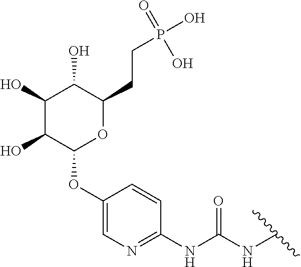 [SMILES: *NC(=O)Nc1ccc(O[C@H]2O[C@H](CCP(=O)(O)O)[C@@H](O)[C@H](O)[C@@H]2O)cn1] | 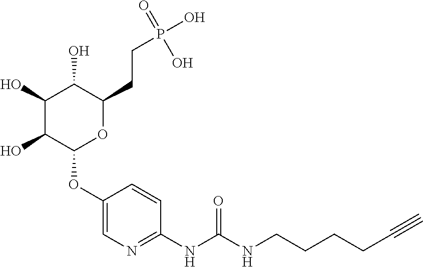 [SMILES: C#CCCCCNC(=O)Nc1ccc(O[C@H]2O[C@H](CCP(=O)(O)O)[C@@H](O)[C@H](O)[C@@H]2O)cn1] |
| X15 | 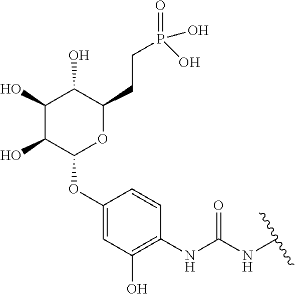 [SMILES: *NC(=O)Nc1ccc(O[C@H]2O[C@H](CCP(=O)(O)O)[C@@H](O)[C@H](O)[C@@H]2O)cc1O] | 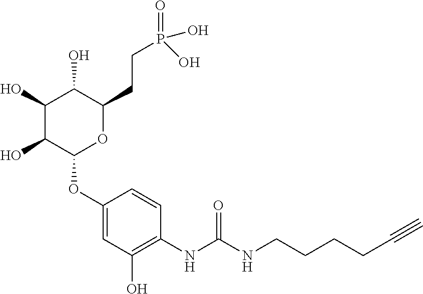 [SMILES: C#CCCCCNC(=O)Nc1ccc(O[C@H]2O[C@H](CCP(=O)(O)O)[C@@H](O)[C@H](O)[C@@H]2O)cc1O] |
| X16 | 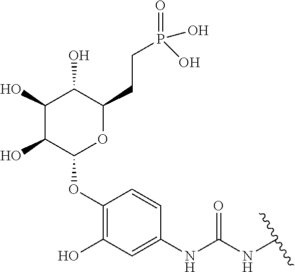 [SMILES: *NC(=O)Nc1ccc(O[C@H]2O[C@H](CCP(=O)(O)O)[C@@H](O)[C@H](O)[C@@H]2O)c(O)c1] | 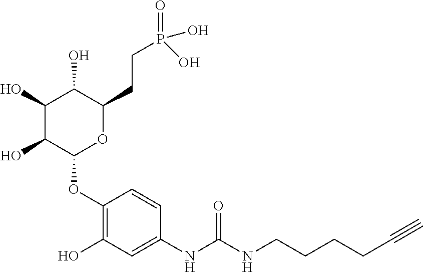 [SMILES: C#CCCCCNC(=O)Nc1ccc(O[C@H]2O[C@H](CCP(=O)(O)O)[C@@H](O)[C@H](O)[C@@H]2O)c(O)c1] |
| X17 | 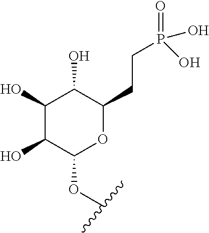 [SMILES: *O[C@H]1O[C@H](CCP(=O)(O)O)[C@@H](O)[C@H](O)[C@@H]1O] | 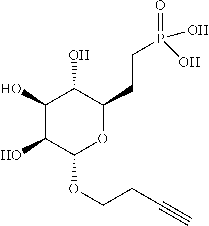 [SMILES: C#CCCO[C@H]1O[C@H](CCP(=O)(O)O)[C@@H](O)[C@H](O)[C@@H]1O] |
| X18 | 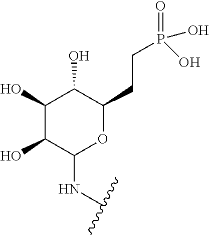 [SMILES: *NC1O[C@H](CCP(=O)(O)O)[C@@H](O)[C@H](O)[C@@H]1O] | 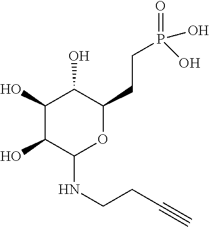 [SMILES: C#CCCNC1O[C@H](CCP(=O)(O)O)[C@@H](O)[C@H](O)[C@@H]1O] |
| X19 | 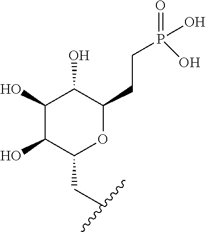 [SMILES: *C[C@H]1O[C@H](CCP(=O)(O)O)[C@@H](O)[C@H](O)[C@@H]1O] | 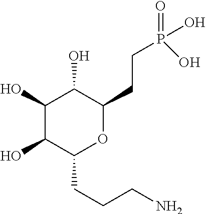 [SMILES: NCCC[C@H]1O[C@H](CCP(=O)(O)O)[C@@H](O)[C@H](O)[C@@H]1O] |
| X20 | 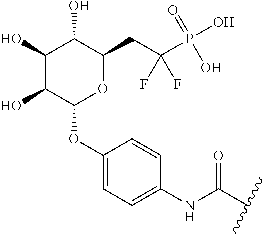 [SMILES: *C(=O)Nc1ccc(O[C@H]2O[C@H](CC(F)(F)P(=O)(O)O)[C@@H](O)[C@H](O)[C@@H]2O)cc1] | 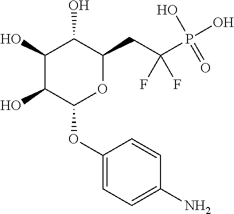 [SMILES: Nc1ccc(O[C@H]2O[C@H](CC(F)(F)P(=O)(O)O)[C@@H](O)[C@H](O)[C@@H]2O)cc1] |
| X21 | 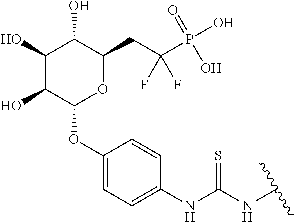 [SMILES: *NC(=S)Nc1ccc(O[C@H]2O[C@H](CC(F)(F)P(=O)(O)O)[C@@H](O)[C@H](O)[C@@H]2O)cc1] | 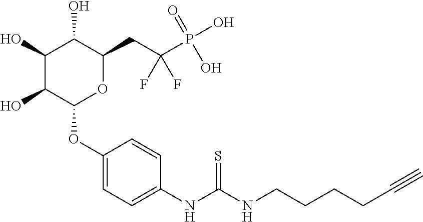 [SMILES: C#CCCCCNC(=S)Nc1ccc(O[C@H]2O[C@H](CC(F)(F)P(=O)(O)O)[C@@H](O)[C@H](O)[C@@H]2O)cc1] |
| X22 | 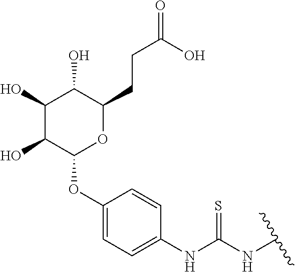 [SMILES: *NC(=S)Nc1ccc(O[C@H]2O[C@H](CCC(=O)O)[C@@H](O)[C@H](O)[C@@H]2O)cc1] | 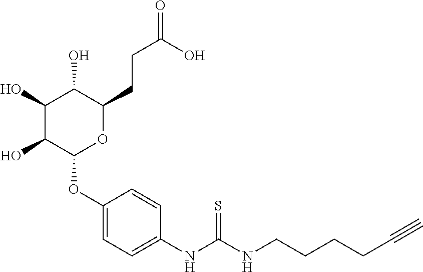 [SMILES: C#CCCCCNC(=S)Nc1ccc(O[C@H]2O[C@H](CCC(=O)O)[C@@H](O)[C@H](O)[C@@H]2O)cc1] |
| X23 | 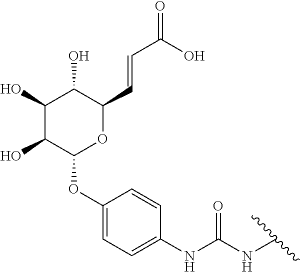 [SMILES: *NC(=O)Nc1ccc(O[C@H]2O[C@H](/C=C/C(=O)O)[C@@H](O)[C@H](O)[C@@H]2O)cc1] | 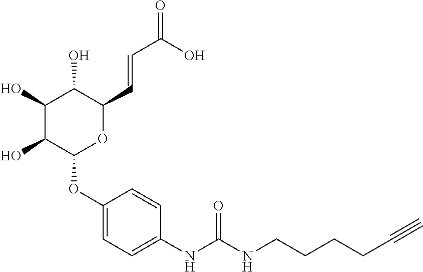 [SMILES: C#CCCCCNC(=O)Nc1ccc(O[C@H]2O[C@H](/C=C/C(=O)O)[C@@H](O)[C@H](O)[C@@H]2O)cc1] |
| X24 | 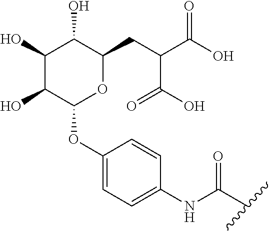 [SMILES: *C(=O)Nc1ccc(O[C@H]2O[C@H](CC(C(=O)O)C(=O)O)[C@@H](O)[C@H](O)[C@@H]2O)cc1] | 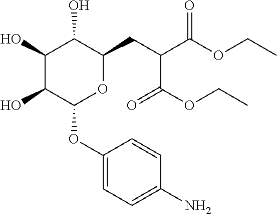 [SMILES: CCOC(=O)C(C[C@H]1O[C@H](Oc2ccc(N)cc2)[C@@H](O)[C@@H](O)[C@@H]1O)C(=O)OCC] |
| X25 | 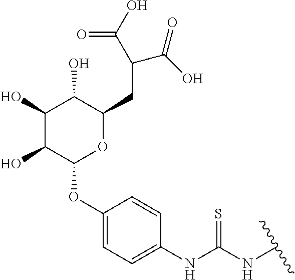 [SMILES: *NC(=S)Nc1ccc(O[C@H]2O[C@H](CC(C(=O)O)C(=O)O)[C@@H](O)[C@H](O)[C@@H]2O)cc1] | 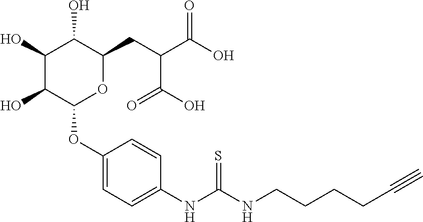 [SMILES: C#CCCCCNC(=S)Nc1ccc(O[C@H]2O[C@H](CC(C(=O)O)C(=O)O)[C@@H](O)[C@H](O)[C@@H]2O)cc1] |
| X26 | 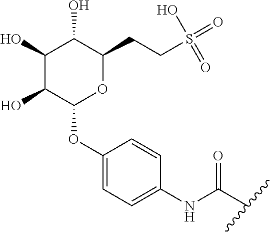 [SMILES: *C(=O)Nc1ccc(O[C@H]2O[C@H](CCS(=O)(=O)O)[C@@H](O)[C@H](O)[C@@H]2O)cc1] | 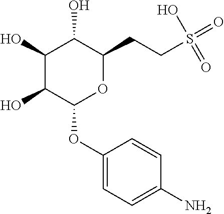 [SMILES: Nc1ccc(O[C@H]2O[C@H](CCS(=O)(=O)O)[C@@H](O)[C@H](O)[C@@H]2O)cc1] |
| X27 | 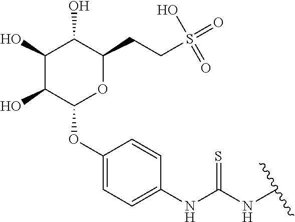 [SMILES: *NC(=S)Nc1ccc(O[C@H]2O[C@H](CCS(=O)(=O)O)[C@@H](O)[C@H](O)[C@@H]2O)cc1] | 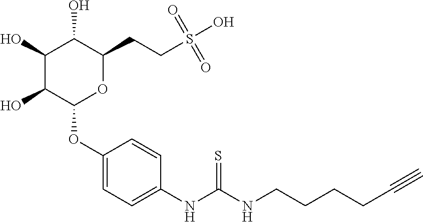 [SMILES: C#CCCCCNC(=S)Nc1ccc(O[C@H]2O[C@H](CCS(=O)(=O)O)[C@@H](O)[C@H](O)[C@@H]2O)cc1] |
| X28 | 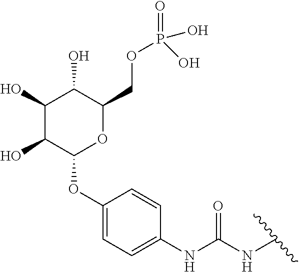 [SMILES: *NC(=O)Nc1ccc(O[C@H]2O[C@H](COP(=O)(O)O)[C@@H](O)[C@H](O)[C@@H]2O)cc1] | 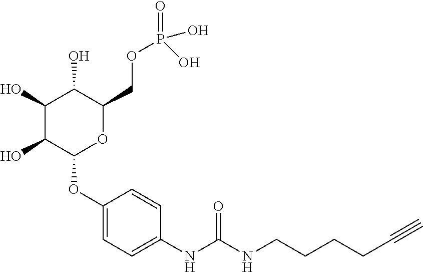 [SMILES: C#CCCCCNC(=O)Nc1ccc(O[C@H]2O[C@H](COP(=O)(O)O)[C@@H](O)[C@H](O)[C@@H]2O)cc1] |
| X29 | 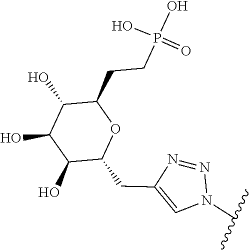 [SMILES: *n1cc(C[C@H]2O[C@H](CCP(=O)(O)O)[C@@H](O)[C@H](O)[C@@H]2O)nn1] | 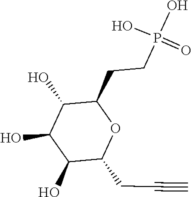 [SMILES: C#CC[C@H]1O[C@H](CCP(=O)(O)O)[C@@H](O)[C@H](O)[C@@H]1O] |
| X30 | 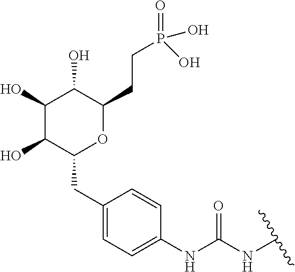 [SMILES: *NC(=O)Nc1ccc(C[C@H]2O[C@H](CCP(=O)(O)O)[C@@H](O)[C@H](O)[C@@H]2O)cc1] | 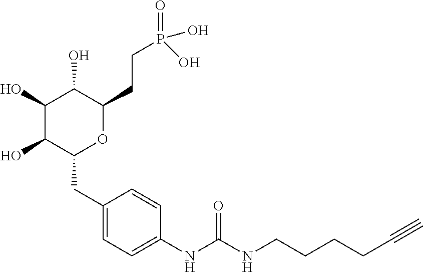 [SMILES: C#CCCCCNC(=O)Nc1ccc(C[C@H]2O[C@H](CCP(=O)(O)O)[C@@H](O)[C@H](O)[C@@H]2O)cc1] |
| X31 | 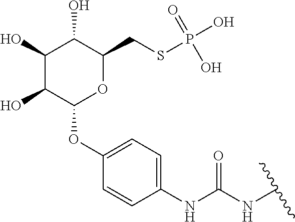 [SMILES: *NC(=O)Nc1ccc(O[C@H]2O[C@H](CSP(=O)(O)O)[C@@H](O)[C@H](O)[C@@H]2O)cc1] | 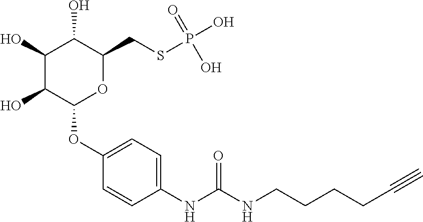 [SMILES: C#CCCCCNC(=O)Nc1ccc(O[C@H]2O[C@H](CSP(=O)(O)O)[C@@H](O)[C@H](O)[C@@H]2O)cc1] |
| X32 | 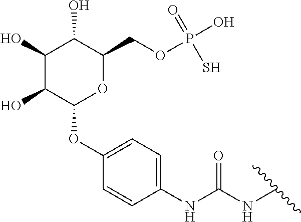 [SMILES: *NC(=O)Nc1ccc(O[C@H]2O[C@H](COP(=O)(O)S)[C@@H](O)[C@H](O)[C@@H]2O)cc1] | 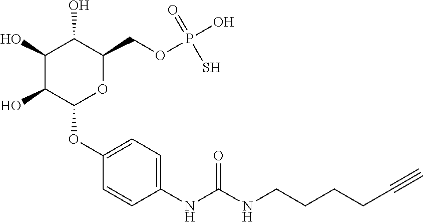 [SMILES: C#CCCCCNC(=O)Nc1ccc(O[C@H]2O[C@H](COP(=O)(O)S)[C@@H](O)[C@H](O)[C@@H]2O)cc1] |
| X32 | 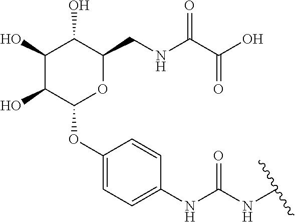 [SMILES: *NC(=O)Nc1ccc(O[C@H]2O[C@H](CNC(=O)C(=O)O)[C@@H](O)[C@H](O)[C@@H]2O)cc1] | 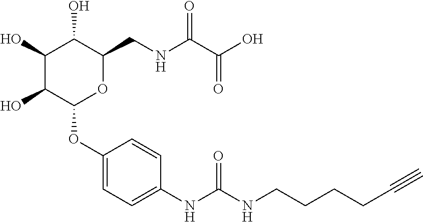 [SMILES: C#CCCCCNC(=O)Nc1ccc(O[C@H]2O[C@H](CNC(=O)C(=O)O)[C@@H](O)[C@H](O)[C@@H]2O)cc1] |
| X33 | 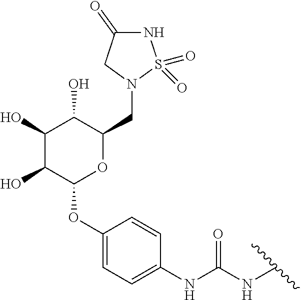 [SMILES: *NC(=O)Nc1ccc(O[C@H]2O[C@H](CN3CC(=O)NS3(=O)=O)[C@@H](O)[C@H](O)[C@@H]2O)cc1] | 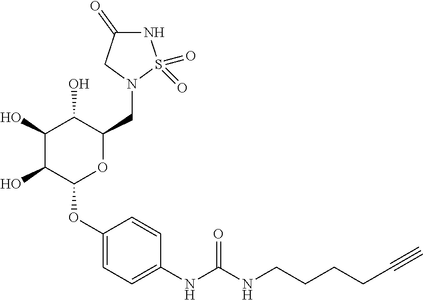 [SMILES: C#CCCCCNC(=O)Nc1ccc(O[C@H]2O[C@H](CN3CC(=O)NS3(=O)=O)[C@@H](O)[C@H](O)[C@@H]2O)cc1] |
| X34 | 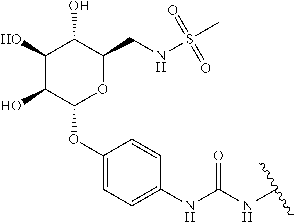 [SMILES: *NC(=O)Nc1ccc(O[C@H]2O[C@H](CNS(C)(=O)=O)[C@@H](O)[C@H](O)[C@@H]2O)cc1] | 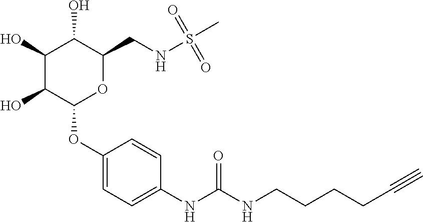 [SMILES: C#CCCCCNC(=O)Nc1ccc(O[C@H]2O[C@H](CNS(C)(=O)=O)[C@@H](O)[C@H](O)[C@@H]2O)cc1] |
| X35 | 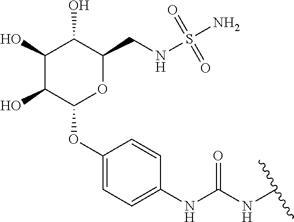 [SMILES: *NC(=O)Nc1ccc(O[C@H]2O[C@H](CNS(N)(=O)=O)[C@@H](O)[C@H](O)[C@@H]2O)cc1] | 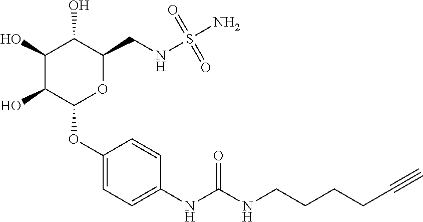 [SMILES: C#CCCCCNC(=O)Nc1ccc(O[C@H]2O[C@H](CNS(N)(=O)=O)[C@@H](O)[C@H](O)[C@@H]2O)cc1] |
| X36 | 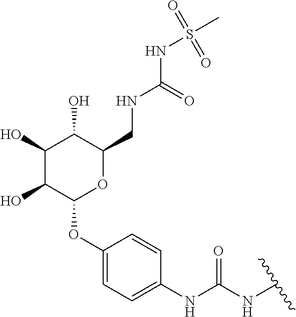 [SMILES: *NC(=O)Nc1ccc(O[C@H]2O[C@H](CNC(=O)NS(C)(=O)=O)[C@@H](O)[C@H](O)[C@@H]2O)cc1] | 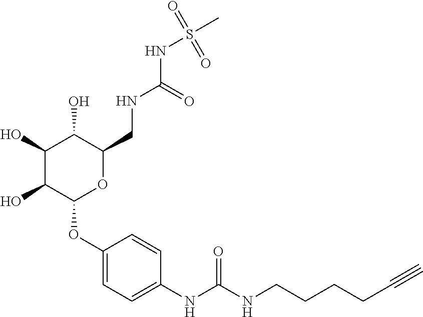 [SMILES: C#CCCCCNC(=O)Nc1ccc(O[C@H]2O[C@H](CNC(=O)NS(C)(=O)=O)[C@@H](O)[C@H](O)[C@@H]2O)cc1] |
| X37 | 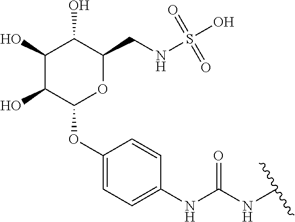 [SMILES: *NC(=O)Nc1ccc(O[C@H]2O[C@H](CNS(=O)(=O)O)[C@@H](O)[C@H](O)[C@@H]2O)cc1] | 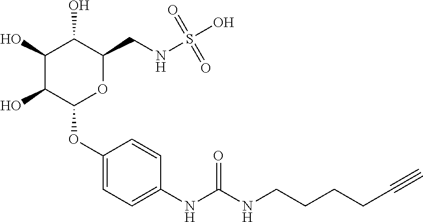 [SMILES: C#CCCCCNC(=O)Nc1ccc(O[C@H]2O[C@H](CNS(=O)(=O)O)[C@@H](O)[C@H](O)[C@@H]2O)cc1] |
| X38 | 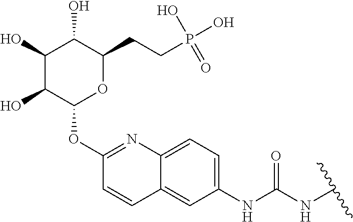 [SMILES: *NC(=O)Nc1ccc2nc(O[C@H]3O[C@H](CCP(=O)(O)O)[C@@H](O)[C@H](O)[C@@H]3O)ccc2c1] | 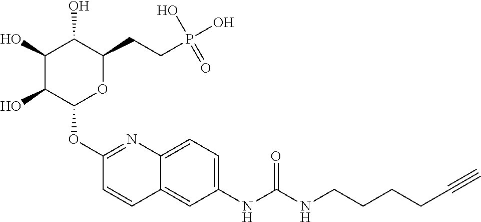 [SMILES: C#CCCCCNC(=O)Nc1ccc2nc(O[C@H]3O[C@H](CCP(=O)(O)O)[C@@H](O)[C@H](O)[C@@H]3O)ccc2c1] |
| X39 | 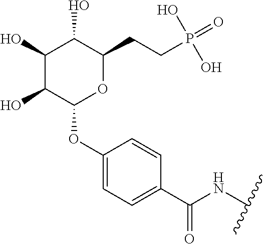 [SMILES: *NC(=O)c1ccc(O[C@H]2O[C@H](CCP(=O)(O)O)[C@@H](O)[C@H](O)[C@@H]2O)cc1] | 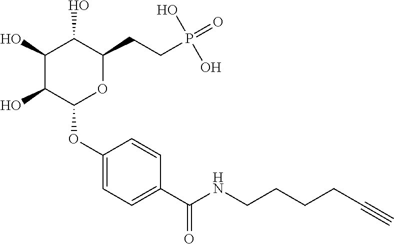 [SMILES: C#CCCCCNC(=O)c1ccc(O[C@H]2O[C@H](CCP(=O)(O)O)[C@@H](O)[C@H](O)[C@@H]2O)cc1] |
| X40 | 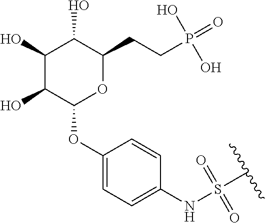 [SMILES: *S(=O)(=O)Nc1ccc(O[C@H]2O[C@H](CCP(=O)(O)O)[C@@H](O)[C@H](O)[C@@H]2O)cc1] | 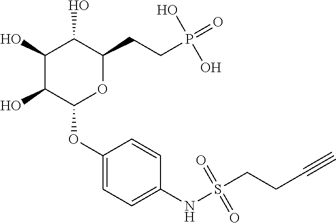 [SMILES: C#CCCS(=O)(=O)Nc1ccc(O[C@H]2O[C@H](CCP(=O)(O)O)[C@@H](O)[C@H](O)[C@@H]2O)cc1] |
| X41 | 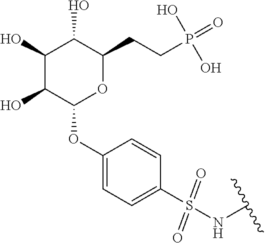 [SMILES: *NS(=O)(=O)c1ccc(O[C@H]2O[C@H](CCP(=O)(O)O)[C@@H](O)[C@H](O)[C@@H]2O)cc1] | 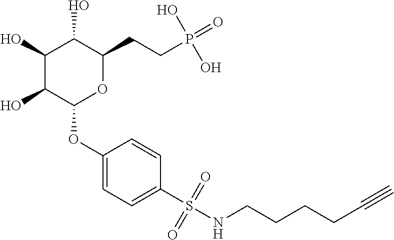 [SMILES: C#CCCCCNS(=O)(=O)c1ccc(O[C@H]2O[C@H](CCP(=O)(O)O)[C@@H](O)[C@H](O)[C@@H]2O)cc1] |
| X42 | 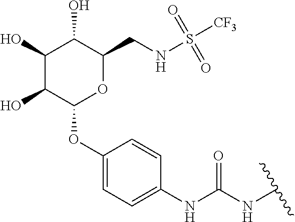 [SMILES: *NC(=O)Nc1ccc(O[C@H]2O[C@H](CNS(=O)(=O)C(F)(F)F)[C@@H](O)[C@H](O)[C@@H]2O)cc1] | 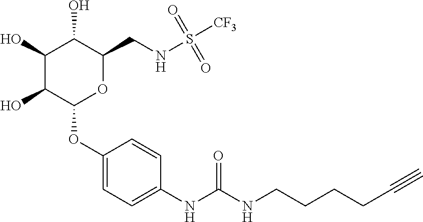 [SMILES: C#CCCCCNC(=O)Nc1ccc(O[C@H]2O[C@H](CNS(=O)(=O)C(F)(F)F)[C@@H](O)[C@H](O)[C@@H]2O)cc1] |

### ASGPR Binding Compounds

As summarized above, this disclosure provides a class of compounds including a ligand moiety that specifically binds to a cell surface asialoglycoprotein receptor” (ASGPR).

The term “asialoglycoprotein receptor” (ASGPR), also known as the Ashwell Morell receptor, means the transmembrane glycoprotein receptor found primarily in hepatocytes which plays an important role in serum glycoprotein homeostasis by mediating the endocytosis and lysosomal degradation of glycoproteins with exposed terminal galactose or N-acetylgalactosamine (GaINAc) residues. ASGPR cycles between endosomes and the cell surface. In particular embodiments, the ASGPR is Homo sapiens asialoglycoprotein receptor 1 (ASGR1) (see, e.g., NCBI Reference Sequence: NM_001197216).

Accordingly, provided herein are ASGPR binding compounds of formula (Ib):

Xₙ-L-Y (Ib)

or a salt thereof,
wherein:

X is a moiety that binds to a cell surface ASGPR (e.g., ASGPR ligand or binding moiety, e.g., as described herein);

n is 1 to 500;

L is a linker of defined length; and

Y is a moiety of interest.

The ASGPR binding moiety (X) of the compounds and conjugates of this disclosure can be a N-acetylgalactosamine (GaINAc), or an analog or derivative of GaINAc. A variety of ligands capable of binding ASGPR can be adapted for use in the compounds and conjugates of this disclosure.

In certain embodiments, each X is independently selected from the group consisting of formula (IIIj), formula (IIIk), formula (IIIl), and formula (IIIm):

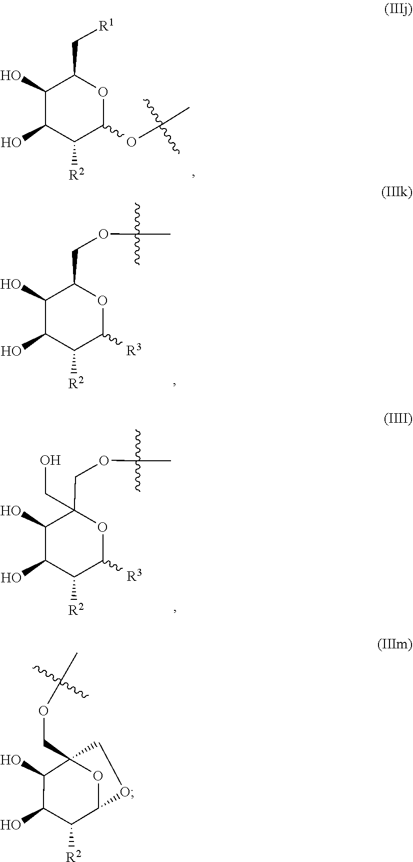

wherein R¹ is —OH, —OC(O)R, or

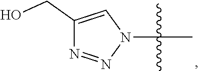

wherein R is C₁₋₆ alkyl;
wherein R² is selected from the group consisting of —NHCOCH₃, —NHCOCF₃, —NHCOCH₂CF₃, —OH, and

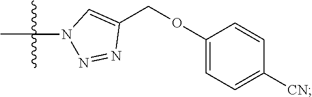 [SMILES: *n1cc(COc2ccc(C#N)cc2)nn1]

and
wherein R³ is selected from the group consisting of —H, —OH, —CH₃, —OCH₃, and OCH₂CH═CH₂.

In certain embodiments, X is of formula (IIIo)

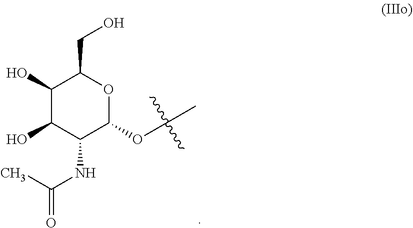 [SMILES: *O[C@H]1O[C@H](CO)[C@H](O)[C@H](O)[C@H]1NC(C)=O]

In certain embodiments, X is of formula:

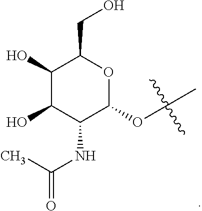 [SMILES: *O[C@H]1O[C@H](CO)[C@H](O)[C@H](O)[C@H]1NC(C)=O]

In certain embodiments, X is of formula (IIIp)

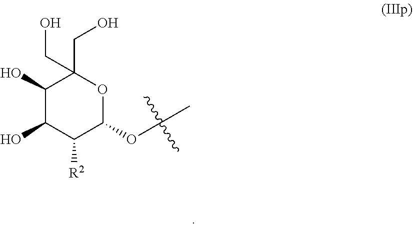

In certain embodiments, X is of formula (IIIo)

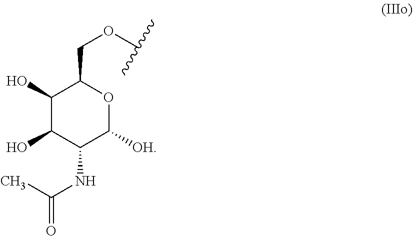 [SMILES: *OC[C@H]1O[C@H](O)[C@H](NC(C)=O)[C@@H](O)[C@H]1O]

In certain embodiments, X is of formula:

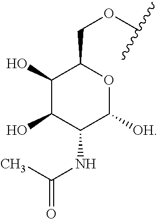 [SMILES: *OC[C@H]1O[C@H](O)[C@H](NC(C)=O)[C@@H](O)[C@H]1O]

In certain embodiments, X is selected from the group consisting of formula (IIIj′), formula (IIIk′), formula (IIIl′), and formula (IIIm′):

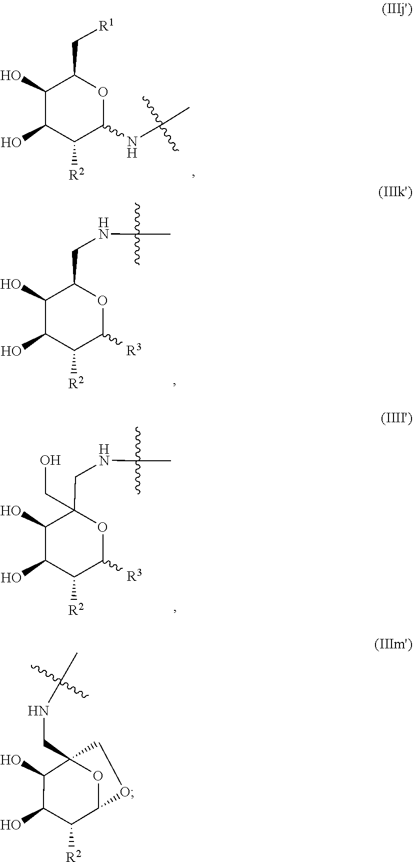

wherein R¹ is —OH, —OC(O)R, or

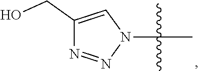

wherein R is C₁₋₆ alkyl;
wherein R² is selected from the group consisting of —NHCOCH₃, —NHCOCF₃, —NHCOCH₂CF₃, —OH, and

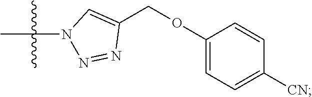 [SMILES: *n1cc(COc2ccc(C#N)cc2)nn1]

and
wherein R³ is selected from the group consisting of —H, —OH, —CH₃, —OCH₃, and —OCH₂CH═CH₂.

In certain embodiments, X is of formula (IIIo′)

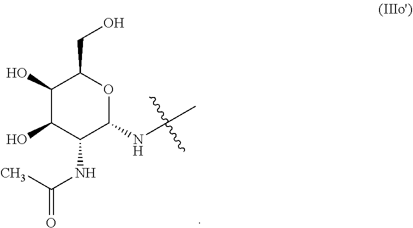 [SMILES: *N[C@H]1O[C@H](CO)[C@H](O)[C@H](O)[C@H]1NC(C)=O]

In certain embodiments, X is of formula (IIIp′)

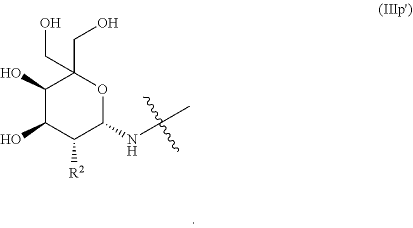

In certain embodiments of the compounds described herein, each X is independently selected from the group consisting of formulas (IIIa), (IIIb), (IIIc), (IIId), (IIIe), (IIIj), (IIIk), (IIIl), (IIIm), (IIIp), (IIIj′), (IIIk′), (IIIl′), (IIIm′), and (IIIp′).

In one embodiment, the compound of formula (Ib) is selected from the compounds of Table 8. In one embodiment, the compound of formula (Ib) is selected from the compounds of Table 9.

Exemplary ASGPR binding compounds of formula (Ib) are shown in Tables 8-9.

### Linkers

The terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieites has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo(ethylene glycol), ether, thioether, disulfide, amide, carbonate, carbamate, tertiary amine, alkyl which may be straight or branched, e.g., methyl, ethyl, n-propyl, 1-methylethyl (iso-propyl), nbutyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest (Y), e.g., a biomolecule (e.g., an antibody) and the other for conjugation to a moiety (noted as X) that binds to a cell surface receptor. For example, if the cell surface receptor is a mannose-6-phosphate receptor (M6PR), then the moiety may be mannose-6-phosphate or a analog of a mannose-6-phosphate moiety. When Y is a polypeptide, the polypeptide conjugation reactive terminus of the linker is in some cases a site that is capable of conjugation to the polypeptide through a cysteine thiol or lysine amine group on the polypeptide, and so is can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker L comprises one or more straight or branched-chain carbon moieties and/or polyether (e.g., ethylene glycol) moieties (e.g., repeating units of —CH₂CH₂O—), and combinations thereof. In certain embodiments, these linkers optionally have amide linkages, urea or thiourea linkages, carbamate linkages, ester linkages, amino linkages, ether linkages, thioether linkages, sulfhydryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In certain embodiments, L is between about 10 Å and about 20 Å in length. In certain embodiments, L is between about 15 Å and about 20 Å in length. In certain embodiments, L is about 15 Å in length. In certain embodiments, L is about 16 Å in length. In certain embodiments, L is about 17 Å in length.

In certain embodiments, L is a linker between about 5 Å and about 500 Å. In certain embodiments, L is between about 10 Å and about 400 Å. In certain embodiments, L is between about 10 Å and about 300 Å. In certain embodiments, L is between about 10 Å and about 200 Å. In certain embodiments, L is between about 10 Å and about 100 Å. In certain embodiments, L is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å. In certain embodiments, L is a linker between about 5 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 500 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 400 Å, which comprises an optionally substituted arylene linked to X, optionally substituted heteroarylene linked to X, optionally substituted heterocyclene linked to X, or optionally substituted cycloalkylene linked to X. In certain embodiments, L is a linker between about 10 Å and about 200 Å, which comprises an optionally substituted arylene linked to X, optionally substituted heteroarylene linked to X, optionally substituted heterocyclene linked to X, or optionally substituted cycloalkylene linked to X.

In certain embodiments, linker L separates X and Y (or Z) by a chain of 4 to 500 consecutive atoms. In certain embodiments, linker L separates X and Y (or Z) by a chain of 4 to 50 consecutive atoms. In certain embodiments, linker L separates X and Y (or Z) by a chain of 6 to 50 consecutive atoms, by a chain of 11 to 50 consecutive atoms, by a chain of 16 to 50 consecutive atoms, by a chain of 21 to 50 consecutive atoms, by a chain of 26 to 50 consecutive atoms, by a chain of 31 to 50 consecutive atoms, by a chain of 36 to 50 consecutive atoms, by a chain of 41 to 50 consecutive atoms, or by a chain of 46 to 50 consecutive atoms. In certain embodiments, linker L separates X and Y (or Z) by a chain of 6 to 50 consecutive atoms. In certain embodiments, linker L separates X and Y (or Z) by a chain of 11 to 50 consecutive atoms. In certain embodiments, linker L separates X and Y (or Z) by a chain of 16 to 50 consecutive atoms. In certain embodiments, linker L separates X and Y (or Z) by a chain of 21 to 50 consecutive atoms. In certain embodiments, linker L separates X and Y (or Z) by a chain of 26 to 50 consecutive atoms. In certain embodiments, linker L separates X and Y (or Z) by a chain of 31 to 50 consecutive atoms. In certain embodiments, linker L separates X and Y (or Z) by a chain of 36 to 50 consecutive atoms. In certain embodiments, linker L separates X and Y (or Z) by a chain of 41 to 50 consecutive atoms. In certain embodiments, linker L separates X and Y (or Z) by a chain of 46 to 50 consecutive atoms.

In certain embodiments, linker L separates X and Y (or Z) by a chain of 4 or 5 consecutive atoms, by a chain of 6 to 10 consecutive atoms, by a chain of 11 to 15 consecutive atomes, by a chain of 16 to 20 consecutive atoms, by a chain of 21 to 25 consecutive atomes, by a chain of 26 to 30 consecutive atomes, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, linker L is a chain of 5 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 7 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 10 to 500 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X. In certain embodiments, linker L is a chain of 15 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

In certain embodiments, linker L is a chain of 5 to 500 consecutive atoms separating X and Y (or Z) and which comprises an optionally substituted arylene linked to X or optionally substituted heteroarylene linked to X. In certain embodiments, linker L is a chain of 7 to 500 consecutive atoms separating X and Y (or Z) and which comprises an optionally substituted arylene linked to X or optionally substituted heteroarylene linked to X. In certain embodiments, linker L is a chain of 10 to 500 consecutive atoms separating X and Y (or Z) and which comprises an optionally substituted arylene linked to X or optionally substituted heteroarylene linked to X. In certain embodiments, linker L is a chain of 15 to 400 consecutive atoms separating X and Y (or Z) and which comprises an optionally substituted arylene linked to X or optionally substituted heteroarylene linked to X.

In certain embodiments, linker L is a chain of 5 to 500 consecutive atoms separating X and Y (or Z) and which comprises an optionally substituted phenylene linked to X. In certain embodiments, linker L is a chain of 7 to 500 consecutive atoms separating X and Y (or Z) and which comprises an optionally substituted phenylene linked to X. In certain embodiments, linker L is a chain of 10 to 500 consecutive atoms separating X and Y (or Z) and which comprises an optionally substituted phenylene linked to X. In certain embodiments, linker L is a chain of 15 to 400 consecutive atoms separating X and Y (or Z) and which comprises an optionally phenylene linked to X.

In certain embodiments, linker L is a chain of 16 to 400 consecutive atoms separating X and Y (or Z) and which comprises an optionally substituted arylene linked to X, optionally substituted heteroarylene linked to X, optionally substituted heterocyclene linked to X, or optionally substituted cycloalkylene linked to X.

It is understood that the linker may be considered as connecting directly to a Z² group of a M6PR binding moiety (X) (e.g., as described herein). In some embodiments of formula (XI), the linker may be may be considered as connecting directly to the Z³ group. Alternatively, the —Ar—Z³— group of formula (XI) (e.g., as described herein) can be considered part of a linking moiety that connects Z² to Y. The disclosure is meant to include all such configurations of M6PR binding moiety (X) and linker (L).

In some embodiments of formula (I)-(Ia), L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa)

each L¹ to L⁷ is independently a linking moiety;

a is 1 or 2;

b, c, d, e, f, and g are each independently 0, 1, or 2; and

n is 1 to 500.

In some embodiments of formula (IIa), n is an integer of 1 to 5; wherein when d is 0, n is 1, when d is 1, n is an integer of 1 to 3, and when d is 2, n is an integer of 1 to 5.

In some embodiments of formula (IIa), L¹ comprises an optionally substituted aryl or heteroaryl group or linking moiety, e.g., as described in formula (XI). In some embodiments of formula (IIa), L¹ comprises a monocyclic or bicyclic or tricyclic aryl or heteroaryl group that is optionally substituted (e.g., as described herein). In some embodiments of formula (IIa), L¹ further comprises one or more linking moieties, each independently selected from a C₍₁₋₁₀₎alkyl, —O—, —S—, —NH—, —NHCO—, —CONH—, —NHC(═O)NH—, —NHC(═S)NH—, —NHCO₂—, —OC(═O)NH—, —OC(═O)—, —CO₂—, —(OCH₂)ₚ—, and —(OCH₂CH₂)ₚ—, where p is 1-20, such as 1-10, 1-6 or 1-3, e.g., 1 or 2.

In some embodiments of formula (IIa), each L¹ is independently

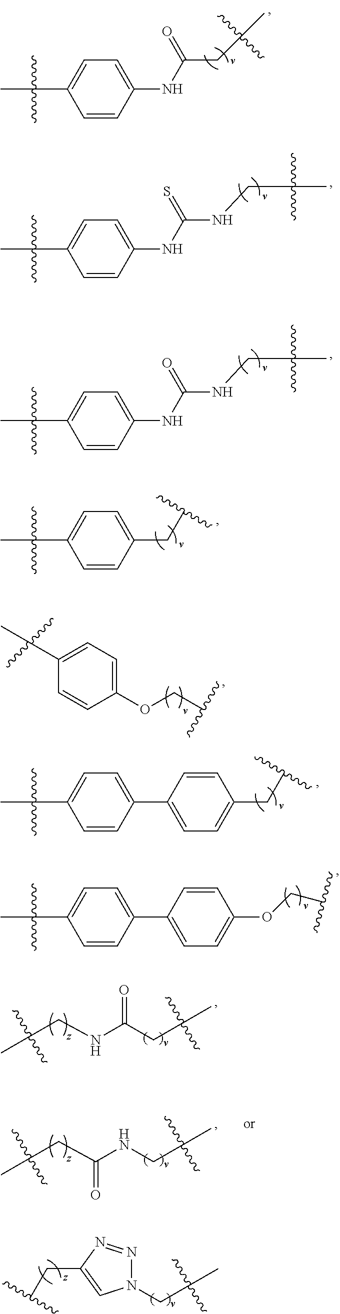

where z and v are independently 0-10, such as 0-6 or 0-3, e.g., 0, 1 or 2.

In certain embodiments of formula (IIa), L¹ is

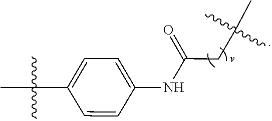

In certain embodiments of formula (IIa), L¹ is

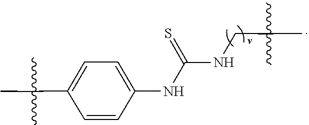

In certain embodiments of formula (IIa), L¹ is

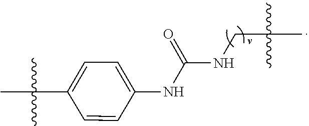

In certain embodiments of formula (IIa), L¹ is

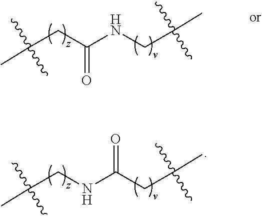

In certain embodiments of formula (IIa), L¹ is

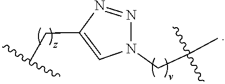

In certain embodiments of formula (IIa), each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—, where p is 1-20, such as 1-10, 1-6 or 1-3, e.g., 1 or 2.

In certain embodiments of formula (IIa), each L³ is independently

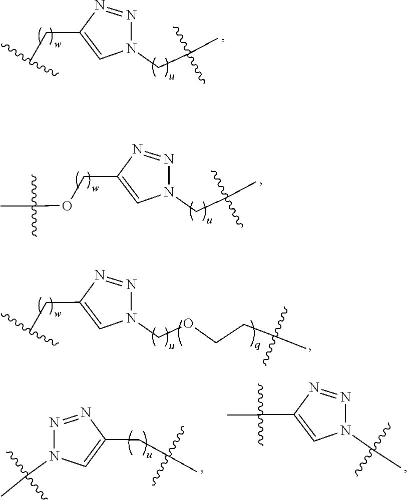

or —(OCH₂CH₂)_{q}—, where w and u are independently 0-10, such as 1-10, 1-6 or 1-3, e.g., 1 or 2, and q is 1-20 such as 1-10, 1-6 or 1-3, e.g., 1 or 2.

In some embodiments of formula (IIa), each L⁴ is a linear or branched linking moiety.

In some embodiments of formula (IIa), L⁴ is a branched linking moiety, e.g., a trivalent linking moiety. For example, an L⁴ linking moiety can be of the one of the following general formula:

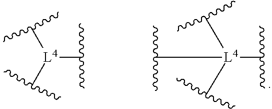

In some embodiments of formula (IIa), the branched linking moiety can be of higher valency and be described by one of the one of the following general formula:

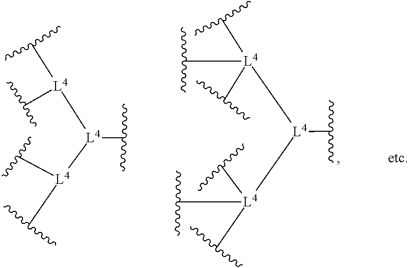

where any two L⁴ groups can be directed linked or connected via optional linear linking moieties (e.g., as described herein).

In some embodiments of formula (IIa), the branched linking moiety can include one, two or more L4 linking moieties, each being trivalent moieties, which when linked together can provide for multiple branching points for covalent attachment of the ligands and be described by one of the one of the following general formula:

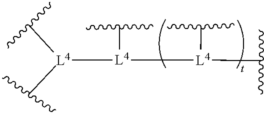

where t is 0 to 500, such as 0 to 100, 0 to 20, or 0 to 10.

In some embodiments, the branched linking moiety (e.g., L⁴) comprises one or more of: an amino acid residue (e.g., Asp, Lys, Orn, Glu), N-substituted amido (—N(—)C(═O)—), tertiary amino, polyol (e.g., O-substituted glycerol), and the like.

In some embodiments of formula (IIa), one or more L⁴ is selected from

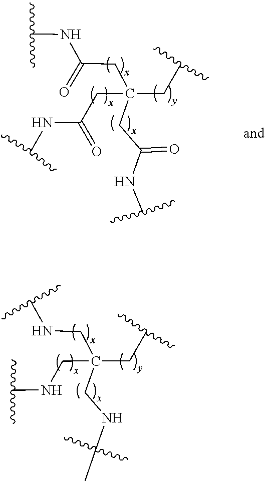

wherein each x and y is independently 1 to 20. In some cases, each x is 1, 2 or 3, e.g., 2.

In some embodiments of formula (IIa), each L⁴ is independently —OCH₂CH₂—,

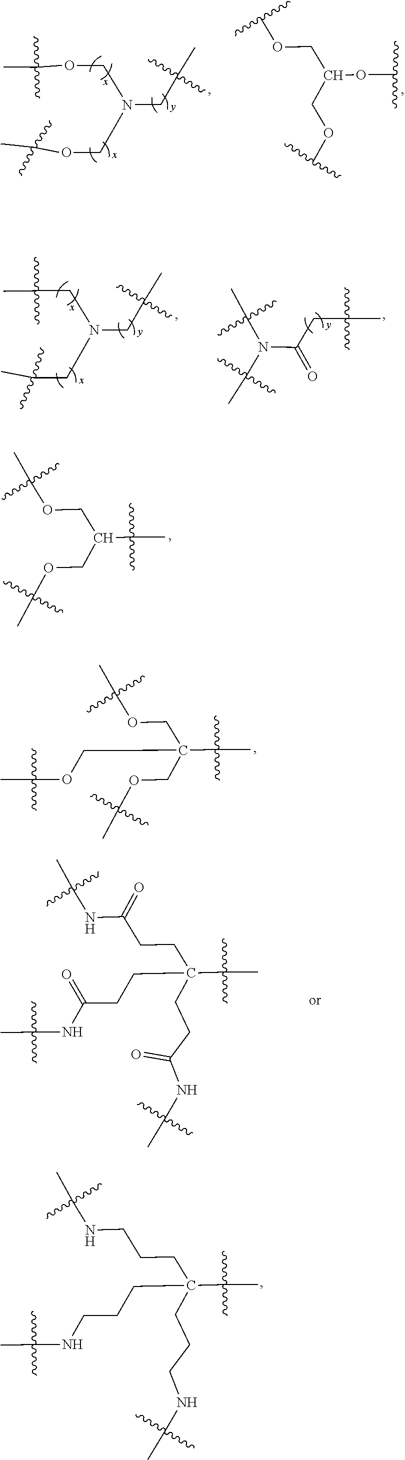

where each x and y are independently 1-10, such as 1-6 or 1-3, e.g., 1 or 2.

In some embodiments of formula (IIa), each L⁵ is independently —NHCO—C₁₋₆-alkylene-, —CONH—C₁₋₆-alkylene-, —C₁₋₆-alkylene-,

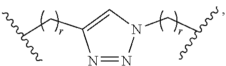

or —(OCH₂CH₂)_{f}—, where each r is independently 1-20, such as 1-10, 1-6 or 1-3, e.g., 1 or 2.

In some embodiments of formula (IIa), each L⁶ is independently —NHCO—C₁₋₆-alkylene-, —CONH—C₁₋₆-alkylene-, —C₁₋₆-alkylene-, or —(OCH₂CH₂), —, where s is 1-20, such as 1-10, 1-6 or 1-3, e.g., 1 or 2.

In some embodiments of formula (IIa), each L⁷ is independently —NHCO—C₁₋₆-alkylene-, —CONH—C₁₋₆-alkylene-, —C₁₋₆-alkylene-, —(OCH₂CH₂)ₜ—, or —OCH₂—, where t is 1-20, such as 1-10, 1-6 or 1-3, e.g., 1 or 2.

In some embodiments of formula (IIa):

each L¹ is independently

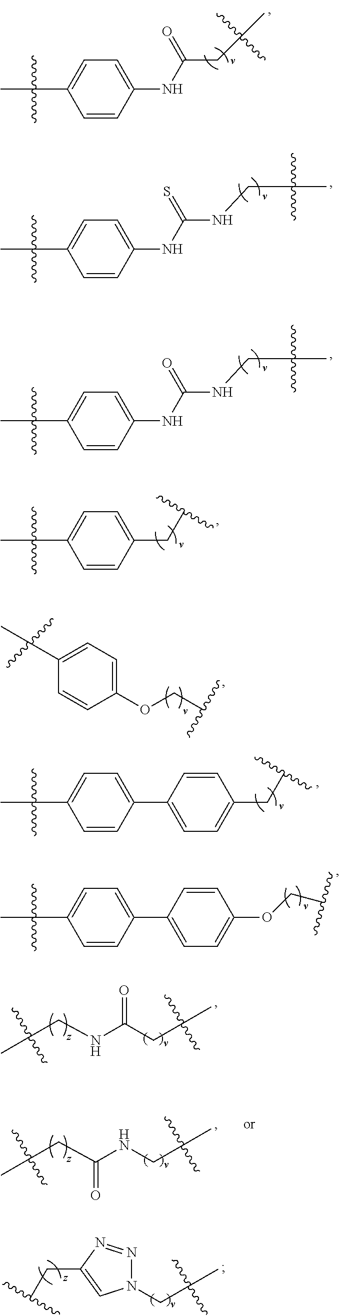

each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;

each L³ is independently

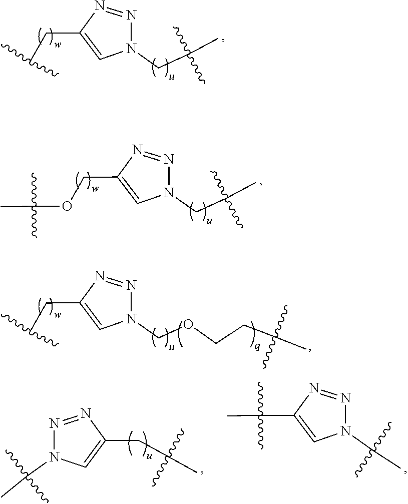

or —(OCH₂CH₂)_{q}—;

each L⁴ is independently —OCH₂CH₂—,

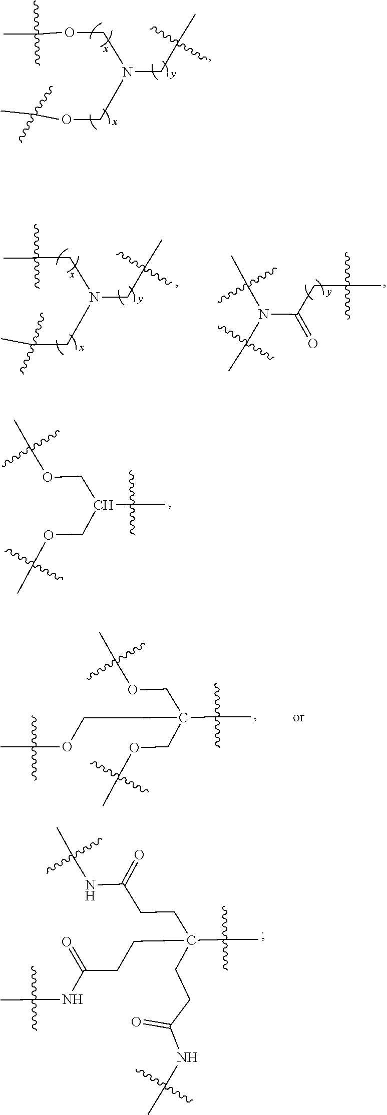

each L⁵ is independently —NHCO—C₁₋₆-alkylene-, —CONH—C₁₋₆-alkylene-, —C₁₋₆-alkylene-,

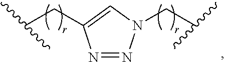

or —(OCH₂CH₂)ᵣ—;

each L⁶ is independently —NHCO—C₁₋₆-alkylene-, —CONH—C₁₋₆-alkylene-, —C₁₋₆-alkylene-, or —(OCH₂CH₂)ₛ—;

each each L⁷ is independently —NHCO—C₁₋₆-alkylene-, —CONH—C₁₋₆-alkylene-, —C₁₋₆-alkylene-, —(OCH₂CH₂)ₜ—, or —OCH₂—;

p, q, r, s, and t are each independently an integer of 1 to 20;

a is 1 or 2;

b, c, d, e, f, and g are each independently 0, 1, or 2;

u, v, w, x, y, and z are each independently an integer of 1 to 10; and

n is an integer of 1 to 5; wherein when d is O, n is 1, when d is 1, n is an integer of 1 to 3, and when d is 2, n is an integer of 1 to 5.

In some embodiments of formula (IIa):

each L¹ is independently

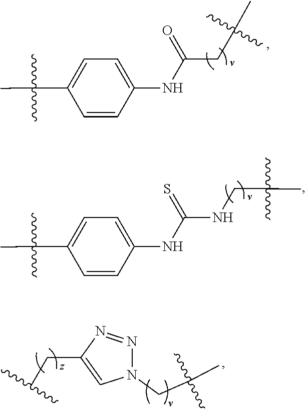

—C₁₋₆-alkylene-, —(OCH₂CH₂)ₖ—, or —(OCH₂CH₂)ₖ—(CH₂)ᵥ—;

each L² is independently —C₁₋₆-alkylene-, —NHCO—C₁₋₆-alkylene-, —(OCH₂)ₚ—, or —(OCH₂CH₂)ₚ—;

each L³ is independently

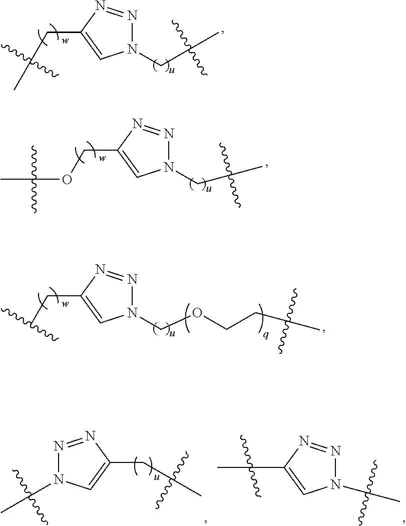

or —(OCH₂CH₂)_{q}—;

each L⁴ is independently —OCH₂CH₂—,

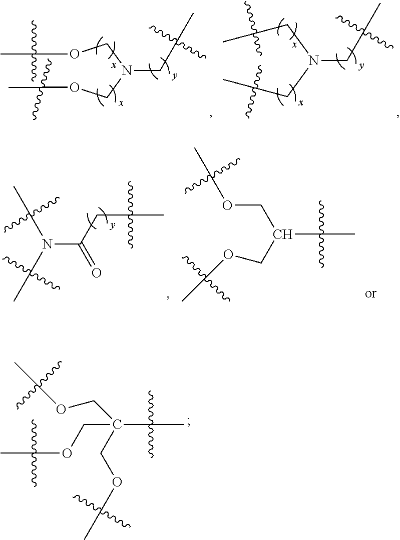

each L⁵ is independently —NHCO—C₁₋₆-alkylene- or —(OCH₂CH₂)ᵣ—;

each L⁶ is independently —NHCO—C₁₋₆-alkylene- or —(OCH₂CH₂)ₛ—;

each L⁷ is independently —NHCO—C₁₋₆-alkylene-, —(OCH₂CH₂)ₜ—, or —OCH₂—;

k, p, q, r, s, and t are each independently an integer of 1 to 20; a is 1 or 2; b, c, d, e, f, and g are each independently 0, 1, or 2; u, v, w, x, y, and z are each independently an integer of 1 to 10; and

n is an integer of 1 to 5; wherein when d is O, n is 1, when d is 1, n is an integer of 1 to 3, and when d is 2, n is an integer of 1 to 5.

In some embodiments of formula (IIa):

each L¹ is independently

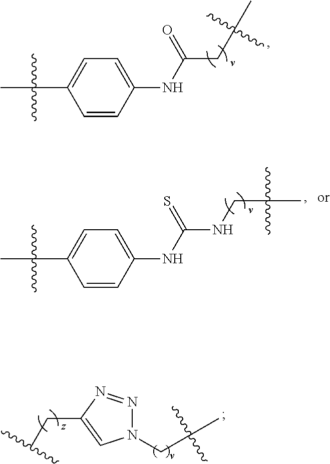

each L² is independently —C₁₋₆-alkylene-, —NHCO—C₁₋₆-alkylene-, —(OCH₂)ₚ—, or —(OCH₂CH₂)ₚ—;

each L³ is independently

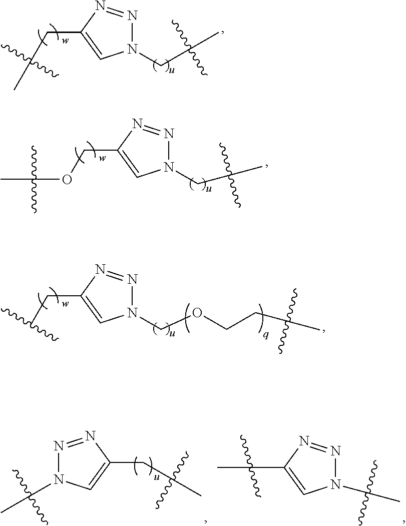

or —(OCH₂CH₂)_{q}—;

each L⁴ is independently —OCH₂CH₂—,

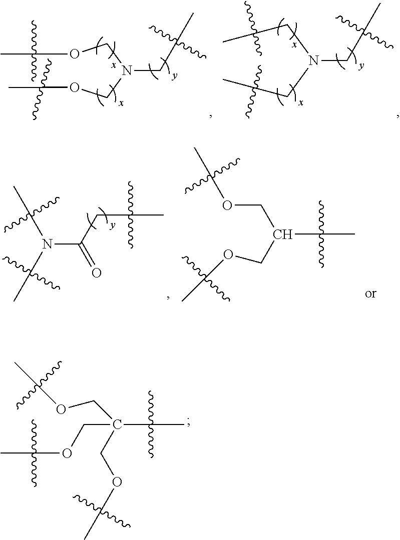

each L⁵ is independently —NHCO—C₁₋₆-alkylene- or —(OCH₂CH₂)ᵣ—;

each L⁶ is independently —NHCO—C₁₋₆-alkylene- or —(OCH₂CH₂)ₛ—;

each L⁷ is independently —NHCO—C₁₋₆-alkylene-, —(OCH₂CH₂)ₜ—, or —OCH₂—;

p, q, r, s, and t are each independently an integer of 1 to 20; a is 1 or 2; b, c, d, e, f, and g are each independently 0, 1, or 2; u, v, w, x, y, and z are each independently an integer of 1 to 10; and

n is an integer of 1 to 5; wherein when d is O, n is 1, when d is 1, n is an integer of 1 to 3, and when d is 2, n is an integer of 1 to 5.

In certain embodiments of formula (IIa), a is 1. In certain embodiments of formula (IIa), a is 1, and b, c, d, e, f, and g are 0.

In certain embodiments of formula (IIa), at least one of b, c, e, f, and g is not 0. In certain embodiments of formula (IIa), a, b, c and d are 1 and e, f and g are 0. In certain embodiments of formula (IIa), a, b, c, d and g are 1 and e and f are 0. In certain embodiments of formula (IIa), a, b, d, e and f are 1; c and g are 0; z is an integer from 2 to 10 and n is an integer of 1 to 5.

In certain embodiments of formula (IIa), at least one of b or c is not 0 and at least one of e, f, and g is not 0. In certain embodiments of formula (IIa), a, b, c, d, e and f are 1 and g is 0 or 1. In certain embodiments of formula (IIa), a, b, c, d, e, f and g are 1.

In certain embodiments of formula (IIa), a, b, and c are each independently 1 or 2.

In certain embodiments, k, p, q, r, s, and t are each independently an integer of 1 to 20. In certain embodiments, k, p, q, r, s, and t are each independently an integer of 1 to 10. In certain embodiments, k, p, q, r, s, and t are each independently an integer of 1 to 5. In certain embodiments, k, p, q, r, s, and t are each independently an integer of 1 to 3.

In certain embodiments, p, q, r, s, and t are each independently an integer of 1 to 20. In certain embodiments, p, q, r, s, and t are each independently an integer of 1 to 10. In certain embodiments, p, q, r, s, and t are each independently an integer of 1 to 5. In certain embodiments, p, q, r, s, and t are each independently an integer of 1 to 3.

In certain embodiments, u, v, w, x, y, and z are each independently an integer of 1 to 10. In certain embodiments, u, v, w, x, y, and z are each independently an integer of 1 to 5. In certain embodiments, u, v, w, x, y, and z are each independently an integer of 1 to 3.

In certain embodiments of formula (IIa), n is 1. In certain embodiments of formula (IIa), n is 2. In certain embodiments of formula (IIa), n is 3. In certain embodiments of formula (IIa), n is 4. In certain embodiments of formula (IIa), n is 5.

In yet another aspect, provided herein are compounds of formula (Ia) or (IIa), wherein L is a linker of the following formula (IIe):

-[(L¹)-(L²)-(L³)]ₙ-(L⁴)-(L⁵)- (IIe),

wherein L¹, L², L³, L⁴, L⁵, and n are as defined herein.

In certain embodiments of formula (IIe), L¹ is

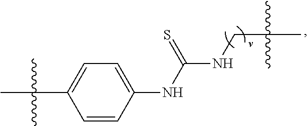

### L³ is

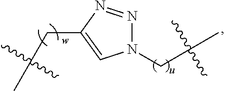

a is 1, b is 0, c is 1, u is 2, and the sum of v and w is 4.

In certain embodiments of formula (IIe), L¹ is

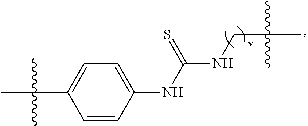

L² is methylene, is L³ is

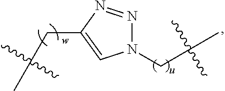

a is 1, b is 1, c is 1, u is 2, and the sum of v and w is 3.

In certain embodiments of formula (IIe), L¹ is

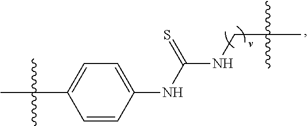

L² is methylene, L³ is

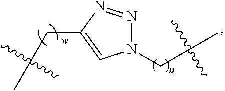

a is 1, b is 2, c is 1, u is 2, v is 1, and w is 1.

In certain embodiments of formula (IIe), L¹ is

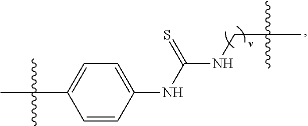

L² is ethylene, L³ is

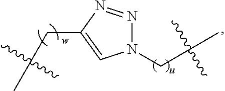

a is 1, b is 1, c is 1, u is 2, v is 1, and w is 1.

In certain embodiments of formula (IIe), L¹ is

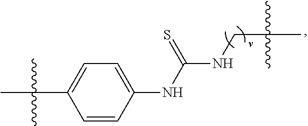

L² is methylene, L³ is

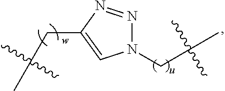

a is 1, b is 2, c is 1, u is 2, v is 1, and w is 1.

In certain embodiments of formula (IIe), L¹ is

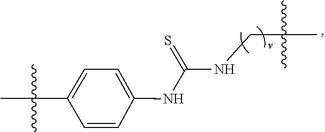

### L³ is

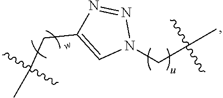

L⁵ is —(OCH₂CH₂)ᵣ—, a is 1, b is 0, c is 1, d is 0, u is 2, e is 1, and f and g are 0.

In certain embodiments of formula (IIe), L¹ is

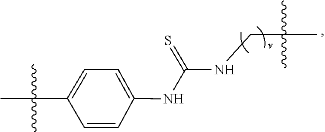

### L³ is

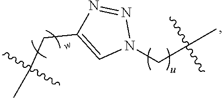

L⁵ is —(OCH₂CH₂)ᵣ—, a is 1, b is 0, c is 1, d is 1, u is 2, e is 1, f and g are 0, n is 1.

In certain embodiments of formula (IIe), L¹ is

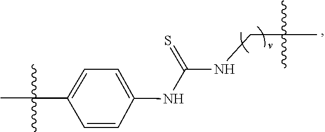

### L³ is

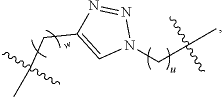

L⁵ is —(OCH₂CH₂)ᵣ—, a is 1, b is 0, c is 1, d is 1, u is 2, e is 1, f and g are 0, n is 2.

In certain embodiments of formula (IIe), L¹ is

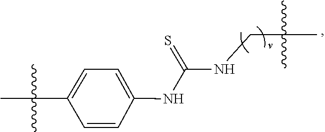

### L³ is

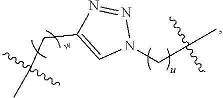

L⁵ is —(OCH₂CH₂)ᵣ—, a is 1, b is 0, c is 1, d is 1, u is 2, e is 1, f and g are 0, n is 3.

In certain embodiments of formula (IIe), L¹ is

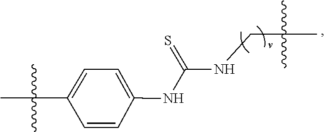

### L³ is

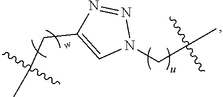

L⁵ is —(OCH₂CH₂), —, a is 1, b is 0, c is 1, d is 1, u is 2, the sum of v and w is 4, and n is 1, 2, or 3.

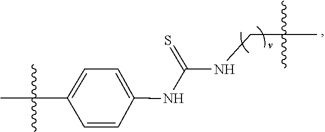

In certain embodiments of formula (IIe), L¹ is L² is methylene, is L³ is

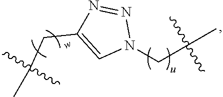

L⁵ is —(OCH₂CH₂)ᵣ—, a is 1, b is 1, c is 1, u is 2, the sum of v and w is 3, and n is 1, 2, or 3.

In certain embodiments of formula (IIe), L³ is

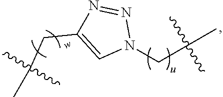

L⁵ is —(OCH₂CH₂)ᵣ—, a is 1, b is 0, c is 1, d is 1, u is 2, the sum of v and w is 4, and n is 1, 2, or 3.

In certain embodiments of formula (IIe), L² is methylene, is L³ is

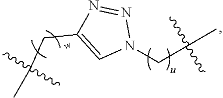

L⁵ is —(OCH₂CH₂)ᵣ—, a is 1, b is 1, c is 1, u is 2, the sum of v and w is 3, and n is 1, 2, or 3.

In another aspect, provided herein are compounds of the following formula (Ib):

X—Y (Ib);

or a salt, a single stereoisomer, a mixture of stereoisomers or an isotopic form thereof, wherein:

X is a moiety that binds to a cell surface mannose-6-phosphate receptor (M6PR); and

Y is a moiety having a structure of

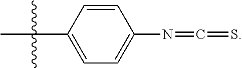 [SMILES: *c1ccc(N=C=S)cc1]

In another aspect, provided herein are compounds of formula (Ia), wherein L is a linker of the following formula (IIb):

-(L¹)ₐ-(L²)_{b}-(L³)_{c}- (IIb); and

wherein

L¹ is

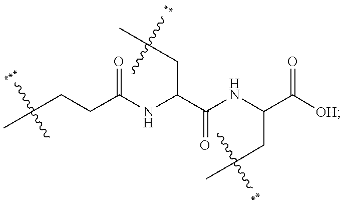 [SMILES: *CCC(=O)NC(C*)C(=O)NC(C*)C(=O)O]

L² is —(OCH₂CH₂)ₚ—;

L³ is —NHCO—C₁₋₆-alkylene-;

p is an integer of 1 to 20; a is 1, and b and c are each independently 0 or 1; n is 2;
wherein [symbol] represents the point of attachment to X, and [symbol] represents the point of attachment to L².

In some embodiments, Y is a chemoselective ligation group (e.g., an active ester, maleimide or isothiocyanate). In some embodiments, L¹ is

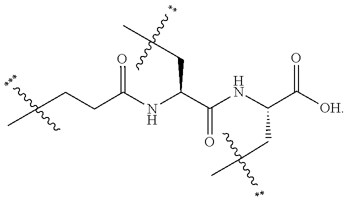 [SMILES: *CCC(=O)N[C@@H](C*)C(=O)N[C@@H](C*)C(=O)O]

In another aspect, provided herein are compounds of formula (Ia), wherein L is a linker of the following formula (IIc):

-(L¹)ₐ-(L²)_{b}-(L³)_{c}-(L⁴)_{d}- (IIc); and

wherein

L¹ is

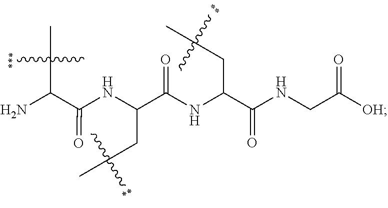 [SMILES: *CC(NC(=O)C(*)N)C(=O)NC(C*)C(=O)NCC(=O)O]

L² is

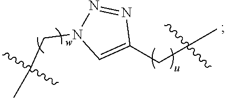

L³ is —NHCO—C₁₋₆-alkylene- or —(OCH₂CH₂)ₚ—;

L⁴ is —NHCO—C₁₋₆-alkylene- or —(OCH₂CH₂)_{q}—;

p and q are each independently an integer of 1 to 20; a is 1, and b, c, and d are each independently 0 or 1; and w and u are each independently an integer of 1 to 10;

wherein [symbol] represents the point of attachment to X, and [symbol] represents the point of attachment to L²; and n is 2.

In some embodiments, Y is a chemoselective ligation group (e.g., an active ester, maleimide or isothiocyanate). In some embodiments, L¹ is

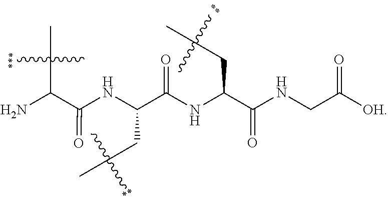 [SMILES: *C[C@H](NC(=O)C(*)N)C(=O)N[C@@H](C*)C(=O)NCC(=O)O]

In another aspect, provided herein are compounds of formula (Ia), wherein L is a linker of the following formula (IId):

[symbol]

(L¹)ₐ-(L²)_{b} [symbol] ₙ(L³)_{c}-(L⁴)_{d}- (IId); and

wherein

L¹ is

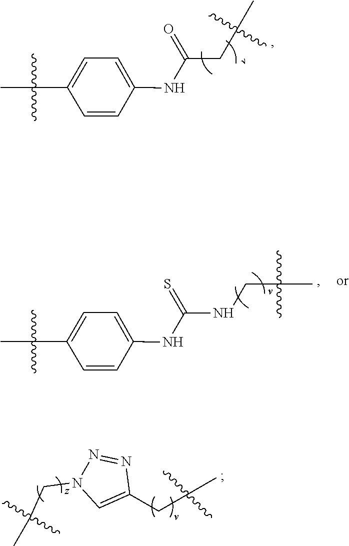

L² is

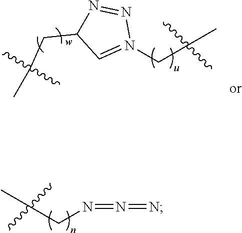

L³ is

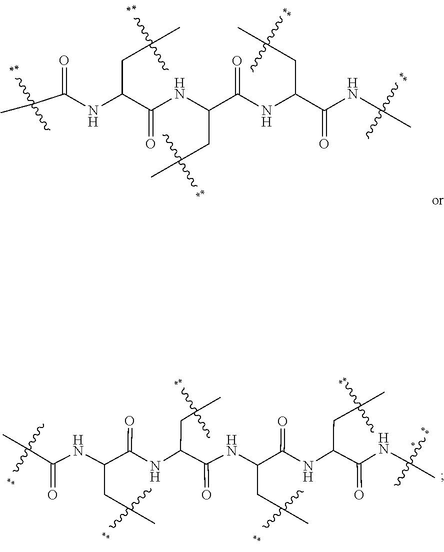 [SMILES: *CC(NC(*)=O)C(=O)NC(C*)C(=O)NC(C*)C(=O)N* ; *CC(NC(*)=O)C(=O)NC(C*)C(=O)NC(C*)C(=O)NC(C*)C(=O)N*]

L⁴ is —CH₂CH₂(OCH₂CH₂)_{q}—;

p is an integer of 1 to 20; c is 1, and a, b, and d are each independently 0 or 1; and u, v, w, and z are each independently an integer of 1 to 10;

wherein [symbol] represents the point of attachment to an H or L², and [symbol] represents the point of attachment to L⁴; and

n is an integer of 1 to 5.

In some embodiments formula (IId), Y is a chemoselective ligation group.

In certain embodiments formula (IId), L³ is

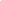

In certain embodiments formula (IId), X is of formula (IIIa), (IIIb), (IIIc), or (IIId), e.g., as described herein. In certain embodiments formula (IId), X is formula (IIIa′), (IIIa″), (IIIb′), (IIIb″), (IIIc′), (IIIc″), (IIId′) or (IIId″), e.g., as described herein. In certain embodiments, X is of formula (IIIa′), (IIIa″), (IIIb′), or (IIIb″). In certain embodiments formula (IId), X is of formula (IIIc′), (IIIc″), (IIId′) or (IIId″). In certain embodiments formula (IId), X is of formula (IIIa′) or (IIIa″). In certain embodiments formula (IId), X is of formula (IIIb′) or (IIIb″). In certain embodiments formula (IId), X is of formula (IIIc′) or (IIIc″). In certain embodiments formula (IId), X is of formula (IIId′) or (IIId″). In certain embodiments formula (IId), X is of formula (IIIa). In one embodiment formula (IId), X is of formula (IIIa″). In certain embodiments formula (IId), X is of formula (IIIb′). In one embodiment formula (IId), X is of formula (IIIb″). In certain embodiments formula (IId), X is of formula (IIIc′). In one embodiment formula (IId), X is of formula (IIIc″). In certain embodiments formula (IId), X is of formula (IIId′). In one embodiment formula (IId), X is of formula (IIId″). In certain embodiments formula (IId), X is of formula (IIIe). In one embodiment, j is 1 or 2. In another embodiment, j is 2 or 3. In another embodiment, j is 1. In another embodiment, j is 2. In yet another embodiment, j is 3.

In certain embodiments of formula (IId), X (e.g., as described above) includes a hydrophilic head group (e.g., R″) that is as described in any one of the embodiments described herein. In certain embodiments of formula (IId), the X includes an R″ group that is selected from the group consisting of —OH, —CR¹R²OH, —P═O(OH)₂, P(═O)R¹OH, —(CR¹R²)—P═O(OH)₂, —SO₂OH, —OSO₂OH, —COOH, —CONH₂, —CONHR¹, —CONR³R⁴, —CONHSO₂R³, —CH(COOH)₂, —CR¹R²COOH, —SO₂R³, —SOR³R⁴, —SO₂NH₂, —SO₂NHR³, —SO₂NR³R⁴, —NHCOR³, —NHSO₂R³,

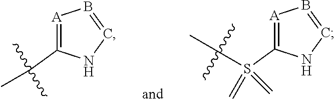

wherein R¹ and R² are each independently hydrogen, halo, or CN;
wherein R³ and R⁴ are each independently C₁₋₆ alkyl; and
wherein A, B, and C are each independently CH or N.

In certain embodiments, R″ is selected from the group consisting of —P═O(OH)₂, P(═O)R¹OH, and —(CR¹R²)—P═O(OH)₂. In certain embodiments, R″ is selected from the group consisting of —SO₂OH, —OSO₂OH, —CONHSO₂R³, —SO₂R³, —SOR³R⁴, —SO₂NH₂, —SO₂NHR³, —SO₂NR³R⁴, and —NHSO₂R³. In certain embodiments, R″ is —OH, or —CR¹R²OH In certain embodiments, R″ is selected from the group consisting of —COOH, —CONH₂, —CONHR¹, —CONR³R⁴, —CH(COOH)₂, —CR¹R²COOH, and —NHCOR³.

Tables 2-3 shows a variety of exemplary linkers or linking moieties that find use in the compounds described herein. In some embodiments of formula (I)-(IIe) or (XI)-(XVIa), the compound includes any one of the linkers or linking moieties set forth in Tables 2-3.

TABLE 2
Exemplary linear linkers and linking moieties

| Linker No. | Linker structure |
|---|---|
| 1 | 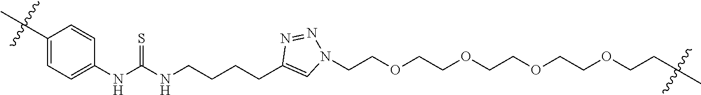 [SMILES: *CCOCCOCCOCCOCCn1cc(CCCCNC(=S)Nc2ccc(*)cc2)nn1] |
| 2 | 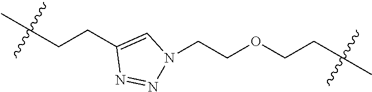 [SMILES: *CCOCCn1cc(CC*)nn1] |
| 3 | 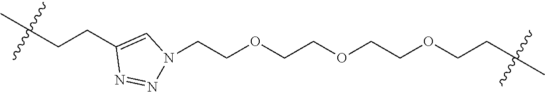 [SMILES: *CCOCCOCCOCCn1cc(CC*)nn1] |
| 4 | 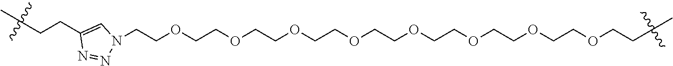 [SMILES: *CCOCCOCCOCCOCCOCCOCCOCCOCCn1cc(CC*)nn1] |
| 5 | 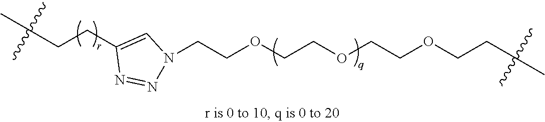 |
| 6 | 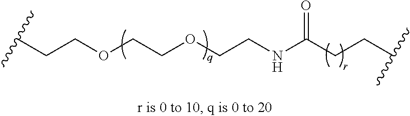 |
| 7 | 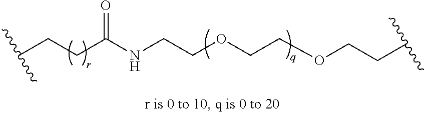 |
| 8 | 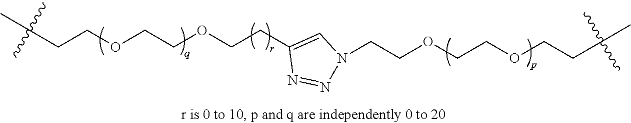 |
| 9 | 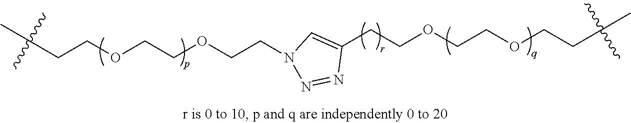 |
| 10 | 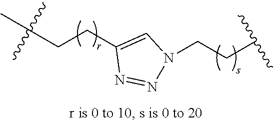 |

TABLE 3
Exemplary branched linkers and branched linking moieties

| Linker | |
|---|---|
| No. | Linker structure |
| 11 | 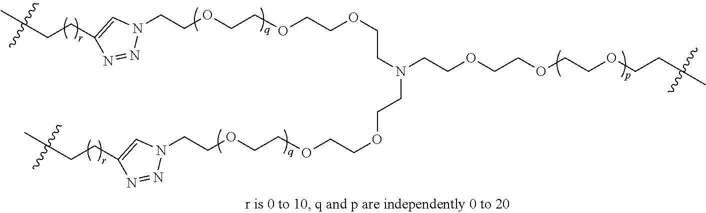 |
| 12 | 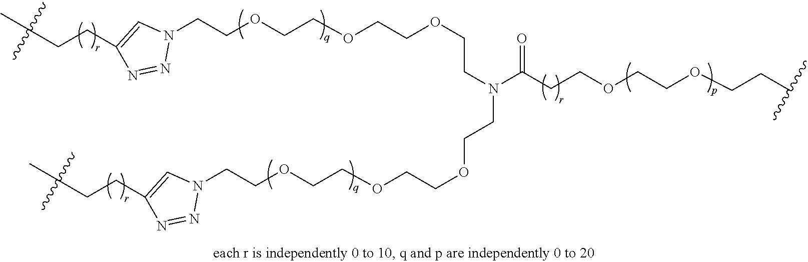 |
| 13 | 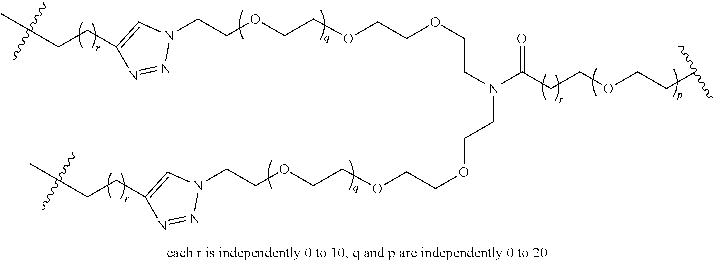 |
| 14 | 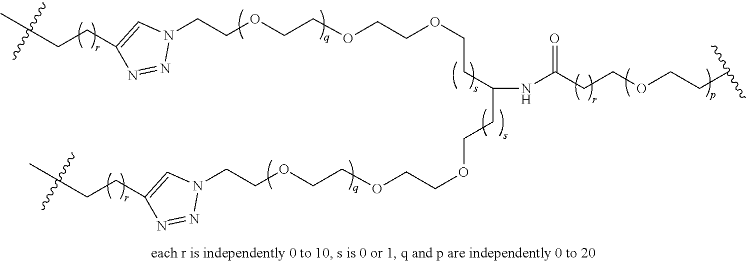 |
| 15 | 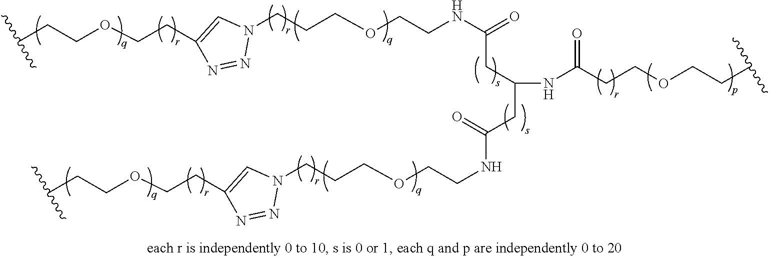 |
| 16 | 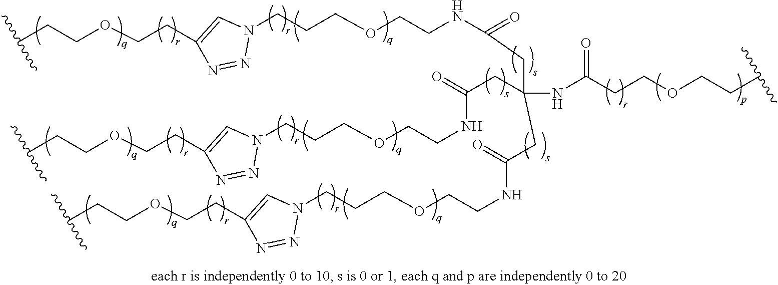 |
| 17 | 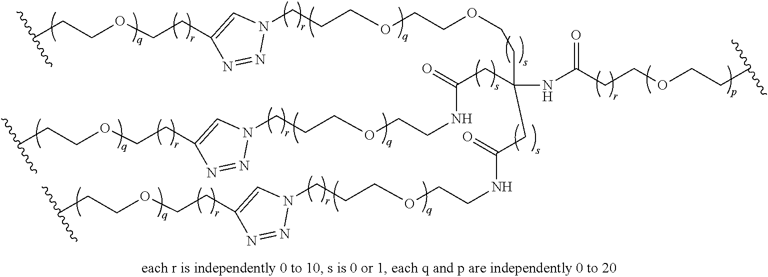 |

### Moiety of Interest (Y)

As summarized above, the M6PR or ASGPR binding compounds of this disclosure generally include a linked moiety of interest Y. In some embodiments, the moiety of interest Y is a chemoselective ligation group or a precursor thereof, and the compound can find use in the preparation of a variety of conjugates via conjugation of the chemoselective ligation group to a compatible reactive group of another moiety of interest, e.g., as described herein.

### Chemoselective Ligation Groups

In certain embodiments of formula (I)-(XVIa), Y is a chemoselective ligation group, or a precursor thereof. A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, reductive amination, dialkyl squarate chemistry, etc.

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

In some instances, chemoselective ligation group is capable of spontaneous conjugation to a compatible chemical group when the two groups come into contact under sutiable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions).

In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

In some instances, Y is a precursor of the reactive functionality or function group capable of conjugation to a compatible group of a second moiety. For example, a carboxylic acid is a precursor of an active ester chemoselective ligation group.

In certain embodiments of formula (Ia)-(XVIa), Y is a reactive moiety capable forming a covalent bond to a polypeptide (e.g., with an amino acid sidechain of a polypeptide having a compatible reactive group). The reactive moiety can be referred to as a chemoselective ligation group.

In certain embodiments of formula (Ia)-(XVIa), Y is a thio-reactive chemoselective ligation group (e.g., as described in Table 4). In some cases, Y can produce a residual moiety Z resulting from the covalent linkage of a thiol-reactive chemoselective ligation group to one or more cysteine residue(s) of a protein, e.g., Ab.

In certain embodiments of formula (Ia)-(XVIa), Y is an amino-reactive chemoselective ligation group (e.g., as described in Table 4). In some cases, Y can produce a residual moiety Z resulting from the covalent linkage of an amine-reactive chemoselective ligation group to one or more lysine residue(s) a protein, e.g., Ab.

Exemplary chemoselective ligation groups, and synthetic precursors thereof, which may be adapted for use in the compounds of this disclosure are shown in Table 4.

TABLE 4
Exemplary chemoselective ligation groups and precurors

| Groups | Exemplary structures |
|---|---|
| carboxylic acid or active ester | 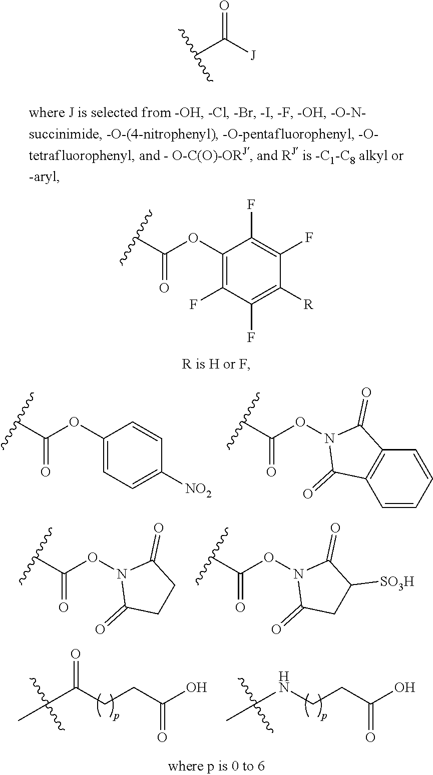 |
| maleimide | 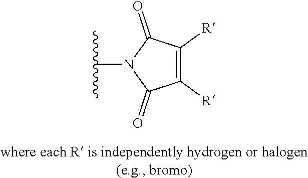 |
| isocyanate or isothiocyanate | 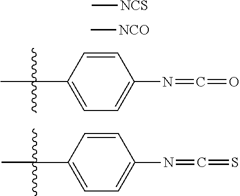 |
| alkyl halide alkyl tosylate | 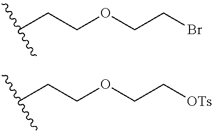 [SMILES: *CCOCCBr ; *CCOCCOS(=O)(=O)c1ccc(C)cc1] |
| aldehyde | 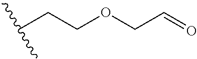 [SMILES: *CCOCC=O] |
| haloacetamide or alpha-leaving group acetamide | 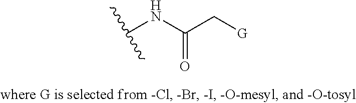 |
| 2-sulfonylpyridine | 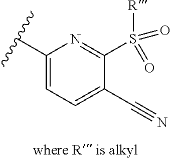 |
| diazirine | 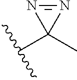 [SMILES: *C1(C)N=N1] |
| sulfonyl halide or vinyl sulfone | 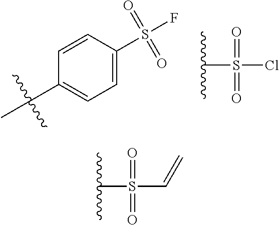 |
| hydrazide hydrazino hydroxylamino | 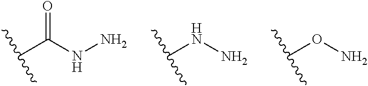 |
| pyridyl disulfide | 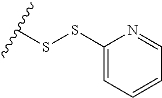 [SMILES: *SSc1ccccn1] |
| (HIPS) hydrazinyl- indolyl group, or (aza-HI PS) hydrazinyl- pyrrolo-pyridinyl group | 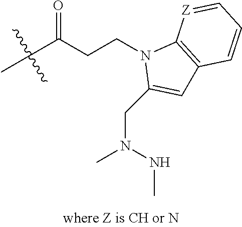 |
| alkyne or cyclooctyne | 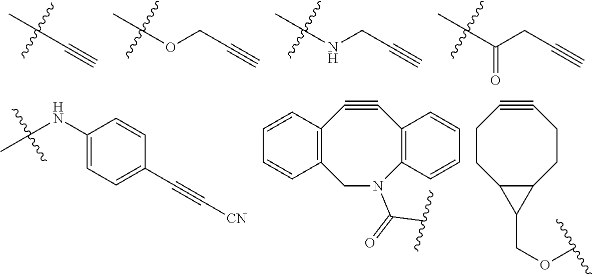 |
| azide | 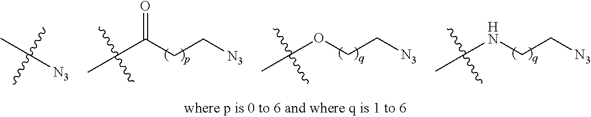 |
| amine | 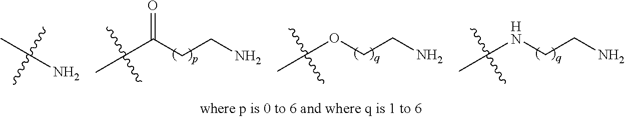 |

In Table 4, the [symbol] can represent a point of attachment of Y to a linking moiety or a linked X moiety.

Exemplary Compounds with Chemoselective Ligation Group

This disclosure includes compounds of formula (Ia)-(Ib) which can include:

(1) one or more particular M6PR ligand (X) (e.g., as described herein, such as ligands X1-X42 of Table 1) or a particular ASGPR ligand (X) (e.g., as described herein),
(2) a linker including one or more linking moieties (e.g., as described herein, such as any one or more of the linking moieties of Tables 2-3); and
(3) a chemoselective ligation group (Y) e.g., as described herein, such as any one of the groups of Table 4).

Tables 5-7B illustrate several exemplary M6PR binding compounds of this disclosure that include a chemoselective ligation group, or a precursor thereof. It is understood that this disclosure includes Y (e.g., as described herein) conjugates of each of the exemplary compounds of Tables 5-7B. For example, conjugates where the chemoselective ligation group has been conjugated to a different Y, such as a biomolecule or a small molecule ligand for a target protein.

Tables 8-9 illustrate several exemplary ASGPR binding compounds of this disclosure that include a chemoselective ligation group, or a precursor thereof. It is understood that this disclosure includes Y (e.g., as described herein) conjugates of each of the exemplary compounds of Tables 8-9. For example, conjugates where the chemoselective ligation group has been conjugated to a different Y, such as a biomolecule or a small molecule ligand for a target protein.

The chemoselective ligation group of such compounds can be utilized to connect to another Y moiety of interest (e.g., as described below). It is understood that any of these compounds can also be prepared de novo to include an alternative Y moiety of interest (e.g., as described below) rather than the chemoselective ligation group. In some embodiments, such compounds are referred to as a conjugate, e.g., a biomolecule conjugate that specifically binds a target protein.

TABLE 5
Exemplary M6PR binding compounds of Formula (XIa)

| # | Compound structure |
|---|---|
| 501 (I-1) | 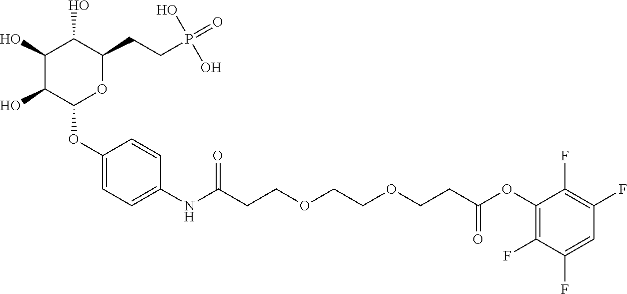 [SMILES: O=C(CCOCCOCCC(=O)Oc1c(F)c(F)cc(F)c1F)Nc1ccc(O[C@H]2O[C@H](CCP(=O)(O)O)[C@@H](O)[C@H](O)[C@@H]2O)cc1] |
| 502 | 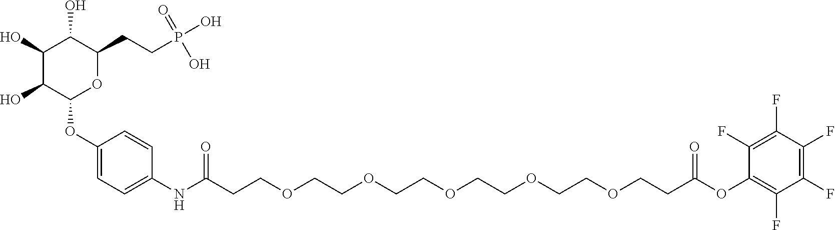 [SMILES: O=C(CCOCCOCCOCCOCCOCCC(=O)Oc1c(F)c(F)c(F)c(F)c1F)Nc1ccc(O[C@H]2O[C@H](CCP(=O)(O)O)[C@@H](O)[C@H](O)[C@@H]2O)cc1] |
| 503 | 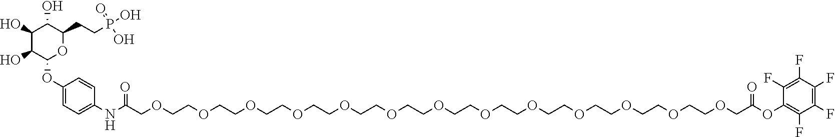 [SMILES: O=C(COCCOCCOCCOCCOCCOCCOCCOCCOCCOCCOCCOCCOCC(=O)Oc1c(F)c(F)c(F)c(F)c1F)Nc1ccc(O[C@H]2O[C@H](CCP(=O)(O)O)[C@@H](O)[C@H](O)[C@@H]2O)cc1] |
| 504 | 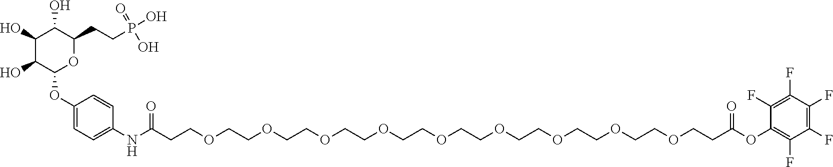 [SMILES: O=C(CCOCCOCCOCCOCCOCCOCCOCCOCCOCCC(=O)Oc1c(F)c(F)c(F)c(F)c1F)Nc1ccc(O[C@H]2O[C@H](CCP(=O)(O)O)[C@@H](O)[C@H](O)[C@@H]2O)cc1] |
| 505 (I-2) | 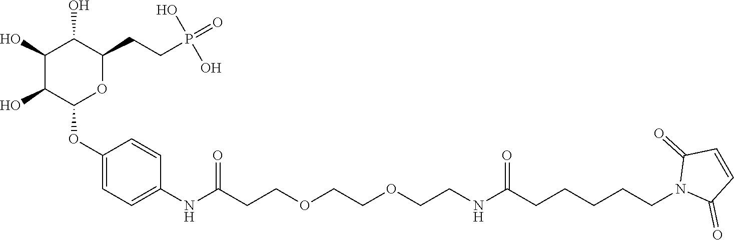 [SMILES: O=C(CCCCCN1C(=O)C=CC1=O)NCCOCCOCCC(=O)Nc1ccc(O[C@H]2O[C@H](CCP(=O)(O)O)[C@@H](O)[C@H](O)[C@@H]2O)cc1] |
| 506 | 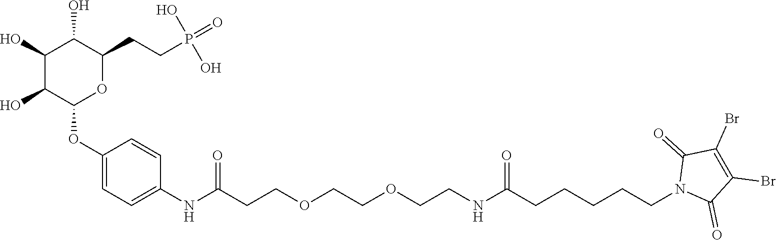 [SMILES: O=C(CCCCCN1C(=O)C(Br)=C(Br)C1=O)NCCOCCOCCC(=O)Nc1ccc(O[C@H]2O[C@H](CCP(=O)(O)O)[C@@H](O)[C@H](O)[C@@H]2O)cc1] |
| 507 | 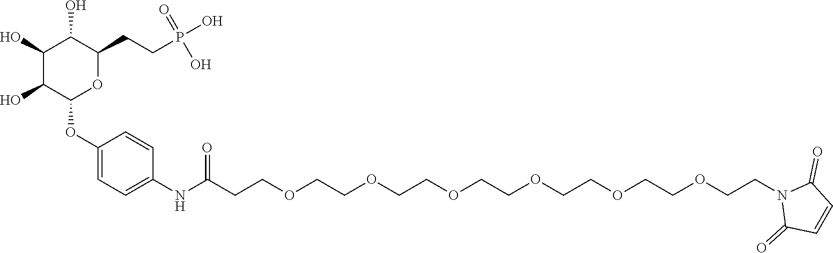 [SMILES: O=C(CCOCCOCCOCCOCCOCCOCCN1C(=O)C=CC1=O)Nc1ccc(O[C@H]2O[C@H](CCP(=O)(O)O)[C@@H](O)[C@H](O)[C@@H]2O)cc1] |
| 508 (I-3) | 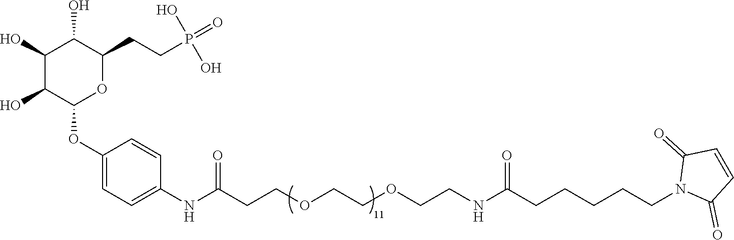 |
| 509 | 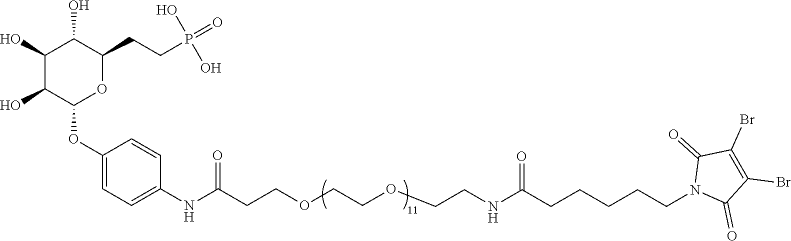 |
| 510 | 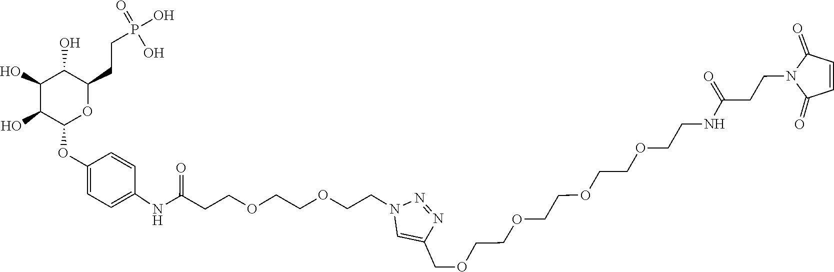 [SMILES: O=C(CCN1C(=O)C=CC1=O)NCCOCCOCCOCCOCc1cn(CCOCCOCCC(=O)Nc2ccc(O[C@H]3O[C@H](CCP(=O)(O)O)[C@@H](O)[C@H](O)[C@@H]3O)cc2)nn1] |
| 511 (I-4) | 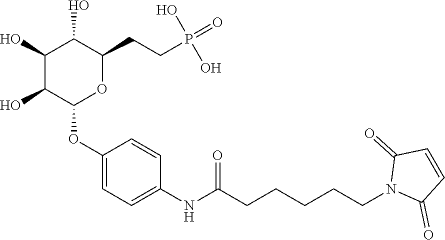 [SMILES: O=C(CCCCCN1C(=O)C=CC1=O)Nc1ccc(O[C@H]2O[C@H](CCP(=O)(O)O)[C@@H](O)[C@H](O)[C@@H]2O)cc1] |
| 512 (I-5) | 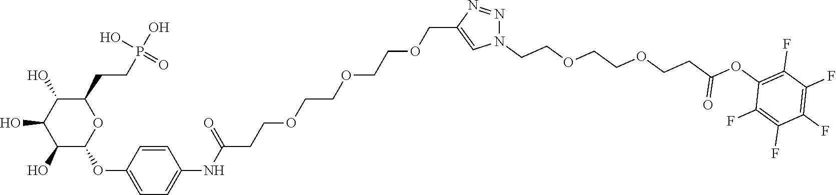 [SMILES: O=C(CCOCCOCCOCc1cn(CCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 513 (I-39) | 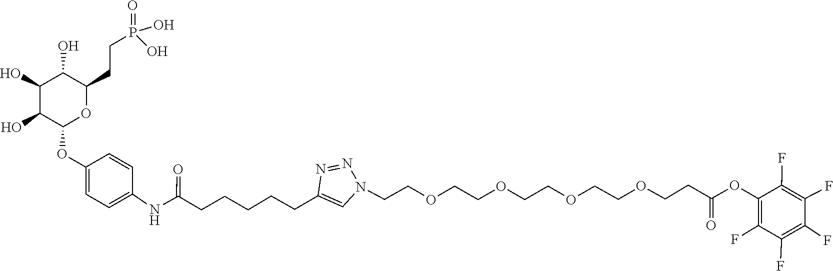 [SMILES: O=C(CCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 514 (I-57) | 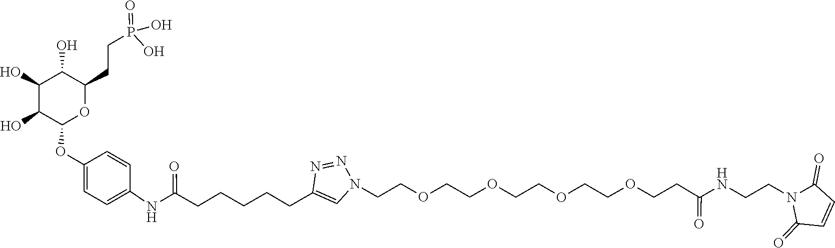 [SMILES: O=C(CCOCCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 515 (I-16) | 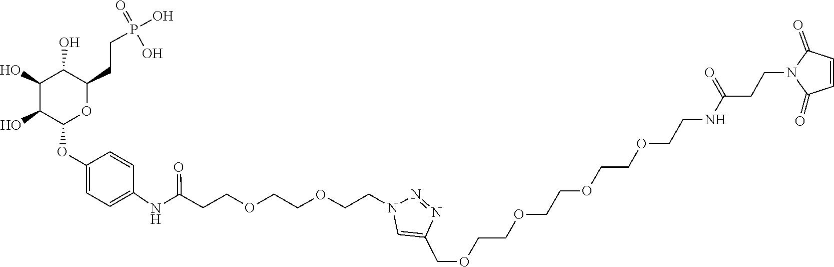 [SMILES: O=C(CCN1C(=O)C=CC1=O)NCCOCCOCCOCCOCc1cn(CCOCCOCCC(=O)Nc2ccc(O[C@H]3O[C@H](CCP(=O)(O)O)[C@@H](O)[C@H](O)[C@@H]3O)cc2)nn1] |
| 516 (I-6) | 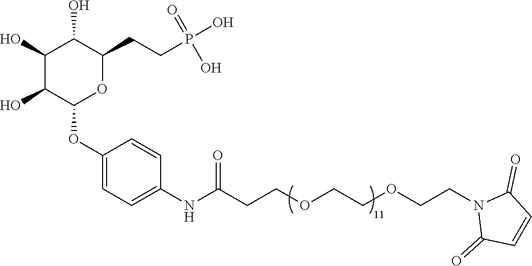 |
| 517 | 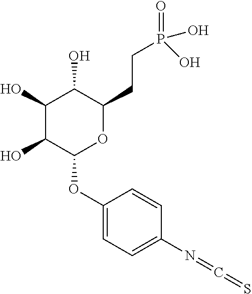 [SMILES: O=P(O)(O)CC[C@H]1O[C@H](Oc2ccc(N=C=S)cc2)[C@@H](O)[C@@H](O)[C@@H]1O] |
| 518 | 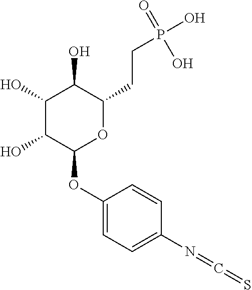 [SMILES: O=P(O)(O)CC[C@@H]1O[C@@H](Oc2ccc(N=C=S)cc2)[C@H](O)[C@H](O)[C@H]1O] |
| 519 (I-47) | 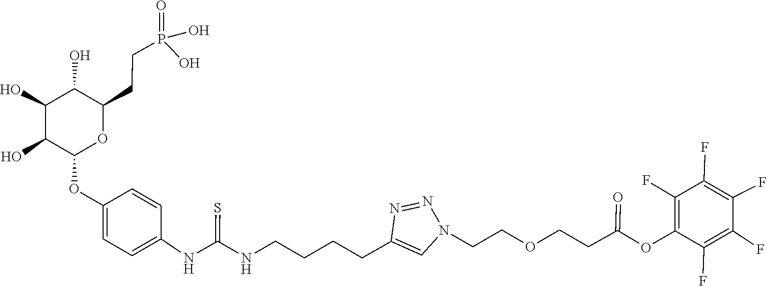 [SMILES: O=C(CCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 520 (I-7) | 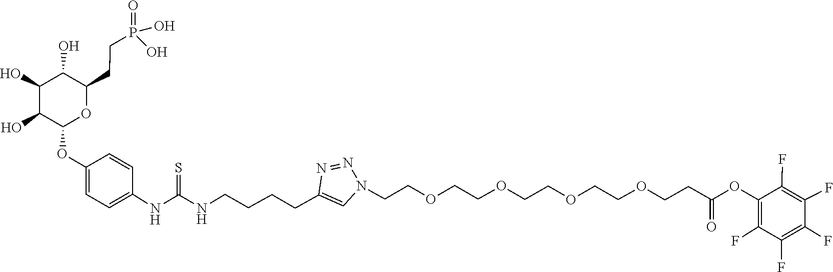 [SMILES: O=C(CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 521 | 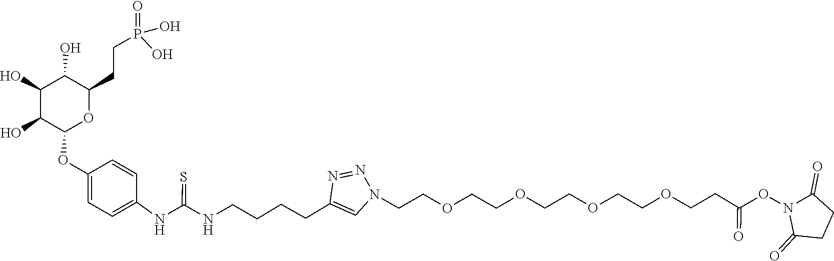 [SMILES: O=C(CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)ON1C(=O)CCC1=O] |
| 522 (I-49) | 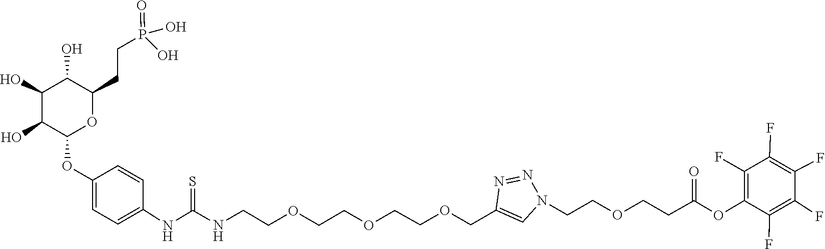 [SMILES: O=C(CCOCCn1cc(COCCOCCOCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 523 (I-17) | 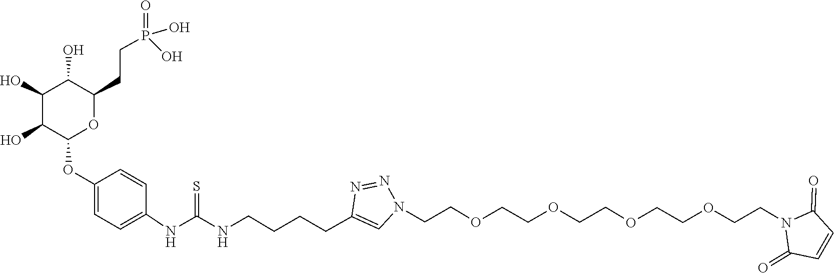 [SMILES: O=C1C=CC(=O)N1CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1] |
| 524 (I-18) | 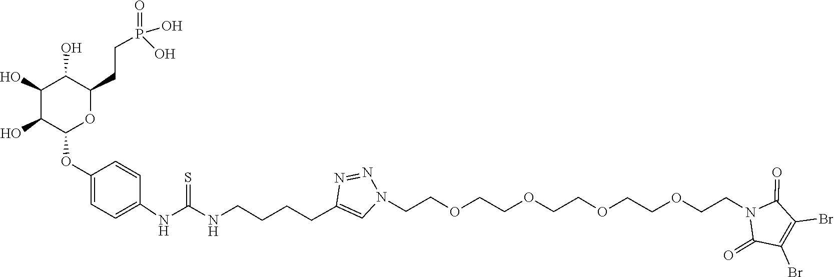 [SMILES: O=C1C(Br)=C(Br)C(=O)N1CCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1] |
| 525 | 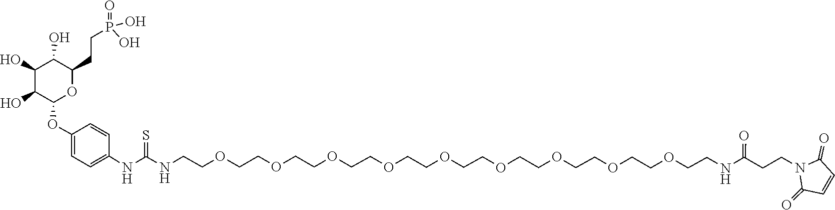 [SMILES: O=C(CCN1C(=O)C=CC1=O)NCCOCCOCCOCCOCCOCCOCCOCCOCCOCCNC(=S)Nc1ccc(O[C@H]2O[C@H](CCP(=O)(O)O)[C@@H](O)[C@H](O)[C@@H]2O)cc1] |
| 526 (I-48) | 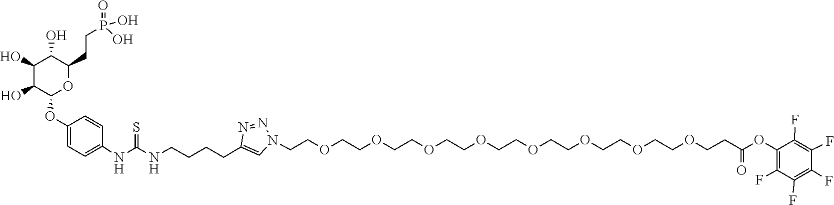 [SMILES: O=C(CCOCCOCCOCCOCCOCCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 527 | 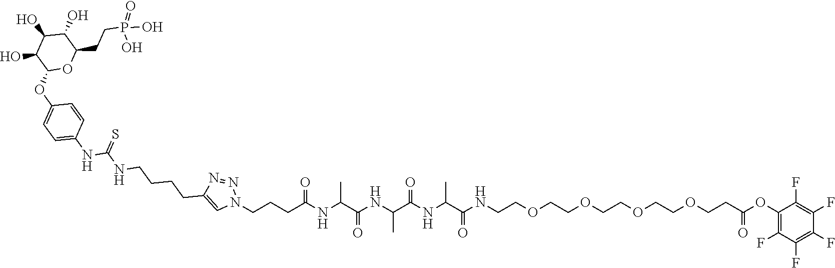 [SMILES: CC(NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(C)C(=O)NC(C)C(=O)NCCOCCOCCOCCOCCC(=O)Oc1c(F)c(F)c(F)c(F)c1F] |
| 528 (I-51) | 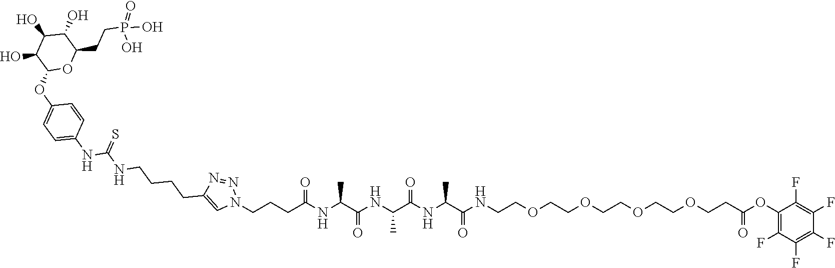 [SMILES: C[C@H](NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](C)C(=O)N[C@@H](C)C(=O)NCCOCCOCCOCCOCCC(=O)Oc1c(F)c(F)c(F)c(F)c1F] |
| 529 (I-38) | 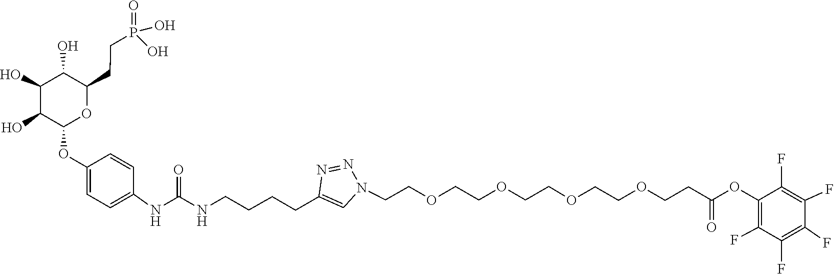 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1] |
| 530 (I-50) | 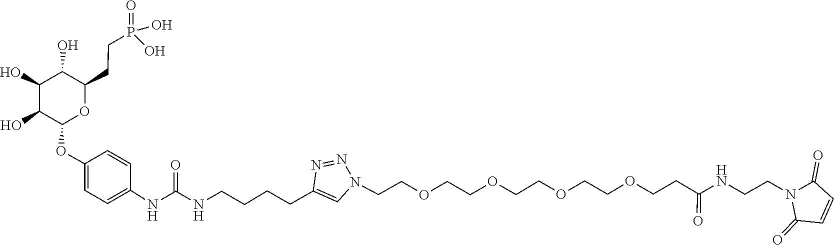 [SMILES: O=C(CCOCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 531 | 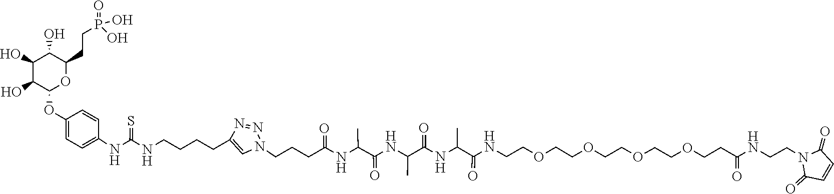 [SMILES: CC(NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(C)C(=O)NC(C)C(=O)NCCOCCOCCOCCOCCC(=O)NCCN1C(=O)C=CC1=O] |
| 532 (I-55) | 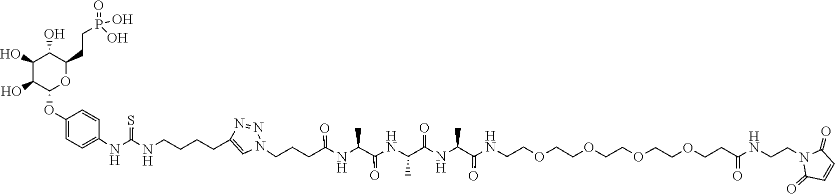 [SMILES: C[C@H](NC(=O)CCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](C)C(=O)N[C@@H](C)C(=O)NCCOCCOCCOCCOCCC(=O)NCCN1C(=O)C=CC1=O] |
| 533 (I-61) | 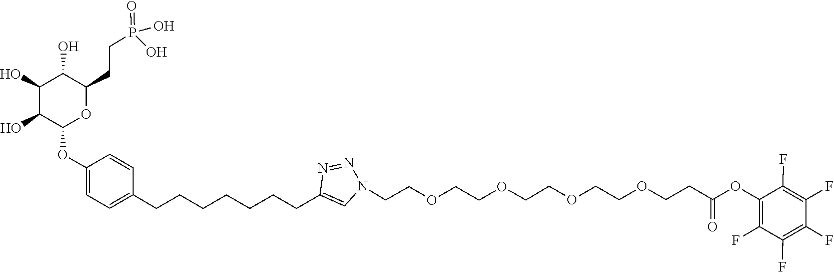 [SMILES: O=C(CCOCCOCCOCCOCCn1cc(CCCCCCCc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 534 (I-62) | 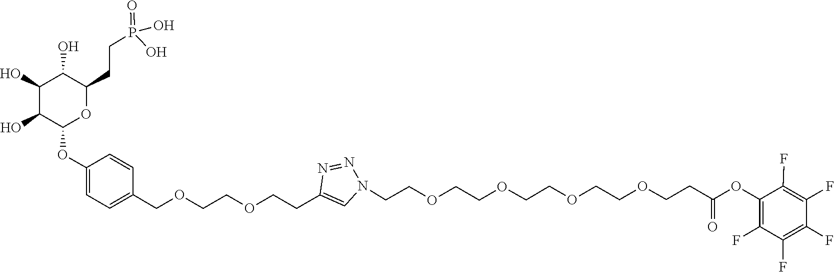 [SMILES: O=C(CCOCCOCCOCCOCCn1cc(CCOCCOCc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 535 (I-88) | 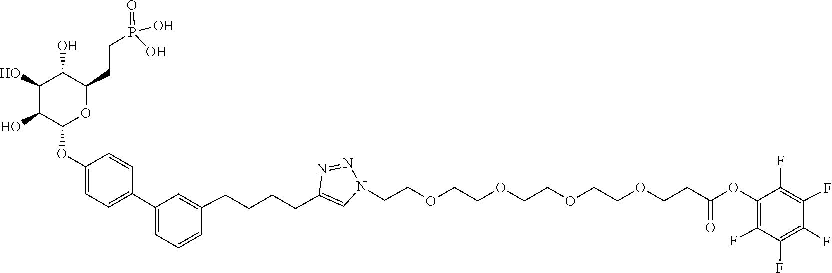 [SMILES: O=C(CCOCCOCCOCCOCCn1cc(CCCCc2cccc(-c3ccc(O[C@H]4O[C@H](CCP(=O)(O)O)[C@@H](O)[C@H](O)[C@@H]4O)cc3)c2)nn1)Oc1c(F)c(F)c(F)c(F)c1F] |
| 536 (I-60) | 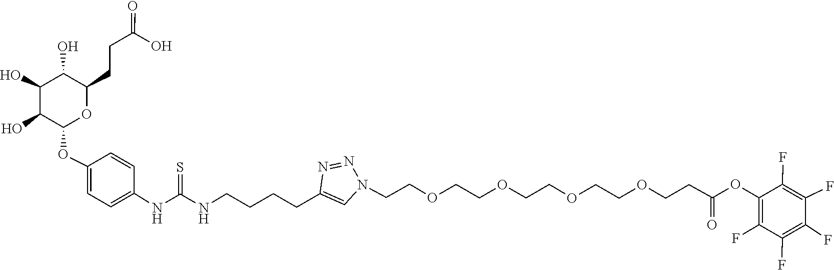 [SMILES: O=C(O)CC[C@H]1O[C@H](Oc2ccc(NC(=S)NCCCCc3cn(CCOCCOCCOCCOCCC(=O)Oc4c(F)c(F)c(F)c(F)c4F)nn3)cc2)[C@@H](O)[C@@H](O)[C@@H]1O] |
| 537 (I-66) | 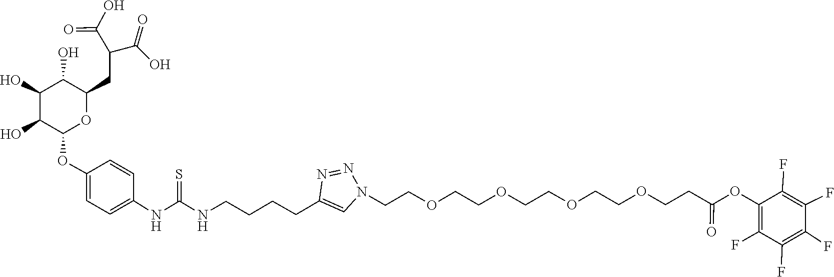 [SMILES: O=C(CCOCCOCCOCCOCCn1cc(CCCCNC(=S)Nc2ccc(O[C@H]3O[C@H](CC(C(=O)O)C(=O)O)[C@@H](O)[C@H](O)[C@@H]3O)cc2)nn1)Oc1c(F)c(F)c(F)c(F)c1F] |
| 538 (I-64) | 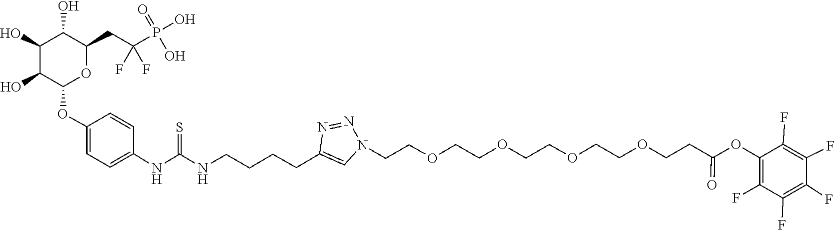 [SMILES: O=C(CCOCCOCCOCCOCCn1cc(CCCCNC(=S)Nc2ccc(O[C@H]3O[C@H](CC(F)(F)P(=O)(O)O)[C@@H](O)[C@H](O)[C@@H]3O)cc2)nn1)Oc1c(F)c(F)c(F)c(F)c1F] |
| 539 (I-65) | 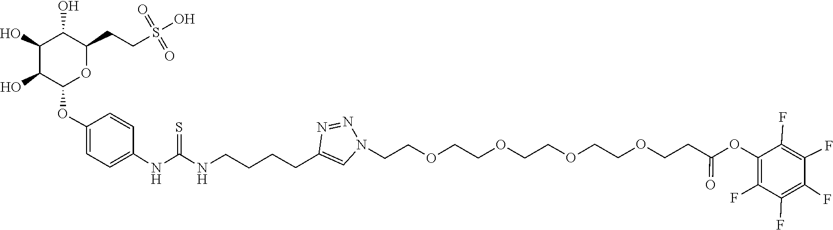 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F] |
| 541 (I-83) | 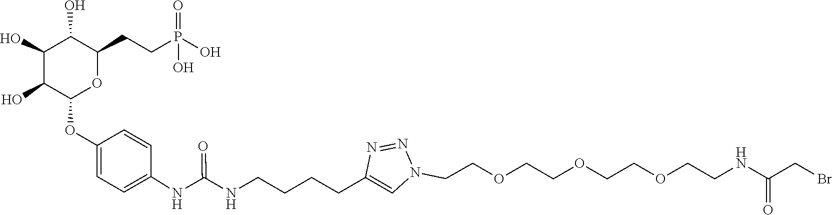 [SMILES: O=C(CBr)NCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1] |
| 542 (I-84) | 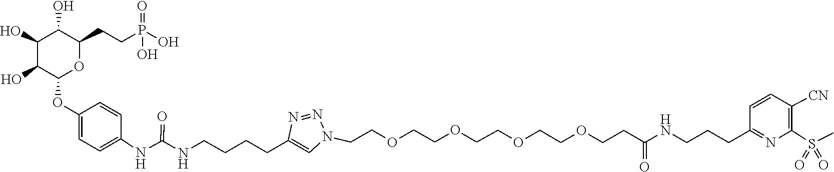 [SMILES: CS(=O)(=O)c1nc(CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)ccc1C#N] |
| 543 (I-85) | 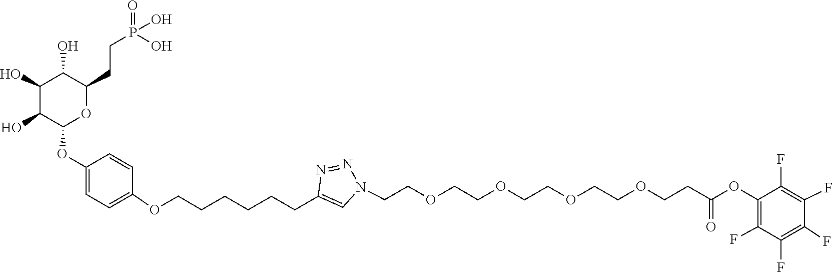 [SMILES: O=C(CCOCCOCCOCCOCCn1cc(CCCCCCOc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 544 (I-86) | 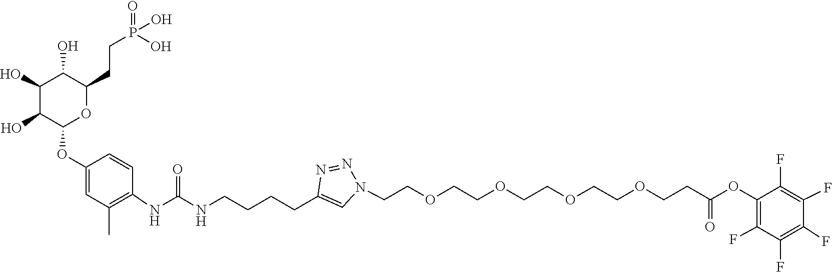 [SMILES: Cc1cc(O[C@H]2O[C@H](CCP(=O)(O)O)[C@@H](O)[C@H](O)[C@@H]2O)ccc1NC(=O)NCCCCc1cn(CCOCCOCCOCCOCCC(=O)Oc2c(F)c(F)c(F)c(F)c2F)nn1] |
| 545 (I-87) | 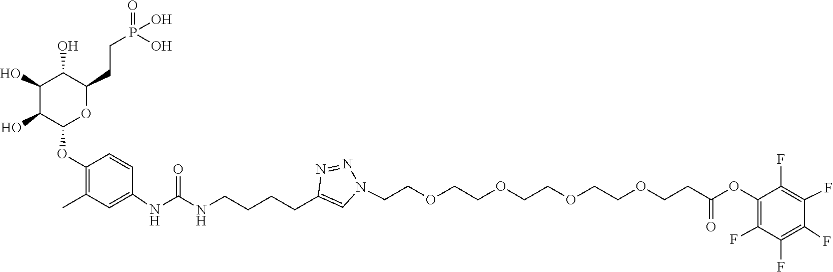 [SMILES: Cc1cc(NC(=O)NCCCCc2cn(CCOCCOCCOCCOCCC(=O)Oc3c(F)c(F)c(F)c(F)c3F)nn2)ccc1O[C@H]1O[C@H](CCP(=O)(O)O)[C@@H](O)[C@H](O)[C@@H]1O] |
| 546 (I-89) | 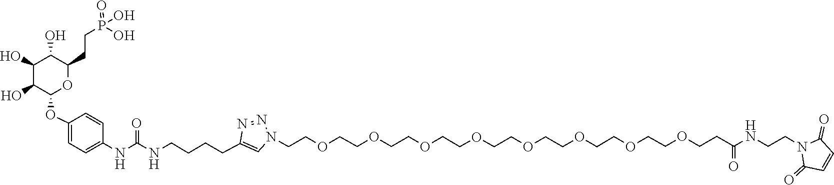 [SMILES: O=C(CCOCCOCCOCCOCCOCCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 547 (I-90) | 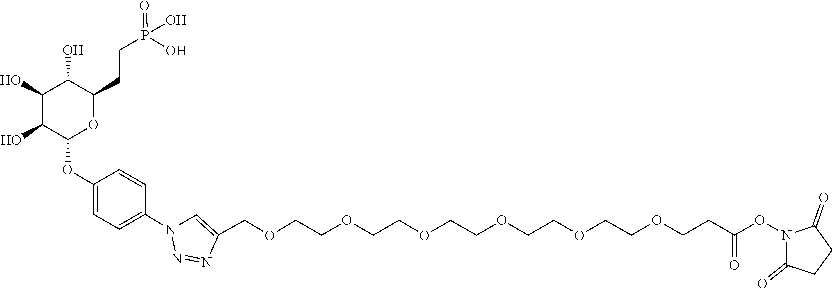 [SMILES: O=C(CCOCCOCCOCCOCCOCCOCc1cn(-c2ccc(O[C@H]3O[C@H](CCP(=O)(O)O)[C@@H](O)[C@H](O)[C@@H]3O)cc2)nn1)ON1C(=O)CCC1=O] |
| 548 (I-91) | 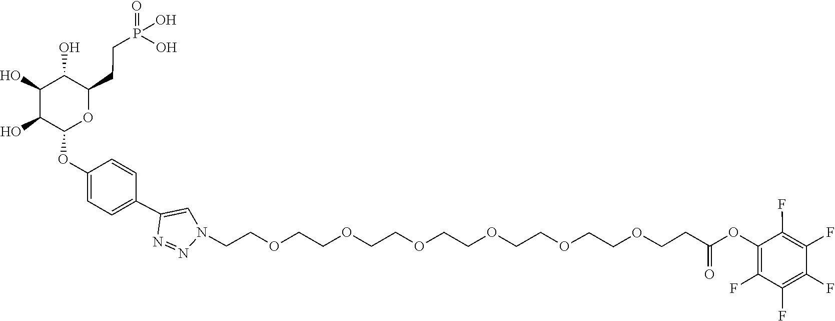 [SMILES: O=C(CCOCCOCCOCCOCCOCCOCCn1cc(-c2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 549 (I-92) | 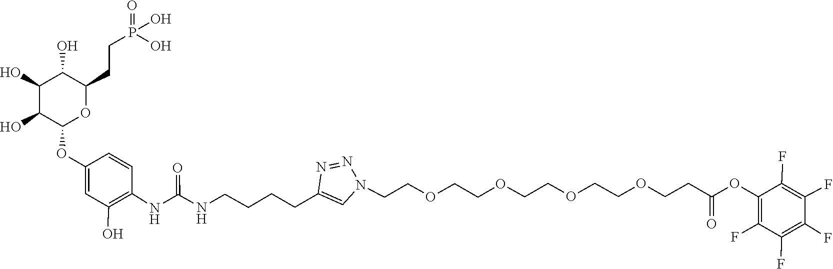 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1O] |
| 550 (I-93) | 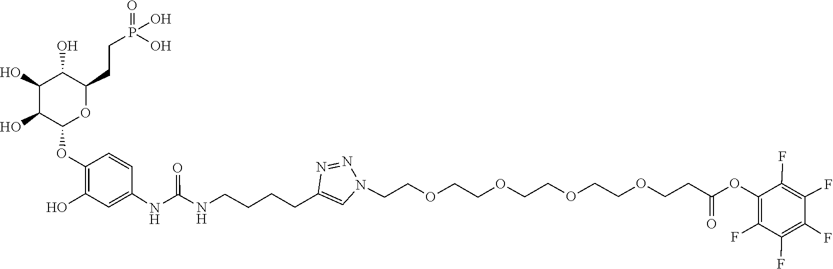 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)c(O)c1] |
| 551 (I-94) | 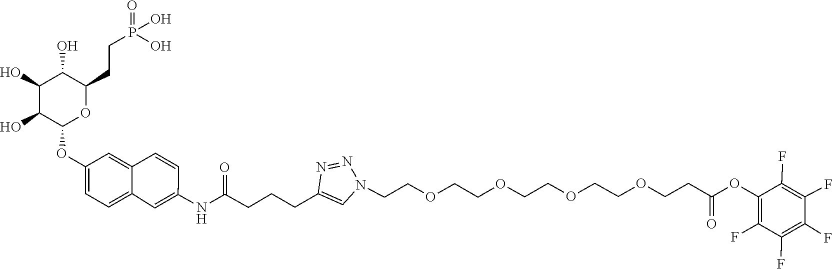 [SMILES: O=C(CCCc1cn(CCOCCOCCOCCOCCC(=O)Oc2c(F)c(F)c(F)c(F)c2F)nn1)Nc1ccc2cc(O[C@H]3O[C@H](CCP(=O)(O)O)[C@@H](O)[C@H](O)[C@@H]3O)ccc2c1] |
| 552 (I-95) | 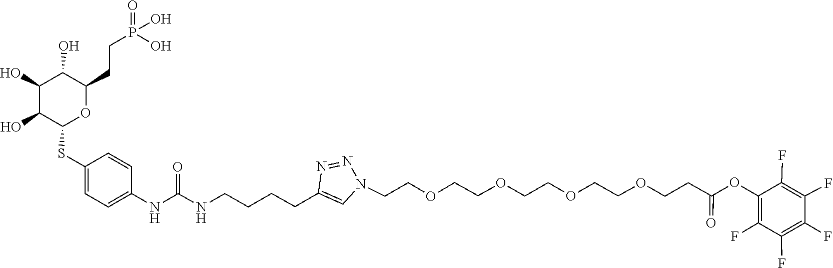 [SMILES: O=C(NCCCCc1cn(CCOCCOCCOCCOCCC(=O)Oc2c(F)c(F)c(F)c(F)c2F)nn1)Nc1ccc(S[C@H]2O[C@H](CCP(=O)(O)O)[C@@H](O)[C@H](O)[C@@H]2O)cc1] |
| 553 | 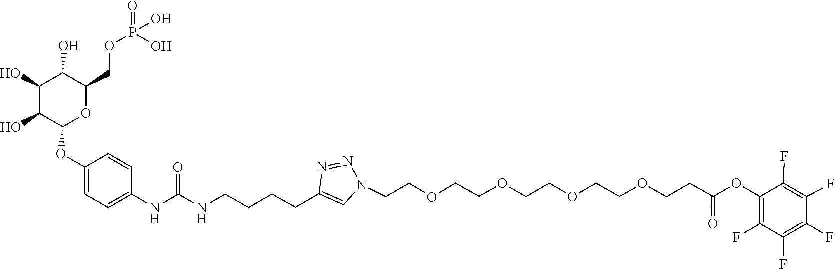 [SMILES: O=C(NCCCCc1cn(CCOCCOCCOCCOCCC(=O)Oc2c(F)c(F)c(F)c(F)c2F)nn1)Nc1ccc(O[C@H]2O[C@H](COP(=O)(O)O)[C@@H](O)[C@H](O)[C@@H]2O)cc1] |
| 554 (I-101B) | 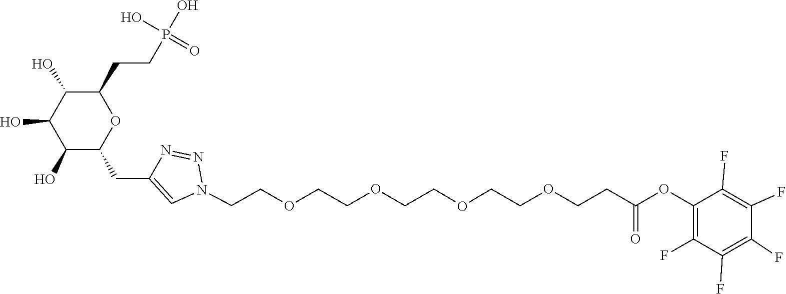 [SMILES: O=C(CCOCCOCCOCCOCCn1cc(C[C@H]2O[C@H](CCP(=O)(O)O)[C@@H](O)[C@H](O)[C@@H]2O)nn1)Oc1c(F)c(F)c(F)c(F)c1F] |
| 555 | 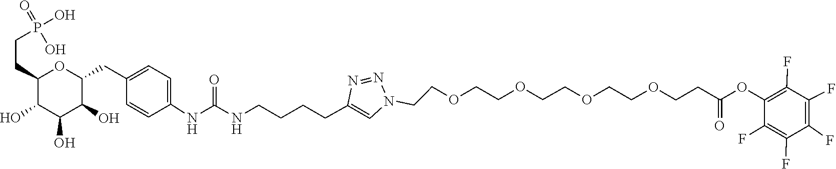 [SMILES: O=C(NCCCCc1cn(CCOCCOCCOCCOCCC(=O)Oc2c(F)c(F)c(F)c(F)c2F)nn1)Nc1ccc(C[C@H]2O[C@H](CCP(=O)(O)O)[C@@H](O)[C@H](O)[C@@H]2O)cc1] |
| 556 (I-104) | 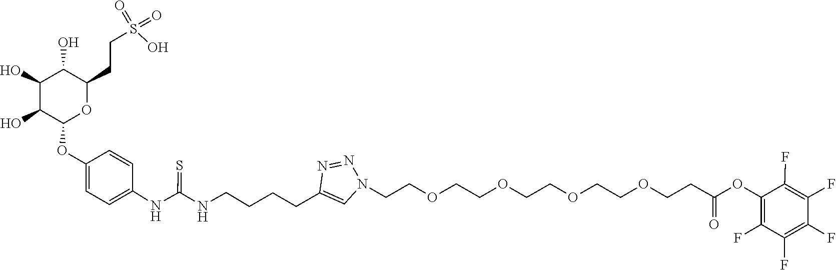 [SMILES: O=C(CCOCCOCCOCCOCCn1cc(CCCCNC(=S)Nc2ccc(O[C@H]3O[C@H](CCS(=O)(=O)O)[C@@H](O)[C@H](O)[C@@H]3O)cc2)nn1)Oc1c(F)c(F)c(F)c(F)c1F] |
| 557 (I-103) | 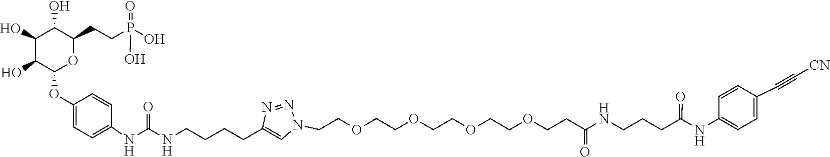 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1] |

TABLE 6
Exemplary M6PR binding compounds of formula (Ia)

| # | Compound Structure |
|---|---|
| 601 (I- 8) | 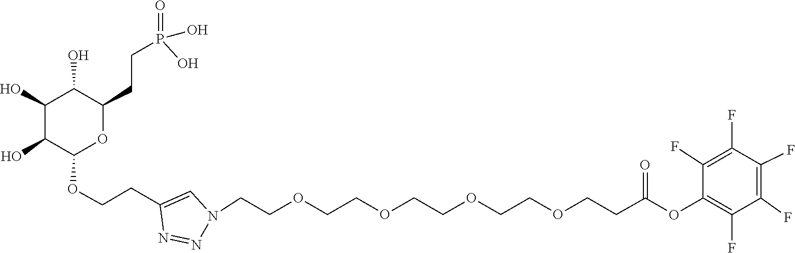 [SMILES: O=C(CCOCCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 602 (I- 9) | 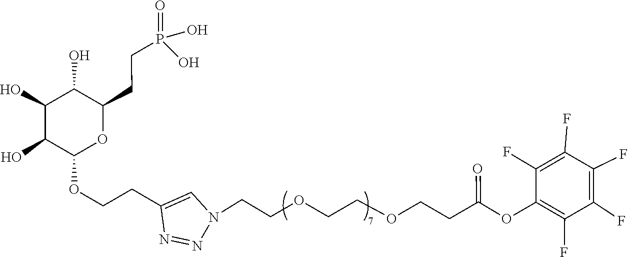 |
| 603 (I- 10) | 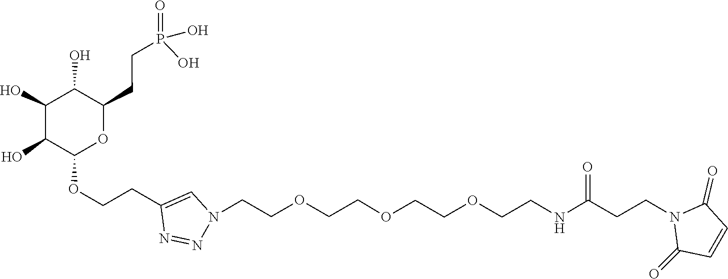 [SMILES: O=C(CCN1C(=O)C=CC1=O)NCCOCCOCCOCCn1cc(CCO[C@H]2O[C@H](CCP(=O)(O)O)[C@@H](O)[C@H](O)[C@@H]2O)nn1] |
| 604 (I- 11) | 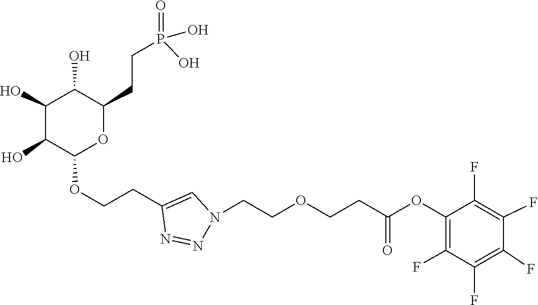 [SMILES: O=C(CCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 605 | 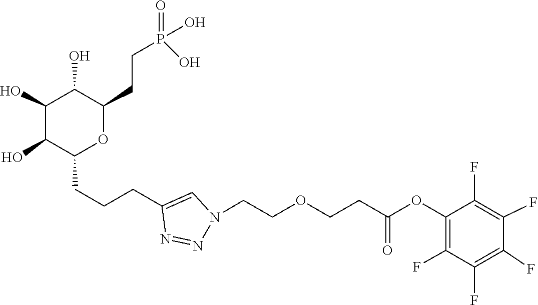 [SMILES: O=C(CCOCCn1cc(CCC[C@H]2O[C@H](CCP(=O)(O)O)[C@@H](O)[C@H](O)[C@@H]2O)nn1)Oc1c(F)c(F)c(F)c(F)c1F] |
| 606 (I- 13) | 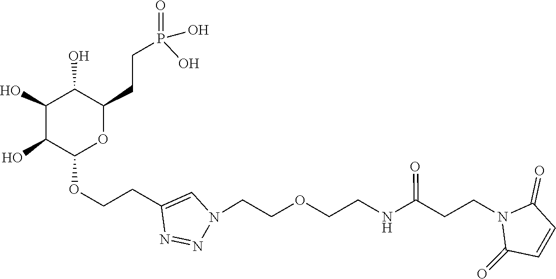 [SMILES: O=C(CCN1C(=O)C=CC1=O)NCCOCCn1cc(CCO[C@H]2O[C@H](CCP(=O)(O)O)[C@@H](O)[C@H](O)[C@@H]2O)nn1] |
| 607 (I- 14) | 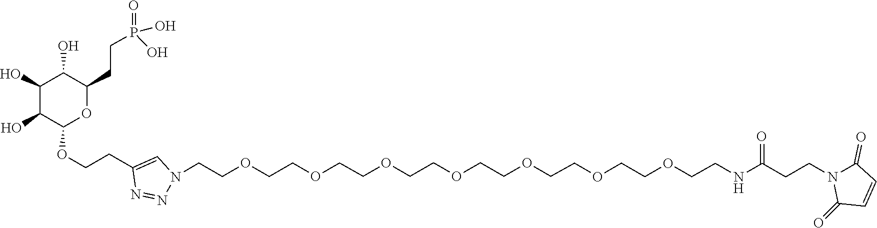 [SMILES: O=C(CCN1C(=O)C=CC1=O)NCCOCCOCCOCCOCCOCCOCCOCCn1cc(CCO[C@H]2O[C@H](CCP(=O)(O)O)[C@@H](O)[C@H](O)[C@@H]2O)nn1] |
| 608 (I- 15) | 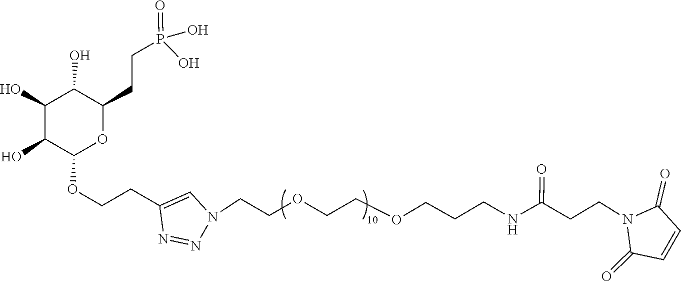 |
| 609 | 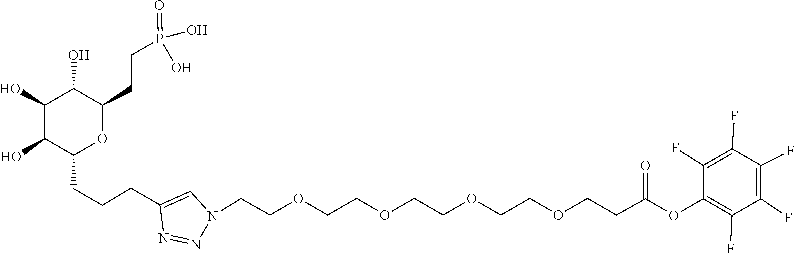 [SMILES: O=C(CCOCCOCCOCCOCCn1cc(CCC[C@H]2O[C@H](CCP(=O)(O)O)[C@@H](O)[C@H](O)[C@@H]2O)nn1)Oc1c(F)c(F)c(F)c(F)c1F] |
| 610 | 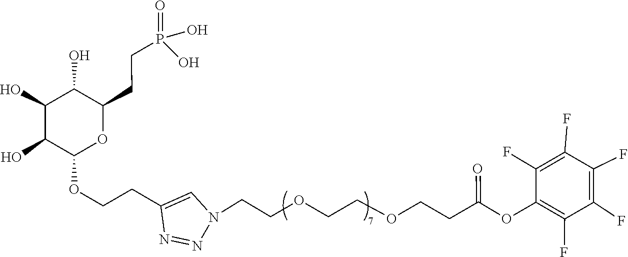 |
| 611 | 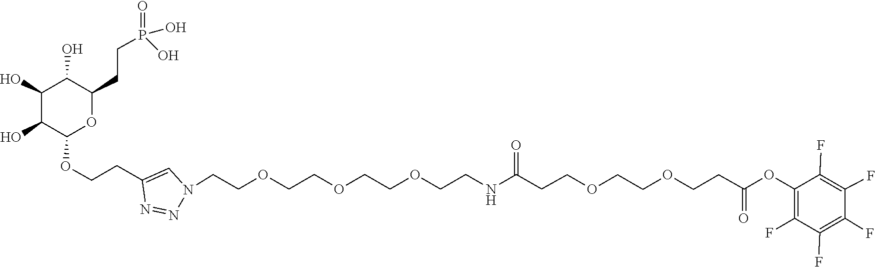 [SMILES: O=C(CCOCCOCCC(=O)Oc1c(F)c(F)c(F)c(F)c1F)NCCOCCOCCOCCn1cc(CCO[C@H]2O[C@H](CCP(=O)(O)O)[C@@H](O)[C@H](O)[C@@H]2O)nn1] |
| 612 (k = 4, I = 0) (I- 33) 613 (k = 0, I = 12) (I- 34) 614 (k = 2, I = 6) (I- 35) | 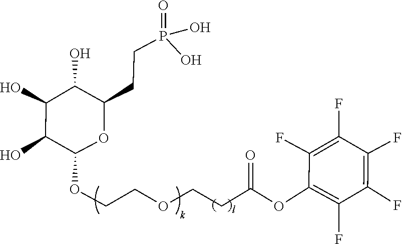 |
| 615 | 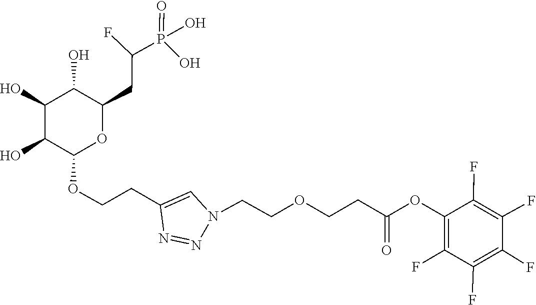 [SMILES: O=C(CCOCCn1cc(CCO[C@H]2O[C@H](CC(F)P(=O)(O)O)[C@@H](O)[C@H](O)[C@@H]2O)nn1)Oc1c(F)c(F)c(F)c(F)c1F] |
| 616 | 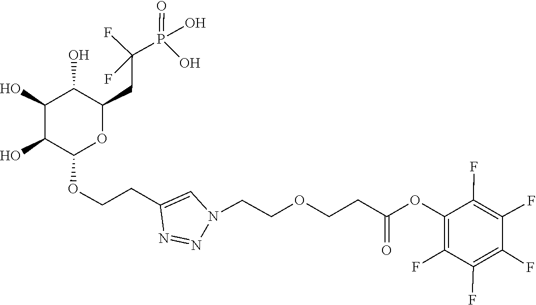 [SMILES: O=C(CCOCCn1cc(CCO[C@H]2O[C@H](CC(F)(F)P(=O)(O)O)[C@@H](O)[C@H](O)[C@@H]2O)nn1)Oc1c(F)c(F)c(F)c(F)c1F] |
| 617 | 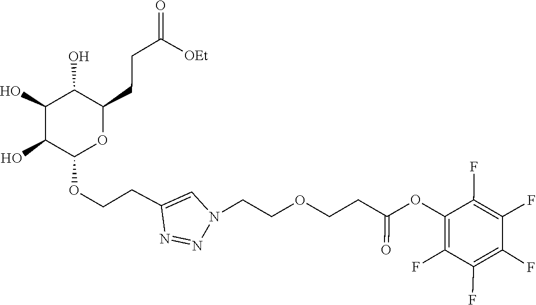 [SMILES: CCOC(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 618 | 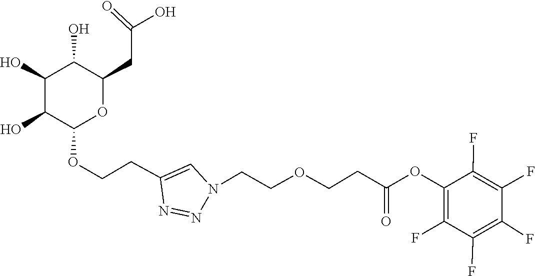 [SMILES: O=C(O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 619 | 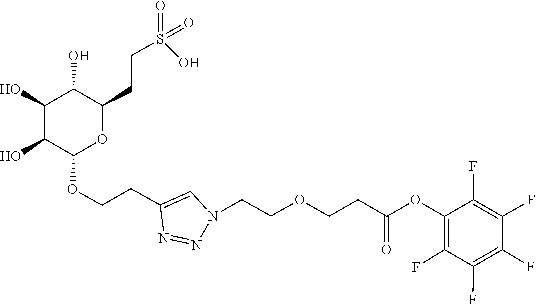 [SMILES: O=C(CCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 620 | 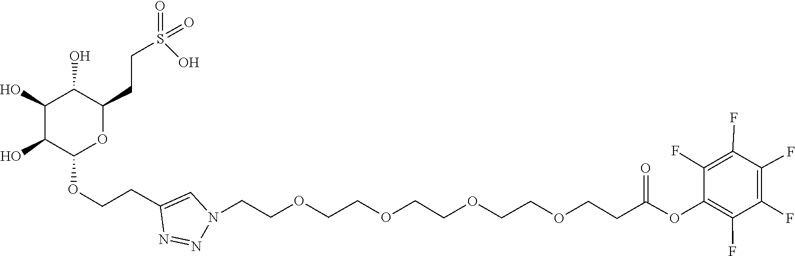 [SMILES: O=C(CCOCCOCCOCCOCCn1cc(CCO[C@H]2O[C@H](CCS(=O)(=O)O)[C@@H](O)[C@H](O)[C@@H]2O)nn1)Oc1c(F)c(F)c(F)c(F)c1F] |
| 621 | 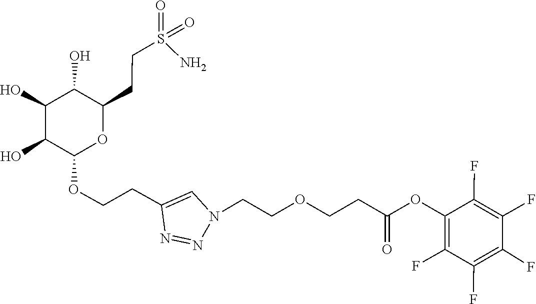 [SMILES: NS(=O)(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 622 | 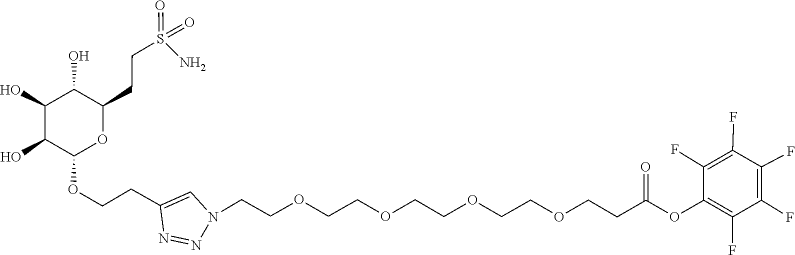 [SMILES: NS(=O)(=O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 623 | 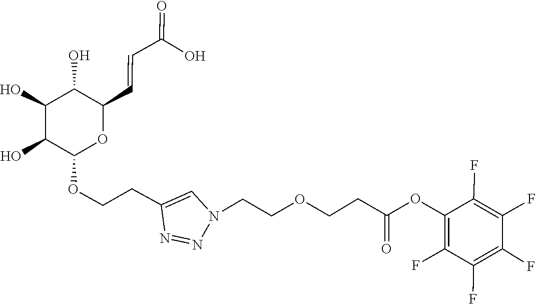 [SMILES: O=C(O)/C=C/[C@H]1O[C@H](OCCc2cn(CCOCCC(=O)Oc3c(F)c(F)c(F)c(F)c3F)nn2)[C@@H](O)[C@@H](O)[C@@H]1O] |
| 624 | 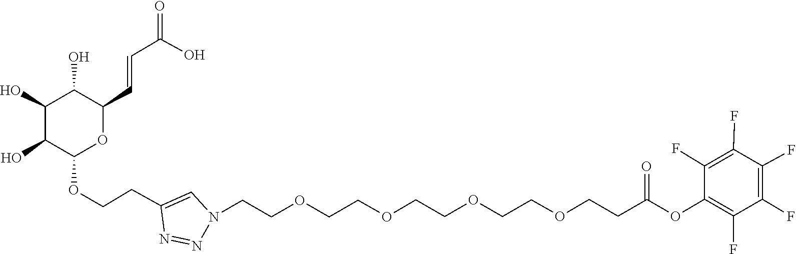 [SMILES: O=C(O)/C=C/[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 625 | 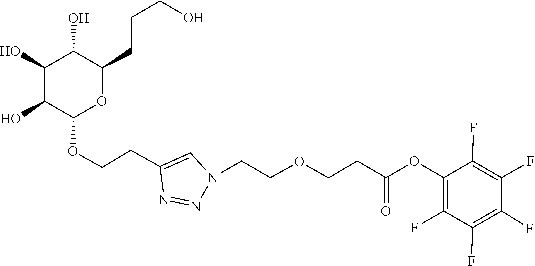 [SMILES: O=C(CCOCCn1cc(CCO[C@H]2O[C@H](CCCO)[C@@H](O)[C@H](O)[C@@H]2O)nn1)Oc1c(F)c(F)c(F)c(F)c1F] |
| 626 | 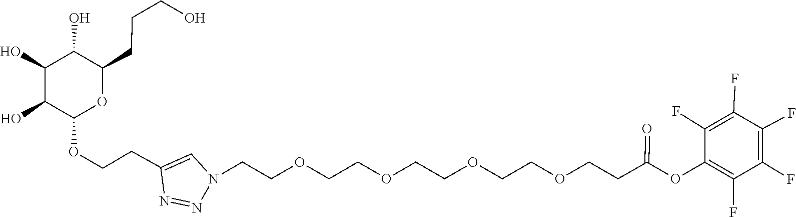 [SMILES: O=C(CCOCCOCCOCCOCCn1cc(CCO[C@H]2O[C@H](CCCO)[C@@H](O)[C@H](O)[C@@H]2O)nn1)Oc1c(F)c(F)c(F)c(F)c1F] |
| 627 | 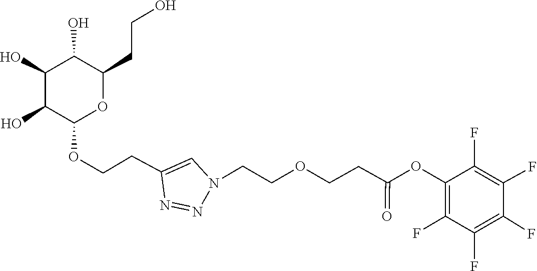 [SMILES: O=C(CCOCCn1cc(CCO[C@H]2O[C@H](CCO)[C@@H](O)[C@H](O)[C@@H]2O)nn1)Oc1c(F)c(F)c(F)c(F)c1F] |
| 628 | 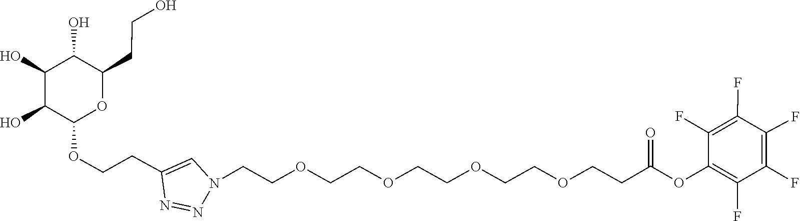 [SMILES: O=C(CCOCCOCCOCCOCCn1cc(CCO[C@H]2O[C@H](CCO)[C@@H](O)[C@H](O)[C@@H]2O)nn1)Oc1c(F)c(F)c(F)c(F)c1F] |
| 629 | 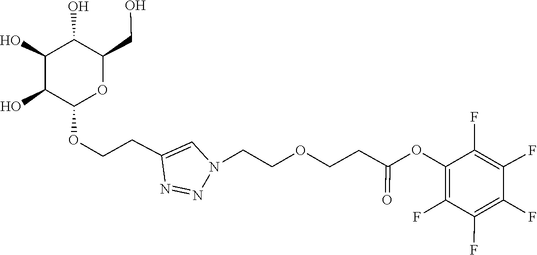 [SMILES: O=C(CCOCCn1cc(CCO[C@H]2O[C@H](CO)[C@@H](O)[C@H](O)[C@@H]2O)nn1)Oc1c(F)c(F)c(F)c(F)c1F] |
| 630 | 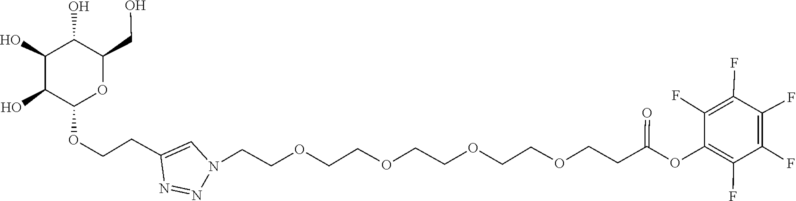 [SMILES: O=C(CCOCCOCCOCCOCCn1cc(CCO[C@H]2O[C@H](CO)[C@@H](O)[C@H](O)[C@@H]2O)nn1)Oc1c(F)c(F)c(F)c(F)c1F] |
| 631 | 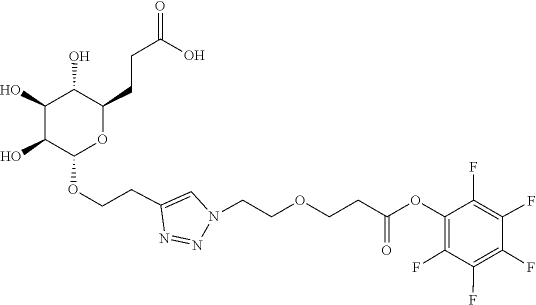 [SMILES: O=C(O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 632 | 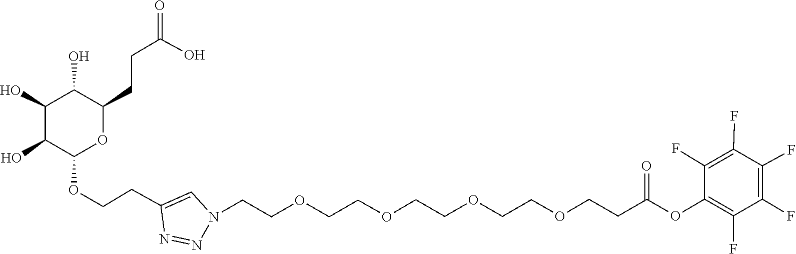 [SMILES: O=C(O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 633 | 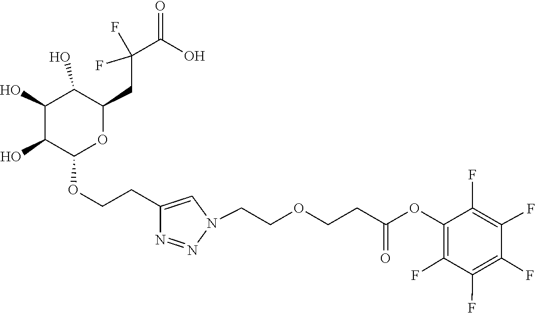 [SMILES: O=C(CCOCCn1cc(CCO[C@H]2O[C@H](CC(F)(F)C(=O)O)[C@@H](O)[C@H](O)[C@@H]2O)nn1)Oc1c(F)c(F)c(F)c(F)c1F] |
| 634 | 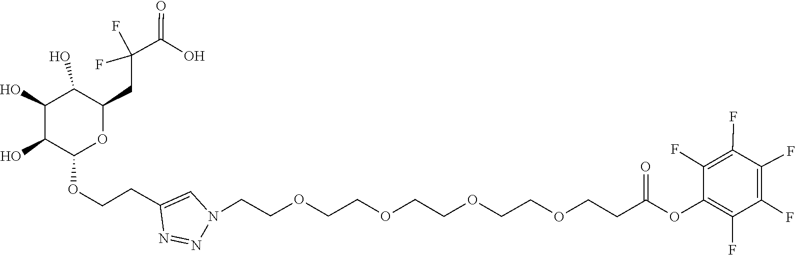 [SMILES: O=C(CCOCCOCCOCCOCCn1cc(CCO[C@H]2O[C@H](CC(F)(F)C(=O)O)[C@@H](O)[C@H](O)[C@@H]2O)nn1)Oc1c(F)c(F)c(F)c(F)c1F] |
| 635 | 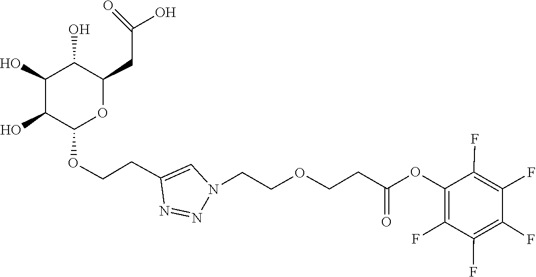 [SMILES: O=C(O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 636 | 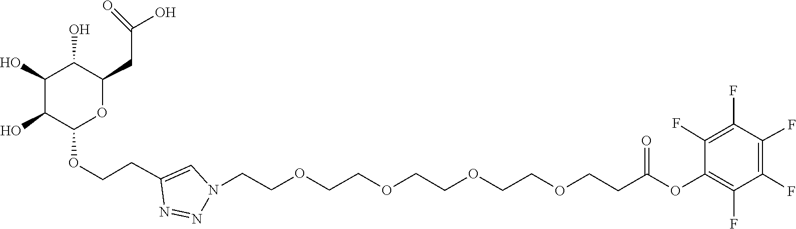 [SMILES: O=C(O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 637 | 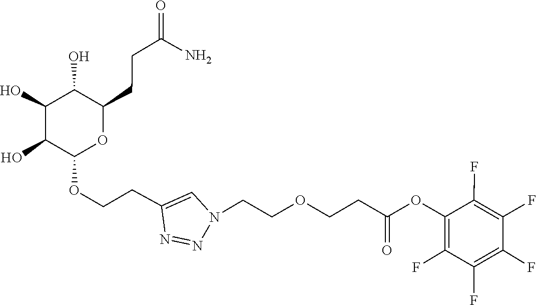 [SMILES: NC(=O)CC[C@H]1O[C@H](OCCc2cn(CCOCCC(=O)Oc3c(F)c(F)c(F)c(F)c3F)nn2)[C@@H](O)[C@@H](O)[C@@H]1O] |
| 638 | 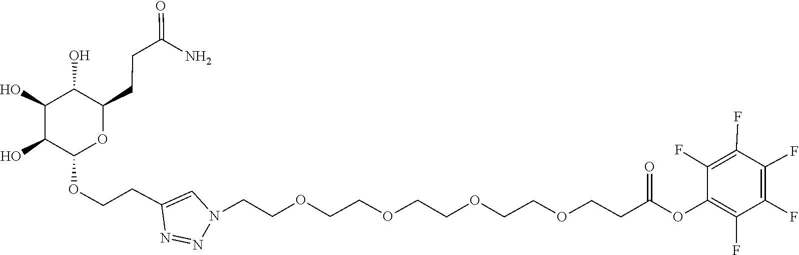 [SMILES: NC(=O)CC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 639 | 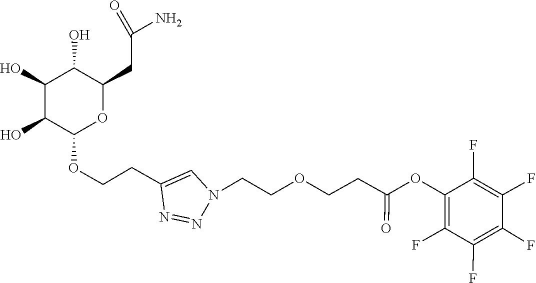 [SMILES: NC(=O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 640 | 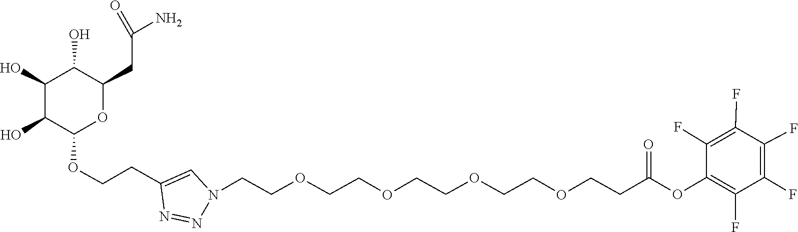 [SMILES: NC(=O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 641 | 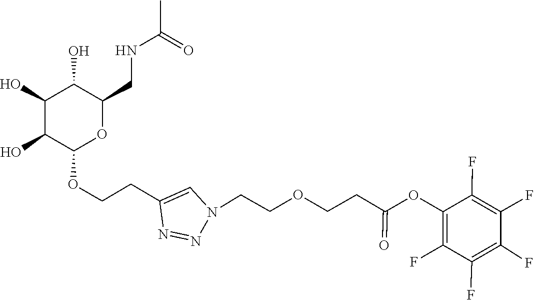 [SMILES: CC(=O)NC[C@H]1O[C@H](OCCc2cn(CCOCCC(=O)Oc3c(F)c(F)c(F)c(F)c3F)nn2)[C@@H](O)[C@@H](O)[C@@H]1O] |
| 642 | 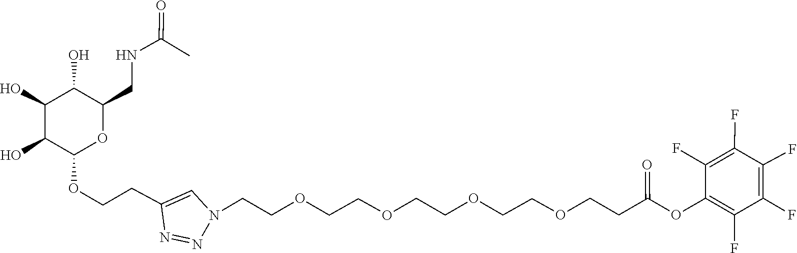 [SMILES: CC(=O)NC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 643 | 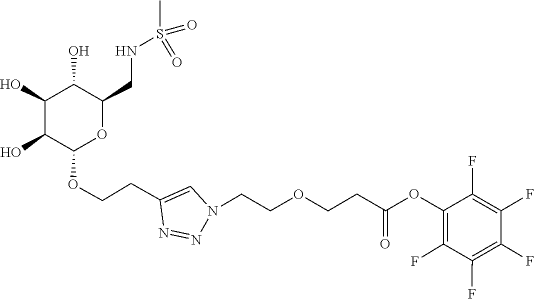 [SMILES: CS(=O)(=O)NC[C@H]1O[C@H](OCCc2cn(CCOCCC(=O)Oc3c(F)c(F)c(F)c(F)c3F)nn2)[C@@H](O)[C@@H](O)[C@@H]1O] |
| 644 | 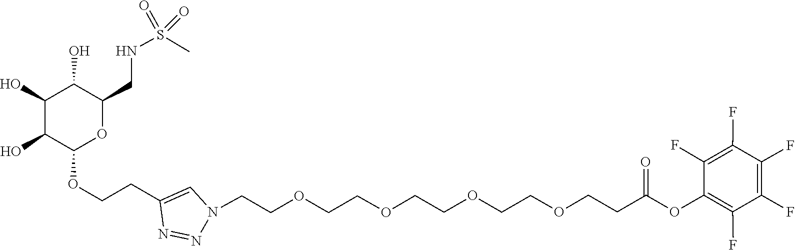 [SMILES: CS(=O)(=O)NC[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 645 | 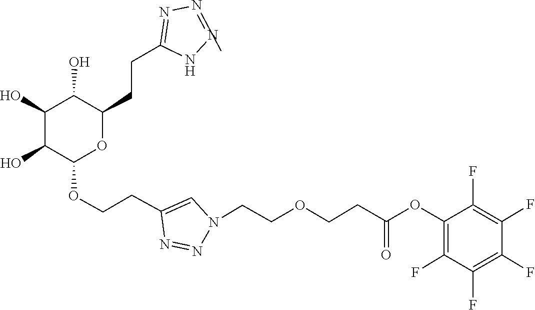 [SMILES: O=C(CCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 646 | 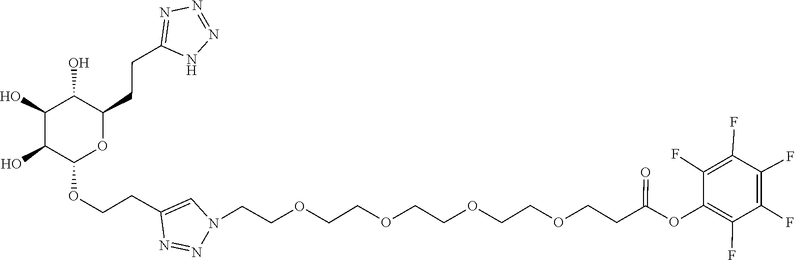 [SMILES: O=C(CCOCCOCCOCCOCCn1cc(CCO[C@H]2O[C@H](CCc3nnn[nH]3)[C@@H](O)[C@H](O)[C@@H]2O)nn1)Oc1c(F)c(F)c(F)c(F)c1F] |
| 647 | 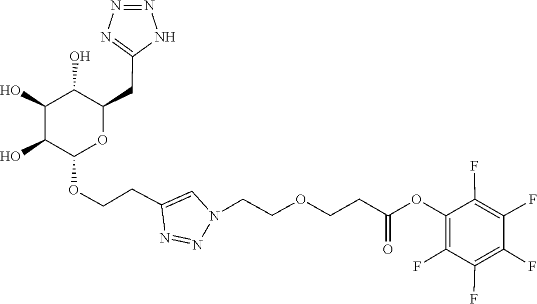 [SMILES: O=C(CCOCCn1cc(CCO[C@H]2O[C@H](Cc3nnn[nH]3)[C@@H](O)[C@H](O)[C@@H]2O)nn1)Oc1c(F)c(F)c(F)c(F)c1F] |
| 648 | 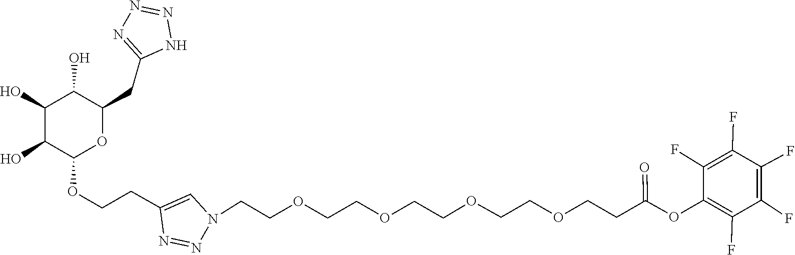 [SMILES: O=C(CCOCCOCCOCCOCCn1cc(CCO[C@H]2O[C@H](Cc3nnn[nH]3)[C@@H](O)[C@H](O)[C@@H]2O)nn1)Oc1c(F)c(F)c(F)c(F)c1F] |
| 649 | 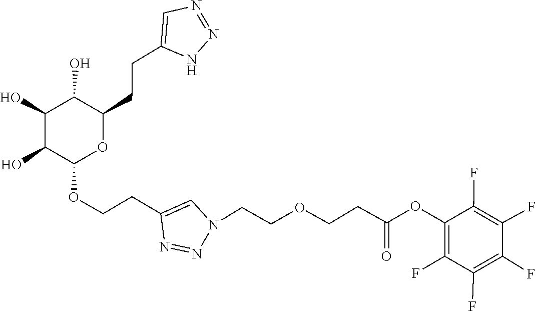 [SMILES: O=C(CCOCCn1cc(CCO[C@H]2O[C@H](CCc3cnn[nH]3)[C@@H](O)[C@H](O)[C@@H]2O)nn1)Oc1c(F)c(F)c(F)c(F)c1F] |
| 650 | 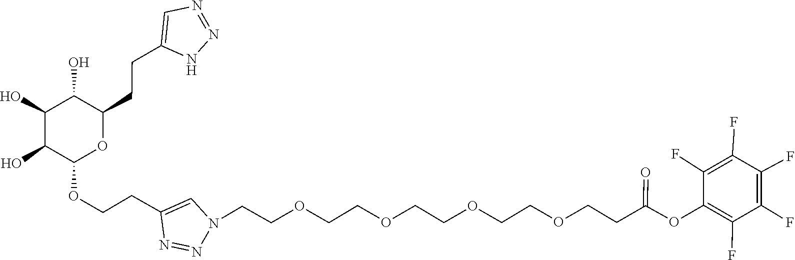 [SMILES: O=C(CCOCCOCCOCCOCCn1cc(CCO[C@H]2O[C@H](CCc3cnn[nH]3)[C@@H](O)[C@H](O)[C@@H]2O)nn1)Oc1c(F)c(F)c(F)c(F)c1F] |
| 651 | 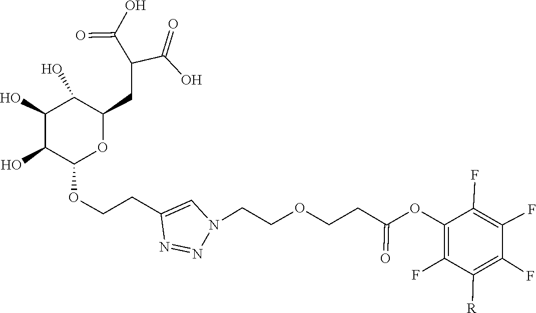 |
| 652 | 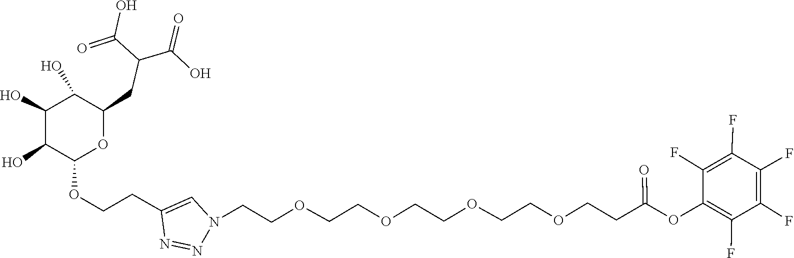 [SMILES: O=C(CCOCCOCCOCCOCCn1cc(CCO[C@H]2O[C@H](CC(C(=O)O)C(=O)O)[C@@H](O)[C@H](O)[C@@H]2O)nn1)Oc1c(F)c(F)c(F)c(F)c1F] |
| 653 | 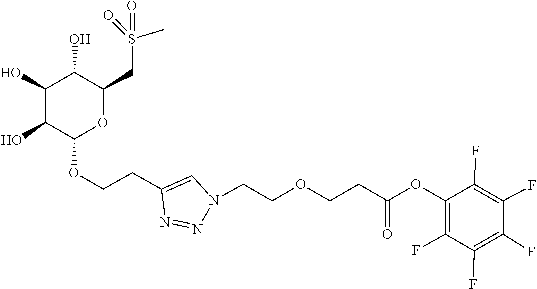 [SMILES: CS(=O)(=O)C[C@H]1O[C@H](OCCc2cn(CCOCCC(=O)Oc3c(F)c(F)c(F)c(F)c3F)nn2)[C@@H](O)[C@@H](O)[C@@H]1O] |
| 654 | 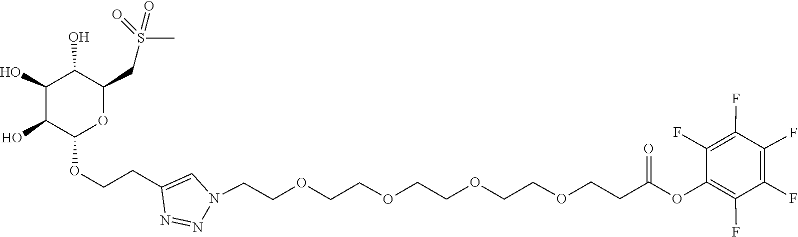 [SMILES: CS(=O)(=O)C[C@H]1O[C@H](OCCc2cn(CCOCCOCCOCCOCCC(=O)Oc3c(F)c(F)c(F)c(F)c3F)nn2)[C@@H](O)[C@@H](O)[C@@H]1O] |
| 656 (I- 37) | 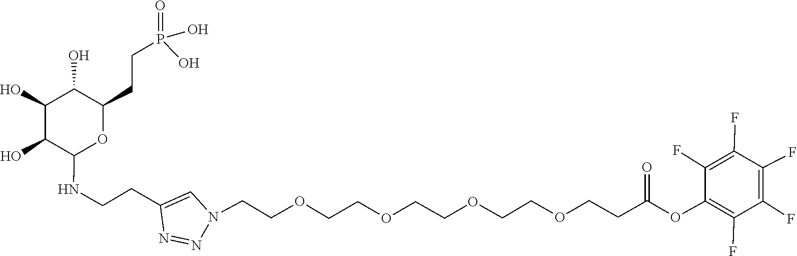 [SMILES: O=C(CCOCCOCCOCCOCCn1cc(CCNC2O[C@H](CCP(=O)(O)O)[C@@H](O)[C@H](O)[C@@H]2O)nn1)Oc1c(F)c(F)c(F)c(F)c1F] |

In certain embodiments of formula (Ia)-(Ib), n is 2. In certain embodiments of formula (Ia)-(Ib), n is 2, and Y is a chemoselective ligation group. In certain embodiments of formula (Ia)-(Ib), n is 3. In certain embodiments of formula (Ia)-(Ib), n is 3, and Y is a chemoselective ligation group.

Exemplary multivalent M6PR binding compounds are shown in Tables 7A-7B.

Exemplary multivalent ASGPR binding compounds are shown in Tables 8-9.

TABLE 7A
Multivalent M6PR binding compounds having chemoselective ligation group

| # | Structure |
|---|---|
| 701 (I-12) | 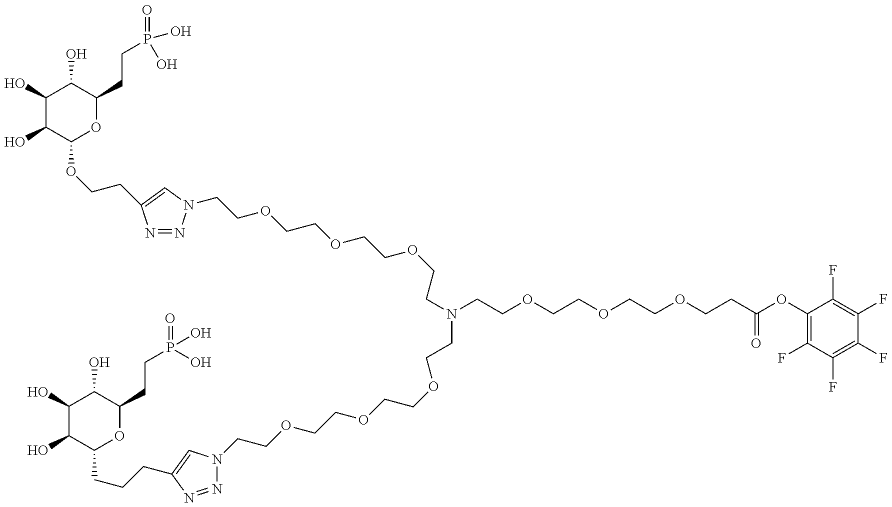 [SMILES: O=C(CCOCCOCCOCCN(CCOCCOCCOCCn1cc(CCC[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F] |
| 702 | 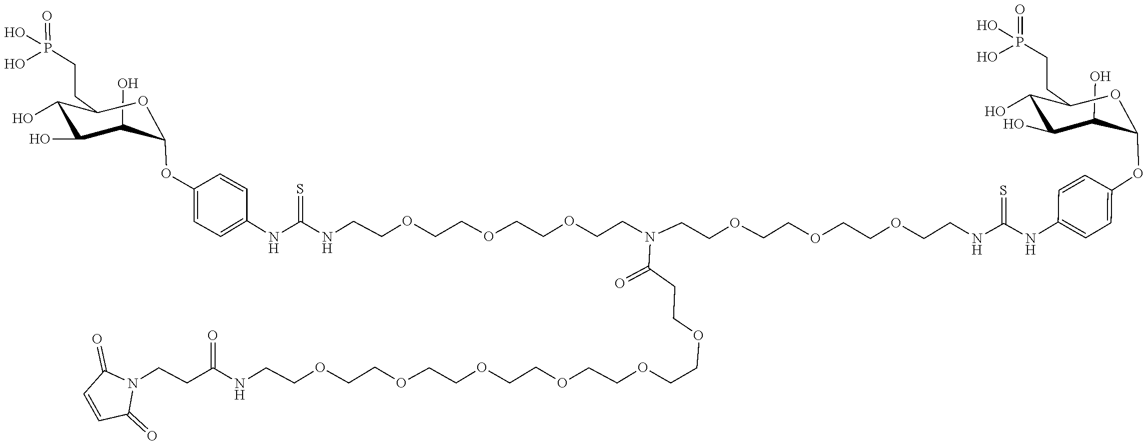 |
| 703 | 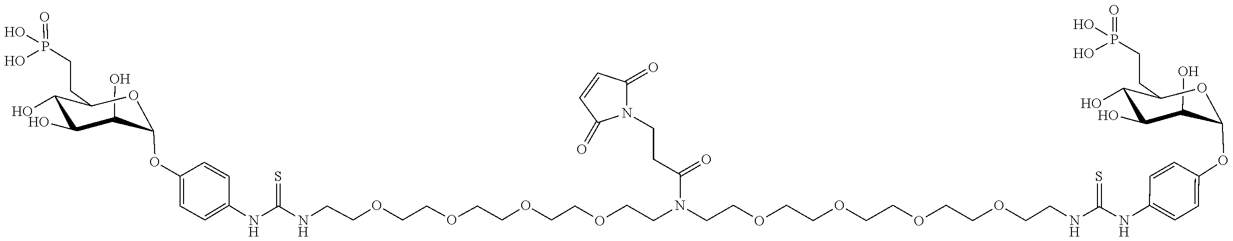 |
| 704 | 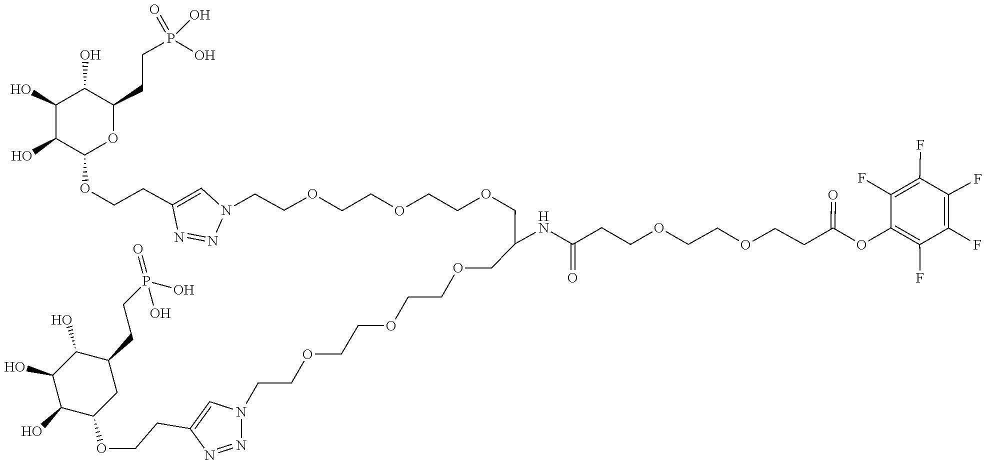 [SMILES: O=C(CCOCCOCCC(=O)Oc1c(F)c(F)c(F)c(F)c1F)NC(COCCOCCOCCn1cc(CCO[C@H]2O[C@H](CCP(=O)(O)O)[C@@H](O)[C@H](O)[C@@H]2O)nn1)COCCOCCOCCn1cc(CCO[C@H]2C[C@H](CCP(=O)(O)O)[C@@H](O)[C@H](O)[C@@H]2O)nn1] |
| 705 (I-40) | 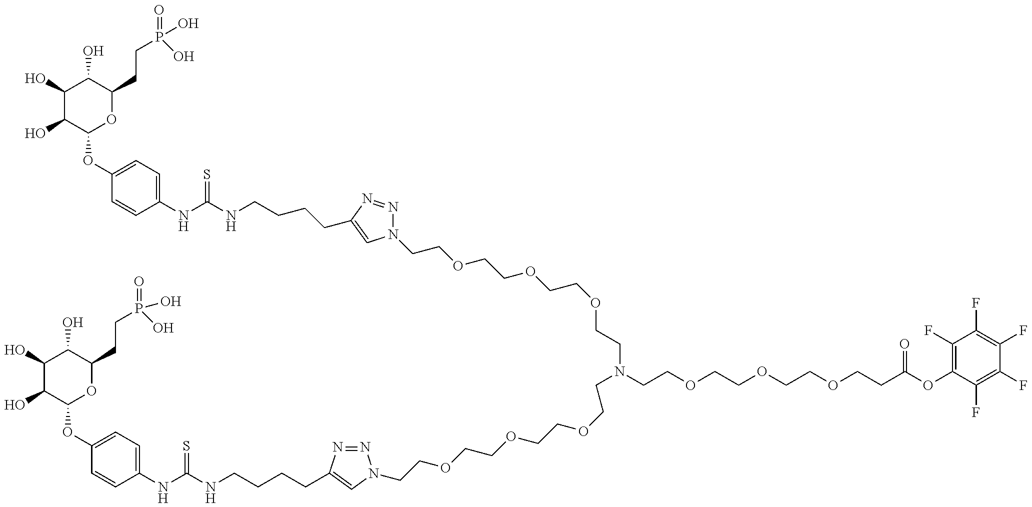 [SMILES: O=C(CCOCCOCCOCCN(CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)Oc1c(F)c(F)c(F)c(F)c1F] |
| 706 (I-41) | 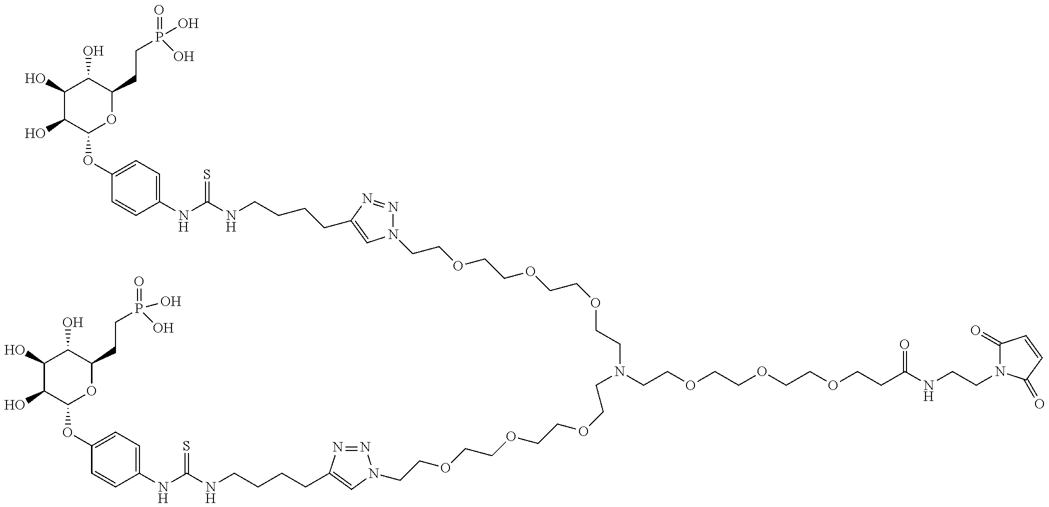 [SMILES: O=C(CCOCCOCCOCCN(CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 707 (I-43) | 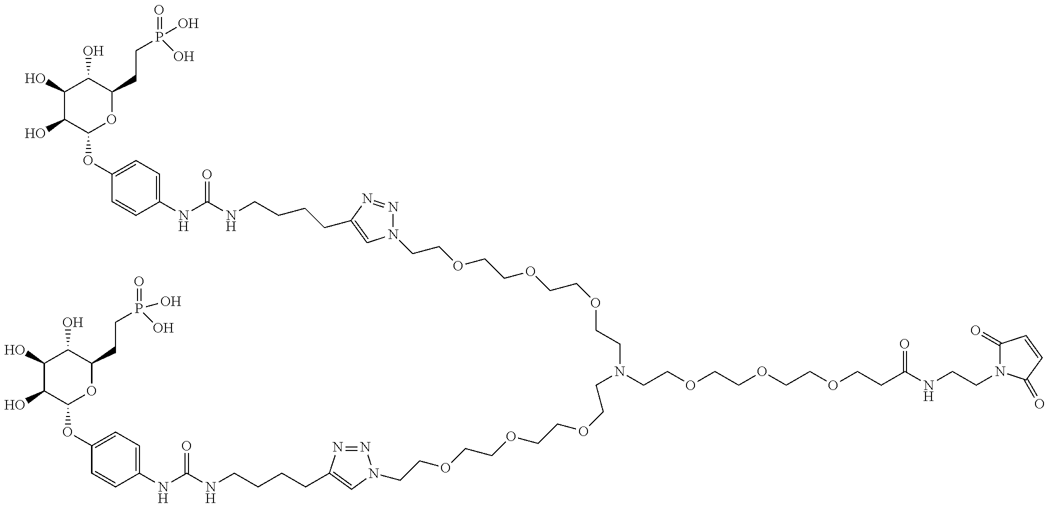 [SMILES: O=C(CCOCCOCCOCCN(CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 708 (I-58) | 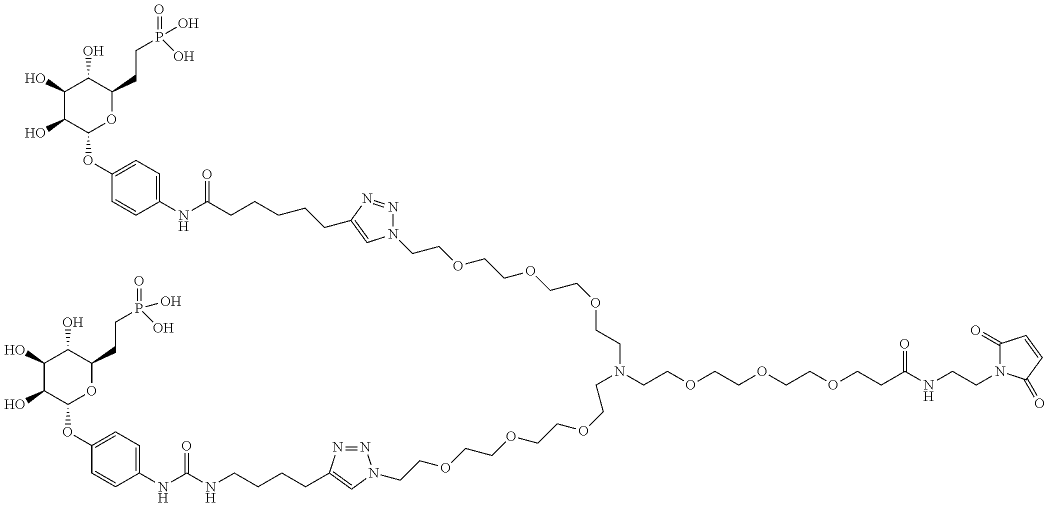 [SMILES: O=C(CCOCCOCCOCCN(CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |
| 709 (I-42) | 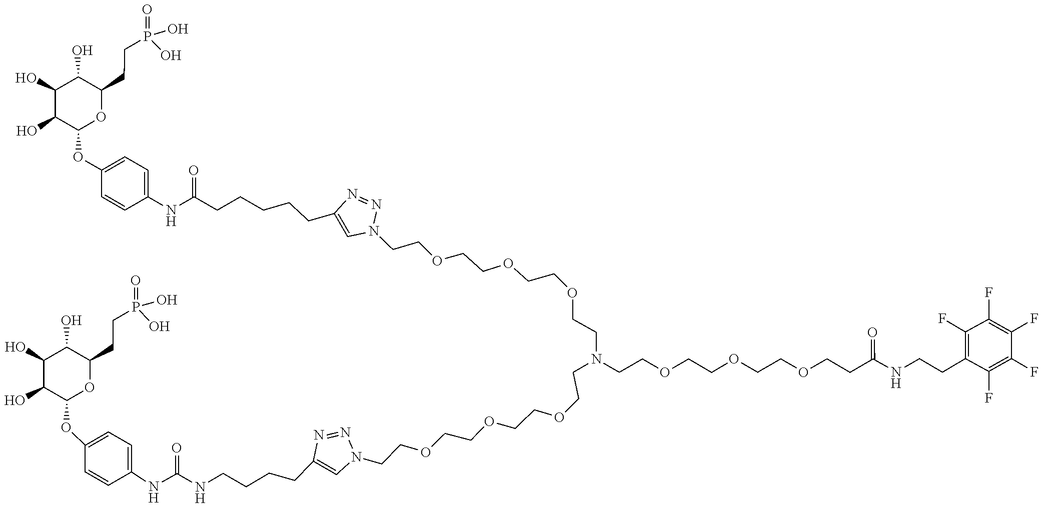 [SMILES: O=C(CCOCCOCCOCCN(CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCc1c(F)c(F)c(F)c(F)c1F] |
| 710 (I-53) | 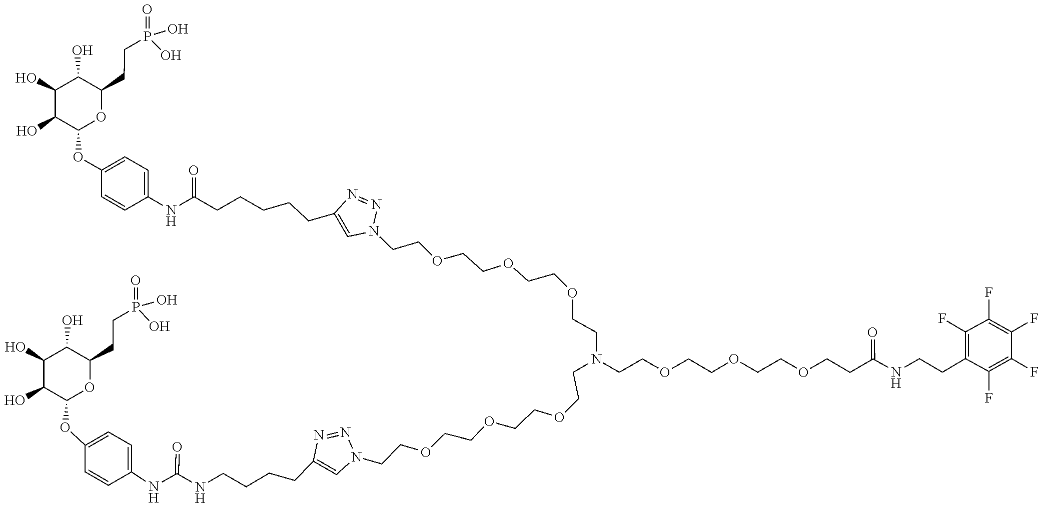 [SMILES: O=C(CCOCCOCCOCCN(CCOCCOCCOCCn1cc(CCCCCC(=O)Nc2ccc(O[C@H]3O[C@H](CCP(=O)(O)O)[C@@H](O)[C@H](O)[C@@H]3O)cc2)nn1)CCOCCOCCOCCn1cc(CCCCNC(=O)Nc2ccc(O[C@H]3O[C@H](CCP(=O)(O)O)[C@@H](O)[C@H](O)[C@@H]3O)cc2)nn1)NCCc1c(F)c(F)c(F)c(F)c1F] |
| 711 (I-96) | 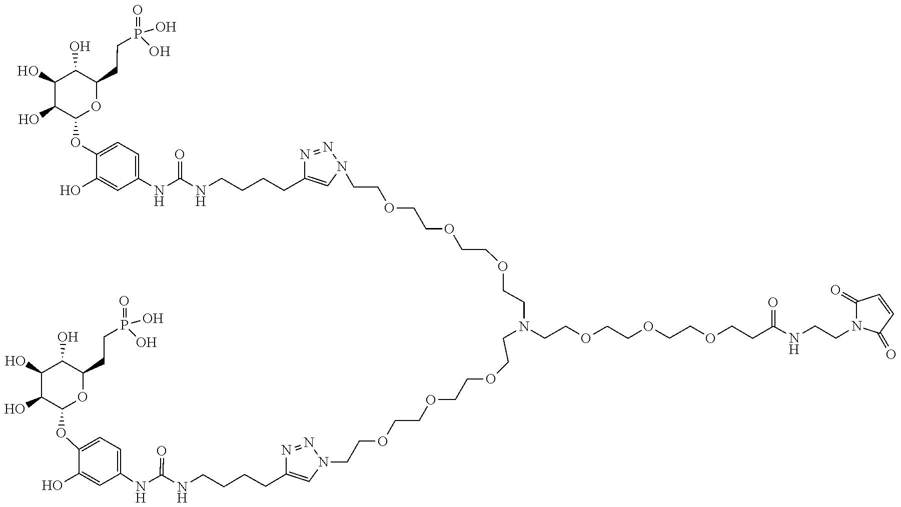 [SMILES: O=C(CCOCCOCCOCCN(CCOCCOCCOCCn1cc(CCCCNC(=O)Nc2ccc(O[C@H]3O[C@H](CCP(=O)(O)O)[C@@H](O)[C@H](O)[C@@H]3O)c(O)c2)nn1)CCOCCOCCOCCn1cc(CCCCNC(=O)Nc2ccc(O[C@H]3O[C@H](CCP(=O)(O)O)[C@@H](O)[C@H](O)[C@@H]3O)c(O)c2)nn1)NCCN1C(=O)C=CC1=O] |
| 712 | 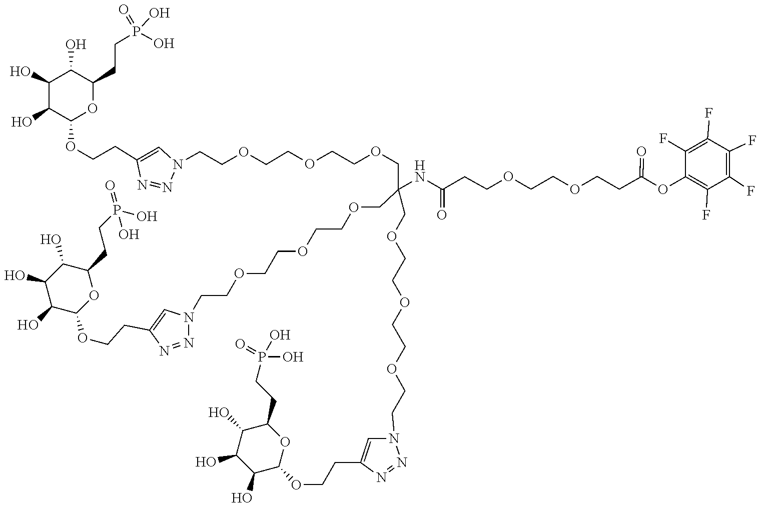 [SMILES: O=C(CCOCCOCCC(=O)Oc1c(F)c(F)c(F)c(F)c1F)NC(COCCOCCOCCn1cc(CCO[C@H]2O[C@H](CCP(=O)(O)O)[C@@H](O)[C@H](O)[C@@H]2O)nn1)(COCCOCCOCCn1cc(CCO[C@H]2O[C@H](CCP(=O)(O)O)[C@@H](O)[C@H](O)[C@@H]2O)nn1)COCCOCCOCCn1cc(CCO[C@H]2O[C@H](CCP(=O)(O)O)[C@@H](O)[C@H](O)[C@@H]2O)nn1] |
| 713 (I-44) | 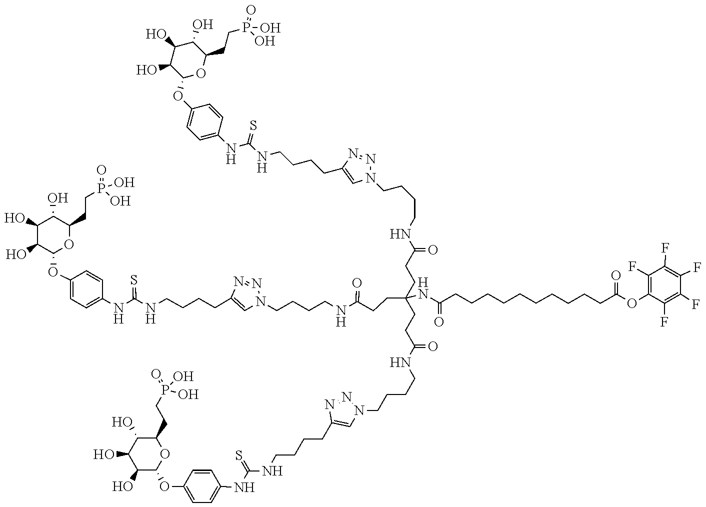 [SMILES: O=C(CCC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCCCCCCCCC(=O)Oc1c(F)c(F)c(F)c(F)c1F)NCCCCn1cc(CCCCNC(=S)Nc2ccc(O[C@H]3O[C@H](CCP(=O)(O)O)[C@@H](O)[C@H](O)[C@@H]3O)cc2)nn1] |
| 714 (I-45) | 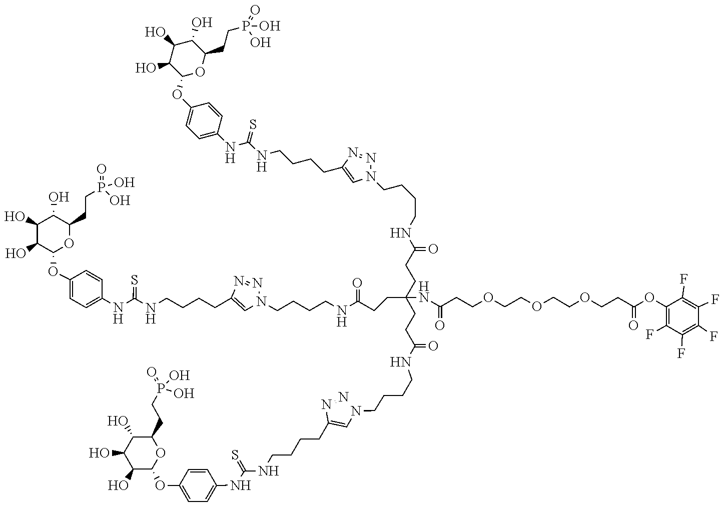 [SMILES: O=C(CCC(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCOCCOCCOCCC(=O)Oc1c(F)c(F)c(F)c(F)c1F)NCCCCn1cc(CCCCNC(=S)Nc2ccc(O[C@H]3O[C@H](CCP(=O)(O)O)[C@@H](O)[C@H](O)[C@@H]3O)cc2)nn1] |
| 715 (I-54) | 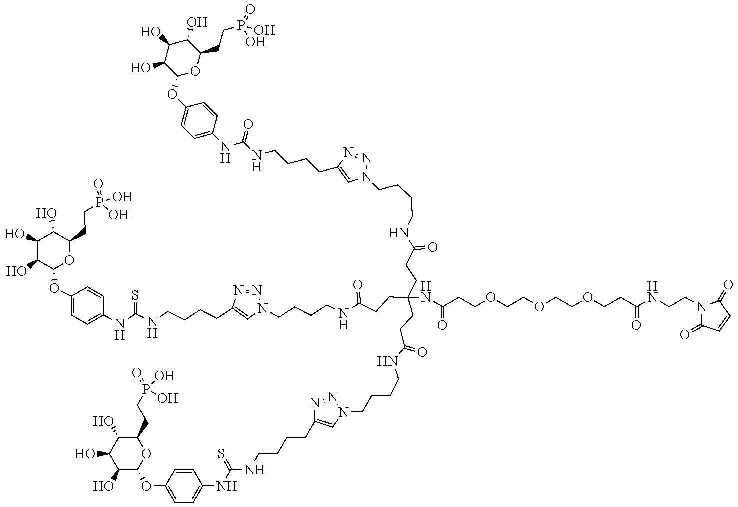 [SMILES: O=C(CCOCCOCCOCCC(=O)NC(CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)CCC(=O)NCCCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 716 | 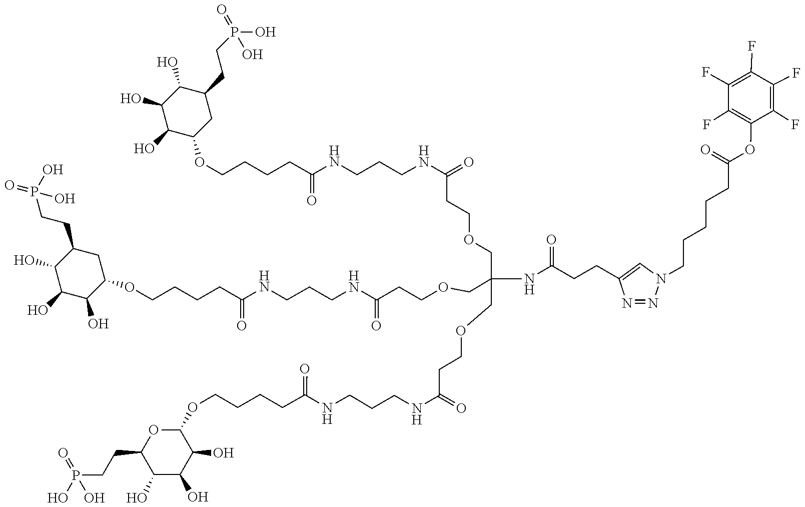 [SMILES: O=C(CCCCO[C@H]1O[C@H](CCP(=O)(O)O)[C@@H](O)[C@H](O)[C@@H]1O)NCCCNC(=O)CCOCC(COCCC(=O)NCCCNC(=O)CCCCO[C@H]1C[C@H](CCP(=O)(O)O)[C@@H](O)[C@H](O)[C@@H]1O)(COCCC(=O)NCCCNC(=O)CCCCO[C@H]1C[C@H](CCP(=O)(O)O)[C@@H](O)[C@H](O)[C@@H]1O)NC(=O)CCc1cn(CCCCCC(=O)Oc2c(F)c(F)c(F)c(F)c2F)nn1] |
| 717 (I-81) | 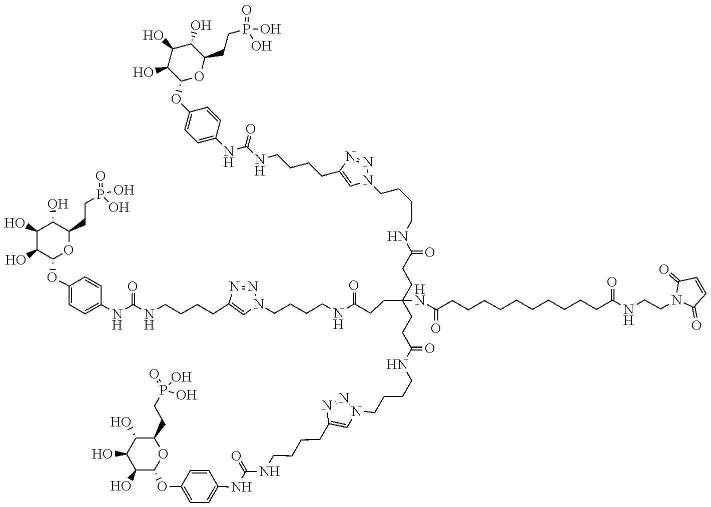 [SMILES: O=C(CCCCCCCCCCC(=O)NC(CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)(CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)CCC(=O)NCCCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |

In certain embodiments of formula (Ia)-(Ib), n is 2 or more (e.g., 3 or more, such as 3, 4, 5, or 6 or more) and the linker includes amino acid linking moieties that are branched and can be linked in a sequence together to provide for linkages via their sidechains (and optionally terminal groups) to multiple X ligands. In certain embodiments of formula (Ia), n is 3 or more, and Y is a chemoselective ligation group. In certain embodiments of formula (Ia), n is 4 or more, and Y is a chemoselective ligation group.

Exemplary multivalent compounds including amino acid residue linking moieties are shown in Table 7B.

TABLE 7B
Exemplary multivalent compounds including amino acid linking moieties

| # | Structure |
|---|---|
| 716 | 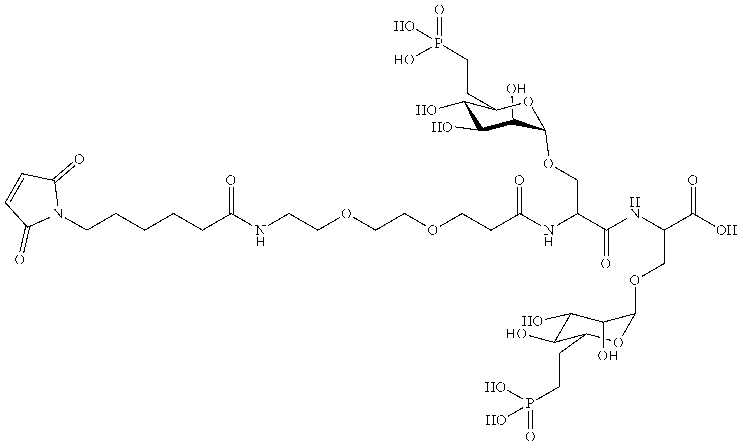 |
| 717 | 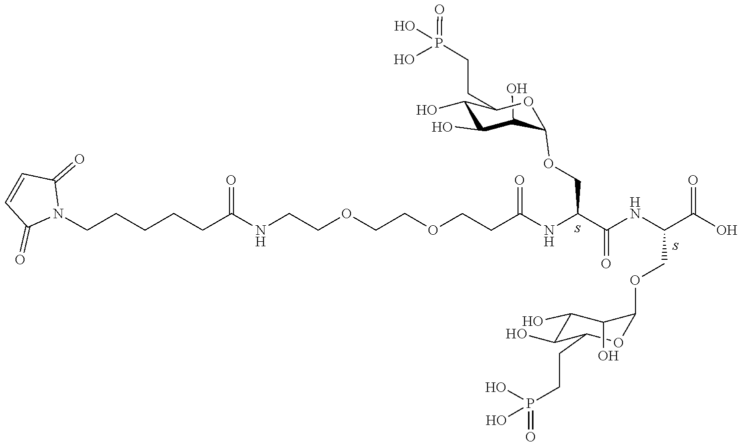 |
| 718 | 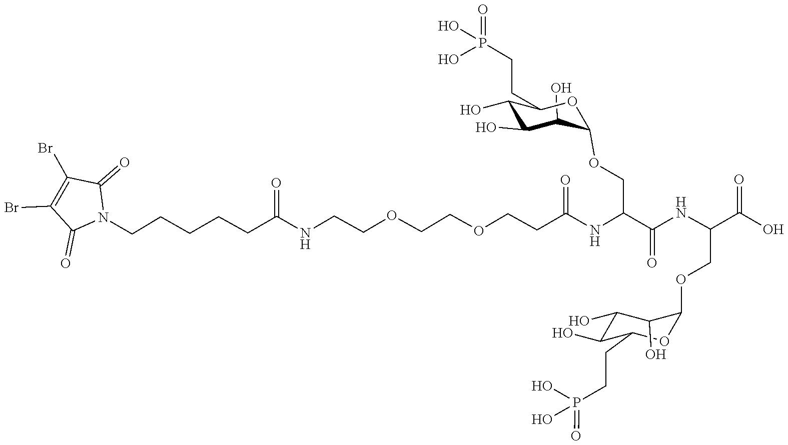 |
| 719 | 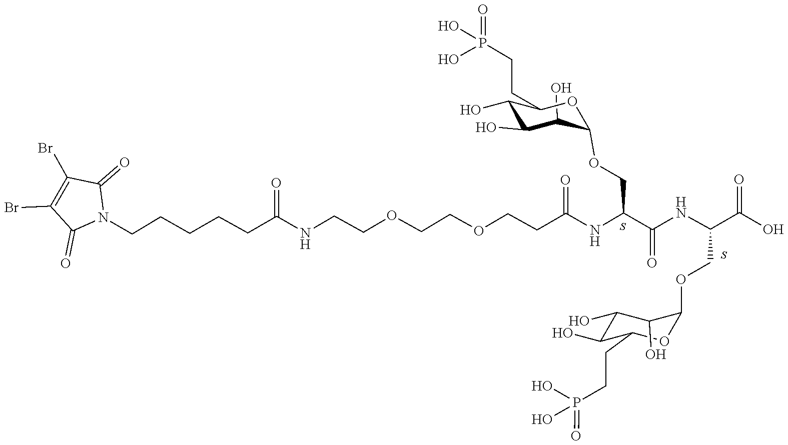 |
| 720 | 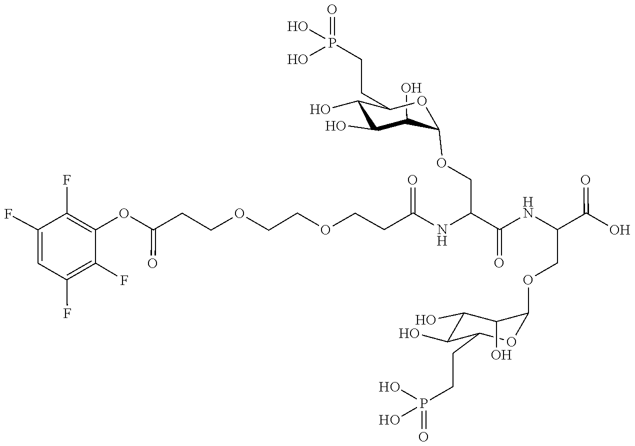 |
| 721 | 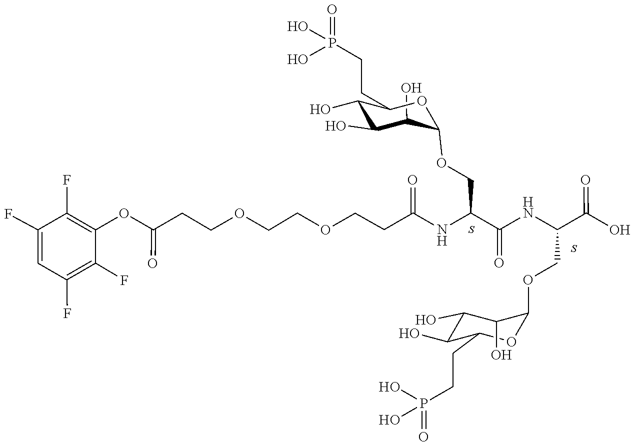 |
| 722 | 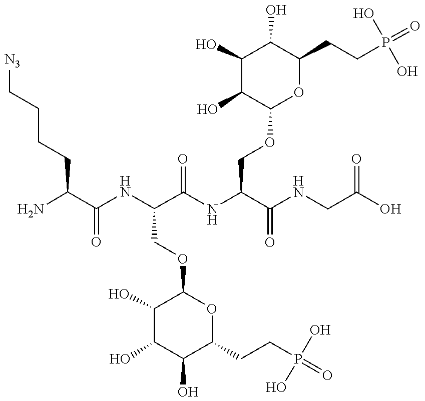 [SMILES: [N-]=[N+]=NCCCC[C@H](N)C(=O)N[C@@H](CO[C@H]1O[C@H](CCP(=O)(O)O)[C@@H](O)[C@H](O)[C@@H]1O)C(=O)N[C@@H](CO[C@H]1O[C@H](CCP(=O)(O)O)[C@@H](O)[C@H](O)[C@@H]1O)C(=O)NCC(=O)O] |
| 723 (I-97) | 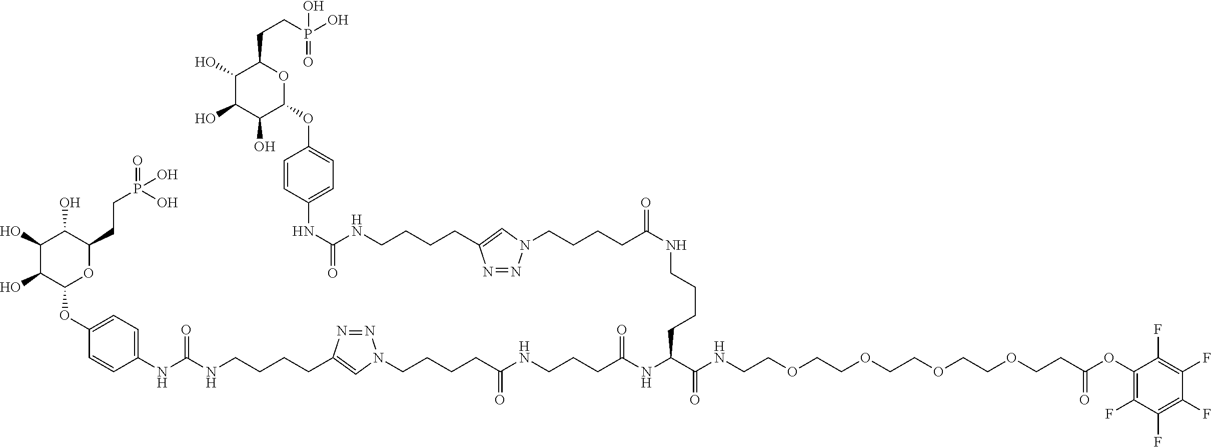 [SMILES: O=C(CCCCn1cc(CCCCNC(=O)Nc2ccc(O[C@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 724 (I-98) | 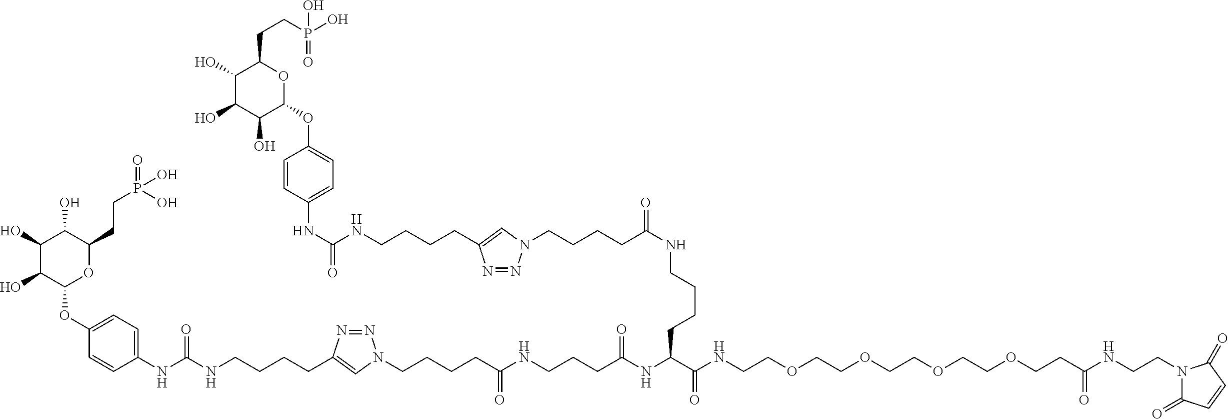 [SMILES: O=C(CCCCn1cc(CCCCNC(=O)Nc2ccc(O[C@H]3O[C@H](CCP(=O)(O)O)[C@@H](O)[C@H](O)[C@@H]3O)cc2)nn1)NCCCC[C@H](NC(=O)CCCNC(=O)CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)NCCN1C(=O)C=CC1=O] |
| 725 (I-99) | 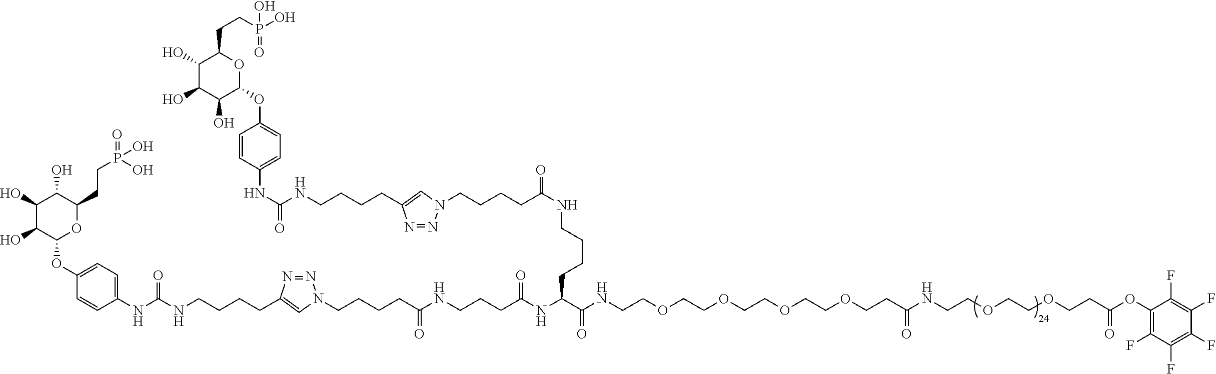 |
| 726 (I-100) | 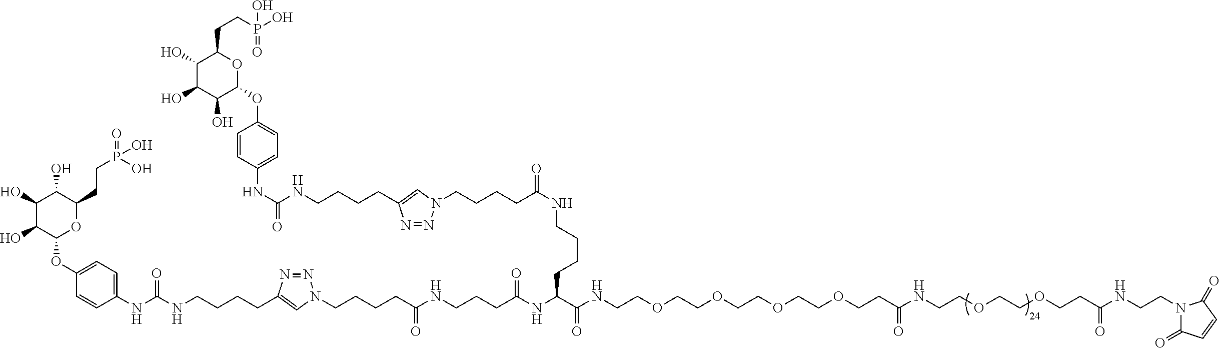 |
| 727 | 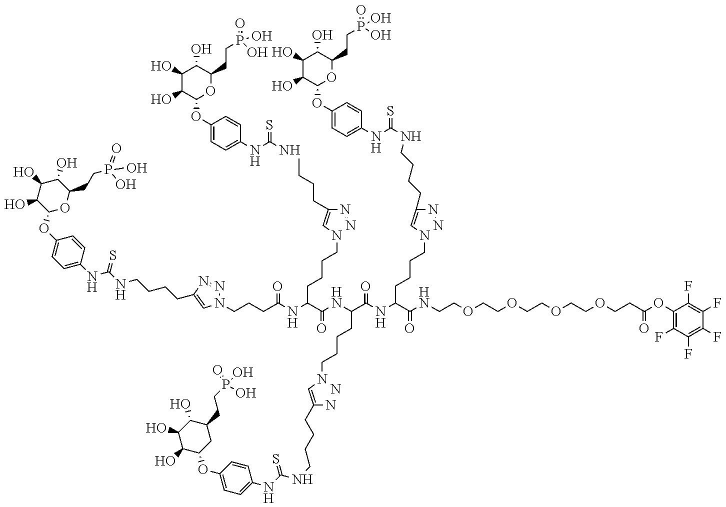 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NC(CCCCn1cc(CCCCNC(=S)Nc2ccc(O[C@H]3C[C@H](CCP(=O)(O)O)[C@@H](O)[C@H](O)[C@@H]3O)cc2)nn1)C(=O)NC(CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 728 (I-52) | 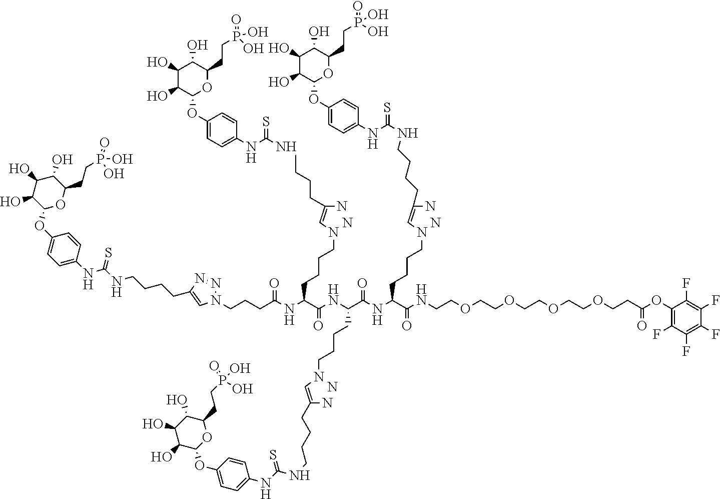 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 729 | 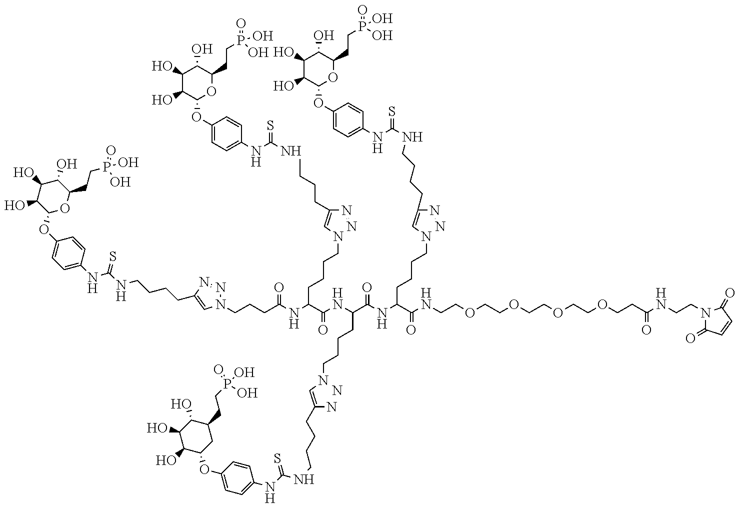 [SMILES: O=C(CCOCCOCCOCCOCCNC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)C(CCCCn1cc(CCCCNC(=S)Nc2ccc(O[C@H]3C[C@H](CCP(=O)(O)O)[C@@H](O)[C@H](O)[C@@H]3O)cc2)nn1)NC(=O)C(CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 730 (I-56) | 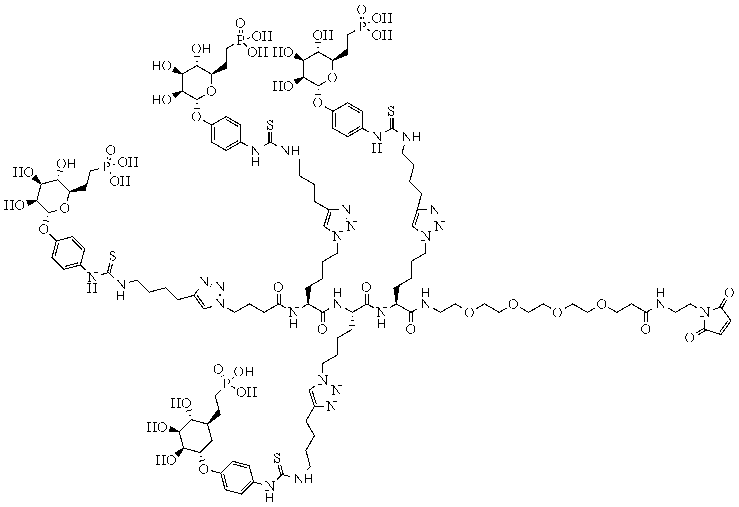 [SMILES: O=C(CCOCCOCCOCCOCCNC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3C[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)CCCn1cc(CCCCNC(=S)Nc2ccc(O[C@H]3O[C@H](CCP(=O)(O)O)[C@@H](O)[C@H](O)[C@@H]3O)cc2)nn1)NCCN1C(=O)C=CC1=O] |
| 731 | 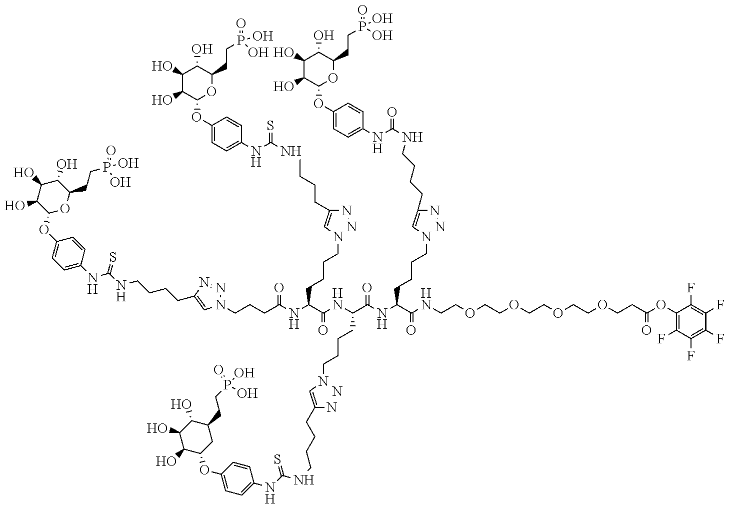 [SMILES: O=C(CCCn1cc(CCCCNC(=S)Nc2ccc(O[C@H]3O[C@H](CCP(=O)(O)O)[C@@H](O)[C@H](O)[C@@H]3O)cc2)nn1)N[C@@H](CCCCn1cc(CCCCNC(=S)Nc2ccc(O[C@H]3O[C@H](CCP(=O)(O)O)[C@@H](O)[C@H](O)[C@@H]3O)cc2)nn1)C(=O)N[C@@H](CCCCn1cc(CCCCNC(=S)Nc2ccc(O[C@H]3C[C@H](CCP(=O)(O)O)[C@@H](O)[C@H](O)[C@@H]3O)cc2)nn1)C(=O)N[C@@H](CCCCn1cc(CCCCNC(=O)Nc2ccc(O[C@H]3O[C@H](CCP(=O)(O)O)[C@@H](O)[C@H](O)[C@@H]3O)cc2)nn1)C(=O)NCCOCCOCCOCCOCCC(=O)Oc1c(F)c(F)c(F)c(F)c1F] |
| 732 (I-82) | 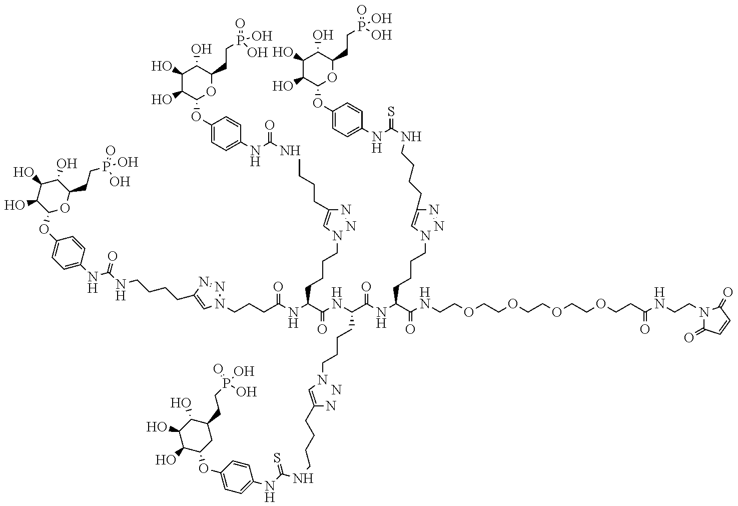 [SMILES: O=C(CCOCCOCCOCCOCCNC(=O)[C@H](CCCCn1cc(CCCCNC(=S)Nc2ccc(O[C@H]3O[C@H](CCP(=O)(O)O)[C@@H](O)[C@H](O)[C@@H]3O)cc2)nn1)NC(=O)[C@H](CCCCn1cc(CCCCNC(=S)Nc2ccc(O[C@H]3C[C@H](CCP(=O)(O)O)[C@@H](O)[C@H](O)[C@@H]3O)cc2)nn1)NC(=O)[C@H](CCCCn1cc(CCCCNC(=O)Nc2ccc(O[C@H]3O[C@H](CCP(=O)(O)O)[C@@H](O)[C@H](O)[C@@H]3O)cc2)nn1)NC(=O)CCCn1cc(CCCCNC(=O)Nc2ccc(O[C@H]3O[C@H](CCP(=O)(O)O)[C@@H](O)[C@H](O)[C@@H]3O)cc2)nn1)NCCN1C(=O)C=CC1=O] |

The present disclosure is meant to encompass stereoisomers of any one of the compounds described herein. In some instance, the compound includes an enantiomer of the D-mannopyrannose ring, or analog thereof.

In certain embodiments, the compound comprises a L-mannose ring analog and has the structure:

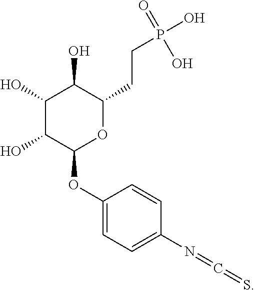 [SMILES: O=P(O)(O)CC[C@@H]1O[C@@H](Oc2ccc(N=C=S)cc2)[C@H](O)[C@H](O)[C@H]1O]

In certain embodiments, the compound comprises a L-mannose ring and has one of the following structures:

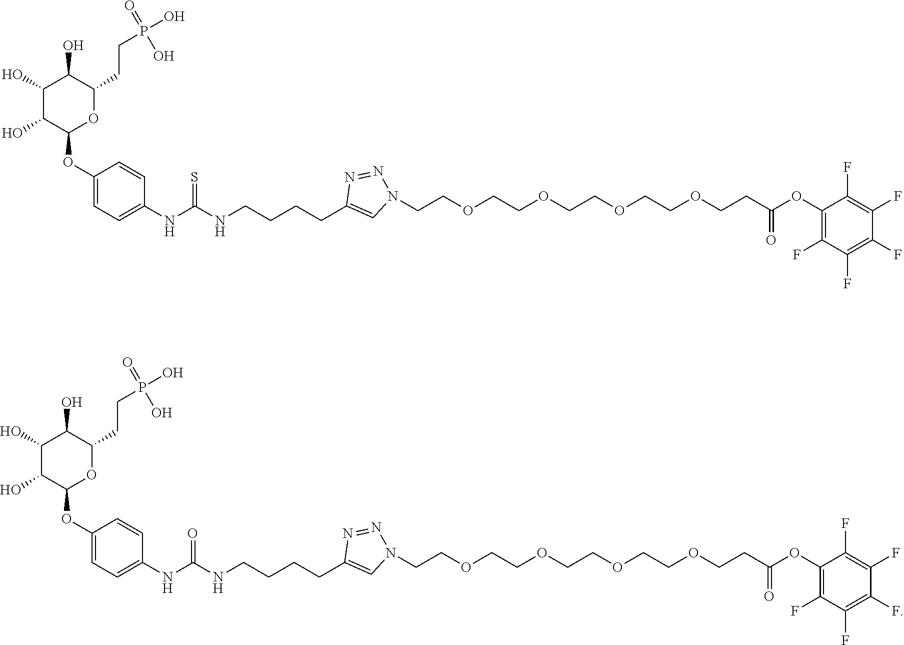 [SMILES: O=C(CCOCCOCCOCCOCCn1cc(CCCCNC(=S)Nc2ccc(O[C@@H]3O[C@@H](CCP(=O)(O)O)[C@H](O)[C@@H](O)[C@H]3O)cc2)nn1)Oc1c(F)c(F)c(F)c(F)c1F ; O=C(NCCCCc1cn(CCOCCOCCOCCOCCC(=O)Oc2c(F)c(F)c(F)c(F)c2F)nn1)Nc1ccc(O[C@@H]2O[C@@H](CCP(=O)(O)O)[C@H](O)[C@@H](O)[C@H]2O)cc1]

Exemplary ASGPR binding compounds of formula (Ib) are shown in Tables 8-9.

TABLE 8
ASGPR binding compounds of formula (Ib) and IIIj)

| # | Structure |
|---|---|
| 801 (I- 117) | 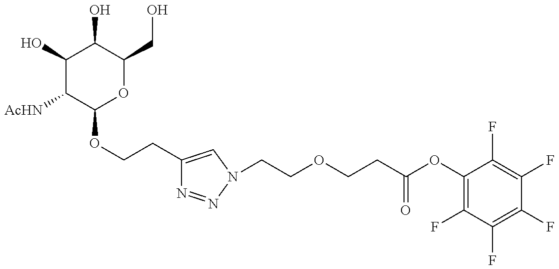 [SMILES: CC(=O)N[C@H]1[C@H](OCCc2cn(CCOCCC(=O)Oc3c(F)c(F)c(F)c(F)c3F)nn2)O[C@H](CO)[C@H](O)[C@@H]1O] |
| 802 (I- 115) | 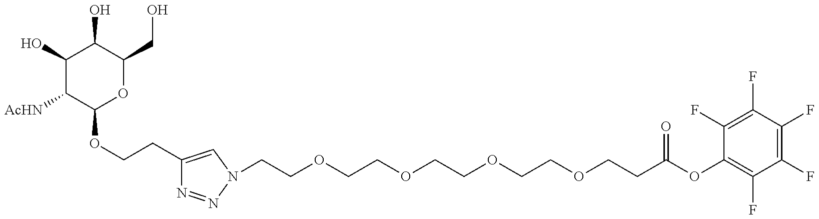 [SMILES: CC(=O)N[C@H]1[C@H](OCCc2cn(CCOCCOCCOCCOCCC(=O)Oc3c(F)c(F)c(F)c(F)c3F)nn2)O[C@H](CO)[C@H](O)[C@@H]1O] |
| 803 | 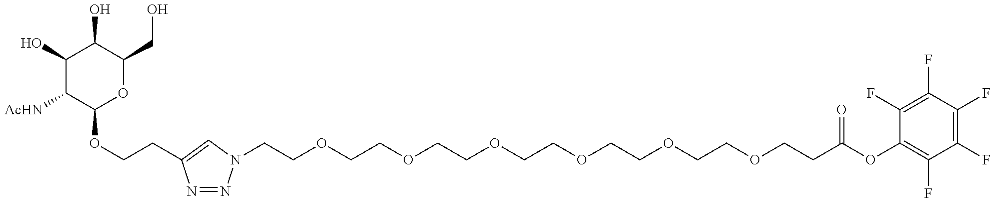 [SMILES: CC(=O)N[C@H]1[C@H](OCCc2cn(CCOCCOCCOCCOCCOCCOCCC(=O)Oc3c(F)c(F)c(F)c(F)c3F)nn2)O[C@H](CO)[C@H](O)[C@@H]1O] |
| 804 | 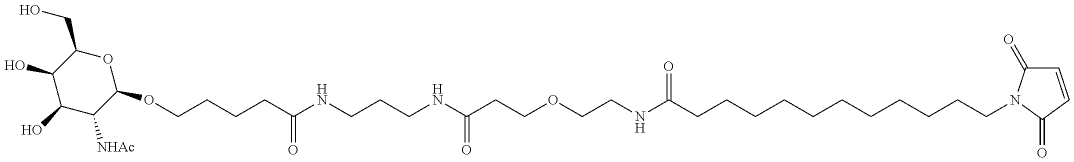 [SMILES: CC(=O)N[C@H]1[C@H](OCCCCC(=O)NCCCNC(=O)CCOCCNC(=O)CCCCCCCCCCCN2C(=O)C=CC2=O)O[C@H](CO)[C@H](O)[C@@H]1O] |
| 805 | 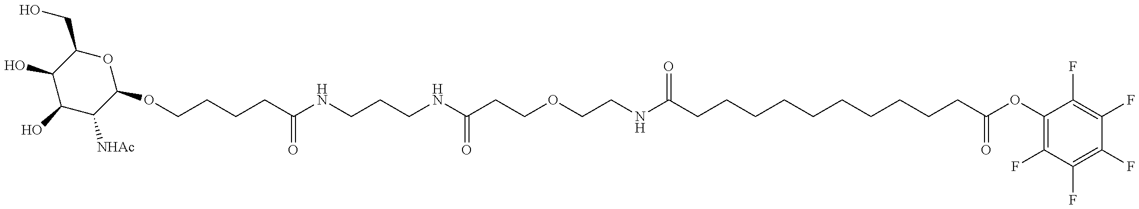 [SMILES: CC(=O)N[C@H]1[C@H](OCCCCC(=O)NCCCNC(=O)CCOCCNC(=O)CCCCCCCCCCC(=O)Oc2c(F)c(F)c(F)c(F)c2F)O[C@H](CO)[C@H](O)[C@@H]1O] |
| 806 (I- 112) | 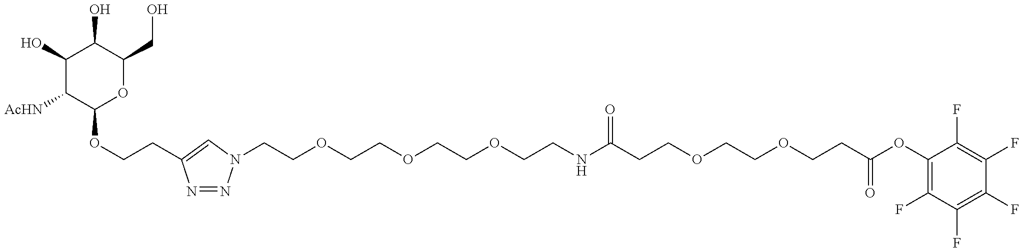 [SMILES: CC(=O)N[C@H]1[C@H](OCCc2cn(CCOCCOCCOCCNC(=O)CCOCCOCCC(=O)Oc3c(F)c(F)c(F)c(F)c3F)nn2)O[C@H](CO)[C@H](O)[C@@H]1O] |
| 807 | 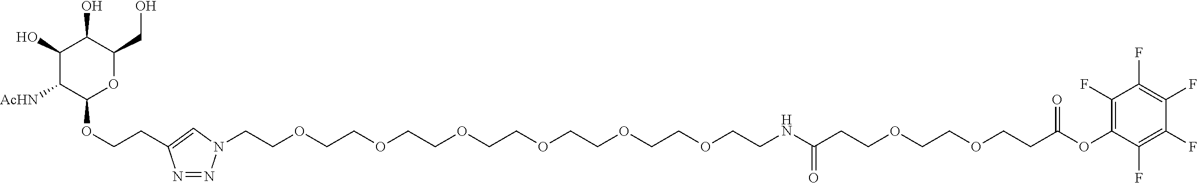 [SMILES: CC(=O)N[C@H]1[C@H](OCCc2cn(CCOCCOCCOCCOCCOCCOCCNC(=O)CCOCCOCCC(=O)Oc3c(F)c(F)c(F)c(F)c3F)nn2)O[C@H](CO)[C@H](O)[C@@H]1O] |
| 808 | 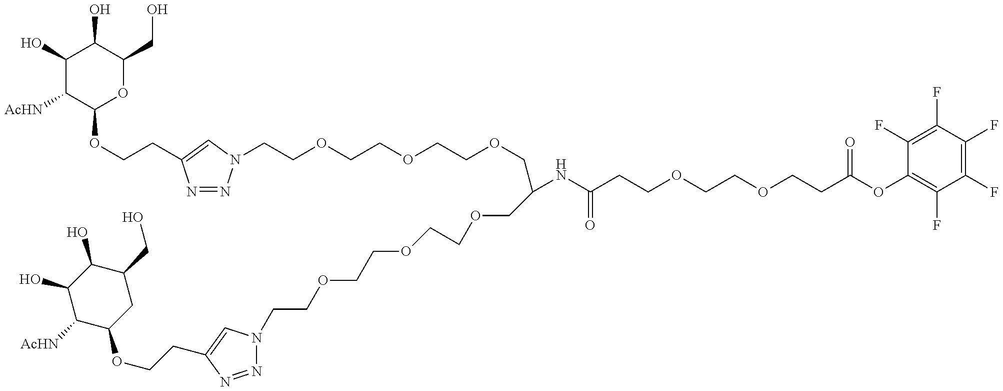 [SMILES: CC(=O)N[C@@H]1[C@@H](O)[C@@H](O)[C@@H](CO)C[C@H]1OCCc1cn(CCOCCOCCOCC(COCCOCCOCCn2cc(CCO[C@@H]3O[C@H](CO)[C@H](O)[C@H](O)[C@H]3NC(C)=O)nn2)NC(=O)CCOCCOCCC(=O)Oc2c(F)c(F)c(F)c(F)c2F)nn1] |
| 809 | 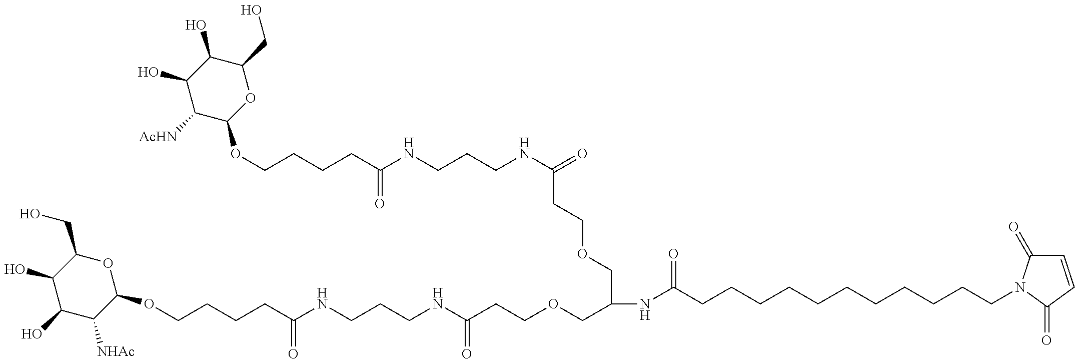 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 810 | 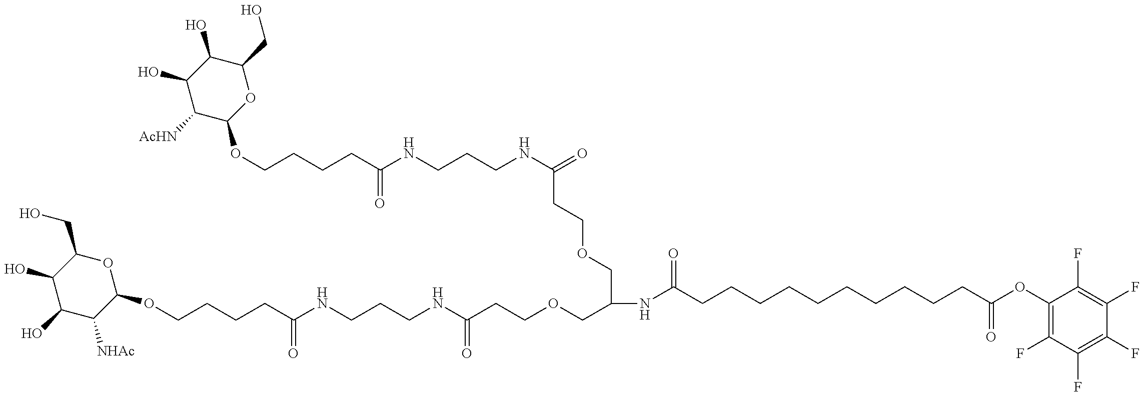 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 811 (I- 111) | 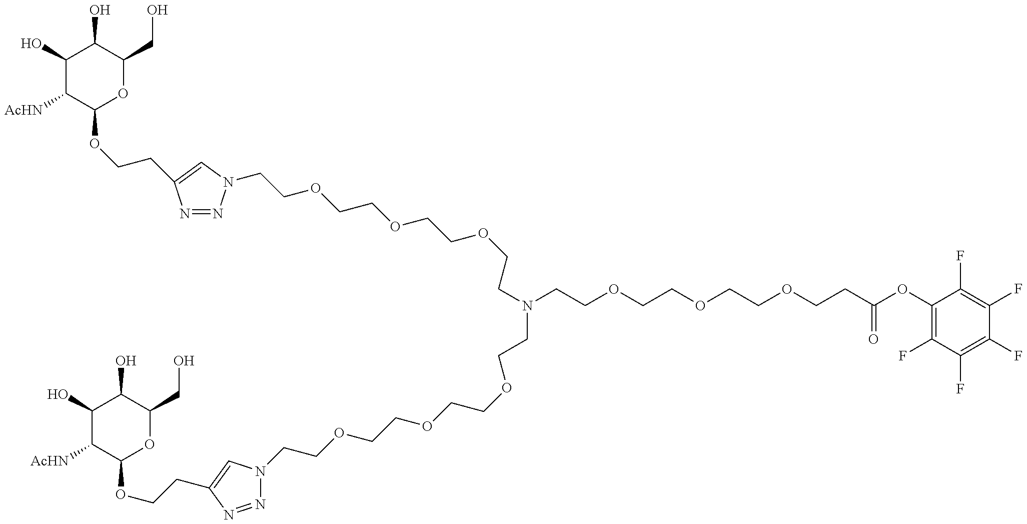 [SMILES: CC(=O)N[C@H]1[C@H](OCCc2cn(CCOCCOCCOCCN(CCOCCOCCOCCC(=O)Oc3c(F)c(F)c(F)c(F)c3F)CCOCCOCCOCCn3cc(CCO[C@@H]4O[C@H](CO)[C@H](O)[C@H](O)[C@H]4NC(C)=O)nn3)nn2)O[C@H](CO)[C@H](O)[C@@H]1O] |
| 812 (I- 127) | 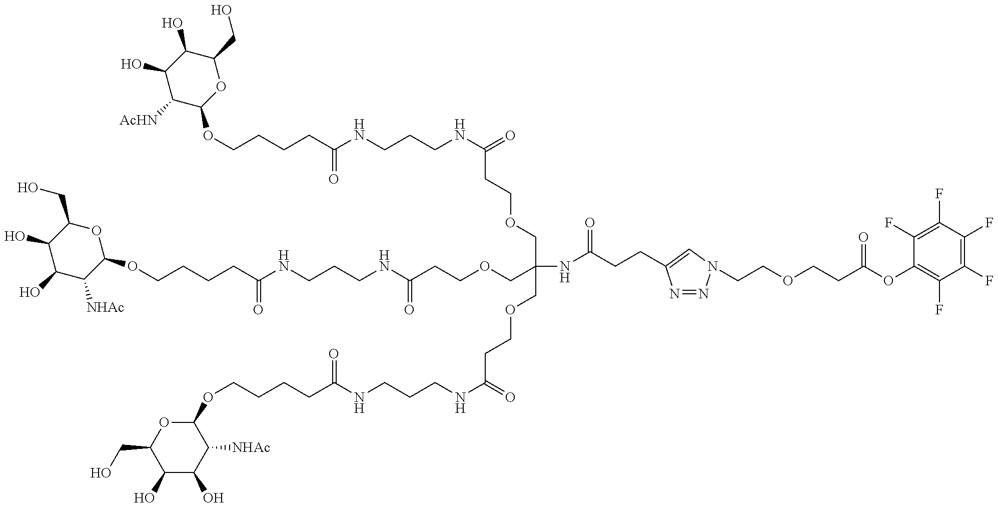 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCc2cn(CCOCCC(=O)Oc3c(F)c(F)c(F)c(F)c3F)nn2)O[C@H](CO)[C@H](O)[C@@H]1O] |
| 813 | 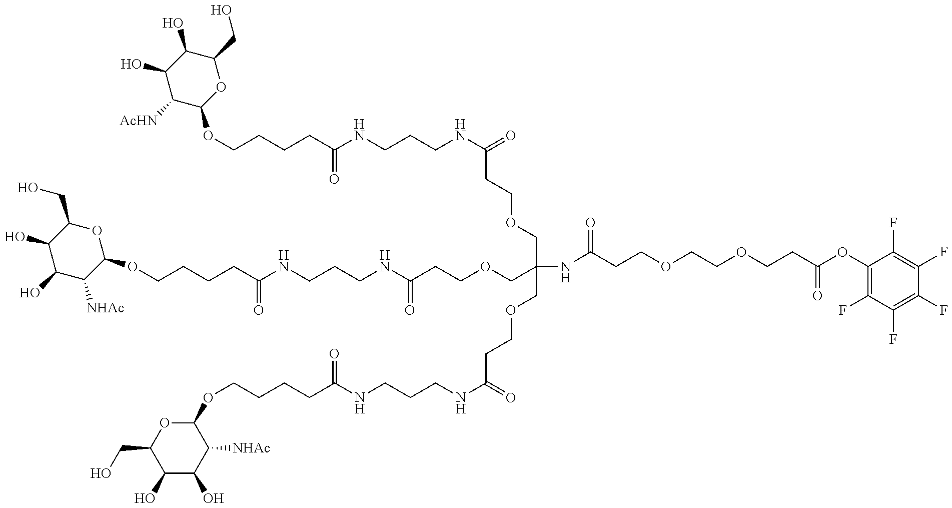 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCC(=O)Oc2c(F)c(F)c(F)c(F)c2F)O[C@H](CO)[C@H](O)[C@@H]1O] |
| 814 (I- 107) | 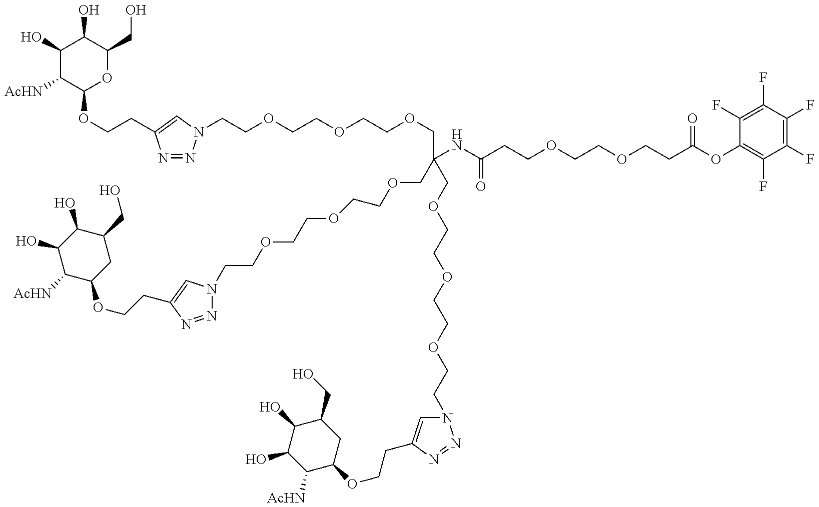 [SMILES: CC(=O)N[C@@H]1[C@@H](O)[C@@H](O)[C@@H](CO)C[C@H]1OCCc1cn(CCOCCOCCOCC(COCCOCCOCCn2cc(CCO[C@@H]3O[C@H](CO)[C@H](O)[C@H](O)[C@H]3NC(C)=O)nn2)(COCCOCCOCCn2cc(CCO[C@@H]3C[C@H](CO)[C@H](O)[C@H](O)[C@H]3NC(C)=O)nn2)NC(=O)CCOCCOCCC(=O)Oc2c(F)c(F)c(F)c(F)c2F)nn1] |
| 815 | 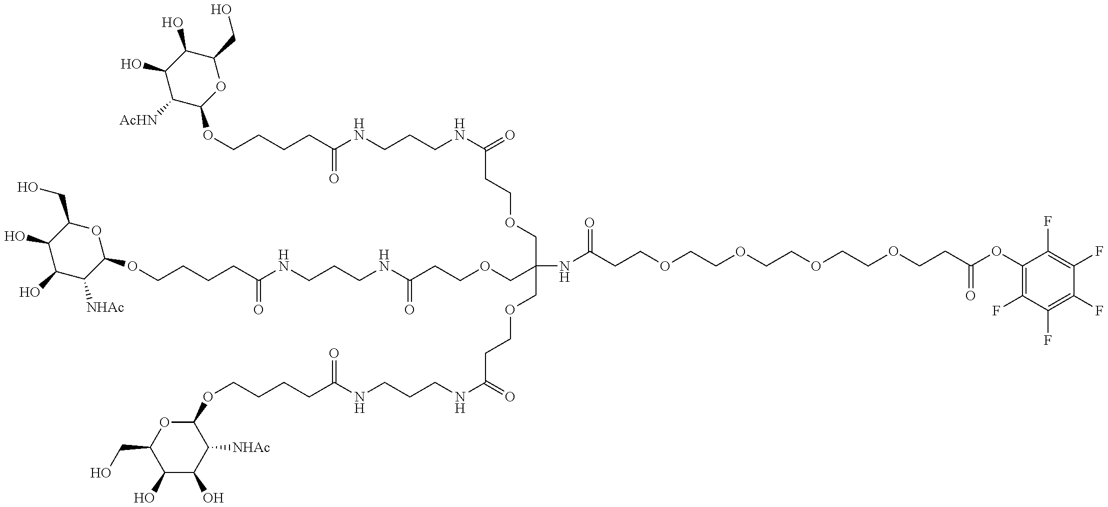 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCC(=O)Oc2c(F)c(F)c(F)c(F)c2F)O[C@H](CO)[C@H](O)[C@@H]1O] |
| 816 (I- 124) | 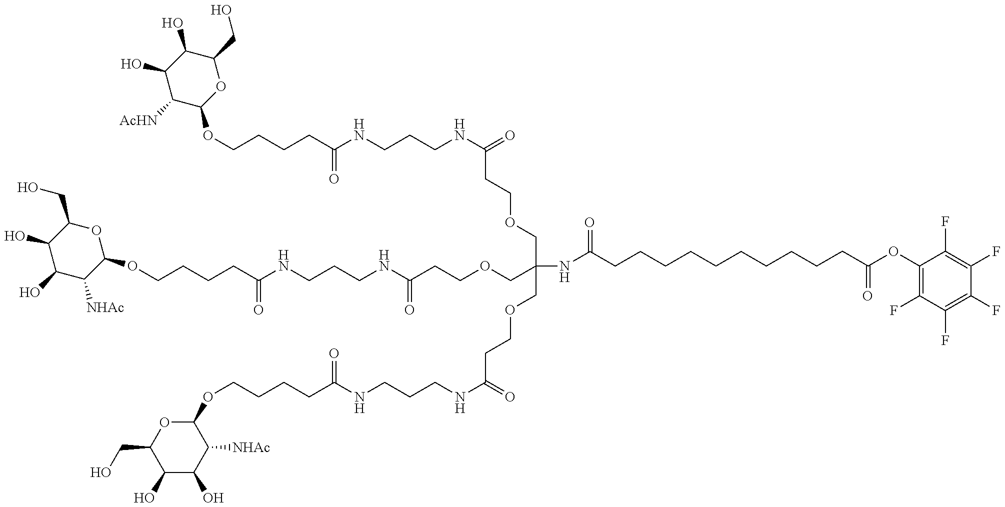 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCC(=O)Oc2c(F)c(F)c(F)c(F)c2F)O[C@H](CO)[C@H](O)[C@@H]1O] |
| 817 (I- 123) | 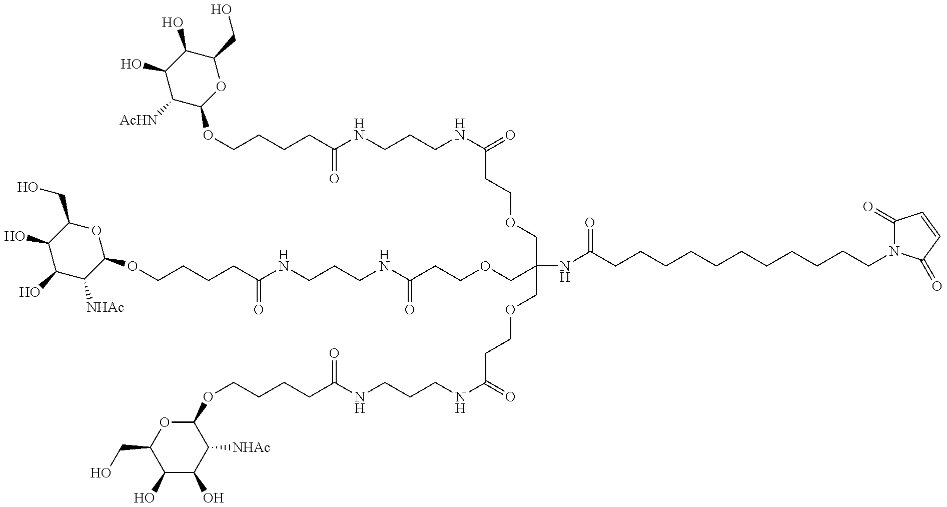 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCCCCCCCCCCN2C(=O)C=CC2=O)O[C@H](CO)[C@H](O)[C@@H]1O] |
| 818 (I- 129) | 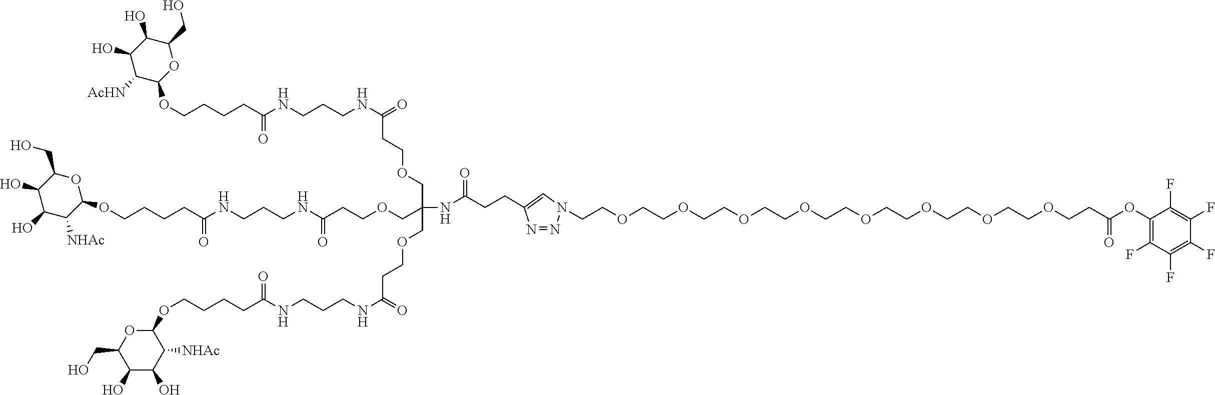 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCc2cn(CCOCCOCCOCCOCCOCCOCCOCCOCCC(=O)Oc3c(F)c(F)c(F)c(F)c3F)nn2)O[C@H](CO)[C@H](O)[C@@H]1O] |
| 819 | 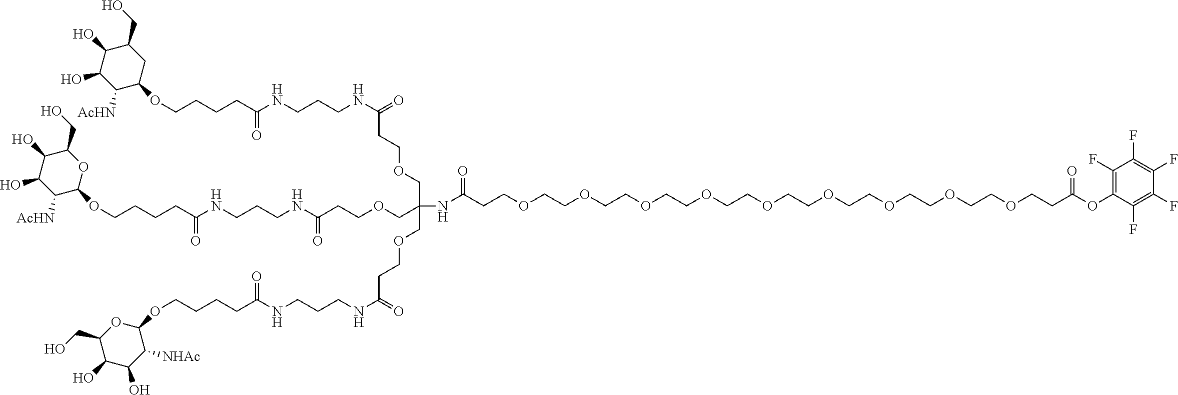 [SMILES: CC(=O)N[C@@H]1[C@@H](O)[C@@H](O)[C@@H](CO)C[C@H]1OCCCCC(=O)NCCCNC(=O)CCOCC(COCCC(=O)NCCCNC(=O)CCCCO[C@@H]1O[C@H](CO)[C@H](O)[C@H](O)[C@H]1NC(C)=O)(COCCC(=O)NCCCNC(=O)CCCCO[C@@H]1O[C@H](CO)[C@H](O)[C@H](O)[C@H]1NC(C)=O)NC(=O)CCOCCOCCOCCOCCOCCOCCOCCOCCOCCC(=O)Oc1c(F)c(F)c(F)c(F)c1F] |
| 820 | 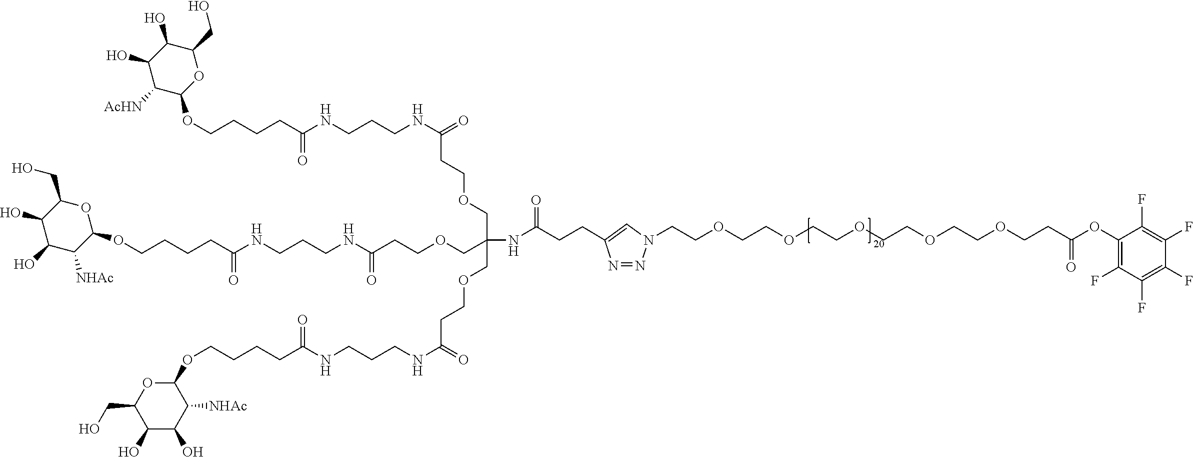 |
| 821 (I- 135) | 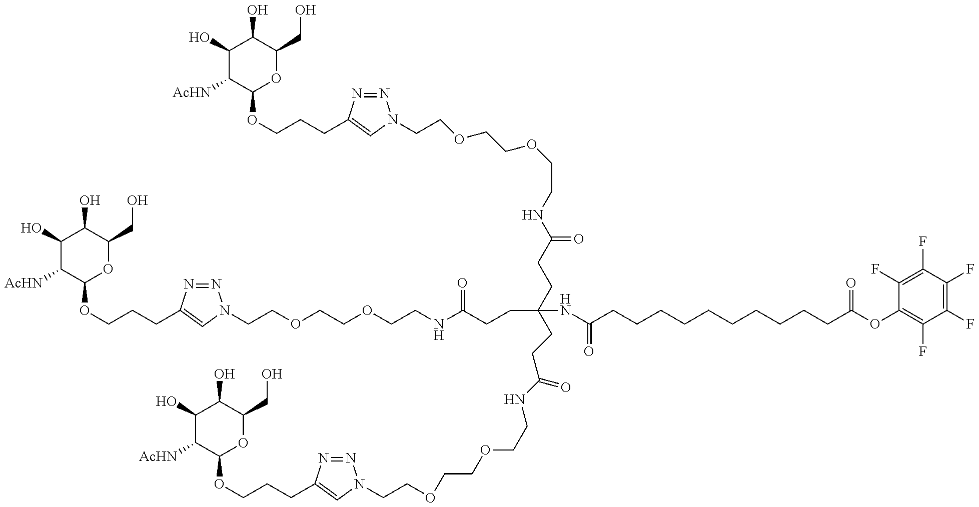 [SMILES: CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](O)[C@H]4NC(C)=O)nn3)(CCC(=O)NCCOCCOCCn3cc(CCCO[C@@H]4O[C@H](CO)[C@H](O)[C@H](O)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1O] |

TABLE 9
ASGPR binding compounds of formula (Ib) and IIIk)

| # | Structure |
|---|---|
| 901 (I-118) | 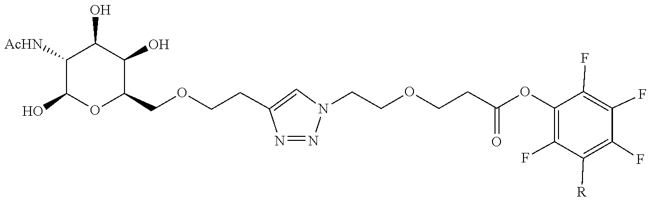 |
| 902 (I-116) | 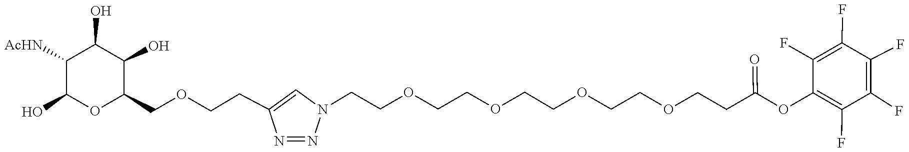 [SMILES: CC(=O)N[C@@H]1[C@@H](O)[C@@H](O)[C@@H](COCCc2cn(CCOCCOCCOCCOCCC(=O)Oc3c(F)c(F)c(F)c(F)c3F)nn2)O[C@H]1O] |
| 903 (I-113) | 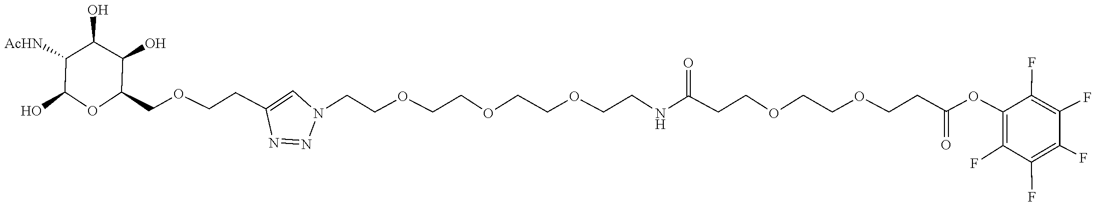 [SMILES: CC(=O)N[C@@H]1[C@@H](O)[C@@H](O)[C@@H](COCCc2cn(CCOCCOCCOCCNC(=O)CCOCCOCCC(=O)Oc3c(F)c(F)c(F)c(F)c3F)nn2)O[C@H]1O] |
| 904 (I-110) | 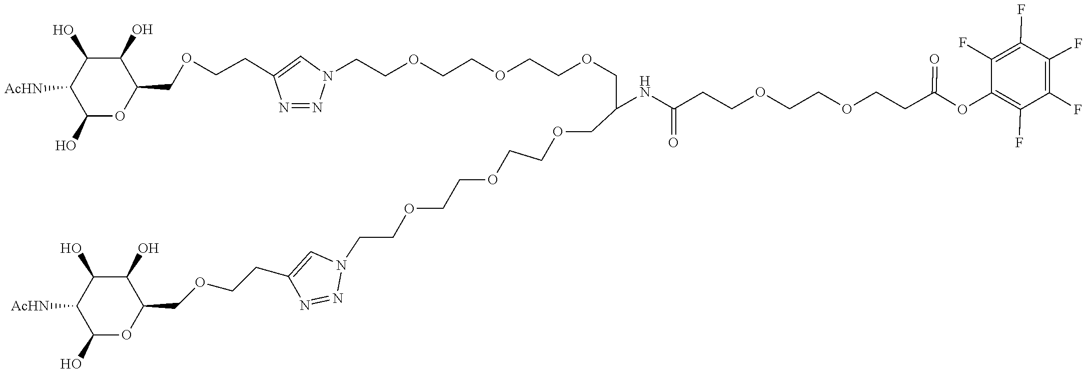 [SMILES: CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |
| 905 (I-108) | 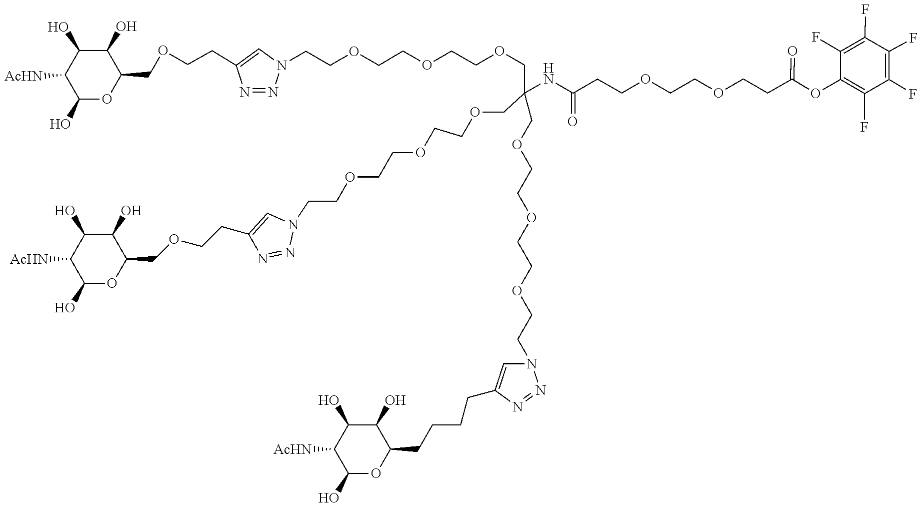 [SMILES: CC(=O)N[C@@H]1[C@@H](O)[C@@H](O)[C@@H](CCCCc2cn(CCOCCOCCOCC(COCCOCCOCCn3cc(CCOC[C@H]4O[C@@H](O)[C@H](NC(C)=O)[C@@H](O)[C@H]4O)nn3)(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O] |
| 906 (I-136) | 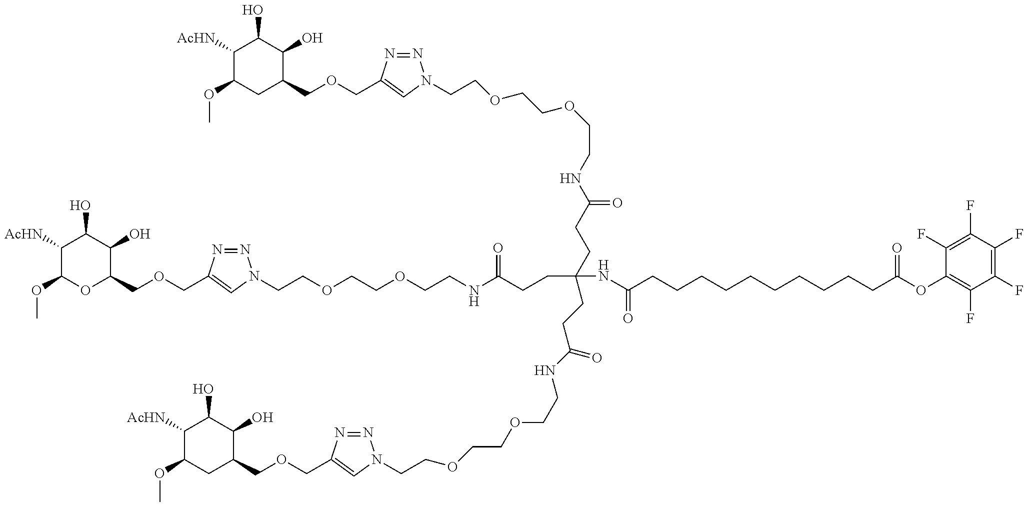 [SMILES: CO[C@@H]1O[C@H](COCc2cn(CCOCCOCCNC(=O)CCC(CCC(=O)NCCOCCOCCn3cc(COC[C@H]4C[C@@H](OC)[C@H](NC(C)=O)[C@@H](O)[C@H]4O)nn3)(CCC(=O)NCCOCCOCCn3cc(COC[C@H]4C[C@@H](OC)[C@H](NC(C)=O)[C@@H](O)[C@H]4O)nn3)NC(=O)CCCCCCCCCCC(=O)Oc3c(F)c(F)c(F)c(F)c3F)nn2)[C@H](O)[C@H](O)[C@H]1NC(C)=O] |

### Conjugates

The compounds of this disclosure can be referred to as a conjugate, e.g., when the moiety of interest (Y) is a molecule (e.g., as described herein). Such conjugates can be prepared by conjugation of a chemoselective ligation group of any one of the compounds described herein with a compatible reactive group of a molecule Y. The compatible group of the molecule Y can be introduced by modification prior to conjugation, or can be a group present in the molecule. Alternatively, such conjugates can be prepared de novo, e.g., via modification of a Y molecule of interest starting material to introduce a linker, e.g., to which a ligand X can be attached.

Aspects of this disclosure include compounds of formula (I) where the moiety of interest Y is a selected from small molecule, dye, flurorophore, monosaccharide, disaccharide, trisaccharide, and biomolecule. In some embodiments, Y is a small molecule that specifically binds to a target molecule, such as a target protein.

In some embodiments of the compounds of this disclosure, Y is a biomolecule. In some embodiments, the biomolecule is selected from protein, polynucleotide, polysaccharide, peptide, glycoprotein, lipid, enzyme, antibody, and antibody fragment. In some embodiments, Y is a biomolecule that specifically binds to a target molecule, such as a target protein.

The compounds of this disclosure can, in some cases, be referred to as a conjugate, e.g., when the moiety of interest (Y) is a molecule such as a biomolecule, where the conjugate can derived from a conjugation or coupling reaction between a chemoselective ligation group and a compatible group on the biomolecule. In some embodiments, the biomolecule is conjugated via a naturally occurring group of the biomolecule. In some embodiments, the biomolecule is conjugated via a compatible functional group that is introduced into the biomolecule prior to chemoselective conjugation. In such cases, the linking moiety between X and Y incorporates the residual group (e.g., Z) that is the product of the chemoselective ligation chemistry.

Aspects of this disclosure include compounds of formula (Ia) where the moiety of interest Y is a moiety that specifically binds to a target molecule, such as a target protein. The target protein can be the target protein is a membrane bound protein or an extracellular protein. In some embodiments of the compounds of this disclosure, Y is a biomolecule that specifically binds to a target protein. This disclosure provides conjugates of the particular M6PR or ASGPR binding compounds and conjugates. In some embodiments, the conjugate includes a moiety of interest Y that specifically binds a target protein, and can find use in methods of cell uptake or internalization of the target protein via binding to the cell surface receptor, and eventual degradation of the target protein.

In some embodiments, Y is an aptamer that specifically binds to a target molecule, such as a target protein. In some embodiments, Y is a peptide or protein (e.g., peptidic binding motif, protein domain, engineered polypeptide, or glycoprotein) that specifically binds to a target molecule, such as a target protein. In some embodiments, Y is an antibody or antibody fragment that specifically binds to a target molecule, such as a target protein. In some embodiments, Y is a polynucleotide or oligonucleotide that specifically binds to a target molecule, such as a target protein or a target nucleic acid.

In some embodiments, one Y biomolecule is conjugated to a single moiety (X) that specifically binds to the cell surface receptor (e.g., M6PR or ASGPR) via a linker L. In some embodiments, one Y biomolecule is conjugated to one (Xₙ-L)- group, wherein when n=1 the (Xₙ-L)- group is referred to as monovalent, and when n>1 the (Xₙ-L)- group is referred to as multivalent (e.g., bivalent, trivalent, etc.). It is understood that in some embodiments of formula (Ia), where Y is a biomolecule, Y can be conjugated to two or more (Xₙ-L)- groups, wherein each (Xₙ-L)- group may itself be monovalent or multivalent (e.g., bivalent, trivalent, etc.). In such cases, the ratio of linked (Xₙ-L)- groups to biomolecule can be referred to as 2 or more.

Accordingly, provided herein are conjugates of the following formula (IVa):

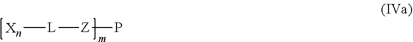

or a pharmaceutically acceptable salt thereof,
wherein:

X is a moiety that binds to a cell surface M6PR (e.g., as described herein) or a moiety that binds to a cell surface ASGPR (e.g., as described herein);

L is a linker (e.g., as described herein);

n is an integer of 1 to 500 (e.g., 1 to 5);

m is an integer from 1 to 80; and

Z is a residual moiety resulting from the covalent linkage of a chemoselective ligation group (Y) to P;

P is a biomolecule (e.g., a biomolecule that specifically binds a target protein as described herein).

In some embodiments of formula (IVa), L is a linker of formula (IIa)-(IId) (e.g., as described herein). In some embodiments of formula (IVa), Xn-L-Z is derived from a compound of formula (XI)-(XVIa) (e.g., as described herein), where Y is a chemoselective ligation group.

In formula (IVa), Z can be any convenient residual moiety that results from the covalent linkage or conjugation of a chemoselective ligation group (Y) to a compatible reactive group of a biomolecule (P). In some instances, the compatible reactive group of biomolecule (P) is a group that can naturally be part on the biomolecule. In some instances, the compatible reactive group of biomolecule (P) is one that is introduced or incorporated into the biomolecule prior to conjugation. In such cases, the biomolecule (P) can be a modified version of a biomolecule. For example, a functional group (e.g., an amino group, a carboxylic acid group or a thiol group) of a biomolecule can be modified (e.g., using a chemical reagent such as 2-haloacetyl reagent, or 2-iminothiolane, or the like, or via coupling of a linker group including a chemoselective ligation group, such as an azide, alkyne, or the like) to introduce a compatible chemoselective ligation group.

In some embodiments of formula (IVa), L is a linker of formula (IIa) (e.g., as described herein). In certain embodiments of formula (IVa), Z is selected from the group consisting of

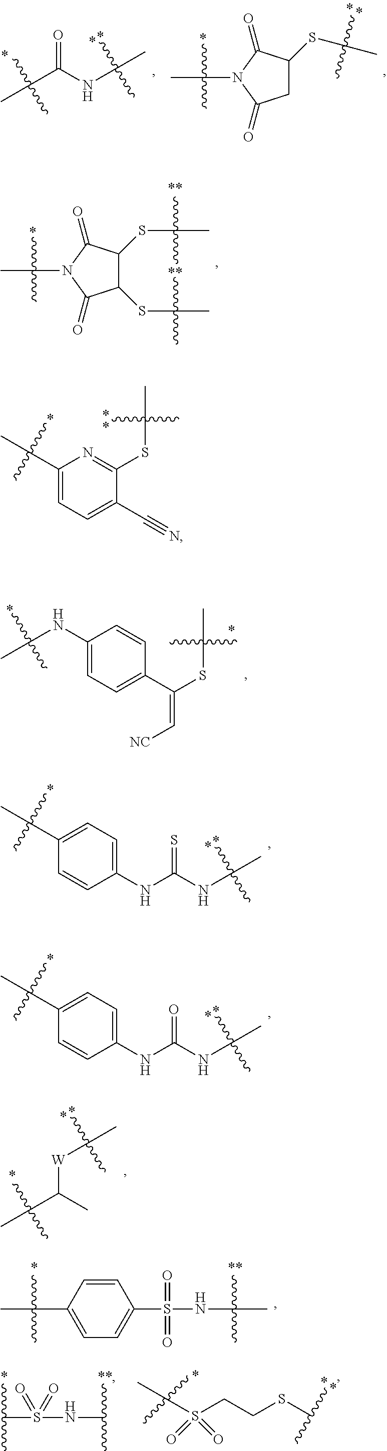

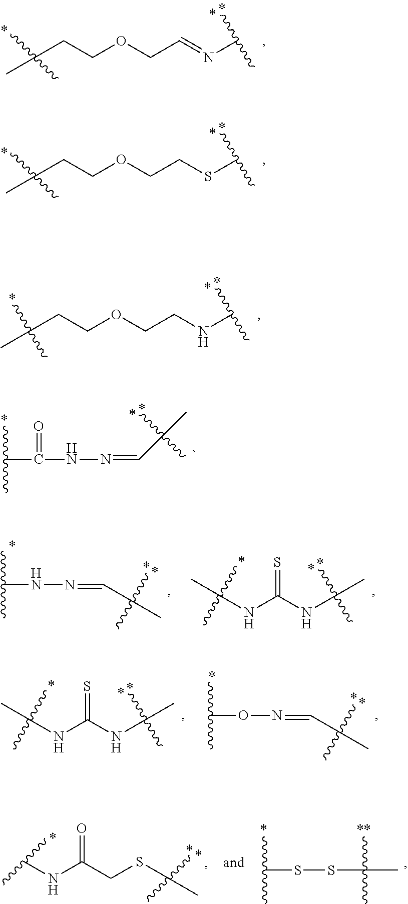

wherein [symbol] represents the point of attachment to the linker L,
wherein [symbol] represents the point of attachment to P,

### W is CH₂, N, O or S; and

P is a polypeptide.

In certain embodiments of formula (IVa), Z is selected from the group consisting of

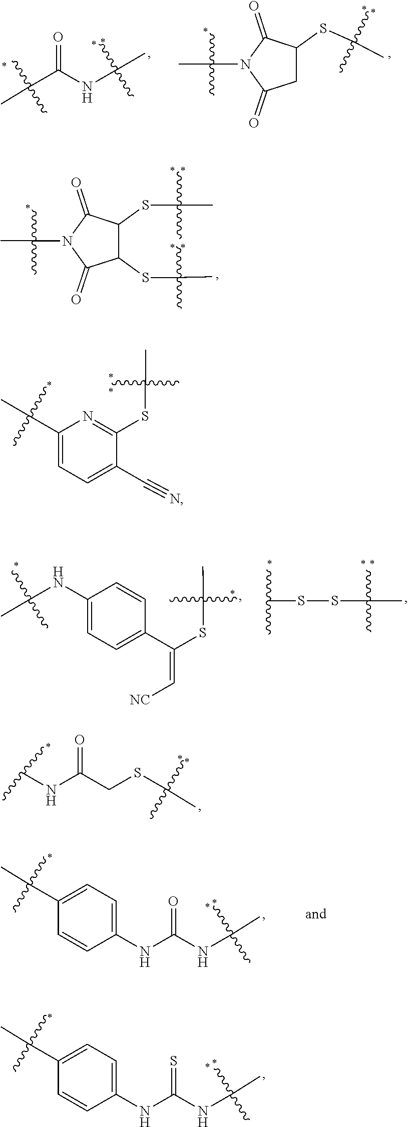

wherein [symbol] represents the point of attachment to L,
wherein [symbol] represents the point of attachment to P; and
P is a polypeptide.

In certain embodiments of formula (IVa), Z is selected from the group consisting of

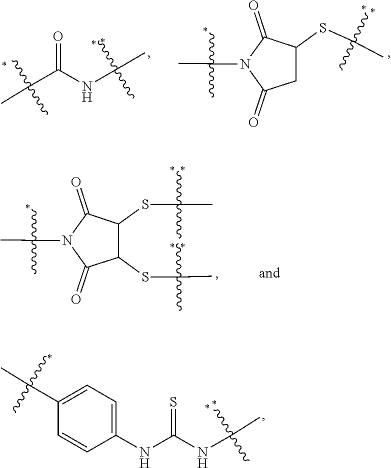

wherein [symbol] represents the point of attachment to L, wherein [symbol] represents the point of attachment to P. In some embodiments, P is a polypeptide.

In certain embodiments, n is 1. In certain embodiments, n is 2. In certain embodiments, n is 3. In certain embodiments, n is 4. In certain embodiments, n is 5.

In yet another aspect, provided herein are conjugates of formula (IVa) wherein L is a linker of the following formula (IIe)

-[(L¹)-(L²)-(L³)]ₙ-(L⁴)-(L⁵)- (IIe),

wherein L¹, L², L³, L⁴, L⁵, and n are defined herein.

In certain embodiments, L is selected from the linkers of Tables 1-2. In certain embodiments, L is selected from the linkers of Tables 1-2.

In another aspect, provided herein are conjugates of the following formula (IVb):

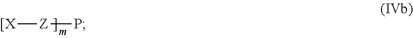

wherein:

X is a moiety that binds to a cell surface M6PR (e.g., as described herein) or a moiety that binds to a cell surface ASGPR (e.g., as described herein);

m is an integer from 1 to 80;

Z is a moiety having structure of

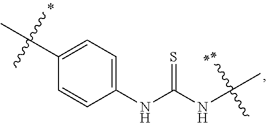

wherein [symbol] represents the point of attachment to X, wherein [symbol] represents the point of attachment to P; and P is a biomoelceule (e.g., as described herein, such as a polypeptide).

In certain embodiments of the conjugate of formulas (IVa) or (IVb), P is a peptide or protein, as defined herein.

In certain embodiments of the conjugate of formulas (IVa) or (IVb), P is selected from antibody, antibody fragment (e.g., antigen-binding fragment of an antibody), chimeric fusion protein, an engineered protein domain, D-protein binder of target protein, and peptide.

In certain embodiments of the conjugate of formulas (IVa) or (IVb), P is an antibody or antibody fragment (Ab), as defined herein.

Accordingly, in another aspect, provided herein are conjugates of the following formula (Va):

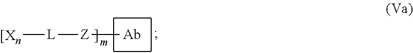

or a pharmaceutically acceptable salt thereof,
wherein:

X is a moiety that binds to a cell surface M6PR (e.g., as described herein) or a moiety that binds to a cell surface ASGPR (e.g., as described herein);

L is a linker (e.g., as described herein);

n is an integer of 1 to 5;

m is an integer from 1 to 80; and

wherein Z is a residual moiety resulting from the covalent linkage of a chemoselective ligation group (Y) to a compatible group of

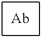

where Ab is an antibody or antibody fragment.

In some embodiments of formula (Va), L is a linker of formula (IIa) (e.g., as described herein).

In certain embodiments formula (Va), Z is selected from the group consisting of

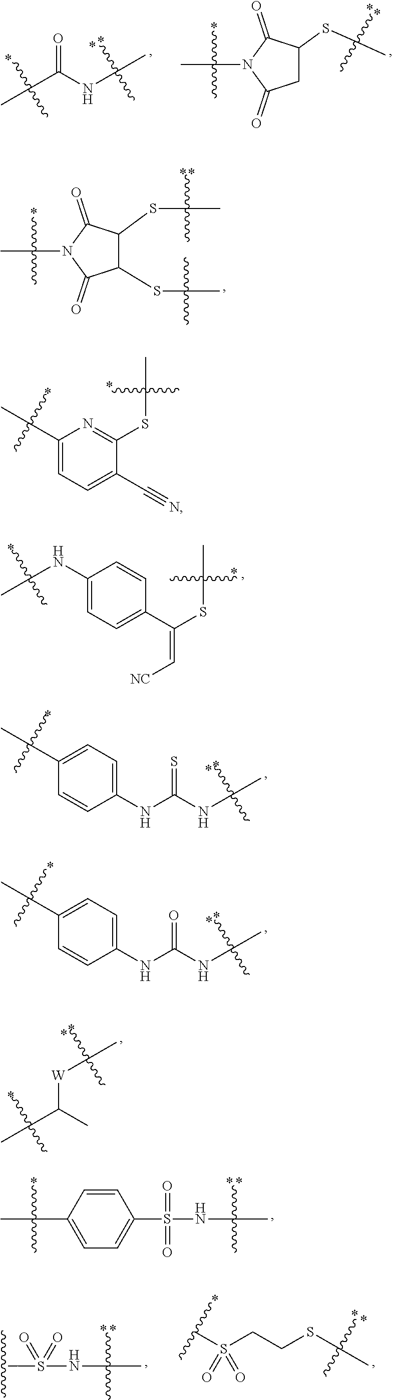

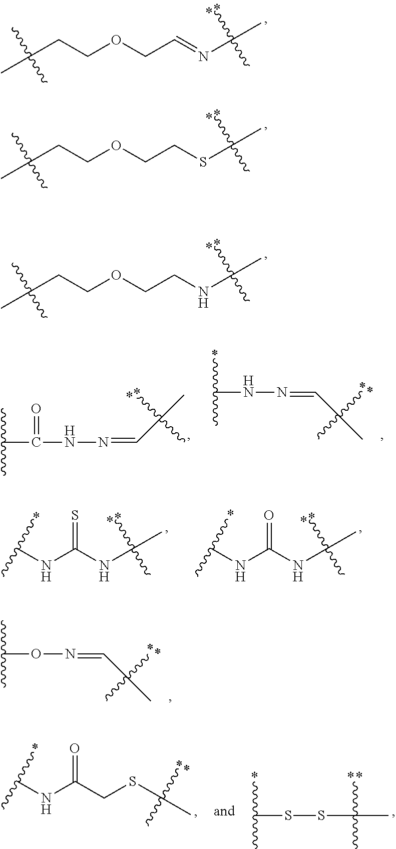

wherein [symbol] represents the point of attachment to L,
wherein [symbol] represents the point of attachment to

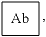

### W is CH₂, N, O or S; and

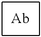

is an antibody.

In certain embodiments Z is selected from the group consisting of

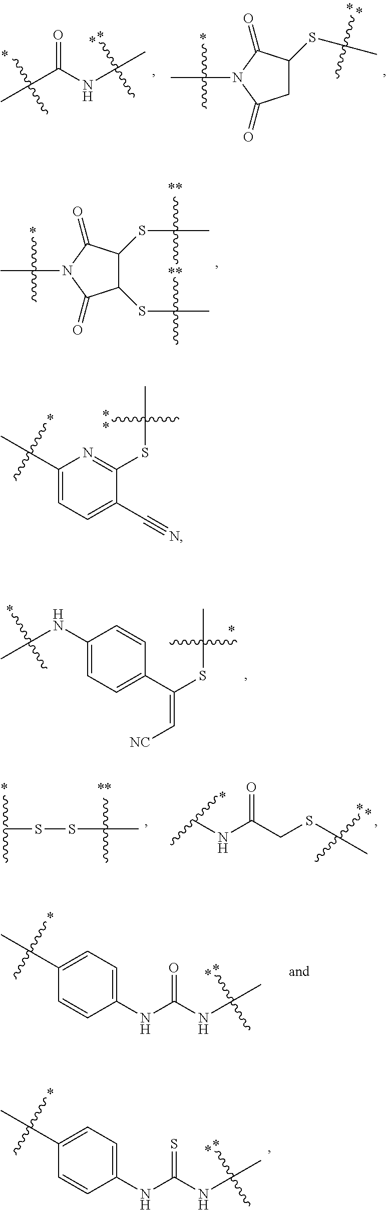

wherein [symbol] represents the point of attachment to L,
wherein [symbol] represents the point of attachment to

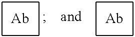

is an antibody.

In certain embodiments Z is selected from the group consisting of

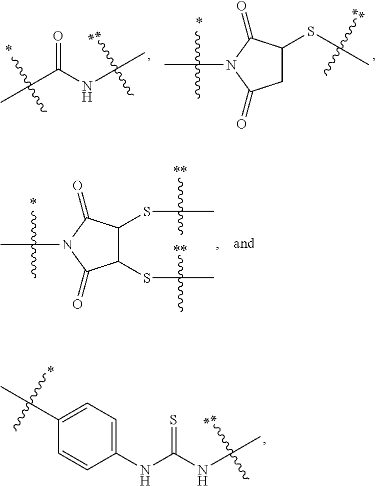

wherein [symbol] represents the point of attachment to L, wherein [symbol] represents the point of attachment to

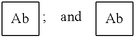

is an antibody.

In another aspect, provided herein are conjugates of the following formula (Va):

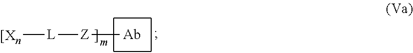

or a pharmaceutically acceptable salt thereof, wherein:

X is a moiety that binds to a cell surface M6PR (e.g., as described herein) or a moiety that binds to a cell surface ASGPR (e.g., as described herein);

L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa); and

wherein

each L¹ is independently

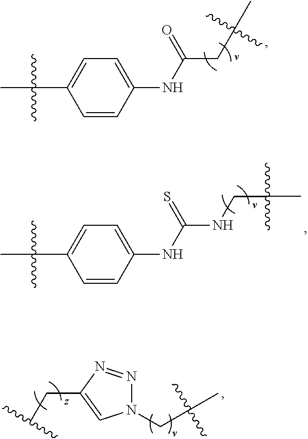

—C₁₋₆-alkylene-, —(OCH₂CH₂)ₖ—, or —(OCH₂CH₂)ₖ—(CH₂)ᵥ—;

each L³ is independently

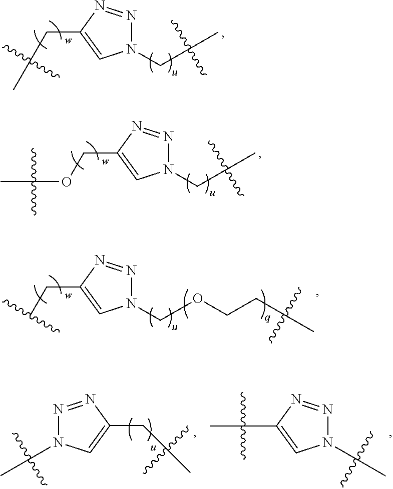

or —(OCH₂CH₂)_{q}—;

each L⁴ is independently —OCH₂CH₂—,

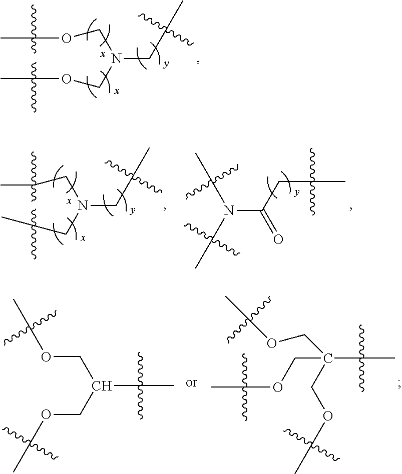

each L⁵ is independently —NHCO—C₁₋₆-alkylene- or —(OCH₂CH₂)ᵣ—;

each L⁶ is independently —NHCO—C₁₋₆-alkylene- or —(OCH₂CH₂)ₛ—;

each L⁷ is independently —NHCO—C₁₋₆-alkylene-, —(OCH₂CH₂)ₜ—, or —OCH₂—;

p, q, r, s, and t are each independently an integer of 1 to 20; a is 1 or 2; b, c, d, e, f,

and g are each independently 0, 1, or 2; u, v, w, x, y, and z are each independently

an integer of 1 to 10;

n is an integer of 1 to 5; wherein when d is O, n is 1, when d is 1, n is an integer of 1 to 3, and

when d is 2, n is an integer of 1 to 5;

m is an integer from 1 to 80;
Z is selected from the group consisting of

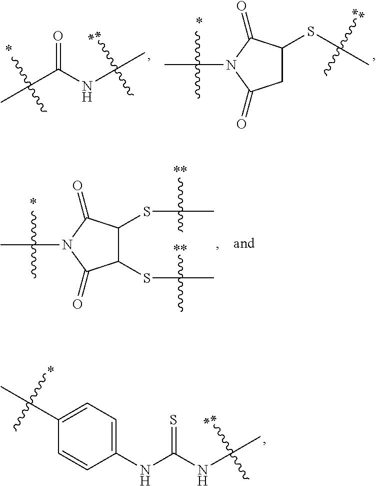

wherein [symbol] represents the point of attachment to L, wherein [symbol] represents the point of attachment to

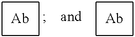

is an antibody.

In another aspect, provided herein are conjugates of the following formula (Va):

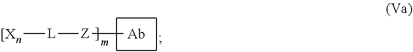

or a pharmaceutically acceptable salt thereof, wherein:

X is a moiety that binds to a cell surface M6PR (e.g., as described herein) or a moiety that binds to a cell surface ASGPR (e.g., as described herein);

L is a linker of the following formula (IIb):

-(L¹)ₐ-(L²)_{b}-(L³)_{c}- (IIb); and

wherein

L¹ is

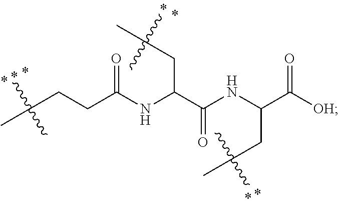 [SMILES: *CCC(=O)NC(C*)C(=O)NC(C*)C(=O)O]

L² is —(OCH₂CH₂)ₚ—;

L³ is —NHCO—C₁₋₆-alkylene-;

p is an integer of 1 to 20; a is 1, and b and c are each independently 0 or 1;

n is 2;

m is an integer from 1 to 80;

Z is selected from the group consisting of

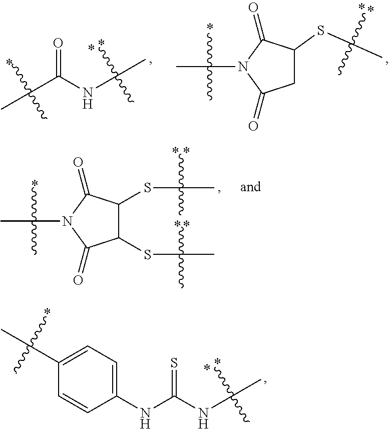

wherein [symbol] represents the point of attachment to L, wherein [symbol] represents the point of attachment to

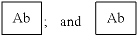

is an antibody.

In another aspect, provided herein are conjugates of the following formula (Va):

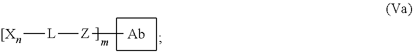

or a pharmaceutically acceptable salt thereof, wherein:

X is a moiety that binds to a cell surface M6PR (e.g., as described herein) or a moiety that binds to a cell surface ASGPR (e.g., as described herein);

L is a linker of the following formula (IIb):

-(L¹)ₐ-(L²)_{b}-(L³)_{c}- (IIb); and

wherein

L¹ is

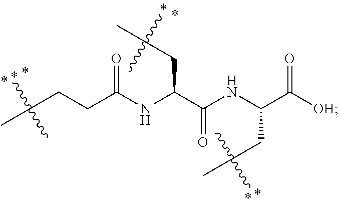 [SMILES: *CCC(=O)N[C@@H](C*)C(=O)N[C@@H](C*)C(=O)O]

L² is —(OCH₂CH₂)ₚ—;

L³ is —NHCO—C₁₋₆-alkylene-;

p is an integer of 1 to 20; a is 1, and b and c are each independently 0 or 1; n is 2;

m is an integer from 1 to 80;
Z is selected from the group consisting of

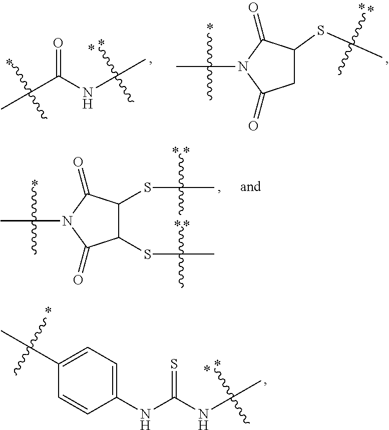

wherein [symbol] represents the point of attachment to L, wherein [symbol] represents the point of attachment to

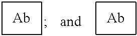

is an antibody.

In another aspect, provided herein are conjugates of the following formula (Va):

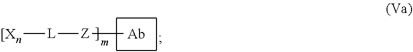

or a pharmaceutically acceptable salt thereof, wherein:
X is a moiety that binds to a cell surface M6PR (e.g., as described herein) or a moiety that binds to a cell surface ASGPR (e.g., as described herein);
L is a linker of the following formula (IIc):

-(L¹)ₐ-(L²)_{b}-(L³)_{c}-(L⁴)_{d}- (IIc); and

wherein
L¹ is

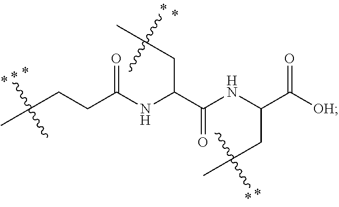 [SMILES: *CCC(=O)NC(C*)C(=O)NC(C*)C(=O)O]

L² is

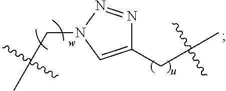

L³ is —NHCO—C₁₋₆-alkylene- or —(OCH₂CH₂)ₚ—;
L⁴ is —NHCO—C₁₋₆-alkylene- or —(OCH₂CH₂)_{q}—;
p and q are each independently an integer of 1 to 20; a is 1, and b, c, and d are each independently 0 or 1; and w and u are each independently an integer of 1 to 10;
wherein [symbol] represents the point of attachment to X, and [symbol] represents the point of attachment to L²;
n is 2;
m is an integer from 1 to 80;
Z is selected from the group consisting of

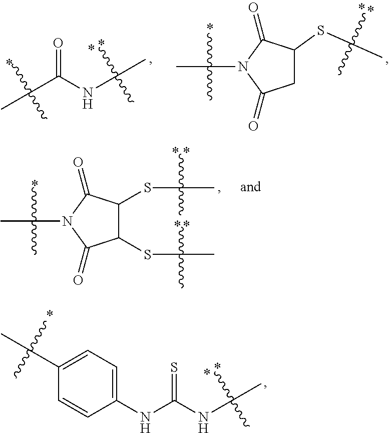

wherein [symbol] represents the point of attachment to L, wherein [symbol] represents the point of attachment to

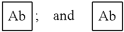

is an antibody.

In another aspect, provided herein are conjugates of the following formula (Va):

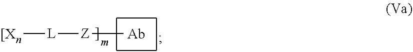

or a pharmaceutically acceptable salt thereof, wherein:

X is a moiety that binds to a cell surface M6PR (e.g., as described herein) or a moiety that binds to a cell surface ASGPR (e.g., as described herein);

L is a linker of the following formula (IIc):

-(L¹)ₐ-(L²)_{b}-(L³)_{c}-(L⁴)_{d}- (IIc); and

wherein
L¹ is

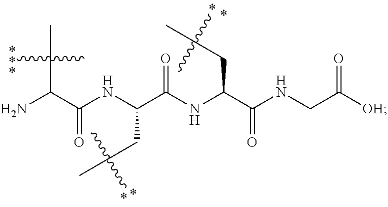 [SMILES: *C[C@H](NC(=O)C(*)N)C(=O)N[C@@H](C*)C(=O)NCC(=O)O]

L² is

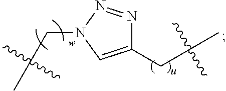

L³ is —NHCO—C₁₋₆-alkylene- or —(OCH₂CH₂)ₚ—;
L⁴ is —NHCO—C₁₋₆-alkylene- or —(OCH₂CH₂)_{q}—;
p and q are each independently an integer of 1 to 20; a is 1, and b, c, and d are each independently 0 or 1; and w and u are each independently an integer of 1 to 10;
wherein [symbol] represents the point of attachment to X, and [symbol] represents the point of attachment to L²;
n is 2;
m is an integer from 1 to 80;
Z is selected from the group consisting of

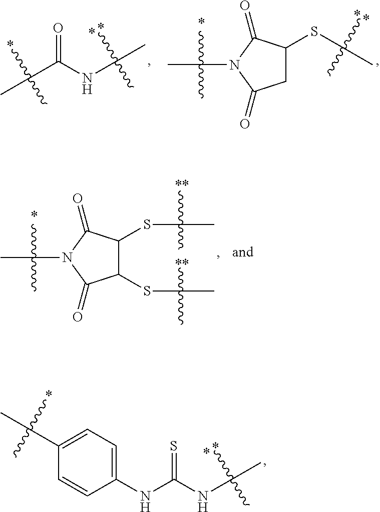

wherein [symbol] represents the point of attachment to L, wherein [symbol] represents the point of attachment to

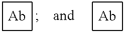

is an antibody.

In another aspect, provided herein are conjugates of the following formula (Va):

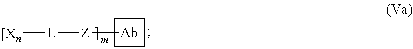

or a pharmaceutically acceptable salt thereof, wherein:

X is a moiety that binds to a cell surface M6PR (e.g., as described herein) or a moiety that binds to a cell surface ASGPR (e.g., as described herein);

L is a linker of the following formula (IId):

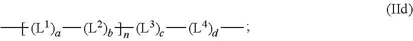

and wherein
L¹ is

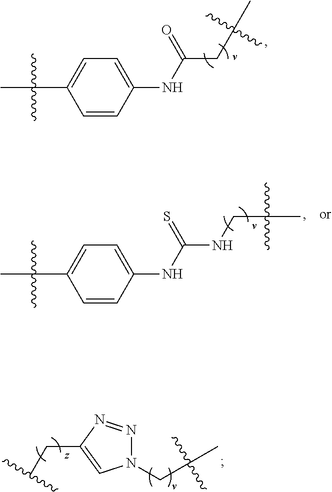

L² is

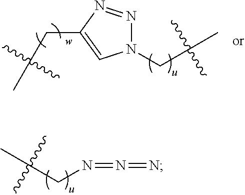

L³ is

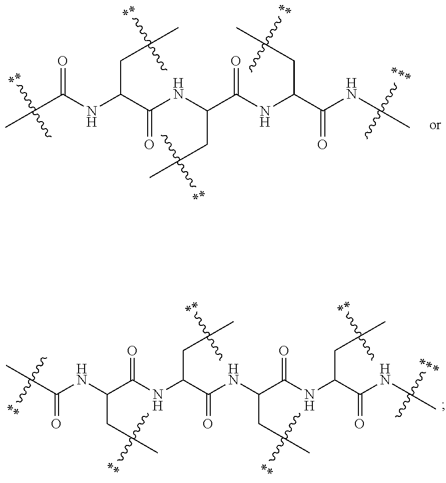 [SMILES: *CC(NC(*)=O)C(=O)NC(C*)C(=O)NC(C*)C(=O)N* ; *CC(NC(*)=O)C(=O)NC(C*)C(=O)NC(C*)C(=O)NC(C*)C(=O)N*]

L⁴ is —CH₂CH₂(OCH₂CH₂)_{q}—;
p is an integer of 1 to 20; c is 1, and a, b, and d are each independently 0 or 1; and u, v, w, and z are each independently an integer of 1 to 10;
wherein [symbol] represents the point of attachment to an H or L², and [symbol] represents the point of attachment to L⁴;
n is an integer of 1 to 5;
m is an integer from 1 to 80;
Z is selected from the group consisting of

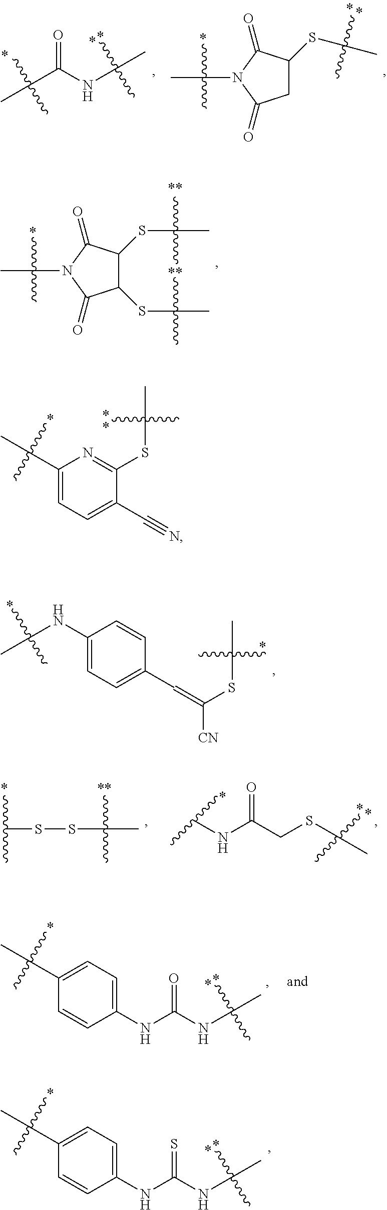

wherein [symbol] represents the point of attachment to L, wherein [symbol] represents the point of attachment to

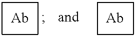

is an antibody.

In another aspect, provided herein are conjugates of the following formula (Vb):

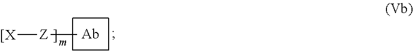

or a pharmaceutically acceptable salt thereof, wherein:
X is a moiety that binds to a cell surface M6PR (e.g., as described herein) or a moiety that binds to a cell surface ASGPR (e.g., as described herein);
m is an integer from 1 to 80;
Z is a moiety having structure of

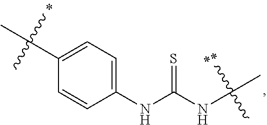

wherein [symbol] represents the point of attachment to X, wherein [symbol] represents the point of attachment to

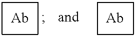

is an antibody.

In certain embodiments of the conjugates of formulas (IVa), (IVb), (Va) and/or (Vb), X is a M6PR binding moiety as described herein, e.g., of formula (XI)-(XVIa), or of of formula (IIIa), (IIIb), (IIIc), or (IIId). In certain embodiments of the conjugates of formulas (IVa), (IVb), (Va) and/or (Vb), X is a M6PR binding moiety as described in any one of the ligands and compounds of Tables 1 and 5-7.

In certain embodiments of the conjugates of formulas (IVa), (IVb), (Va) and/or (Vb), X is a ASGPR binding moiety as described herein, e.g., of formula (IIIa′), (IIIa″), (IIIb′), (IIIb″), (IIIc′), (IIIc″), (IIId′) or (IIId″). In certain embodiments of the conjugates of formulas (IVa), (IVb), (Va) and/or (Vb), X is a ASGPR binding moiety as described in any one of the compounds of Tables 8-9. In certain embodiments of the conjugates of formulas (IVa), (IVb), (Va) and/or (Vb), X is of formula (IIIa″), (IIIb′), or (IIIb″). In certain embodiments of the conjugates of formulas (IVa), (IVb), (Va) and/or (Vb), X is of formula (IIIc′), (IIIc″), (IIId′) or (IIId″). In certain embodiments of the conjugates of formulas (IVa), (IVb), (Va) and/or (Vb), X is of formula (IIIa′) or (IIIa″). In certain embodiments of the conjugates of formulas (IVa), (IVb), (Va) and/or (Vb), X is of formula (IIIb′) or (IIIb″). In certain embodiments of the conjugates of formulas (IVa), (IVb), (Va) and/or (Vb), X is of formula (IIIc′) or (IIIc″). In certain embodiments of the conjugates of formulas (IVa), (IVb), (Va) and/or (Vb), X is of formula (IIId′) or (IIId″).

In certain embodiments, the conjugate of formulas (IVa), (IVb), (Va) and/or (Vb) is is selected from the group consisting of:

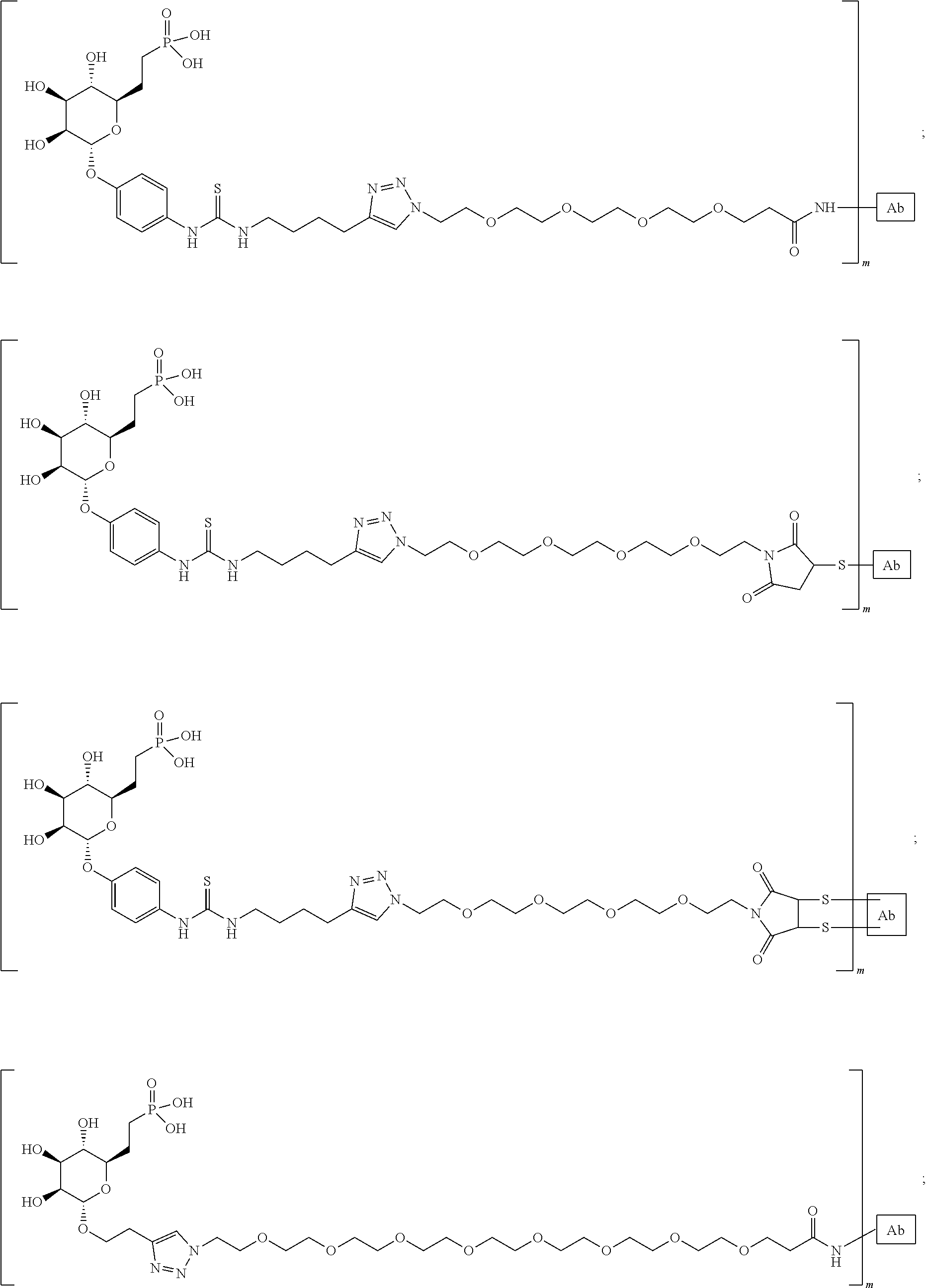

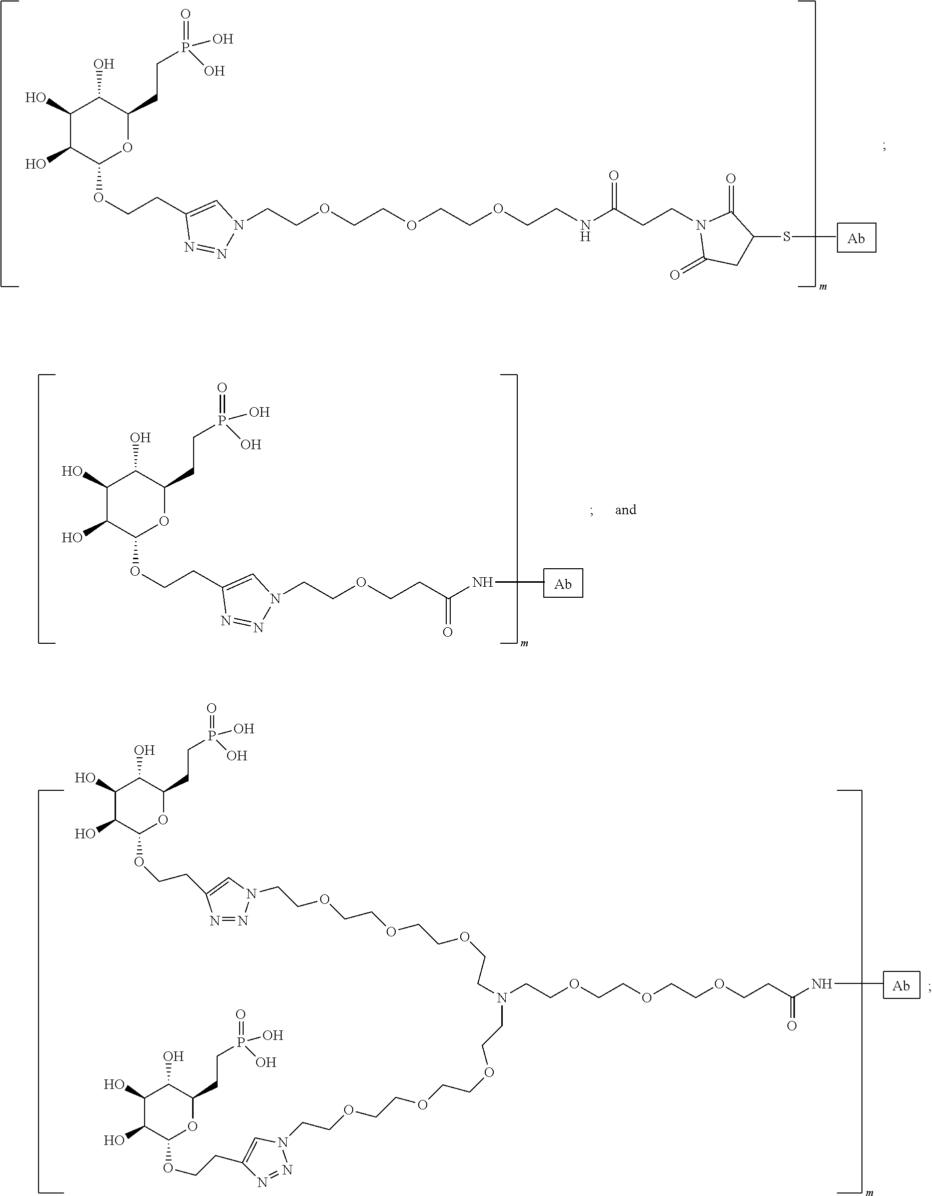

or a pharmaceutically acceptable salt thereof,
wherein:
m is an integer from 1 to 80; and

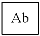

is an antibody.

In certain embodiments, the conjugate of formulas (IVa), (IVb), (Va) and/or (Vb) is is selected from the group consisting of:

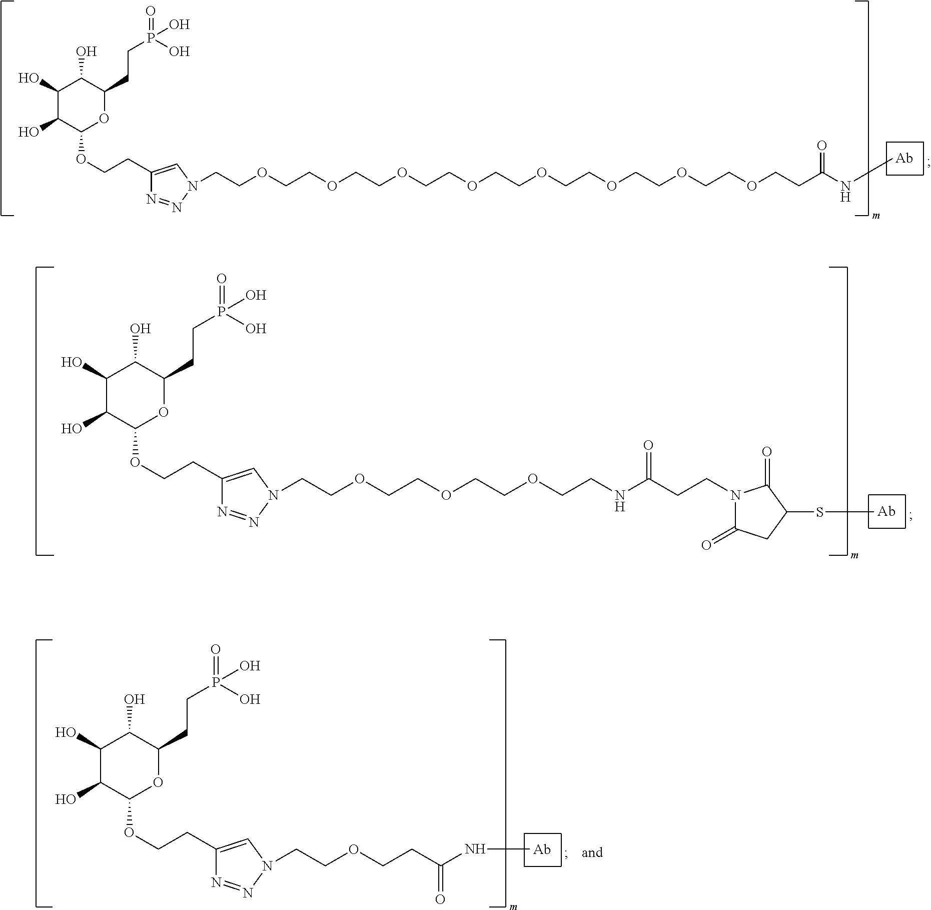

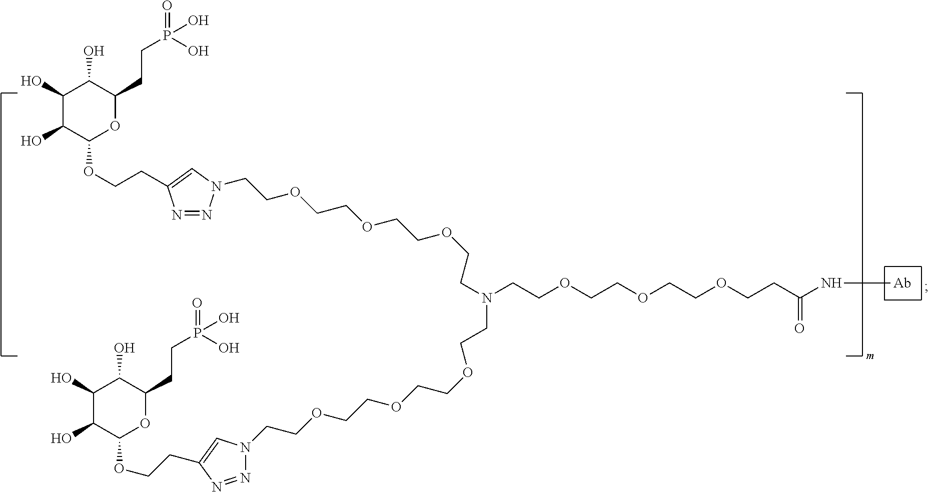

or a pharmaceutically acceptable salt thereof,
wherein:
m is an integer from 1 to 80; and

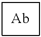

is an antibody.

In certain embodiments, the conjugate of formulas (IVa), (IVb), (Va) and/or (Vb) is is selected from the group consisting of:

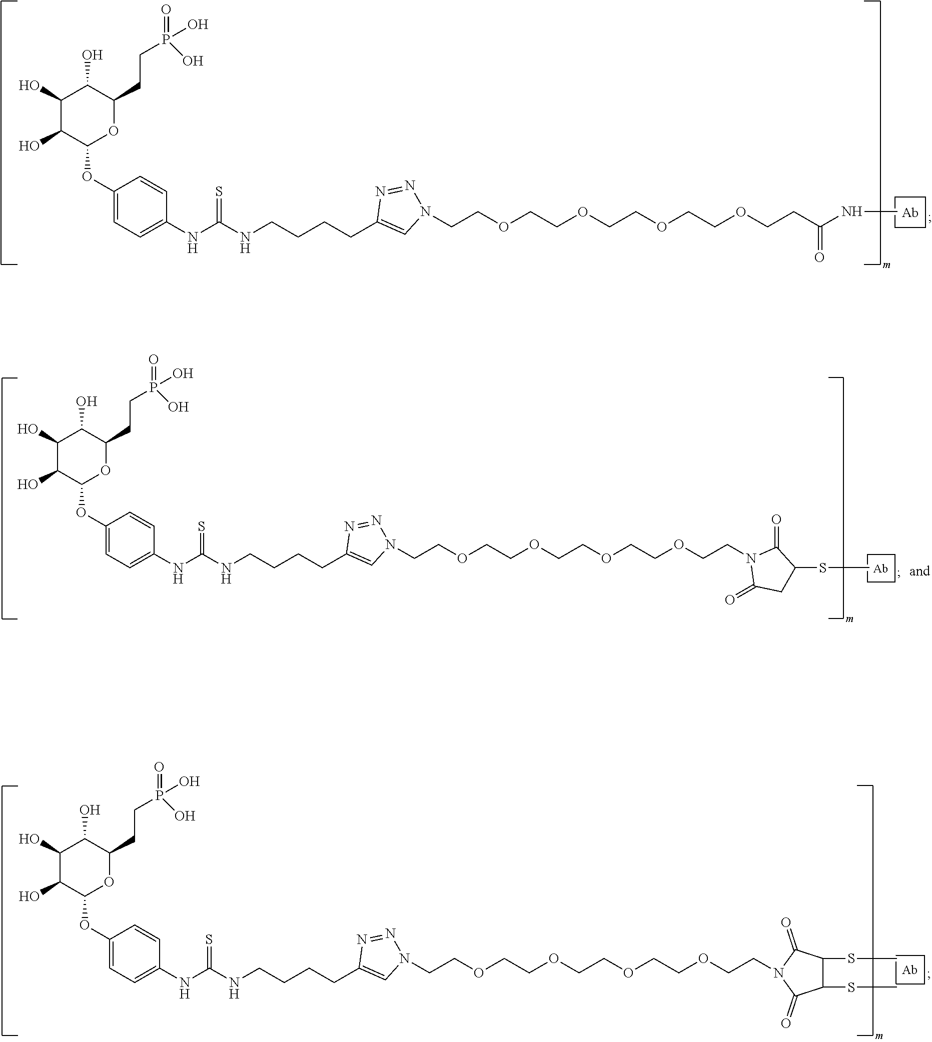

or a pharmaceutically acceptable salt thereof,
wherein:
m is an integer from 1 to 80; and

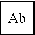

is an antibody.

In certain embodiments, the conjugate of formulas (IVa), (IVb), (Va) and/or (Vb) has the following formula (IX):

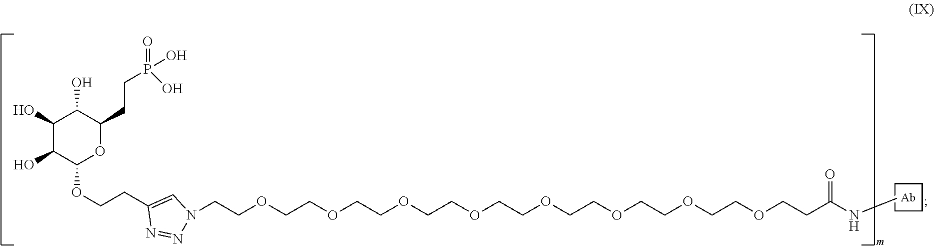

or a pharmaceutically acceptable salt thereof,
wherein:
m is an integer from 1 to 4; and

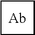

is an antibody.

In certain embodiments, the conjugate of formulas (IVa), (IVb), (Va) and/or (Vb) has the following formula (X):

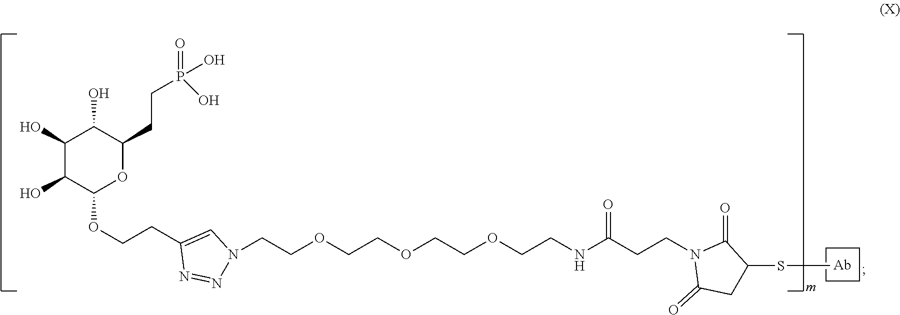

or a pharmaceutically acceptable salt thereof,
wherein:
m is an integer from 1 to 4; and

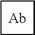

is an antibody.

In certain embodiments, the conjugate of formulas (IVa), (IVb), (Va) and/or (Vb) has the following formula (XI):

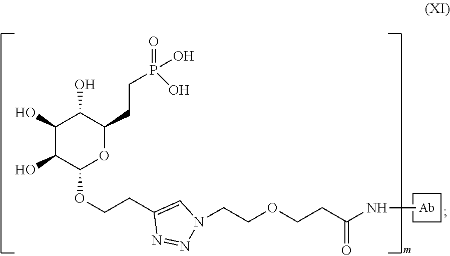

or a pharmaceutically acceptable salt thereof,
wherein:
m is an integer from 1 to 4; and

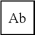

is an antibody.

In certain embodiments, the conjugate of formulas (IVa), (IVb), (Va) and/or (Vb) has the following formula (XII):

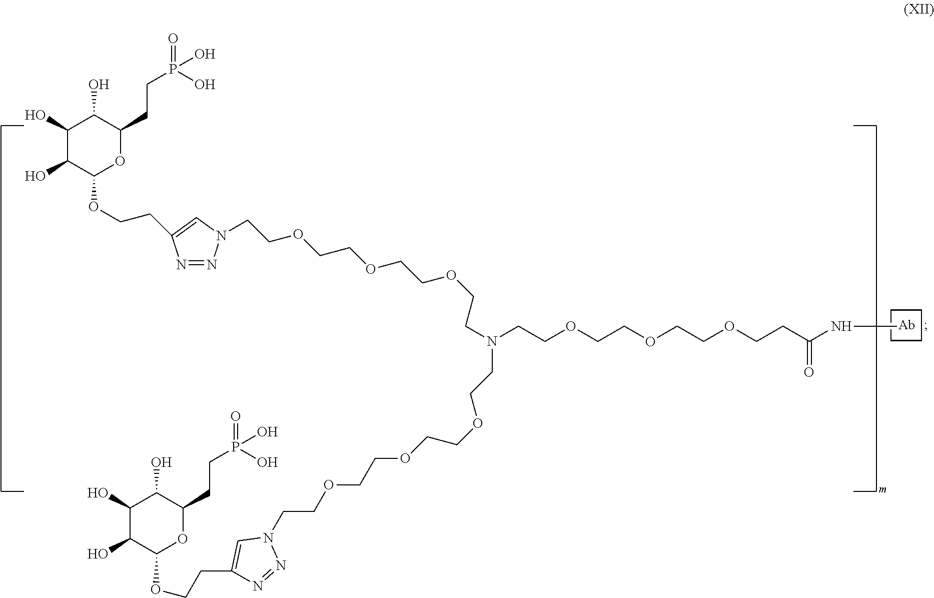

or a pharmaceutically acceptable salt thereof,
wherein:
m is an integer from 1 to 4; and

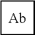

is an antibody.

In another aspect, provided herein are conjugates of the following formula (VIa):

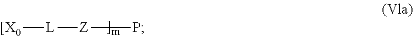

or a pharmaceutically acceptable salt thereof,
wherein:
o is an integer from 1-10;
m is an integer from 1-80;
L is a linker;
Z is selected from the group consisting of

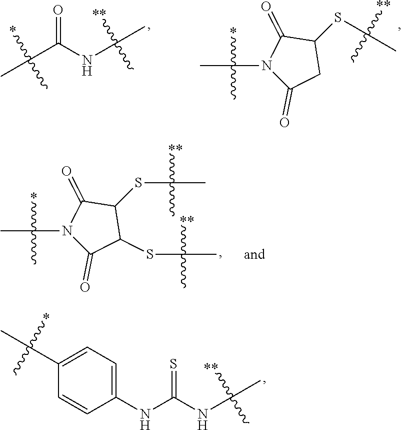

wherein [symbol] represents the point of attachment to L, wherein [symbol] represents the point of attachment to P;
P is a polypeptide;

### X is

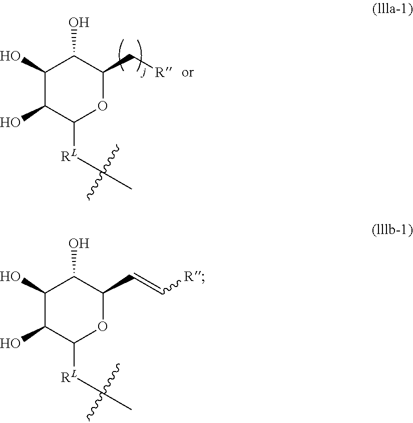

R^{L} is —O—, —NH—, —S— or —CH₂—;
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

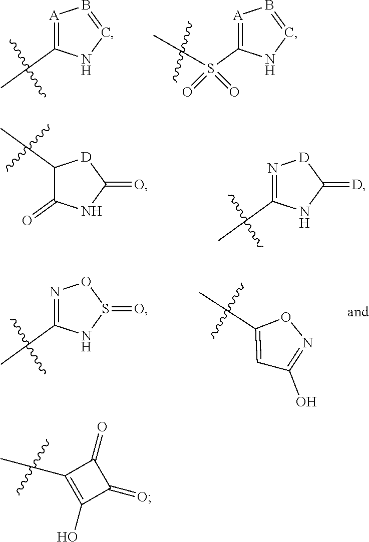

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S; and n, L and Y are as described for formula (Ia); provided
when R^{L} is —O—, R″ is

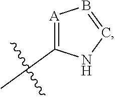

and B and C are N, then j is 2 and provided when R^{L} is and R″ is —CR¹R²COOH, R¹ and R² are not both hydrogen.

In another aspect, provided herein are conjugates of the following formula (VIa):

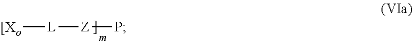

or a pharmaceutically acceptable salt thereof,
wherein:
o is an integer from 1-10;
m is an integer from 1-80;
L is a linker;
Z is selected from the group consisting of

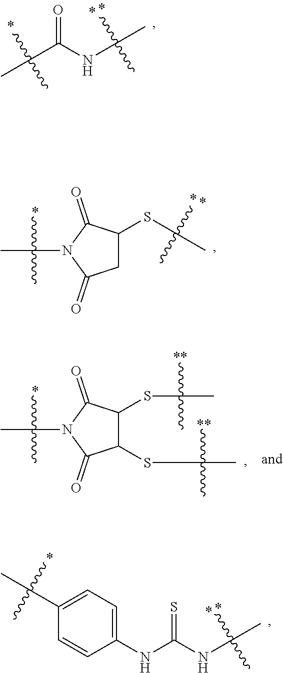

wherein [symbol] represents the point of attachment to L, wherein [symbol] represents the point of attachment to P;
P is a polypeptide;

### X is

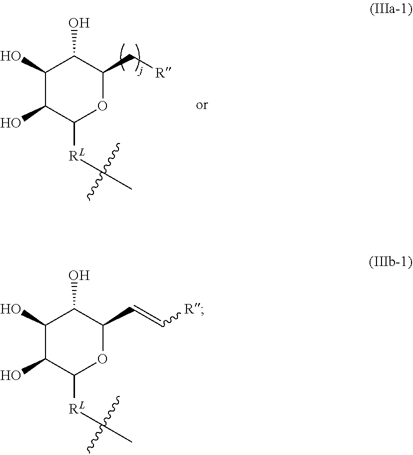

R^{L} is —O—, —NH—, —S— or —CH₂—;
R″ is selected from the group consisting of, —P═O(OH)₂, P(═O)R¹OH, —PH(═O)OH, —(CR¹R²)—P═O(OH)₂, —S(O)OH, —OSO₂OH, —CONH₂, —CONHR³, —CONR³R⁴, —CONH(OH), —CONH(OR³)—CONHSO₂R³, —CONHSO₂NR³R⁴, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

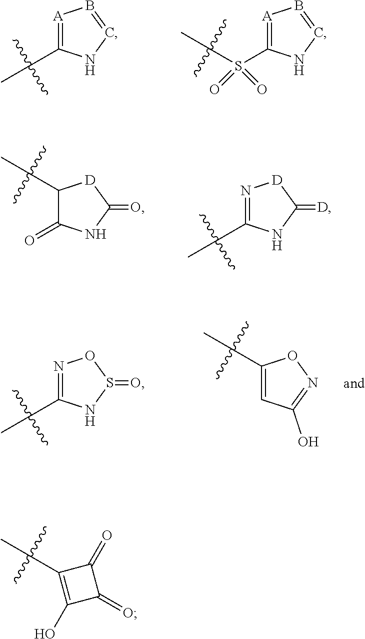

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S; and n, L and Y are as described for formula (Ia); provided when R^{L} is —O—, R″ is

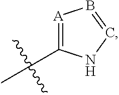

and B and C are N, then j is 2 and provided when R^{L} is —O—, R″ is —CR¹R²COOH, R¹ and R² are not both hydrogen.

In another aspect, provided herein are conjugates of the following formula (VIa):

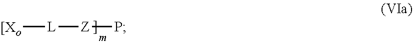

or a pharmaceutically acceptable salt thereof,
wherein:
o is an integer from 1-10;
m is an integer from 1-80;
L is a linker;
Z is selected from the group consisting of

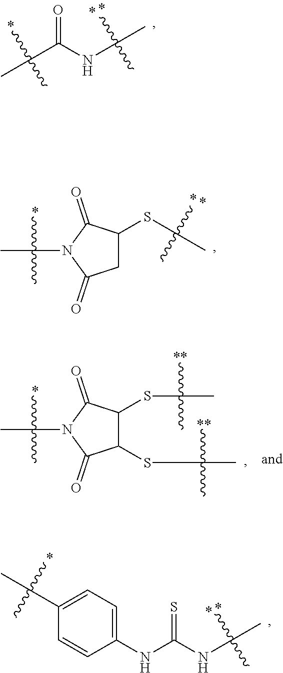

wherein [symbol] represents the point of attachment to L, wherein [symbol] represents the point of attachment to P;
P is a polypeptide;

### X is

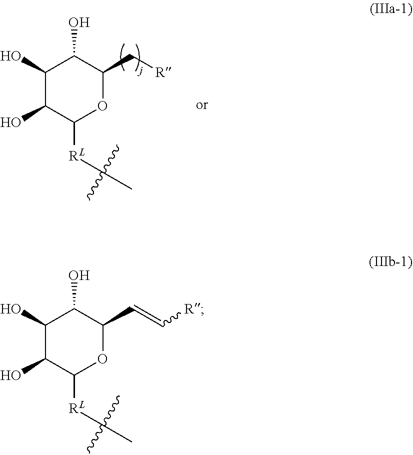

R^{L} is —O—, —NH—, —S— or —CH₂—;
R″ is selected from the group consisting of, —P═O(OH)₂, P(═O)R¹OH, —PH(═O)OH, —(CR¹R²)—P═O(OH)₂, —S(O)OH, —OSO₂OH, —CONH(OH), —CONH(OR³)—CONHSO₂R³, —CONHSO₂NR³R⁴, —SO₂R³, —SOR³R⁴, —SO₂NH₂, —SO₂NHR³, —SO₂NR³R⁴, SO₂NHCOR³, —NHCOR³, —NHC(O)NHS(O)₂R³, —NHSO₂R³,

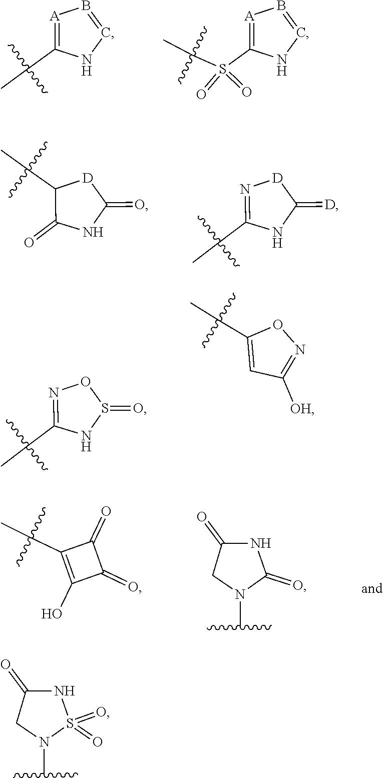

j is an integer of 1 to 3;
R¹ and R² are each independently hydrogen, halo, or CN;
R³ and R⁴ are each independently C₁₋₆ alkyl;
A, B, and C are each independently CH or N; and
D is each independently O or S.

In certain embodiments, the conjugates with their linker structures described herein have weaker binding affinity to cell surface receptors. Without being bound to any particular mechanism or theory, such weaker binding affinity may be corrected to longer half life of the conjugates, and may be useful for tuning (e.g., modifying) the pharmacokinetic properties of the conjugates described herein. In certain embodiments, such weaker binding conjugates still have sufficiently robust uptake.

The term “pharmaceutically acceptable” means being approved by a regulatory agency of the Federal or a state government, or listed in the U.S. Pharmacopeia, European Pharmacopeia or other generally recognized Pharmacopeia for use in animals, and, more particularly in humans.

The term “pharmaceutically acceptable salt” refers to those salts which are suitable for use in contact with the tissues of humans and lower animals without undue toxicity, irritation, allergic response and the like. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the conjugate compounds, or separately by reacting the free base function or group of a compound with a suitable organic acid. Examples of pharmaceutically acceptable salts include, but are not limited to, nontoxic acid addition salts, or salts of an amino group formed with inorganic acids such as hydrochloric acid, hydrobromic acid, phosphoric acid, etc., or with organic acids such as acetic acid, maleic acid, tartaric acid, citric acid, succinic acid or malonic acid. Other pharmaceutically acceptable salts include, but are not limited to, adipate, alginate, ascorbate, benzenesulfonate, benzoate, bisulfate, citrate, digluconate, dodecylsulfate, ethanesulfonate, formate, fumarate, gluconate, 2-hydroxy-ethanesulfonate, lactate, laurate, malate, maleate, malonate, methanesulfonate, oleate, oxalate, palmitate, phosphate, propionate, stearate, succinate, sulfate, tartrate, p-toluenesulfonate, valerate salts, and the like. Representative alkali or alkaline earth metal salts include sodium, lithium, potassium, calcium, or magnesium salts, and the like. Further pharmaceutically acceptable salts include, nontoxic ammonium, quaternary ammonium, and amine cations formed using counterions such as halide, hydroxide, carboxylate, sulfate, phosphate, nitrate, alkyl groups having from 1 to 6 carbon atoms (e.g., C₁₋₆ alkyl), sulfonate and aryl sulfonate.

Conjugates of the polypeptide (P), e.g., an antibody (Ab) and compound (Xn-L-Y) may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In certain embodiments of the conjugates described herein, L is bonded through an amide bond to a lysine residue of P. In certain embodiments of the conjugates described herein, L is bonded through a thioether bond to a cysteine residue of P. In certain embodiments of the conjugates described herein, L is bonded through an amide bond to a lysine residue of Ab, as depicted above. In certain embodiments of the conjugates described herein, L is bonded through a thioether bond to a cysteine residue of Ab, as depicted above. In certain embodiments of the conjugates described herein, L is bonded through two thioether bonds to two cysteine residues of Ab, wherein the two cysteine residues are from an opened cysteine-cysteine disulfide bond in Ab, as depicted above. In certain embodiments, the opened cysteine-cysteine disulfide bond is an interchain disulfide bond.

In certain embodiments of the conjugates described herein, when L is bonded through an amide bond to a lysine residue of P, m is an integer from 1 to 80. In certain embodiments of the conjugates described herein, when L is bonded through a thioether bond to a cysteine residue of P, m is an integer from 1 to 8.

In certain embodiments, conjugation to the polypeptide P or the antibody Ab may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide or antibody folding and assembly or alter polypeptide or antigen binding (see, e.g., Junutula et al., J. Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). Yet other non-limiting techniques that allow for site-specific conjugation to polypeptides or antibodies include engineering of non-natural amino acids, including, e.g., p-acetylphenylalanine (p-acetyl-Phe), p-azidomethyl-N-phenylalanine (p-azidomethyl-Phe), and azidolysine (azido-Lys) at specific linkage sites, and can further include engineering unique functional tags, including, e.g., LPXTG, LLQGA, sialic acid, and GlcNac, for enzyme mediated conjugation. See Jackson, Org. Process Res. Dev. 2016; 20: 852-866; and Tsuchikama and An, Protein Cell 2018; 9(1):33-46, the contents of each of which is incorporated by reference in its entirety. See also US 2019/0060481 A1 & US 2016/0060354 A1, the contents of each of which is incorporated by reference in its entirety All such methodologies are contemplated for use in connection with making the conjugates described herein.

Loading of the compounds of formulas (Ia) and (Ib) to the polypeptides (e.g., antibodies) described herein is represented by “m” in formulas (IVa), (IVb), (Va) and/or (Vb), and is the average number of units of “Xn-L-” or “Xn-” per conjugate molecule. As used herein, the term “DAR” refers to the average value of “m” or the loading of the conjugate. The number of “X” moieties (e.g., M6P moieties) per each unit of “Xn-L-” or “Xn-” is represented by “n” in formulas (IVa), (IVb), (Va) and/or (Vb). As used herein, the term “valency” or “valencies” refers to the number of “X” moieties per unit (“n”). It will be understood that loading, or DAR, is not necessarily equivalent to the number of “X” moieties per conjugate molecule. By means of example, where there is one “X” moiety per unit (n=1; valency is “1”), and one “Xn-L-” unit per conjugate (m=1), there will be 1×1=1 “X” moiety per conjugate. However, where there are two “X” moieties per unit (n=2; valency is “2”), and four “Xn-L-” units per conjugate (m=4), there will be 2×4=8 “X” moieties per conjugate. Accordingly, for the conjugates described herein, the total number of “X” moieties per conjugate molecule will be n x m. As used herein, the term “total valency” or “total valencies” refers to the total number of “X” moieties per conjugate molecule (n x m; total valency).

DAR (loading) may range from 1 to 80 units per conjugate. The conjugates provided herein may include collections of polypeptides, antibodies or antigen binding fragments conjugated with a range of units, e.g., from 1 to 80. The average number of units per polypeptide or antibody in preparations of the conjugate from conjugation reactions may be characterized by conventional means such as mass spectroscopy. The quantitative distribution of DAR (loading) in terms of m may also be determined. In some instances, separation, purification, and characterization of homogeneous conjugate where m is a certain value may be achieved by means such as electrophoresis.

In certain embodiments, the DAR for a conjugate provided herein ranges from 1 to 80. In certain embodiments, the DAR for a conjugate provided herein ranges from 1 to 70. In certain embodiments, the DAR for a conjugate provided herein ranges from 1 to 60. In certain embodiments, the DAR for a conjugate provided herein ranges from 1 to 50. In certain embodiments, the DAR for a conjugate provided herein ranges from 1 to 40. In certain embodiments, the DAR for a conjugate provided herein ranges from 1 to 35. In certain embodiments, the DAR for a conjugate provided herein ranges from 1 to 30. In certain embodiments, the DAR for a conjugate provided herein ranges from 1 to 25. In certain embodiments, the DAR for a conjugate provided herein ranges from 1 to 20. In certain embodiments, the DAR for a conjugate provided herein ranges from 1 to 18. In certain embodiments, the DAR for a conjugate provided herein ranges from 1 to 15. In certain embodiments, the DAR for a conjugate provided herein ranges from 1 to 12. In certain embodiments, the DAR for a conjugate provided herein ranges from 1 to 10. In certain embodiments, the DAR for a conjugate provided herein ranges from 1 to 9. In certain embodiments, the DAR for a conjugate provided herein ranges from 1 to 8. In certain embodiments, the DAR for a conjugate provided herein ranges from 1 to 7. In certain embodiments, the DAR for a conjugate provided herein ranges from 1 to 6. In certain embodiments, the DAR for a conjugate provided herein ranges from 1 to 5. In certain embodiments, the DAR for a conjugate provided herein ranges from 1 to 4. In certain embodiments, the DAR for a conjugate provided herein ranges from 1 to 3. In certain embodiments, the DAR for a conjugate provided herein ranges from 2 to 12. In certain embodiments, the DAR for a conjugate provided herein ranges from 2 to 10. In certain embodiments, the DAR for a conjugate provided herein ranges from 2 to 9. In certain embodiments, the DAR for a conjugate provided herein ranges from 2 to 8. In certain embodiments, the DAR for a conjugate provided herein ranges from 2 to 7. In certain embodiments, the DAR for a conjugate provided herein ranges from 2 to 6. In certain embodiments, the DAR for a conjugate provided herein ranges from 2 to 5. In certain embodiments, the DAR for a conjugate provided herein ranges from 2 to 4. In certain embodiments, the DAR for a conjugate provided herein ranges from 3 to 12. In certain embodiments, the DAR for a conjugate provided herein ranges from 3 to 10. In certain embodiments, the DAR for a conjugate provided herein ranges from 3 to 9. In certain embodiments, the DAR for a conjugate provided herein ranges from 3 to 8. In certain embodiments, the DAR for a conjugate provided herein ranges from 3 to 7. In certain embodiments, the DAR for a conjugate provided herein ranges from 3 to 6. In certain embodiments, the DAR for a conjugate provided herein ranges from 3 to 5. In certain embodiments, the DAR for a conjugate provided herein ranges from 3 to 4.

In certain embodiments, the DAR for a conjugate provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the DAR for a conjugate provided herein is about 1, about 2, about 3, about 4, about 5, about 6, about 7, about 8, about 9, about 10, about 11, about 12, or more. In some embodiments, the DAR for a conjugate provided herein is about 3.1, about 3.2, about 3.3, about 3.4, about 3.5, about 3.6, about 3.7, about 3.8, or about 3.9.

In some embodiments, the DAR for a conjugate provided herein ranges from 2 to 20, 2 to 19, 2 to 18, 2 to 17, 2 to 16, 2 to 15, 2 to 14, or 2 to 13. In some embodiments, the DAR for a conjugate provided herein ranges from 3 to 20, 3 to 19, 3 to 18, 3 to 17, 3 to 16, 3 to 15, 3 to 14, or 3 to 13. In some embodiments, the DAR for a conjugate provided herein is about 1. In some embodiments, the DAR for a conjugate provided herein is about 2. In some embodiments, the DAR for a conjugate provided herein is about 3. In some embodiments, the DAR for a conjugate provided herein is about 4. In some embodiments, the DAR for a conjugate provided herein is about 3.8. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8. In some embodiments, the DAR for a conjugate provided herein is about 9. In some embodiments, the DAR for a conjugate provided herein is about 10. In some embodiments, the DAR for a conjugate provided herein is about 11. In some embodiments, the DAR for a conjugate provided herein is about 12. In some embodiments, the DAR for a conjugate provided herein is about 13. In some embodiments, the DAR for a conjugate provided herein is about 14. In some embodiments, the DAR for a conjugate provided herein is about 15. In some embodiments, the DAR for a conjugate provided herein is about 16. In some embodiments, the DAR for a conjugate provided herein is about 17. In some embodiments, the DAR for a conjugate provided herein is about 18. In some embodiments, the DAR for a conjugate provided herein is about 19. In some embodiments, the DAR for a conjugate provided herein is about 20.

In some embodiments, the DAR for a conjugate provided herein is about 25. In some embodiments, the DAR for a conjugate provided herein is about 30. In some embodiments, the DAR for a conjugate provided herein is about 35. In some embodiments, the DAR for a conjugate provided herein is about 40. In some embodiments, the DAR for a conjugate provided herein is about 50. In some embodiments, the DAR for a conjugate provided herein is about 60. In some embodiments, the DAR for a conjugate provided herein is about 70. In some embodiments, the DAR for a conjugate provided herein is about 80.

In certain embodiments, fewer than the theoretical maximum of units are conjugated to the polypeptide, e.g., antibody, during a conjugation reaction. A polypeptide may contain, for example, lysine residues that do not react with the compound or linker reagent. Generally, for example, antibodies do not contain many free and reactive cysteine thiol groups which may be linked to a drug unit; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the compound is conjugated via a lysine residue on the antibody. In some embodiments, the linker unit or a drug unit is conjugated via a cysteine residue on the antibody.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

The DAR (loading) of a conjugate may be controlled in different ways, e.g., by: (i) limiting the molar excess of compound or conjugation reagent relative to polypeptide, (ii) limiting the conjugation reaction time or temperature, (iii) partial or limiting reductive conditions for cysteine thiol modification, (iv) engineering by recombinant techniques the amino acid sequence of the polypeptide, such that the number and position of cysteine residues is modified for control of the number and/or position of linker-drug attachments (such as for thiomabs prepared as disclosed in WO2006/034488 (herein incorporated by reference in its entirety)).

It is to be understood that the preparation of the conjugates described herein may result in a mixture of conjugates with a distribution of one or more units attached to a polypeptide, for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

### Polypeptides (P):

In certain embodiments, the polypeptide (P) of the conjugate comprises a polypeptide that binds to a soluble (e.g., secreted) polypeptide of interest. In certain embodiments, for example, the polypeptide of interest is a ligand that binds a cell surface receptor and P comprises the ligand binding portion of the cell surface receptor, for example, the extracellular domain of the cell surface receptor, e.g., a ligand-binding domain of the extracellular domain of the cell surface receptor. In certain embodiments, polypeptide of interest is a cell surface receptor and P comprises a ligand that binds the cell surface receptor or a receptor-binding portion of the ligand.

A polypeptide (P) that binds to a polypeptide of interest binds as “binding” in this context is understood by one skilled in the art. For example, P, e.g., an antibody, or a conjugate as described herein comprising such P, may bind to other polypeptides, generally with lower affinity as determined by, e.g., immunoassays or other assays known in the art. In a specific embodiment, P, or a conjugate as described herein comprising such P that specifically bind to a polypeptide of interest binds to the polypeptide of interest with an affinity that is at least 2 logs, 2.5 logs, 3 logs, 4 logs or greater than the affinity when P or the conjugate bind to another polypeptide. In another specific embodiment, P, or a conjugate as described herein comprising such P, does not specifically bind a polypeptide other than the polypeptide of interest. In a specific embodiment, P, or a conjugate as described herein comprising P, specifically binds to a polypeptide of interest with an affinity (K_{d}) less than or equal to 20 mM. In particular embodiments, such binding is with an affinity (K_{d}) less than or equal to about 20 mM, about 10 mM, about 1 mM, about 100 uM, about 10 uM, about 1 uM, about 100 nM, about 10 nM, or about 1 nM. Unless otherwise noted, “binds,” “binds to,” “specifically binds” or “specifically binds to” in this context are used interchangeably.

In certain embodiments, for example, the polypeptide of interest is a cell surface receptor and P comprises an antibody that binds to the cell surface protein, e.g., the extracellular domain of the cell surface receptor. In other embodiments, for example, the polypeptide of interest is a soluble, (e.g., secreted) polypeptide of interest, for example the ligand for a cell surface receptor polypeptide, and P comprises an antibody that binds to the ligand.

Polypeptides may contain L-amino acids, D-amino acids, or both and may contain any of a variety of amino acid modifications or analogs known in the art. Useful modifications include, e.g., terminal acetylation, amidation, methylation, etc.

In certain embodiments, the polypeptide (P) comprises about 10, about 20, about 30, about 40, about 50, about 100, about 150, about 200, about 250, about 300, about 350, about 400, about 450, about 500, about 550, about 600, about 650, about 700, about 750, about 800, about 850, about 900, or about 950 amino acids.

In certain embodiments, the polypeptide (P) comprises about 10-50, about 50-100, about 100-150, about 150-200, about 200-250, about 250-300, about 300-350, about 350-400, about 400-450, about 450-500, about 500-600, about 600-700, about 700-800, about 800-900, or about 900-1000 amino acids.

In certain embodiments, the conjugate comprises an antibody, Ab. In certain embodiments, Ab is a monoclonal antibody. In certain embodiments, Ab is a human antibody. In certain embodiments, Ab is a humanized antibody. In certain embodiments, Ab is a chimeric antibody. In certain embodiments, Ab is a full-length antibody that comprises two heavy chains and two light chains. In particular embodiments, Ab is an IgG antibody, e.g., is an IgG1, IgG2, IgG3 or IgG4 antibody. In certain embodiments, Ab is a single chain antibody. In yet other embodiments, Ab is an antigen-binding fragment of an antibody, e.g., a Fab fragment.

In certain embodiments, the antibody specifically binds to a cancer antigen.

In certain embodiments, the antibody specifically binds to a hepatocyte antigen.

In certain embodiments, the antibody specifically binds to an antigen presented on a macrophage.

In certain embodiments, the antibody specifically binds to an intact complement or a fragment thereof. In certain embodiments, the antibody specifically binds to one or more immunodominant epitope(s) within intact complement or a fragment thereof.

In certain embodiments, the antibody specifically binds to a cell surface receptor. In certain embodiments, the antibody specifically binds to a cell surface receptor ligand.

In certain embodiments, the antibody specifically binds to an epidermal growth factor (EGF) protein, e.g., a human EGF. In certain embodiments, the antibody specifically binds to one or more immunodominant epitope(s) within an EGF protein.

In certain embodiments, the antibody specifically binds to an epidermal growth factor receptor (EGFR) protein, e.g., a human EGFR. In certain embodiments, the antibody specifically binds to one or more immunodominant epitope(s) within an EGFR protein. In a certain embodiment, the antibody comprises the CDRs present in cetuximab. In another certain embodiment, the antibody comprises the variable light chain and variable heavy chain present in cetuximab. In a particular embodiment, the antibody is cetuximab. In a certain embodiment, the antibody comprises the CDRs present in matuzumab. In another certain embodiment, the antibody comprises the variable light chain and variable heavy chain present in matuzumab. In a particular embodiment, the antibody is matuzumab.

In certain embodiments, the antibody specifically binds to vascular endothelial growth factor (VEGF) protein, e.g., human VEGF protein. In certain embodiments, the antibody specifically binds to one or more immunodominant epitope(s) within a VEGF protein.

In certain embodiments, the antibody specifically binds to a vascular endothelial growth factor receptor (VEGFR) protein, e.g., human VEGFR protein. In particular embodiments, the antibody specifically binds vascular endothelial growth factor receptor 2 (VEGFR2) protein, e.g., a human VEGFR2 protein. In other particular embodiments, the antibody specifically binds a vascular endothelial growth factor receptor 3 (VEGFR3) protein, e.g., a human VEGFR3 protein. In certain embodiments, the antibody specifically binds to one or more immunodominant epitope(s) within a VEGFR protein, a VEGFR2 protein or a VEGFR3 protein.

In certain embodiments, the antibody specifically binds to a fibroblast growth factor (FGF), e.g., a human FGF. In certain embodiments, the antibody specifically binds to one or more immunodominant epitope(s) within a FGF protein.

In certain embodiments, the antibody specifically binds to a fibroblast growth factor receptor (FGFR), e.g., a human FGFR. In particular embodiments, the antibody specifically binds fibroblast growth factor receptor 2 (FGFR2) protein, e.g., a human FGFR2 protein, for example, a FGFR2b protein. In other particular embodiments, the antibody specifically binds a fibroblast growth factor receptor 3 (FGFR3) protein, e.g., a human FGFR3 protein. In certain embodiments, the antibody specifically binds to one or more immunodominant epitope(s) within a FGFR protein, a FGFR2 protein or a FGFR3 protein. In a certain embodiment, the antibody comprises the CDRs present in vofatamab. In another certain embodiment, the antibody comprises the variable light chain and the variable heavy chain present in vofatamab. In a particular embodiment is vofatamab. In a certain embodiment, the antibody comprises the CDRs present in bemarituzumab. In another certain embodiment, the antibody comprises the variable light chain and the variable heavy chain present in bemarituzumab. In a particular embodiment is bemarituzumab.

In certain embodiments, the antibody specifically binds to a receptor tyrosine kinase cMET protein. In certain embodiments, the antibody specifically binds to one or more immunodominant epitope(s) within a receptor tyrosine kinase cMET protein. In certain embodiments, the antibody comprises the CDRs present in onartuzumab (MetMAb; see, e.g., CAS number 1133766-06-9). In certain embodiments, the antibody comprises the variable light chain and the heavy chain present in onartuzumab. In certain embodiments, the antibody is onartuzumab. In certain embodiments, the antibody comprises the CDRs present in emibetuzumab (LY2875358; see, e.g., CAS number 1365287-97-3). In certain embodiments, the antibody comprises the variable light chain and the heavy chain present in emibetuzumab. In certain embodiments, the antibody is emibetuzumab. In certain embodiments, the antibody specifically binds to a CD47 protein, e.g., a human CD47 protein. In certain embodiments, the antibody specifically binds to one or more immunodominant epitope(s) within a CD47 protein. In a certain embodiment, the antibody comprises the CDRs present in Hu5F9-G4 (5F9). In another certain embodiment, the antibody comprises the variable light chain and the variable heavy chain present in Hu5F9-G4 (5F9). In a particular embodiment is Hu5F9-G4 (5F9).

In certain embodiments, the antibody specifically binds to an immune checkpoint inhibitor. In certain embodiments, the antibody binds to one or more immunodominant epitope(s) within an immune checkpoint inhibitor.

In certain embodiments, the antibody specifically binds to a programmed death protein, e.g., a human PD-1. In certain embodiments, the antibody specifically binds to one or more immunodominant epitope(s) within PD-1 protein. In a certain embodiment, the antibody comprises the CDRs present in nivolumab. In another certain embodiment, the antibody comprises the variable light chain and variable heavy chain present in nivolumab. In a particular embodiment, the antibody is nivoumab. In a certain embodiment, the antibody comprises the CDRs present in pembrolizumab. In another certain embodiment, the antibody comprises the variable light chain and variable heavy chain present in pembrolizumab. In a particular embodiment, the antibody is pembrolizumab.

In certain embodiments, the antibody specifically binds to a programmed death ligand-1 (PD-L1) protein, e.g., a human PD-L1. In certain embodiments, the antibody specifically binds to one or more immunodominant epitope(s) within PD-L1 protein. In a certain embodiment, the antibody comprises the CDRs present in atezolizumab. In another certain embodiment, the antibody comprises the variable light chain and variable heavy chain present in atezolizumab. In a particular embodiment, the antibody is atezolizumab. In a certain embodiment, the antibody comprises the CDRs present in 29E.2A3 (BioXCell). In another certain embodiment, the antibody comprises the variable light chain and variable heavy chain present in 29E.2A3. In a particular embodiment, the antibody is 29E.2A3.

In certain embodiments, the antibody binds to TIM3. In certain embodiments, the antibody binds to one or more immunodominant epitope(s) within TIM3.

In certain embodiments, the antibody specifically binds to a lectin. In certain embodiments, the antibody specifically binds to one or more immunodominant epitope(s) within a lectin. In certain embodiments, the antibody binds to SIGLEC. In certain embodiments, the antibody binds to one or more immunodominant epitope(s) within SIGLEC. In certain embodiments, the antibody binds to a cytokine receptor. In certain embodiments, the antibody binds to a one or more immunodominant epitope(s) within cytokine receptor. In certain embodiments, the antibody binds to sIL6R. In certain embodiments, the antibody binds to one or more immunodominant epitope(s) within sIL6R. In certain embodiments, the antibody binds to a cytokine. In certain embodiments, the antibody binds to one or more immunodominant epitope(s) within a cytokine. In yet certain embodiments, the antibody binds to MCP-1, TNF (e.g., a TNFalpha), IL1a, IL1b, IL4, IL5, IL6, IL12/IL23, IL13, IL17 or p40. In yet certain embodiments, the antibody binds to one or more immunodominant epitope(s) within MCP-1, TNF (e.g., a TNFalpha), IL1a, IL1b, IL4, IL5, IL6, IL12/IL23, IL13, IL17 or p40.

In certain embodiments, the antibody binds to a major histocompatibility protein (e.g., a MHC class I or class II molecule). In certain embodiments, the antibody binds to one or more immunodominant epitope(s) within a major histocompatibility protein (e.g., a MHC class I or class II molecule). In certain embodiments, the antibody binds to beta 2 microglobulin. In certain embodiments, the antibody binds to one or more immunodominant epitope(s) within beta 2 microglobulin.

The heavy chain and light chain sequences of an exemplary anti-EGFR antibody (see, e.g., cetuximab, CAS number 205923-56-4) are shown in Table A.

TABLE A
Heavy chain
QVQLKQSGPGLVQPSQSLSITCTVSGFSLTNYGVHWVRQSPGKGL
EWLGVIWSGGNTDYNTPFTSRLSINKDNSKSQVFFKMNSLQSNDT
AIYYCARALTYYDYEFAYWGQGTLVTVSAASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSRDEL
TKNQVSLTCLVKGFYPSDIAVEWESNGQPENNYKTTPPVLDSDGS
FFLYSKLTVDKSRWQQGNVFSCSVMHEALHNHYTQKSLSLSPGK
(SEQ ID NO: 1)
Light chain
DILLTQSPVILSVSPGERVSFSCRASQSIGTNIHWYQQRTNGSPR
LLIKYASESISGIPSRFSGSGSGTDFTLSINSVESEDIADYYCQQ
NNNWPTTFGAGTKLELKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
(SEQ ID NO: 2)

The heavy chain and light chain sequences of an exemplary Fab fragment of an anti-EGFR antibody (see, e.g., matuzumab, NCBI Accession Nos. 3C09H_H and 3C09_L, CAS number 339186-68-4) are shown in Table B.

TABLE B
Heavy chain Fab
QVQLVQSGAEVKKPGASVKVSCKASGYTFTSHWMHWVRQAPGQGL
EWIGEFNPSNGRTNYNEKFKSKATMTVDTSTNTAYMELSSLRSED
TAVYYCASRDYDYAGRYFDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKS
(SEQ ID NO: 3)
Light chain
DIQMTQSPSSLSASVGDRVTITCSASSSVTYMYWYQQKPGKAPKL
LIYDTSNLASGVPSRFSGSGSGTDYTFTISSLQPEDIATYYCQQW
SSHIFTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGE
(SEQ ID NO: 4)

The heavy chain and light chain sequences of an exemplary anti-PD-L1 antibody (see, e.g., atezolizumab, CAS number 138723-44-3) are shown in Table C.

TABLE C
Heavy chain
EVQLVESGGGLVQPGGSLRLSCAASGFTFSDSWIHWRQAPGKGLE
WVAWISPYGGSTYYADSVKGRFTISADTSKNTAYLQMNSLRAEDT
AVYYCARRHWPGGFDYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYASTYRVVSVLTVLHQDWLN
GKEYKCKVSNKALPAPIEKTISKAKGQPREPQVYTLPPSREEMTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 5)
Light chain
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
(SEQ ID NO: 6)

### Pharmaceutical Compositions

In another embodiment, provided herein are pharmaceutical compositions comprising one or more conjugates disclosed herein and a pharmaceutically acceptable carrier.

In certain embodiments, the pharmaceutical compositions provided herein contain therapeutically effective amounts of one or more of the conjugates provided herein, and optionally one or more additional prophylactic or therapeutic agents, in a pharmaceutically acceptable carrier. Pharmaceutical compositions may be useful for the prevention, treatment, management or amelioration of a disease or disorder described herein or one or more symptoms thereof.

Pharmaceutical carriers suitable for administration of the conjugates provided herein include any such carriers known to those skilled in the art to be suitable for the particular mode of administration.

The conjugates described herein can be formulated as the sole pharmaceutically active ingredient in the composition or can be combined with other active ingredients.

In certain embodiments, the conjugate is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, a conjugate described herein may be mixed with a suitable pharmaceutical carrier. The concentration of the conjugate in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of conjugate is dissolved, suspended, dispersed or otherwise mixed in a selected carrier at an effective concentration such that the treated condition is relieved, prevented, or one or more symptoms are ameliorated.

Concentrations of the conjugate in a pharmaceutical composition provided herein will depend on, e.g., the physicochemical characteristics of the conjugate, the dosage schedule, and amount administered as well as other factors known to those of skill in the art.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a conjugate sufficient to produce the desired therapeutic effect, in association with the required pharmaceutical carrier, vehicle or diluent. Examples of unit-dose forms include ampoules and syringes and individually packaged capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the conjugates herein are in a liquid pharmaceutical formulation. Liquid pharmaceutically administrable formulations can, for example, be prepared by dissolving, dispersing, or otherwise mixing a conjugate and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (2012) 22nd ed., Pharmaceutical Press, Philadelphia, Pa. Dosage forms or compositions containing antibody in the range of 0.005% to 100% with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a conjugate described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a conjugate provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the conjugates provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

### Uses and Methods:

In one aspect, provided herein are methods of using the conjugates described herein to remove a polypeptide of interest (a target protein) from a cell's surface. In one aspect, provided herein are methods of using the conjugates described herein to remove a polypeptide of interest (a target protein) from the extracellular milieu. For example, in one embodiment, provided herein are methods of using the conjugates described herein to remove a polypeptide of interest (a target protein) from the surface of a cell by sequestering the target protein in the cell's lysosome. In another embodiment, provided herein are methods of using the conjugates described herein to remove a polypeptide of interest (a target protein) from the extracellular space (the extracellular milieu) of a cell by sequestering the target protein in the cell's lysosome. In another embodiment, provided herein are methods of using the conjugates described herein to remove a polypeptide of interest (a target protein) from the surface of a cell by sequestering the target protein in the cell's lysosome and degrading the target protein. In another embodiment, provided herein are methods of using the conjugates described herein to remove a polypeptide of interest (a target protein) from the extracellular space (the extracellular milieu) of a cell by sequestering the target protein in the cell's lysosome and degrading the target protein.

Removal of a target protein may refer to reduction, or depletion, of the target protein from the cell surface or from the extracellular space, or the extracellular milieu, that is, a reduction, or depletion, of the amount of the target protein on the cell surface or in the extracellular milieu. In some embodiments, the method is a method of reducing the amount or level of a target protein in a biological system or cellular sample.

In one aspect, provided herein are methods of using the conjugates described herein to sequester a polypeptide of interest (a target protein) in a cell's lysosome. In one aspect, provided herein are methods of using the conjugates described herein to sequester a polypeptide of interest (a target protein) in a cell's lysosome and to degrade the the polypeptide of interest.

In one aspect, provided herein are methods of using the conjugates described herein to degrade a polypeptide of interest (a target protein).

In one aspect, provided herein are methods of depleting a polypeptide of interest (a target protein) described herein by degradation through a cell's lysosomal pathway.

In another aspect, provided herein are methods of depleting a polypeptide of interest (a target protein) described herein by administering to a subject in need thereof an effective amount of a conjugate or pharmaceutically acceptable salt described herein, or a pharmaceutical composition described herein. In certain embodiments, the subject is a mammal (e.g., human).

In certain embodiments, the target protein is a membrane bound protein. In certain embodiments, the target protein is an extracellular protein.

In certain embodiments, the target protein is a VEGF protein, an EGFR protein, a VEGFR protein, a PD-L1 protein, an FGFR2 protein or an FGFR3 protein.

In another aspect, provided herein are methods of treating a disease or disorder by administering to a subject, e.g., a human, in need thereof an effective amount of a conjugate or pharmaceutically acceptable salt described herein, or a pharmaceutical composition described herein.

The terms “administer”, “administration”, or “administering” refer to the act of injecting or otherwise physically delivering a substance (e.g., a conjugate or pharmaceutical composition provided herein) to a subject or a patient (e.g., human), such as by mucosal, topical, intradermal, parenteral, intravenous, intramuscular delivery and/or any other method of physical delivery described herein or known in the art. In a particular embodiment, administration is by intravenous infusion.

The terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result.

In certain embodiments, when the disorder or disease is cancer, “effective amount” or “therapeutically effective amount” mean that amount of a conjugate or pharmaceutical composition provided herein which, when administered to a human suffering from a cancer, is sufficient to effect treatment for the cancer. “Treating” or “treatment” of the cancer includes one or more of:

(1) limiting/inhibiting growth of the cancer, e.g. limiting its development;
(2) reducing/preventing spread of the cancer, e.g. reducing/preventing metastases;
(3) relieving the cancer, e.g. causing regression of the cancer,
(4) reducing/preventing recurrence of the cancer; and
(5) palliating symptoms of the cancer.

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

In certain embodiments, the disease or disorder is treated by depletion of the target protein by degradation through the lysosomal pathway.

In certain embodiments, the disease or disorder is treated by depletion of certain proteins, for example, soluble proteins, e.g., secreted proteins, cell surface proteins (for example, cell surface receptor proteins, e.g., tyrosine kinase receptors, soluble cytokine receptors, and immune checkpoint receptors, e.g., EGFR, VEGFR, FGFR, and PD-L1), lectins, complements, lipoproteins, transport proteins, MHC class I and class II molecules, cytokines, chemokines, and/or receptors, or fragments or subunits of any of the foregoing.

In certain embodiments, the disease or disorder is a cancer.

In certain embodiments, the cancer is selected from the group consisting of bladder cancer, breast cancer, cervical cancer, cholangiocarcinoma, endometrial cancer, hepatocellular carcinoma, kidney cancer, melanoma, myeloid neoplasms, non-small cell lung cancer (NSCLC), Ewing's sarcoma, and Hodgkin's Lymphoma.

In certain embodiments, the cancer is a solid tumor.

In certain embodiments, the disease or disorder is an inflammatory or autoimmune disease.

In certain embodiments, the disease or disorder is an inflammatory disease.

In certain embodiments, the disease or disorder is an autoimmune disease.

### Definitions

The terms “protein” and “polypeptide” are used interchangeably. Proteins may include moieties other than amino acids (e.g., may be glycoproteins, etc.) and/or may be otherwise processed or modified. Those of ordinary skill in the art will appreciate that a “protein” can be a complete protein chain as produced by a cell (with or without a signal sequence), or can be a protein portion thereof. Those of ordinary skill will appreciate that a protein can sometimes include more than one protein chain, for example non-covalently or covalently attached, e.g., linked by one or more disulfide bonds or associated by other means. Polypeptides may contain I-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

The terms “antibody” and “immunoglobulin” are terms of art and can be used interchangeably herein, and refer to a molecule with an antigen binding site that specifically binds an antigen.

In a certain embodiments, an isolated antibody (e.g., monoclonal antibody) described herein, or an antigen-binding fragment thereof, which specifically binds to a protein of interest, for example, EGFR, is conjugated to one or more lysosomal targeting moieties, for example, via a linker.

An “antigen” is a moiety or molecule that contains an epitope to which an antibody can specifically bind. As such, an antigen is also is specifically bound by an antibody. In a specific embodiment, the antigen, to which an antibody described herein binds, is a protein of interest, for example, EGFR (e.g., human EGFR), or a fragment thereof, or for example, an extracellular domain of EGFR (e.g., human EGFR).

An “epitope” is a term known in the art and refers to a localized region of an antigen to which an antibody can specifically bind. An epitope can be a linear epitope of contiguous amino acids or can comprise amino acids from two or more non-contiguous regions of the antigen.

The terms “binds,” “binds to,” “specifically binds” or “specifically binds to” in the context of antibody binding refer to antibody binding to an antigen (e.g., epitope) as such binding is understood by one skilled in the art. For example, a molecule that specifically binds to an antigen may bind to other polypeptides, generally with lower affinity as determined by, e.g., immunoassays, Biacore™, KinExA 3000 instrument (Sapidyne Instruments, Boise, Id.), or other assays known in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins. In another specific embodiment, where EGFR is the protein of interest, molecules that specifically bind to an antigen do not cross react with other non-EGFR proteins.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

In a particular embodiment, an antibody is a 4-chain antibody unit comprising two heavy (H) chain/light (L) chain pairs, wherein the amino acid sequences of the H chains are identical and the amino acid sequences of the L chains are identical. In a specific embodiment, the H and L chains comprise constant regions, for example, human constant regions. In a yet more specific embodiment, the L chain constant region of such antibodies is a kappa or lambda light chain constant region, for example, a human kappa or lambda light chain constant region. In another specific embodiment, the H chain constant region of such antibodies comprise a gamma heavy chain constant region, for example, a human gamma heavy chain constant region. In a particular embodiment, such antibodies comprise IgG constant regions, for example, human IgG constant regions.

The term “constant region” or “constant domain” is a well-known antibody term of art (sometimes referred to as “Fc”), and refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

The term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

The term “monoclonal antibody” is a well-known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody specifically binds to an epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody).

The terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain. Variable regions comprise complementarity determining regions (CDRs) flanked by framework regions (FRs). Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196: 901-917; Al-Lazikani et al., 1997, J. Mol. Biol., 273: 927-948; Chothia et al., 1992, J. Mol. Biol., 227: 799-817; Tramontano et al., 1990, J. Mol. Biol. 215(1):175-82; U.S. Pat. No. 7,709,226; and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)); or (iii) the ImMunoGeneTics (IMGT) numbering system, for example, as described in Lefranc, 1999, The Immunologist, 7: 132-136 and Lefranc et al., 1999, Nucleic Acids Res., 27: 209-212 (“IMGT CDRs”); or (iv) the AbM numbering system, which will be referred to herein as the “AbM CDRs”, for example as described in MacCallum et al., 1996, J. Mol. Biol., 262: 732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001); or (v) the Contact numbering system, which will be referred to herein as the “Contact CDRs” (the Contact definition is based on analysis of the available complex crystal structures (bioinf.org.uk/abs) (see, e.g., MacCallum et al., 1996, J. Mol. Biol., 262:732-745)).

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen. In another aspect, an antibody fragment, such as an antibody fragment that comprises the Fc region, retains at least one of the biological functions normally associated with the Fc region when present in an intact antibody. Such functions may include FcRn binding, antibody half life modulation, conjugate function and complement binding. In another aspect, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

“Alkyl” means a straight or branched saturated hydrocarbon group containing from 1-10 carbon atoms, and in certain embodiments includes 1-6 carbon atoms. In certain embodiments, alkyl includes 1-4 carbon atoms (“C₁₋₄ alkyl”). In certain embodiments alkyl includes 1-3 carbon atoms (“C₁₋₃ alkyl”). In certain embodiments, alkyl includes methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, or n-decyl.

“Alkylene” means a straight or branched saturated divalent hydrocarbon group containing from 1-10 carbon atoms. In certain embodiments, alkylene includes 1-6 carbon atoms (“C₁₋₆ alkylene”).

“Halo” means a fluoro, chloro, bromo, or iodo group.

“CN” means a cyano group.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷C, ¹⁸C, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. In certain embodiments, where an integer is required, the term “about” means within plus or minus 10% of a given value or range, rounded either up or down to the nearest integer.

In the description herein, if there is any discrepancy between a chemical name and chemical structure, the chemical structure shall prevail.

### ADDITIONAL EMBODIMENTS

Aspects of the present disclosure are described in the follow clauses.

Clause 1. A cell surface mannose-6-phosphate receptor (M6PR) binding compound of formula (XI):

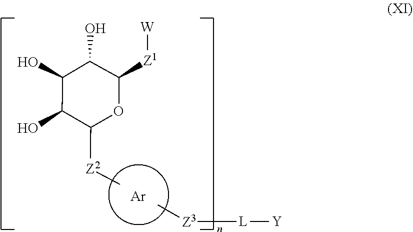

or a salt thereof,
wherein:

each W is independently a hydrophilic head group;

each Z¹ is independently selected from optionally substituted (C₁-C₃)alkylene and optionally substituted ethenylene;

each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;

each Ar is independently an optionally substituted aryl or heteroaryl linking moiety (e.g., monocyclic or bicyclic aryl or heteroaryl, optionally substituted);

each Z³ is independently a linking moiety;

n is 1 to 500;

L is a linker; and

Y is a moiety of interest;

wherein when m is 1 and Ar is phenyl, then: i) L comprises a backbone of at least 16 consecutive atoms; ii) Y is a biomolecule; and/or ii) Z³ is amide, sulfonamide, urea or thiourea.

Clause 2. The compound of clause 1, wherein each Ar is independently selected from optionally substituted phenyl, optionally substituted pyridyl, optionally substituted biphenyl, optionally substituted naphthalene, optionally substituted triazole and optionally substituted phenylene-triazole.

Clause 3. The compound of clause 2, wherein Ar is selected from optionally substituted 1,4-phenylene, optionally substituted 1,3-phenylene, or optionally substituted 2,5-pyridylene.

Clause 4. The compound of clause 3, wherein the compound is of formula (XIIa) or (XIIb):

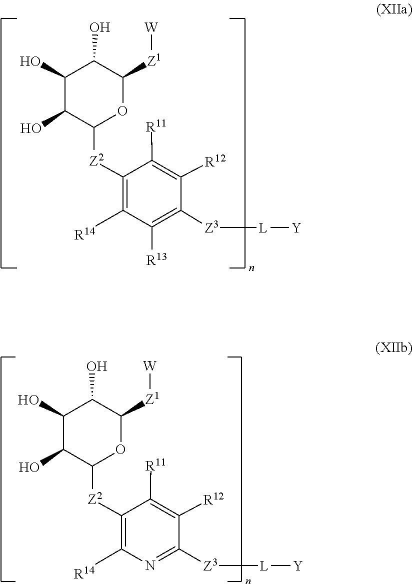

or a salt thereof,
wherein:

each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each R²⁵ is independently selected from H, and optionally substituted (C₁-C₆)alkyl.

Clause 5. The compound of clause 1, wherein Ar is an optionally substituted fused bicyclic aryl or fused bicyclic heteroaryl.

Clause 6. The compound of clause 5, wherein Ar is optionally substituted naphthalene or an optionally substituted quinoline.

Clause 7. The compound of clause 6, wherein the compound is of formula (XIIIa) or (XIIIb):

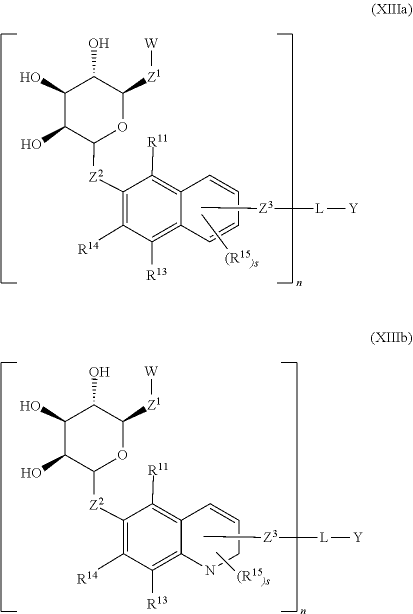

or a salt thereof,
wherein:

each R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;

s is 0 to 3; and

each R²⁵ is independently selected from H, and optionally substituted (C₁-C₆)alkyl.

Clause 8. The compound of clause 7, wherein the compound is of one of formula (XIIIc) to (XIIIh):

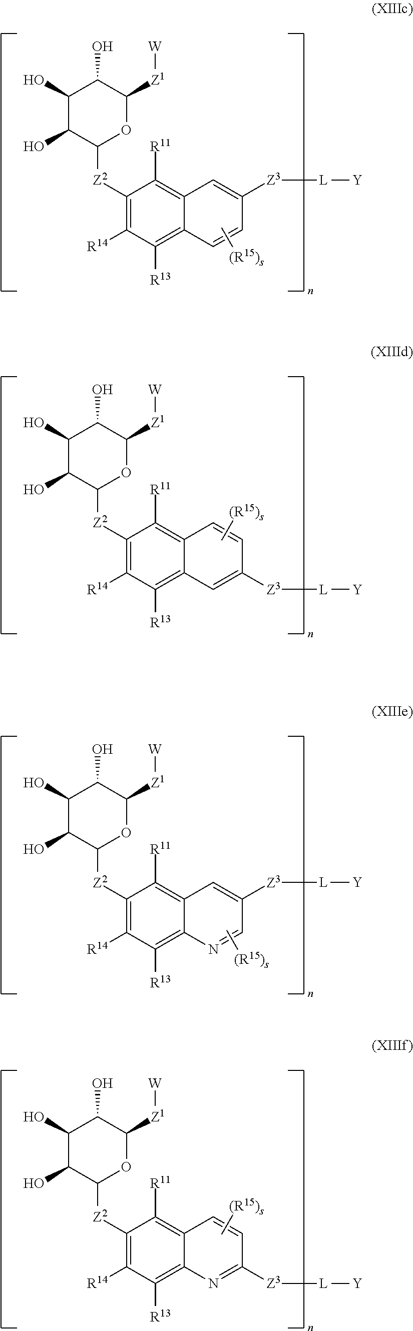

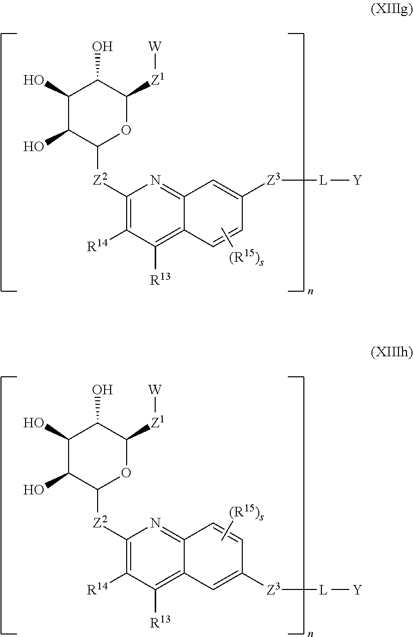

or a salt thereof.

Clause 9. The compound of clause 1, wherein Ar is optionally substituted bicyclic aryl or optionally substituted bicyclic heteroaryl and wherein the compound is of formula (XIVa)

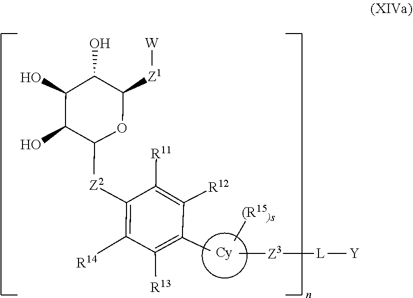

or a salt thereof,
wherein:

each Cy is independently monocyclic aryl or monocyclic heteroaryl;

each R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;

s is 0 to 4; and

each R²⁵ is independently selected from H, and optionally substituted (C₁-C₆)alkyl.

Clause 10. The compound of clause 9, wherein Ar is optionally substituted biphenyl, Cy is optionally substituted phenyl, and the compound is of formula (XIVb):

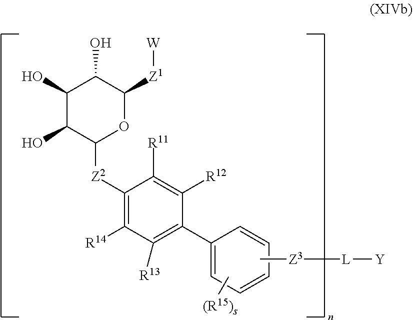

or a salt thereof.

Clause 11. The compound of clause 10, wherein the compound is of formula (XIVc) or (XIVd):

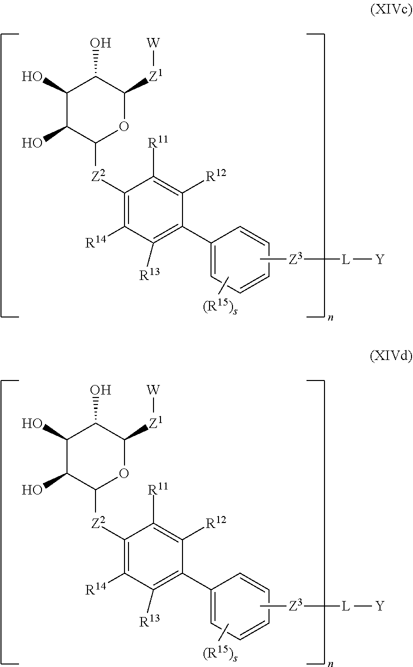

or a salt thereof.

Clause 12. The compound of any one of clauses 1 to 10, wherein Ar is substituted with at least one OH substituent.

Clause 13. The compound of any one of clauses 4, 6, 7, 9 and 10, wherein R¹¹ to R¹⁵ are each H.

Clause 14. The compound of any one of clauses 4, 6, 7, 9 and 10, wherein at least one of R¹¹ to R¹⁵ is OH (e.g., at least two are OH).

Clause 15. The compound of any one of clauses 1 to 14, wherein:

Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—.

X¹ and X² are selected from O, S and NR²³; and

R²³ and R²⁴ are independently selected from H, C₍₁₋₃₎-alkyl (e.g., methyl) and substituted C₍₁₋₃₎-alkyl.

Clause 16. The compound of any one of clauses 1 to 15, wherein Z³ is

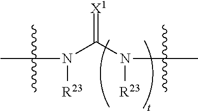

wherein:

X¹ is O or S;

t is 0 or 1; and

each R²³ is independently selected from H, C₍₁₋₃₎-alkyl (e.g., methyl) and substituted C₍₁₋₃₎-alkyl.

Clause 17. The compound of clause 16, wherein Z³ is —NHC(═X¹)NH—, wherein X¹ is O or S.

Clause 18. The compound of any one of clauses 1 to 14, wherein Ar is triazole and the compound is of formula (XIIc) or (XIId):

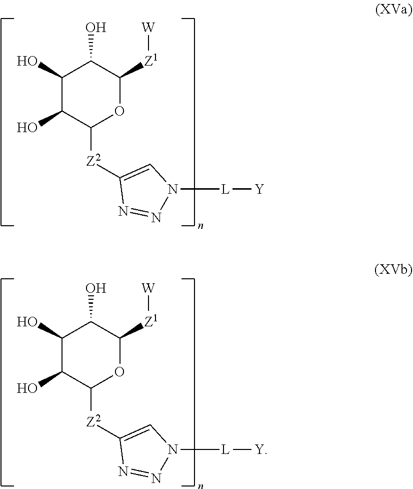

Clause 19. The compound of clause 18, wherein Z³ is optionally substituted triazole and the compound is of formula (XIIc) or (XIId):

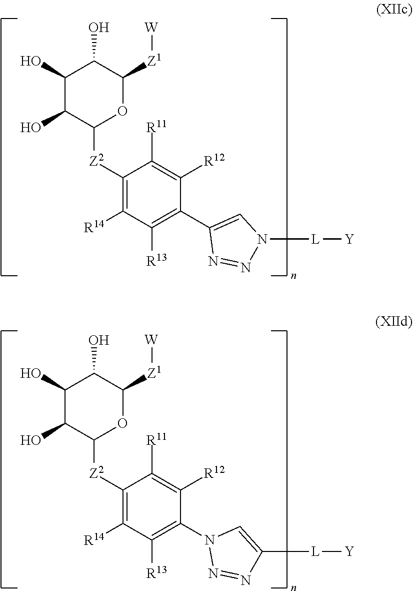

or a salt thereof,
wherein:

each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and

each R²⁵ is independently selected from H, and optionally substituted (C₁-C₆)alkyl.

Clause 20. The compound of any one of clauses 1 to 19, wherein —Ar—Z³— is selected from:

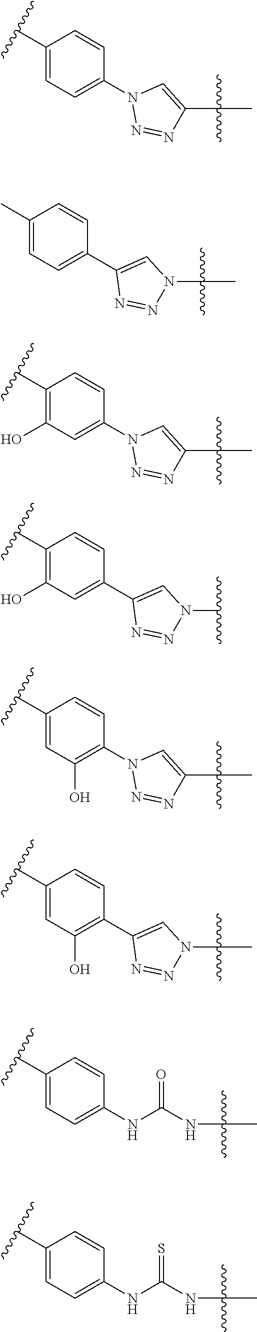

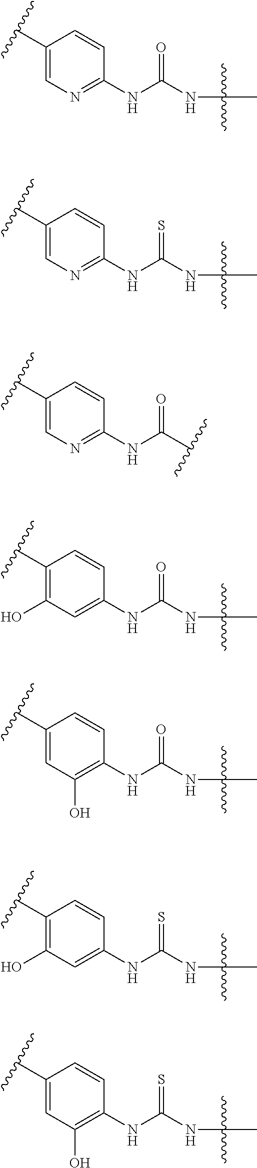

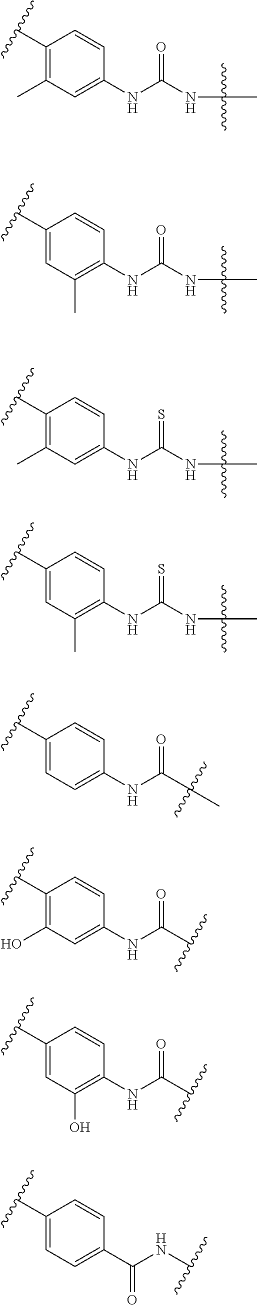

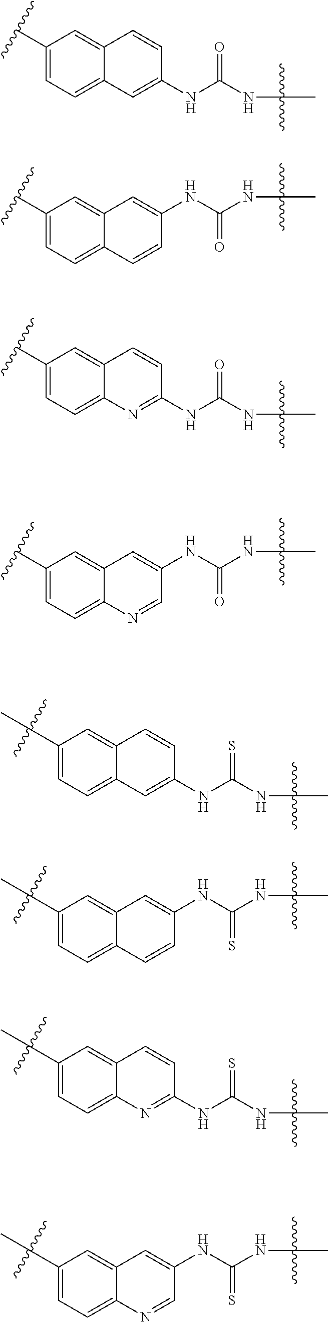

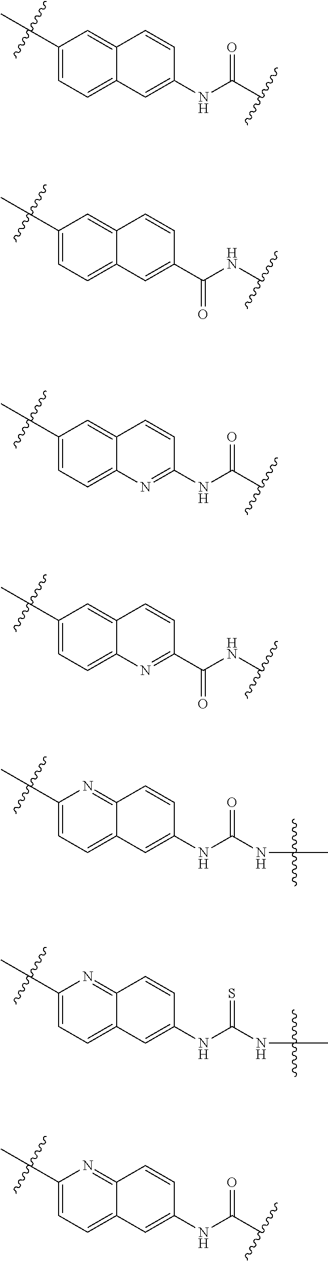

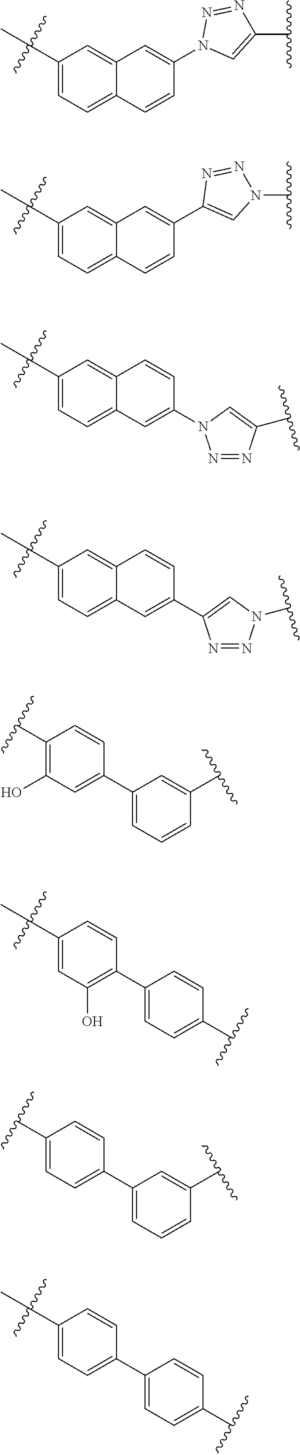

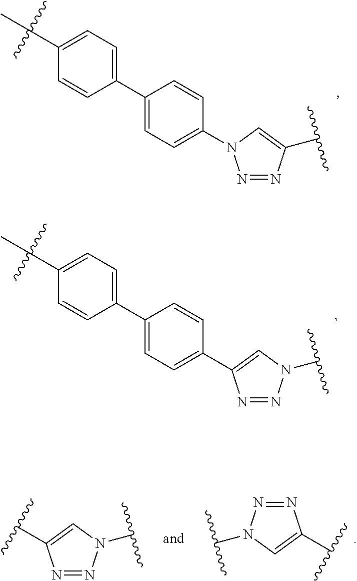 [SMILES: *c1ccc(-c2ccc(-n3cc(*)nn3)cc2)cc1 ; *c1ccc(-c2ccc(-c3cn(*)nn3)cc2)cc1 ; *c1cn(*)nn1 ; *c1cn(*)nn1]

Clause 21. The compound of any one of clauses 1 to 20, wherein m is at least 2, and L is a branched linker that covalently links each Ar group to Y.

Clause 22. The compound of clause 21, wherein m is 2 to 20 (e.g., m is 2 to 6, such as 2 or 3).

Clause 23. The compound of clause 21, wherein:

m is 20 to 500 (e.g., 20 to 400, 20 to 300, or 20 to 200, or 50 to 500, or 100 to 500); and

L is an α-amino acid polymer (e.g., poly-L-lysine) wherein a multitude of —Ar—Z³— groups are covalently linked to the polymer backbone via sidechain groups (e.g., via conjugation to the sidechain amino groups of lysine residues).

Clause 24. The compound of any one of clauses 21 to 23, wherein m is at least 2 and each Z³ linking moiety is separated from every other Z³ linking moiety by a chain of at least 16 consecutive atoms via linker L (e.g., by a chain of at least 20, at least 25, or at least 30 consecutive atoms, and in some cases by a chain of up to 100 consecutive atoms).

Clause 25. The compound of any one of clauses 1 to 24, wherein the compound is of formula (XV):

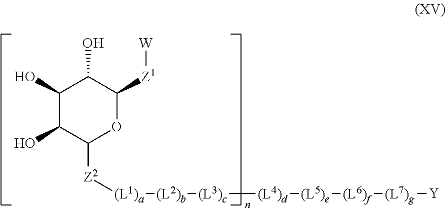

or a salt thereof,
wherein:

n is 1 to 500 (e.g., n is 1 to 20, 1 to 10, 1 to 6 or 1 to 5);

each L¹ to L⁷ is independently a linking moiety that together provide a linear or branched linker between the n Z² groups and Y, and wherein -(L¹)ₐ- comprises the linking moiety Ar that is optionally substituted aryl or heteroaryl group;

a is 1 or 2; and

b, c, d, e, f, and g are each independently 0, 1, or 2.

Clause 26. The compound of clause 25, wherein the linear or branched linker separates each Z² and Y by a chain of at least 16 consecutive atoms (e.g., at least 20 consecutive atoms, at least 30 consecutive atoms, or 16 to 100 consecutive atoms).

Clause 27. The compound of any one of clauses 25 to 26, wherein n is 1 to 20.

Clause 28. The compound of any one of clauses 25 to 27, wherein n is at least 2 (e.g., n is 2 or 3).

Clause 29. The compound of clause 28, wherein d is >0 and L⁴ is a branched linking moiety that is covalently linked to each L¹ linking moiety.

Clause 30. The compound of any one of clauses 25 to 29, wherein the compound is of formula (XVIa)

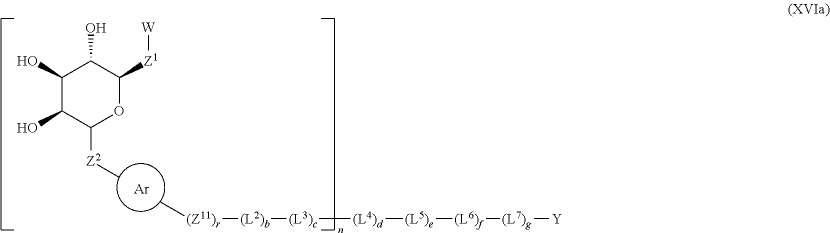

wherein:

Ar is an optionally substituted aryl or heteroaryl group (e.g., monocyclic or bicyclic or tricyclic aryl or heteroaryl group);

Z¹¹ is a linking moiety (e.g., covalent bond, heteroatom, group having a backbone of 1-3 atoms in length or triazole);

r is 0 or 1; and

n is 1 to 6.

Clause 31. The compound of clause 30, wherein Ar is selected from optionally substituted phenyl, optionally substituted pyridyl, optionally substituted biphenyl, optionally substituted naphthalene, optionally substituted quinoline, optionally substituted triazole, optionally substituted phenyl-triazole, optionally substituted biphenyl-triazole, and optionally substituted naphthalene-triazole.

Clause 32. The compound of clause 31, wherein Ar is optionally substituted 1,4-phenylene.

Clause 33. The compound of any one of clauses 30 to 32, wherein Ar substituted with at least one hydroxy.

Clause 34. The compound of any one of clauses 25 to 33, wherein L¹ or —Ar—(Z¹¹)ᵣ— is selected from:

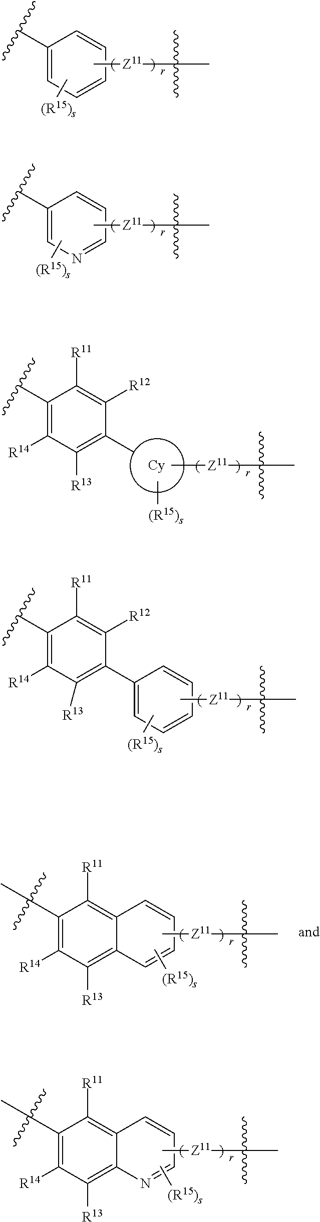

wherein:

Cy is monocyclic aryl or heteroaryl;

r is 0 or 1;

s is 0 to 4;

R¹¹ to R¹⁴ and each R¹⁵ are independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —OCOR²⁵, —CONHR²⁵, and —NHCOR²⁵, wherein each R²⁵ is independently selected from H, C₍₁₋₆₎-alkyl and substituted C₍₁₋₆₎-alkyl; and

Z¹¹ is selected from covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X²— and optionally substituted triazole, where X¹ and X² are selected from O, S and NR²³, wherein R²³ and R²⁴ are independently selected from H, C₍₁₋₃₎-alkyl (e.g., methyl) and substituted C₍₁₋₃₎-alkyl.

Clause 35. The compound of clause 34, wherein L¹ is

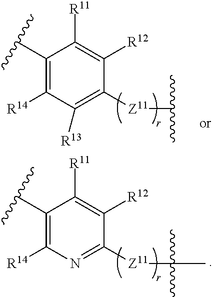

Clause 36. The compound of clause 34, wherein L¹ is

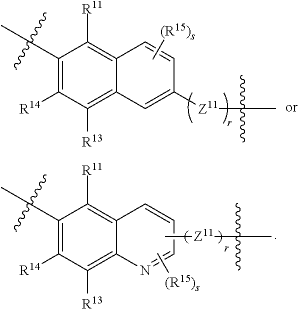

Clause 37. The compound of clause 34, wherein L¹ is selected from:

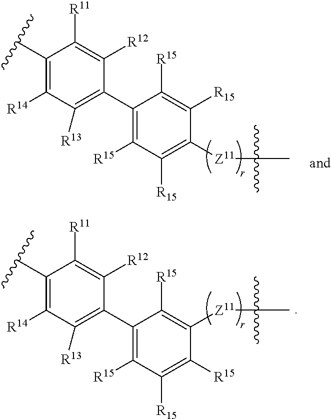

Clause 38. The compound of any one of clauses 34 to 37, wherein r is 0.

Clause 39. The compound of any one of clauses 34 to 37, wherein r is 1 and Z¹¹ is selected from —O—, —NR²³—, —NR²³CO—, CONR²³—, —NR²³CO₂—, —OCONR²³—, —NR²³C(═X¹)NR²³—, —CR²⁴═N—, and —CR²⁴═N—X²—, wherein X¹ and X² are selected from O, S and NR²³, and each R²³ and R²⁴ is independently selected from H, C₍₁₋₃₎-alkyl (e.g., methyl) and substituted C₍₁₋₃₎-alkyl.

Clause 40. The compound of any one of clauses 34 to 37, wherein r is 1 and Z¹¹ is

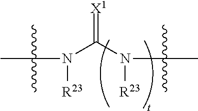

wherein:

X¹ is O or S;

t is 0 or 1; and

each R²³ is independently selected from H, C₍₁₋₃₎-alkyl (e.g., methyl) and substituted C₍₁₋₃₎-alkyl.

Clause 41. The compound of clause 40, wherein Z¹¹ is —NHC(═X¹)NH—, wherein X¹ is O or S.

Clause 42. The compound of any one of clauses 34 to 37, wherein r is 1 and Z¹¹ is triazole.

Clause 43. The compound of any one of clauses 1 to 42, wherein Y is selected from small molecule, dye, fluorophore, monosaccharide, disaccharide, trisaccharide, and chemoselective ligation group or precursor thereof.

Clause 44. The compound of any one of clauses 1 to 42, wherein Y is a biomolecule.

Clause 45. The compound of clause 44, wherein the biomolecule is selected from peptide, protein, polynucleotide, polysaccharide, glycoprotein, lipid, enzyme, antibody, and antibody fragment.

Clause 46. The compound of any one of clauses 1 to 45, wherein Y is a moiety that specifically binds a target protein.

Clause 47. The compound of clause 46, wherein the target protein is a membrane bound protein.

Clause 48. The compound of clause 46, wherein the target protein is an extracellular protein.

Clause 49. The compound of any one of clauses 46 to 49, wherein Y is selected from antibody, antibody fragment (e.g., antigen-binding fragment of an antibody), chimeric fusion protein, an engineered protein domain, D-protein binder of target protein, aptamer, peptide, enzyme substrate and small molecule inhibitor or ligand.

Clause 50. The compound of clause 49, wherein Y is antibody or antibody fragment that specifically binds the target protein and the compound is of formula (Va):

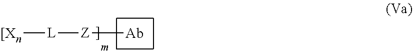

or a pharmaceutically acceptable salt thereof,
wherein:

n is 1 to 20;

m is an average loading of 1 to 80;

Ab is the antibody or antibody fragment that specifically binds the target protein; and

Z is a residual moiety resulting from the covalent linkage of a chemoselective ligation group to a compatible group of Ab.

Clause 51. The compound of clause 49, wherein Y is a small molecule inhibitor or ligand of the target protein.

Clause 52. The compound of any one of clauses 1 to 51, wherein the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

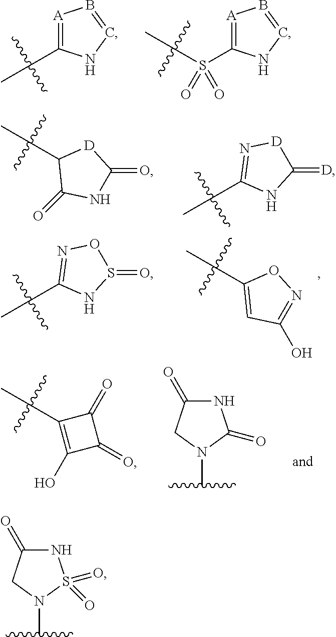

or a salt thereof,
wherein:

R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);

A, B, and C are each independently CH or N; and

D is each independently O or S.

Clause 53. The compound of clause 52, wherein W is selected from —P═O(OH)₂, —SO₃H, —COOH and —CH(COOH)₂, or a salt thereof.

Clause 54. The compound of any one of clauses 1 to 53, wherein:

Z¹ is —(CH₂)ⱼ— or —(C(R²²)₂)ⱼ—, wherein each R²² is independently selected from H, halogen (e.g., F) and optionally substituted (C₁-C₆)alkyl; and j is 1 to 3.

Clause 55. The compound of any one of clauses 1 to 53, wherein Z¹ is —CH═CH—.

Clause 56. The compound of any one of clauses 1 to 55, wherein Z² is O or S.

Clause 57. The compound of any one of clauses 1 to 55, wherein Z² is —NR²¹—.

Clause 58. The compound of any one of clauses 1 to 55, wherein Z² is —C(R²²)₂—, wherein each R²² is independently selected from H, halogen (e.g., F) and optionally substituted (C₁-C₆)alkyl.

Clause 59. The compound of any one of clauses 1 to 53, wherein:

Z¹ is selected from —(CH₂)ⱼ—, substituted (C₁-C₃)alkylene and —CH═CH—;

j is 1 to 3; and

Z² is selected from O and CH₂.

Clause 60. The compound of clause 60, wherein:

Z¹ is —(CH₂)₂—, —CH₂—CF₂— or —CH₂—CHF—; and

Z² is O.

Clause 61. The compound of clause 60, wherein:

Z¹ is —(CH₂)₂—, —CH₂—CF₂— or —CH₂—CHF—; and

Z² is CH₂.

Clause 62. The compound of clause 60, wherein:

Z¹ is —CH═CH—; and

Z² is O.

Clause 63. The compound of clause 60, wherein:

Z¹ is —CH═CH—; and

Z² is CH₂.

Clause 64. The compound of any one of clauses 1 to 63, wherein X is selected from:

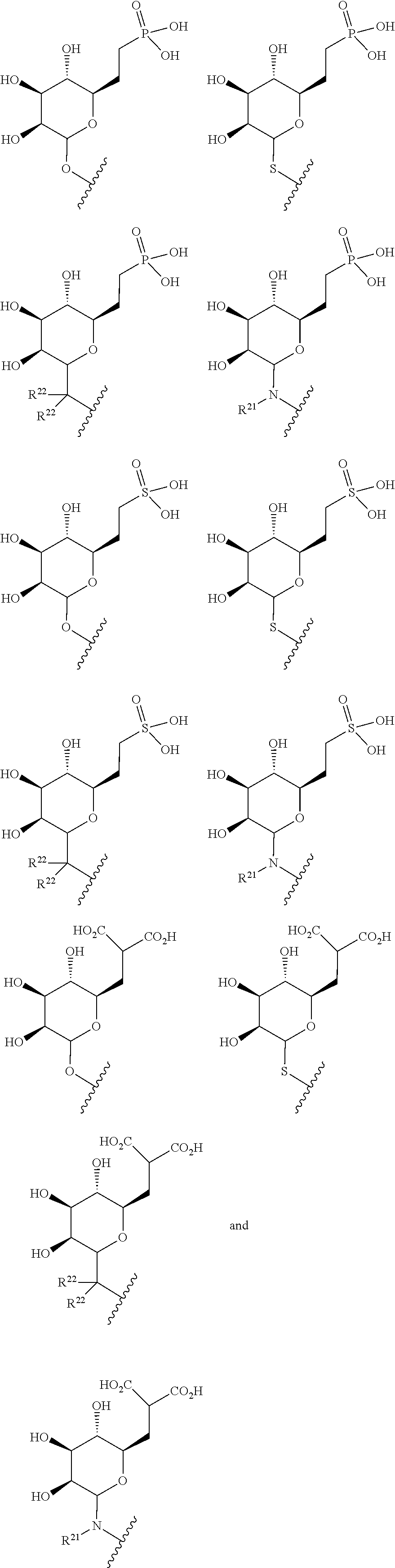

Clause 65. The compound of any one of clauses 25 to 64, wherein n is 1 to 6 (e.g., n is 1 to 5, or 2 to 6, or 1, 2 or 3), and wherein:

when d is 0, n is 1;

when d is 1, n is 1 to 3; and

when d is 2, n is 1 to 6.

Clause 66. The compound of any one of clauses 25 to 65, wherein:

each L² is independently selected from —C₁₋₆-alkylene-, —NHCO—C₁₋₆-alkylene-, —CONH—C₁₋₆-alkylene-, —O(CH₂)ₚ—, and —(OCH₂CH₂)ₚ—, wherein p is 1 to 10; and

each L³ is independently selected from:

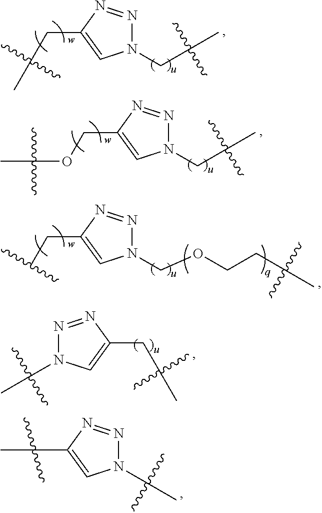

and —(OCH₂CH₂)_{q}—, wherein q is 1 to 10, u is 0 to 10, and w is 1 to 10.

Clause 67. The compound of any one of clauses 25 to 66, wherein when n is 2 or more, at least one L⁴ is present and is a branched linking moiety.

Clause 68. The compound of any one of clauses 25 to 67, wherein each L⁴ is independently selected from: —OCH₂CH₂—,

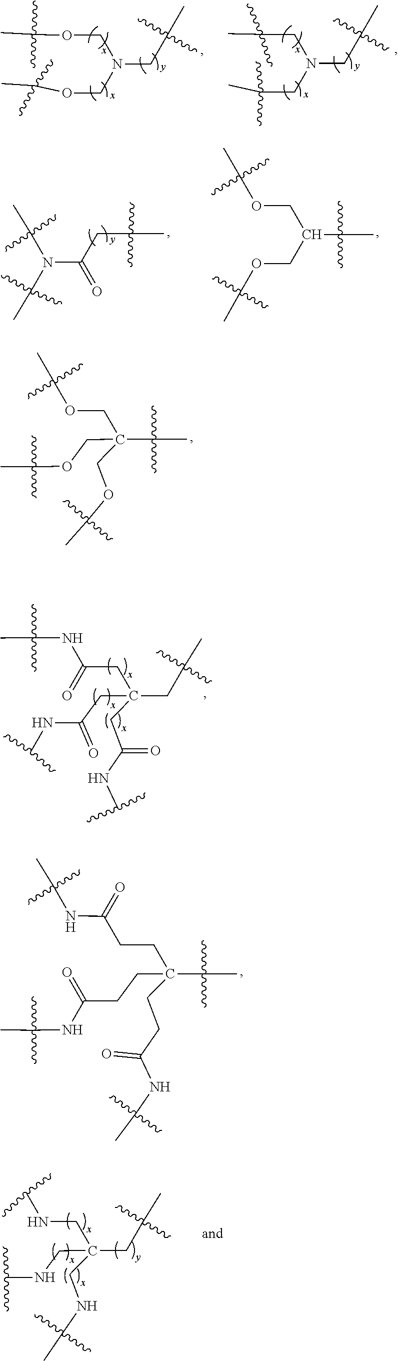

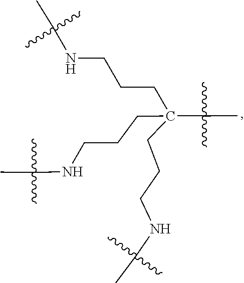

wherein each x and y are each independently 1 to 10.

Clause 69. The compound of any one of clauses 25 to 68, wherein:

each L⁵ is independently —NHCO—C₁₋₆-alkylene-, —CONH—C₁₋₆-alkylene-, —C₁₋₆-alkylene-,

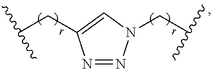

or —(OCH₂CH₂)ᵣ—;

each L⁶ is independently —NHCO—C₁₋₆-alkylene-, —CONH—C₁₋₆-alkylene-, —C₁₋₆-alkylene-, or —(OCH₂CH₂)ₛ—;

each L⁷ is independently —NHCO—C₁₋₆-alkylene-, —CONH—C₁₋₆-alkylene-, —C₁₋₆-alkylene-, —(OCH₂CH₂)ₜ—, or —OCH₂—; and

r, s, and t are each independently 1 to 20.

Clause 70. The compound of any one of clauses 25 to 69, wherein a is 1.

Clause 71. The compound of any one of clauses 25 to 70, wherein at least one of b, c, e, f, and g is not 0.

Clause 72. The compound of any one of clauses 25 to 71, wherein at least one of b or c is not 0 and at least one of e, f, and g is not 0.

Clause 73. The compound of any one of clauses 25 to 72, wherein a, b, and c are each independently 1 or 2.

Clause 74. The compound of any one of clauses 1 to 73, wherein the linker L is selected from any one of the structures of Tables 2-3.

Clause 75. The compound of any one of clauses 1 to 74, wherein the compound is selected from the compounds of Tables 5-9.

Clause 76. A cell surface receptor binding conjugate of formula (I):

Xₙ-L-Y (I)

or a salt thereof,
wherein:

X is a moiety that binds to a cell surface asialoglycoprotein receptor (ASGPR) or a moiety that binds to a cell surface mannose-6-phosphate receptor (M6PR);

n is 1 to 500 (e.g., n is 1 to 20, 1 to 10, 1 to 6 or 1 to 5); and

L is a linker;

Y is a biomolecule that specifically binds a target protein.

Clause 77. The conjugate of clause 76, wherein the conjugate is formula (V):

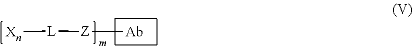

or a pharmaceutically acceptable salt thereof,
wherein:

n is 1 to 20;

m is an average loading of 1 to 80;

Ab is an antibody or antibody fragment that specifically binds the target protein; and

Z is a residual moiety resulting from the covalent linkage of a chemoselective ligation group to a compatible group of Ab.

Clause 78. The conjugate of clause 76 or 77, wherein n is 1 to 6.

Clause 79. The conjugate of clause 76 or 77, wherein n is 2 or less.

Clause 80. The conjugate of clause 79, wherein n is 1.

Clause 81. The conjugate of clause 76 or 77, wherein n is at least 2.

Clause 82. The conjugate of clause 81, wherein n is 2.

Clause 83. The conjugate of clause 81, wherein n is 3.

Clause 84. The conjugate of clause 81, wherein n is 4.

Clause 85. The conjugate of any one of clauses 76 to 84, wherein m is 1 to 20.

Clause 86. The conjugate of any one of clauses 76 to 84, wherein m is 1 to 12.

Clause 87. The conjugate of any one of clauses 76 to 86, wherein m is at least about 2.

Clause 88. The conjugate of any one of clauses 76 to 86, wherein m is at least about 3.

Clause 89. The conjugate of any one of clauses 76 to 86, wherein m is at least about 4.

Clause 90. The conjugate of any one of clauses 77 to 89, wherein Z is a residual moiety resulting from the covalent linkage of a thiol-reactive chemoselective ligation group to one or more cysteine residue(s) of Ab.

Clause 91. The conjugate of any one of clauses 76 to 89, wherein Z is a residual moiety resulting from the covalent linkage of an amine-reactive chemoselective ligation group to one or more lysine residue(s) of Ab.

Clause 92. The conjugate of any one of clauses 76 to 91, wherein X is a moiety that binds M6PR and is of the formula:

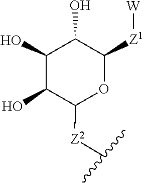

or a salt thereof,
wherein:

each W is independently a hydrophilic head group;

each Z¹ is independently selected from optionally substituted (C₁-C₃)alkylene and optionally substituted ethenylene; and

each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl. Clause 93. The conjugate of clause 92, wherein the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H,

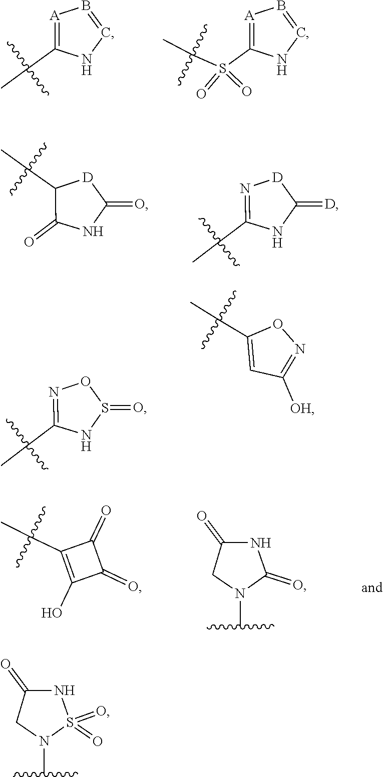

or a salt thereof,
wherein:

R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);

A, B, and C are each independently CH or N; and

D is each independently O or S.

Clause 94. The conjugate of clause 93, wherein W is selected from —P═O(OH)₂, —SO₃H, —CO₂H and —CH(CO₂H)₂, or a salt thereof.

Clause 95. The conjugate of any one of clauses 92 to 94, wherein Z¹ is —(CH₂)ᵣ— and j is 1 to 3.

Clause 96. The conjugate of any one of clauses 92 to 95, wherein Z¹ is —CH═CH—.

Clause 97. The conjugate of any one of clauses 92 to 96, wherein Z² is 0 or S.

Clause 98. The conjugate of any one of clauses 92 to 96, wherein Z² is —NR²¹—.

Clause 99. The conjugate of any one of clauses 92 to 96, wherein Z² is —C(R²²)₂—.

Clause 100. The conjugate of any one of clauses 92 to 94, wherein:

Z¹ is selected from —(CH₂)—, substituted (C₁-C₃)alkylene and —CH═CH—;

j is 1 to 3; and

Z² is selected from O and CH₂.

Clause 101. The conjugate of clause 100, wherein:

Z¹ is —(CH₂)₂—, —CH₂—CF₂— or —CH₂—CHF—; and

Z² is O.

Clause 102. The conjugate of clause 100, wherein:

Z¹ is —(CH₂)₂—, —CH₂—CF₂— or —CH₂—CHF—; and

Z² is CH₂.

Clause 103. The conjugate of clause 100, wherein: Z¹ is —CH═CH—; and Z² is O.

Clause 104. The conjugate of clause 100, wherein: Z¹ is —CH═CH—; and Z² is CH₂.

Clause 105. The conjugate of any one of clauses 92 to 104, wherein X is selected from:

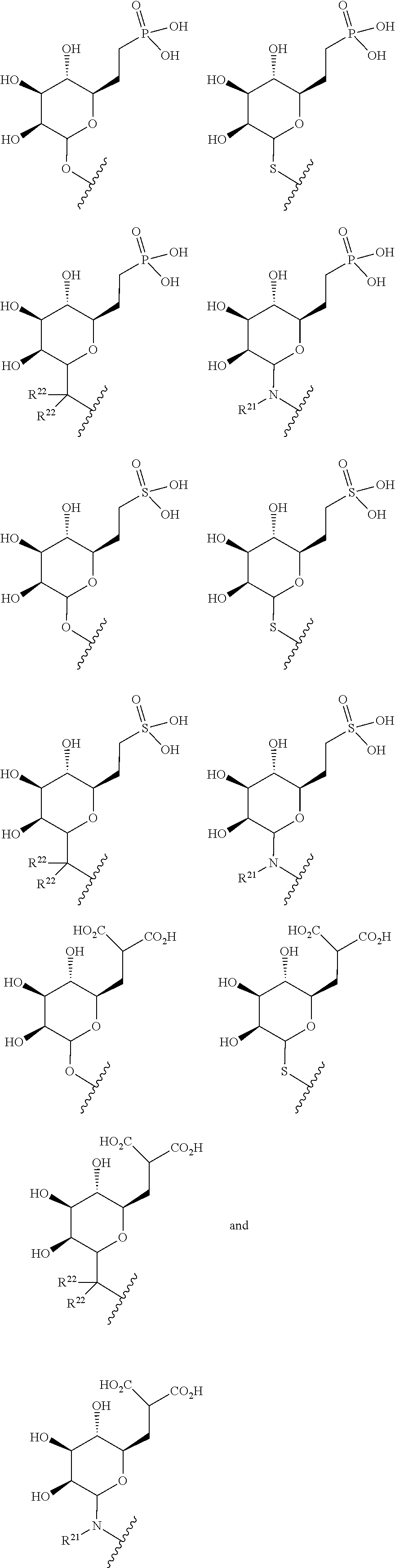

Clause 106. The conjugate of any one of clauses 76 to 91, wherein X is a moiety that binds to ASGPR and is selected from formula (III-a) to (III-j):

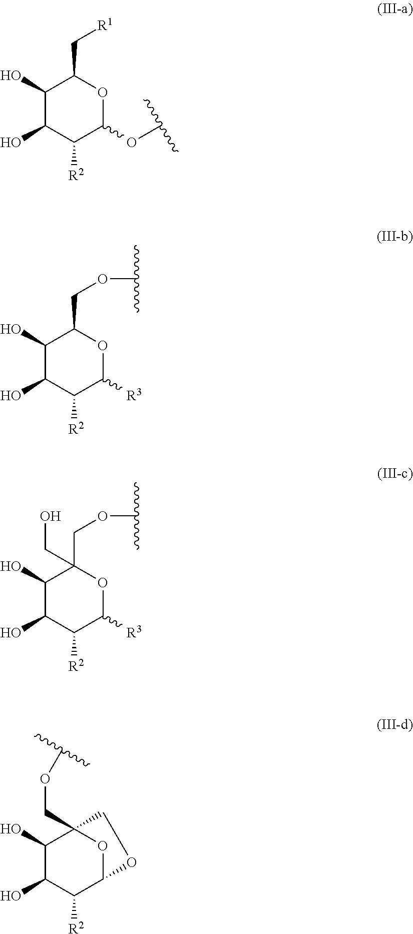

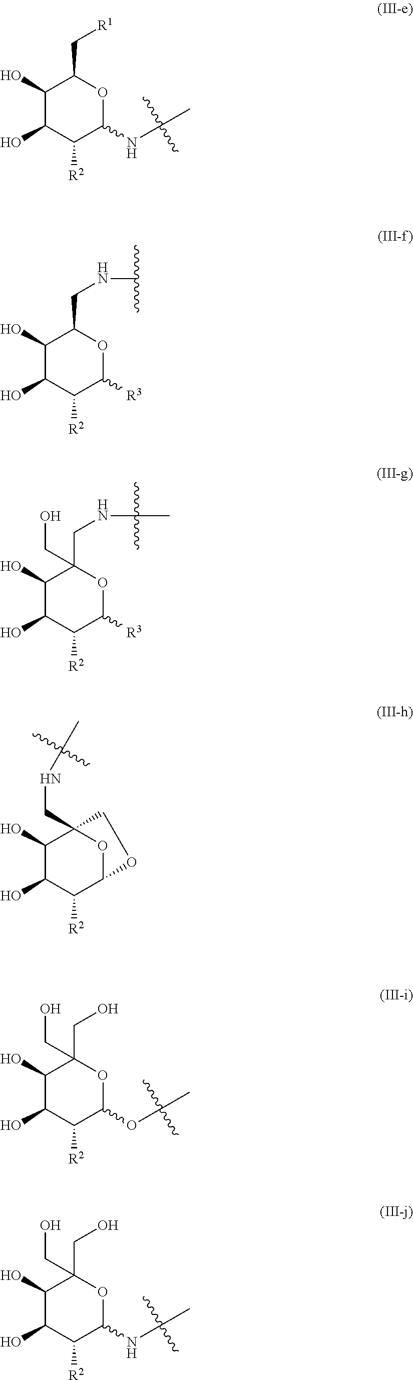

wherein:

R¹ is selected from —OH, —OC(O)R, and

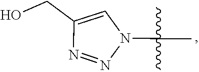

wherein R is C₁₋₆ alkyl;

R² is selected from —NHCOCH₃, —NHCOCF₃, —NHCOCH₂CF₃, —OH, and

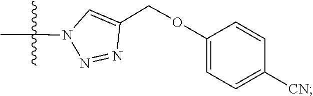 [SMILES: *n1cc(COc2ccc(C#N)cc2)nn1]

and

R³ is selected from —H, —OH, —CH₃, —OCH₃, and —OCH₂CH═CH₂.

Clause 107. The conjugate of clause 106, wherein X is:

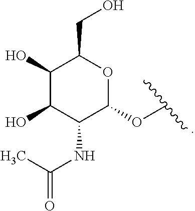 [SMILES: *O[C@H]1O[C@H](CO)[C@H](O)[C@H](O)[C@H]1NC(C)=O]

Clause 108. The conjugate of clause 106, wherein X is:

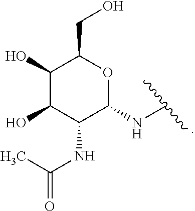 [SMILES: *N[C@H]1O[C@H](CO)[C@H](O)[C@H](O)[C@H]1NC(C)=O]

Clause 109. The conjugate of clauses 76 to 108, wherein the linker L is of formula (IIa):

-[(L¹)ₐ-(L²)_{b}-(L³)_{c}]ₙ-(L⁴)_{d}-(L⁵)ₑ-(L⁶)_{f}-(L⁷)_{g} (IIa)

wherein

each L¹ to L⁷ is independently a linking moiety and together provide a linear or branched linker between X and Y;

a is 1 or 2;

b, c, d, e, f, and g are each independently 0, 1, or 2;

n is 1 to 6 (e.g., n is 1 to 5, or 2 to 6, or 1, 2 or 3).

Clause 110. The conjugate of clause 109, wherein:

when d is O, n is 1;

when d is 1, n is 1 to 3; and

when d is 2, n is 1 to 6.

Clause 111. The conjugate of clause 109 or 110, wherein -(L¹)ₐ- comprises an optionally substituted aryl or heteroaryl linking moiety.

Clause 112. The conjugate of clause 111, wherein each L¹ is independently selected from

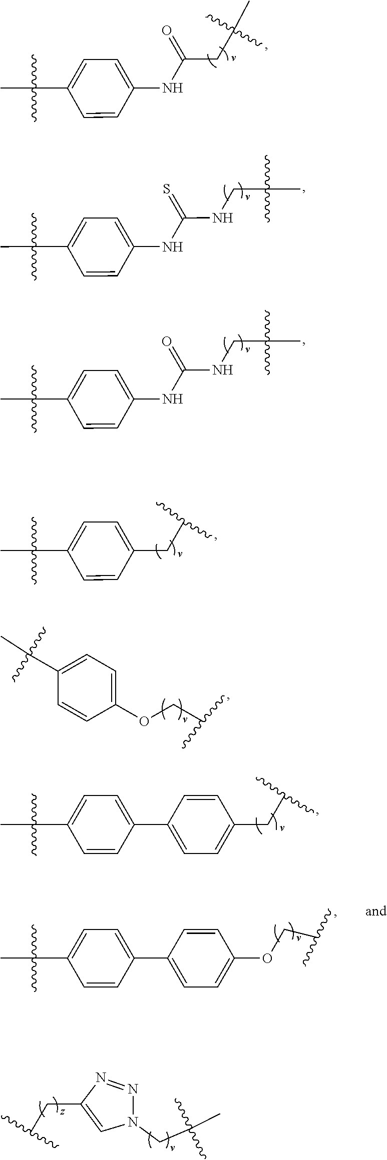

wherein v is 0 to 10 and z is 0 to 10.

Clause 113. The conjugate of any one of clauses 109 to 112, wherein:

each L² is independently selected from —C₁₋₆-alkylene-, —NHCO—C₁₋₆-alkylene-, —CONH—C₁₋₆-alkylene-, —O(CH₂)ₚ—, and —(OCH₂CH₂)ₚ—, wherein p is 1 to 10; and

each L³ is independently selected from:

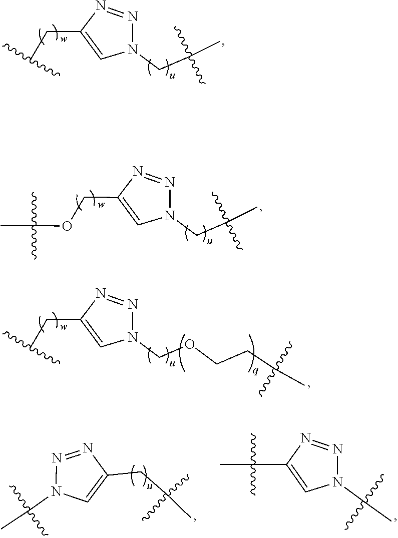

and —(OCH₂CH₂)_{q}—, wherein q is 1 to 10, u is 0 to 10, and w is 1 to 10. Clause 114. The conjugate of any one of clauses 109 to 113, wherein when n is 2 or more, at least one L⁴ is present and is a branched linking moiety.

Clause 115. The conjugate of any one of clauses 109 to 114, wherein each L⁴ is independently selected from:

—OCH₂CH₂—,

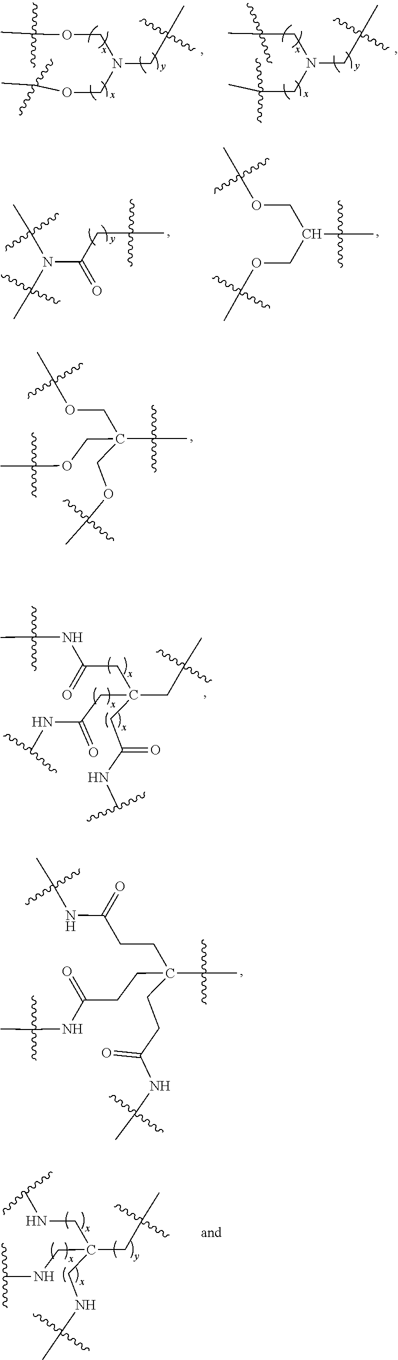

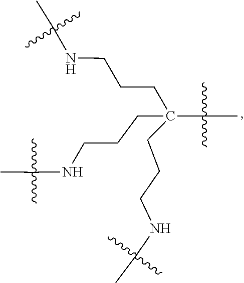

wherein each x and y are each independently 1 to 10.

Clause 116. The conjugate of any one of clauses 109 to 115, wherein:

each L⁵ is independently —NHCO—C₁₋₆-alkylene-, —CONH—C₁₋₆-alkylene-, —C₁₋₆-alkylene-,

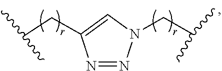

or —(OCH₂CH₂)ᵣ—;

each L⁶ is independently —NHCO—C₁₋₆-alkylene-, —CONH—C₁₋₆-alkylene-, —C₁₋₆-alkylene-, or —(OCH₂CH₂)ₛ—;

each L⁷ is independently —NHCO—C₁₋₆-alkylene-, —CONH—C₁₋₆-alkylene-, —C₁₋₆-alkylene-, —(OCH₂CH₂)ₜ—, or —OCH₂—; and

r, s, and t are each independently 1 to 20.

Clause 117. The conjugate of any one of clauses 109 to 116, wherein a is 1

Clause 118. The conjugate of any one of clauses 109 to 117, wherein at least one of b, c, e, f, and g is not 0.

Clause 119. The conjugate of any one of clauses 109 to 118, wherein at least one of b or c is not 0 and at least one of e, f, and g is not 0.

Clause 120. The conjugate of any one of clauses 109 to 119, wherein a, b, and c are each independently 1 or 2.

Clause 121. The conjugate of any one of clauses 109 to 120, wherein the linker L is selected from any one of the structures of Tables 2-3.

Clause 122. The conjugate of clause 76 or 77, wherein the conjugate is selected from:

ii) a conjugate derived from conjugation of a compound of any one of the structures of Tables 5-9 and a biomolecule;

iii) a conjugate derived from conjugation of a compound of any one of the structures of Table 5-9 and a polypeptide; or

iv) a conjugate derived from conjugation of a compound of any one of the structures of Table 5-9 and an antibody or antibody fragment.

Clause 123. The conjugate of any one of clauses 77-122, wherein the antibody or antibody fragment is an IgG antibody.

Clause 124. The conjugate of any one of clauses 77-122, wherein the antibody or antibody fragment is a humanized antibody.

Clause 125. The conjugate of any one of clauses 77-124, wherein the antibody or antibody fragment specifically binds to a secreted or soluble protein.

Clause 126. The conjugate of any one of clauses 77-124, wherein the antibody or antibody fragment specifically binds to a cell surface receptor.

Clause 127. A method of internalizing a target protein in a cell comprising a cell surface receptor selected from M6PR and ASGPR, the method comprising: contacting a cellular sample comprising the cell and the target protein with an effective amount of a compound according to any one of clauses 1 to 75, or a conjugate according to any one of clauses 76 to 132, wherein the compound or conjugate specifically binds the target protein and specifically binds the cell surface receptor to facilitate cellular uptake of the target protein.

Clause 128. The method of clause 127, wherein the target protein is a membrane bound protein.

Clause 129. The method of clause 127, wherein the target protein is an extracellular protein.

Clause 130. The method of any one of clauses 127 to 129, wherein the compound or conjugate comprises an antibody or antibody fragment (Ab) that specifically binds the target protein.

Clause 131. A method of reducing levels of a target protein in a biological system, the method comprising: contacting the biological system with an effective amount of a compound according to any one of clauses 1 to 75, or a conjugate according to any one of clauses 76 to 126, wherein the compound or conjugate specifically binds the target protein and specifically binds a cell surface receptor of cells in the biological system to facilitate cellular uptake and degradation of the target protein.

Clause 132. The method of clause 131, wherein the biological system comprises cells that comprise the cell surface receptor M6PR.

Clause 133. The method of clause 131, wherein the biological system comprises cells that comprise the cell surface receptor ASGPR.

Clause 134. The method of any one of clauses 131 to 133, wherein the biological system is a human subject.

Clause 135. The method of any one of clauses 131 to 133, wherein the biological system is an in vitro cellular sample.

Clause 136. The method of any one of clauses 131 to 135, wherein the target protein is a membrane bound protein.

Clause 137. The method of any one of clauses 137 to 135, wherein the target protein is an extracellular protein.

Clause 138. A method of treating a disease or disorder associated with a target protein, the method comprising: administering to a subject in need thereof an effective amount of a compound according to any one of clauses 1 to 75, or a conjugate according to any one of clauses 76 to 126, wherein the compound or conjugate specifically binds the target protein.

Clause 139. The method of clause 138, wherein the disease or disorder is an inflammatory disease.

Clause 140. The method of clause 138, wherein the disease or disorder is an autoimmune disease.

Clause 141. The method of clause 138, wherein the disease or disorder is a cancer.

Clause 151. A compound of the following formula (I):

Xₙ-L-Y (I);

or a salt, a single stereoisomer, a mixture of stereoisomers or an isotopic form thereof,
wherein:
X is a moiety that binds to a cell surface;
L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa); and

wherein
each L¹ is independently

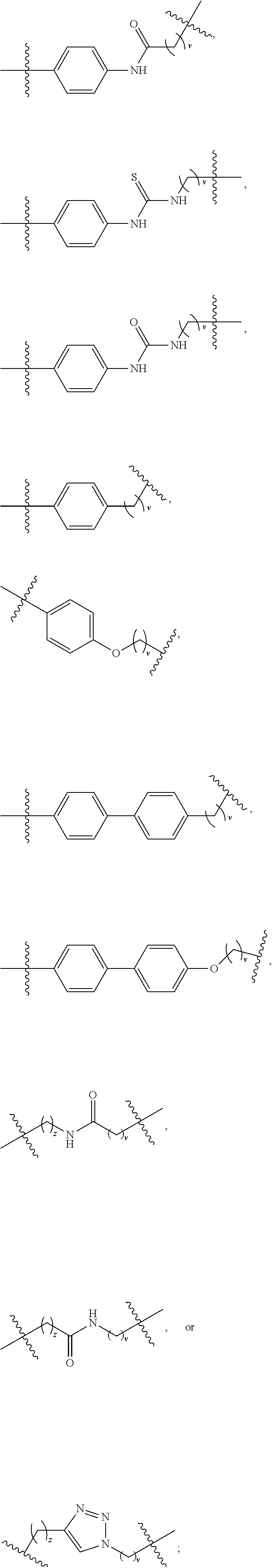

each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

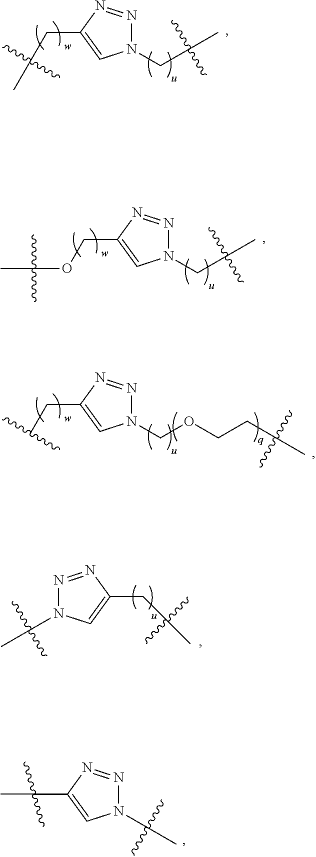

or —(OCH₂CH₂)_{q}—;
each L⁴ is independently —OCH₂CH₂—,

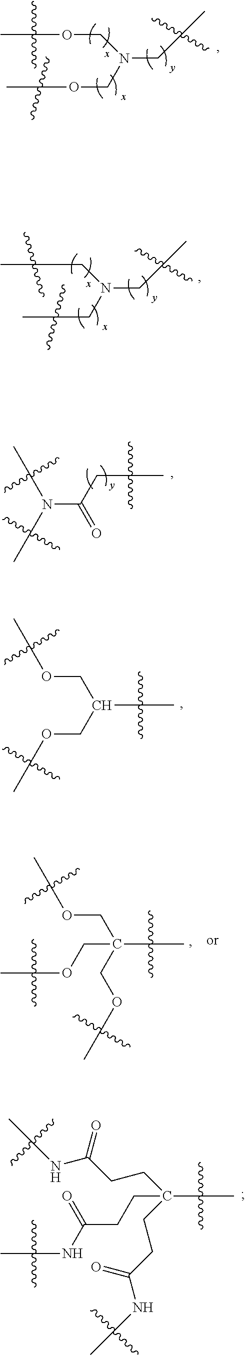

each L⁵ is independently —NHCO—C₁₋₆-alkylene-, —CONH—C₁₋₆-alkylene-, —C₁₋₆-alkylene-,

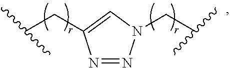

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—;
   p, q, r, s, and t are each independently an integer of 1 to 20; a is 1 or 2; b, c, d, e, f, and g are each independently 0, 1, or 2; u, v, w, x, y, and z are each independently an integer of 1 to 10;
   n is an integer of 1 to 5; wherein when d is O, n is 1, when d is 1, n is an integer of 1 to 3, and when d is 2, n is an integer of 1 to 5;
   Y is a moiety selected from the group consisting of

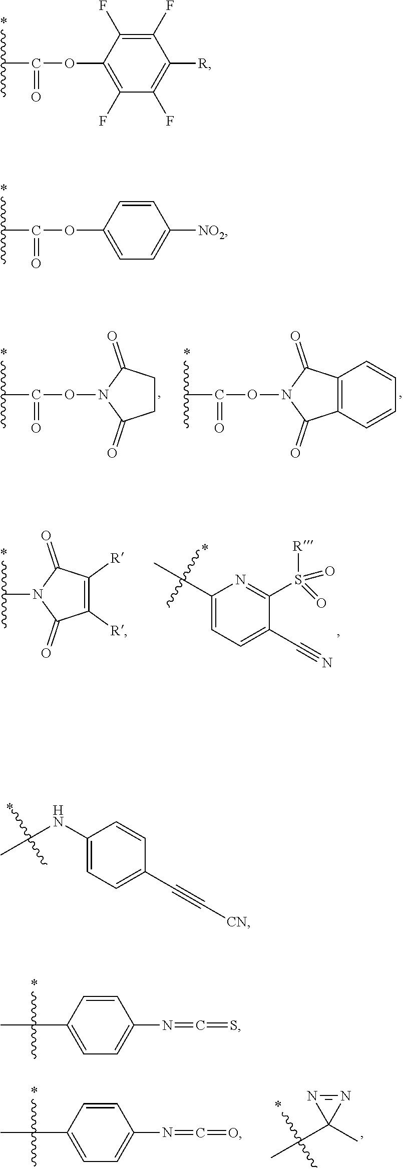

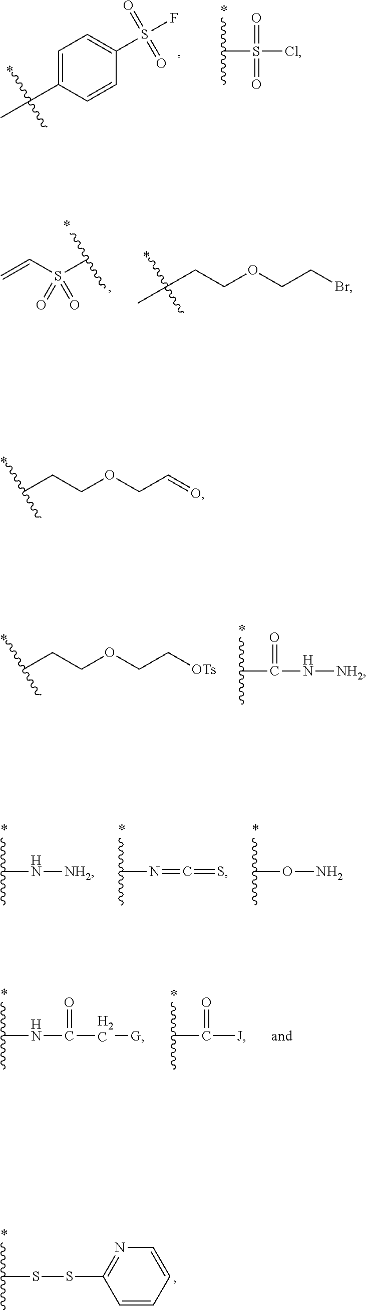

wherein [symbol] represents the point of attachment to L;
R is hydrogen or fluorine;
each R′ is independently hydrogen or halo;
G is selected from —F, —Cl, —Br, —I, —O-mesyl, and —O-tosyl;
J is selected from —Cl, —Br, —I, —F, —OH, —O—N-succinimide, —O-(4-nitrophenyl), —O-pentafluorophenyl, —O-tetrafluorophenyl, and —O—C(O)—OR^{J′}; and R^{J′} is —C₁-C₈ alkyl or aryl.

Clause 152. The compound of clause 151, wherein the cell surface receptor is a cell surface mannose-6-phosphate receptor (M6PR).

Clause 153. The compound of clause 151, wherein the cell surface receptor is a cell surface asialoglycoprotein receptor (ASGPR).

Clause 154. The compound of clause 151, wherein a is 1.

Clause 155. The compound of clause 151, wherein at least one of b, c, e, f, and g is not 0.

Clause 156. The compound of clause 151, wherein at least one of b or c is not 0 and at least one of e, f, and g is not 0.

Clause 157. The compound of clause 151, wherein a, b, and c are each independently 1 or 2.

Clause 158. The compound of clause 151, wherein each X is independently selected from the group consisting of formulas (IIIa), (IIIb), (IIIc), (IIId), (IIIj), (IIIk), (IIIl), and (IIIm):

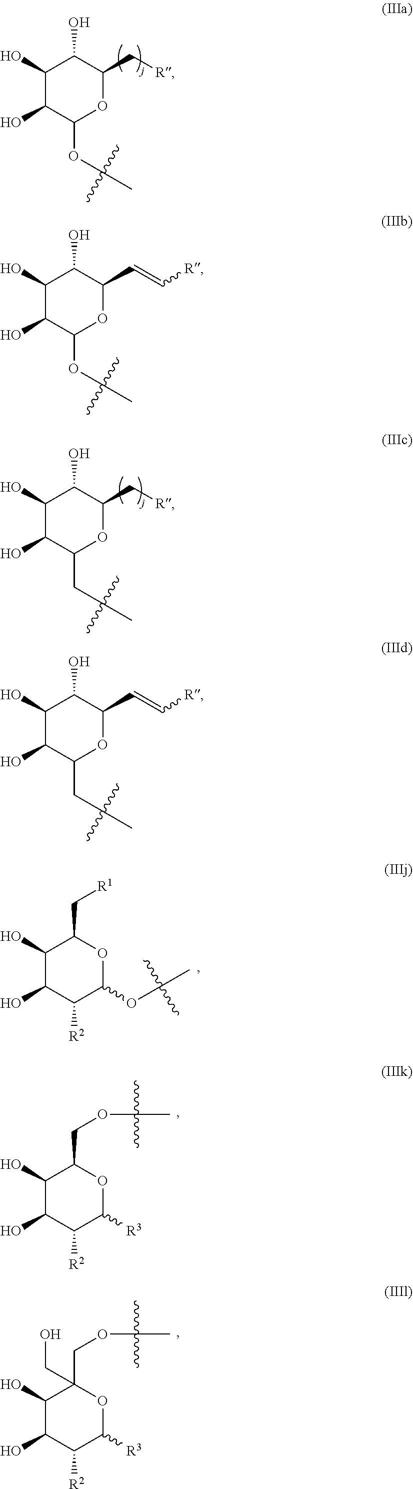

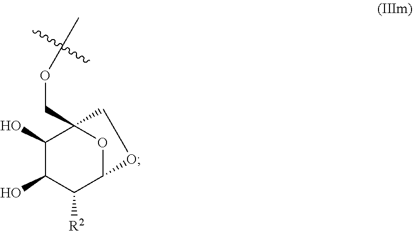

wherein in formula (IIIa), (IIIb), (IIIc), or (IIId):
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

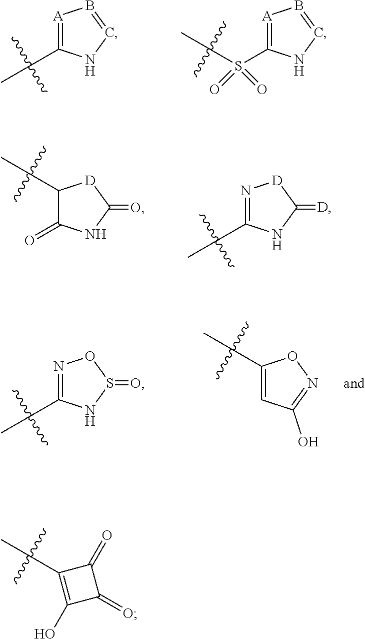

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S;
and
wherein in formula (IIIj), (IIIk), (IIIl), or (IIIm):

### R¹ is —OH, —OC(O)R, or

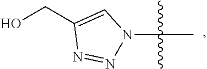

wherein R is C₁₋₆ alkyl;
R² is selected from the group consisting of —NHCOCH₃, —NHCOCF₃, —NHCOCH₂CF₃, —OH, and

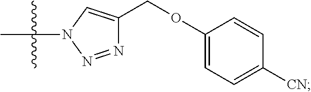 [SMILES: *n1cc(COc2ccc(C#N)cc2)nn1]

and
wherein R³ is selected from the group consisting of —H, —OH, —CH₃, —OCH₃, and —OCH₂CH═CH₂.

Clause 159. The compound of clause 151, wherein each X is independently selected from the group consisting of formulas (IIIa), (IIIb), (IIIc), and (IIId):

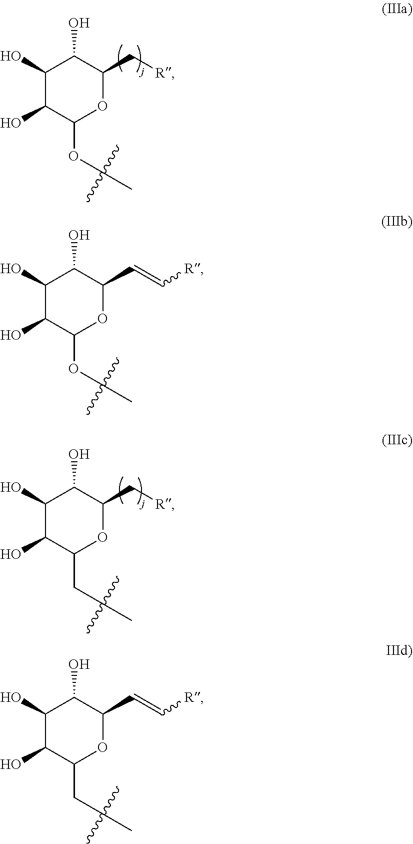

wherein
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

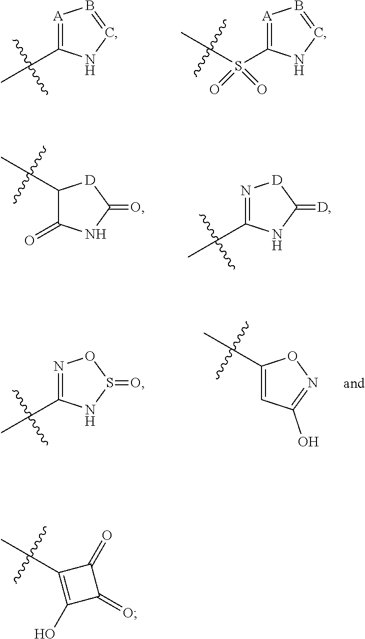

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S.

Clause 160. The compound of clause 151, wherein each X is independently selected from the group consisting of formulas (IIIj), (IIIk), (IIIl), and (IIIm):

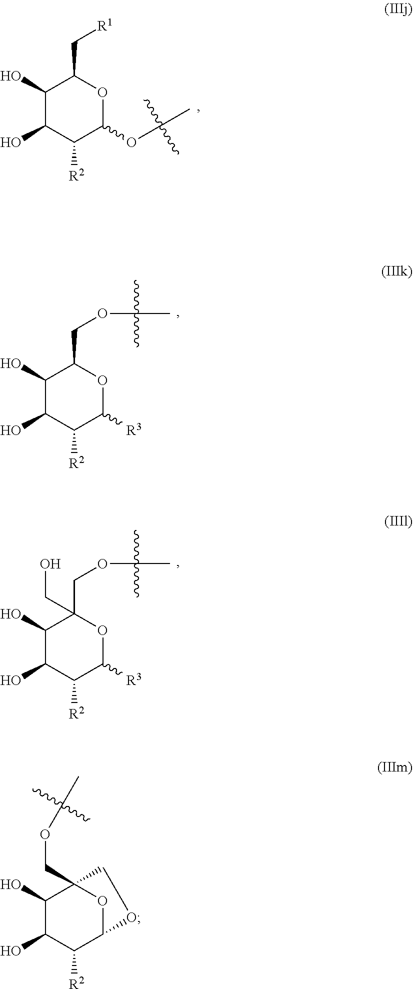

wherein

### R¹ is —OH, —OC(O)R, or

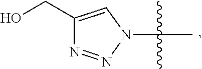

wherein R is C₁₋₆ alkyl;
R² is selected from the group consisting of —NHCOCH₃, —NHCOCF₃, —NHCOCH₂CF₃, —OH, and

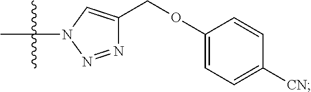 [SMILES: *n1cc(COc2ccc(C#N)cc2)nn1]

and
wherein R³ is selected from the group consisting of —H, —OH, —CH₃, —OCH₃, and —OCH₂CH═CH₂.

Clause 161. A conjugate of the following formula (IVa):

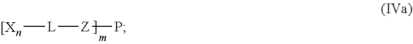

or a pharmaceutically acceptable salt thereof,
wherein:
X is a moiety that binds to a cell surface receptor;
L is a linker of the following formula (IIa):

-[(L¹)ₐ-(L²)_{b}-(L³)_{c}]ₙ-(L⁴)_{d}-(L⁵)ₑ-(L⁶)_{f}-(L⁷)_{g}- (IIa); and

wherein
each L¹ is independently

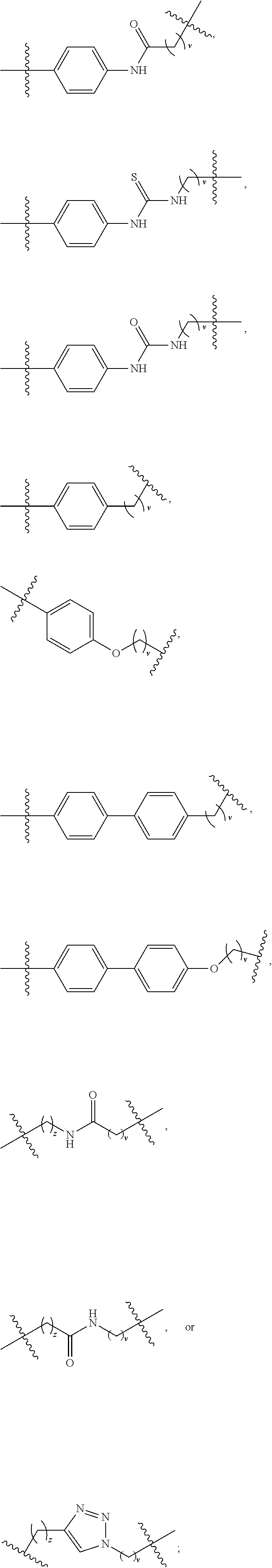

each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

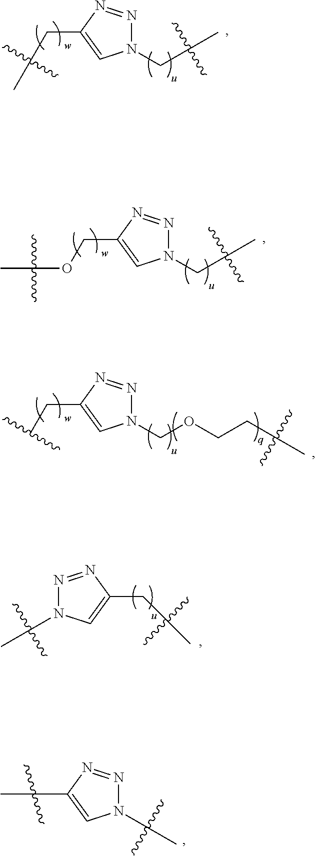

or —(OCH₂CH₂)_{q}—;
each L⁴ is independently —OCH₂CH₂—,

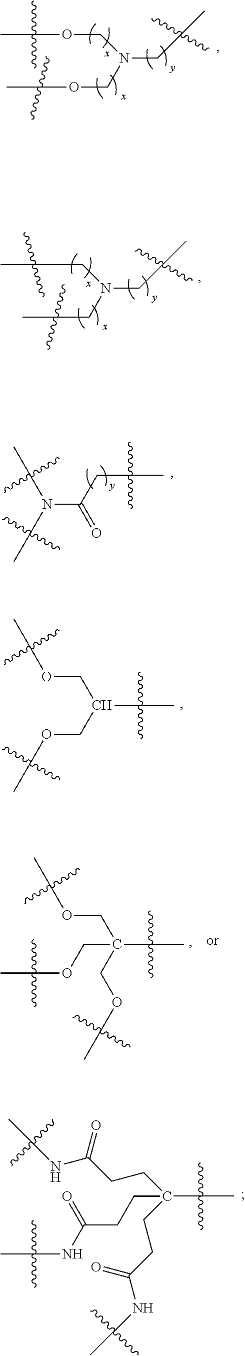

each L⁵ is independently —NHCO—C₁₋₆-alkylene-, —CONH—C₁₋₆-alkylene-, —C₁₋₆-alkylene-,

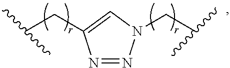

or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C1-6-alkylene-, C₁₋₆-alkylene-, —(OCH₂CH₂)ₜ—, or —OCH₂—;
   p, q, r, s, and t are each independently an integer of 1 to 20; a is 1 or 2; b, c, d, e, f, and g are each independently 0, 1, or 2; u, v, w, x, y, and z are each independently an integer of 1 to 10;
   n is an integer of 1 to 5; wherein when d is O, n is 1, when d is 1, n is an integer of 1 to 3, and when d is 2, n is an integer of 1 to 5;
   Z is selected from the group consisting of

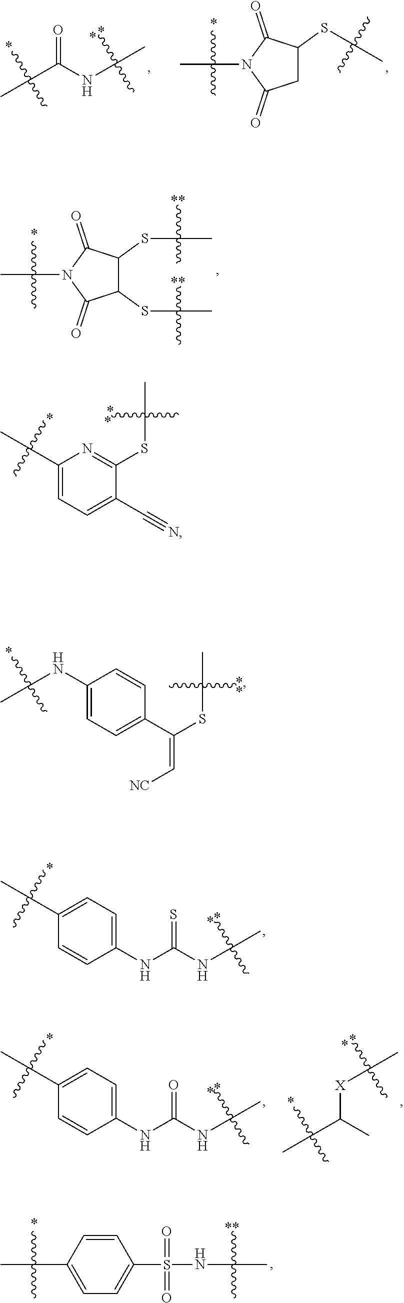

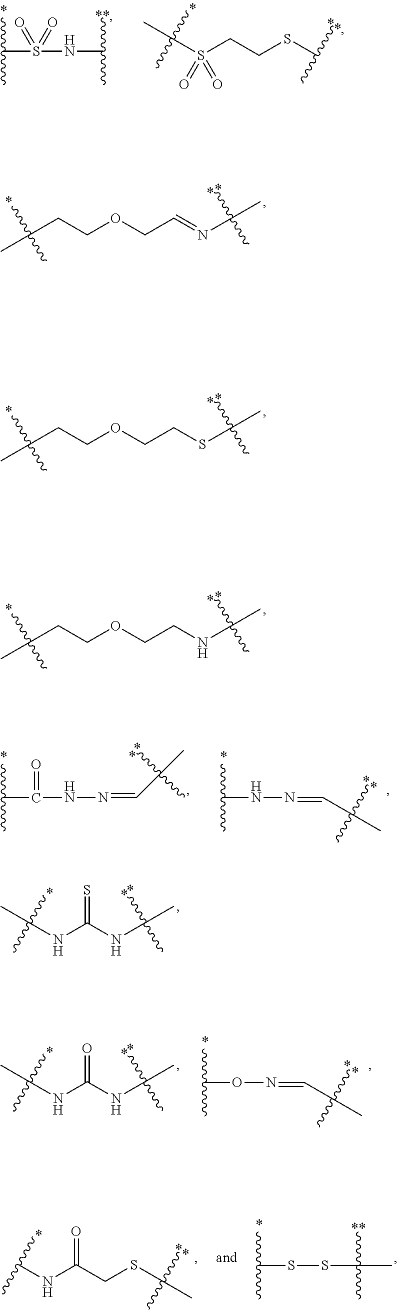

wherein [symbol] represents the point of attachment to L,
wherein [symbol] represents the point of attachment to P,

### X is CH₂, NH, O or S; and

P is a polypeptide.

Clause 162. The conjugate of clause 161, wherein P comprises an antibody or an antigen-binding fragment of an antibody.

Clause 163. A conjugate of the following formula (Va):

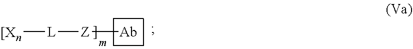

or a pharmaceutically acceptable salt thereof,
wherein:
X is a moiety that binds to a cell surface receptor;
L is a linker of the following formula (IIa):

-[(L¹)ₐ-(L²)_{b}-(L³)_{c}]ₙ-(L⁴)_{d}-(L⁵)ₑ-(L⁷)_{g}- (IIa); and

wherein
each L¹ is independently

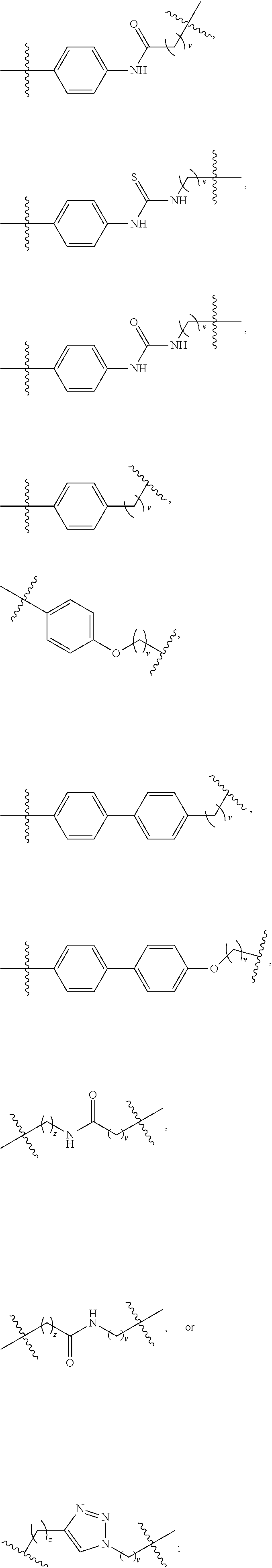

each L² is independently —C₁₋₆-alkylene-, —NHCO—C₁₋₆-alkylene-, —CONH—C₁₋₆-alkylene-, —(OCH₂)ₚ—, or —(OCH₂CH₂)ₚ—;
each L³ is independently

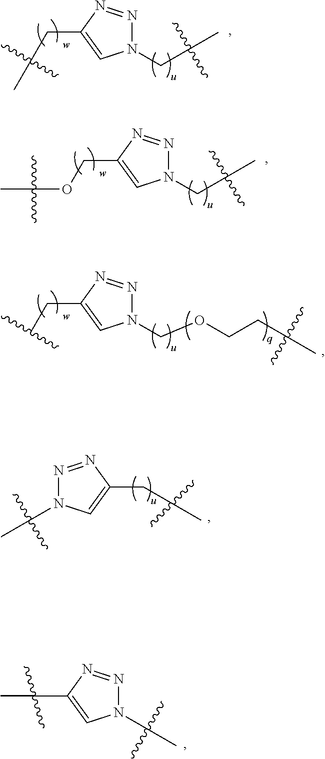

or —(OCH₂CH₂)_{q}—; each L⁴ is independently —OCH₂CH₂—,

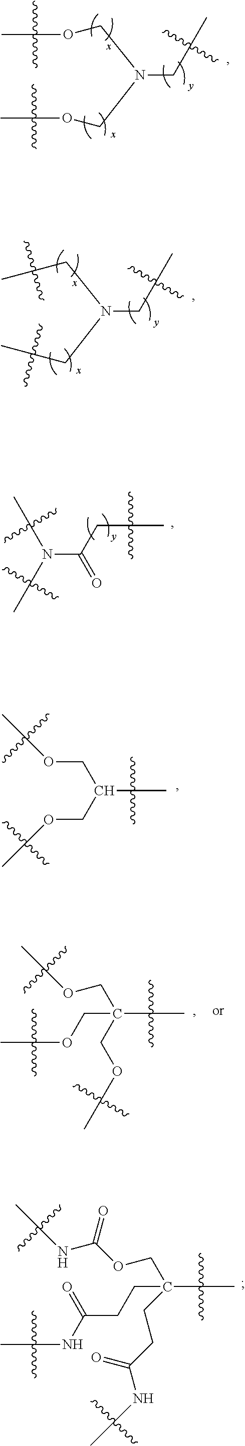

each L⁵ is —NHCO—C₁₋₆-alkylene-, —CONH—C₁₋₆-alkylene-, —C₁₋₆-alkylene-,

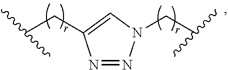

or —(OCH₂CH₂)ᵣ—;
each L⁶ is —NHCO—C₁₋₆-alkylene-, —CONH—C₁₋₆-alkylene-, —C₁₋₆-alkylene-, or —(OCH₂CH₂)ₛ—;
each L⁷ is —NHCO—C₁₋₆-alkylene-, —CONH—C₁₋₆-alkylene-, —C₁₋₆-alkylene-, —(OCH₂CH₂)ₜ—, or —OCH₂—;

p, q, r, s, and t are each independently an integer of 1 to 20; a is 1 or 2; b, c, d, e, f, and g are each independently 0, 1, or 2; u, v, w, x, y, and z are each independently 1, 2, 3, 4, 5, or 6;

n is an integer of 1 to 5; wherein when d is O, n is 1, when d is 1, n is an integer of 1 to 3, and when d is 2, n is an integer of 1 to 5;

m is an integer from 1 to 8;

Z is selected from the group consisting of

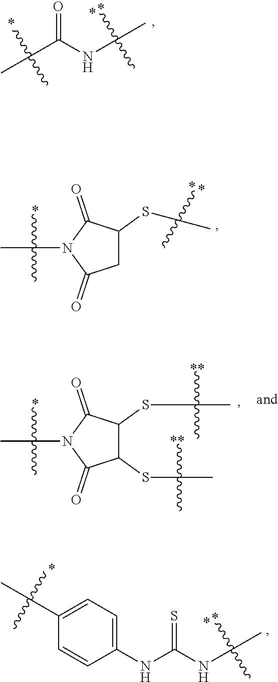

wherein [symbol] represents the point of attachment to L, wherein [symbol] represents the point of attachment to

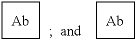

is an antibody.

Clause 164. The conjugate of any one of clauses 161-163, wherein the cell surface receptor is a cell surface mannose-6-phosphate receptor (M6PR).

Clause 165. The conjugate of any one of clauses 161-163, wherein the cell surface receptor is a cell surface asialoglycoprotein receptor (ASGPR).

Clause 166. The conjugate of any one of clauses 161-165, wherein each X is independently selected from the group consisting of formulas (IIIa), (IIIb), (IIIc), (IIId), (IIIj), (IIIk), (IIIl), and (IIIm):

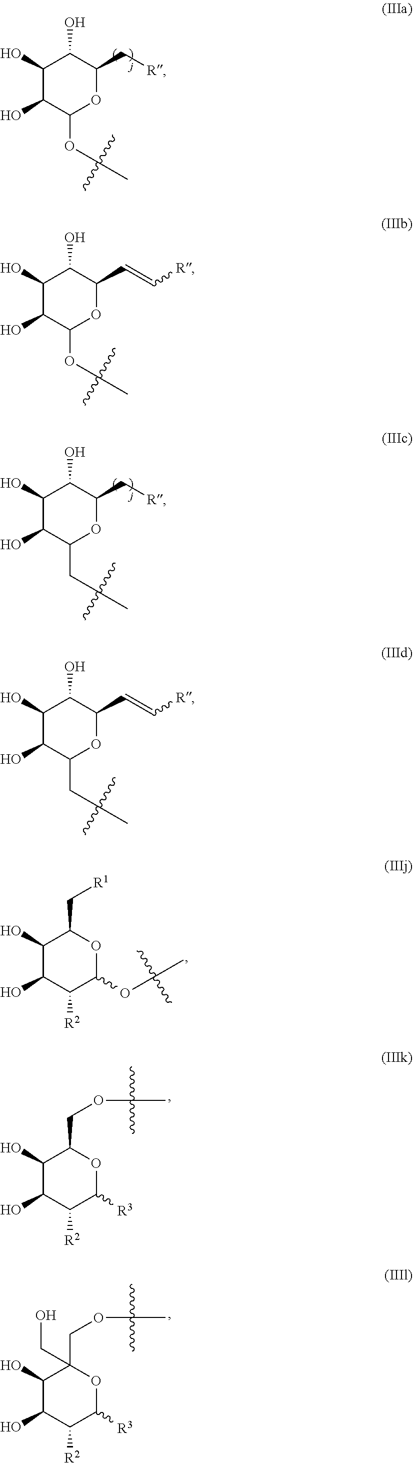

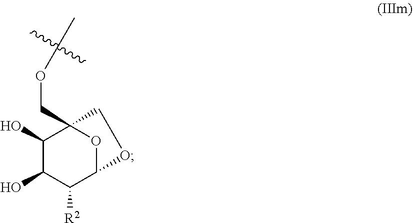

wherein in formula (IIIa), (IIIb), (IIIc), or (IIId):
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)NHS(O)₂R³, —NHSO₂R³,

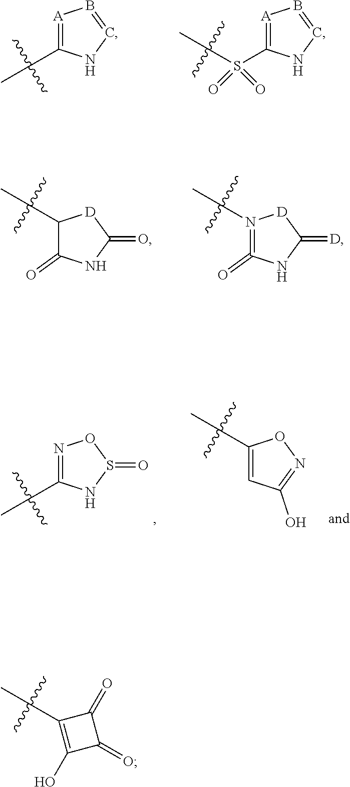

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S;
and
wherein in formula (IIIj), (IIIk), (IIIl), or (IIIm):

R¹ is —OH, —OC(O)R, or

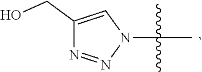

wherein R is C₁₋₆ alkyl;
R² is selected from the group consisting of —NHCOCH₃, —NHCOCF₃, —NHCOCH₂CF₃, —OH, and

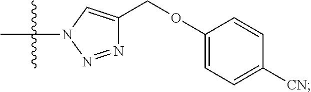 [SMILES: *n1cc(COc2ccc(C#N)cc2)nn1]

and
wherein R³ is selected from the group consisting of —H, —OH, —CH₃, —OCH₃, and —OCH₂CH═CH₂.

Clause 167. The conjugate of any one of clauses 161-165, wherein each X is independently selected from the group consisting of formulas (IIIa), (IIIb), (IIIc), and (IIId):

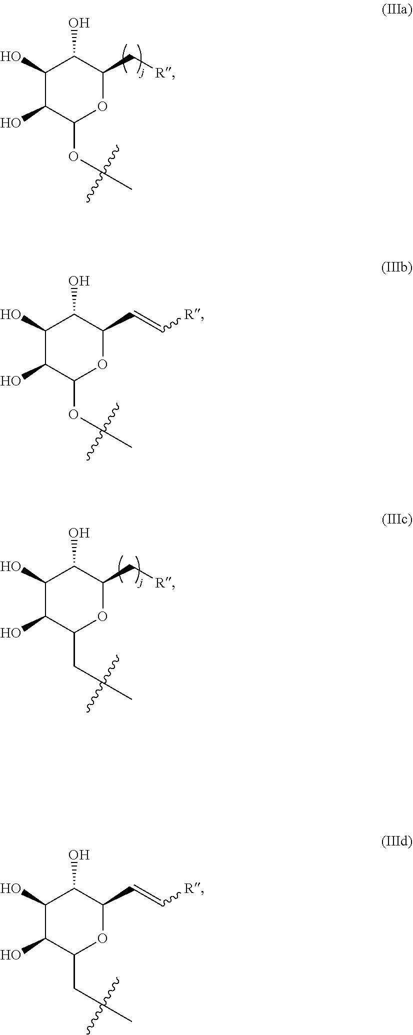

wherein
R″ is selected from the group consisting of —OH, —CR¹R²OH, —P═O(OH)₂, P(═O)R¹OH, —PH(═O)OH, —(CR¹R²)—P═O(OH)₂, —SO₂OH, —S(O)OH, —OSO₂OH, —COOH, —CONH₂, —CONHR³, —CONR³R⁴, —CONH(OH), —CONH(OR³)—CONHSO₂R³, —CONHSO₂NR³R⁴, —CH(COOH)₂, —CR¹R²COOH, —SO₂R³, —SOR³R⁴, —SO₂NH₂, —SO₂N H R³, —SO₂NR³R⁴, —SO₂NHCOR³, —NHCOR³, —NHC(O)NHS(O)₂R³, —NHSO₂R³,

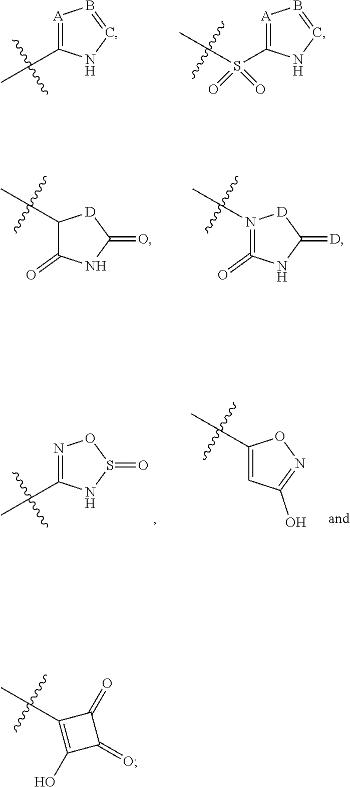

j is an integer of 1 to 3;
R¹ and R² are each independently hydrogen, halo, or CN;
R³ and R⁴ are each independently C₁₋₆ alkyl;
A, B, and C are each independently CH or N;
D is each independently O or S.

Clause 168. The conjugate of any one of clauses 161-165, wherein each X is independently selected from the group consisting of formulas (IIIj), (IIIk), (IIIl), and (IIIm):

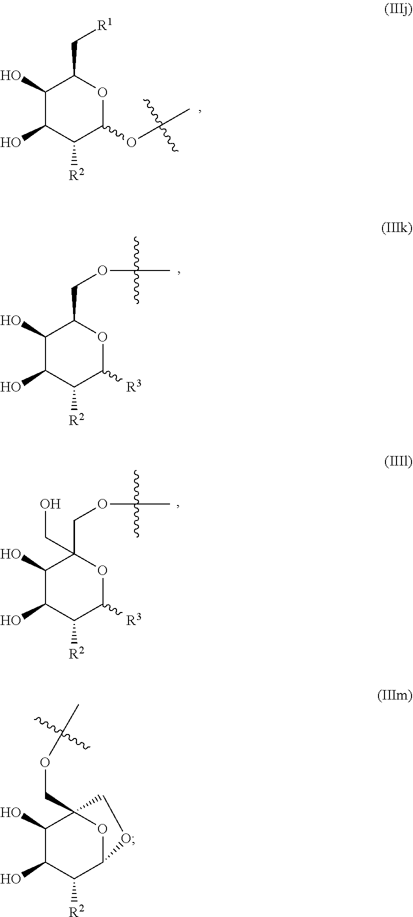

wherein

### R¹ is —OH, —OC(O)R, or

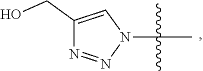

wherein R is C₁₋₆ alkyl;
R² is selected from the group consisting of —NHCOCH₃, —NHCOCF₃, —NHCOCH₂CF₃, —OH, and

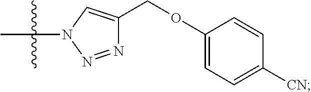 [SMILES: *n1cc(COc2ccc(C#N)cc2)nn1]

and
wherein R³ is selected from the group consisting of —H, —OH, —CH₃, —OCH₃, and —OCH₂CH═CH₂.

Clause 169. A pharmaceutical composition comprising the conjugate or pharmaceutically acceptable salt of any one of clauses 161-168, and a pharmaceutically acceptable carrier.

Clause 170. The pharmaceutical composition of clause 169, wherein m is an integer of 4 to 8.

Clause 171. The pharmaceutical composition comprising the conjugate or pharmaceutically acceptable salt of clause 170, wherein m is 4.

Clause 172. The conjugate of any one of clauses 163-168, wherein the antibody is an IgG antibody.

Clause 173. The conjugate of any one of clauses 163-168, wherein the antibody is a humanized antibody.

Clause 174. The conjugate of any one of clauses 163-168, wherein the antibody specifically binds to a secreted or soluble protein.

Clause 175. The conjugate of any one of clauses 163-168, wherein the antibody specifically binds to a cell surface receptor.

Clause 176. The conjugate of any one of clauses 163-168, wherein the antibody specifically binds to programmed death ligand-1 (PD-L1) protein.

Clause 177. The conjugate of any one of clauses 163-168, wherein the antibody specifically binds to Vascular Endothelial Growth Factor (VEGF) protein.

Clause 178. The conjugate of any one of clauses 163-168, wherein the antibody specifically binds to a Fibroblast Growth Factor Receptor 2 (FGFR2) protein or a Fibroblast Growth Factor Receptor 3 (FGFR3) protein.

Clause 179. The conjugate of any one of clauses 163-168, wherein the antibody is cetuximab.

Clause 180. The conjugate of any one of clauses 163-168, wherein the antibody is matuzumab.

Clause 181. The conjugate of any one of clauses 163-168, wherein the antibody is atezolizumab.

Clause 182. A method of treating a disease or disorder by administering to a subject in need thereof an effective amount of the conjugate or pharmaceutically acceptable salt of any one of clauses 163-168 or the pharmaceutical composition of clause 169.

Clause 183. The method of clause 182, wherein the disease or disorder is an inflammatory disease.

Clause 184. The method of clause 182, wherein the disease or disorder is an autoimmune disease.

Clause 185. The method of clause 182, wherein the disease or disorder is a cancer.

### EXAMPLES

The examples in this section are offered by way of illustration, and not by way of limitation.

### Abbreviations/Acronyms

| Acronym/ | |
|---|---|
| Abbreviation | Meaning |
| Ab | antibody |
| AF647 | Alexa Fluor 647 |
| BCA | bicinchoninic acid |
| BMPS | 3-Maleimidopropionic acid N-hydroxysuccinimide ester |
| BSA | bovine serum albumin |
| Ctx | cetuximab |
| DAPI | 4′,6-diamidino-2-phenylindole |
| DAR | drug-to-antibody ratio |
| DBU | 1,8-diazabicyclo(5.4.0)undec-7-ene |
| DCC | N,N′-dicyclohexylcarbodiimide |
| DCM | dichloromethane |
| DMA | N,N-dimethyl acetamide |
| DMEM | Dulbecco's modified eagle's medium |
| DMF | N,N-dimethyl formamide |
| DMSO | dimethyl sulfoxide |
| DOL | degree of labeling |
| DTNB | 5,5′-dithiobis-(2-nitrobenzoic acid) |
| EDTA | ethylenediaminetetraacetic acid |
| EGFR | epidermal growth factor receptor |
| EMCS | 6-Maleimidocaproic acid N-succinimidyl ester |
| FACS | flow cytometry staining |
| FBS | fetal bovine serum |
| HBVS | 1,6-Hexane bis-vinylsulfone |
| HEPES | (4-(2-hydroxyethyl)-1-piperazineethanesulfonic acid) |
| HIC | hydrophobic interaction chromatography |
| HPLC | high performance liquid chromatography |
| GMBS | 4-Maleimidobutyric acid N-hydroxysuccinimide ester |
| IgG | immunoglobulin G |
| KO | knock-out |
| LC-SMCC | Succinimidyl-4-(N-maleimidomethyl)cyclohexane-1- |
| | carboxylate |
| M6P | mannose-6-phosphate |
| M6PR | mannose-6-phosphate receptor |
| MBS | m-Maleimidobenzoyl-N-hydroxysuccinimide ester |
| MFI | mean fluorescence intensity |
| MPBH | 4-(4-N-Maleimidophenyl)butyric acid hydrazide |
| | hydrochloride |
| MS | mass spectrometry |
| Mtz | matuzumab |
| NMP | N-methyl-2-pyrrolidone |
| PBS | phosphate-buffered saline |
| PEG | Polyethylene glycol |
| PFA | paraformaldehyde |
| RIPA | radioimmunoprecipitation assay |
| RT | room temperature |
| SBAP | Succinimidyl 3-(bromoacetamido)propionate |
| SEC | size exclusion chromatography |
| SIA | Succinimidyl iodoacetate |
| SIAB | Succinimidyl (4-iodoacetyl)aminobenzoate |
| SMCC | Succinimidyl-4-(N-maleimidomethyl)cyclohexane-1- |
| | carboxylate |
| SMPB | Succinimidyl 4-(p-maleimidophenyl)butyrate |
| SMPH | Succinimidyl 6-((beta-maleimidopropionamido)hexanoate) |
| Sulfo-EMCS | N-ε-maleimidocaproyl-oxysulfosuccinimide ester |
| Sulfo-GMBS | N-γ-maleimidobutyryl-oxysulfosuccinimide ester |
| Sulfo-KMUS | N-κ-maleimidoundecanoyl-oxysulfosuccinimide ester |
| Sulfo-MBS | m-maleimidobenzoyl-N-hydroxysulfosuccinimide ester |
| Sulfo-SIAB | sulfosuccinimidyl (4-iodoacetyl)aminobenzoate |
| Sulfo-SMCC | 4-(N-maleimidomethyl)cyclohexane-1-carboxylic 3- |
| | sulfohydroxysuccinimide ester |
| Sulfo-SMPB | sulfosuccinimidyl 4-(N-maleimidophenyl)butyrate |
| SVSB | succinimidyl-(4-vinylsulfone)benzoate |
| TFA | Trifluoroacetic acid |
| THF | tetrahydrofuran |
| TCEP | tris(2 carboxyethyl)phosphine |
| Ts | Tosyl |
| UPLC | ultra-performance liquid chromatography |
| w/v | weight by volume |

### Preparation of Compounds

The following are illustrative schemes and examples of how the compounds described herein can be prepared and tested. Although the examples can represent only some embodiments, it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

### Mannose-6-Phosphate (M6P) Ligands

### Compound A. Synthesis of (2-((2R,3S,4S,5S,6R)-3,4,5-trihydroxy-6-(4-isothiocyanatophenoxy)tetrahydro-2H-pyran-2-yl)ethyl)phosphonic acid (Compound A)

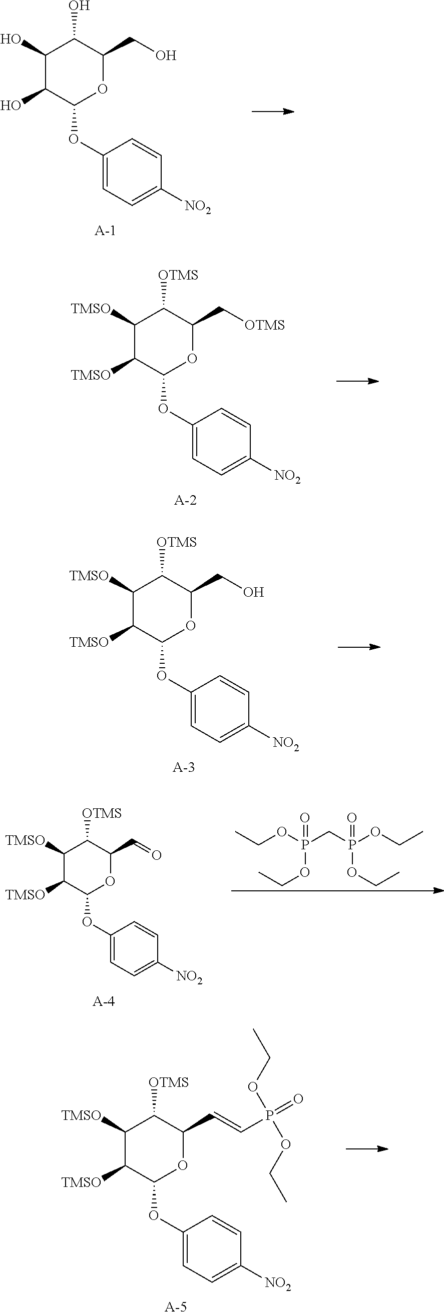

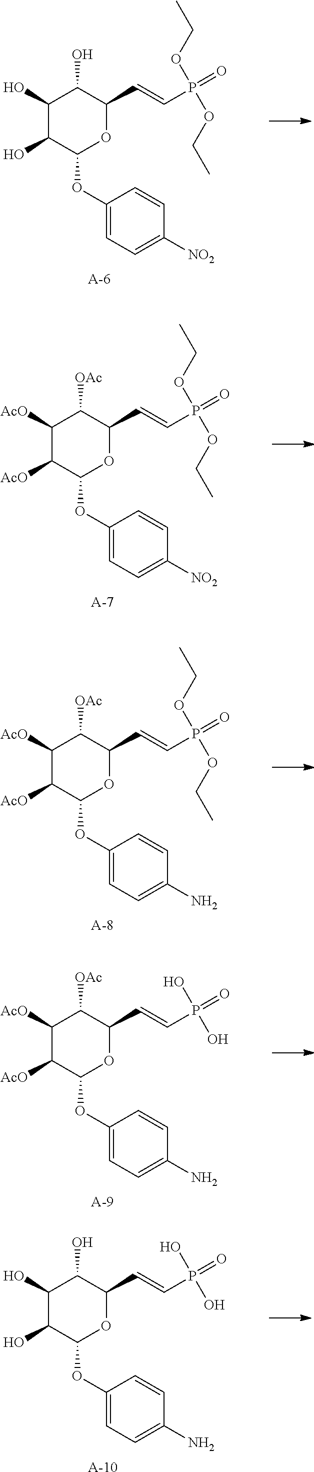

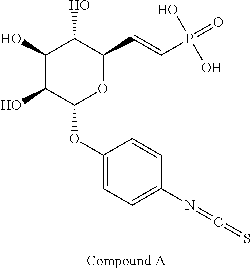 [SMILES: O=P(O)(O)/C=C/[C@H]1O[C@H](Oc2ccc(N=C=S)cc2)[C@@H](O)[C@@H](O)[C@@H]1O ; "Compound A"]

### (((2R,3S,4S,5R,6R)-2-(4-nitrophenoxy)-6(((trimethylsilyl)oxy)methyl)tetrahydro-2H-pyran-3,4,5-triyl)tris(oxy))tris(trimethylsilane) (A-2)

A solution of (2R,3S,4S,5S,6R)-2-(hydroxymethyl)-6-(4-nitrophenoxy)tetrahydro-2H-pyran-3,4,5-triol (A-1) (1.0 eq, 26.0 g, 86.37 mmol) in DMF (500 mL) was cooled to 0° C. Then triethylamine (6.4 eq, 288 mL, 552.0 mmol) and trimethylsilyl chloride (24.0 eq 70 mL, 2071.0 mmol) were added under nitrogen atmosphere to above solution. The resulting mixture was stirred at room temperature under nitrogen for 24 h. The reaction mixture was partitioned between ethyl acetate and water. The water layer was extracted again with ethyl acetate. The combined organic layers were dried over sodium sulfate, filtered, and purified via silica gel chromatography (0 to 5% ethyl acetate in hexane) to afford Intermediate A-2 as colorless oil. Yield: 36.8 g (72.3%); ¹H NMR (400 MHz, CDCl₃) δ 8.18 (dd, J=12.36, 3.16 Hz, 2H), 7.16 (dd, J=12.4, 3.12 Hz, 2H), 5.37 (d, J=2.36 Hz, 1H), 3.99-3.87 (m, 3H), 3.72-3.69 (m, 2H), 3.50-3.48 (m, 1H), 0.2-0.07 (m, 36H).

### ((2R,3R,4S,5S,6R)-6-(4-nitrophenoxy)-3,4,5-tris((trimethylsilyl)oxy)tetrahydro-2H-pyran-2-yl)methanol (A-3)

To a stirred solution of Intermediate A-2 (1.0 eq, 10.0 g, 16.97 mmol) in mixture of DCM:methanol (8:2 ratio, 100 mL) ammonium acetate (1.5 eq, 1.96 g, 25.46 mmol) was added at room temperature under nitrogen. The resulting mixture was stirred at room temperature under nitrogen for 16 h. The reaction mixture was partitioned between ethyl acetate and water. The water layer was extracted again with ethyl acetate. The combined organic layers were dried over sodium sulfate, filtered, concentrated under vacuum and purified via silica gel chromatography (20-30% ethyl acetate in hexane) to afford Intermediate A-3 as white solid. Yield: 7.0 g (80%); LC-MS m/z 516.13 [M−1]⁻.

### (2S,3R,4S,5S,6R)-6-(4-nitrophenoxy)-3,4,5-tris((trimethylsilyl)oxy)tetrahydro-2H-pyran-2-carbaldehyde (A-4)

To a stirred solution of oxalyl chloride (1.1 eq, 0.5 mL, 5.31 mmol) in DCM (5 mL) at −78° C. was added a solution of DMSO (2.2 eq, 0.76 mL, 10.62 mmol) in DCM (5 mL) over 5 min. After being stirred at −78° C. for 20 min, a solution of Intermediate A-3 (1.0 eq, 2.5 g, 4.83 mmol) in DCM (10 mL) was added to the mixture. The reaction mixture was further stirred at −78° C. for 60 min, followed by addition of triethylamine (5.0 eq, 3.4 mL, 24.15 mmol). The resulting mixture was allowed to reach room temperature over 1 h. The turbid mixture was diluted with DCM and washed with water followed by brine solution. The organic layer was dried over sodium sulfate, filtered, and concentrated under high vacuum to afford Intermediate A-4 as light brown gel (2.2 g, crude), which was used without further purification for the next step.

### Diethyl ((E)-2-((2R,3R,4S,5S,6R)-6-(4-nitrophenoxy)-3,4,5-tris((trimethylsilyl)oxy)tetrahydro-2H-pyran-2-yl)vinyl)phosphonate (A-5)

A stirred suspension of tetraethyl methylenebis(phosphonate) (1.5 eq, 1.85 g, 6.40 mmol) in dry THF (20 mL) was cooled to −78° C. and added n-BuLi in hexane 2.0 M (1.25 eq, 2.6 ml, 5.33 mmol). The resulting mixture was stirred for 1 h at −78° C., then Intermediate A-4 (1.0 eq, 2.2 g, 4.27 mmol) in dry THF (10 mL) was added at −78° C. The bath was removed and the reaction mixture was allowed to room temperature and stirring continued for 12 h. A saturated aqueous solution of NH₄Cl was added and extracted with ethyl acetate. Ethyl acetate layer washed with water followed by saturated brine solution. The organic layer was dried over sodium sulfate, filtered and concentrated. The crude was purified via silica gel chromatography (30-40% ethyl acetate in hexane) to afford Intermediate A-5 as colorless gel. Yield (1.3 g, 48%); LC-MS m/z 650.57 [M+1]⁺.

### Diethyl ((E)-2-((2R,3S,4S,5S,6R)-3,4,5-trihydroxy-6-(4-nitrophenoxy)tetrahydro-2H-pyran-2-yl)vinyl)phosphonate (A-6)

To a stirred solution of Intermediate A-5 (1.0 eq, 1.3 g, 1.54 mmol) in methanol (15 mL). was added Dowex 50WX8 hydrogen form at room temperature under nitrogen atmosphere. The resulting mixture was stirred at room temperature under nitrogen for 2 h. The reaction mixture filtered and washed with methanol, filtrate concentrated under vacuum to afford diethyl ((E)-2-((2R,3S,4S,5S,6R)-3,4,5-trihydroxy-6-(4-nitrophenoxy)tetrahydro-2H-pyran-2-yl)vinyl)phosphonate (6) as white solid. Yield: 0.78 g (90%); LC-MS m/z 434.17 [M+1]⁺.

### (2R,3R,4S,5S,6R)-2-((E)-2-(diethoxyphosphoryl)vinyl)-6-(4-nitrophenoxy)tetrahydro-2H-pyran-3,4,5-triyl triacetate (A-7)

To a stirred solution of Intermediate A-6, (1.00 eq, 0.78 g, 1.80 mmol) in pyridine (10 mL) was added an acetic anhydride (10.0 eq, 1.8 mL, 18.0 mmol) dropwise at 0° C. under nitrogen. The cold bath was removed and the resulting mixture was stirred at room temperature under nitrogen for 16 h. Pyridine was removed on a high vacuum and the residue was partitioned between ethyl acetate and aqueous 1N HCl. The water layer was extracted again with ethyl acetate. The combined organic layers were dried over sodium sulfate, filtered, concentrated and purified via silica gel chromatography (2.5% methanol in dichloromethane) to afford Intermediate A-7 as white solid. Yield: 1.0 g (100%); LC-MS m/z 560.17 [M+1]⁺.

### (2R,3S,4S,5R,6R)-2-(4-aminophenoxy)-6-(2-(diethoxyphosphoryl)ethyl)tetrahydro-2H-pyran-3,4,5-triyl triacetate (A-8)

To a stirred solution of Intermediate A-7 (1.0 eq, 1.0 g, 1.78 mmol) in methanol (15 mL) 10% palladium on carbon (0.200 g) was added at room temperature under nitrogen. The resulting mixture was stirred at room temperature under hydrogen gas pressure (100 psi) for 16 h. The reaction mixture filtered through Celite bed and washed with methanol, filtrate concentrated under vacuum to afford Intermediate A-8 as brown sticky gel. Yield: 0.700 g (73.6%); LC-MS m/z 532.21 [M+1]⁺.

### (2-((2R,3R,4S,5S,6R)-3,4,5-triacetoxy-6-(4-aminophenoxy)tetrahydro-2H-pyran-2-yl)ethyl)phosphonic acid (A-9)

To a stirred solution of Intermediate A-8 (1.00 eq, 2.0 g, 5.73 mmol) in acetonitrile (15 mL) bromotrimethylsilane (5.0 eq, 3.8 mL, 28.65 mmol) was added dropwise at 0° C. under nitrogen. The cold bath removed and the resulting mixture was stirred at room temperature under nitrogen for 16 h. Volatiles were removed on a rotary evaporator and the residue was dried under high vacuum. The crude residue was triturated with diethyl ether and dried under high vacuum to afford Intermediate A-9 as brown solid. Yield: 2.2 g, crude. LC-MS m/z 476.0 [M+1]⁺.

### (2-((2R,3S,4S,5S,6R)-6-(4-aminophenoxy)-3,4,5-trihydroxytetrahydro-2H-pyran-2-yl)ethyl)phosphonic acid (A-10)

To a stirred solution of Intermediate A-9 (1.0 eq, 2.0 g, 4.21 mmol) in mixture of methanol:water (8:2, 15 mL) triethylamine (5.0 eq, 2.93 mL, 21.05 mmol) was added dropwise at 0° C. under nitrogen. The cold bath removed and the resulting mixture was stirred at room temperature for 16 h. Methanol was removed on a rotary evaporator and the residue was dried under high vacuum. The residue was taken up in water and purified via preparatory HPLC (2-10% acetonitrile in water with 5 mM ammonium acetate). Fractions containing the desired product were combined and lyophilized to dryness to afford Intermediate A-10 as brown solid. Yield: 0.350 g (25%); LC-MS m/z 348.0 [M−H]⁻.

### (2-((2R,3S,4S,5S,6R)-3,4,5-trihydroxy-6-(4-isothiocyanatophenoxy)tetrahydro-2H-pyran-2-yl)ethyl)phosphonic acid (Compound A)

To a stirred solution of Intermediate A-10 (1.0 eq, 1.75 g, 5.01 mmol) in mixture of ethanol:water (7:3) (20 ml) was added thiophosgene (5.00 eq, 1.92 mL, 25.05 mmol) dropwise at 0° C. under nitrogen. The cold bath removed and the resulting mixture was stirred at room temperature under nitrogen for 3 h. Volatiles were removed on a rotary evaporator and the residue was dried under high vacuum. The residue was taken up in water and purified via prep-HPLC (20-40% acetonitrile in water with 5.0 mmol ammonium acetate). Fractions containing the desired product were combined and lyophilized to dryness to afford Compound A as a white solid. Yield: 0.135 g (6.8%) LC-MS m/z 392.08 [M+1]⁺; ¹H NMR (400 MHz, D₂O) δ 7.32 (d, J=8.92 Hz, 2H), 7.12 (d, J=8.96 Hz, 2H), 5.57 (s, 1H), 4.13 (s, 1H), 3.96 (dd, J=9.16, 3.44 Hz, 1H), 3.59-3.48 (m, 2H), 2.03-1.88 (m, 1H), 1.68-1.54 (m, 2H), 1.27-1.15 (m, 1H).

### Example 1: Synthesis of Compound I-1

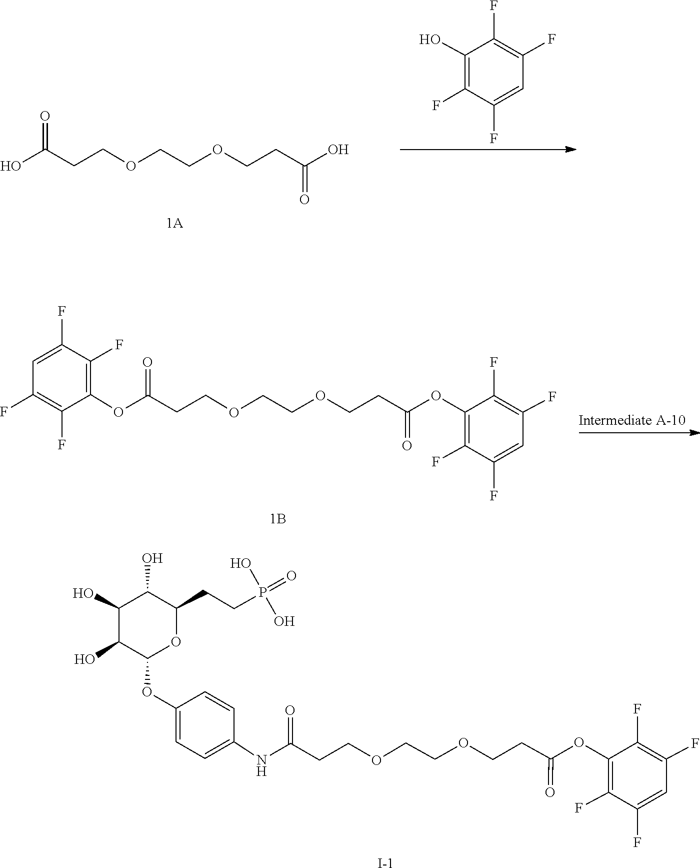 [SMILES: O=C(O)CCOCCOCCC(=O)O ; Oc1c(F)c(F)cc(F)c1F ; O=C(CCOCCOCCC(=O)Oc1c(F)c(F)cc(F)c1F)Oc1c(F)c(F)cc(F)c1F ; O=C(CCOCCOCCC(=O)Oc1c(F)c(F)cc(F)c1F)Nc1ccc(O[C@H]2O[C@H](CCP(=O)(O)O)[C@@H](O)[C@H](O)[C@@H]2O)cc1 ; [I-]]

A solution of 3,3′-(ethane-1,2-diylbis(oxy))dipropionic acid (1A) (1.0 eq, 0.200 g, 0.96 mmol) and 2,3,5,6-tetrafluorophenol (2.0 eq, 0.315 g, 1.9 mmol) in ethyl acetate (4 mL) was cooled at 0° C., N,N′-diisopropylcarbodiimide (3.0 eq, 0.44 mL, 2.8 mmol) was added and reaction mixture was stirred at room temperature for 3 h. Reaction mixture was filtered through Celite bed and Celite bed was washed with ethyl acetate. The filtrate was concentrated to get crude product which was purified by column chromatography using silica gel (100-200 mesh) and 0-10% ethyl acetate in hexane to afford Compound 1B as a colorless viscous liquid. Yield: 0.370 g, 76.1%; LC-MS m/z 500.96 [M−1]⁻.

Intermediate A-10 (1.0 eq, 0.040 g, 0.11 mmol) was dissolved in dimethyl sulfoxide (1 mL) and triethylamine (10.0 eq, 0.15 mL, 1.1 mmol) was added. In another vial, Compound 1B (5.0 eq, 0.276 g, 0.55 mmol) was dissolved in dimethyl sulfoxide (1 mL) and the previous mixture was added dropwise to this mixture (over 30 minutes). Reaction mixture was stirred at room temperature for 5 minutes. After completion, reaction mixture was diluted with acetonitrile and purified by preparatory HPLC (25-45 acetonitrile in water with 0.1% TFA). Fractions containing the desired product were combined and lyophilized to dryness to afford Compound I-1 as an off white solid. Yield: 0.002 g, 2.5%; LC-MS m/z 686.25 [M+1]⁺; ¹H NMR (400 MHz, D₂O) δ 7.35 (d, J=8.88 Hz, 2H), 7.29-7.23 (m, 1H), 7.07 (d, J=8.96 Hz, 2H), 5.50 (s, 1H), 4.13 (bs, 1H), 3.98-3.95 (m, 1H), 3.91 (t, J=5.64 Hz, 2H), 3.86 (t, J=5.72 Hz, 2H), 3.72 (s, 4H), 3.58 (d, J=7.32 Hz, 2H), 2.96 (t, J=5.76 Hz, 2H), 2.66 (t, J=5.8 Hz, 2H), 2.03-2.00 (m, 1H), 1.74-1.63 (m, 2H), 1.32-1.26 (m, 1H).

### Example 2: Synthesis of Compound I-2

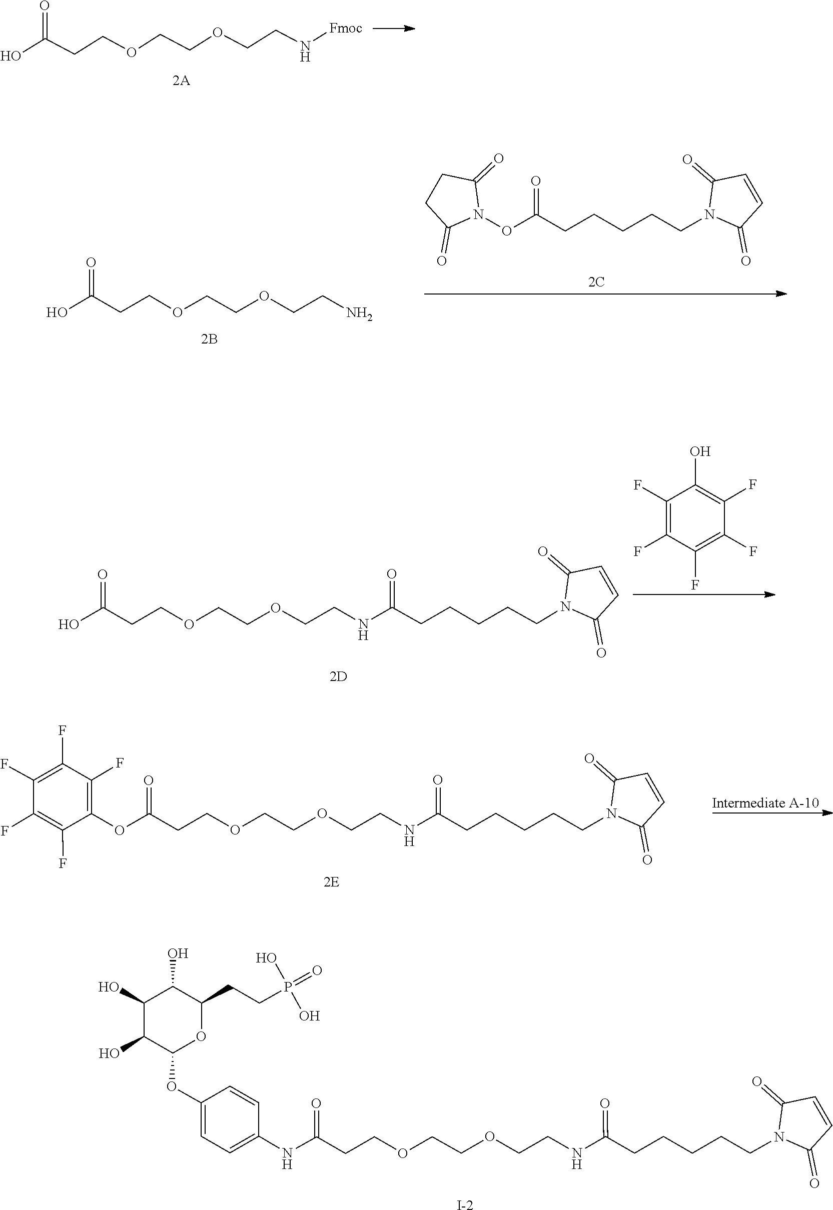

To a stirred solution of 1-(9H-fluoren-9-yl)-3-oxo-2,7,10-trioxa-4-azatridecan-13-oic acid (2A) (2.0 g, 5.00 mmol) in acetonitrile (16 mL), piperidine (4 mL) was added and reaction mixture was stirred for 1 h. The progress of reaction was monitored by TLC. After the completion of reaction, reaction mixture was concentrated to get crude. The crude was washed with hexane and dried to afford Compound 2B as off white semi solid. Yield: 0.85 g, 96%; LC-MS m/z 178.06 [M+1]⁺.

To a stirred solution of 2,5-dioxopyrrolidin-1-yl 6-(2,5-dioxo-2,5-dihydro-1H-pyrrol-1-yl)hexanoate (2C) (1.0 g, 3.24 mmol) and Compound 2B (0.86 g, 4.87 mmol) in N,N-dimethylformamide (20 mL), N,N-diisopropylethylamine (1.46 mL, 8.11 mmol) was added and reaction mixture was stirred for 3 h. The progress of reaction was monitored by LC-MS. After the completion of reaction of reaction mixture was concentrated under reduced pressure to afford crude. The crude was purified by preparatory HPLC (XBS column using 30% ACN in 70% of 5 mM ammonium acetate) to afford Compound 2D as brown oil. Yield: 0.6 g, 43%; LC-MS m/z 371.22 [M+1]⁺.

To a stirred solution of Compound 2D (0.35 g, 0.945 mmol) and pentafluorophenol (0.17 g, 0.945 mmol) in ethyl acetate (10 mL), N,N′-diisopropylcarbodiimide (0.13 g, 1.04 mmol) was added and reaction mixture was stirred for 16 h at room temperature. The progress of reaction was monitored by TLC and LC-MS. After the completion of reaction, reaction mixture filtered through filter cartridge and washed with small amount of ethyl acetate (2 mL) and concentrated under reduced pressure under inert atmosphere to afford crude Compound 2E, which was used without further purification for the next step. Yield: 0.2 g, (crude); LC-MS m/z 537.19 [M+1]⁺.

To a stirred solution of Intermediate A-10 (0.05 g, 0.14 mmol) in dimethyl sulfoxide (2 mL), powdered molecular sieves and Compound 2E (0.11 g, 0.21 mmol), triethylamine (0.04 g, 0.42 mmol) was added dropwise. Reaction mixture was stirred for 16 h at room temperature. The progress of reaction was monitored by LC-MS. The reaction mixture was purified by preparatory HPLC (XB-C-18 column using 40% ACN in 60% of 5 mM ammonium acetate). Fractions containing the desired product were combined and lyophilized to dryness to afford Compound I-2 as white solid. Yield: 0.03 g, 31%; LC-MS m/z 702.31 [M+1]⁺. ¹H NMR (400 MHz, D₂O) δ 7.37 (d, J=8.8 Hz, 2H), 7.11 (d, J=8.9 Hz, 2H), 6.77 (s, 2H), 5.52 (d, J=1.48 Hz, 1H), 5.20 (bs, 1H), 4.14-4.13 (m, 1H), 3.98-3.95 (m, 1H), 3.85 (t, J=5.88 Hz, 2H), 3.72-3.66 (m, 6H), 3.60-3.55 (m, 4H), 3.42 (t, J=6.96 Hz, 2H), 3.29 (t, J=5.28 Hz, 2H), 2.66 (t, J=5.84 Hz, 2H), 2.10 (t, J=7.32 Hz, 2H), 2.04-1.95 (m, 1H), 1.69-1.57 (m, 2H), 1.54-1.45 (m, 4H).

### Example 3: Synthesis of Compound I-3

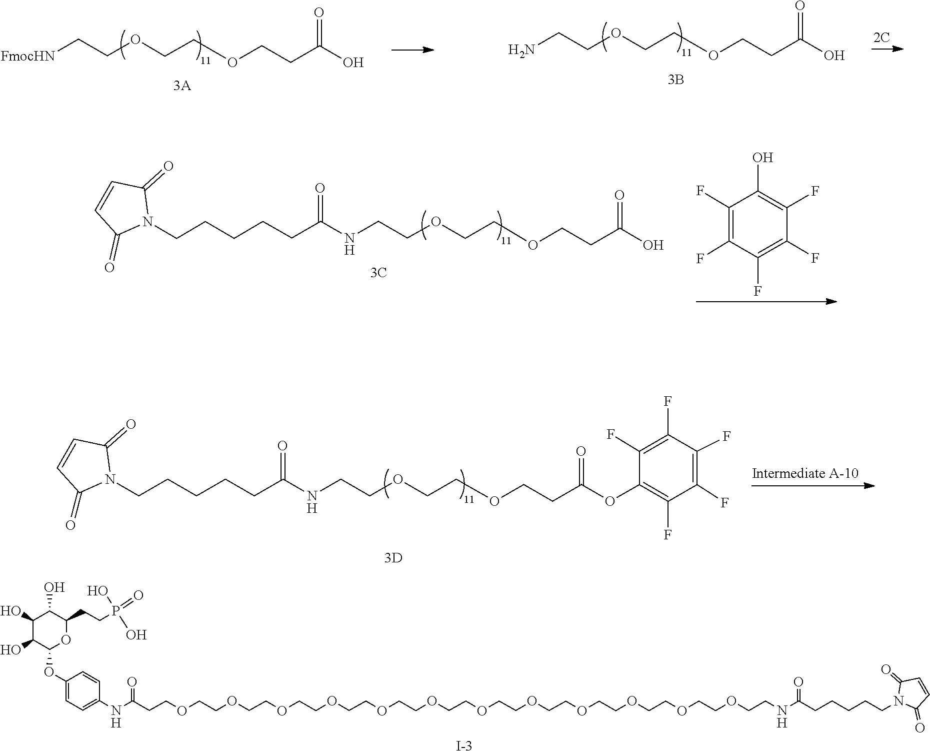

Piperidine (1 mL) was added to a stirred solution of 1-(9H-fluoren-9-yl)-3-oxo-2,7,10,13,16,19,22,25,28,31,34,37,40-tridecaoxa-4-azatritetracontan-43-oic acid (3A) (1.0 g, 1.19 mmol) in acetonitrile (9 mL) at room temperature and reaction were maintained for 1 h. The progress of reaction was monitored by TLC. After the completion, reaction mixture was concentrated to get crude residue. The residue washed with hexane (10 mL×4) and dried under vacuum to afford Compound 3B (0.700 g, 95%) as off white solid. ¹H NMR (400 MHz, Dimethyl Sulfoxide-d6) δ 3.60-3.45 (m, 48H), 2.75 (t, J=5.7 Hz, 2H), 2.28 (t, J=6.7 Hz, 2H).

At room temperature, to the stirred solution of Compound 3B (0.600 g, 0.971 mmol) and 2 C (0.449 g, 1.46 mmol) in N,N-dimethylformamide (10 mL), were added N,N-diisopropylethyl amine (0.448 mL, 2.43 mmol) and reaction was stirred for 3 h. After the completion of reaction, the reaction mixture was concentrated under reduced pressure that afforded thick residue. The residue was purified by preparatory HPLC using XB-C₁₈ (19×250 mm) 10μ column with eluent 20-45% acetonitrile in water with 5 mM ammonium acetate buffer. The desired fractions were combined and freeze dried to afford Compound 3C as pale yellow oil. LC-MS m/z 809.5 [M−1]⁻: Yield: 0.433 g, 55%.

To the stirred solution of Compound 3C (0.15 g, 0.185 mmol) and pentafluorophenol (0.040 g, 0.222 mmol) in ethylacetate (5 mL), N,N′-diisopropylcarbodiimide (0.028 g, 0.222 mmol) was added at 0° C. and reaction mixture was stirred for 16 h at room temperature. The progress of reaction was monitored by TLC and LC-MS. After the completion of reaction, the solid observed due to di-isopropyl urea in the reaction mixture was filtered through filter cartridge and washed with small amount of ethyl acetate (2 mL) and concentrated under vacuo to get crude Compound 3D, which was not further purified for the next step. LC-MS m/z 994.5 [M+H₂O]⁺: Yield: 0.120 g, 66%.

Intermediate A-10 (0.032 g, 0.091 mmol) in dimethyl sulfoxide (0.5 mL) were added drop-wise to a stirred solution of Compound 3D (0.099 g, 0.101 mmol) in dimethyl sulfoxide (0.5 mL) at room temperature and stirred for 5 min. Triethyamine (0.013 g, 0.137 mmol) added to reaction mixture and reaction maintained for 16 h at room temperature followed by prep-HPLC using Sunfire C₁₈ (19×250 mm) 10μ column with eluent 40-60% acetonitrile in water with 0.1% TFA. Fractions containing the desired product were combined and lyophilized to dryness to afford desired Compound I-3 (0.011 g, 10% yield) as a thick syrup. LC-MS m/z 1142.6 [M+1]⁺. ¹H NMR (400 MHz, D2O) δ 7.27 (d, J=9.0 Hz, 2H), 7.16 (d, J=9.0 Hz, 2H) 6.83 (s, 2H), 5.56 (s, 1H), 4.20-4.15 (m, 1H), 4.05-3.98 (m, 1H), 3.88 (t, J=6.0 Hz, 2H), 3.75-3.58 (m, 49H), 3.49 (t, J=6.8 Hz, 2H), 3.37 (t, J=5.6 Hz, 2H), 2.69 (t, J=6.0 Hz, 2H), 2.23 (t, J=7.2 Hz, 2H), 2.10-1.98 (m, 1H), 1.75-1.55 (m, 6H), 5.56 (s, 1H), 1.38-1.20 (m, 2H).

### Example 4: Synthesis of Compound I-4

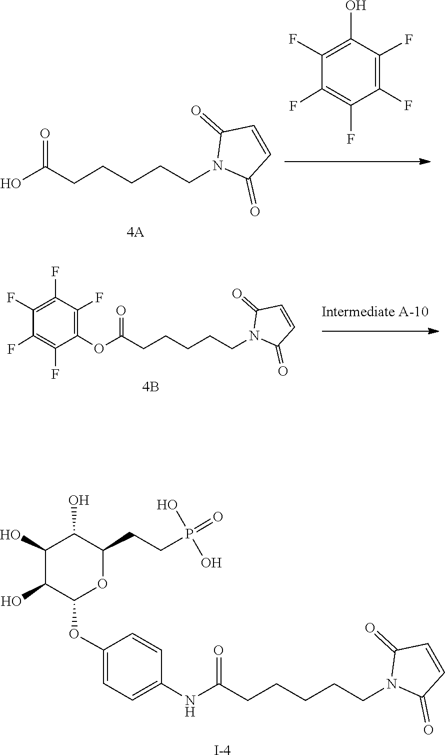

A solution of 6-(2,5-dioxo-2,5-dihydro-1H-pyrrol-1-yl)hexanoic acid (4A) (1.0 eq, 2.5 g, 11.8 mmol) and 2,3,4,5,6-pentafluorophenol (1.0 eq, 2.17 g, 11.8 mmol) in ethyl acetate (50 mL) was cooled at 0° C., N,N′-diisopropylcarbodiimide (1.1 eq, 2.0 mL, 12.9 mmol) was added and reaction mixture was stirred at room temperature for 16 h. Reaction mixture was filtered through Celite bed and washed with ethyl acetate. The filtrate was concentrated to get crude product which was purified by column chromatography using silica gel (100-200 mesh) and 0-25% ethyl acetate in hexane to afford Compound 4B as a white solid. Yield: 3.50 g, 79.5%; LC-MS m/z 377.99 [M+1]⁺.

Intermediate A-10 (1.0 eq, 0.050 g, 0.14 mmol) was dissolved in dimethyl sulfoxide (1 mL), triethylamine (3.0 eq, 0.06 mL, 0.42 mmol) and Compound 4B (2.0 eq, 0.105 g, 0.28 mmol) were added and reaction mixture was stirred at room temperature for 16 h. After completion, reaction mixture was diluted with acetonitrile and purified by prep-HPLC (8-15% acetonitrile in water with 5 mM ammonium acetate). Fractions containing the desired product were combined and lyophilized to dryness to afford Compound I-4 as an off white solid. Yield: 0.006 g, 8.0%; LC-MS m/z 543.27 [M+1]⁺; ¹H NMR (400 MHz, D₂O) δ 7.35 (d, J=8.96 Hz, 2H), 7.16 (d, J=9.0 Hz, 2H), 6.77 (s, 2H), 5.58 (d, J=1.64 Hz, 1H), 4.17-4.16 (m, 1H), 4.02-3.95 (m, 1H), 3.63-3.57 (m, 2H), 3.52 (t, J=6.84 Hz, 2H), 2.39 (t, J=7.24 Hz, 2H), 2.06-1.98 (m, 1H), 1.74-1.58 (m, 6H), 1.37-1.22 (m, 3H).

### Example 5: Synthesis of of Compound I-5

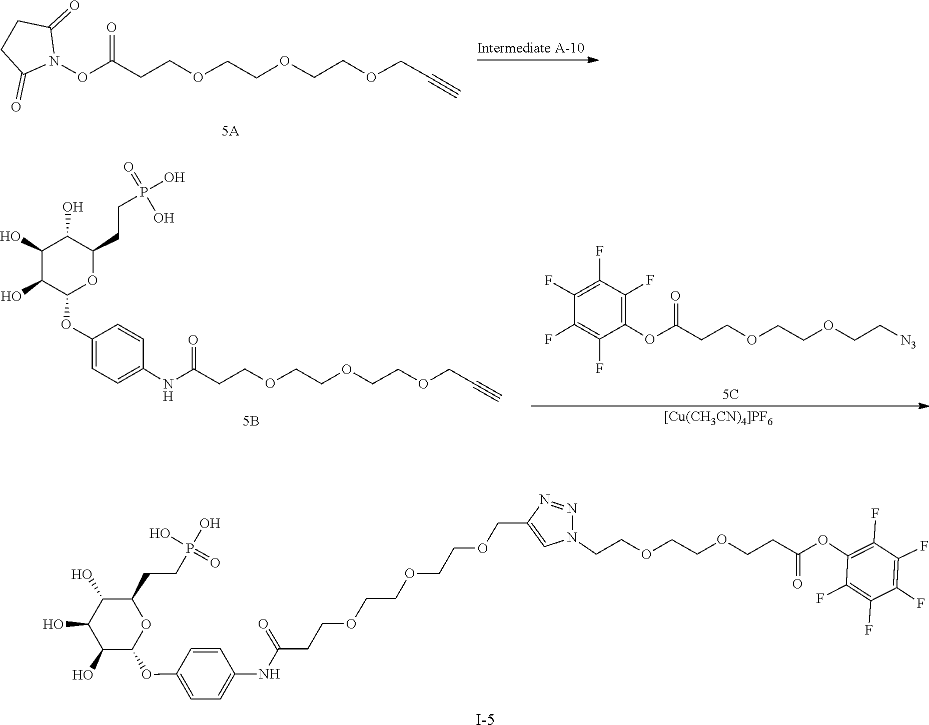

In dimethylsulfoxide (1.0 mL), molecular sieves (Powder, Catalyst support, sodium Y zeolite, Aldrich Cat no. 334448) was added followed by Intermediate A-10 (1.0 eq, 0.060 g, 0.172 mmol), triethylamine (3.0 eq, 0.074 mL, 0.515 mmol) and 2,5-dioxopyrrolidin-1-yl 3-(2-(2-(prop-2-yn-1-yloxy)ethoxy)ethoxy)propanoate (5A) (1.0 eq, 0.053 g, 0.172 mmol) were added and reaction mixture was stirred at room temperature for 3 h. After completion, reaction mixture was diluted with acetonitrile and purified by preparatory HPLC (14-33% acetonitrile in water with 0.1% TFA). Fractions containing the desired product were combined and lyophilized to dryness to afford Compound 5B as an off white sticky solid. Yield: 0.018 g, 17.93%; LC-MS m/z 548.32 [M+1]⁺.

A solution of Compound 5B (1.0 eq, 0.018 g, 0.032 mmol) and perfluorophenyl 3-(2-(2-azidoethoxy)ethoxy)propanoate (5C) (1.2 eq, 0.014 g, 0.039 mmol) in dimethyl sulfoxide (0.6 mL) was stirred at room temperature for 5 minutes. Then, tetrakis(acetonitrile)copper(I) hexafluorophosphate (2.8 eq, 0.034 g, 0.092 mmol) was added and reaction mixture was stirred at room temperature for 1 h. After completion, reaction mixture was diluted with acetonitrile and purified by prep-HPLC (40-60% acetonitrile in water with 0.1% TFA). Fractions containing the desired product were combined and lyophilized to dryness to afford Compound I-5 as a white solid. Yield: 0.015 g, 48.67%; LC-MS m/z 917.37 [M+1]⁺; ¹H NMR (400 MHz, D₂O) δ 7.97 (s, 1H), 7.36 (d, J=9.2 Hz, 2H), 7.08 (d, J=9.2 Hz, 2H), 5.51 (s, 1H), 4.59-4.55 (m, 2H), 4.15-4.14 (m, 1H), 3.97-3.92 (m, 3H), 3.87-3.81 (m, 4H), 3.70-3.57 (m, 14H), 2.97 (t, J=6.0 Hz, 2H), 2.66 (t, J=6.0 Hz, 2H), 2.00 (bs, 1H), 1.71-1.64 (m, 2H), 1.33 (bs, 1H).

### Example 6: Synthesis of of Compound I-6

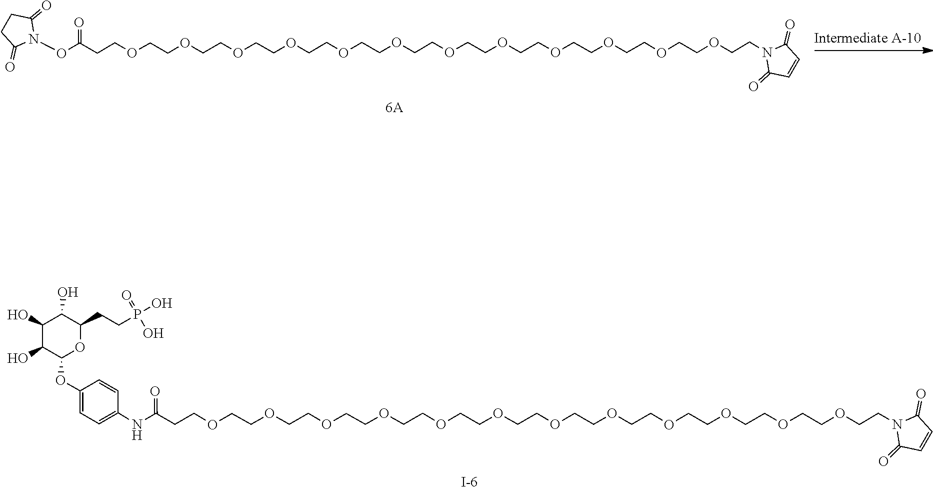

To a stirred solution of Intermediate A-10 (0.02 g, 0.057 mmol) in dimethyl sulfoxide (2 mL), powdered molecular sieves and 2,5-dioxopyrrolidin-1-yl 1-(2,5-dioxo-2,5-dihydro-1H-pyrrol-1-yl)-3,6,9,12,15,18,21,24,27,30,33,36-dodecaoxanonatriacontan-39-oate (6A) 0.07 g, 0.085 mmol), triethylamine (0.018 g, 0.172 mmol) was added dropwise. Reaction mixture was stirred for 16 h at room temperature. The progress of reaction was monitored by LC-MS. The reaction mixture was purified by prep-H PLC (Xselect-Phenylhexyl using 30% ACN and 0.1% TFA in 70% H₂O). Fractions containing the desired product were combined and lyophilized to dryness to afford Compound I-6 as white solid. Yield: 0.0065 g, 11%; LC-MS m/z 1029.58 [M+1]⁺. ¹H NMR (400 MHz, D₂O) δ 7.42 (d, J=8.8 Hz, 2H), 7.16 (d, J=9.2 Hz, 2H), 6.86 (s, 2H), 5.56 (s, 1H), 4.16 (d, J=1.6 Hz 1H), 4.00 (t, J=9.6 Hz 1H), 3.87 (t, J=5.88 Hz, 2H), 3.71-3.61 (m, 50H), 2.69 (t, J=11.6 Hz, 2H), 2.15-1.95 (m, 1H), 1.75-1.61 (m, 2H), 1.43-1.25 (m, 1H).

### Example 7: Synthesis of of Compound I-7

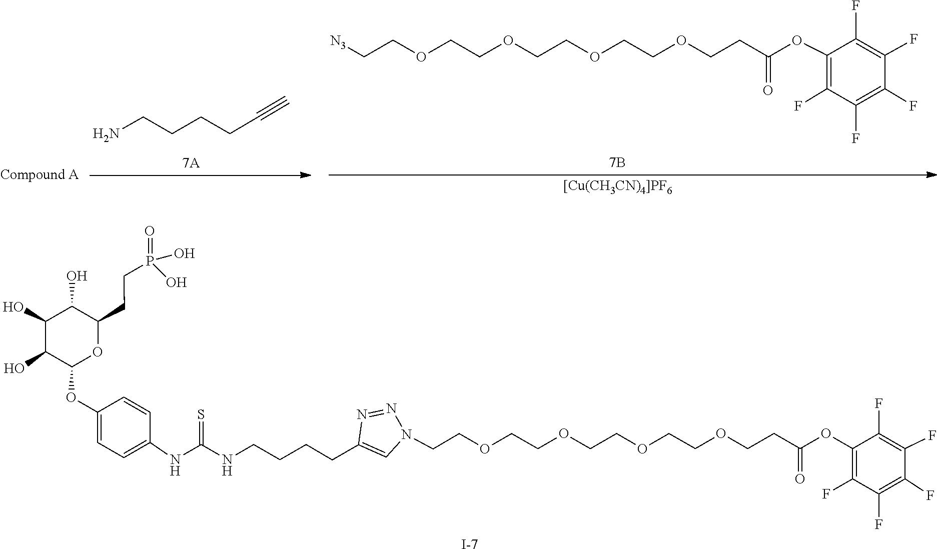

A solution of hex-5-yn-1-amine (7A) (1.20 eq, 3.9 mg, 0.0405 mmol) in NMP (0.15 mL) was added to Compound A (1.00 eq, 13.2 mg, 0.0337 mmol) in a 1 dram vial with a stirbar. The resulting mixture was capped and stirred at room temperature for 30 min (Solids slowly dissolved to give a clear yellow solution). A solution of azido-PEG4-pentafluorophenol ester (7B) (1.50 eq, 23.1 mg, 0.0506 mmol) in NMP (0.20 mL) was added followed by tetrakis(acetonitrile)copper(I) hexafluorophosphate (3.00 eq, 37.7 mg, 0.101 mmol). The resulting clear dark yellow solution was capped and stirred at room temperature for 30 min. The reaction mixture was diluted with mixture of NMP, ethanol, and acetic acid, filtered, and purified via preparatory HPLC (15-60% acetonitrile in water with 0.1% TFA). Fractions containing the desired product were combined and lyophilized to dryness to afford Compound I-7 as a white solid. Yield: 11.1 mg, 35%; LC-MS m/z 946.5 [M+1]⁺; ¹H NMR (300 MHz, DMSO-d₆ with D₂O) δ 7.80 (s, 1H), 7.25 (d, J=8.4 Hz, 2H), 6.98 (d, J=8.4 Hz, 2H), 5.32 (s, 1H), 4.44 (s, 2H), 3.86-3.68 (m, 5H), 3.67-3.23 (m, 17H), 3.05-2.91 (m, 2H), 2.67-2.56 (m, 2H), 2.00-1.81 (m, 1H), 1.69-1.41 (m, 6H), 1.30-1.07 (m, 1H).

### Example 8: Synthesis of of Compound I-8

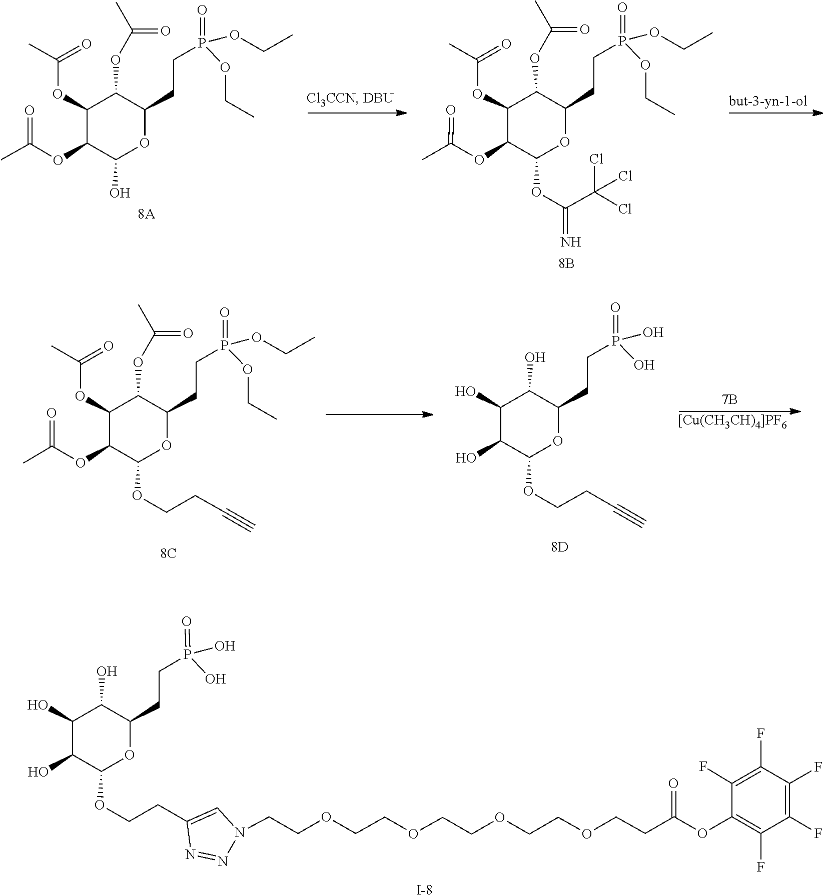

DBU (0.05 eq, 0.025 mL, 0.168 mmol) was added to a stirred solution of (2R,3R,4S,5S,6S)-2-(2-(diethoxyphosphoryl)ethyl)-6-hydroxytetrahydro-2H-pyran-3,4,5-triyl triacetate (8A) (1.00 eq, 1.48 g, 3.36 mmol) and trichloroacetonitrile (10.0 eq, 3.4 mL, 33.6 mmol) in DCM (30 mL) at 0° C. under nitrogen. The resulting mixture was stirred at 0° C. under nitrogen. More DBU (0.0500 eq, 0.025 mL, 0.168 mmol) was added and the cold bath was removed. The resulting mixture was stirred at room temperature for 45 min. Most of the solvent was removed on a rotary evaporator. The residue was loaded onto a silica gel loading column which was pre-equilibrated with 0.1% triethylamine in dichloromethane and purified via silica gel chromatography (column pre-equilibrated with 0.1% triethylamine in 30% ethyl acetate/hexanes) (30-100% ethyl acetate in hexanes). Fractions containing the desired product were combined and concentrated on a rotary evaporator. The residue was stripped down from dry dichloromethane twice, dried under high vacuum for 30 min, and then stored under nitrogen at −80° C. to afford Compound 8B as a colorless semi-solid. Yield: 1.26 g, 64%; ¹H NMR (300 MHz, Chloroform-d) δ 8.74 (s, 1H), 6.21 (s, 1H), 5.45 (s, 1H), 5.34 (t, J=11.2 Hz, 1H), 5.20 (t, J=10.0 Hz, 1H), 4.16-4.00 (m, 4H), 4.00-3.88 (m, 1H), 2.18 (s, 3H), 2.07 (s, 3H), 2.00 (s, 3H), 1.95-1.64 (m, 4H), 1.31 (t, J=7.3 Hz, 6H).

Compound 8B (1.00 eq, 1.25 g, 2.14 mmol) was dissolved in dry DCM (10 mL) with stirring under nitrogen. But-3-yn-1-ol (2.00 eq, 0.32 mL, 4.28 mmol) was added and the resulting mixture was cooled to −78° C. with stirring under nitrogen. A solution of boron trifluoride diethyl etherate (0.500 eq, 0.13 mL, 1.07 mmol) in dichloromethane (5 mL) was added slowly. The −78° C. cold bath was removed and the reaction mixture was allowed to slowly warm under nitrogen for 50 min. The reaction mixture was cooled with a water/ice bath and allowed to stir an additional 30 min at 0° C. under nitrogen and then worked up. The reaction mixture was partitioned between dichloromethane and saturated aqueous sodium bicarbonate. The water layer was extracted again with dichloromethane. The combined organics were dried over sodium sulfate, filtered, and purified via silica gel chromatography (20-100% ethyl acetate in dichloromethane) to afford Compound 8C as a colorless viscous oil. Yield: 408 mg, 39%; LC-MS m/z 493.4 [M+1]⁺; ¹H NMR (300 MHz, Chloroform-d) δ 5.35-5.19 (m, 2H), 5.09 (t, J=9.9 Hz, 1H), 4.79 (s, 1H), 4.21-3.98 (m, 4H), 3.91-3.68 (m, 2H), 3.64-3.50 (m, 1H), 2.55-2.44 (m, 2H), 2.15 (s, 3H), 2.05 (s, 3H), 1.98 (s, 3H), 2.07-1.62 (m, 5H), 1.32 (t, J=7.2 Hz, 6H).

Bromotrimethylsilane (5.00 eq, 0.47 mL, 3.57 mmol) was added slowly to a stirred solution of Compound 8C (1.00 eq, 352 mg, 0.715 mmol) in MeCN (7 mL) at 0° C. under nitrogen. The cold bath was removed and the resulting mixture was stirred at room temperature under nitrogen for 3.5 h. Volatiles were removed on a rotary evaporator and the residue was dried under high vacuum briefly. The residue was dissolved in methanol (7 mL) with stirring under nitrogen and sodium methoxide (25 wt % in methanol) (2.50 eq, 0.41 mL, 1.79 mmol) was added. The resulting mixture was stirred at room temperature under nitrogen for 1 h. Acetic acid (3.00 eq, 0.12 mL, 2.14 mmol) was added and then volatiles were removed on a rotary evaporator. The residue was taken up in water and purified via preparatory HPLC (0-15% acetonitrile in water with 0.1% TFA). Most of the solvent was removed on a rotary evaporator at 30° C. and then the remainder was lyophilized to dryness to afford Compound 8D as a white solid. Yield: 208 mg, 94%; LC-MS m/z 311.3 [M+1]⁺; ¹H NMR (300 MHz, Deuterium Oxide) δ 4.88-4.80 (m, 1H), 3.93 (s, 1H), 3.84-3.70 (m, 2H), 3.70-3.56 (m, 2H), 3.48 (t, J=9.7 Hz, 1H), 2.57-2.44 (m, 2H), 2.37 (s, 1H), 2.15-1.61 (m, 4H).

Compound 8D (1.00 eq, 10.0 mg, 0.032 mmol) and azido-PEG4-pentafluorophenol ester 7B (1.20 eq, 17.7 mg, 0.039 mmol) were dissolved in NMP (0.3 mL) with stirring. After 2 min tetrakis(acetonitrile)copper(I) hexafluorophosphate (2.80 eq, 33.6 mg, 0.090 mmol) was added. The resulting light yellow solution was capped and stirred at room temperature for 30 min (slowly turned more green-colored). The reaction mixture was diluted with mixture of NMP, ethanol, and acetic acid, filtered, and purified via preparatory HPLC (15-65% acetonitrile in water with 0.1% TFA). Fractions containing the desired product were combined and lyophilized to dryness to afford Compound I-8 as a white solid. Yield: 12.3 mg, 50%; LC-MS m/z 768.5 [M+1]⁺; ¹H NMR (300 MHz, DMSO-d₆) δ 7.81 (s, 1H), 4.59 (s, 1H), 4.44 (bs, 2H), 3.60-3.30 (m, 17H), 3.27-2.76 (m, 9H), 2.01-1.84 (m, 1H), 1.77-1.58 (m, 1H), 1.56-1.32 (m, 2H).

### Example 9: Synthesis of of Compound I-9

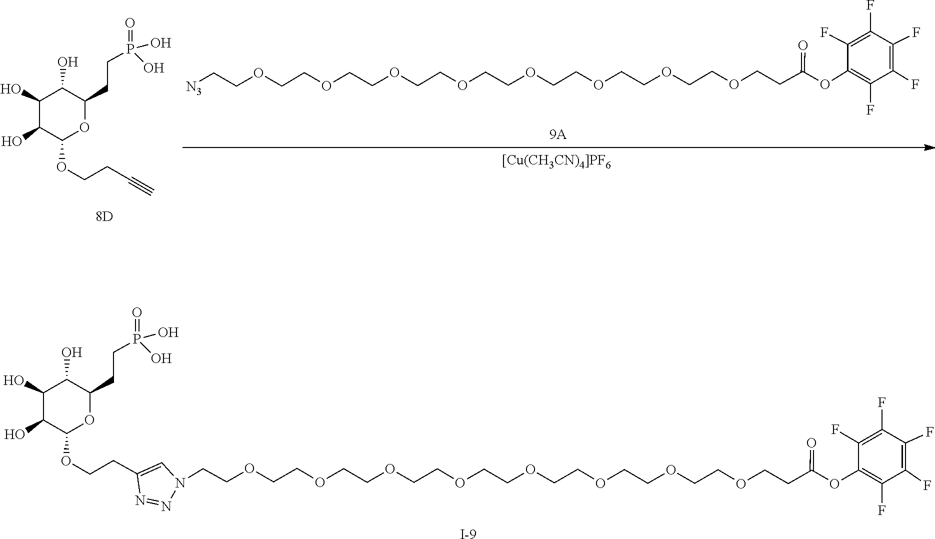

Compound 8D (1.00 eq, 9.8 mg, 0.0316 mmol) and azido-PEG8-pentafluorophenol ester (9A) (1.20 eq, 24.0 mg, 0.0379 mmol) were dissolved in NMP (0.3000 mL) with stirring. After 2 min tetrakis(acetonitrile)copper(I) hexafluorophosphate (2.80 eq, 33.0 mg, 0.0884 mmol) was added. The resulting light yellow solution was capped and stirred at room temperature for 30 min (slowly turned more green-colored). The reaction mixture was diluted with mixture of NMP, ethanol, and acetic acid, filtered, and purified via preparatory HPLC (15-65% acetonitrile in water with 0.1% TFA). Fractions containing the desired product were combined and lyophilized to dryness to afford Compound I-9 as a white solid. Yield: 18.9 mg, 63%; LC-MS m/z 944.6 [M+1]⁺; ¹H NMR (300 MHz, DMSO-d₆ with D₂O) δ 7.81 (s, 1H), 4.59 (s, 1H), 4.44 (s, 2H), 3.86-3.29 (m, 34H), 3.29-2.69 (m, 8H), 2.01-1.80 (m, 1H), 1.80-1.57 (m, 1H), 1.56-1.30 (m, 2H).

### Example 10: Synthesis of Compound I-10

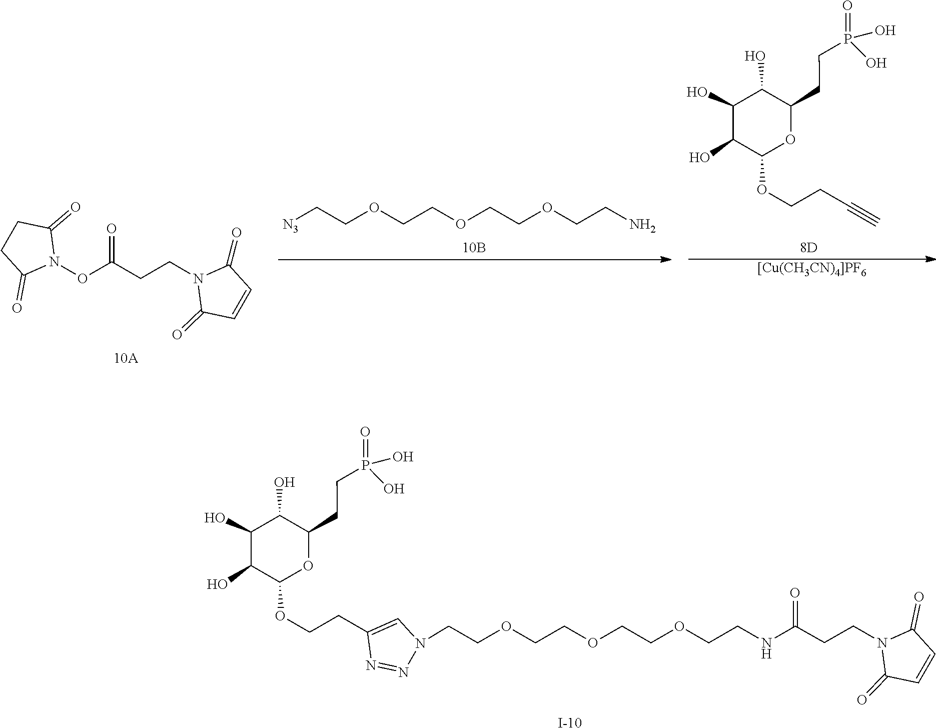

A solution of azido-PEG3-amine (10B) (1.30 eq, 14.3 mg, 0.0654 mmol) in NMP (0.3000 mL) was added to 2,5-dioxopyrrolidin-1-yl 3-(2,5-dioxo-2,5-dihydro-1H-pyrrol-1-yl)propanoate (10A) (1.30 eq, 17.4 mg, 0.0654 mmol) in a 1 dram vial with a stirbar. The resulting clear colorless solution was capped and stirred at room temperature for 30 min and then added to Compound 8D (1.00 eq, 15.6 mg, 0.0503 mmol) in a 1 dram vial with a stirbar. After 2 min, tetrakis(acetonitrile)copper(I) hexafluorophosphate (2.80 eq, 52.5 mg, 0.141 mmol) was added. The resulting light yellow solution was capped and stirred at room temperature for 30 min. The reaction mixture was diluted with mixture of NMP, ethanol, and acetic acid, filtered, and purified via preparatory HPLC (5-40% acetonitrile in water with 0.1% TFA). Fractions containing the desired product were combined and lyophilized to dryness to afford Compound I-10 as a white solid. Yield: 17.7 mg, 52%; LC-MS m/z 680.5 [M+1]⁺; ¹H NMR (300 MHz, DMSO-d₆ with D₂O) δ 7.81 (s, 1H), 6.92 (s, 2H), 4.59 (s, 1H), 4.44 (s, 2H), 3.63-3.26 (m, 15H), 3.26-2.70 (m, 9H), 2.36-2.21 (m, 2H), 2.05-1.83 (m, 1H), 1.79-1.60 (m, 1H), 1.54-1.30 (m, 2H).

### Example 11: Synthesis of Compound I-11

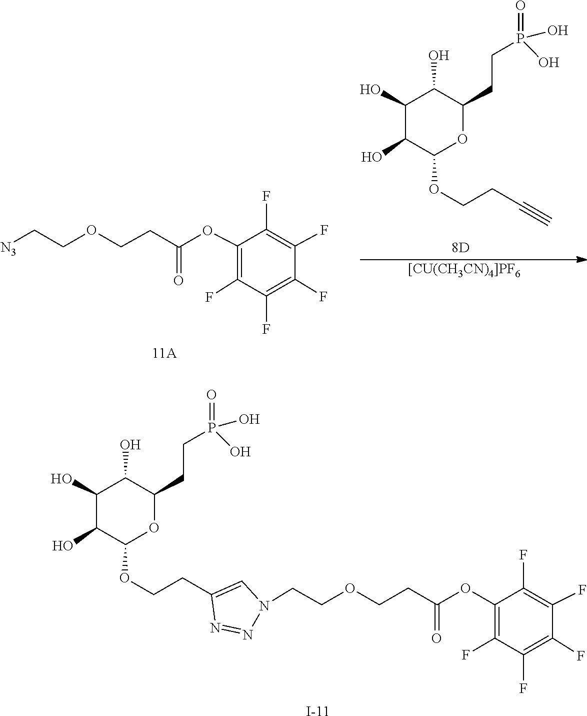

Compound 8D (1.00 eq, 13.4 mg, 0.0432 mmol) and azido-PEG1-pentafluorophenol ester (11A) (1.20 eq, 16.9 mg, 0.0518 mmol) were dissolved in NMP (0.3000 mL) with stirring. After 2 min tetrakis(acetonitrile)copper(I) hexafluorophosphate (2.80 eq, 45.1 mg, 0.121 mmol) was added. The resulting light yellow solution was capped and stirred at room temperature for 30 min. The reaction mixture was diluted with mixture of NMP, ethanol, and acetic acid, filtered, and purified via preparatory HPLC (10-50% acetonitrile in water with 0.1% TFA). Fractions containing the desired product were combined and lyophilized to dryness to afford Compound I-11 as a white solid. Yield: 14.9 mg, 54%; LC-MS m/z 636.4 [M+1]⁺; ¹H NMR (300 MHz, DMSO-d₆ with D₂O) δ 7.75 (s, 1H), 4.57 (s, 1H), 4.51-4.35 (m, 2H), 3.84-3.65 (m, 5H), 3.60-3.45 (m, 2H), 3.41-3.29 (m, 1H), 3.21 (t, J=9.3 Hz, 1H), 3.15-3.03 (m, 1H), 3.03-2.88 (m, 2H), 2.88-2.74 (m, 2H), 2.02-1.82 (m, 1H), 1.79-1.59 (m, 1H), 1.56-1.28 (m, 2H).

### Example 12: Synthesis of Compound I-4

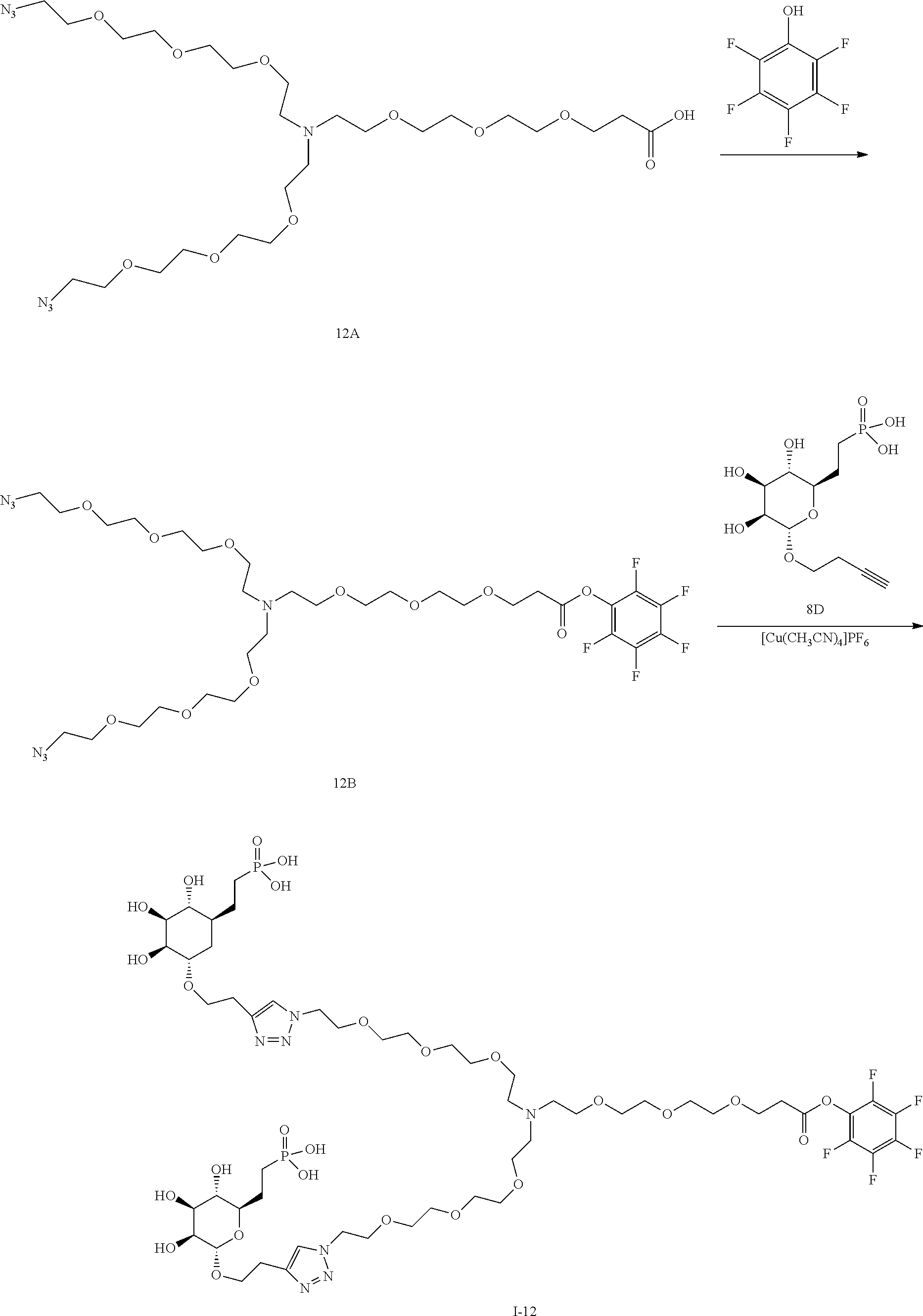

N-(acid-PEG3)-N-bis(PEG3-azide) (12A) (1.00 eq, 18.3 mg, 0.0293 mmol) and N,N′-dicyclohexylcarbodiimide (DCC) (1.00 eq, 6.1 mg, 0.0293 mmol) were dissolved with stirring in NMP (0.1 mL). After 5 min a solution of 2,3,4,5,6-pentafluorophenol (1.50 eq, 8.1 mg, 0.0440 mmol) in NMP (0.2 mL) was added. The resulting clear solution was capped and stirred at room temperature for 2 h at which time a catalytic amount of DMAP was added (white precipitate slowly forms). More DCC (3 mg+1 mg) was added after 16 h and 23 h. 24 h later the resulting mixture was added to Compound 8D (2.00 eq, 18.2 mg, 0.0587 mmol) in a 1 dram vial with a stirbar. After 2 min, tetrakis(acetonitrile)copper(I) hexafluorophosphate (5.00 eq, 54.7 mg, 0.147 mmol) was added. The resulting light yellow solution was capped and stirred at room temperature for 30 min. The reaction mixture was diluted with a mixture of NMP, ethanol, and acetic acid, filtered, and purified via preparatory HPLC (10-40% acetonitrile in water with 0.1% TFA). Fractions containing the desired product were combined and lyophilized to dryness to afford Compound I-12 as a white solid. Yield: 8.7 mg, 21%; LC-MS m/z 1410.9 [M+1]⁺; ¹H NMR (300 MHz, DMSO-d₆ with D₂O) δ 7.81 (s, 2H), 4.60 (s, 2H), 4.45 (s, 4H), 3.87-2.76 (m, 50H), 2.03-1.83 (m, 2H), 1.79-1.59 (m, 2H), 1.55-1.29 (m, 4H).

### Example 13: Synthesis of Compound I-13

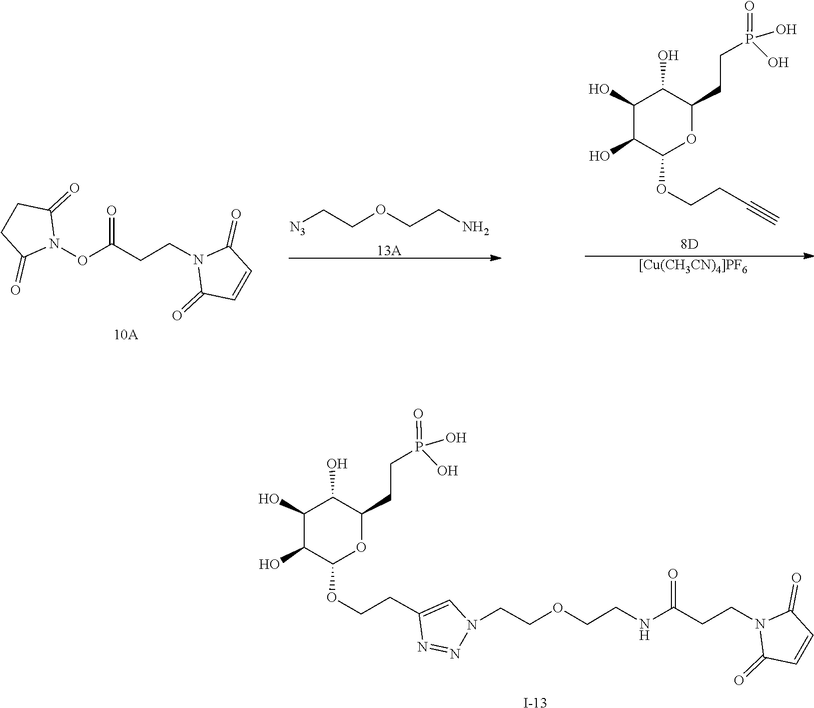

A solution of azido-PEG1-amine (13A) (1.30 eq, 8.5 mg, 0.0649 mmol) in NMP (0.3000 mL) was added to Compound 10A (1.30 eq, 17.3 mg, 0.0649 mmol) in a 1 dram vial with a stirbar. The resulting clear colorless solution was capped and stirred at room temperature for 30 min and then added Compound 8D (1.00 eq, 15.5 mg, 0.0500 mmol) in a 1 dram vial with a stirbar. After 2 min tetrakis(acetonitrile)copper(I) hexafluorophosphate (2.80 eq, 52.1 mg, 0.140 mmol) was added. The resulting light yellow solution was capped and stirred at room temperature for 30 min. The reaction mixture was diluted with mixture of NMP, ethanol, and acetic acid, filtered, and purified via preparatory HPLC (5-30% acetonitrile in water with 0.1% TFA). Fractions containing the desired product were combined and lyophilized to dryness to afford Compound I-13 as a white solid. Yield: 15.0 mg, 51%; LC-MS m/z 592.4 [M+1]⁺; ¹H NMR (300 MHz, DMSO-d₆ with D₂O) δ 7.81 (s, 1H), 6.95 (s, 2H), 4.60 (s, 1H), 4.52-4.36 (m, 2H), 3.80-3.51 (m, 6H), 3.42-3.29 (m, 3H), 3.27-3.03 (m, 5H), 2.91-2.78 (m, 2H), 2.37-2.23 (m, 2H), 2.01-1.85 (m, 1H), 1.79-1.60 (m, 1H), 1.54-1.33 (m, 2H).

### Example 14: Synthesis of Compound I-14

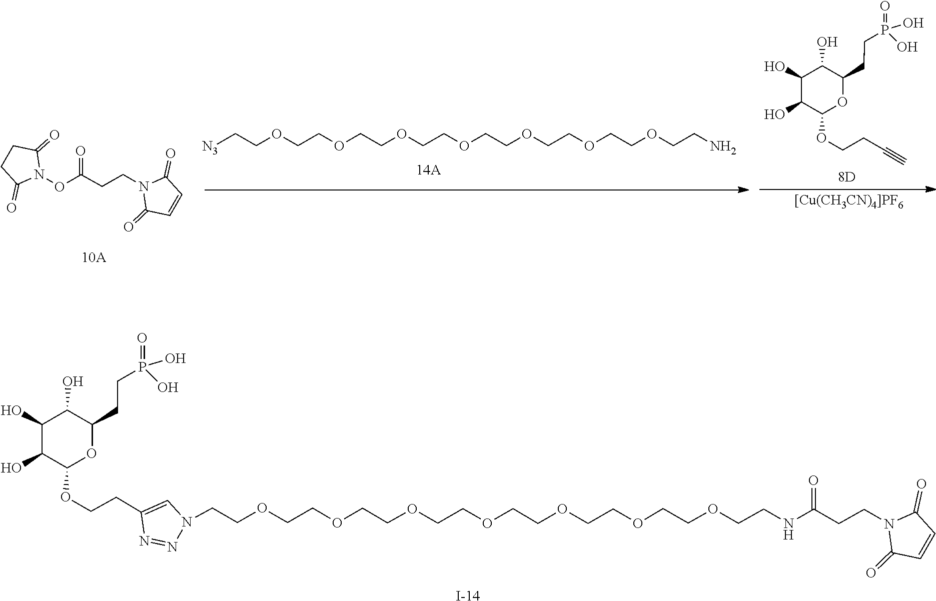

A solution of azido-PEG7-amine (14A) (1.00 eq, 16.9 mg, 0.0429 mmol) in NMP (0.3000 mL) was added to Compound 10A (1.00 eq, 11.4 mg, 0.0429 mmol) in a 1 dram vial with a stirbar. The resulting clear colorless solution was capped and stirred at room temperature for 30 min and then added to Compound 8D (1.00 eq, 13.3 mg, 0.0429 mmol) in a 1 dram vial with a stirbar. After 2 min, tetrakis(acetonitrile)copper(I) hexafluorophosphate (2.80 eq, 44.7 mg, 0.120 mmol) was added. The resulting light yellow solution was capped and stirred at room temperature for 30 min. The reaction mixture was diluted with mixture of NMP, ethanol, and acetic acid, filtered, and purified via preparatory HPLC (5-35% acetonitrile in water with 0.1% TFA). Fractions containing the desired product were combined and lyophilized to dryness to afford Compound I-14 as a white solid. Yield: 8.6 mg, 23%; LC-MS m/z 856.5 [M+1]⁺; ¹H NMR (300 MHz, DMSO-d₆ with D₂O) δ 8.04 (bs, 1H), 7.83 (s, 1H), 6.97 (s, 2H), 4.60 (s, 1H), 4.52-4.38 (m, 2H), 3.84-3.66 (m, 4H), 3.52-3.28 (m, 29H), 3.28-3.04 (m, 5H), 2.85 (t, J=6.7 Hz, 2H), 2.31 (t, J=7.4 Hz, 2H), 2.03-1.86 (m, 1H), 1.80-1.60 (m, 1H), 1.56-1.29 (m, 2H).

### Example 15: Synthesis of Compound I-15

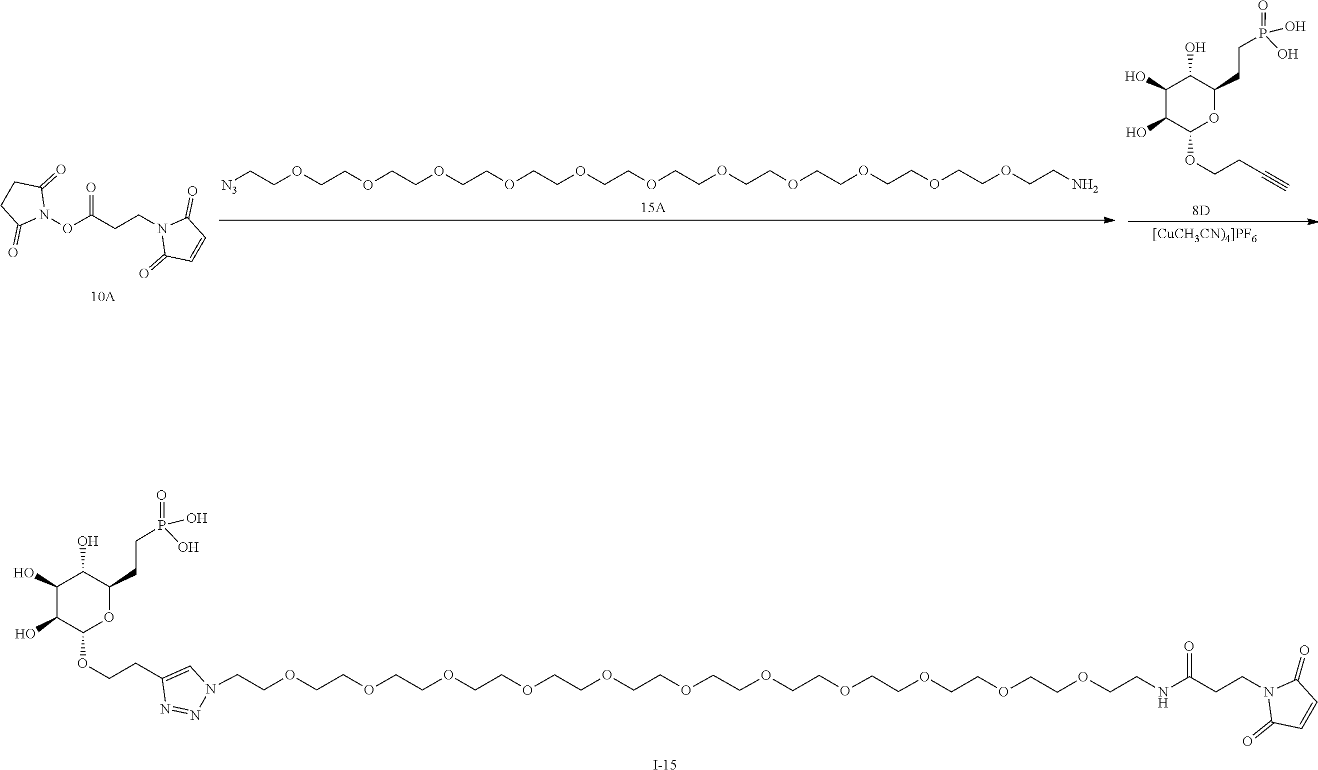

A solution of Azido-PEG11-amine (15A) (1.00 eq, 24.8 mg, 0.0435 mmol) in NMP (0.3000 mL) was added to Compound 10A (1.00 eq, 11.6 mg, 0.0435 mmol) in a 1 dram vial with a stirbar. The resulting clear colorless solution was capped and stirred at room temperature for 30 min and then added to Compound 8D (1.00 eq, 13.5 mg, 0.0435 mmol) in a 1 dram vial with a stirbar. After 2 min, tetrakis(acetonitrile)copper(I) hexafluorophosphate (2.80 eq, 45.4 mg, 0.122 mmol) was added. The resulting light yellow solution was capped and stirred at room temperature for 30 min. The reaction mixture was diluted with mixture of NMP, ethanol, and acetic acid, filtered, and purified via preparatory HPLC (5-35% acetonitrile in water with 0.1% TFA). Fractions containing the desired product were combined and lyophilized to dryness to afford Compound I-15 as a colorless semi-solid. Yield: 17.2 mg, 38%; LC-MS m/z 1032.6 [M+1]⁺; ¹H NMR (300 MHz, DMSO-d₆ with D₂O) δ 7.81 (s, 1H), 6.91 (s, 2H), 4.59 (s, 1H), 4.50-4.36 (m, 2H), 3.91-3.65 (m, 19H), 3.62-3.27 (m, 30H), 3.27-3.03 (m, 5H), 2.91-2.78 (m, 2H), 2.30 (t, J=7.4 Hz, 2H), 1.99-1.85 (m, 1H), 1.80-1.60 (m, 1H), 1.55-1.33 (m, 2H).

### Example 16: Synthesis of Compound I-16

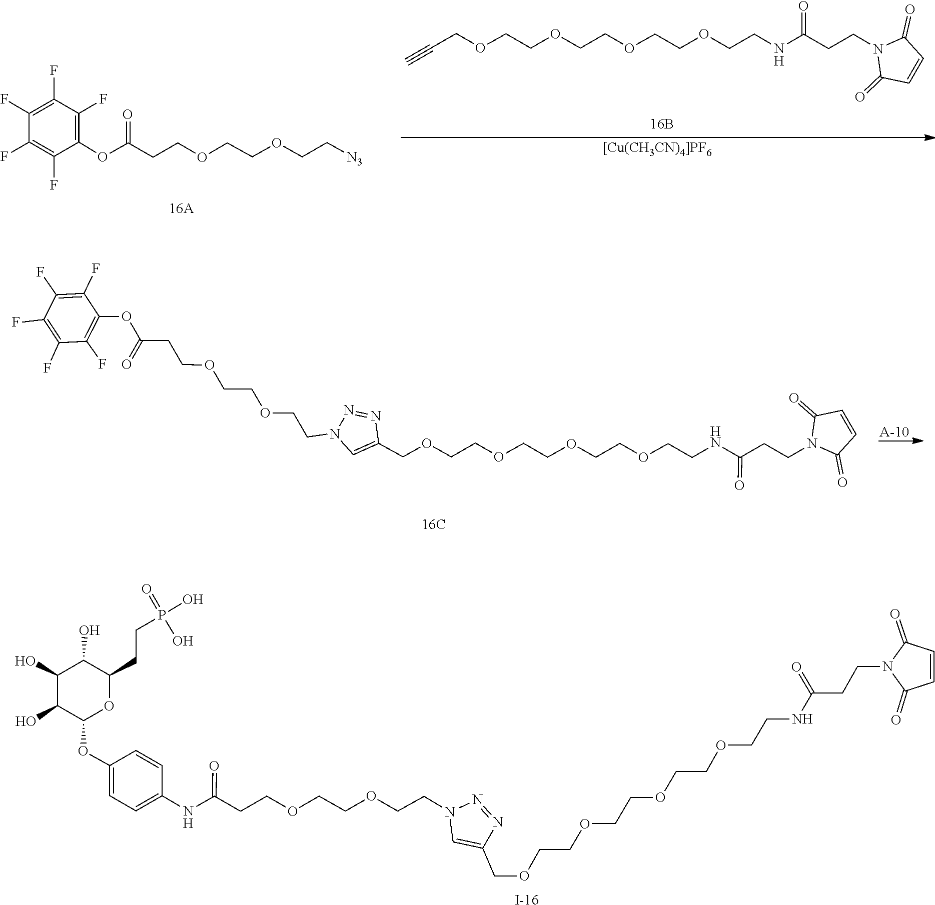

To a solution of perfluorophenyl 3-(2-(2-azidoethoxy)ethoxy)propanoate (16A) (1.0 eq, 0.200 g, 0.542 mmol) in dimethylsulfoxide (4 mL), 3-(2,5-dioxo-2,5-dihydro-1H-pyrrol-1-yl)-N-(3,6,9,12-tetraoxapentadec-14-yn-1-yl)propanamide (16B) (1.5 eq, 0.311 g, 0.812 mmol) was added and stirred for 5 minutes, then tetrakis(acetonitrile)copper(I) hexafluorophosphate (2.8 eq, 0.565 g, 1.52 mmol) was added and reaction mixture was stirred at room temperature for 1 h. After completion, reaction mixture was diluted with acetonitrile and purified by prep HPLC (45-75 acetonitrile in water with 0.1% TFA). Fractions containing the desired product were combined and lyophilized to dryness to afford Compound 16C as a colourless viscous liquid. Yield: 0.045 g, 10.88%; LC-MS m/z 752.33 [M+1]⁺.

In dimethylsulfoxide (0.6 mL), molecular sieves (Powder, Catalyst support, sodium Y zeolite, Aldrich Cat no 334448) was added followed by Intermediate A-10 (1.0 eq, 0.019 g, 0.054 mmol), triethylamine (3.0 eq, 0.023 mL, 0.163 mmol) and Compound 16C (1.1 eq, 0.045 g, 0.059 mmol) were added and reaction mixture was stirred at room temperature for 3 h. After completion, reaction mixture was diluted with acetonitrile and purified by prep HPLC (13-23% acetonitrile in water with 0.1% TFA). Fractions containing the desired product were combined and lyophilized to dryness to afford Compound I-16 as an off white solid. Yield: 0.008 g, 15.82%; LC-MS m/z 917.33 [M+1]⁺; ¹H NMR (400 MHz, D₂O) δ 7.98 (s, 1H), 7.37 (d, J=8.8 Hz, 2H), 7.13 (d, J=8.8 Hz, 2H), 6.83 (s, 2H), 5.55 (s, 1H), 4.61 (s, 2H), 4.56-4.54 (m, 2H), 4.17-4.16 (m, 1H), 4.00-3.98 (m, 1H), 3.94 (t, J=4.8 Hz, 2H), 3.82-3.75 (m, 4H), 3.68-3.58 (m, 18H), 3.53 (t, J=5.2 Hz, 2H), 3.29 (t, J=5.6 Hz, 2H), 2.64 (t, J=6.0 Hz, 2H), 2.48 (t, J=6.4 Hz, 2H), 2.15-1.90 (m, 1H), 1.80-1.60 (m, 2H), 1.40-1.25 (m, 1H).

### Example 17: Synthesis of Compound I-17

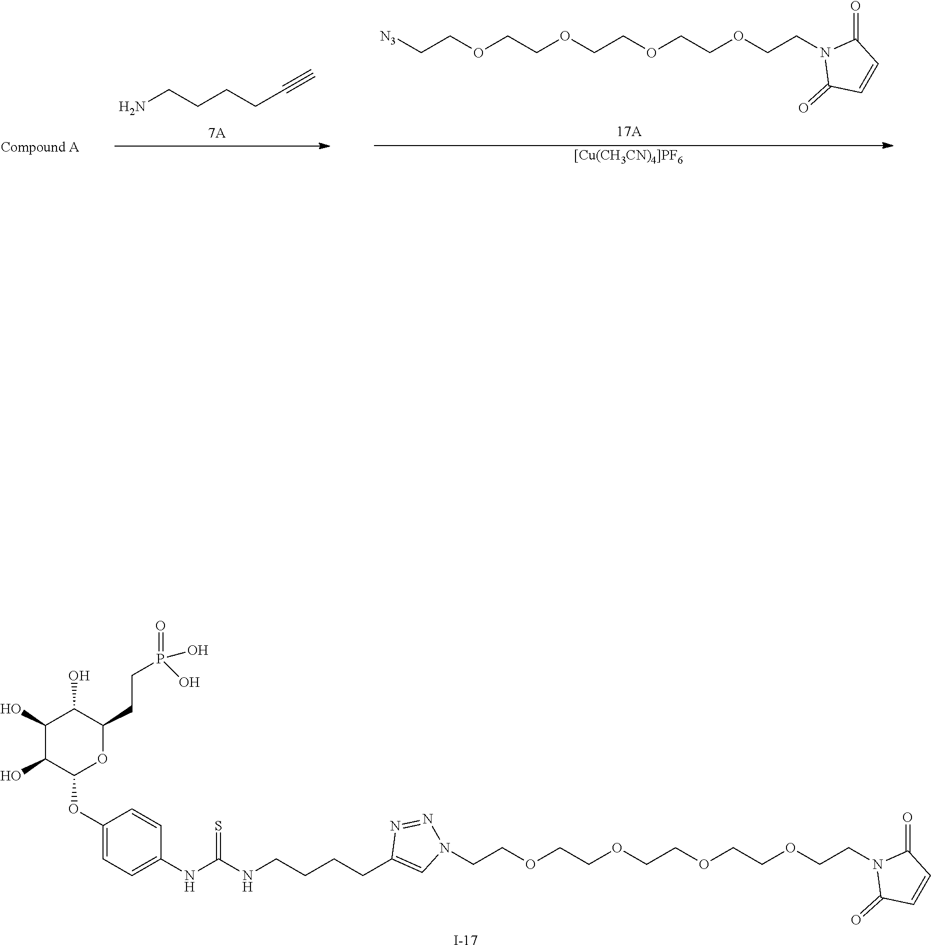

Compound I-17 is synthesized employing the procedures described for Compound I-7 using 1-(14-azido-3,6,9,12-tetraoxatetradecyl)-1H-pyrrole-2,5-dione (17A) in lieu of Compound 7B.

### Example 18: Synthesis of Compound I-18

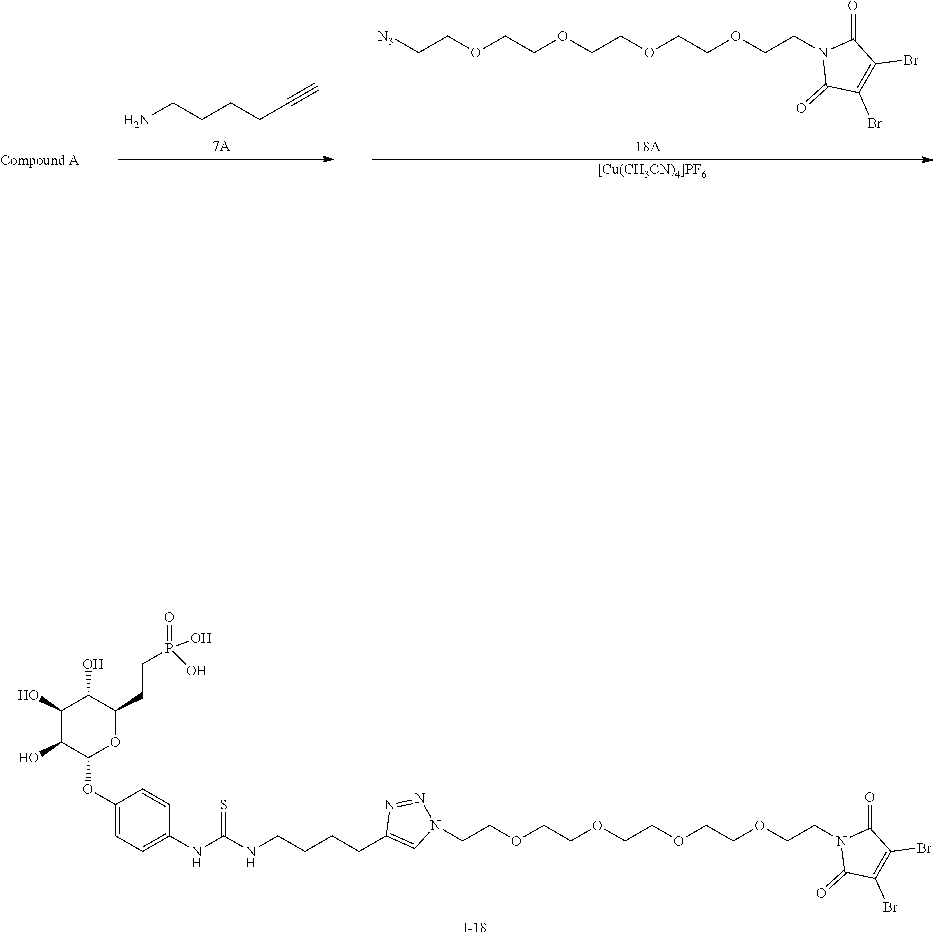

Compound I-18 is synthesized employing the procedures described for Compound I-7 using 1-(14-azido-3,6,9,12-tetraoxatetradecyl)-3,4-dibromo-1H-pyrrole-2,5-dione (18A) in lieu of Compound 7B.

### Example 19: Synthesis of Intermediates X-A, X-B, and X-C

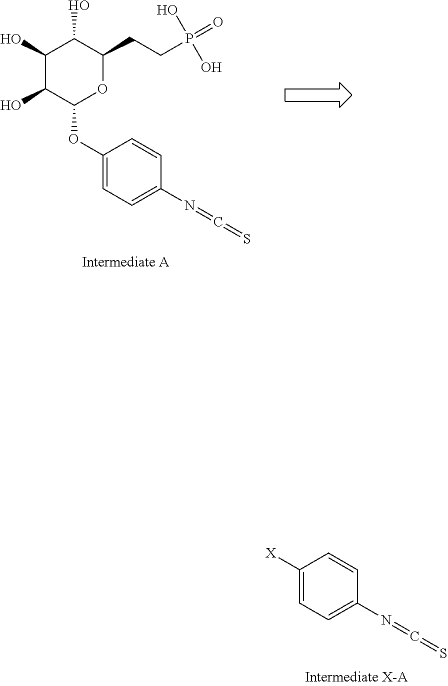

Intermediate X-A are synthesized employing the procedures described for Intermediate A using Compound X-H as the starting material in lieu of mannose 6-phosphate.

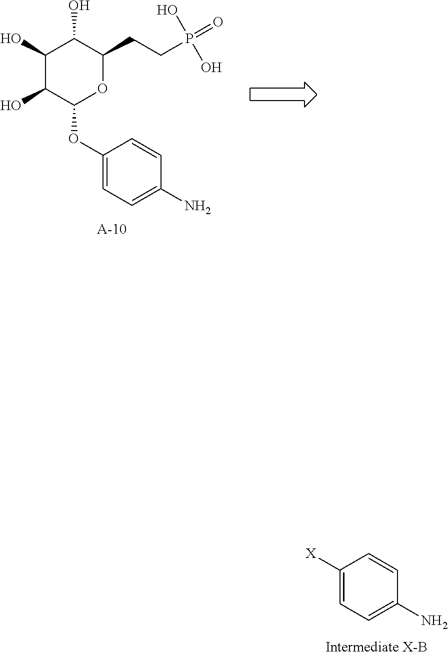

Intermediate X-B are synthesized employing the procedures described for Intermediate A-10 using X-H as the starting material in lieu of mannose 6-phosphate.

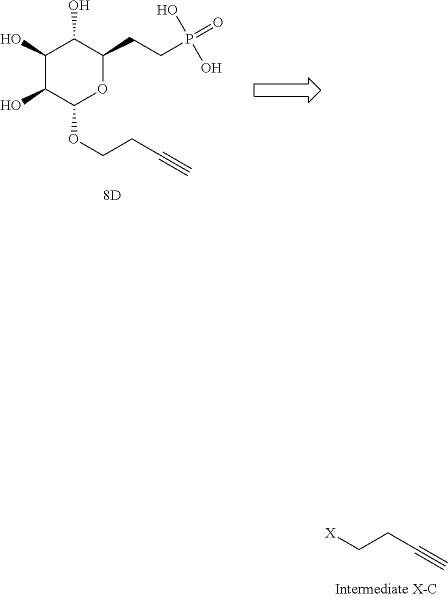

Intermediate X-C are synthesized employing the procedures described for Compound 8D using X-H as the starting material in lieu of mannose 6-phosphate.

### Example 20

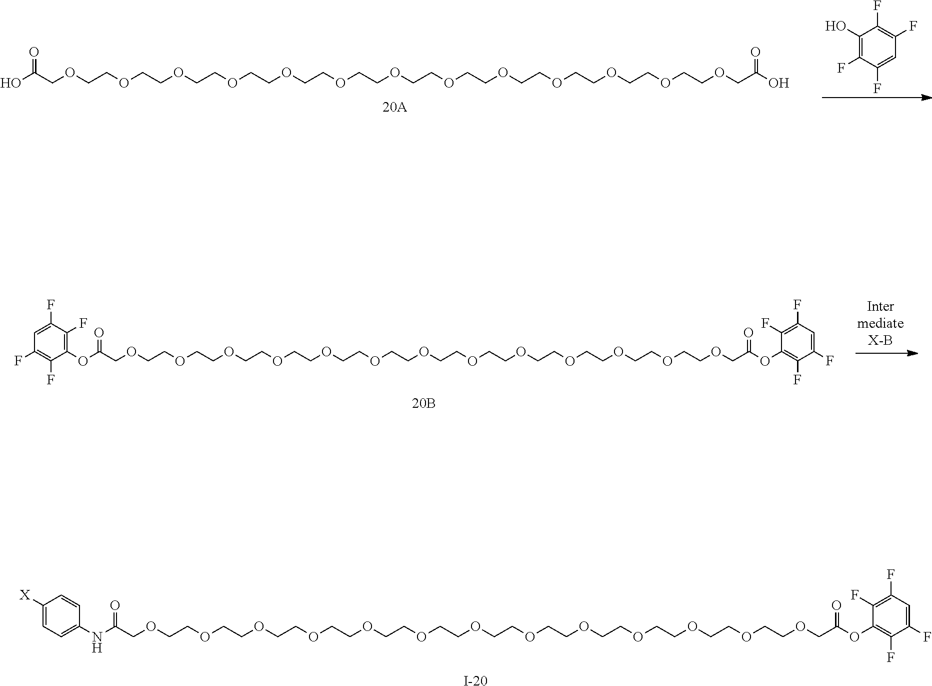

Compound 20B is synthesized by employing the procedure described for Compound 1B using Compound 20A in lieu of Compound 1A.

Compound I-20 is synthesized by employing the procedure described for Compound 1 using Compound 20B and Intermediate X-B in lieu of Compound 1B and Intermediate A-10.

### Example 21

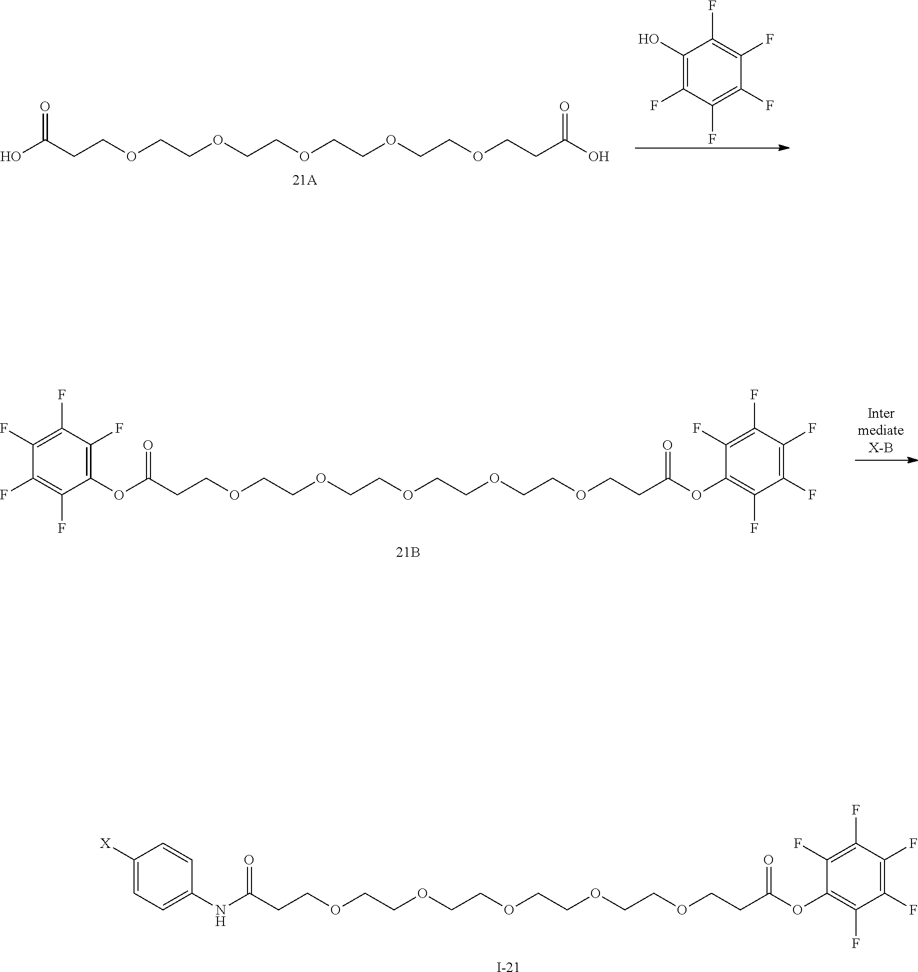

Compound 21B is synthesized by employing the procedure described for Compound 1B using Compound 21A and pentafluorophenol in lieu of Compound 1A and 2,3,5,6-tetrafluorophenol.

Compound I-21 is synthesized by employing the procedure described for Compound 1 using Compound 21B and Intermediate X-B in lieu of Compound 1B and Intermediate A-10.

### Example 22

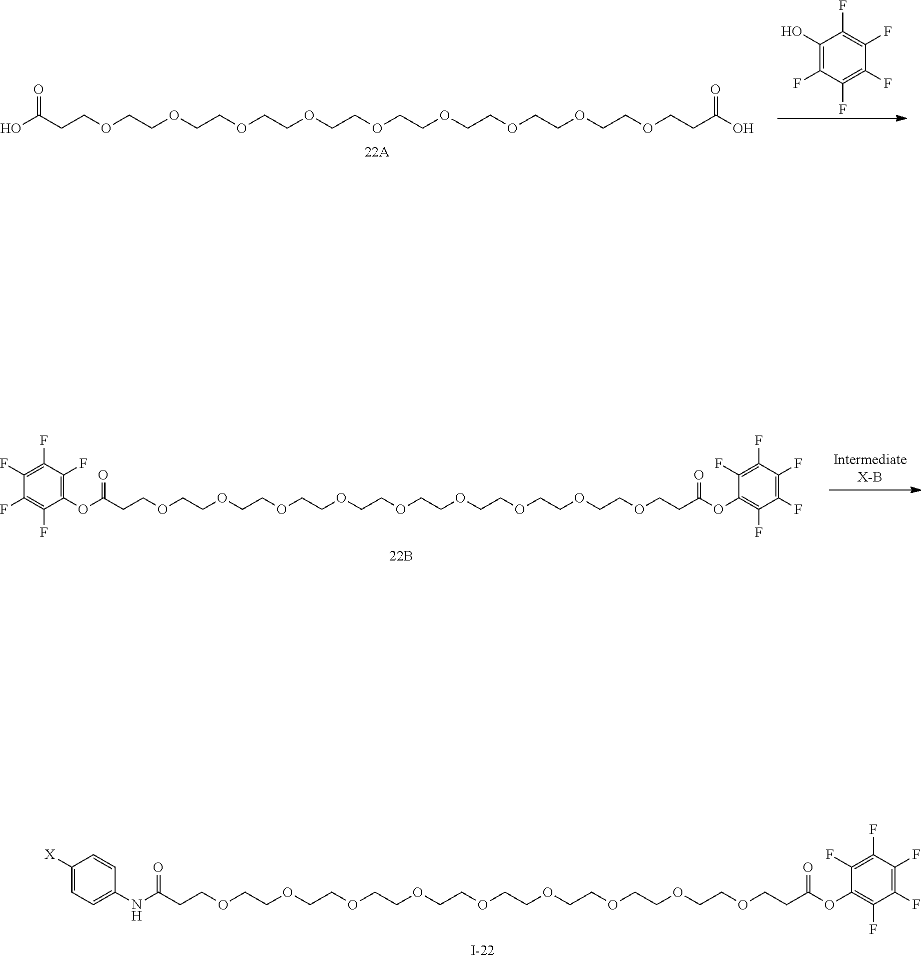

Compound 22B is synthesized by employing the procedure described for Compound 1B using Compound 22A and pentafluorophenol in lieu of Compound 1A and 2,3,5,6-tetrafluorophenol.

Compound I-22 is synthesized by employing the procedure described for Compound 1 using Compound 22B and Intermediate X-B in lieu of Compound 1B and Intermediate A-10.

### Example 23

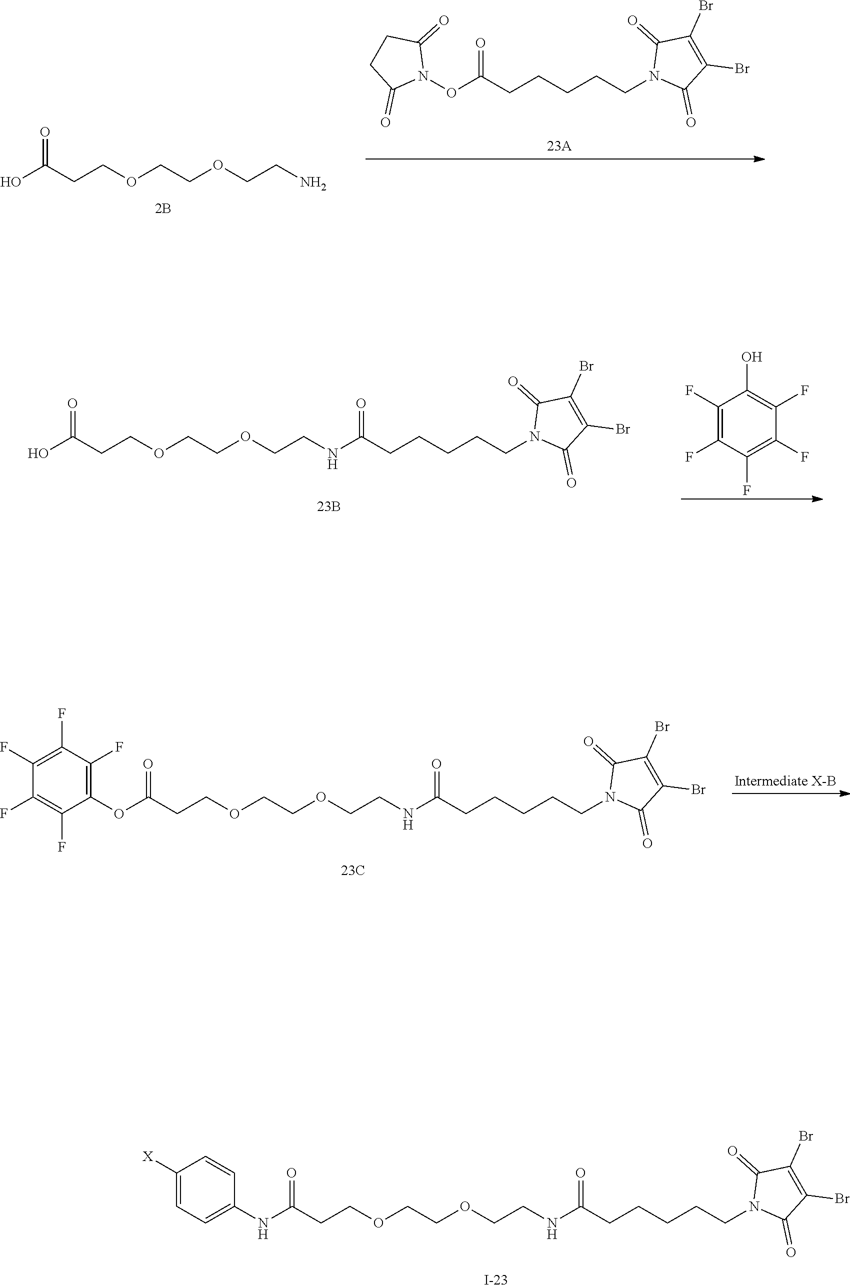

Compounds 23B and 23C are synthesized by employing the procedures described for Compound 2D and 2E using Compounds 23A and 23B in lieu of Compounds 2C and 2D.

Compound I-23 is synthesized by employing the procedure described for Compound 2 using Compound 23C and Intermediate X-B in lieu of Compound 2E and Intermediate A-10.

### Example 24

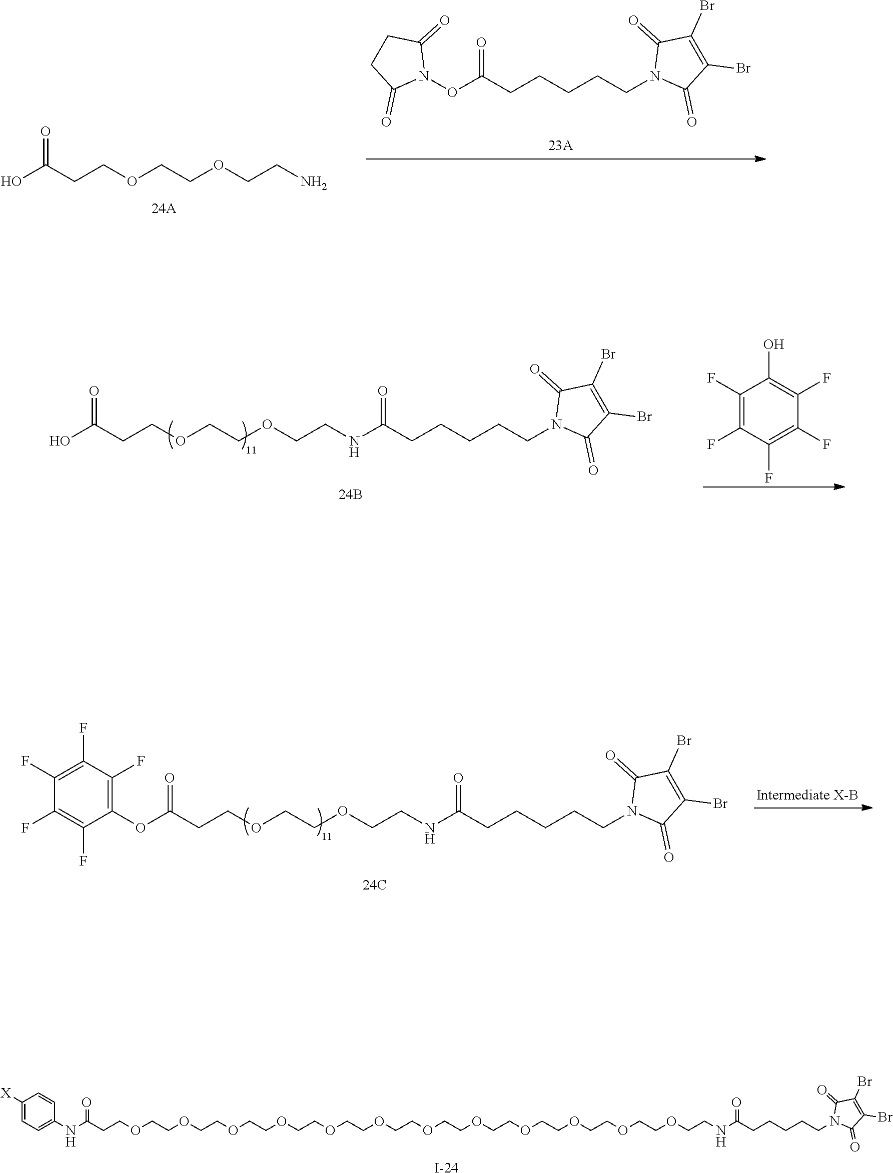

Compounds 24B and 24C are synthesized by employing the procedures described for Compound 2D and 2E using Compounds 24A, 23A and 24B in lieu of Compounds 2B, 2C and 2D.

Compound I-24 is synthesized by employing the procedure described for Compound 2 using Compound 24C and Intermediate X-B in lieu of Compound 2E and Intermediate A-10.

### Example 25

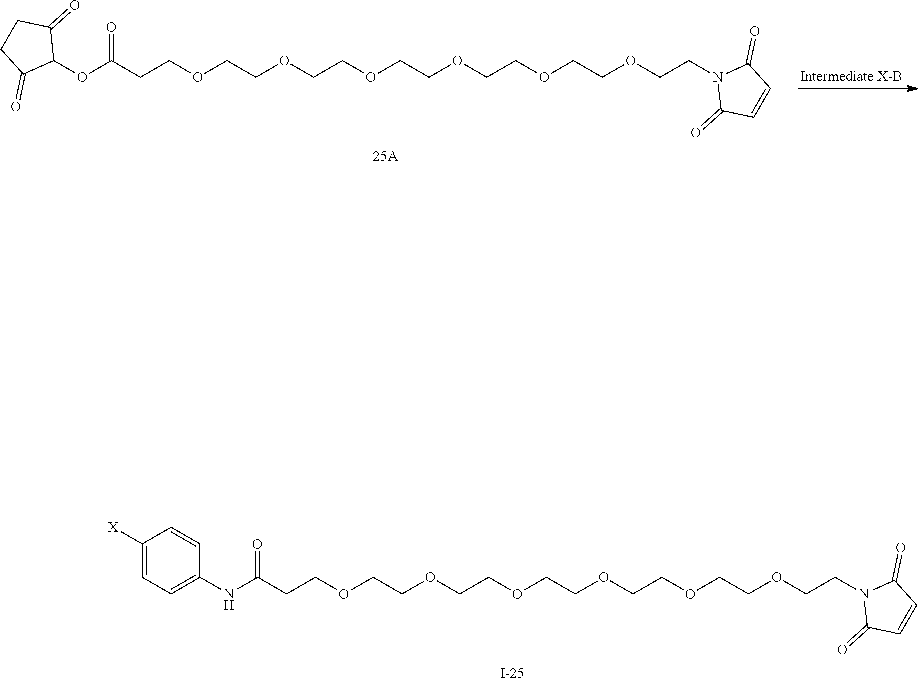

Compound I-25 is synthesized by employing the procedure described for Compound I-6 using Compound 25A and Intermediate X-B in lieu of Compound 6A and Intermediate A-10.

### Example 26

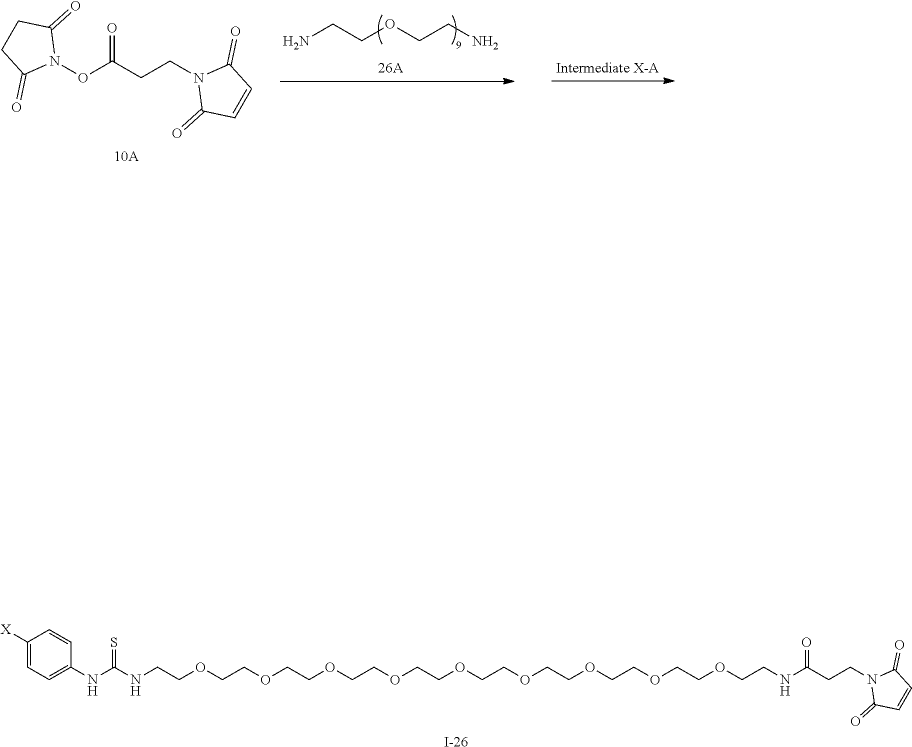

Compound I-26 is synthesized by employing the procedure described for Compound I-13 using Compound 26A and Intermediate X-A in lieu of Compounds 13A, 8D and tetrakis(acetonitrile)copper(I) hexafluorophosphate.

### Example 27

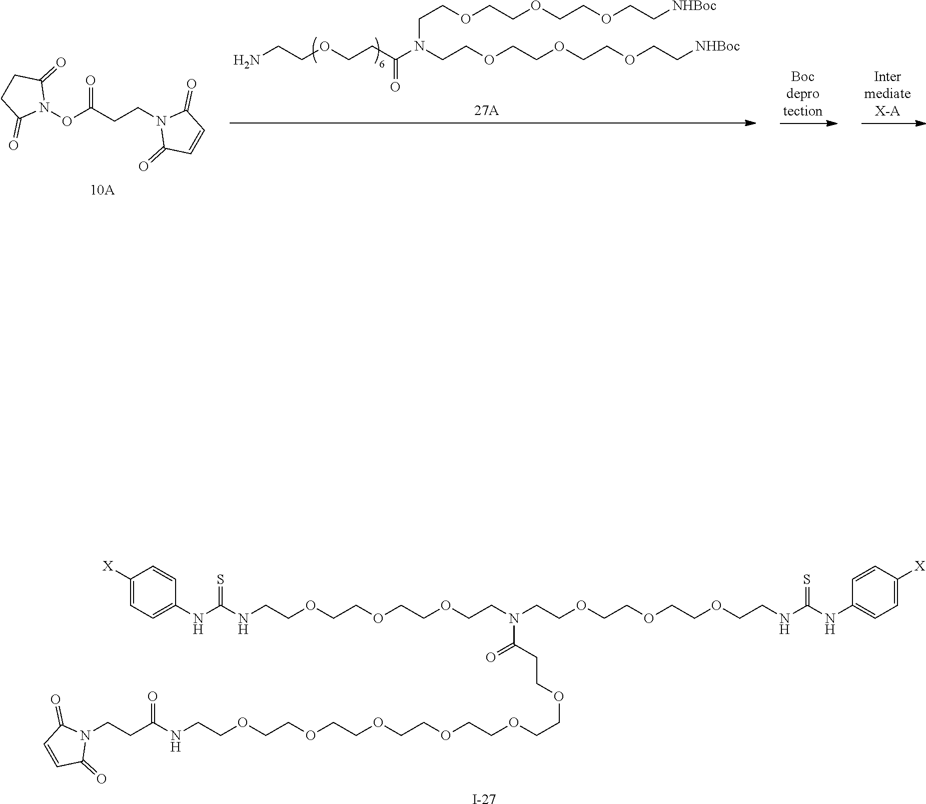

Compound I-27 is synthesized by employing the procedure described for Compound I-13 using Compound 27A and Intermediate X-A in lieu of Compounds 13A, 8D and tetrakis(acetonitrile)copper(I) hexafluorophosphate. A deprotection of the Boc protection group is performed under the standard Boc deprotection conditions before Intermediate X-A is added.

### Example 28

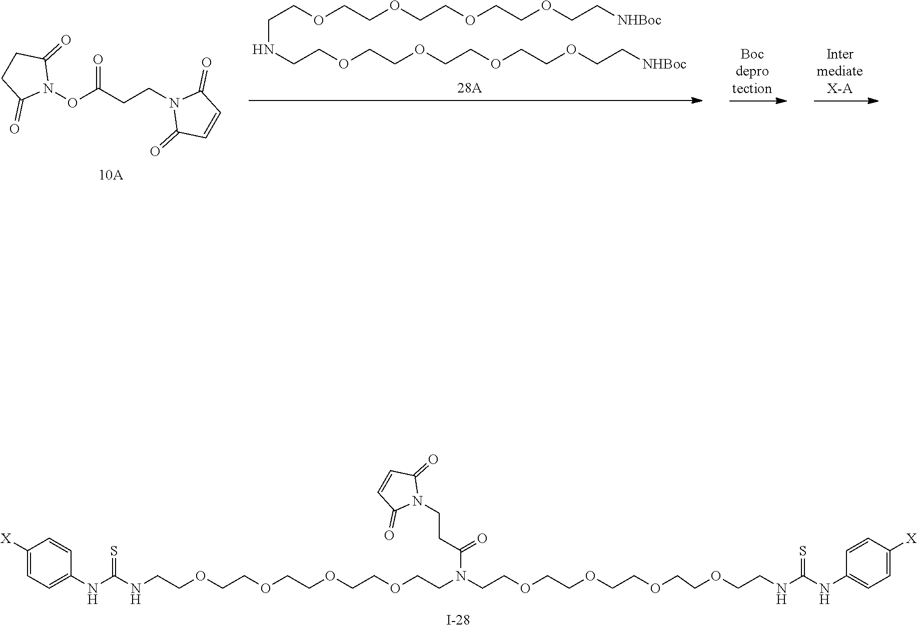

Compound I-28 is synthesized by employing the procedure described for Compound I-13 using Compound 28A and Intermediate X-A in lieu of Compounds 13A, 8D and tetrakis(acetonitrile)copper(I) hexafluorophosphate. A deprotection of the Boc protection group is performed under the standard Boc deprotection conditions before Intermediate X-A is added.

### Example 29

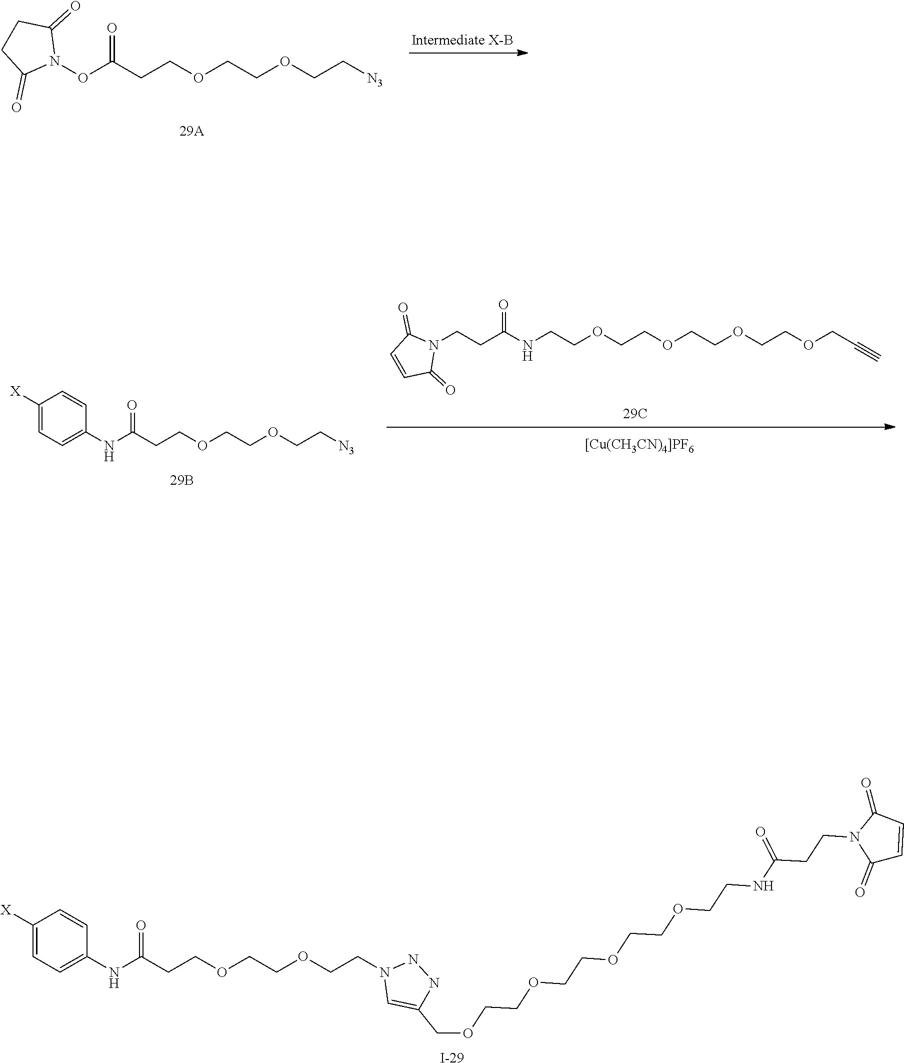

Compound 29B is synthesized by employing the procedure described for Compound 5B using Compound 29A and Intermediate X-B in lieu of Compound 5A and Intermediate A-10.

Compound I-29 is synthesized by employing the procedure described for Compound I-5 using Compounds 29B and 29C in lieu of Compounds 5B and 5C.

### Example 30

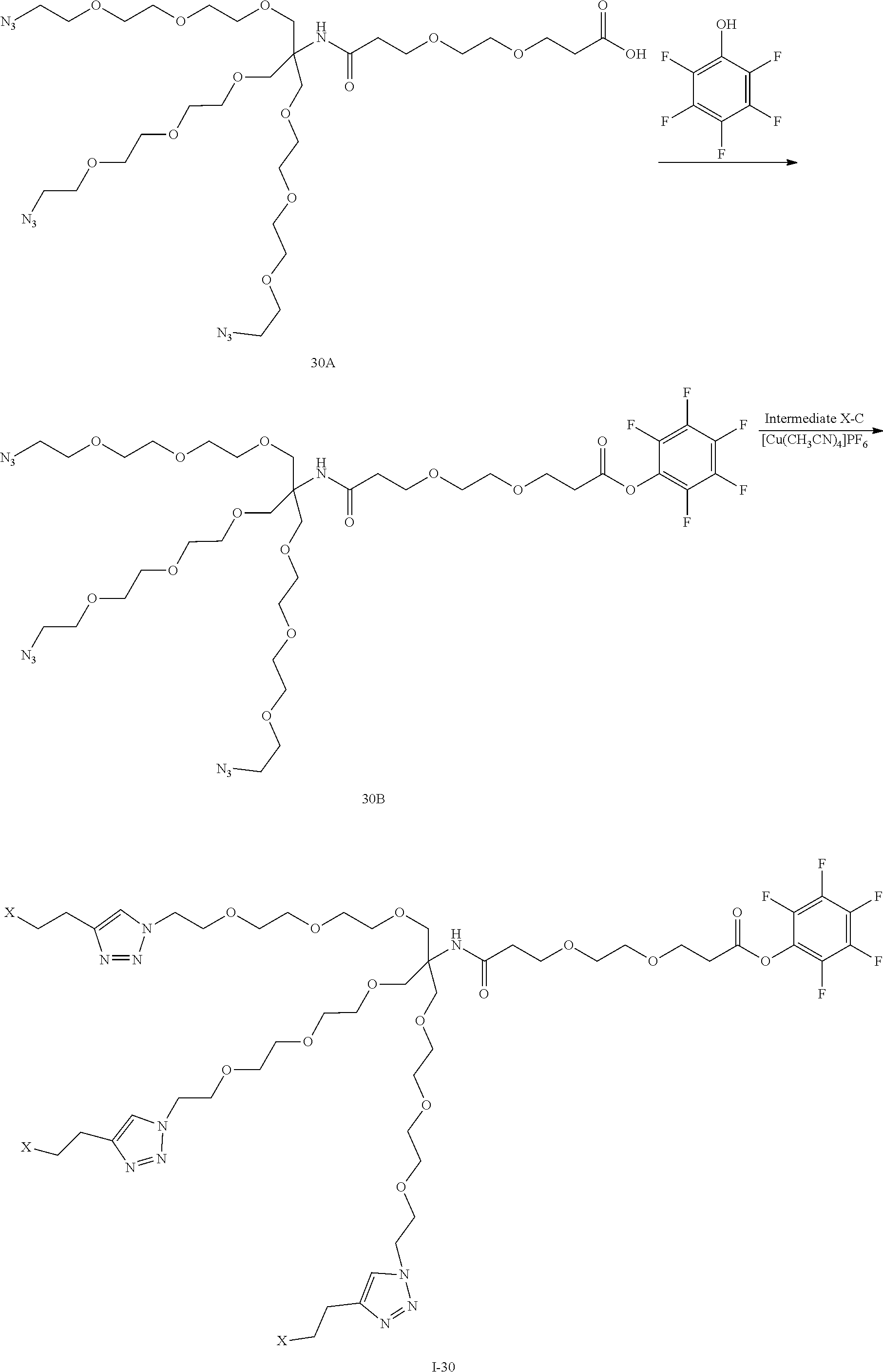

Compound 30B is synthesized by employing the procedure described for Compound 12B using Compound 30A in lieu of Compound 12A.

Compound I-30 is synthesized by employing the procedure described for Compound I-12 using Compound 30B and Intermediate X-C in lieu of Compounds 12B and 8D.

### Example 31

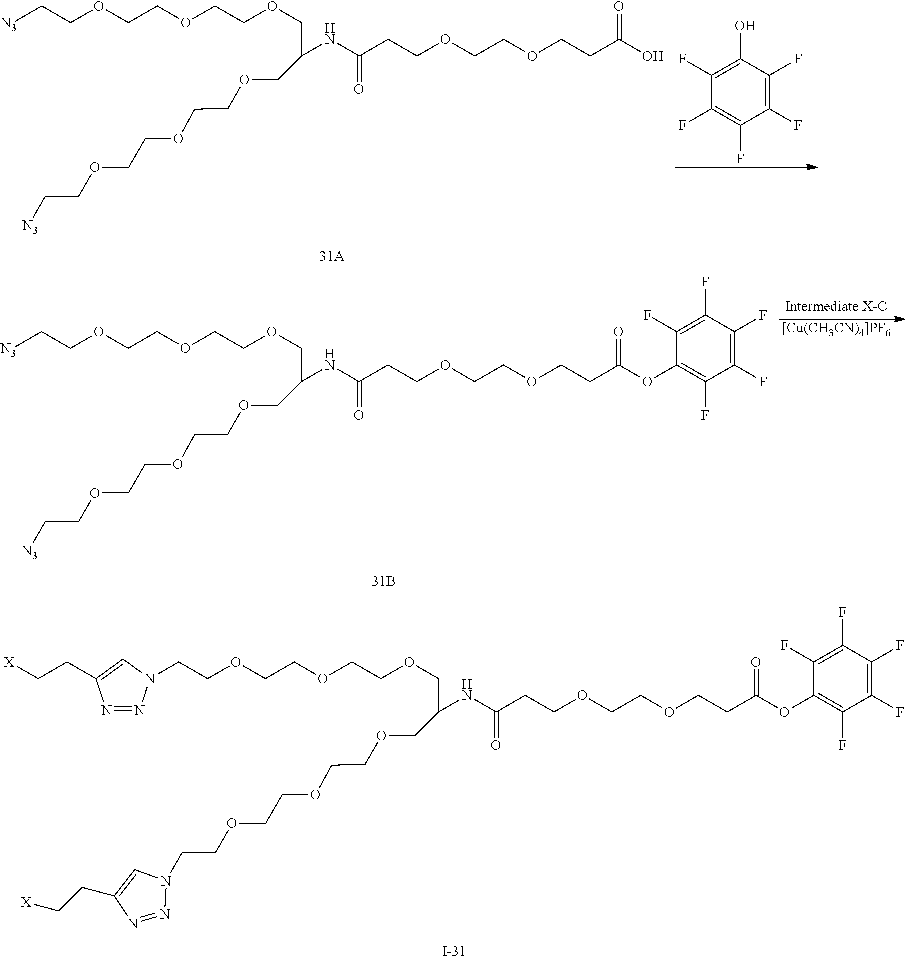

Compound 31B is synthesized by employing the procedure described for Compound 12B using Compound 31A in lieu of Compound 12A.

Compound I-31 is synthesized by employing the procedure described for Compound I-12 using Compound 31B and Intermediate X-C in lieu of Compounds 12B and 8D.

### Example 32

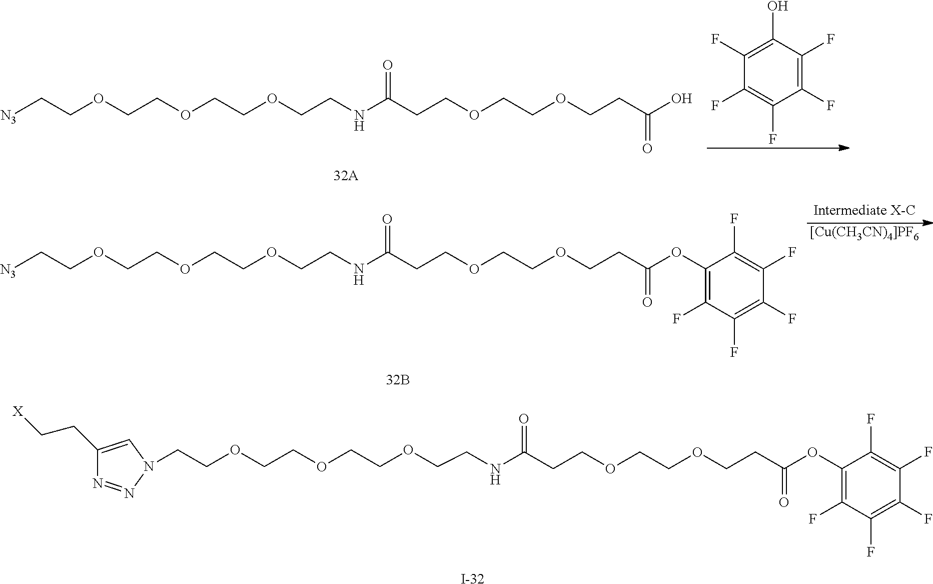

Compound 32B is synthesized by employing the procedure described for Compound 12B using Compound 32A in lieu of Compound 12A.

Compound I-32 is synthesized by employing the procedure described for Compound I-12 using Compound 32B and Intermediate X-C in lieu of Compounds 12B and 8D.

### Example 33: Synthesis of Compound I-33

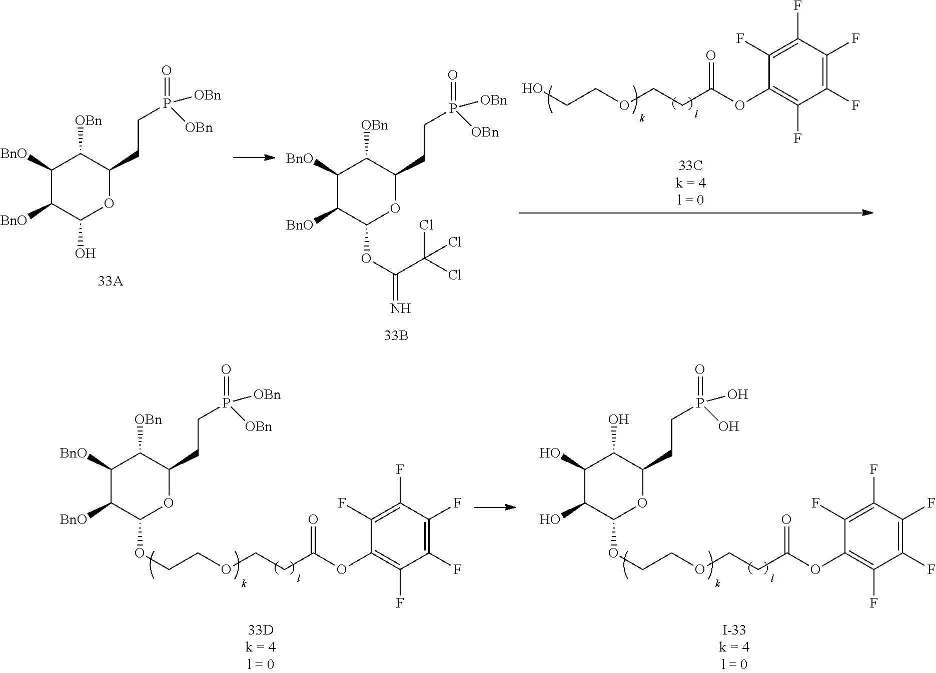

DBU (0.1 eq) is added to a stirred solution of dibenzyl (2-((2R,3R,4S,5S,6S)-3,4,5-tris(benzyloxy)-6-hydroxytetrahydro-2H-pyran-2-yl)ethyl)phosphonate (33A) (1.00 eq) and trichloroacetonitrile (10.0 eq) in DCM at 0° C. under nitrogen. The resulting mixture is stirred at 0° C. under nitrogen until LC-MS indicates complete conversion to Compound 33B. Most of the solvent is removed on a rotary evaporator. The residue is purified via silica gel chromatography to afford Compound 33B. Compound 33B (1.00 eq) is dissolved in dry DCM with stirring under nitrogen. Perfluorophenyl 14-hydroxy-3,6,9,12-tetraoxatetradecanoate (33C) (2.00 eq) is added and the resulting mixture is cooled to −78° C. with stirring under nitrogen. A solution of boron trifluoride diethyl etherate (0.500 eq) in dichloromethane is added slowly. The −78° C. cold bath is removed and the reaction mixture is allowed to slowly warm to 0° C. under nitrogen and then worked up. The crude material is purified via silica gel chromatography to afford Compound 33D. Compound 33D (1 eq) is dissolved with stirring in dry ethyl acetate. Palladium on carbon (0.05 eq) is added and the resulting mixture is stirred vigorously under a hydrogen balloon until LC-MS indicates complete conversion to Compound I-33. The resulting mixture is filtered through Celite, concentrated on a rotary evaporator, and purified via reverse phase preparatory HPLC to afford Compound I-33.

### Example 34: Synthesis of Compound I-34

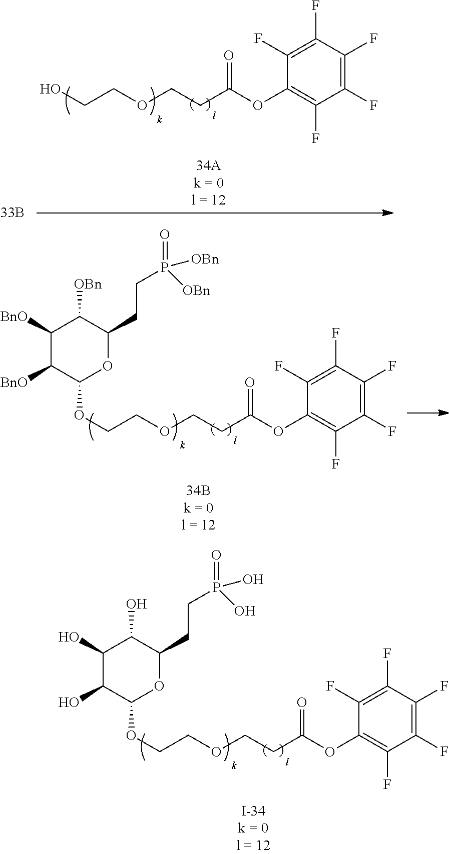

Compound 33B (1.00 eq) is dissolved in dry DCM with stirring under nitrogen. Perfluorophenyl 14-hydroxytetradecanoate (34A) (2.00 eq) is added and the resulting mixture is cooled to −78° C. with stirring under nitrogen. A solution of boron trifluoride diethyl etherate (0.500 eq) in dichloromethane is added slowly. The −78° C. cold bath is removed and the reaction mixture is allowed to slowly warm to 0° C. under nitrogen and then worked up. The crude material is purified via silica gel chromatography to afford Compound 34B. Compound 34B (1 eq) is dissolved with stirring in dry ethyl acetate. Palladium on carbon (0.05 eq) is added and the resulting mixture is stirred vigorously under a hydrogen balloon until LC-MS indicates complete conversion to Compound I-34. The resulting mixture is filtered through Celite, concentrated on a rotary evaporator, and purified via reverse phase preparatory HPLC to afford Compound I-34.

### Example 35: Synthesis of Compound I-35

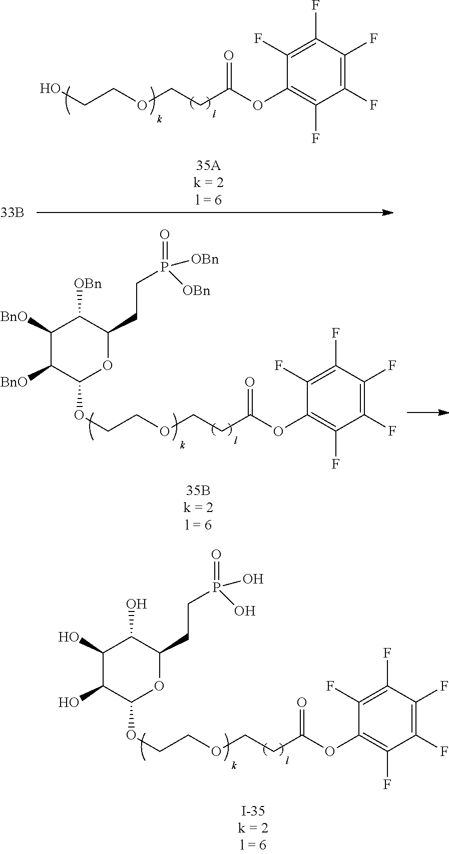

Compound 33B (1.00 eq) is dissolved in dry DCM with stirring under nitrogen. Perfluorophenyl 8-(2-(2-hydroxyethoxy)ethoxy)octanoate (35A) (2.00 eq) is added and the resulting mixture is cooled to −78° C. with stirring under nitrogen. A solution of boron trifluoride diethyl etherate (0.500 eq) in dichloromethane is added slowly. The −78° C. cold bath is removed and the reaction mixture is allowed to slowly warm to 0° C. under nitrogen and then worked up. The crude material is purified via silica gel chromatography to afford Compound 35B. Compound 35B (1 eq) is dissolved with stirring in dry ethyl acetate. Palladium on carbon (0.05 eq) is added and the resulting mixture is stirred vigorously under a hydrogen balloon until LC-MS indicates complete conversion to Compound I-35. The resulting mixture is filtered through Celite, concentrated on a rotary evaporator, and purified via reverse phase preparatory HPLC to afford Compound I-35.

### Example 36: Synthesis of Compound B

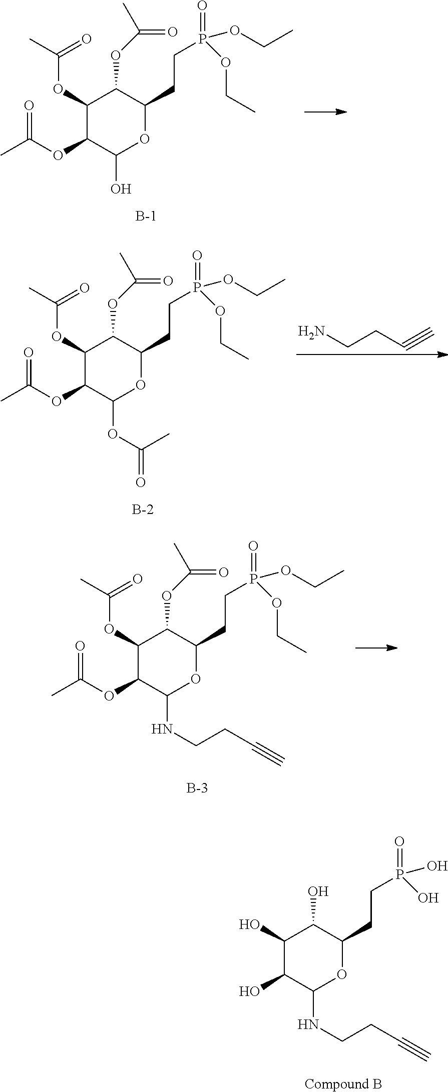

Compound B is synthesized employing the procedures described for Compound 8D using but-3-yn-1-amine in lieu of but-3-yn-1-ol.

Alternatively, Intermediate B-2 may be prepared by addition of pyridine to a solution of Intermediate B-1 in excess acetic anhydride. The resulting mixture is stirred at 20° C. for 16 h. The reaction solution is concentrated in vacuo and the residual pyridine is removed by azeotropic distillation with toluene followed by high vacuum drying to afford Intermediate B-2.

### Example 37: Synthesis of Compound I-37

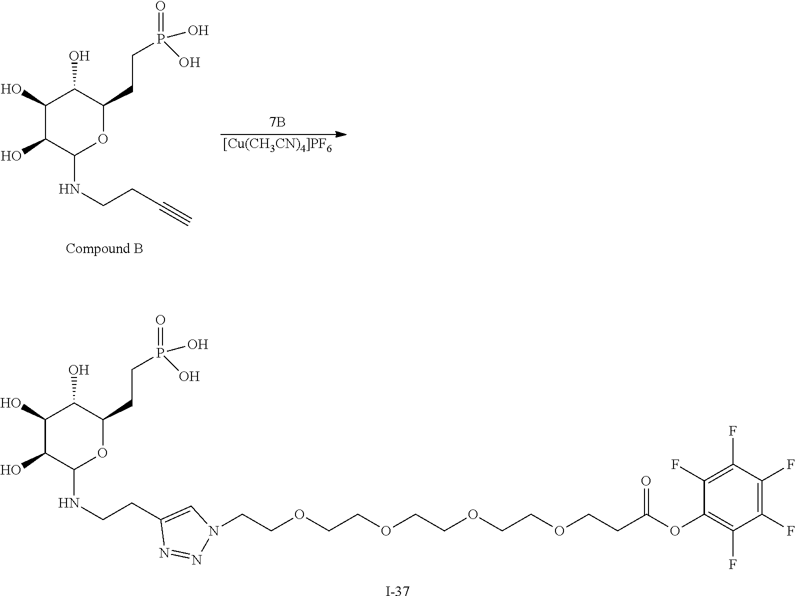

Compound I-37 is synthesized employing the procedures described for Compound I-8 using Compound B in lieu of Compound 8D.

### Example 38: Synthesis of Compound I-38

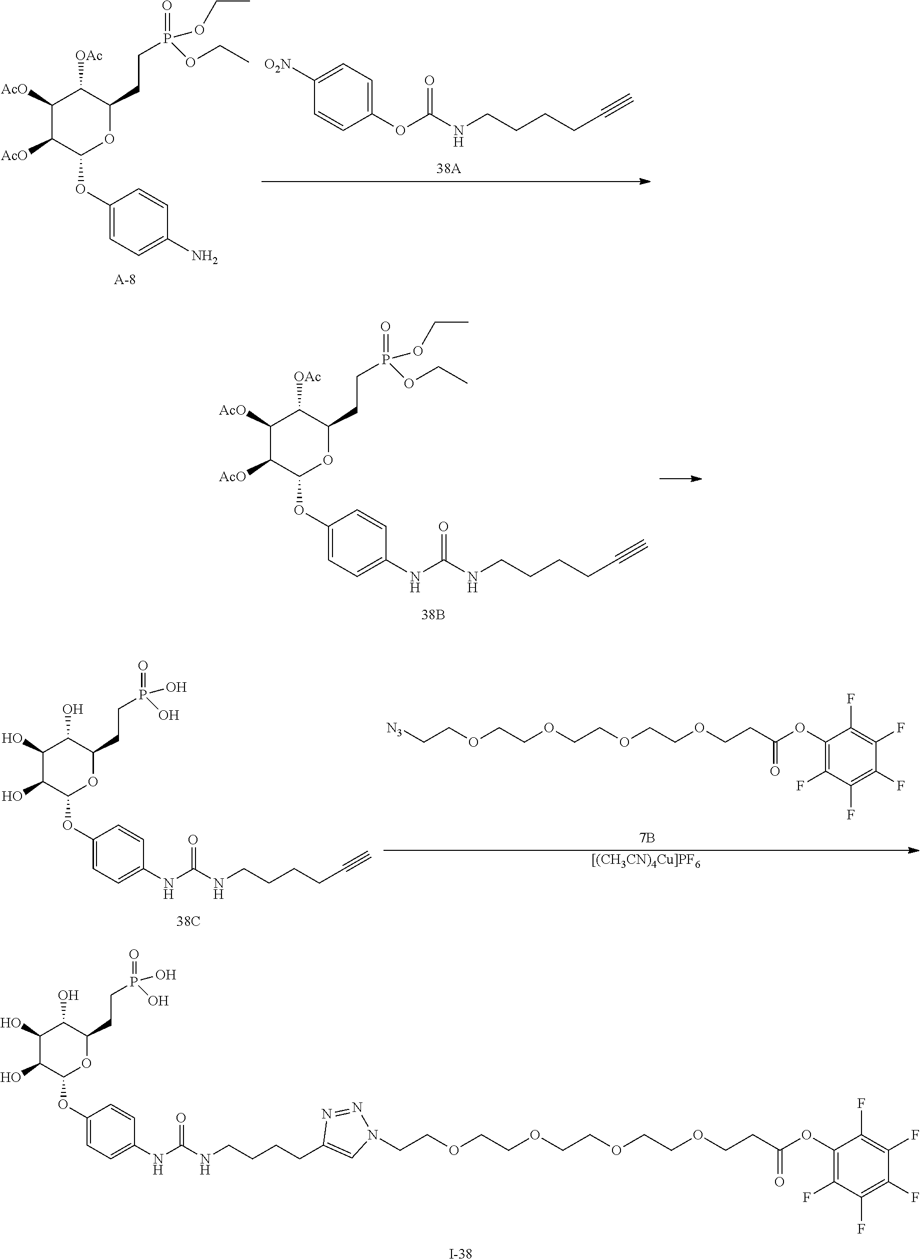

To a round bottom flask containing Intermediate A-8 (1.00 eq, 218 mg, 0.398 mmol) was added (4-nitrophenyl)N-hex-5-ynylcarbamate (38A) (1.80 eq, 188 mg, 0.717 mmol) and anhydrous DCM (4 mL). To the reaction solution was added triethylamine (2.08 eq, 0.11 mL, 0.826 mmol) and the solution was allowed to stir at 40° C. for 16 hr. The reaction mixture was then diluted with dichloromethane (30 mL) and washed with aq. NaOH, water, and brine. The organic layer was dried over anhydrous MgSO₄, filtered, and concentrated in vacuo. The residue was purified by column chromatography on silica gel, eluting with methanol/chloroform to afford Compound 38B. Yield: 154 mg, 58%); LCMS m/z 655.6 [M+1]⁺.

To a nitrogen-purged round bottom flask containing Compound 38B (1.00 eq, 170 mg, 0.260 mmol) was added acetonitrile (4 mL). The solution was allowed to cool to 0° C. under nitrogen prior to dropwise addition of TMSBr (5.00 eq, 0.18 mL, 1.30 mmol). The cold bath was removed and the resulting mixture was stirred at room temperature under nitrogen. LCMS at 2 h shows no SM remaining and product M+H=599.6 observed. The solvent was removed on a rotary evaporator and the residue was dried under high vacuum. The resulting intermediate, 2-[(2R,3R,4S,5S,6R)-3,4,5-triacetoxy-6-[4-(hex-5-ynylcarbamoylamino)phenoxy]tetrahydropyran-2-yl]ethylphosphonic acid (155 mg, 0.259 mmol, 99.72% yield), was dissolved in methanol (3 mL). To the stirring solution under nitrogen was added NaOMe, 25 wt % in MeOH (2.50 eq, 0.14 mL, 0.649 mmol). The resulting mixture was stirred at room temperature under nitrogen for 50 min. LCMS found mostly starting material remains. Another aliquot of NaOMe, 25 wt % in MeOH (2.50 eq, 0.14 mL, 0.649 mmol) was added and allowed to stir at 20° C. for 1 hr longer. Acetic acid (13.5 eq, 0.20 mL, 3.50 mmol) was added, and solvents were removed on a rotary evaporator. The residue was taken up in DMSO and purified via preparatory HPLC (0-35% acetonitrile in water with 0.1% TFA). The purified product fractions were combined and lyophilized to dryness to afford Compound 38C as a white solid. Yield: 45 mg, 37%; LCMS m/z 473.6 [M+1]+.

To a nitrogen-purged glass vial was Compound 38C (1.00 eq, 19.0 mg, 0.0402 mmol) with a stirring bar. To the vial was added a solution of Compound 7B (1.20 eq, 22.1 mg, 0.0483 mmol) in NMP (1 mL) followed by [(CH₃CN)₄Cu]PF₆ (2.50 eq, 37.5 mg, 0.101 mmol). The resulting clear yellow solution was capped and stirred at room temperature for 30 min. LCMS analysis found reaction to be complete. The reaction mixture was diluted with mixture of NMP, ethanol, and acetic acid, filtered, and purified via preparatory HPLC (15-65% acetonitrile in water with 0.1% TFA) over a 30 min run. Fractions containing the desired product were combined and lyophilized to dryness to afford Compound I-38 as a white solid. Yield: 12 mg, 37%; LCMS m/z 930.5 [M+1]⁺; ¹H NMR (300 MHz, DMSO-d6 D₂O) δ 7.77 (s, 1H), 7.24 (d, J=8.5 Hz, 2H), 6.88 (d, J=8.6 Hz, 2H), 5.23 (s, 1H), 4.42 (t, J=5.1 Hz, 2H), 3.89-3.25 (m, 20H), 3.05 (t, J=6.2 Hz, 2H), 2.95 (t, J=5.8 Hz, 2H), 2.59 (t, J=7.5 Hz, 2H), 2.02-1.82 (m, 1H), 1.70-1.33 (m, 6H), 1.30-1.05 (m, 1H).

### Example 39: Synthesis of Compound I-39

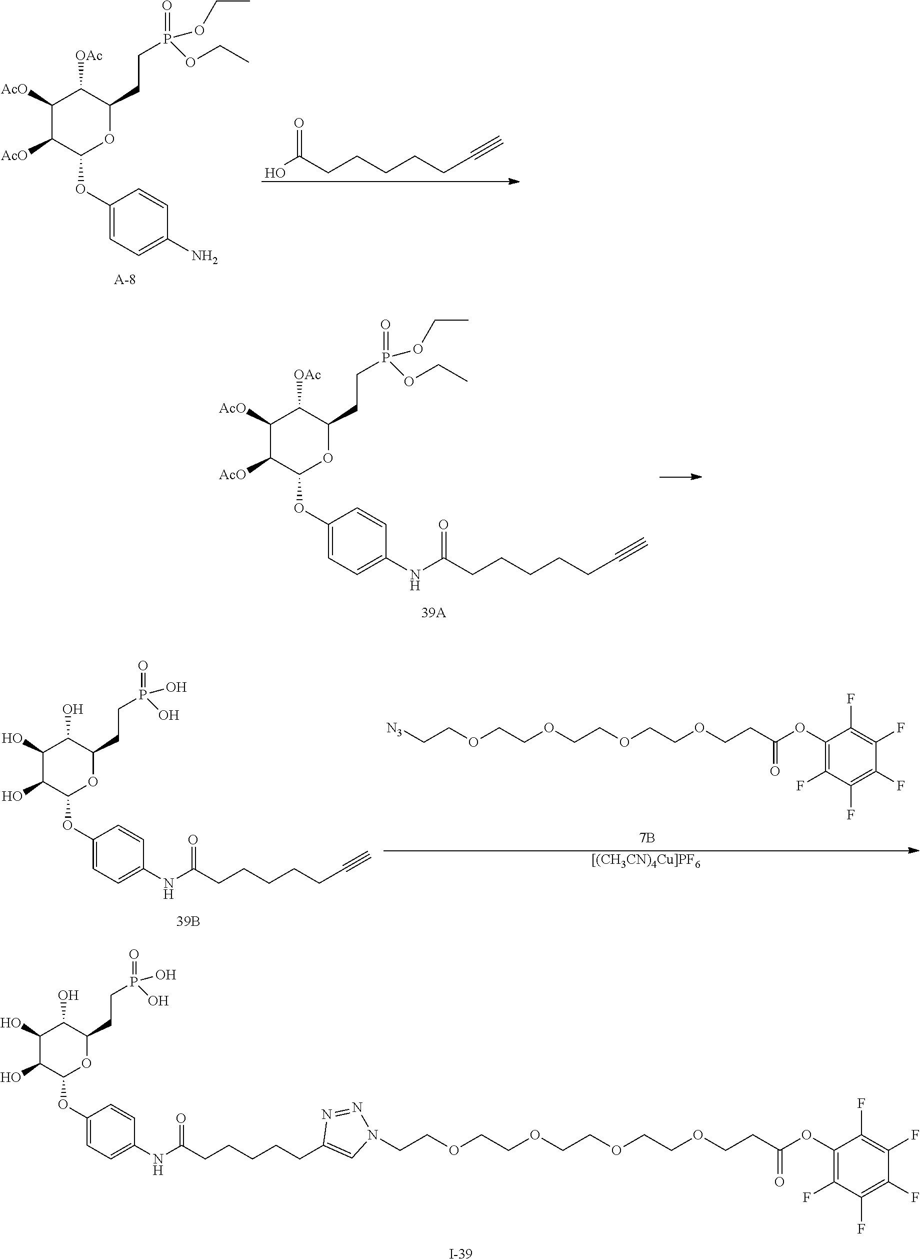

To a nitrogen-purged round bottom flask was added oct-7-ynoic acid (1.66 eq, 82.6 mg, 0.589 mmol), DMF (3 mL), and HATU (1.50 eq, 203 mg, 0.534 mmol). The reaction solution was allowed to stir at 20° C. for 20 min prior to the addition of Intermediate A-8 (1.00 eq, 195 mg, 0.356 mmol) in 1 mL of DMF. The reaction solution was allowed to stir 24 hr at 20° C. prior to analysis by LCMS. The reaction solution was diluted with EtOAc (30 mL) and washed with aq. Sat. NH₄Cl (20 mL) and then aq. sat. NaCl (20 mL). The partitioned EtOAc phase was dried over Na₂SO₄, filtered, and concentrated in vacuo to afford crude product that purified by column chromatography on silica gel using a mobile phase of 100% Hx to 75% EtOAc/Hx over 15 min to afford Compound 39A. Yield: 182 mg, 76%; LCMS m/z 653.6 [M+1]+.

To a nitrogen-purged round bottom flask containing Compound 39A (1.00 eq, 182 mg, 0.278 mmol) and anhydrous acetonitrile (1 mL) at 0° C. was added TMSBr (5.00 eq, 0.18 mL, 1.39 mmol) under nitrogen. The cold bath was removed and the resulting mixture was stirred at room temperature under nitrogen for 3.5 h. LCMS analysis shows no starting reagent remaining. Volatiles were removed on a rotary evaporator and the residue was dried under high vacuum briefly. The residue was dissolved in methanol (1 mL) with stirring under nitrogen and sodium methoxide, 25 wt % in MeOH (2.50 eq, 0.15 mL, 0.696 mmol) was added. The resulting mixture was stirred at room temperature under nitrogen for 30 min. To the reaction mixture was added Acetic Acid (5.00 eq, 0.080 mL, 1.39 mmol), and the volatiles were removed in vacuo. The residue was taken up in DMSO and purified via reverse-phase preparatory HPLC (0-35% acetonitrile in water with 0.1% TFA) to afford purified fractions. The combined fractions were lyophilized to dryness to afford Compound 39B as a white solid. Yield: 65 mg, 50%; LCMS m/z 472.3 [M+1]+

To a nitrogen-purged glass vial equipped with magnetic stir bar was added Compound 39B. To the vial was added a solution of Compound 7B (1.20 eq, 34.9 mg, 0.0764 mmol) in NMP (1 mL) followed by [(CH₃CN)₄Cu]PF₆ (2.50 eq, 59.3 mg, 0.159 mmol). The resulting clear yellow solution was capped and stirred at room temperature for 30 min. LCMS analysis found no starting material remaining. The reaction mixture was diluted with NMP (0.3 mL), ethanol (0.3 mL), and acetic acid (0.3 mL), filtered, and purified via reverse-phase preparatory HPLC (15-65% acetonitrile in water with 0.1% TFA) to afford purified fractions. Fractions containing the desired product were combined and lyophilized to dryness to afford Compound I-39 as a white solid. Yield: 55 mg, 59%; LCMS m/z 929.6 [M+1]⁺; ¹H NMR (300 MHz, DMSO-d6 with D₂O) δ 7.76 (s, 1H), 7.46 (d, J=8.8 Hz, 2H), 6.94 (d, J=8.2 Hz, 2H), 5.28 (s, 1H), 4.41 (t, J=5.1 Hz, 2H), 3.86-2.87 (m, 22H), 2.64-2.53 (m, 2H), 2.23 (t, J=7.5 Hz, 2H), 1.99-1.80 (m, 1H), 1.68-1.40 (m, 6H), 1.37-1.05 (m, 3H).

### Example 40: Synthesis of Compound I-40

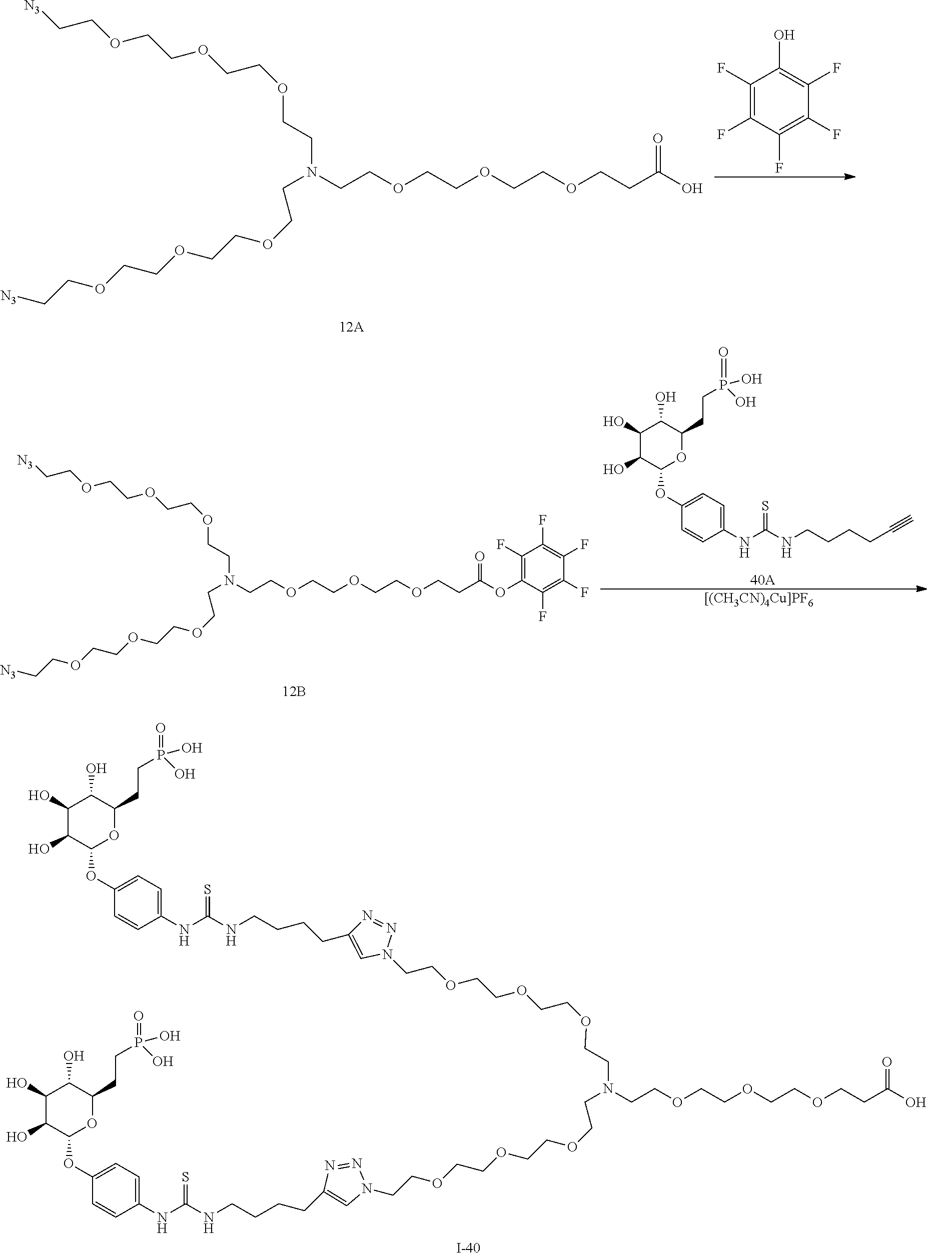

To a stirred solution of Compound 12A (1.00 eq, 500 mg, 0.802 mmol) in THF (2.5 mL) was added sequentially: DCC (1.50 eq, 248 mg, 1.20 mmol), a solution of 2,3,4,5,6-pentafluorophenol (1.70 eq, 251 mg, 1.36 mmol) in THF (1 mL), and then 4-dimethylaminopyridine (0.0300 eq, 2.9 mg, 0.0241 mmol). The resulting mixture was capped and stirred at rt for 17 h. The reaction mixture was diluted with Et₂O and filtered. The filtrate was concentrated on a rotary evaporator. The residue was taken up in DCM and purified via silica gel chromatography (0-100% acetonitrile in DCM) to afford Compound 12B as a yellow oil. Yield: 258 mg, 41%; LCMS m/z 790.7 [M+1]⁺; ¹H NMR (300 MHz, Chloroform-d) δ 3.87 (t, J=6.2 Hz, 2H), 3.74-3.56 (m, 16H), 3.39 (t, J=5.1 Hz, 4H), 2.94 (t, J=6.2 Hz, 2H).

A solution of Compound 40A (2.20 eq, 36.1 mg, 0.0738 mmol) in NMP (0.6 mL) was added to Compound 12B (1.00 eq, 26.5 mg, 0.0336 mmol) in a 1 dram vial with a stirbar. The resulting solution was stirred and [(CH₃CN)₄Cu]PF₆ (5.00 eq, 62.5 mg, 0.168 mmol) was added. The resulting light yellow solution was capped and stirred at room temperature for 25 min. The reaction mixture was diluted with a mixture of NMP, ethanol, and acetic acid, filtered, and purified via preparatory HPLC (15-40% acetonitrile in water with 0.1% TFA). Fractions containing the desired product were combined and lyophilized to dryness to afford Compound I-40 as a white solid. Yield: 38.9 mg, 66%; LCMS m/z 1765.9 [M−1]−; ¹H NMR (300 MHz, DMSO-d₆ with D₂O) δ 7.81 (s, 2H), 7.19 (d, J=8.5 Hz, 4H), 6.99 (d, J=8.8 Hz, 4H), 5.33 (s, 2H), 4.43 (t, J=5.2 Hz, 4H), 3.90-3.23 (m, 54H), 2.97 (t, J=5.8 Hz, 2H), 2.69-2.34 (m, 4H), 2.01-1.81 (m, 2H), 1.73-1.40 (m, 12H), 1.34-1.10 (m, 2H).

### Example 41: Synthesis of Compound I-41

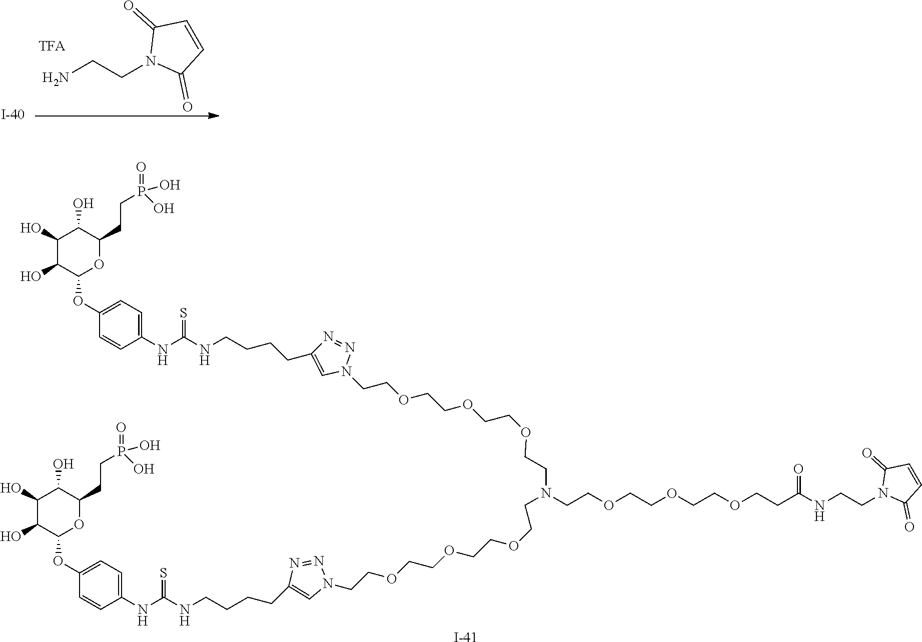

To Compound I-40 (1.00 eq, 32.7 mg, 0.0185 mmol) in a vial with a stirbar was added a solution of 1-(2-aminoethyl)-1H-pyrrole-2,5-dione TFA salt (1.15 eq, 5.4 mg, 0.0213 mmol) and DIPEA (3.00 eq, 0.0097 mL, 0.0555 mmol) in NMP (1 mL). The resulting clear slightly yellow solution was capped and stirred at room temperature for 30 min. The reaction mixture was diluted with acetic acid, filtered, and purified via preparatory HPLC (10-30% acetonitrile in water with 0.1% TFA). Fractions containing the desired product were combined and lyophilized to dryness to afford Compound I-41 as a slightly yellow solid. Yield: 18.5 mg, 58%; LCMS m/z 1722.0 [M−1]−; ¹H NMR (300 MHz, DMSO-d₆ with D₂O) δ 7.82 (s, 2H), 7.26-7.09 (m, 4H), 7.00 (d, J=8.5 Hz, 4H), 6.90 (s, 2H), 5.33 (s, 2H), 4.52-4.32 (m, 4H), 3.99-2.94 (m, 58H), 2.69-2.57 (m, 4H), 2.20 (t, J=6.5 Hz, 2H), 1.99-1.81 (m, 2H), 1.73-1.39 (m, 12H), 1.33-1.08 (m, 2H)

### Example 42: Synthesis of Compound I-42

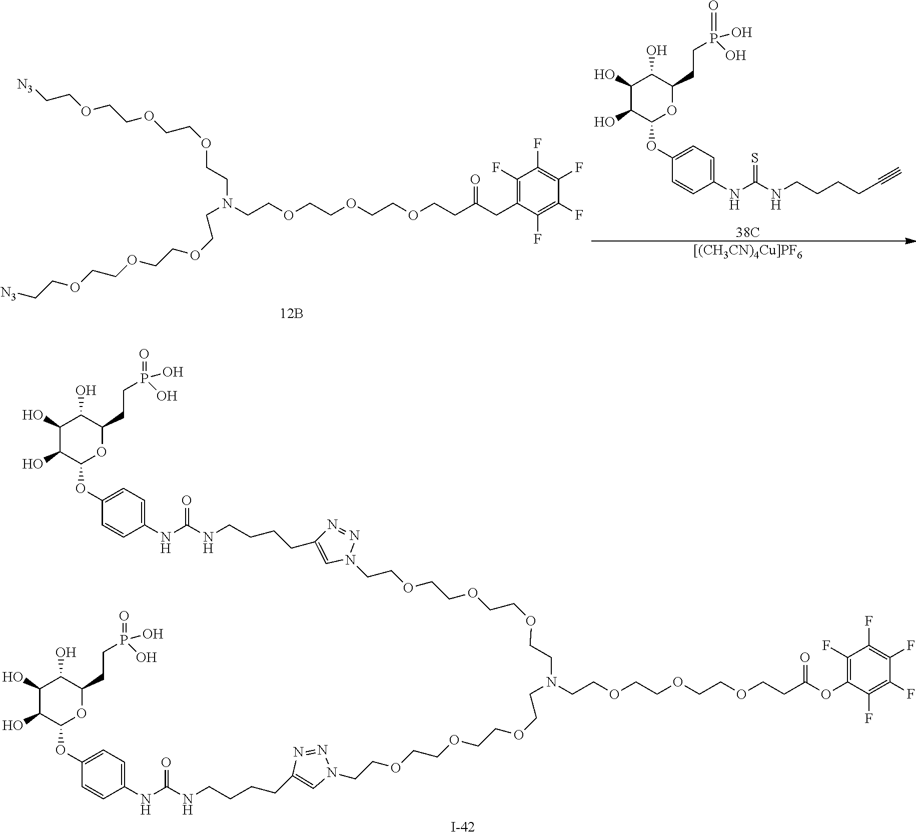

A solution of Compound 38C (2.00 eq, 35.9 mg, 0.0760 mmol) in NMP (0.4 mL) was added to Compound 12B (1.00 eq, 30.0 mg, 0.0380 mmol) in a 1 dram vial with a stirbar. The resulting solution was stirred and [(CH₃CN)₄Cu]PF₆ (5.00 eq, 70.8 mg, 0.190 mmol) was added. The resulting solution was capped and stirred at room temperature for 25 min. The reaction mixture was diluted with acetic acid, filtered, and purified via preparatory HPLC (15-40% acetonitrile in water with 0.1% TFA). Fractions containing the desired product were combined and lyophilized to dryness to afford Compound I-42 as a white solid. Yield: 40.0 mg, 61%; LCMS m/z 1734.0 [M−1]−; ¹H NMR (300 MHz, DMSO-d₆ with D₂O) δ 7.77 (s, 2H), 7.24 (d, J=8.5 Hz, 4H), 6.88 (d, J=8.6 Hz, 4H), 5.23 (s, 2H), 4.42 (t, J=5.1 Hz, 4H), 3.92-3.23 (m, 48H), 3.05 (t, J=6.2 Hz, 4H), 2.95 (t, J=5.8 Hz, 4H), 2.59 (t, J=7.5 Hz, 4H), 2.03-1.81 (m, 2H), 1.68-1.33 (m, 12H), 1.29-1.07 (m, 2H).

### Example 43: Synthesis of Compound I-43

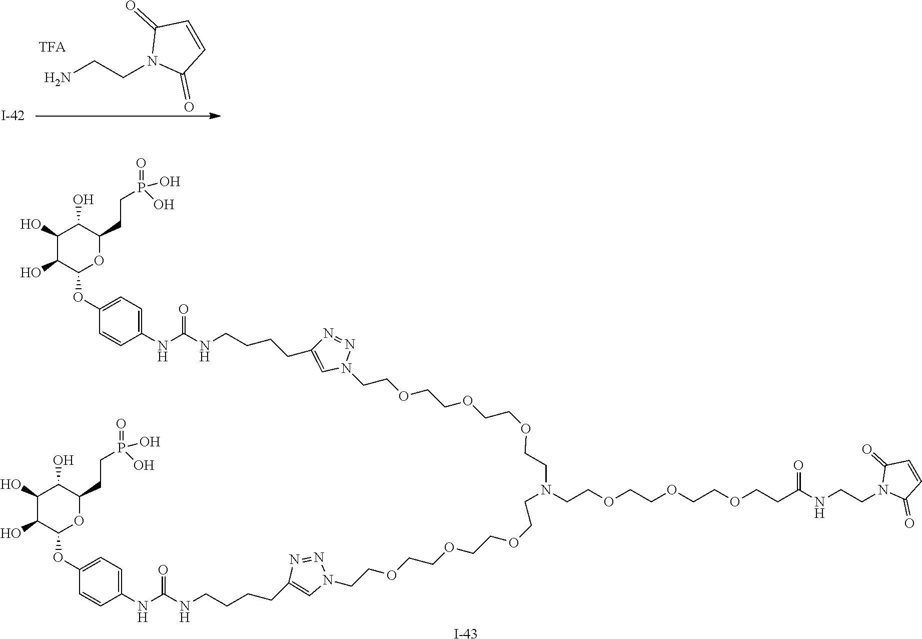

To Compound I-42 (1.00 eq, 26.1 mg, 0.0150 mmol) in a vial with a stirbar was added a solution of 1-(2-aminoethyl)-1H-pyrrole-2,5-dione TFA salt (1.15 eq, 4.4 mg, 0.0173 mmol) and DIPEA (3.00 eq, 0.0079 mL, 0.0451 mmol) in NMP (0.5 mL). The resulting solution was capped and stirred at room temperature for 30 min. The reaction mixture was diluted with acetic acid, filtered, and purified via preparatory HPLC (10-25% acetonitrile in water with 0.1% TFA). Fractions containing the desired product were combined and lyophilized to dryness to afford Compound I-43 as a white solid. Yield: 17.0 mg, 67%; LCMS m/z 1690.0 [M−1]−; ¹H NMR (300 MHz, DMSO-d₆ with D₂O) δ 7.76 (s, 2H), 7.23 (d, J=8.7 Hz, 4H), 6.97-6.80 (m, 6H), 5.24 (s, 2H), 4.48-4.33 (m, 4H), 4.04-2.95 (m, 58H), 2.59 (t, J=7.4 Hz, 4H), 2.19 (t, J=6.5 Hz, 2H), 2.01-1.81 (m, 2H), 1.69-1.32 (m, 12H), 1.32-1.07 (m, 2H).

### Example 44: Synthesis of Compound I-44

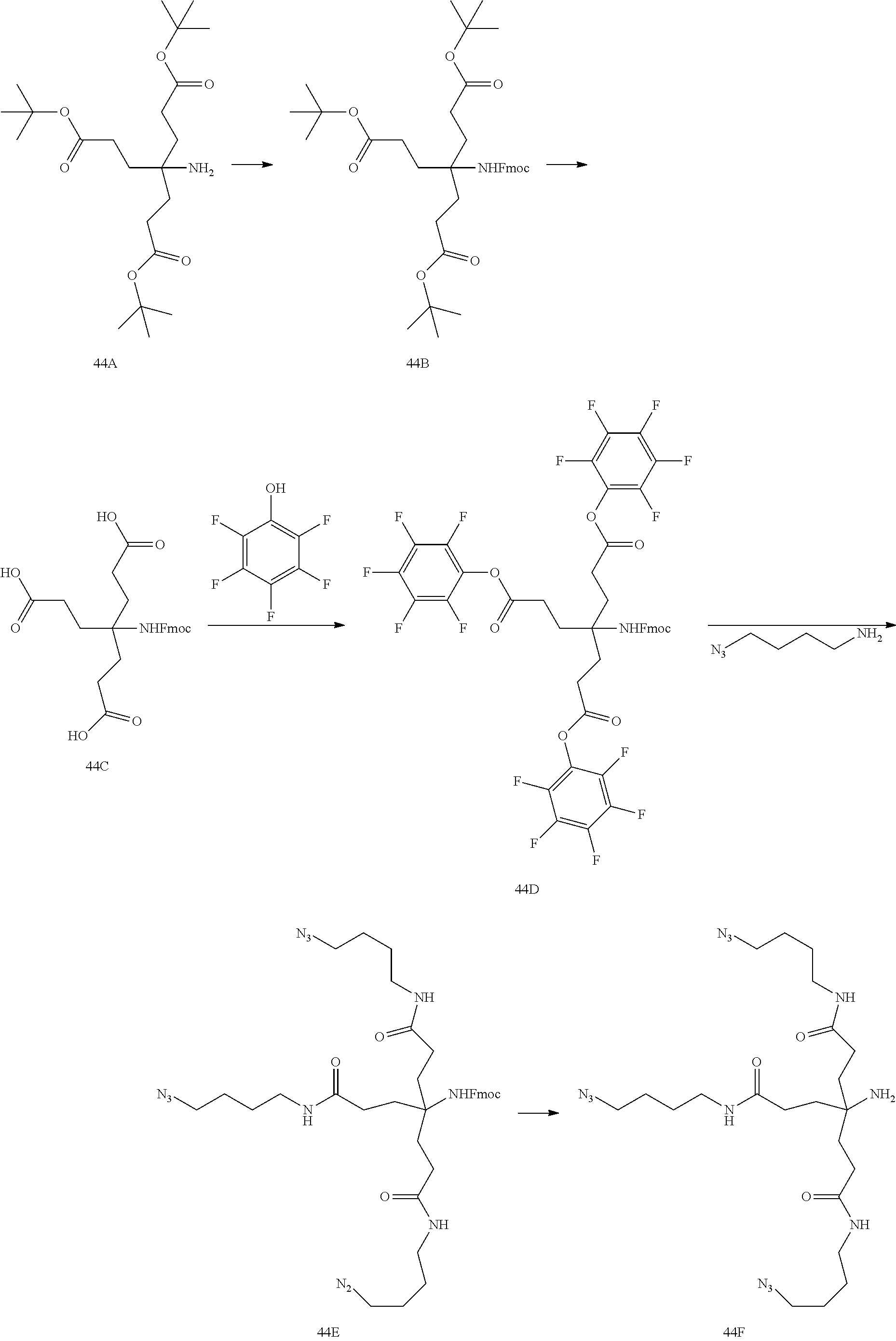

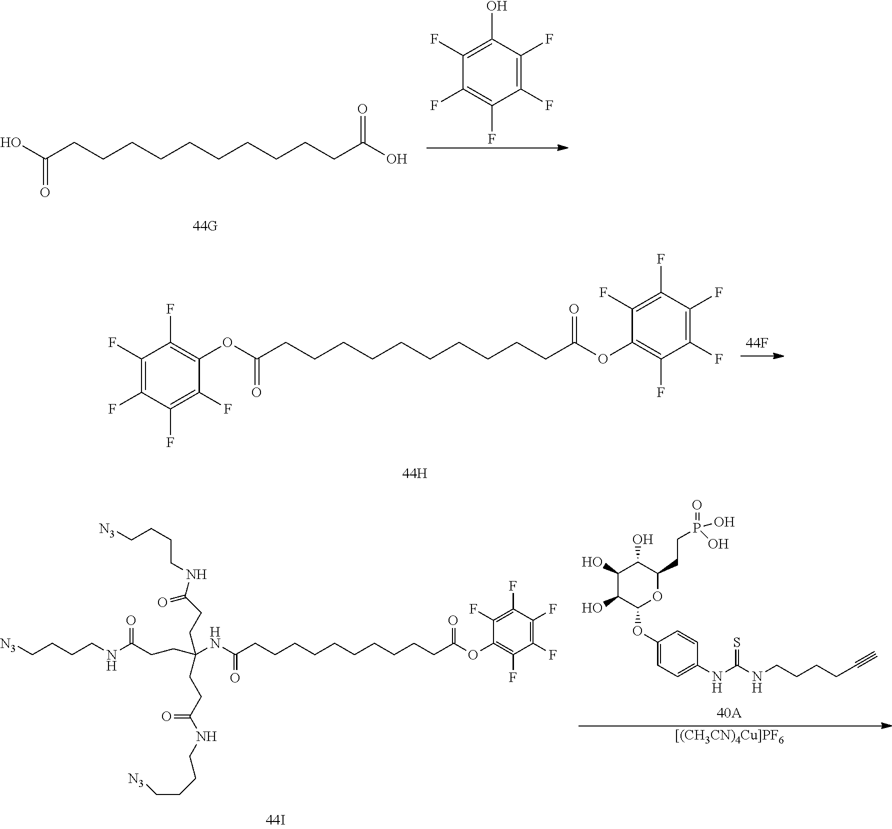

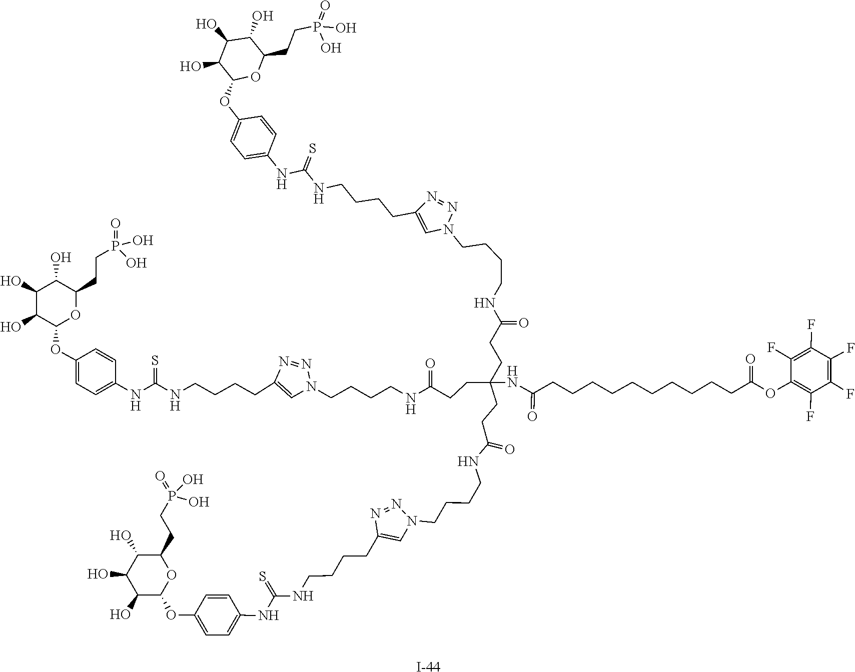

To a stirred mixture of di-tert-butyl 4-amino-4-(3-(tert-butoxy)-3-oxopropyl)heptanedioate (44A) (1.00 eq, 1.01 g, 2.43 mmol) in 1,4-dioxane (10 mL) at 0° C. was added 1 M sodium carbonate in water (1.50 eq, 3.6 mL, 3.65 mmol) and then a solution of FMOC-Cl (1.20 eq, 755 mg, 2.92 mmol) in 1,4-dioxane (4 mL). The cold bath was removed and the resulting mixture was stirred vigorously at room temperature for 2 h. The reaction mixture was partitioned between ethyl acetate and brine. The organics were dried over magnesium sulfate, filtered, concentrated on a rotary evaporator, and purified via silica gel chromatography (0-30% ethyl acetate in hexanes) to afford Compound 44B as a white foam-solid. Yield: 1.50 g, 97%; LCMS m/z 660.6 [M+Na]+; ¹H NMR (300 MHz, Chloroform-d) δ 7.76 (d, J=7.4 Hz, 2H), 7.59 (d, J=7.4 Hz, 2H), 7.40 (t, J=7.5 Hz, 2H), 7.31 (t, J=7.4 Hz, 2H), 5.01 (s, 1H), 4.36 (d, J=6.2 Hz, 2H), 4.18 (t, J=6.5 Hz, 1H), 2.25-2.12 (m, 6H), 1.98-1.83 (m, 6H), 1.43 (s, 27H).

To a stirred solution of Compound 44B (1.00 eq, 1.50 g, 2.35 mmol) in DCM (10 mL) at 0° C. was added water (0.5 mL) and then TFA (3 mL). The resulting mixture was allowed to warm to room temperature and then stirred at room temperature for 18 h. More TFA (2 mL) was added and stirring at room temperature was continued for another 26 h. Volatiles were removed on a rotary evaporator. The residue was concentrated to dryness twice from dry toluene and then dried under high vacuum to afford Compound 44C as a white solid. Yield: 1.19 g. LCMS 470.4 m/z [M+1]⁺; ¹H NMR (300 MHz, DMSO-d₆ with D₂O) δ 7.86 (d, J=7.5 Hz, 2H), 7.68 (d, J=7.5 Hz, 2H), 7.39 (t, J=7.4 Hz, 2H), 7.30 (t, J=7.9 Hz, 2H), 4.28-4.11 (m, 3H), 2.19-2.00 (m, 6H), 1.87-1.66 (m, 6H).

Compound 44C (1.00 eq, 549 mg, 1.17 mmol), 4-dimethylaminopyridine (0.0200 eq, 2.9 mg, 0.0234 mmol), DCC (3.30 eq, 796 mg, 3.86 mmol), pentafluorophenol (3.50 eq, 753 mg, 4.09 mmol), and DMF (2.5 mL) were combined in a scintillation vial with a stirbar, capped, and stirred at room temperature for 4 h. More DCC (482 mg, 2.34 mmol) and pentafluorophenol (430 mg, 2.34 mmol) in DMF (1 mL) was added and the resulting mixture was capped and stirred at room temperature for 2 h. The reaction mixture was diluted with diethyl ether and filtered. The filtrate was washed three times with brine, dried over magnesium sulfate, filtered, concentrated on a rotary evaporator, and purified via silica gel chromatography (0-50% ethyl acetate in hexanes) to afford Compound 44D and pentafluorophenol as a light yellow oil. Yield: 1.54 g. This material was taken on to the next step without further purification.

4-Azidobutan-1-amine (0.5 M in mTBE) (4.00 eq, 8.7 mL, 4.34 mmol) was added to a stirred solution of Compound 44D (1.00 eq, 1.50 g, 1.09 mmol) in THF (10 mL) at room temperature. The resulting clear solution was capped and stirred at room temperature for 2 h. Most of the volatiles were removed on a rotary evaporator at room temperature. The residue was loaded onto a silica gel loading column with DCM and purified via silica gel chromatography (0-100% ethyl acetate in DCM) then (0-10% methanol in DCM) to afford Compound 44E as a colorless waxy solid. Yield: 624 mg, 76%; LCMS m/z 758.6 [M+1]⁺; ¹H NMR (300 MHz, Chloroform-d) δ 7.77 (d, J=7.5 Hz, 2H), 7.60 (d, J=7.4 Hz, 2H), 7.41 (t, J=7.4 Hz, 2H), 7.31 (t, J=7.4 Hz, 2H), 6.08 (bs, 3H), 5.67 (bs, 1H), 4.37 (d, J=7.0 Hz, 2H), 4.18 (t, J=6.7 Hz, 1H), 3.34-3.13 (m, 12H), 2.24-2.09 (m, 6H), 2.04-1.85 (m, 6H), 1.66-1.47 (m, 12H).

Diethylamine (20.0 eq, 1.7 mL, 16.3 mmol) was added to a stirred solution of Compound 44E (1.00 eq, 619 mg, 0.817 mmol) in methanol (8 mL). The resulting clear solution was capped and stirred at room temperature for 16 h. Volatiles were removed on a rotary evaporator. Methanol (10 mL) was added and volatiles were removed on a rotary evaporator again. This was repeated again to drive off diethylamine. The residue was taken up in methanol and loaded onto a 5 g Strata X-C ion exchange column from Phenomenex. The column was eluted sequentially with acetonitrile, methanol, and then 5% ammonium hydroxide in methanol. Fractions containing the desired product were combined, concentrated on a rotary evaportor and dried under high vacuum to afford Compound 44F at 90% purity as a yellow oil. Yield: 483 mg, 99%; LCMS m/z 536.8 [M+1]⁺; ¹H NMR (300 MHz, Chloroform-d) δ 6.33 (t, J=5.8 Hz, 3H), 3.48 (s, 2H), 3.36-3.17 (m, 12H), 2.33-2.12 (m, 6H), 1.74-1.51 (m, 18H).

To a stirred solution of dodecanedioic acid (44G) (1.00 eq, 610 mg, 2.65 mmol) in THF (10 mL) under nitrogen was added sequentially: a solution of pentafluorophenol (2.50 eq, 1.22 g, 6.62 mmol) in THF (1 mL), EDC.HCl (2.20 eq, 1.12 g, 5.83 mmol), and then DIPEA (2.50 eq, 1.2 mL, 6.62 mmol). The resulting white mixture was stirred at room temperature under nitrogen for 4 h. The reaction mixture was partitioned between ethyl acetate and 1 N HCl in water. The organics were washed twice with brine, dried over magnesium sulfate, filtered, concentrated on a rotary evaporator, and purified via silica gel chromatography (0-50% ethyl acetate in hexanes) to afford Compound 44H as a white solid. Yield: 1.04 g, 70%; ¹H NMR (300 MHz, Chloroform-d) δ 2.66 (t, J=7.4 Hz, 4H), 1.77 (p, J=7.2 Hz, 4H), 1.48-1.22 (m, 12H).

Compound 44F (1.00 eq, 66.3 mg, 0.111 mmol), Compound 44H (3.00 eq, 188 mg, 0.334 mmol), DIPEA (5.00 eq, 0.097 mL, 0.557 mmol), and 1,4-dioxane (0.2500 mL) were combined in a sealable vessel with a stirbar, sealed, stirred, and heated at 80° C. with a heating block for 30 min. After cooling to room temperature volatiles were removed on a rotary evaporator at 30° C. The residue was taken up in a mixture of NMP, ethanol, and acetic acid, filtered, and purified via preparatory HPLC (30-90% acetonitrile in water with 0.1% TFA). Fractions containing the desired product were combined and lyophilized to dryness to afford Compound 44I as a yellow waxy solid. Yield: 27.8 mg, 27%; LCMS m/z 914.7 [M+1]⁺; ¹H NMR (300 MHz, Chloroform-d) δ 7.53 (s, 1H), 6.46 (t, J=6.1 Hz, 3H), 3.38-3.13 (m, 12H), 2.66 (t, J=7.5 Hz, 2H), 2.35-2.11 (m, 8H), 2.09-1.94 (m, 6H), 1.84-1.69 (m, 2H), 1.66-1.51 (m, 14H), 1.44-1.22 (m, 12H).

Compound 40A (3.20 eq, 52.5 mg, 0.107 mmol), Compound 44I (1.00 eq, 30.7 mg, 0.0336 mmol), and NMP (0.6 mL) were combined in a 1 dram vial with a stirbar, capped and stirred at room temperature. After 5 min, [(CH₃CN)₄Cu]PF₆ (7.00 eq, 87.6 mg, 0.235 mmol) was added. The resulting light yellow solution was capped and stirred at room temperature for 1 h. The reaction mixture slowly turned more green-colored. The reaction mixture was diluted with a mixture of NMP and acetic acid, filtered, and purified via preparatory HPLC (20-60% acetonitrile in water with 0.1% TFA). Fractions containing the desired product were combined and lyophilized to dryness to afford Compound I-44 as a white solid. Yield: 29.1 mg, 36%; ¹H NMR (300 MHz, DMSO-d₆ with D₂O) δ 7.84 (s, 3H), 7.13 (d, J=8.5 Hz, 6H), 7.00 (d, J=8.4 Hz, 6H), 5.34 (s, 3H), 4.27 (bs, 6H), 3.72-2.37 (m, 42H), 2.10-1.00 (m, 56H)

### Example 45: Synthesis of Compound I-45

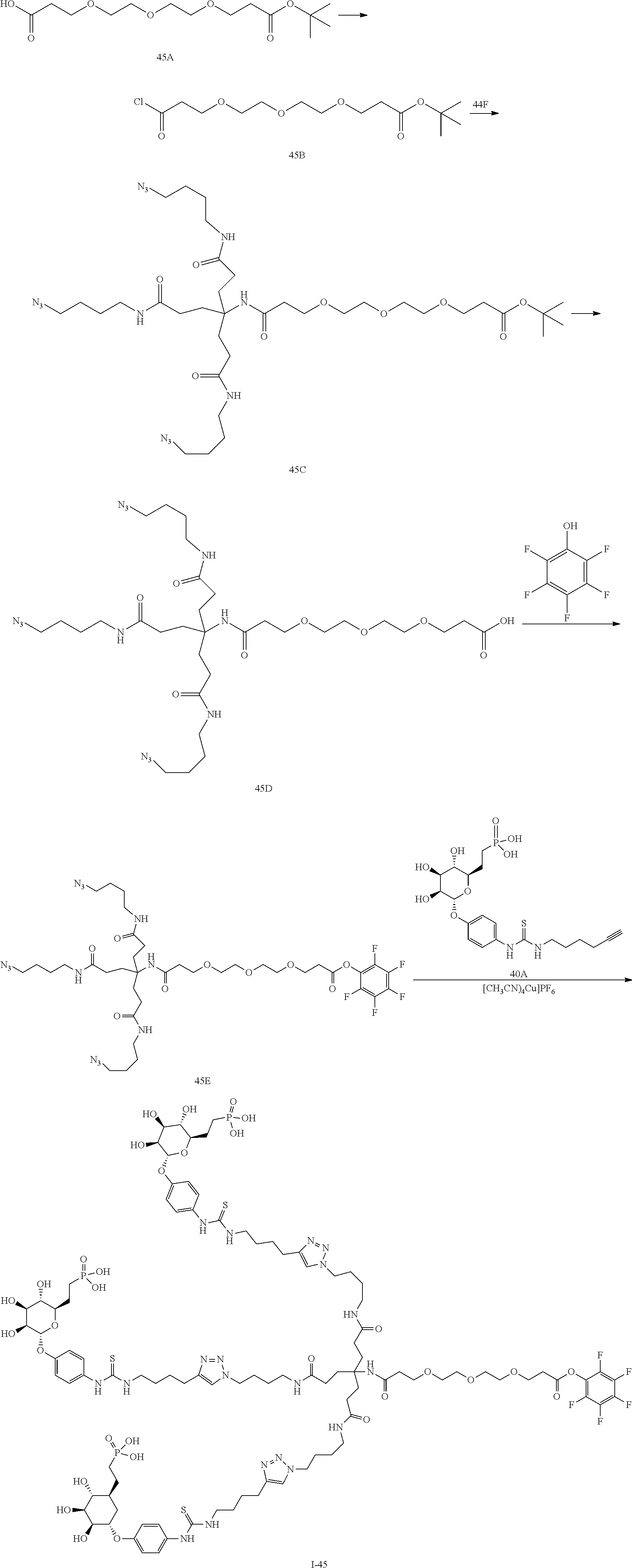

To a stirred solution of 2,2-dimethyl-4-oxo-3,7,10,13-tetraoxahexadecan-16-oic acid (45A) (1.00 eq, 102 mg, 0.333 mmol) in DCM (1 mL) at room temperature under nitrogen was added oxalyl chloride (2 M in methylene chloride) (1.15 eq, 0.19 mL, 0.383 mmol) and then DMF (1 microliter). The resulting clear solution was stirred at room temperature under nitrogen for 40 min. Vigorous bubbling was observed. Volatiles were blown off with a fast stream of nitrogen. The residue was dried under high vacuum to afford Compound 45B as a yellow oil which was used in the next step without purification.

A solution of Compound 44F (1.00 eq, 62.0 mg, 0.104 mmol) and DIPEA (6.00 eq, 0.11 mL, 0.625 mmol) in DCM (0.2000 mL) was added to Compound 45B (3.00 eq, 102 mg, 0.313 mmol) in a 1 dram vial with a stirbar. The resulting yellow solution was capped and stirred at room temperature for 30 min. Volatiles were blown off with a fast stream of nitrogen. The residue was taken up in a mixture of NMP, ethanol, and acetic acid, filtered, and purified via preparatory HPLC (20-100% acetonitrile in water with 0.1% TFA). Fractions containing the desired product were combined and concentrated considerably at 30° C. on a rotary evaporator, the remainder was lyophilized to dryness to afford Compound 45C as a colorless oil. Yield: 72.3 mg, 84%; LCMS m/z 824.7 [M+1]⁺; ¹H NMR (300 MHz, Chloroform-d) δ 7.03 (bs, 1H), 6.74 (bs, 2H), 5.96 (bs, 1H), 3.76-3.52 (m, 12H), 3.33-3.13 (m, 12H), 2.48 (t, J=6.4 Hz, 2H), 2.38 (t, J=5.6 Hz, 2H), 2.29-2.11 (m, 6H), 2.08-1.87 (m, 6H), 1.68-1.46 (m, 12H), 1.43 (s, 9H).

TFA (3 mL) was added to Compound 45C 1.00 eq, 70.9 mg, 0.0861 mmol) in a round bottom flask with a stirbar. The resulting solution was capped and stirred at room temperature for 3 h and then all volatiles were removed on a rotary evaporator. The residue was taken up in a mixture of NMP, ethanol, and acetic acid, filtered, and purified via preparatory HPLC (15-80% acetonitrile in water with 0.1% TFA). Fractions containing the desired product were combined and lyophilized to dryness to afford Compound 45D as a colorless oil. Yield: 42.5 mg, 64%; LCMS m/z 768.7 [M+1]⁺; ¹H NMR (300 MHz, Chloroform-d) δ 7.18 (t, J=6.0 Hz, 3H), 7.08 (bs, 1H), 3.84-3.49 (m, 12H), 3.39-3.11 (m, 12H), 2.60 (t, J=5.5 Hz, 2H), 2.43 (t, J=5.6 Hz, 2H), 2.33-2.16 (m, 6H), 2.10-1.90 (m, 6H), 1.69-1.45 (m, 12H).

Compound 45D (1.00 eq, 37.4 mg, 0.0487 mmol), DCC (1.80 eq, 18.1 mg, 0.0877 mmol), pentafluorophenol (2.50 eq, 22.4 mg, 0.122 mmol), DMAP (0.0200 eq, 0.12 mg, 0.000974 mmol), and DMF (0.3000 mL) were combined in a 1 dram vial with a stirbar, capped, and stirred at room temperature for 3 h. More DCC (10 mg, 0.048 mmol) was added. The resulting mixture was capped and stirred at room temperature for 2.5 h. The reaction mixture was diluted with a mixture of NMP, ethanol, and acetic acid, filtered, and purified via preparatory HPLC (20-90% acetonitrile in water with 0.1% TFA). Fractions containing the desired product were combined and lyophilized to dryness to afford Compound 45E as a colorless oil. Yield: 36.1 mg, 79%; LCMS m/z 934.7 [M+1]+; ¹H NMR (300 MHz, Chloroform-d) δ 7.03 (bs, 1H), 6.73 (t, J=6.0 Hz, 3H), 3.85 (t, J=6.1 Hz, 2H), 3.72 (t, J=5.5 Hz, 2H), 3.67-3.57 (m, 8H), 3.34-3.16 (m, 12H), 2.93 (t, J=6.1 Hz, 2H), 2.39 (t, J=5.6 Hz, 2H), 2.29-2.15 (m, 6H), 2.05-1.90 (m, 6H), 1.67-1.47 (m, 12H).

Compound 40A (3.00 eq, 56.6 mg, 0.116 mmol), Compound 45E (1.00 eq, 36.1 mg, 0.0387 mmol), and NMP (0.6000 mL) were combined in a 1 dram vial with a stirbar, capped and stirred at room temperature. After 5 min [(CH₃CN)₄Cu]PF₆ (7.00 eq, 101 mg, 0.271 mmol) was added. The resulting light yellow solution was capped and stirred at room temperature for 30 min. The reaction mixture slowly turned more green-colored. The reaction mixture was diluted with a mixture of NMP and acetic acid, filtered, and purified via preparatory HPLC (20-55% acetonitrile in water with 0.1% TFA). Fractions containing the desired product were combined and lyophilized to dryness to afford Compound I-45 as a white solid. Yield: 54.1 mg, 58%; ¹H NMR (300 MHz, DMSO-d₆ with D₂O) δ 7.83 (s, 3H), 7.13 (d, J=8.5 Hz, 6H), 7.00 (d, J=8.6 Hz, 6H), 5.34 (s, 3H), 4.26 (bs, 6H), 3.88-2.87 (m, 40H), 2.64-2.53 (m, 6H), 2.04-1.40 (m, 40H), 1.36-1.11 (m, 8H).

### Example 46: Synthesis of Compound I-46

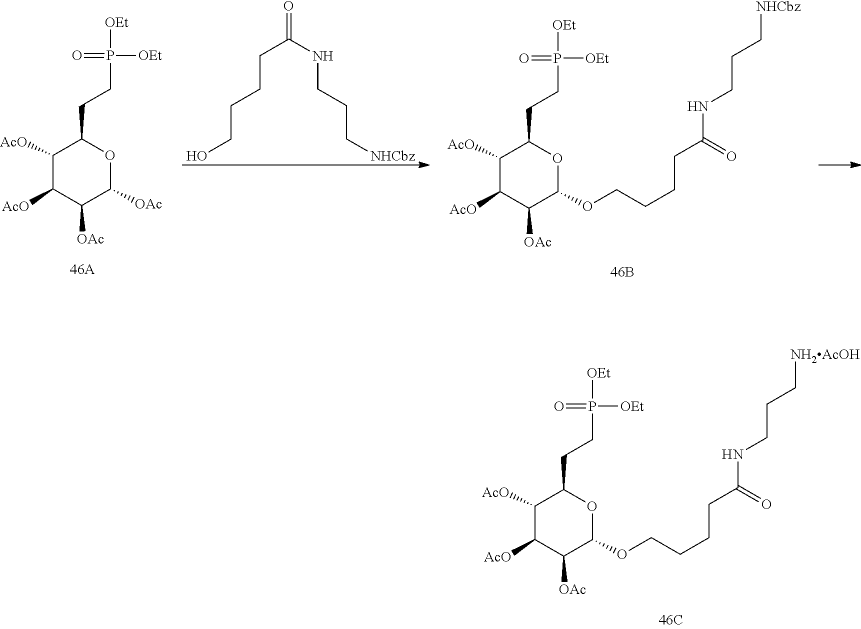

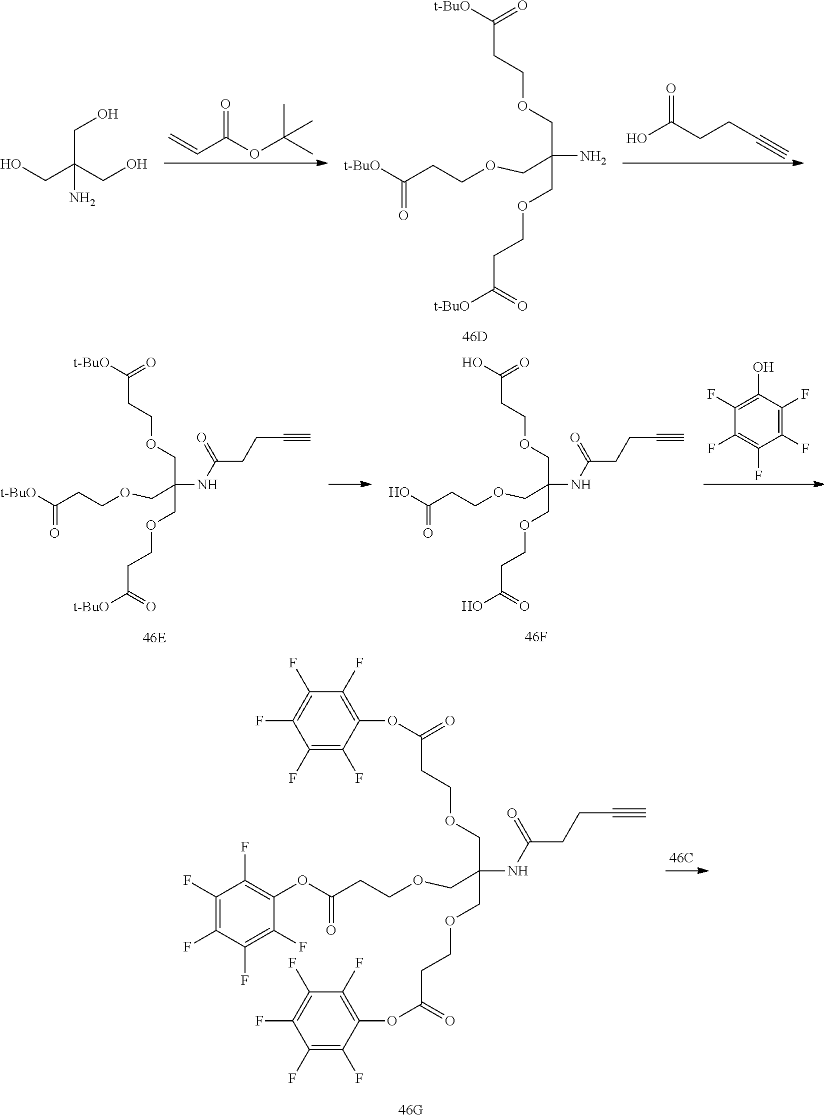

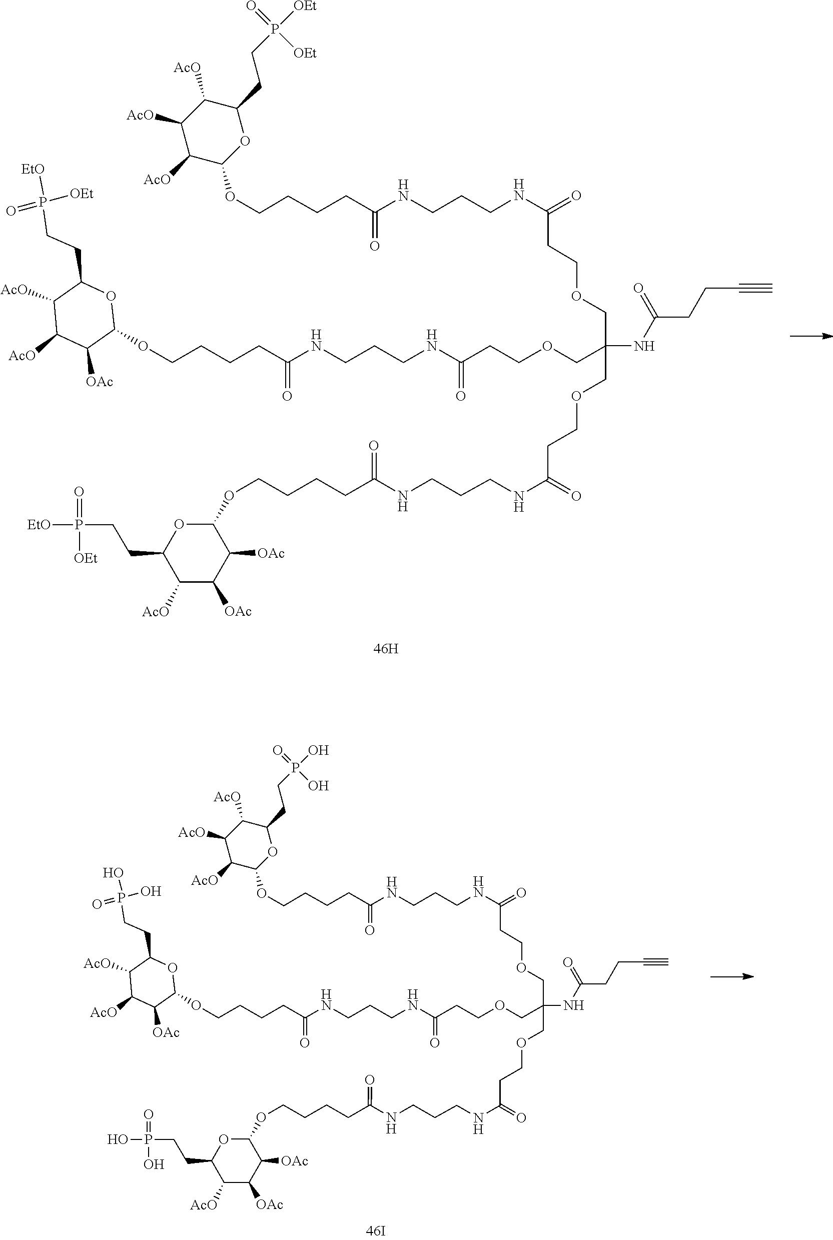

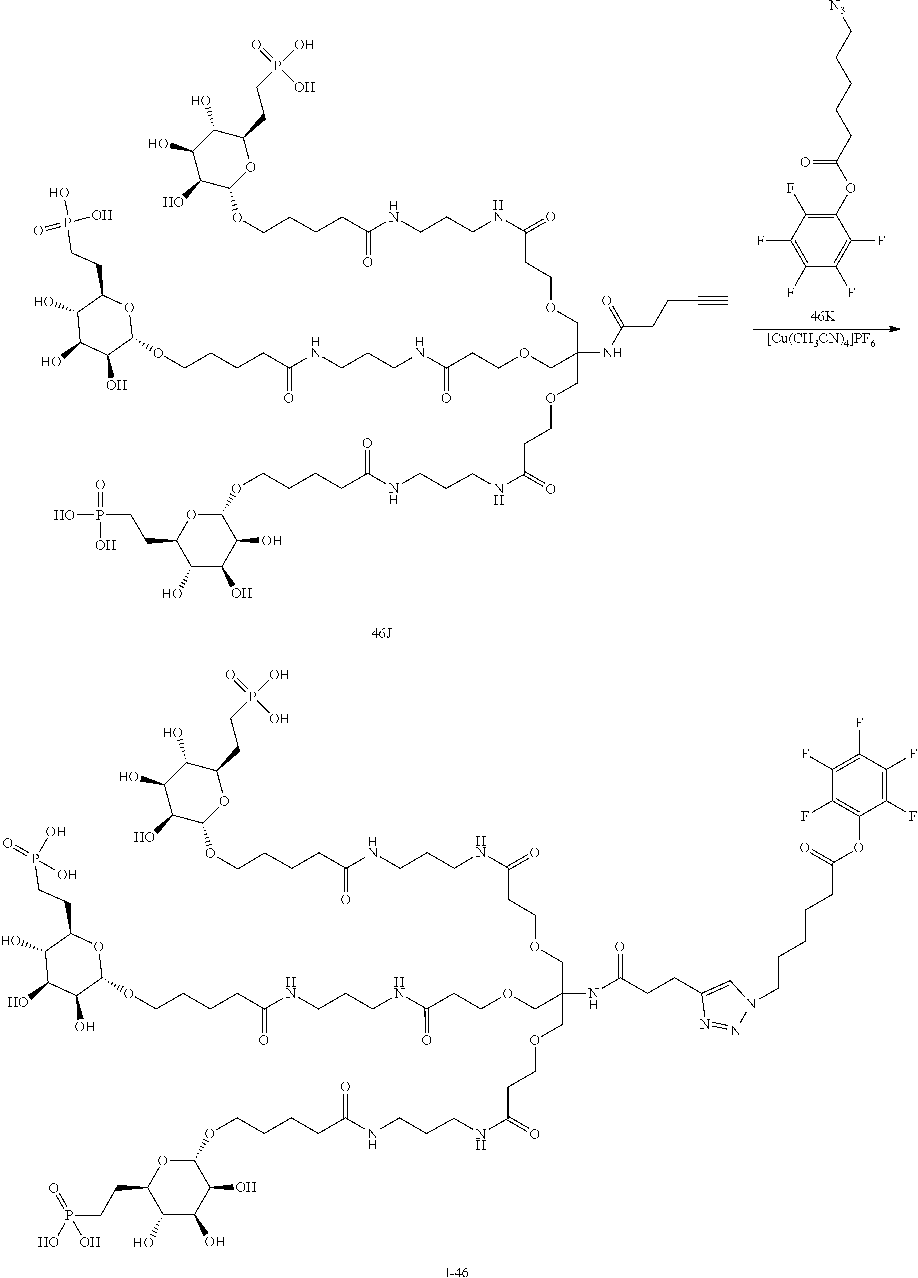

A solution of (2R,3S,4S,5R,6R)-6-(2-(diethoxyphosphoryl)ethyl)tetrahydro-2H-pyran-2,3,4,5-tetrayl tetraacetate (46A) (1.0 eq, 5.00 g, 10.4 mmol) and benzyl (3-(5-hydroxypentanamido)propyl)carbamate (2.0 eq, 6.39 g, 20.7 mmol) in DCM (100 mL) was cooled at 0° C., BF₃.Et₂O (12.0 eq, 15.4 mL, 124.0 mmol) was added dropwise and reaction mixture was heated at 50° C. for 16 h. Reaction was monitored by LCMS. After completion, reaction mixture was cooled at 0° C. and neutralized with triethylamine. Then, reaction mixture was diluted with DCM and washed with water. Organic layer was dried over anhydrous sodium sulfate, filtered and concentrated to get crude which was purified by reverse column chromatography using C-18 column and 20-50% acetonitrile in water to afford Compound 46B as a colorless viscous liquid. Yield: 3.10 g, 35.83%; LCMS m/z 731.29 [M+1]⁺.

To a solution of Compound 46B (1.0 eq, 2.6 g, 3.56 mmol) in methanol (26 mL), acetic acid (2.6 mL) and Palladium on carbon (10%) (1.3 g) was added and reaction mixture was stirred under hydrogen gas atmosphere at room temperature for 3 h. After completion, reaction mixture was filtered, filtrate was concentrated and dried to afford Compound 46C as colorless viscous liquid. Yield: 3.1 g (Crude); LCMS m/z 597.27 [M+1]⁺

A solution of 2-amino-2-(hydroxymethyl)propane-1,3-diol (1, 1.0 eq, 19.0 g, 157.0 mmol) in DMSO (76 mL) was cooled at 0° C., NaOH solution (5M) (4.0 mL) and tert-butyl acrylate (10.0 eq, 251.0 mL, 1570.0 mmol) was added dropwise and reaction mixture was stirred at room temperature for 16 h. Reaction was monitored by ELSD. After completion, water was added to reaction mixture and extracted with ethyl acetate. The organic layer was dried over anhydrous sodium sulfate, filtered and concentrated to afford Compound 46D as a colorless viscous liquid. Yield: 76.0 g, 95.83%; LCMS m/z 506.33 [M+1]⁺.

To a solution of Compound 46D (1.0 eq, 20.0 g, 39.6 mmol) and pent-4-ynoic acid (1.1 eq, 4.27 g, 43.5 mmol) in DMF (200 mL), EDC.HCl (1.5 eq, 11.4 g, 59.3 mmol), 1-hydroxybenzotriazole (1.5 eq, 9.03 g, 59.3 mmol) and NMP (2.0 eq, 7.84 mL, 79.1 mmol) were added and reaction mixture was stirred at room temperature for 16 h. After completion, water was added to reaction mixture and extracted with ethyl acetate. The organic layer was washed with water, dried over anhydrous sodium sulfate, filtered and concentrated to get crude which was purified by column chromatography on silica gel to afford Compound 46E as a colorless viscous liquid. Yield: 12.0 g, 44.87%; LCMS m/z 586.35 [M+1]⁺.

A solution of Compound 46E (1.0 eq, 10.5 g, 17.9 mmol) in formic acid (105 mL) was stirred at room temperature for 16 h. After completion, reaction mixture was concentrated and dried to afford Compound 46F as a colorless viscous liquid. Yield: 9.7 g (Crude); LCMS m/z 418.16 [M+1]⁺.

A solution of Compound 46F (1.0 eq, 2.6 g, 6.2 mmol) in ethyl acetate (26 mL) was cooled at 0° C., pentafluorophenol (3.0 eq, 3.4 g, 18.6 mmol) and DIC (4.0 eq, 3.8 mL, 24.8 mmol) were added and reaction mixture was stirred at room temperature for 16 h. After completion, reaction mixture was filtered through celite bed and celite bed was washed with ethyl acetate. The filtrate was concentrated to get crude which was purified by column chromatography on silica gel to afford Compound 46G as a colorless viscous liquid. Yield: 3.6 g, 63.2%; LCMS m/z 916.12 [M+1]⁺.

A solution of Compound 46F (1.0 eq, 1.1 g, 1.2 mmol) and Compound 46C (4.0 eq, 3.1 g, 4.81 mmol) in DMF (22 mL) was stirred at room temperature for 1 h. Reaction was monitored by LCMS. After completion, reaction mixture was concentrated, washed with diethyl ether (3-4 times) and dried to afford Compound 46H as a light brown viscous liquid. Yield: 2.5 g (Crude); LCMS m/z 1076.95 [(M/2)+1]⁺

A solution of Compound 46H (1.0 eq, 2.5 g, 1.16 mmol) in DCM (25 mL) was cooled at 0° C., pyridine (30.0 eq, 3.0 mL, 34.8 mmol) and TMSBr (30.0 eq, 4.6 mL, 34.8 mmol) were added and reaction mixture was stirred at room temperature for 3 h. After completion, reaction mixture was quenched with water and concentrated to get crude. Crude was diluted with acetonitrile and purified by prep HPLC (20-42% acetonitrile in water with 5 mM ammonium acetate). Fractions containing the desired product were combined and lyophilized to dryness to afford Compound 461 as a light brown sticky solid. Yield: 0.500 g; 21.73%; LCMS m/z 993.4 [(M/2)+1]⁺.

To a solution of Compound 461 (1.0 eq, 0.620 g, 0.31 mmol) in methanol (6 mL), sodium methoxide (25% solution in methanol) (10.0 eq, 0.76 mL, 3.1 mmol) was added and reaction mixture was stirred at room temperature for 1 h. After completion, reaction mixture was neutralized with Dowex 50WX8 hydrogen form (200-400 mesh) and filtered through syringe filter. The filtrate was concentrated and dried to afford Compound 46J as a light orange solid. Yield: 0.500 g; 83.66%; LCMS m/z 803.8 [(M/2)+1]⁺

To a solution of Compound 46J (1.0 eq, 0.025 g, 0.015 mmol) in DMSO (0.5 mL), 2,3,4,5,6-pentafluorophenyl 6-azidohexanoate (1.2 eq, 0.006 g, 0.018 mmol) was added and stirred for 5 minutes. Then, [(CH₃CN)₄Cu]PF₆ (2.8 eq., 0.016 g, 0.043 mmol) was added and reaction mixture was stirred at room temperature for 1 h. After completion, reaction mixture was diluted with acetonitrile and purified by prep HPLC (25-55% acetonitrile in water with 0.1% TFA). Fractions containing the desired product were combined and lyophilized to dryness to afford Compound I-46 as an off white solid. Yield: 0.0035 g, 11.6%; LCMS m/z 965.68 [(M/2)+1]⁺; ¹H NMR (400 MHz, DMSO-d₆) δ 7.87 (t, J=4.8 Hz, 3H), 7.82-7.78 (m, 4H), 7.20-7.17 (m, 1H), 4.54 (s, 3H), 4.30 (t, J=7.2 Hz, 3H), 3.70 (bs, 1H), 3.56-3.52 (m, 6H), 3.39-3.25 (m, 25H), 3.22 (d, J=6.4 Hz, 8H), 3.02 (bs, 13H), 2.78 (t, J=7.6 Hz, 3H), 2.41 (t, J=8.0 Hz, 2H), 2.27 (t, J=6.0 Hz, 6H), 2.10-2.06 (m, 6H), 2.05-1.98 (m, 3H), 1.84-1.77 (m, 3H), 1.74-1.64 (m, 6H), 1.60-1.39 (m, 25H), 1.35-1.28 (m, 3H).

### Example 47: Synthesis of Compound I-47

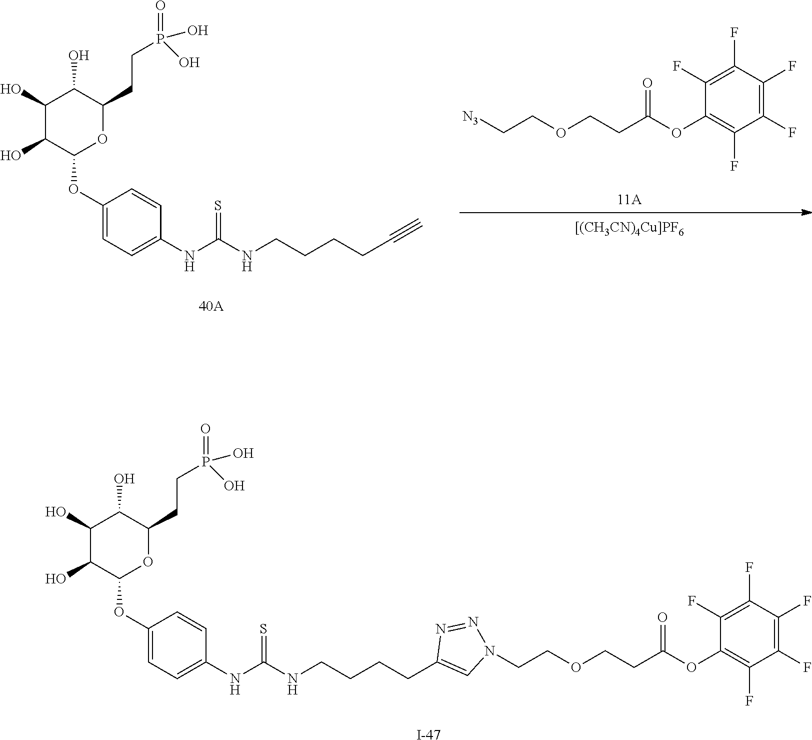

To Compound 40A (1.00 eq, 28.6 mg, 0.0585 mmol) in a 1 dram vial with a stirbar was added a solution of Compound 11A (1.20 eq, 22.8 mg, 0.0703 mmol) in NMP (0.6 mL) followed by [(CH₃CN)₄Cu]PF₆ (2.50 eq, 54.6 mg, 0.146 mmol). The resulting clear yellow solution was capped and stirred at room temperature for 20 min. The reaction mixture was diluted with mixture of NMP, ethanol, and acetic acid, filtered, and purified via preparatory HPLC (15-50% acetonitrile in water with 0.1% TFA). Fractions containing the desired product were combined and lyophilized to dryness to afford Compound I-47 as a white solid. Yield: 35.8 mg, 75%; LCMS m/z 814.4 [M+1]⁺; ¹H NMR (300 MHz, DMSO-d₆ with D₂O) δ 7.73 (s, 1H), 7.31-7.15 (m, 2H), 7.04-6.85 (m, 2H), 5.31 (s, 1H), 4.45 (t, J=5.2 Hz, 2H), 3.89-3.23 (m, 8H), 3.03-2.91 (m, 2H), 2.62-2.39 (m, 4H), 2.00-1.81 (m, 1H), 1.71-1.39 (m, 6H), 1.32-1.09 (m, 1H).

### Example 48: Synthesis of Compound I-48

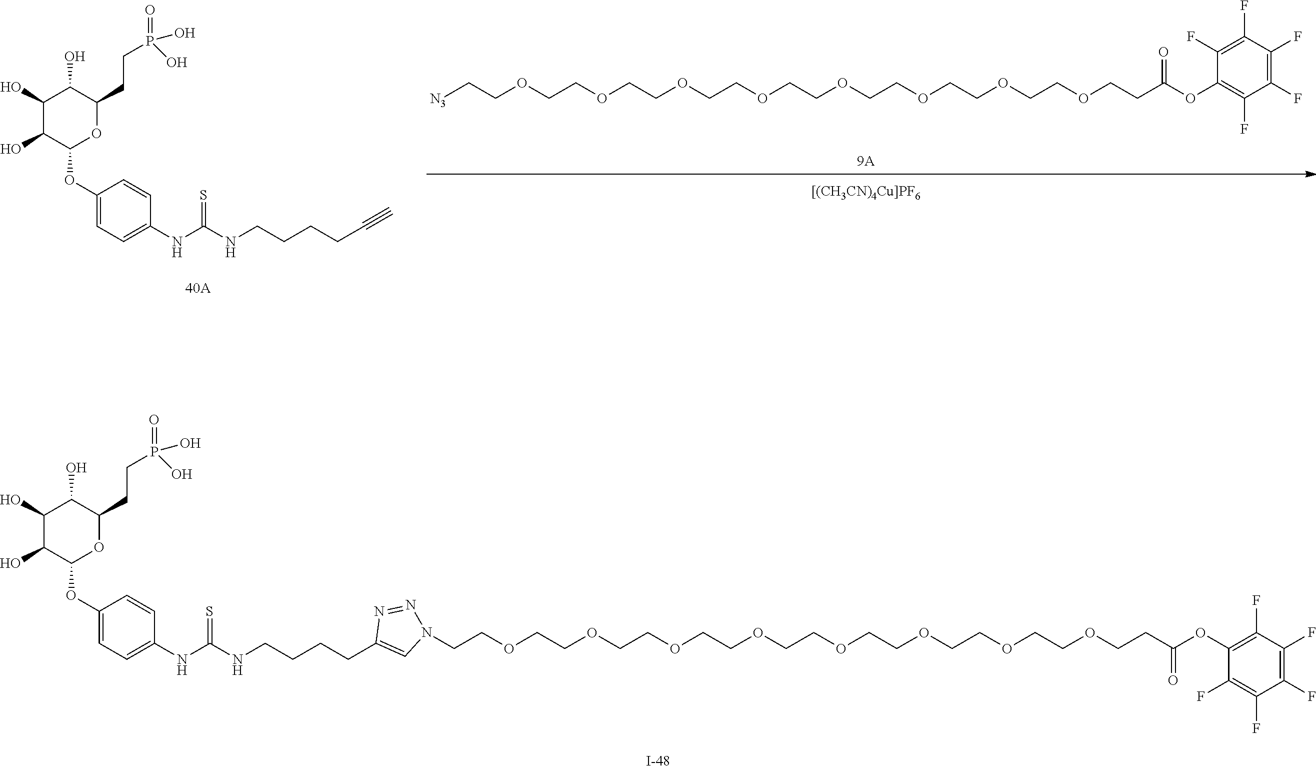

To Compound 40A (1.00 eq, 39.9 mg, 0.0817 mmol) in a 1 dram vial with a stirbar was added a solution of Compound 9A (1.20 eq, 62.1 mg, 0.0980 mmol) in NMP (0.6 mL) followed by [(CH₃CN)₄Cu]PF₆ (2.50 eq, 76.1 mg, 0.204 mmol). The resulting clear yellow solution was capped and stirred at room temperature for 20 min. The reaction mixture was diluted with mixture of NMP, ethanol, and acetic acid, filtered, and purified via preparatory HPLC (15-50% acetonitrile in water with 0.1% TFA). Fractions containing the desired product were combined and lyophilized to dryness to afford Compound I-48 as a white solid. Yield: 64.4 mg, 70%; LCMS m/z 1122.6 [M+1]⁺; ¹H NMR (300 MHz, DMSO-d₆ with D₂O) δ 7.80 (s, 1H), 7.25 (d, J=8.6 Hz, 2H), 6.97 (d, J=8.5 Hz, 2H), 5.32 (s, 1H), 4.44 (t, J=5.2 Hz, 2H), 3.85-3.20 (m, 36H), 2.99 (t, J=5.8 Hz, 2H), 2.67-2.37 (m, 4H), 2.02-1.82 (m, 1H), 1.70-1.38 (m, 6H), 1.30-1.05 (m, 1H).

### Example 49: Synthesis of Compound I-49

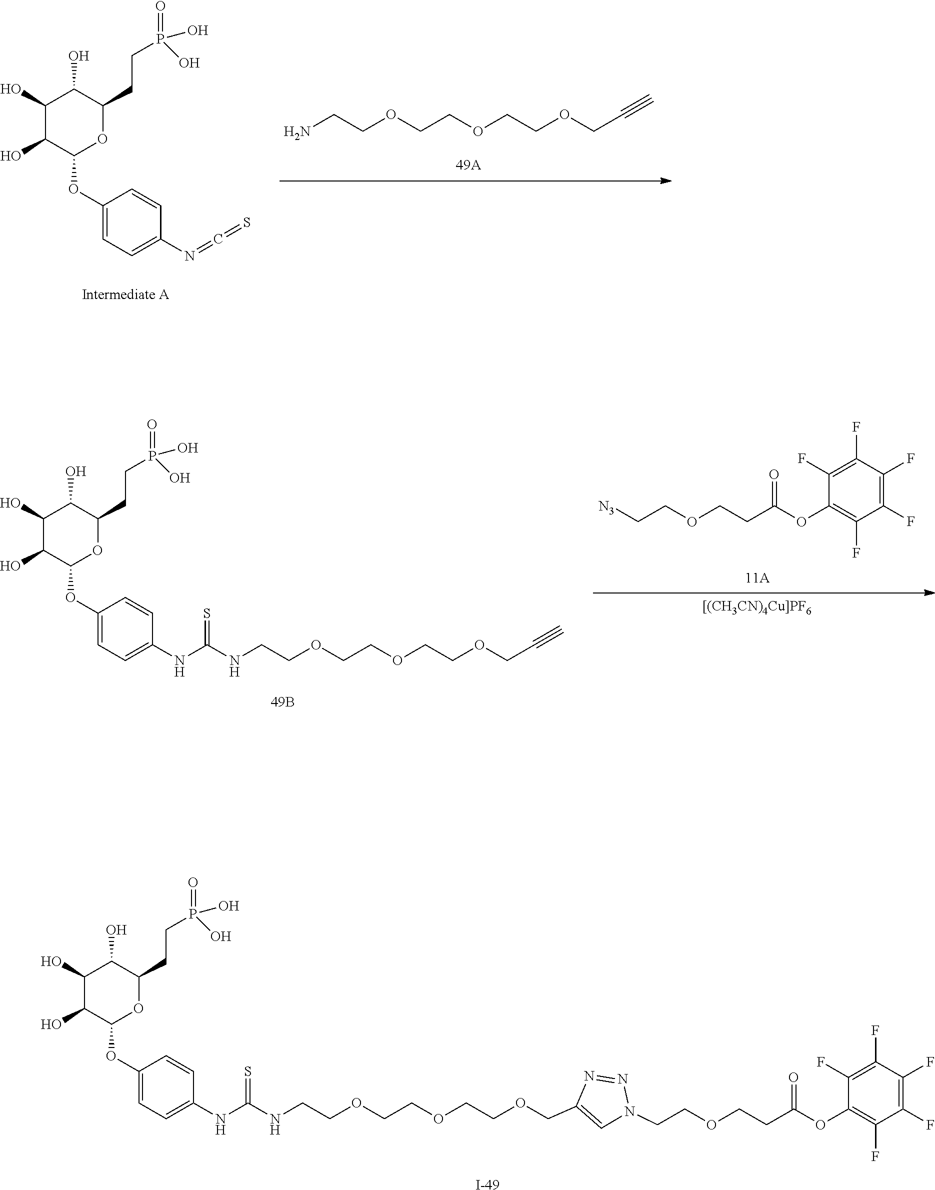

A solution of 2-(2-(2-(prop-2-yn-1-yloxy)ethoxy)ethoxy)ethan-1-amine (49A) (1.40 eq, 30.7 mg, 0.164 mmol) in NMP (0.6 mL) was added to Intermediate A (1.00 eq, 45.8 mg, 0.117 mmol) in a 1 dram vial with a stirbar. The resulting mixture was capped and stirred at room temperature for 18 h. Solids slowly dissolved to give a clear yellow solution. The reaction mixture was diluted with mixture of ethanol and acetic acid, filtered, and purified via preparatory HPLC (10-30% acetonitrile in water with 0.1% TFA). Fractions containing the desired product were combined. Most of the solvent was removed on a rotary evaporator at 29° C. and the remainder was lyophilized to dryness to afford Compound 49B as a white solid. Yield: 47.7 mg, 70%; LCMS m/z 579.4 [M+1]⁺; ¹H NMR (300 MHz, DMSO-d₆ with D₂O) δ 7.28 (d, J=8.6 Hz, 2H), 6.99 (d, J=8.5 Hz, 2H), 5.32 (s, 1H), 4.16-4.05 (m, 2H), 3.85-3.76 (m, 1H), 3.74-3.41 (m, 13H), 3.40-3.24 (m, 3H), 2.02-1.82 (m, 1H), 1.72-1.40 (m, 2H), 1.34-1.07 (m, 1H)

To Compound 49B (1.00 eq, 43.2 mg, 0.0747 mmol) in a 1 dram vial with a stirbar was added a solution of Compound 11A (1.20 eq, 29.1 mg, 0.0896 mmol) in NMP (0.6 mL) followed by [(CH₃CN)₄Cu]PF₆ (2.50 eq, 69.6 mg, 0.187 mmol). The resulting clear yellow solution was capped and stirred at room temperature for 20 min. The reaction mixture was diluted with mixture of NMP, ethanol, and acetic acid, filtered, and purified via preparatory HPLC (15-50% acetonitrile in water with 0.1% TFA). Fractions containing the desired product were combined and lyophilized to dryness to afford Compound I-49 as a white solid. Yield: 44.2 mg, 66%; LCMS m/z 904.4 [M+1]⁺; ¹H NMR (300 MHz, DMSO-d₆ with D₂O) δ 7.96 (s, 1H), 7.27 (d, J=8.5 Hz, 2H), 6.98 (d, J=8.7 Hz, 2H), 5.32 (s, 1H), 4.57-4.40 (m, 4H), 3.89-3.22 (m, 18H), 2.98 (t, J=5.8 Hz, 2H), 2.59-2.35 (m, 2H), 2.02-1.82 (m, 1H), 1.73-1.41 (m, 2H), 1.34-1.11 (m, 1H).

### Example 50: Synthesis of Compound I-50

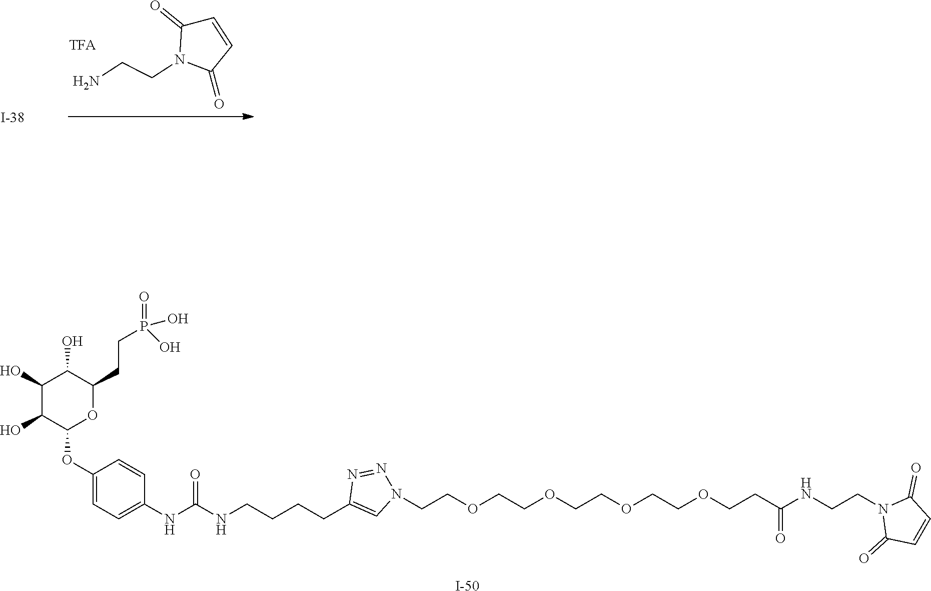

To Compound I-38 (1.00 eq, 37.4 mg, 0.0402 mmol) in a vial with a stirbar was added a solution of 1-(2-aminoethyl)-1H-pyrrole-2,5-dione TFA salt (1.15 eq, 11.8 mg, 0.0463 mmol) and DIPEA (3.00 eq, 0.021 mL, 0.121 mmol) in NMP (0.5 mL). The resulting clear slightly yellow solution was capped and stirred at room temperature for 20 min. The reaction mixture was diluted with acetic acid, filtered, and purified via preparatory HPLC (10-35% acetonitrile in water with 0.1% TFA). Fractions containing the desired product were combined and lyophilized to dryness to afford Compound I-50 as a white solid. Yield: 25.8 mg, 72%; LCMS m/z 886.6 [M+1]⁺; ¹H NMR (300 MHz, DMSO-d₆ with D₂O) δ 7.78 (s, 1H), 7.29-7.18 (m, 2H), 6.95-6.81 (m, 4H), 5.24 (s, 1H), 4.42 (t, J=5.1 Hz, 2H), 3.97-3.25 (m, 22H), 3.21-3.11 (m, 2H), 3.05 (t, J=6.6 Hz, 2H), 2.66-2.54 (m, 2H), 2.25-2.14 (m, 2H), 2.03-1.81 (m, 1H), 1.69-1.34 (m, 6H), 1.31-1.07 (m, 1H).

### Example 51: Synthesis of Compound I-51

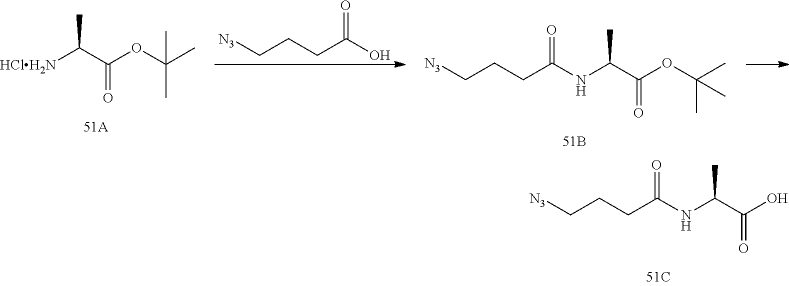

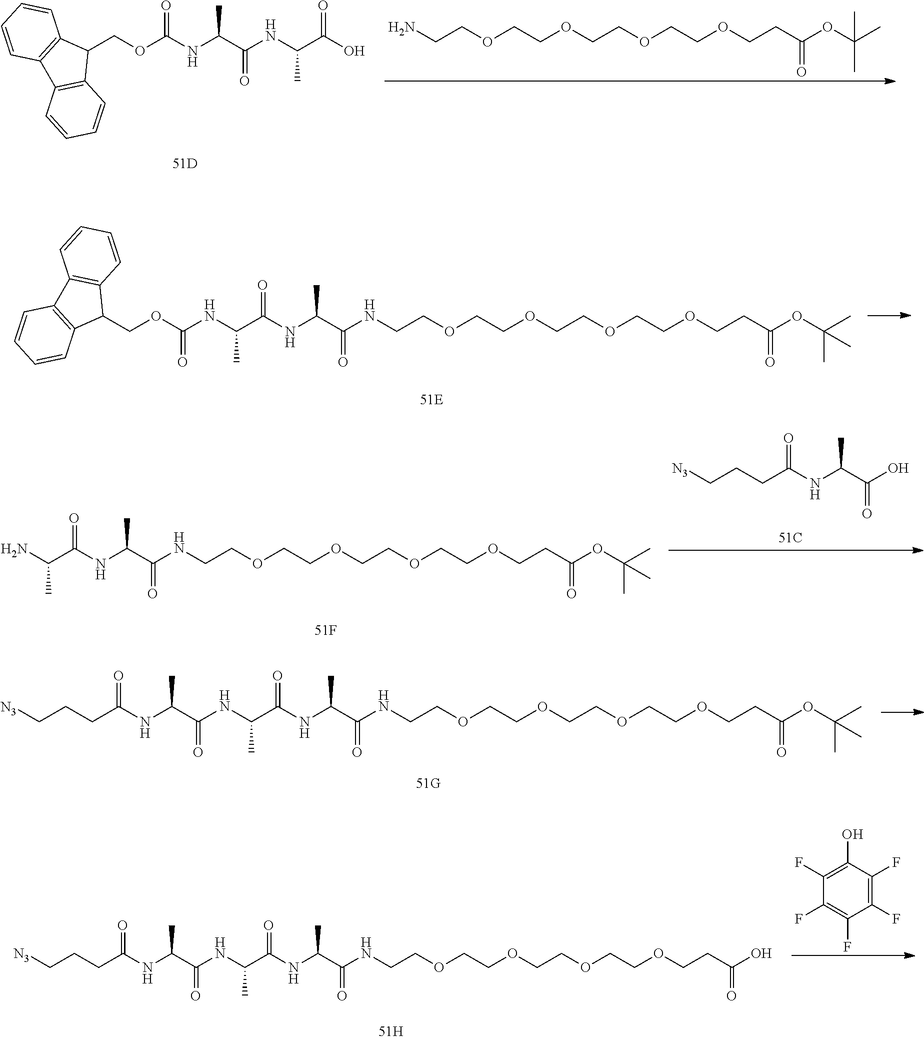

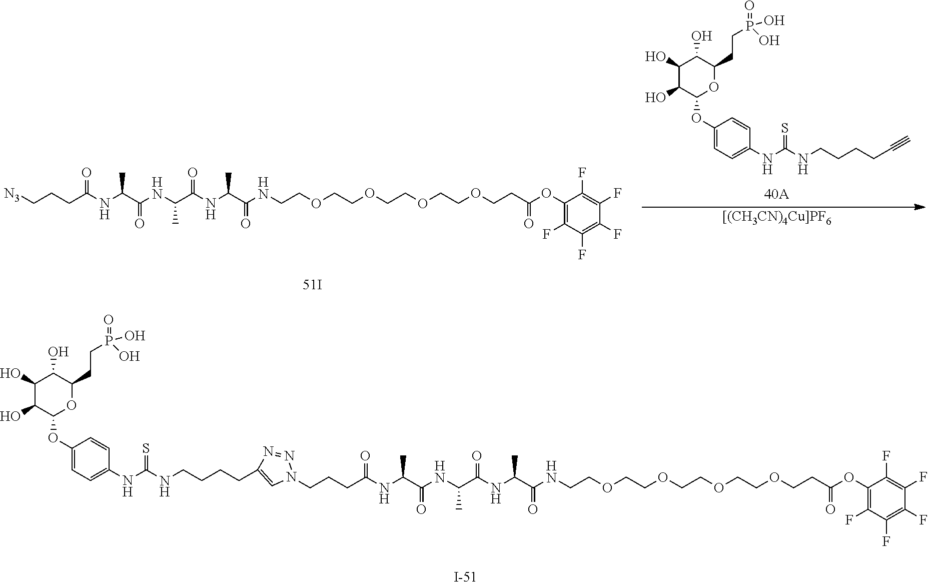

tert-butyl L-alaninate hydrochloride (51A) (2.80 g, 0.015 mol) and 4-azidobutanoic acid (2.0 g, 0.015 mol) in THF (30 mL) at 0° C., were added DIPEA (7.84 mL, 0.045 mol) and (Benzotriazol-1-yloxy) tripyrrolidinophosphonium hexafluorophosphate (8.80 g, 0.017 mol). Reaction mixture was stirred at room temperature for 4 h and concentrated under reduced pressure to remove tetrahydrofuran. Crude residue obtained was purified by silica gel flash column chromatography eluting product in 30 to 50% ethyl acetate in hexanes as eluents to afford Compound 51B as pale yellow sticky gum Yield: 2.90 g (73%); LCMS m/z 257.15 [M+1]⁺.

To a solution of Compound 51B (2.90 g, 0.011 mol) in DCM (20.0 mL) at 0° C. was added 4N HCl in 1,4-dioxane (10.0 mL) and reaction mixture stirred at room temperature for 16 h. Reaction mixture concentrated under reduced pressure and dried under high vaccum to afford Compound 51C as pale yellow sticky gum. Yield: 2.10 g (92.71%) LCMS m/z 201.15 [M+1]⁺.

To a solution of (((9H-fluoren-9-yl) methoxy) carbonyl)-L-alanyl-L-alanine (51D) (2.50 g, 6.54 mmol) and tert-butyl 1-amino-3,6,9,12-tetraoxapentadecan-15-oate (2.10 g, 6.54 mmol) in THF (30 mL) at 0° C. were added DIPEA (3.42 mL, 19.62 mmol) and (benzotriazol-1-yloxy) tripyrrolidinophosphonium hexafluorophosphate (4.08 g, 7.84 mmol). Reaction mixture stirred at room temperature for 6 h. After completion reaction mixture partitioned in between ethyl acetate and water. Aqueous layer re-extracted with ethyl acetate and combined ethyl acetate layer washed with water, brine solution dried over anhydrous sodium sulfate and concentrated under reduced pressure to get crude product. Crude product obtained was purified by combiflash column chromatography eluting product in 5 to 7% methanol in DCM as eluents. Desired fractions were concentrated under reduced pressure to afford Compound 51E as pale yellow sticky gum. Yield: 3.60 g (80%); LCMS m/z 686.35 [M+1]⁺

To a solution of Compound 51E (3.60 g, 5.25 mmol) in N,N-DMF (15.00 mL) was added piperidine (5 mL) and reaction mixture stirred at room temperature for 1 h. TLC showed consumption of starting material. Reaction mixture concentrated under reduced pressure to obtain pale yellow sticky gum. Pale yellow sticky gum obtained was triturated with diethyl ether, pentane and dried under high vacuum to afford Compound 51F as pale yellow sticky gum. Yield:2.10 g (86.00%); ELSD-MS m/z 464.3 [M+1]⁺.

To a solution of Compound 51F (2.40 g, 5.18 mmol) in THF (30 mL) at 0° C. were added Compound 51C (1.55 g, 7.77 mmol), DIPEA (2.71 mL, 15.5 mmol) and 1-[bis(dimethylamino)methylene]-1H-1,2,3-triazolo[4,5-b]pyridinium 3-oxide hexafluorophosphate (2.33 g, 6.21 mmol). Reaction mixture was then stirred at room temperature for 3 h. Reaction mixture quenched by addition of water and extracted with ethyl acetate. Ethyl acetate layer dried over anhydrous sodium sulfate and concentrated under reduced pressure to get crude product. Crude product obtained was column purified by flash column chromatography eluting product in 8 to 10% methanol in DCM as eluents. Desired fractions were concentrated under reduced pressure to afford Compound 51G as off white solid. Yield: 1.47 g (44%); LCMS m/z 646.2 [M+1]⁺.

To a solution of Compound 51G (1.47 g, 2.28 mmol) in DCM (10 mL) at 0° C. was added 4M hydrochloric acid in 1,4-Dioxane (5.69 mL) and reaction mixture stirred at room temperature for 16 h. After completion reaction mixture was concentrated under reduced pressure, triturated with pentane and dried under high vacuum to afford Compound 51H as off white solid. Yield: 1.30 g (96.85%); LCMS m/z 590.30 [M+1]⁺

To a solution of Compound 51H (0.60 g, 1.02 mmol) in DMF ((20 mL) were added pentafluorophenol (0.281 g, 1.52 mmol) and DIC (0.24 mL, 1.52 mmol), Reaction mixture then stirred at room temperature for 6 h. ELSD-MS showed formation of desired product as well as presence of starting material, so again pentafluorophenol (0.281 g, 1.52 mmol) and DIPEA (0.24 mL, 1.52 mmol) were added to reaction mixture and reaction mixture stirred at room temperature for 16 h. Reaction mixture concentrated under reduced pressure to remove DMF and crude obtained was purified by preparatory HPLC (45-65% acetonitrile in water with 0.1% acetic acid). Fractions containing the desired product were combined and lyophilized to afford Compound 51I as off white solid. Yield: 0.368 g (47.86%); LCMS m/z 756.43 [M+1]⁺. ¹H NMR (400 MHz, DMSO-d₆) δ 8.09 (d, J=7.20 Hz, 1H), 8.00 (d, J=7.20 Hz, 1H), 7.84-7.80 (m, 2H), 4.24-4.16 (m, 3H), 3.76 (t, J=6.0 Hz, 2H), 3.55-3.49 (m, J=12H), 3.38 (t, J=6.0 Hz, 2H), 3.30 (s, 2H), 3.23-3.15 (m, 2H), 3.02 (t, J=5.60 Hz, 2H), 2.19 (t, J=7.20 Hz, 2H), 1.73 (quin, J=7.20 Hz, 2H), 1.21-1.16 (m, 9H).

To Compound 40A (1.05 eq, 15.3 mg, 0.0313 mmol), Compound 51I (1.00 eq, 22.5 mg, 0.0298 mmol), and NMP (0.35 mL) were combined in a 1 dram vial with a stirbar, capped and stirred at room temperature. After 5 min [(CH₃CN)₄Cu]PF₆ (2.50 eq, 27.7 mg, 0.0744 mmol) was added. The resulting light yellow solution was capped and stirred at room temperature for 20 min. The reaction mixture slowly became more green-colored. The reaction mixture was diluted with a mixture of NMP and acetic acid, filtered, and purified via preparatory HPLC (15-60% acetonitrile in water with 0.1% TFA). Fractions containing the desired product were combined and lyophilized to dryness to afford Compound I-51 as a white solid. Yield: 25.4 mg, 70%; LCMS m/z 1244.7 [M+1]+; ¹H NMR (300 MHz, DMSO-d₆ with D₂O) δ 7.82 (s, 1H), 7.24 (d, J=8.2 Hz, 2H), 6.98 (d, J=8.7 Hz, 2H), 5.32 (s, 1H), 4.28 (t, J=6.9 Hz, 2H), 4.22-4.07 (m, 3H), 3.88-2.89 (m, 26H), 2.67-2.38 (m, 2H), 2.15-2.04 (m, 2H), 2.04-1.83 (m, 3H), 1.67-1.41 (m, 6H), 1.32-1.03 (m, 10H).

### Example 52: Synthesis of Compound I-52

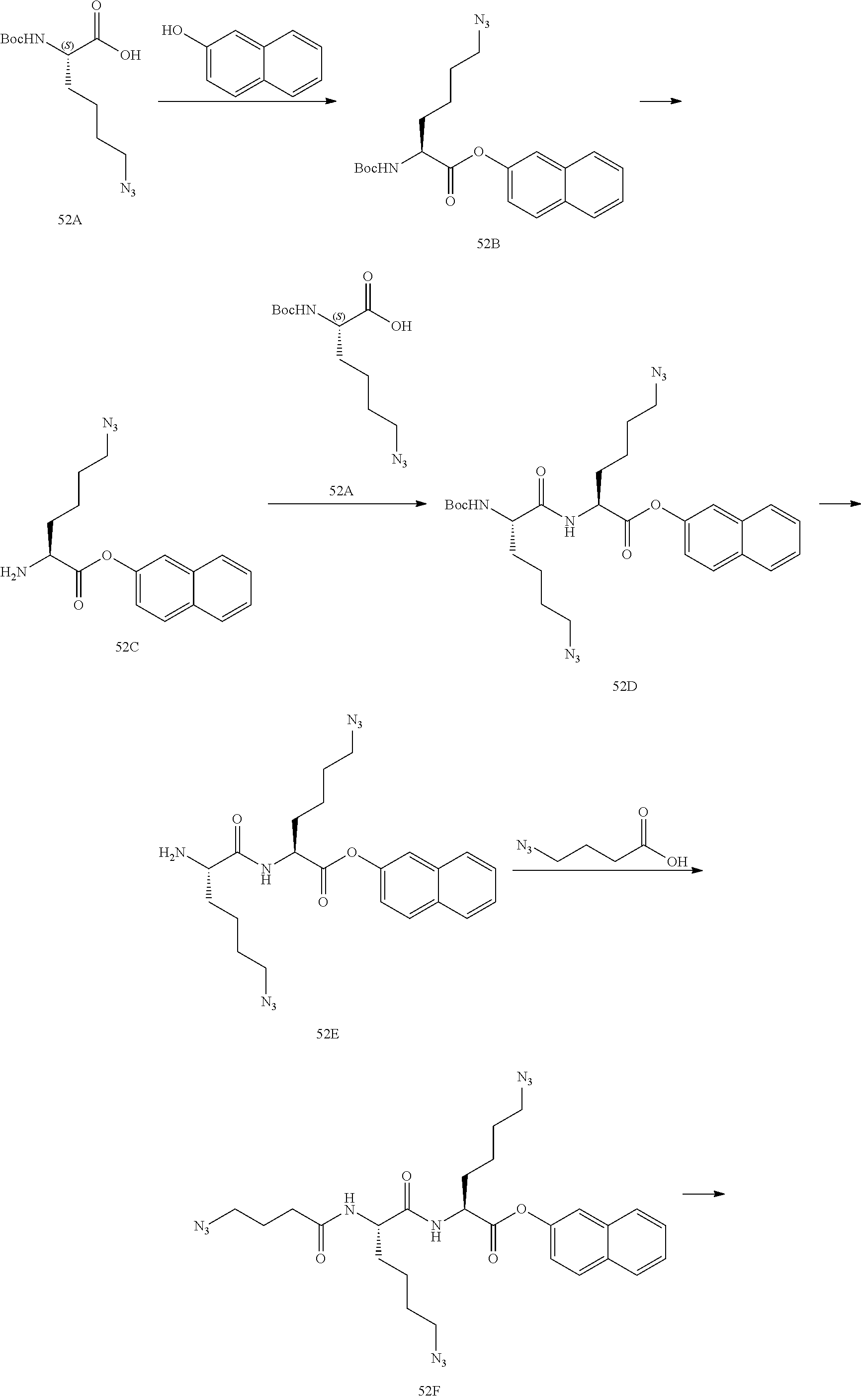

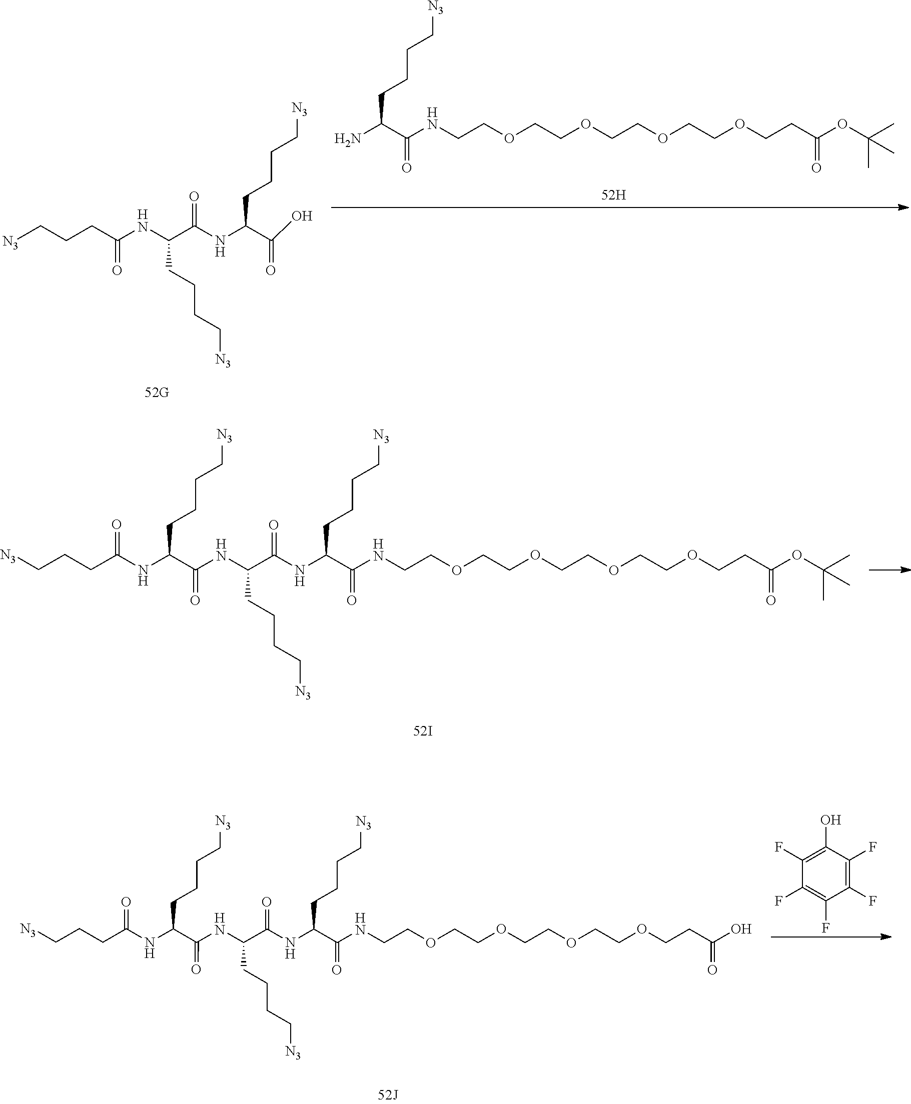 [SMILES: [N-]=[N+]=NCCCC[C@H](NC(=O)[C@H](CCCCN=[N+]=[N-])NC(=O)CCCN=[N+]=[N-])C(=O)O ; CC(C)(C)OC(=O)CCOCCOCCOCCOCCNC(=O)[C@@H](N)CCCCN=[N+]=[N-] ; CC(C)(C)OC(=O)CCOCCOCCOCCOCCNC(=O)[C@H](CCCCN=[N+]=[N-])NC(=O)[C@H](CCCCN=[N+]=[N-])NC(=O)[C@H](CCCCN=[N+]=[N-])NC(=O)CCCN=[N+]=[N-] ; [N-]=[N+]=NCCCC[C@H](NC(=O)CCCN=[N+]=[N-])C(=O)N[C@@H](CCCCN=[N+]=[N-])C(=O)N[C@@H](CCCCN=[N+]=[N-])C(=O)NCCOCCOCCOCCOCCC(=O)O ; Oc1c(F)c(F)c(F)c(F)c1F]

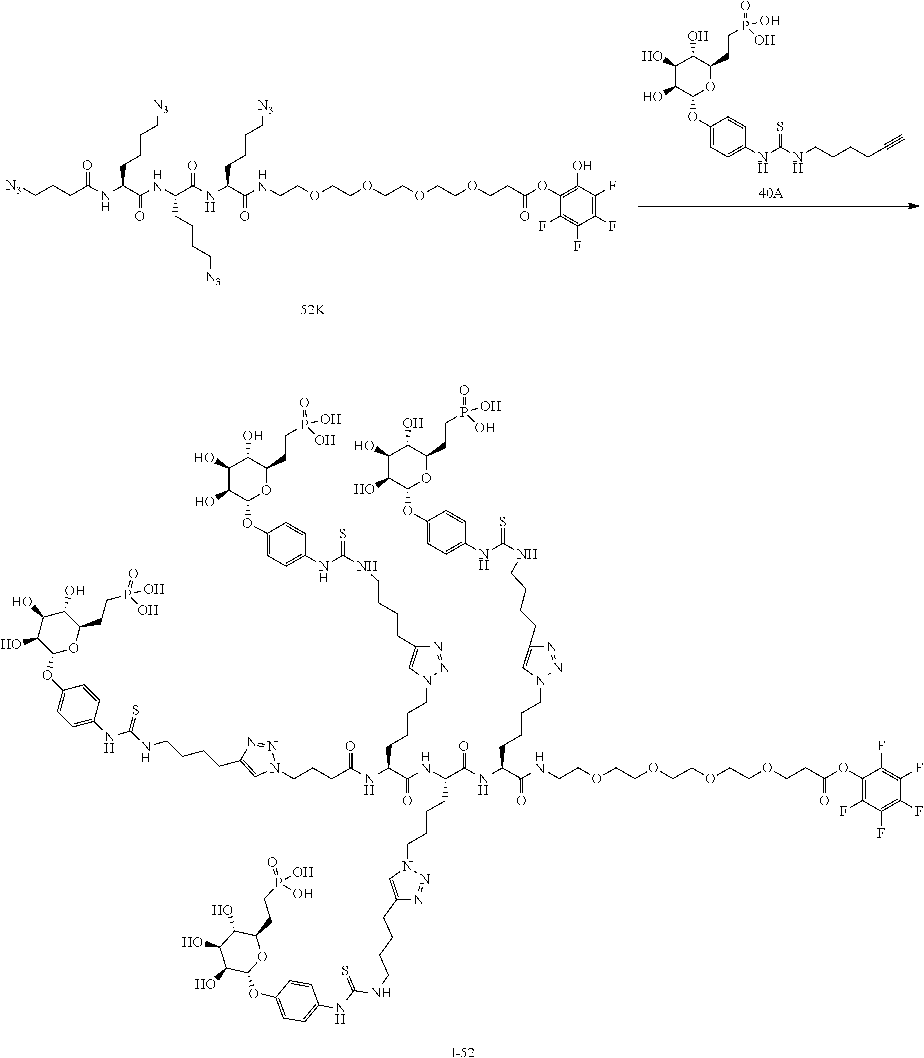

To a solution of N2-(tert-butoxycarbonyl)-N6-diazo-L-lysine (52A) (1.0 eq, 2.0 g, 7.34 mmol) in DCM (15.0 mL), naphthalen-2-ol (1.2 eq, 1.27 g, 8.81 mmol), (propan-2-yl)({[(propan-2-yl)imino]methylidene})amine (1.1 eq, 1.38 mL, 8.81 mmol) and N,N-dimethylpyridin-4-amine (0.1 eq, 0.897 g, 0.734 mmol) were added, and the reaction mixture was stirred at room temperature for 16 h. After completion, the reaction mixture was diluted with water and extract with DCM. The organic layer was dried over sodium sulfate, filtered, and concentrated under high vacuum to get crude. The crude was purified by flash column chromatography using 20% ethyl acetate in hexane to afford Compound 52B as off white solid. Yield: (2.50 g, 84%); LCMS m/z 399.2 [M+1]⁺.

To a solution of Compound 52B (1.0 eq, 2.5 g, 6.27 mmol) in DCM (5.00 mL), trifluoroacetic acid (3.0 mL) was added at room temperature. The resulting mixture was stirred at room temperature under nitrogen for 2 h. After completion, reaction mixture was concentrated and dried to afford Compound 52C as pale yellow viscous liquid. Yield: (1.80 g, 95%); LCMS m/z 299.15 [M+1]⁺.

To a solution of Compound 52C (1.0 eq, 1.80 g, 6.03 mmol) in THF (20 mL), Compound 52A (1.2 eq, 1.97 g, 7.24 mmol), HATU (1.5 eq, 3.44 g, 9.05 mmol) and ethylbis(propan-2-yl)amine (3.0 eq, 3.34 mL, 18.1 mmol) were added at room temperature. The resulting mixture was stirred at room temperature under nitrogen for 16 h. After completion, the reaction mixture was diluted with water and extract with ethyl acetate. The organic layer was dried over sodium sulfate, filtered, and concentrated under high vacuum to get crude. The crude was purified by flash column chromatography using 30% ethyl acetate in hexane to afford Compound 52D as pale yellow semi solid. Yield: (1.8 g, 55%); LCMS m/z 553.3 [M+1]⁺

To a solution of Compound 52D (1.0 eq, 1.50 g, 2.71 mmol) in DCM (10 mL), trifluoroacetic acid (5.0 mL) was added at room temperature. The resulting mixture was stirred at room temperature under nitrogen for 2 h. After completion, reaction mixture was concentrated and dried to afford Compound 52E as pale yellow viscous liquid. Yield: (1.0 g, 81%); LCMS m/z 453.01 [M+1]⁺.

To a solution of Compound 52E (1.0 eq, 1.0 g, 2.20 mmol) in DCM (10 mL), 4-azidobutanoic acid (1.0 eq, 0.285 g, 2.20 mmol), 1H-1,2,3-benzotriazol-1-ol (1.0 eq, 0.298 g, 2.20 mmol), ethylbis(propan-2-yl)amine (1.0 eq, 0.38 mL, 2.20 mmol), EDC.HCl (1.0 eq, 0.423 g, 2.20 mmol) were added at room temperature. The resulting mixture was stirred at room temperature under nitrogen for 16 h. After completion, the reaction mixture was diluted with water and extract with ethyl acetate. The organic layer was dried over sodium sulfate, filtered, and concentrated under high vacuum to get crude. The crude was purified by flash column chromatography using 40-50% ethyl acetate in hexane to afford Compound 52F as pale yellow semi solid. Yield: (0.60 g, 48%); LCMS m/z 564.3 [M+1]⁺.

To a solution of Compound 52F (1.0 eq, 0.60 g, 1.06 mmol) in THF (3.00 mL), methanol (3.00 mL) and water (0.5 mL), lithium hydroxide (3.0 eq, 0.105 g, 3.19 mmol) was added at room temperature. The resulting mixture was stirred at room temperature for 3 h. After completion, the reaction mixture was diluted with 1N HCl solution (pH=4) and extracted with ethyl acetate. The organic layer was dried over sodium sulfate, filtered, and concentrated under high vacuum to get crude. The crude was purified by flash column chromatography using 3-5% Methanol in DCM to afford Compound 52G as off white semi solid. Yield: (0.080 g, 17%); LCMS m/z 438.3 [M+1]⁺

To a solution of Compound 52G (1.0 eq, 0.080 g, 0.183 mmol) in tetrahydrofuran (1.0 mL) was added tert-butyl (S)-18-amino-22-azido-17-oxo-4,7,10,13-tetraoxa-16-azadocosanoate (52H) (1.0 eq, 0.087 g, 0.183 mmol) dissolved in tetrahydrofuran (1.0 mL), HATU (1.2 eq, 0.0834 g, 0.219 mmol) and ethylbis(propa-2-yl)amine (3.0 eq, 0.095 mL, 0.549 mmol) were added at room temperature, the reaction mixture stirred at room temperature for 3 h. After completion, the reaction mixture concentrated under reduced pressure to get crude. The crude was purified by flash column chromatography using 5-6% methanol in DCM to afford Compound 52I as pale yellow solid, yield: (0.130 g, 71.4%); LCMS m/z 895.5 [M+1]⁺.

To a stirred solution of Compound 52I (1.0 eq, 0.120 g, 0.134 mmol) in DCM (2.0 mL), 4 N HCl in 1,4 dioxane (2.0 mL) was added at room temperature, the resulting mixture was stirred at room temperature for 8 h. After completion, the reaction mixture concentrated under reduced pressure to get crude, the crude was triturated with n-pentane and dried to afford Compound 52J as pale yellow sticky solid. Yield: (0.10 g, 80%); LCMS m/z 839.2 [M+1]⁺.

To a solution of Compound 52J (1.0 eq, 0.100 g, 0.119 mmol) in DMF (2.0 mL) was cooled at 0° C., pentafluorophenol (5.0 eq, 0.109 g, 0.596 mmol) and DIC (5.0 eq, 0.094 mL, 0.596 mmol) were added at room temperature, the reaction mixture was stirred at room temperature for 2 h. After completion, reaction mixture was diluted with acetonitrile and purified by prep H PLC (70-75% acetonitrile in water with 0.1% TFA). Fractions containing the desired product were combined and lyophilized to dryness to afford Compound 52K as off white solid. Yield: 0.043 g, 36%; LC-MS m/z 1005.56 [M+1]⁺; ¹H-NMR (400 MHz, DMSO-d6) δ 8.05 (d, J=7.6 Hz, 1H), 7.98-7.95 (m, 2H), 7.79 (d, J=8.0 Hz, 1H), 4.26-4.19 (m, 3H), 3.78 (t, J=6.0 Hz, 2H), 3.54-3.49 (m, 12H), 3.40 (t, J=5.6 Hz, 2H), 3.32-3.15 (m, 12H), 3.03 (t, J=5.6 Hz, 2H), 2.22 (t, J=7.2 Hz, 2H), 1.77-1.71 (m, 2H), 1.70-1.63 (m, 3H), 1.51-1.49 (m, 10H), 1.31-1.29 (m, 7H).

Compound 40A (4.40 eq, 15.0 mg, 0.0306 mmol), Compound 52K (1.00 eq, 7.0 mg, 0.00697 mmol), and NMP (0.3 mL) were combined in a 1 dram vial with a stirbar, capped and stirred at room temperature. After 5 min [(CH₃CN)₄Cu]PF₆ (10.0 eq, 26.0 mg, 0.0697 mmol) was added. The resulting light yellow solution was capped and stirred at room temperature for 30 min. The reaction mixture slowly became more green-colored. The reaction mixture was diluted with a mixture of NMP and acetic acid, filtered, and purified via preparatory HPLC (15-50% acetonitrile in water with 0.1% TFA). Fractions containing the desired product were combined and lyophilized to dryness to afford Compound I-52 as a white solid. Yield: 14.4 mg, 70%; ¹H NMR (300 MHz, DMSO-d₆ with D₂O) δ 7.87-7.73 (m, 4H), 7.21-7.05 (m, 8H), 7.05-6.91 (m, 8H), 5.34 (s, 4H), 4.36-4.05 (m, 11H), 3.95-2.90 (m, 44H), 2.68-2.51 (m, 10H), 2.18-1.37 (m, 42H), 1.33-1.04 (m, 10H).

### Example 53: Synthesis of Compound I-53

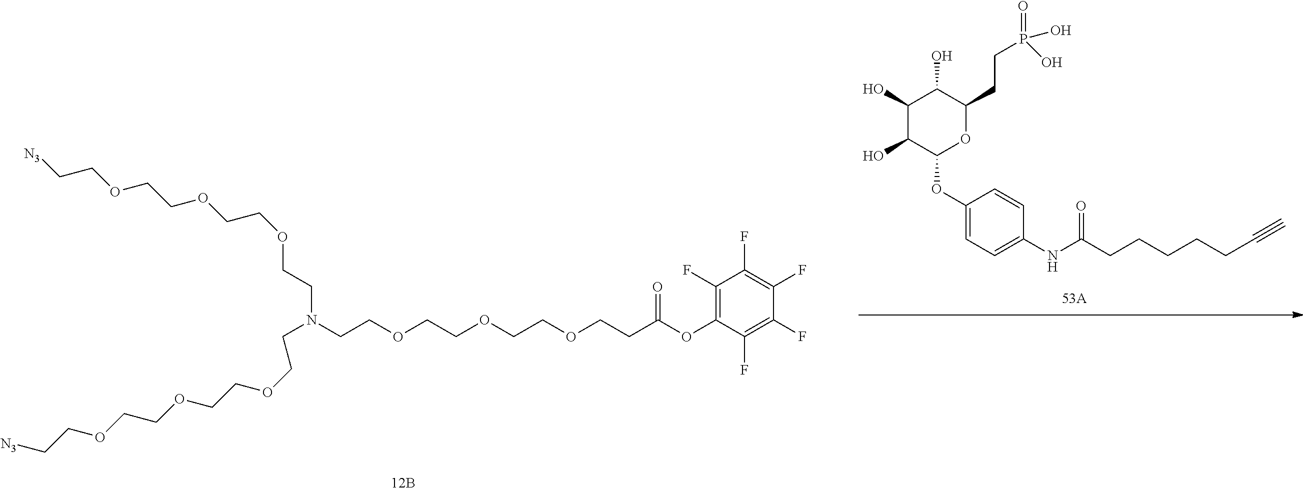 [SMILES: [N-]=[N+]=NCCOCCOCCOCCN(CCOCCOCCOCCN=[N+]=[N-])CCOCCOCCOCCC(=O)Oc1c(F)c(F)c(F)c(F)c1F ; C#CCCCCCC(=O)Nc1ccc(O[C@H]2O[C@H](CCP(=O)(O)O)[C@@H](O)[C@H](O)[C@@H]2O)cc1]

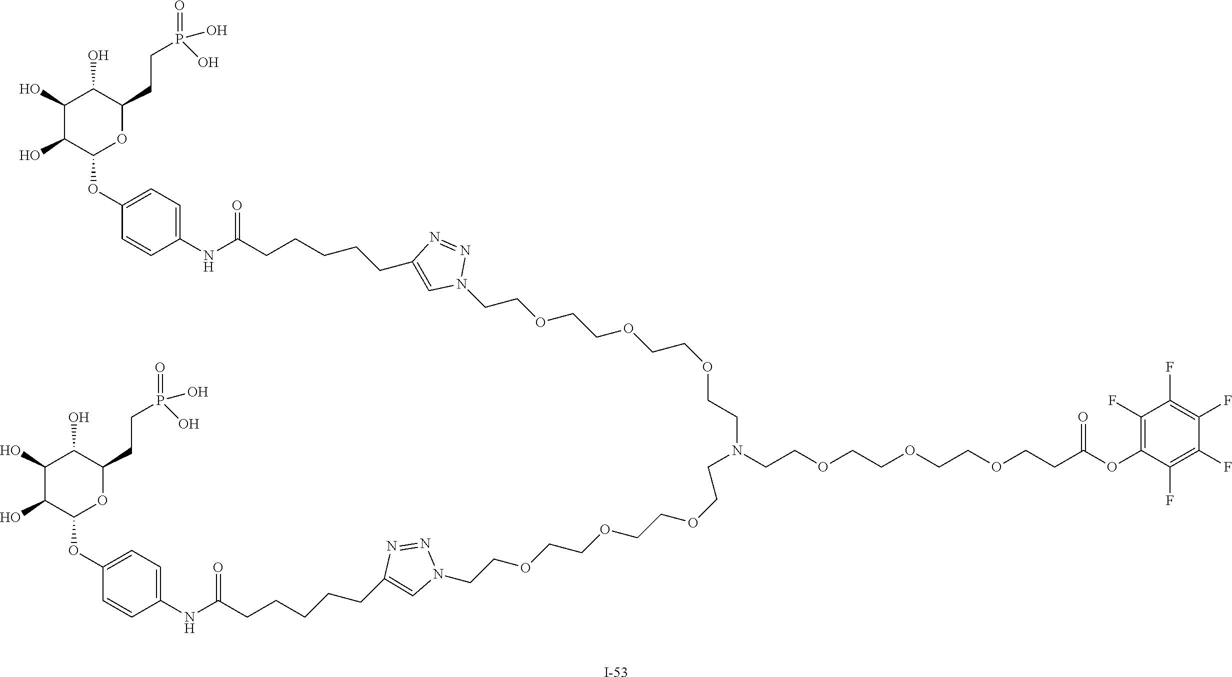

Compound I-53 is synthesized employing the procedures described for Compound I-52 using Compound 12B and (2-((2R,3S,4S,5S,6R)-3,4,5-trihydroxy-6-(4-(oct-7-ynamido)phenoxy)tetrahydro-2H-pyran-2-yl)ethyl)phosphonic acid (53A) in lieu of Compound 52K and Compound 40A.

### Synthesis of perfluorophenyl 1-azido-12-(2-(2-(2-(2-azidoethoxy)ethoxy)ethoxy)ethyl)-3,6,9,15,18,21-hexaoxa-12-azatetracosan-24-oate (Cpd. No. 12B)

To a stirred solution of 1-azido-12-(2-(2-(2-(2-azidoethoxy)ethoxy)ethoxy)ethyl)-3,6,9,15,18,21-hexaoxa-12-azatetracosan-24-oic acid (1, 1.00 eq, 500 mg, 0.802 mmol) in THF (2.5 mL) was added sequentially: N,N′dicyclohexylcarbodiimide (1.50 eq, 248 mg, 1.20 mmol), a solution of 2,3,4,5,6-pentafluorophenol (1.70 eq, 251 mg, 1.36 mmol) in THF (1 mL), and then 4-dimethylaminopyridine (0.0300 eq, 2.9 mg, 0.0241 mmol). The resulting mixture was capped and stirred at room temperature for 17 h. The reaction mixture was diluted with diethyl ether and filtered. The filtrate was concentrated on a rotary evaporator. The residue was taken up in dichloromethane and purified via silica gel chromatography (0-100% acetonitrile in dichloromethane) to afford perfluorophenyl 1-azido-12-(2-(2-(2-(2-azidoethoxy)ethoxy)ethoxy)ethyl)-3,6,9,15,18,21-hexaoxa-12-azatetracosan-24-oate (2) as a yellow oil. Yield: 258 mg, 41%; LCMS m/z 790.7 [M+1]+; ¹H NMR (300 MHz, Chloroform-d) δ 3.87 (t, J=6.2 Hz, 2H), 3.74-3.56 (m, 16H), 3.39 (t, J=5.1 Hz, 4H), 2.94 (t, J=6.2 Hz, 2H).

### Synthesis of Cpd. No. I-53

A solution of (2-((2R,3S,4S,5S,6R)-3,4,5-trihydroxy-6-(4-(oct-7-ynamido)phenoxy)tetrahydro-2H-pyran-2-yl)ethyl)phosphonic acid (2a, 2.20 eq) in NMP is added to perfluorophenyl 1-azido-12-(2-(2-(2-(2-azidoethoxy)ethoxy)ethoxy)ethyl)-3,6,9,15,18,21-hexaoxa-12-azatetracosan-24-oate (2, 1.00 eq) in a 1 dram vial with a stirbar. The resulting solution is stirred and tetrakis(acetonitrile)copper(I) hexafluorophosphate (5.00 eq) is added. The resulting solution is capped and stirred at room temperature for 25 min. The reaction mixture is diluted with acetic acid, filtered, and purified via preparatory HPLC. Fractions containing the desired product are combined and lyophilized to dryness to afford (2-((2R,3S,4S,5S,6R)-3,4,5-trihydroxy-6-(4-(6-(1-(24-oxo-12-(2-(2-(2-(2-(4-(6-oxo-6-((4-(((2R,3S,4S,5S,6R)-3,4,5-trihydroxy-6-(2-phosphonoethyl)tetrahydro-2H-pyran-2-yl)oxy)phenyl)amino)hexyl)-1H-1,2,3-triazol-1-yl)ethoxy)ethoxy)ethoxy)ethyl)-24-(perfluorophenoxy)-3,6,9,15,18,21-hexaoxa-12-azatetracosyl)-1H-1,2,3-triazol-4-yl)hexanamido)phenoxy)tetrahydro-2H-pyran-2-yl)ethyl)phosphonic acid (Cpd. No. I-53).

### Example 54: Synthesis of Compound I-54

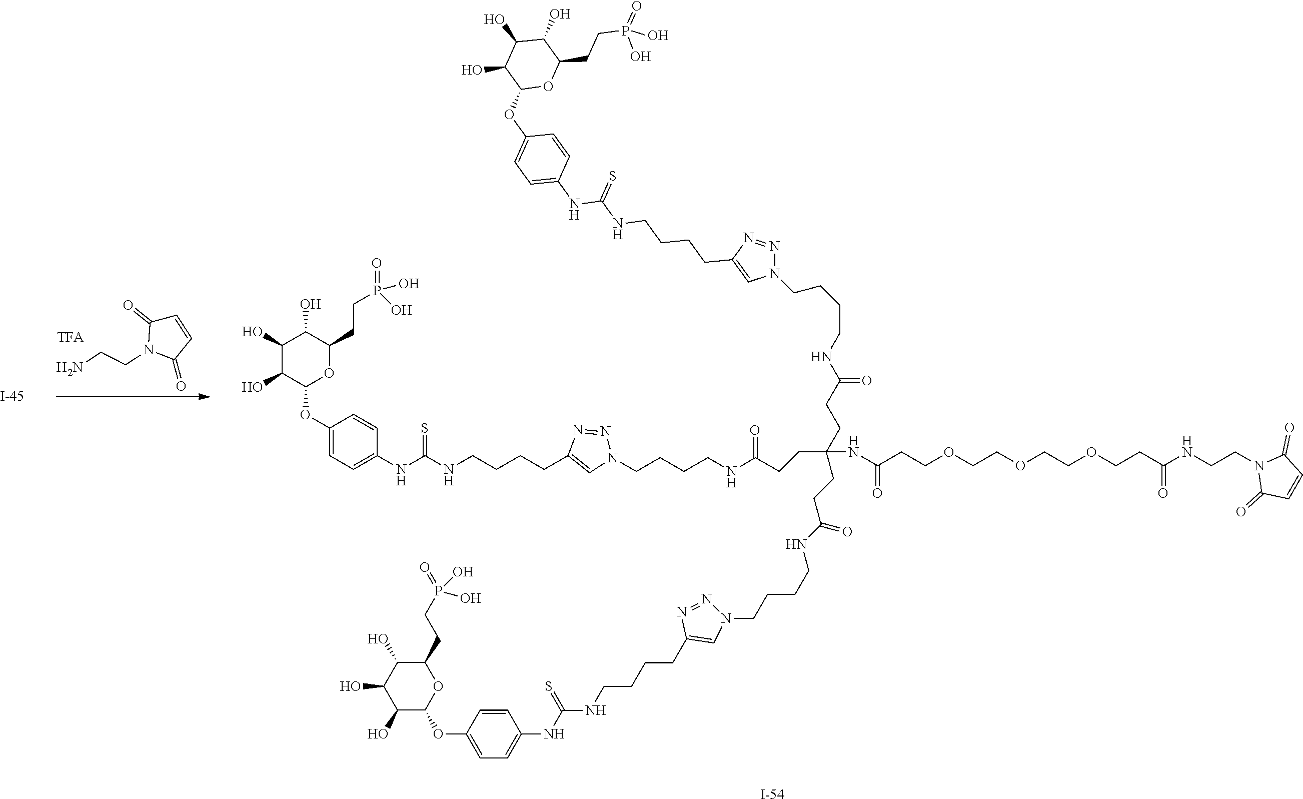

Compound I-54 is synthesized employing the procedures described for Compound I-50 using Compound I-45 in lieu of Compound I-38.

To 1-45 (1.00 eq) in a vial with a stirbar is added a solution of 1-(2-aminoethyl)-1H-pyrrole-2,5-dione TFA salt (1, 1.15 eq) and N,N-diisopropylethylamine (3.00 eq) in NMP. The resulting solution is capped and stirred at room temperature for 30 min. The reaction mixture is diluted with acetic acid, filtered, and purified via preparatory HPLC. Fractions containing the desired product are combined and lyophilized to dryness to afford Cpd. No. 1-54.

### Example 55: Synthesis of Compound I-55

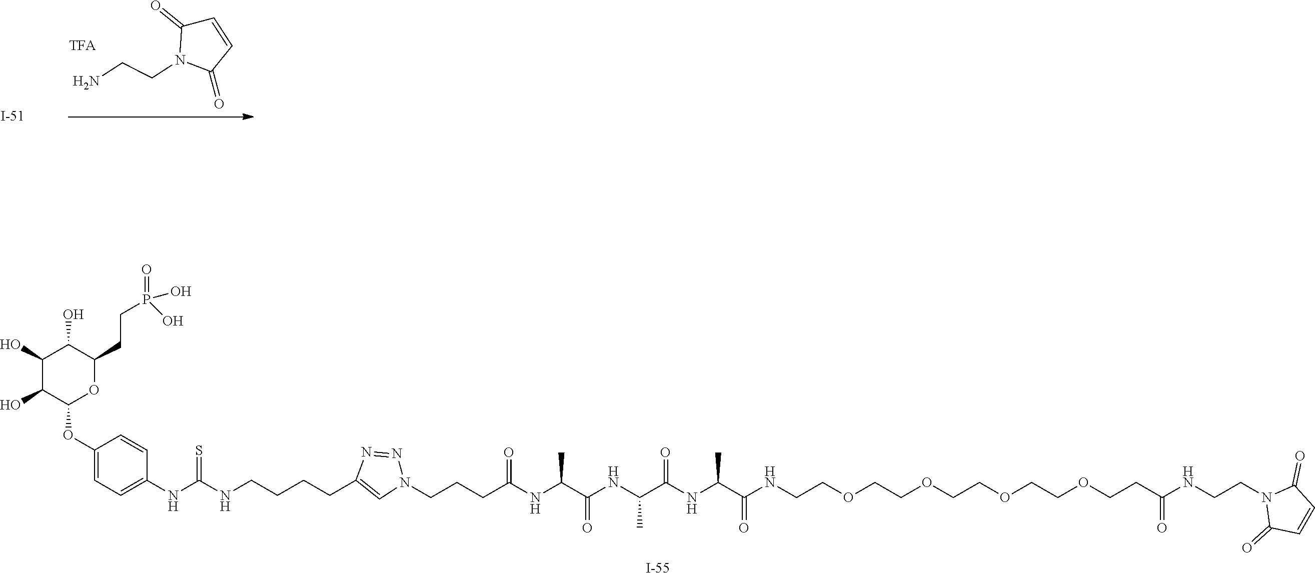

Compound I-55 is synthesized employing the procedures described for Compound I-50 using Compound I-52 in lieu of Compound I-38.

To (2-((2R,3S,4S,5S,6R)-3,4,5-trihydroxy-6-(4-(3-(4-(1-((18S,21S,24S)-18,21,24-trimethyl-1,17,20,23,26-pentaoxo-1-(perfluorophenoxy)-4,7,10,13-tetraoxa-16,19,22,25-tetraazanonacosan-29-yl)-1H-1,2,3-triazol yl)butyl)thioureido)phenoxy)tetrahydro-2H-pyran-2-yl)ethyl)phosphonic acid (I-51, 1.00 eq) in a vial with a stirbar is added a solution of 1-(2-aminoethyl)-1H-pyrrole-2,5-dione TFA salt (1, 1.15 eq) and N,N-diisopropylethylamine (3.00 eq) in NMP. The resulting solution is capped and stirred at room temperature for 20 min. The reaction mixture is diluted with acetic acid, filtered, and purified via preparatory HPLC. Fractions containing the desired product are combined and lyophilized to dryness to afford (2-((2R,3S,4S,5S,6R)-6-(4-(3-(4-(1-((21S,24S,27S)-1-(2,5-dioxo-2,5-dihydro-1H-pyrrol-1-yl)-21,24,27-trimethyl-4,20,23,26,29-pentaoxo-7,10,13,16-tetraoxa-3,19,22,25,28-pentaazadotriacontan-32-yl)-1H-1,2,3-triazol-4-yl)butyl)thioureido)phenoxy)-3,4,5-trihydroxytetrahydro-2H-pyran-2-yl)ethyl)phosphonic acid (Cpd. No. I-55).

### Example 56: Synthesis of Compound I-56

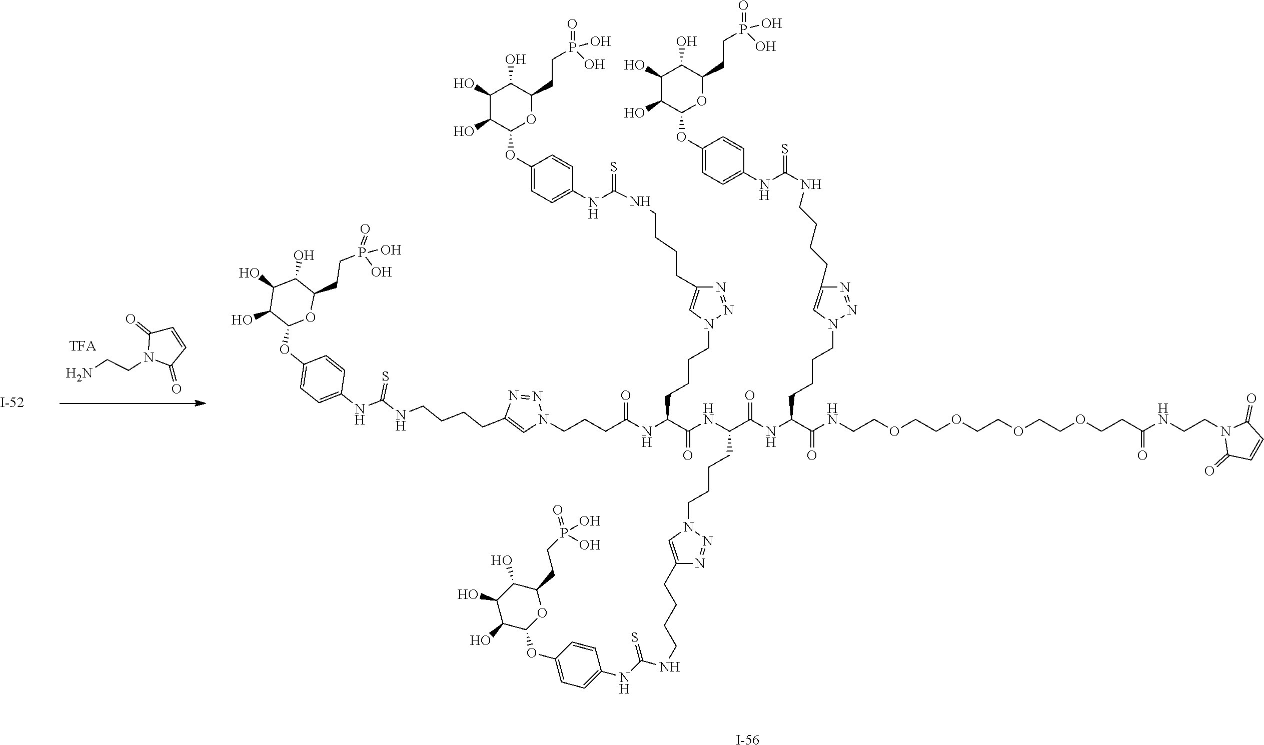

Compound I-56 is synthesized employing the procedures described for Compound I-50 using Compound I-52 in lieu of Compound I-38.

To (2-((2R,3S,4S,5S,6R)-3,4,5-trihydroxy-6-(4-(3-(4-(1-((18S,21S,24S)-1,17,20,23,26-pentaoxo-1-(perfluorophenoxy)-18,21,24-tris(4-(4-(4-(3-(4-(((2R,3S,4S,5S,6R)-3,4,5-trihydroxy-6-(2-phosphonoethyl)tetrahydro-2H-pyran yl)oxy)phenyl)thioureido)butyl)-1H-1,2,3-triazol-1-yl)butyl)-4,7,10,13-tetraoxa-16,19,22,25-tetraazanonacosan-29-yl)-1H-1,2,3-triazol-4-yl)butyl)thioureido)phenoxy)tetrahydro-2H-pyran-2-yl)ethyl)phosphonic acid (I-52, 1.00 eq) in a vial with a stirbar is added a solution of 1-(2-aminoethyl)-1H-pyrrole-2,5-dione TFA salt (1, 1.15 eq) and N,N-diisopropylethylamine (3.00 eq) in NMP. The resulting solution is capped and stirred at room temperature for 20 min. The reaction mixture is diluted with acetic acid, filtered, and purified via preparatory HPLC. Fractions containing the desired product are combined and lyophilized to dryness to afford (2-((2R,3S,4S,5S,6R)-6-(4-(3-(4-(1-((21S,24S,27S)-1-(2,5-dioxo-2,5-dihydro-1H-pyrrol-1-yl)-4,20,23,26,29-pentaoxo-21,24,27-tris(4-(4-(4-(3-(4-(((2R,3S,4S,5S,6R)-3,4,5-trihydroxy-6-(2-phosphonoethyl)tetrahydro-2H-pyran-2-yl)oxy)phenyl)thioureido)butyl)-1H-1,2,3-triazol-1-yl)butyl)-7,10,13,16-tetraoxa-3,19,22,25,28-pentaazadotriacontan-32-yl)-1H-1,2,3-triazol-4-yl)butyl)thioureido)phenoxy)-3,4,5-trihydroxytetrahydro-2H-pyran-2-yl)ethyl)phosphonic acid (Cpd. No. I-56).

### Example 57: Synthesis of Compound I-57

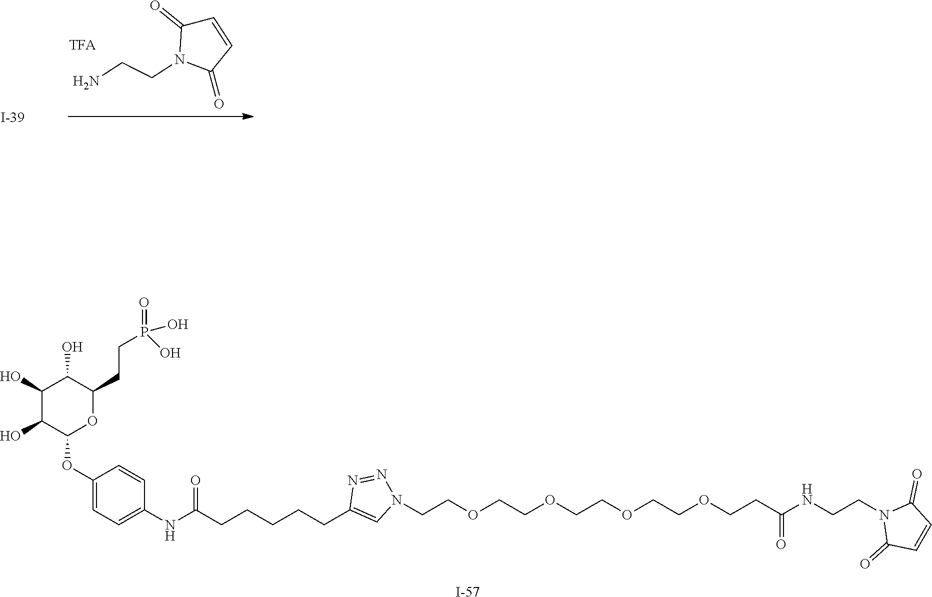

Compound I-57 is synthesized employing the procedures described for Compound I-50 using Compound I-39 in lieu of Compound I-38.

### Synthesis of (2-((2R,3S,4S,5S,6R)-6-(4-(6-(1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)hexanamido)phenoxy)-3,4,5-trihydroxytetrahydro-2H-pyran-2-yl)ethyl)phosphonic acid (Cpd. No. I-57)

To (2-((2R,3S,4S,5S,6R)-3,4,5-trihydroxy-6-(4-(6-(1-(15-oxo-15-(perfluorophenoxy)-3,6,9,12-tetraoxapentadecyl)-1H-1,2,3-triazol-4-yl)hexanamido)phenoxy)tetrahydro-2H-pyran-2-yl)ethyl)phosphonic acid (I-39, 1.00 eq) in a vial with a stirbar is added a solution of 1-(2-aminoethyl)-1H-pyrrole-2,5-dione TFA salt (1, 1.15 eq) and N,N-diisopropylethylamine (3.00 eq) in NMP. The resulting solution is capped and stirred at room temperature for 30 min. The reaction mixture is diluted with acetic acid, filtered, and purified via preparatory HPLC. Fractions containing the desired product are combined and lyophilized to dryness to afford (2-((2R,3S,4S,5S,6R)-6-(4-(6-(1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)hexanamido)phenoxy)-3,4,5-trihydroxytetrahydro-2H-pyran-2-yl)ethyl)phosphonic acid (Cpd. No. I-57).

### Example 58: Synthesis of Compound I-58

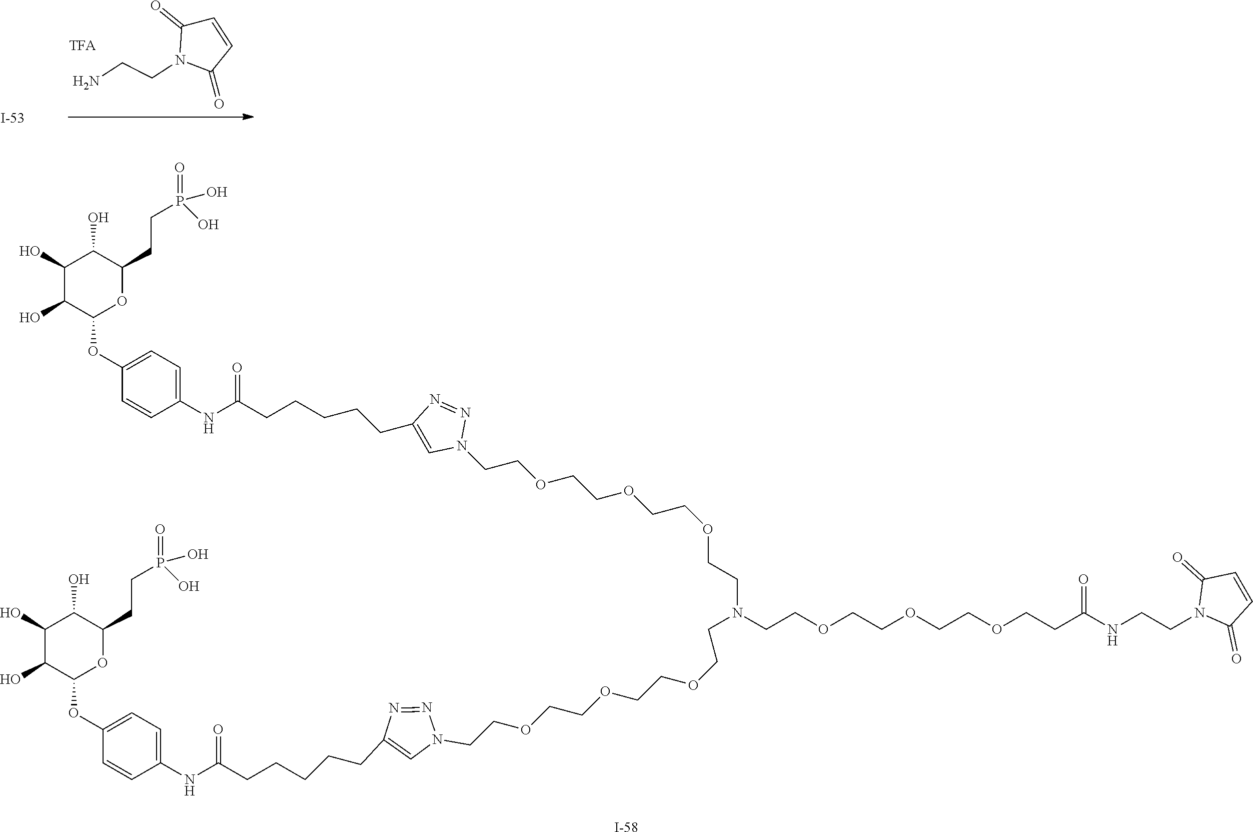

Compound I-58 was synthesized employing the procedures described for Compound I-50 using Compound I-53 in lieu of Compound I-38.

To (2-((2R,3S,4S,5S,6R)-3,4,5-trihydroxy-6-(4-(6-(1-(24-oxo-12-(2-(2-(2-(2-(4-(6-oxo-6-((4-(((2R,3S,4S,5S,6R)-3,4,5-trihydroxy-6-(2-phosphonoethyl)tetrahydro-2H-pyran-2-yl)oxy)phenyl)amino)hexyl)-1H-1,2,3-triazol-1-yl)ethoxy)ethoxy)ethoxy)ethyl)-24-(perfluorophenoxy)-3,6,9,15,18,21-hexaoxa-12-azatetracosyl)-1H-1,2,3-triazol-4-yl)hexanamido)phenoxy)tetrahydro-2H-pyran-2-yl)ethyl)phosphonic acid (I-53, 1.00 eq) in a vial with a stirbar is added a solution of 1-(2-aminoethyl)-1H-pyrrole-2,5-dione TFA salt (1, 1.15 eq) and N,N-diisopropylethylamine (3.00 eq) in NMP. The resulting solution is capped and stirred at room temperature for 30 min. The reaction mixture is diluted with acetic acid, filtered, and purified via preparatory HPLC. Fractions containing the desired product are combined and lyophilized to dryness to afford (2-((2R,3S,4S,5S,6R)-6-(4-(6-(1-(27-(2,5-dioxo-2,5-dihydro-1H-pyrrol-1-yl)-24-oxo-12-(2-(2-(2-(2-(4-(6-oxo-64(4-(((2R,3S,4S,5S,6R)-3,4,5-trihydroxy-6-(2-phosphonoethyl)tetrahydro-2H-pyran-2-yl)oxy)phenyl)amino)hexyl)-1H-1,2,3-triazol-1-yl)ethoxy)ethoxy)ethoxy)ethyl)-3,6,9,15,18,21-hexaoxa-12,25-diazaheptacosyl)-1H-1,2,3-triazol-4-yl)hexanamido)phenoxy)-3,4,5-trihydroxytetrahydro-2H-pyran-2-yl)ethyl)phosphonic acid (Cpd. No. I-58).

### Example 59: Synthesis of Compound I-59

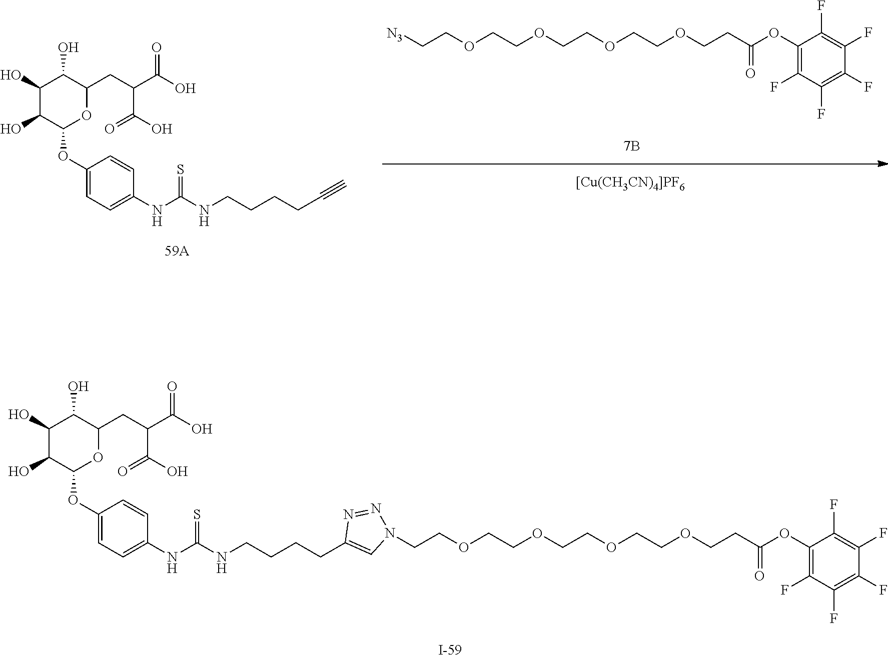

To a glass vial purged with nitrogen was added Compound 7B (1.30 eq, 24.0 mg, 0.0524 mmol), and then added NMP (0.90 mL) followed by [(CH₃CN)₄Cu]PF₆ (2.50 eq, 37.6 mg, 0.101 mmol) with stirring. Compound 59A (1.00 eq, 20.0 mg, 0.0403 mmol) was added. The resulting light yellow solution was capped and stirred at room temperature. LCMS at 15 min shows complete conversion. The reaction mixture was diluted with mixture of NMP, ethanol, and acetic acid, filtered, and purified via preparatory HPLC (10-50% acetonitrile in water with 0.1% TFA) 20 min run. Fractions containing the desired product were combined and lyophilized to dryness to afford Compound I-59 (18 mg, 47% yield) as a white solid. LCMS m/z 954.5 [M+1]⁺; ¹H NMR (300 MHz, DMSO-d₆) δ 7.76 (s, 1H), 7.16 (d, J=8.2 Hz, 2H), 6.94 (d, J=8.4 Hz, 2H), 5.27 (s, 1H), 4.41 (t, J=4.8 Hz, 2H), 3.84-2.81 (m, 25H), 2.65-2.20 (m, 3H), 1.75-1.41 (m, 5H).

### Example 60: Synthesis of Compound I-60

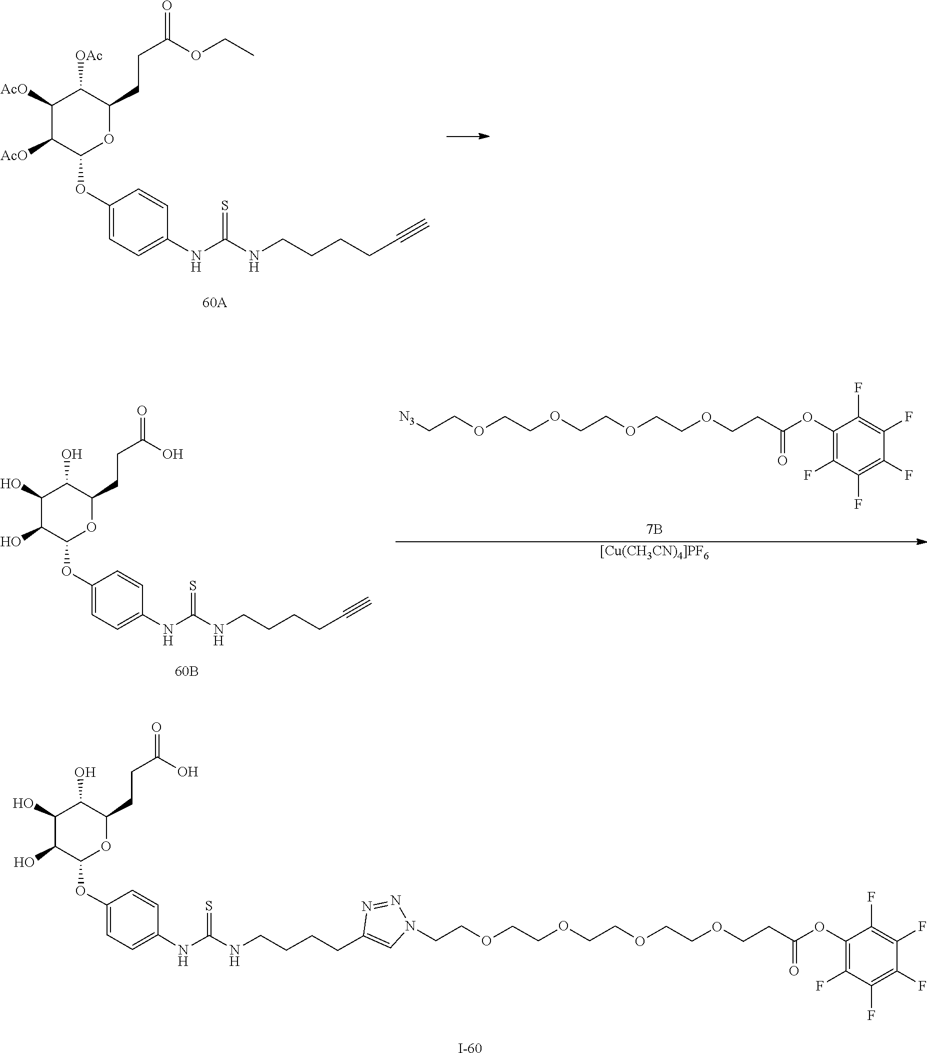

To a round bottom flask was added (2R,3R,4S,5S,6R)-2-(3-ethoxy-3-oxopropyl)-6-(4-(3-(hex-5-yn-1-yl)thioureido)phenoxy)tetrahydro-2H-pyran-3,4,5-triyl triacetate (60A) (1.00 eq, 244 mg, 0.491 mmol) and THF (4 mL). To the stirring solution was added 3 M LiOH aq. (10.4 eq, 1.7 mL, 5.10 mmol). The reaction solution was allowed to stir at room temperature for 2 hrs. The reaction solution was diluted with EtOAc (30 mL) and aq. NH₄Cl. The organic phase was partitioned, washed with brine, dried over Na₂SO₄, filtered and concentrated in vacuo. The product Compound 60B (210 mg, 91% yield) was used in the next step with no additional purification. LC-MS m/z 453.6 [M+1]+.

To a glass vial purged with nitrogen was added Compound 7B (1.30 eq, 75.9 mg, 0.166 mmol). To the vial was added NMP (0.90 mL) followed by [(CH₃CN)₄Cu]PF₆ (2.50 eq, 119 mg, 0.319 mmol) with stirring. Compound 60B (1.00 eq, 58.0 mg, 0.128 mmol) was added and the resulting light yellow solution was capped and stirred at room temperature. After 30 min, the reaction was found complete by LCMS. The reaction mixture was diluted with mixture of NMP, ethanol, and acetic acid, filtered, and purified via preparatory HPLC (10-50% acetonitrile in water with 0.1% TFA) over a 20 min run. Fractions containing the desired product were combined and lyophilized to dryness to afford Compound I-60 (44 mg, 38 yield) as a white solid. LCMS m/z 910.6 [M+H]+.

### Example 61: Synthesis of Compound I-61

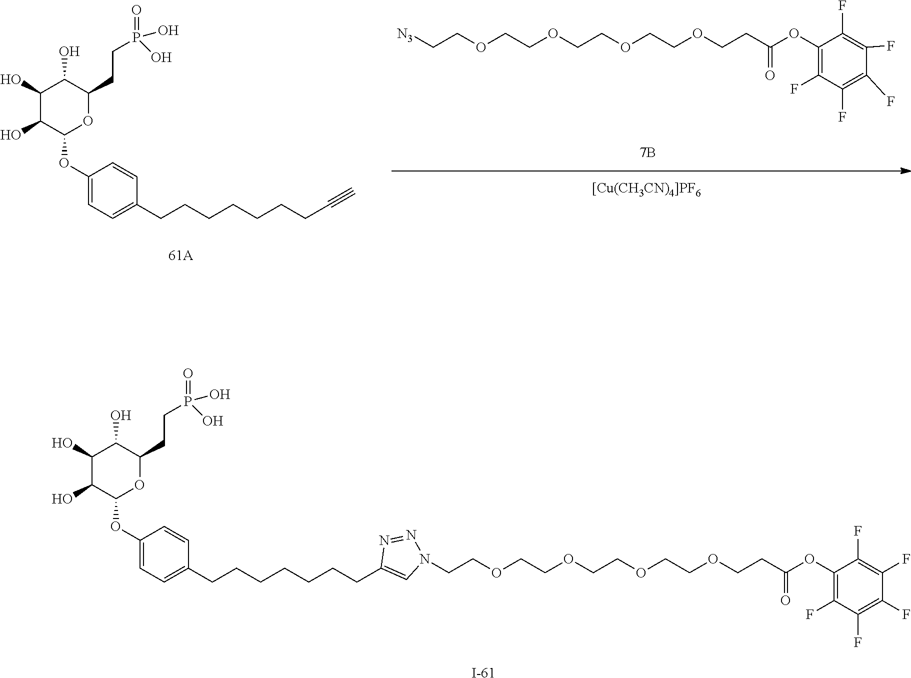

Compound I-61 is synthesized employing the procedures described for Compound I-60 using Compound 61A in lieu of Compound 60B.

To (2-((2R,3S,4S,5S,6R)-3,4,5-trihydroxy-6-(4-(non-8-yn-1-yl)phenoxy)tetrahydro-2H-pyran-2-yl)ethyl)phosphonic acid (1.00 eq, 30.7 mg, 0.0672 mmol) in a 1 dram vial with a stirbar was added a solution of perfluorophenyl 1-azido-3,6,9,12-tetraoxapentadecan-15-oate (1, 1.20 eq, 36.9 mg, 0.0806 mmol) in NMP (0.5 mL) followed by tetrakis(acetonitrile)copper(I) hexafluorophosphate (2.50 eq, 62.6 mg, 0.168 mmol). The resulting clear yellow solution was capped and stirred at room temperature for 25 min (slowly became more green colored). The reaction mixture was diluted with mixture of NMP, ethanol, and acetic acid, filtered, and purified via preparatory HPLC (15-60% acetonitrile in water with 0.1% TFA). Fractions containing the desired product were combined and lyophilized to dryness to afford (2-((2R,3S,4S,5S,6R)-3,4,5-trihydroxy-6-(4-(7-(1-(15-oxo-15-(perfluorophenoxy)-3,6,9,12-tetraoxapentadecyl)-1H-1,2,3-triazol-4-yl)heptyl)phenoxy)tetrahydro-2H-pyran-2-yl)ethyl)phosphonic acid (Cpd. No. I-61) as a white solid. Yield: 33.8 mg, 55%; LCMS m/z 914.5 [M+1]+; ¹H NMR (300 MHz, DMSO-d₆ with D₂O) δ 7.74 (s, 1H), 7.06 (d, J=7.7 Hz, 2H), 6.89 (d, J=8.6 Hz, 2H), 5.27 (s, 1H), 4.40 (t, J=4.8 Hz, 2H), 3.82-3.67 (m, 5H), 3.61 (d, J=8.4 Hz, 1H), 3.54-3.24 (m, 14H), 2.93 (t, J=6.0 Hz, 2H), 2.59-2.37 (m, 4H), 1.95-1.79 (m, 1H), 1.63-1.38 (m, 6H), 1.31-1.06 (m, 7H).

### Example 62: Synthesis of Compound I-62

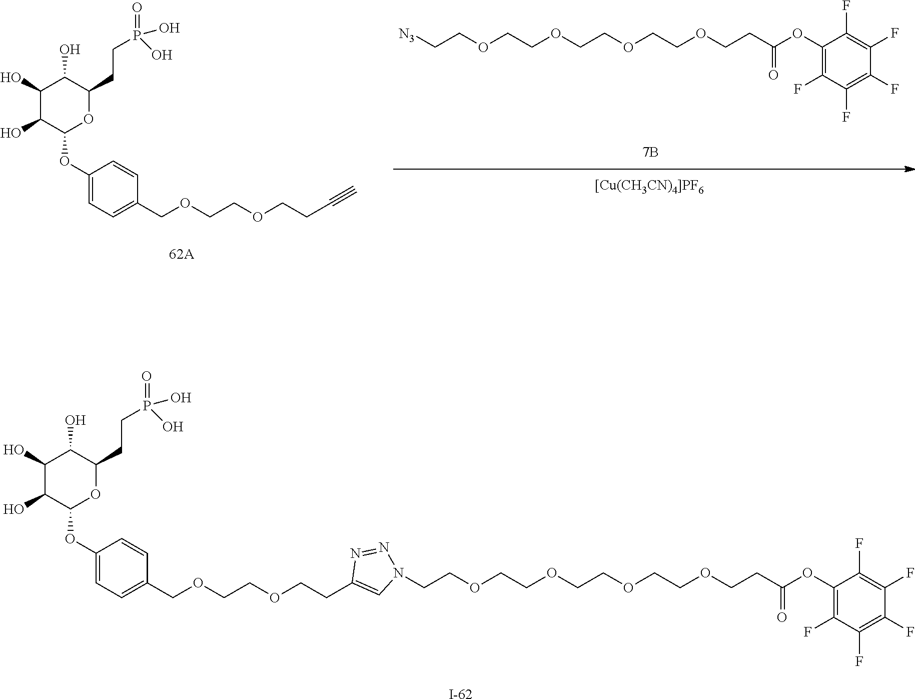

Compound I-62 is synthesized employing the procedures described for Compound I-60 using Compound 62A in lieu of Compound 60B.

### Example 63: Synthesis of Compound I-63

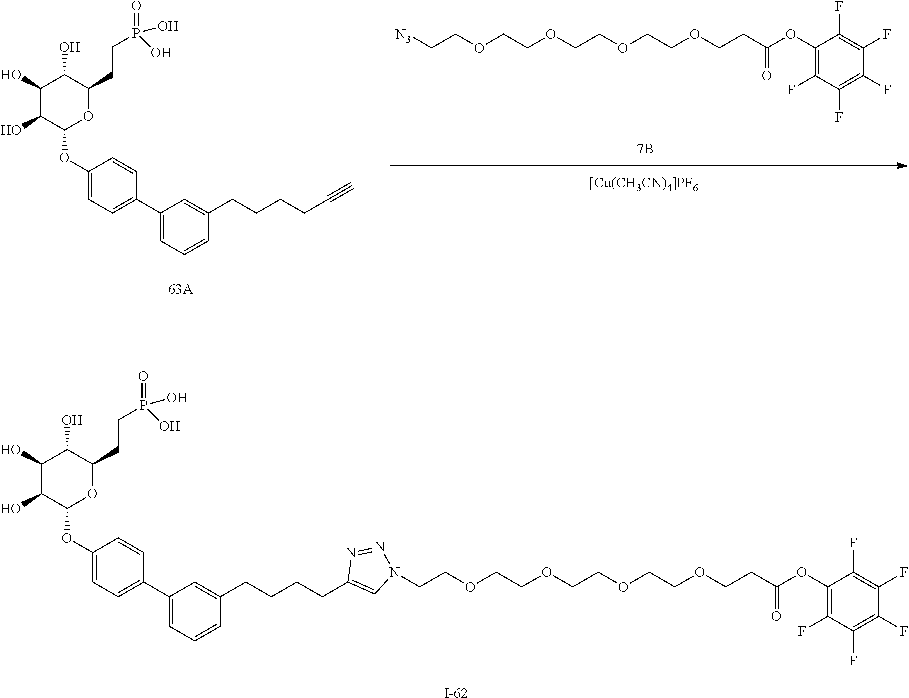

Compound I-63 is synthesized employing the procedures described for Compound I-60 using Compound 63A in lieu of Compound 60B.

### Synthesis of (2-((2R,3S,4S,5S,6R)-6-((3′-(hex-5-yn-1-yl)-[1,1′-biphenyl]-4-yl)oxy)-3,4,5-trihydroxytetrahydro-2H-pyran-2-yl)ethyl)phosphonic acid (Compound 63A)

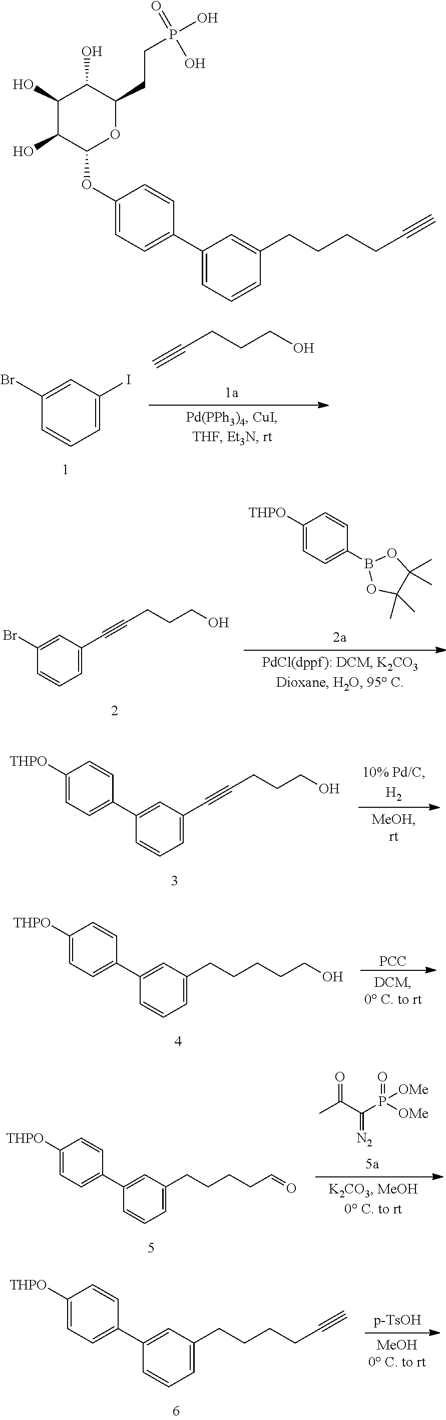

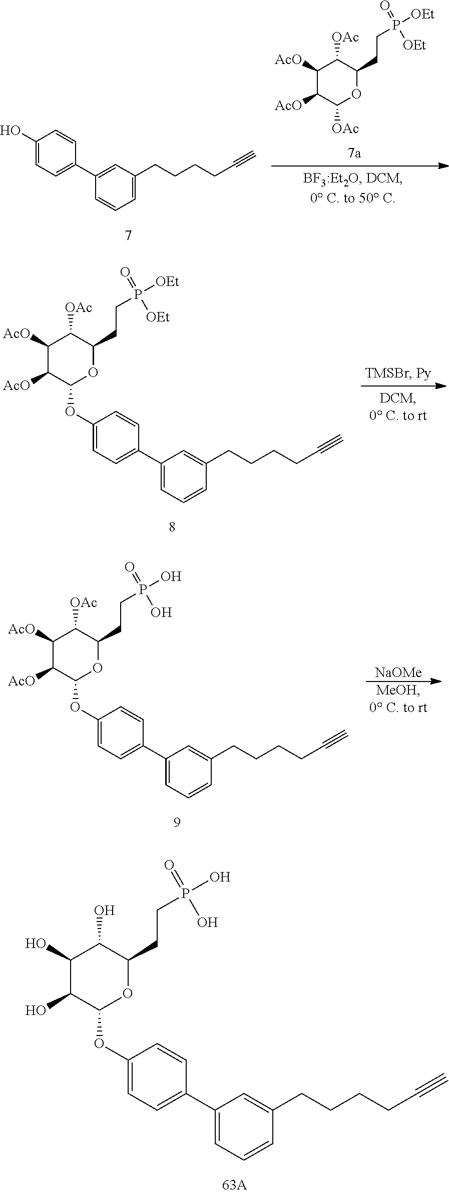

### Synthesis of 5-(3-bromophenyl)pent-4-yn-1-ol (2)

To a solution of 1-bromo-3-iodobenzene (1, 16.8 g, 1.0 eq, 59.4 mmol) in tetrahydrofuran (90 mL) pent-4-yn-1-ol (1a, 5 g, 1.0 eq, 59.4 mmol), triethylamine (25.1 mL, 3.0 eq, 178 mmol) and copper(I) iodide (1.13 g, 0.1 eq, 5.94 mmol) were added and reaction mixture purged with flow of argon gas for 15 minutes. Tetrakis(triphenylphosphane) palladium (3.43 g, 0.05 eq, 2.97 mmol) was then added to reaction mixture and reaction mixture stirred at room temperature for 16 h. Reaction mixture partitioned in between ethyl acetate and water. Ethyl acetate layer separated, washed with water, brine, dried over anhydrous sodium sulphate, filtered and concentrated under reduced pressure to get crude product. crude product obtained was purified by flash column chromatography using silica gel column and eluting product in 10 to 30% ethyl acetate in hexane as eluents. Desired fractions were concentrated under reduced pressure to afford 5-(3-bromophenyl) pent-4-yn-1-01 (2) as brownish sticky gum. Yield: 14.0 g, 98.5%; LC-MS m/z 239.26 [M+1]⁺

### Synthesis of 5-(4′-((tetrahydro-2H-pyran-2-yl)oxy)-[1,1′-biphenyl]-3-yl)pent-4-yn-1-ol(3)

To a solution of 5-(3-bromophenyl)pent-4-yn-1-ol (2, 6.95 g, 1.3 eq, 29.1 mmol) in 1,4-dioxane (120 mL) was added 4,4,5, 5-tetramethyl-2-[4-(oxan-2-yloxy)phenyl]-1,3,2-dioxaborolane (2a, 6.80 g, 1.0 eq, 22.4 mmol) and potassium carbonate solution (9.27 g, 3 eq, 67.2 mmol) in water (30.0 ml) and reaction mixture purged with argon gas for 15 minutes. [1,1′-Bis(diphenylphosphino)ferrocene]dichloropalladium(II):DCM (0.912 g, 0.05 eq., 1.12 mmol) was then added and reaction mixture stirred at 95° C. for 4 h. Reaction mixture quenched by addition of water and extracted with ethyl acetate. Ethyl acetate layer dried over anhydrous sodium sulphate and concentrated under reduced pressure to get crude product. Crude product obtained was purified by flash chromatography using silica gel column and eluting product in 10 to 30% Ethyl acetate in hexane as eluents. Desired fractions were concentrated under reduced pressure to afford 5-(4′-((tetrahydro-2H-pyran yl) oxy)-[1,1′-biphenyl]-3-yl) pent-4-yn-1-ol (3) as colorless sticky gum. Yield: 4.90 g, 65.15%; LC-MS m/z 337.21 [M+1]⁺

### Synthesis of 5-(4′-((tetrahydro-2H-pyran-2-yl)oxy)-[1,1′-biphenyl]-3-yl)pentan-1-ol (4)

To a solution of 5-(4′-((tetrahydro-2H-pyran-2-yl)oxy)-[1,1′-biphenyl]-3-yl)pent-4-yn-1-ol (3, 0.25 g, 0.74 mmol) in Methanol (10 mL) was added 10% palladium on carbon (0.080 g), Reaction mixture then stirred at room temperature under hydrogen atmosphere for 16 h. Reaction mixture filtered over celite pad, Filtrate obtained was concentrated under reduced pressure to afford 5-(4′-((tetrahydro-2H-pyran-2-yl)oxy)-[1,1′-biphenyl]-3-yl)pentan-1-01 (4) as colorless sticky gum. Yield: 0.24 g, 94.86%; LC-MS m/z 339.17 [M−1]⁻

### Synthesis of 5-(4′-((tetrahydro-2H-pyran-2-yl)oxy)-[1,1′-biphenyl]-3-yl)pentanal (5)

To a solution of 5-(4′-((tetrahydro-2H-pyran-2-yl)oxy)-[1,1′-biphenyl]-3-yl)pentan-1-ol (4, 0.470 g, 1.0 eq, 1.38 mmol) in dichloromethane (5 mL) at 0° C. was added pyridinium chloro chromate (0.446 g, 1.5 eq, 2.07 mmol) and reaction mixture stirred at room temperature for 4 h. After completion, reaction mixture was filtered over celite pad and washed with ether. Filtrate obtained was concentrated under reduced pressure and crude obtained was purified by combiflash chromatography using silica gel column and 10 to 20% ethyl acetate in hexane as eluents. Desired fractions were concentrated under reduced pressure to obtain 5-(4′-((tetrahydro-2H-pyran-2-yl)oxy)-[1,1′-biphenyl]-3-yl)pentanal (5) as colorless sticky gum. Yield: 0.290 g, 62.07%; LC-MS m/z 339.22 [M−1]⁻

### Synthesis of 2-((3′-(hex-5-yn-1-yl)-[1,1′-biphenyl]-4-yl)oxy)tetrahydro-2H-pyran (6)

To a solution of 5-(4′-((tetrahydro-2H-pyran-2-yl)oxy)-[1,1′-biphenyl]-3-yl)pentanal (5, 0.29 g, 1.0 eq, 0.857 mmol) in methanol (15.0 mL) was added potassium carbonate (0.296 g, 2.5 eq, 2.14 mmol) and 10% dimethyl (1-diazo-2-oxopropyl)phosphonate in acetonitrile (5a, 3.29 mL, 2.0 eq, 1.71 mmol) at 0° C. and reaction mixture was stirred at room temperature for 3 h. Reaction mixture quenched by addition of cold water and extracted with ethyl acetate. Ethyl acetate layer dried over anhydrous sodium sulphate and concentrated under reduced pressure to get crude compound. Crude compound obtained was purified by flash column chromatography using silica gel column and with 0 to 20% ethyl acetate in hexane as eluents. The desired fractions were concentrated under reduced pressure to get 2-((3′-(hex-5-yn-1-yl)-[1,1′-biphenyl]-4-yl)oxy)tetrahydro-2H-pyran (6) as colorless sticky gum. Yield: 0.25 g, 87%; LC-MS m/z 353.25 [M+18]⁺

### Synthesis of 3′-(hex-5-yn-1-yl)-[1,1′-biphenyl]-4-ol (7)

To a solution of 2-((3′-(hex-5-yn-1-yl)-[1,1′-biphenyl]-4-yl)oxy)tetrahydro-2H-pyran (6, 0.25 g, 0.747 mmol) in Methanol (3.00 mL) at 0° C., was added p-toluene sulphonic acid (0.014 g, 0.1 eq, 0.074 mmol) and reaction mixture stirred at room temperature for 2 h. Reaction mixture concentrated under reduced pressure and partitioned in between dichloromethane and aqueous sodium bicarbonate solution. Dichloromethane layer separated washed with brine solution, dried over anhydrous sodium sulphate and concentrated under reduced pressure to get crude product. Crude product obtained was purified by combiflash column chromatography using silica gel column and 5 to 15% Ethyl acetate in hexane as eluents. Desired fractions were concentrated under reduced pressure to afford 3′-(hex-5-yn-1-yl)-[1,1′-biphenyl]-4-ol (7) as colorless sticky gum. Yield: 0.16 g, 85%; LC-MS m/z 249.12 [M−1]⁻

### Synthesis of (2R,3R,4S,5S,6R)-2-(2-(diethoxyphosphoryl)ethyl)-6-((3′-(hex-5-yn-1-yl)-[1,1′-biphenyl]-4-yl)oxy)tetrahydro-2H-pyran-3,4,5-triyl triacetate (8)

To a stirred solution of (2R,3S,4S,5R,6R)-6-(2-(diethoxyphosphoryl)ethyl)tetrahydro-2H-pyran-2,3,4,5-tetrayl tetraacetate (7a, 1.45 g, 1.5 eq., 3.00 mmol) and 3′-(hex-5-yn-1-yl)-[1,1′-biphenyl]-4-ol (7, 0.50 g, 1.0 eq, 2.00 mmol)) in dry dichloromethane (20 mL) was added activated molecular sieves (100 mg) and reaction mixture stirred at room temperature for 15 mins. Reaction mixture cooled to 0° C. and borontrifluoride etherate (1.48 mL, 6 eq, 12.0 mmol) was slowly added to reaction mixture and reaction mixture allowed to come at room temperature and stirred at 50° C. for 16 h. Reaction mixture partitioned in between dichloromethane and aqueous sodium bicarbonate solution. Dichloromethane layer separated and washed with brine solution, dried over anhydrous sodium sulfate, filtered and concentrated under reduced pressure to get crude product. Crude product obtained was purified by combiflash column chromatography using silica gel column and 30 to 50% Ethyl acetate in dichloromethane as eluents. Desired fractions were concentrated under reduced pressure to afford (2R,3R,4S,5S,6R)-2-(2-(diethoxyphosphoryl)ethyl)-6-((3′-(hex-5-yn-1-yl)-[1,1′-biphenyl]-4-yl)oxy)tetrahydro-2H-pyran-3,4,5-triyl triacetate (8) as pale yellow sticky gum. Yield: 0.70 g, 52%; LC-MS m/z 673.39 [M+1]⁺

### Synthesis of (2-((2R,3R,4S,5S,6R)-3,4,5-triacetoxy-6-((3′-(hex-5-yn-1-yl)-[1,1′-biphenyl]-4-yl)oxy)tetrahydro-2H-pyran-2-yl)ethyl)phosphonic acid (9)

To the stirred solution of (2R,3R,4S,5S,6R)-2-(2-(diethoxyphosphoryl)ethyl)-6-((3′-(hex-5-yn-1-yl)-[1,1′-biphenyl]-4-yl)oxy)tetrahydro-2H-pyran-3,4,5-triyl triacetate (8, 0.720 g, 1.0 eq, 1.07 mmol) in dichloromethane (30.00 mL) at 0° C., Pyridine (1.30 mL, 15 eq, 16.1 mmol) and Bromotrimethylsilane (1.39 mL, 10 eq, 10.7 mmol) were added and reaction mixture was stirred at room temperature for 3 h. After completion reaction mixture was diluted with water and extracted with dichloromethane. Dichloromethane layer obtained was dried over anhydrous sodium sulphate and concentrated under reduced pressure to afford (2-((2R,3R,4S,5S,6R)-3,4,5-triacetoxy-6-((3′-(hex-5-yn-1-yl)-[1,1′-biphenyl]-4-yl)oxy)tetrahydro-2H-pyran-2-yl)ethyl)phosphonic acid (9) as pale yellow sticky gum. Yield: 0.60 g, 90.92%; LC-MS m/z 615.11 [M−1]⁻

### Synthesis of (2-((2R,3S,4S,5S,6R)-6-((3′-(hex-5-yn-1-yl)-[1,1′-biphenyl]-4-yl)oxy)-3,4,5-trihydroxytetrahydro-2H-pyran-2-yl)ethyl)phosphonic acid (Cpd. No. 63A)

To a solution of (2-((2R,3R,4S,5S,6R)-3,4,5-triacetoxy-6-((3′-(hex-5-yn-1-yl)-[1,1′-biphenyl]-4-yl)oxy)tetrahydro-2H-pyran-2-yl)ethyl)phosphonic acid (9, 0.630 g, 1 eq, 1.02 mmol) in Methanol (10.0 mL) at 0° C. was added Sodium methoxide solution (25%, 0.66 mL, 3 eq, 3.06 mmol) and reaction mixture stirred at room temperature for 3 h. LCMS showed formation of desired compound. Reaction mixture cooled down and neutralized Dowex 50W X8 hydrogen form up to pH 6 and filtered over sintered flask. Filtrate obtained was concentrated under reduced pressure to get crude product. Crude product obtained was purified by reverse phase preparative HPLC using 38-53% acetonitrile in water with 0.1% trifluoro acetic acid (0 to 10 minutes). Desired fractions were combined and lyophilized to afford (2-((2R,3S,4S,5S,6R)-6-((3′-(hex-5-yn-1-yl)-[1,1′-biphenyl]-4-yl)oxy)-3,4,5-trihydroxytetrahydro-2H-pyran-2-yl)ethyl)phosphonic acid (Compound 63A) as off white solid. Yield: 0.246 g, 49.09%; LC-MS m/z 491.13 [M+1]^{+ 1}H-NMR (400 MHz, DMSO-d₆) δ 7.60 (d, J=8.8 Hz, 2H), 7.44-7.41 (m, 2H), 7.33 (t, J=7.60 Hz, 1H), 7.15-7.10 (m, 3H), 5.43 (s, 1H), 5.07-4.78 (bm, 3H), 3.84 (s, 1H), 3.67-3.65 (m, 1H), 3.38-3.28 (m, 2H), 2.74 (bs, 1H), 2.64 (t, J=7.20 Hz, 2H), 2.21-2.17 (m, 2H), 1.97-1.94 (m, 1H), 1.71-1.65 (m, 2H), 1.58-1.45 (m, 4H), 1.22-1.12 (m, 1H).

### Example 64: Synthesis of (1,1-difluoro-2-((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)thioureido)phenoxy)tetrahydro-2H-pyran-2-yl)ethyl)phosphonic acid (Cpd. No. I-64)

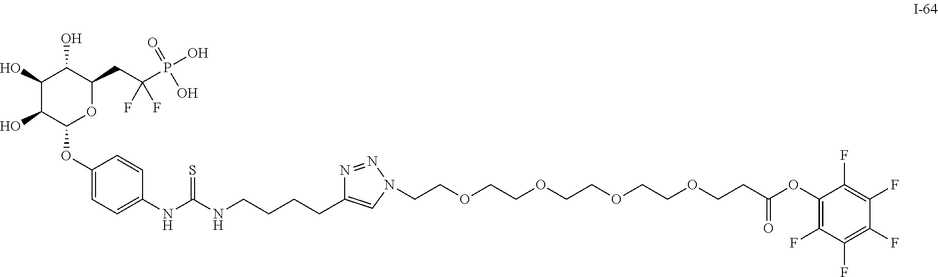 [SMILES: O=C(CCOCCOCCOCCOCCn1cc(CCCCNC(=S)Nc2ccc(O[C@H]3O[C@H](CC(F)(F)P(=O)(O)O)[C@@H](O)[C@H](O)[C@@H]3O)cc2)nn1)Oc1c(F)c(F)c(F)c(F)c1F]

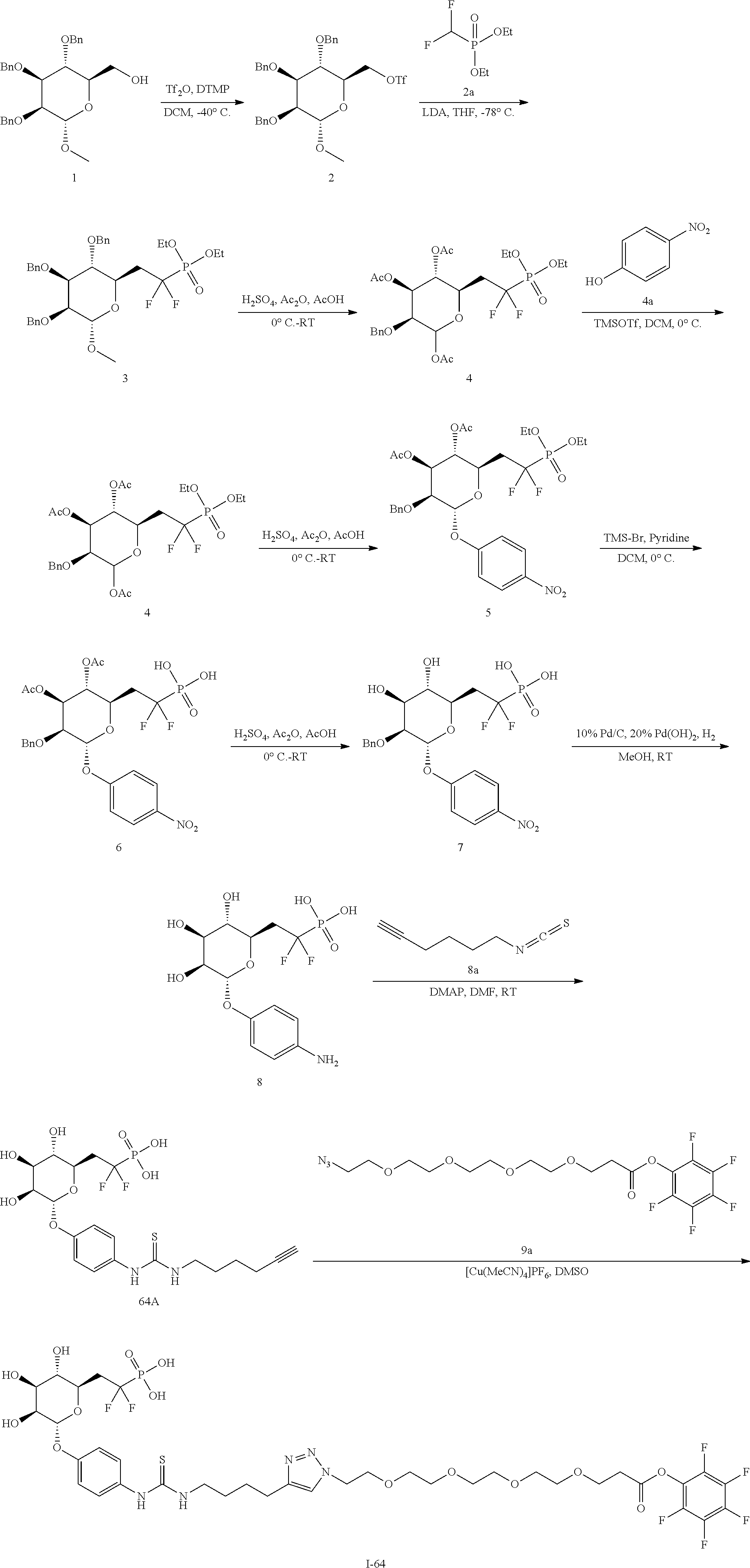

### Synthesis of ((2R,3R,4S,5S,6S)-3,4,5-tris(benzyloxy)-6-methoxytetrahydro-2H-pyran-2-yl)methyl trifluoromethanesulfonate (2)

To the stirred solution of ((2R,3R,4S,5S,6S)-3,4,5-tris(benzyloxy)-6-methoxytetrahydro-2H-pyran-2-yl)methanol (1, 1.0 eq, 5.0 g, 10.8 mmol) in dichloromethane (50 mL), 2,6-di-tert-butyl-4-methylpyridine (1.8 eq, 3.32 g, 16.1 mmol) and trifluoromethanesulfonic anhydride (1.5 eq, 2.35 mL, 14.0 mmol) were added at −40° C. and reaction mixture was stirred at same temperature for 1 h. The progress of reaction was monitored by TLC. After completion, the reaction mixture was concentrated under reduced pressure to get crude product. The crude was immediately purified by flash column chromatography using 15-50% ethyl acetate in hexane to afford ((2R,3R,4S,5S,6S)-3,4,5-tris(benzyloxy)-6-methoxytetrahydro-2H-pyran-2-yl)methyl trifluoromethanesulfonate (2) as a pale yellow gel and immediately used for next reaction.

### Synthesis diethyl (1,1-difluoro-2-((2R,3R,4S,5S,6S)-3,4,5-tris(benzyloxy)-6-methoxytetrahydro-2H-pyran-2-yl)ethyl)phosphonate (3)

To a stirred solution of diethyl (difluoromethyl)phosphonate (2a, 4.0 eq, 5.30 g, 28.2 mmol) and [bis(dimethylamino)phosphoryl]dimethylamine (4.0 eq, 5.05 g, 28.2 mmol) in tetrahydrofuran (25 mL), Lithium di-isopropyl amide (LDA) 2 M in tetrahydrofuran (4.0 eq, 14.1 mL, 28.2 mmol) was added drop wise at −78° C. and stirred for 30 min at same temperature, Then a solution of ((2R,3R,4S,5S,6S)-3,4,5-tris(benzyloxy)-6-methoxytetrahydro-2H-pyran-2-yl)methyl trifluoromethanesulfonate (2, 1.0 eq, 4.20 g, 7.04 mmol) in tetrahydrofuran (25 mL) was added dropwise. The reaction mixture was stirred at −78° C. for 1 h. The progress of reaction was monitored by TLC. After completion, reaction mixture was quenched with saturated ammonium chloride solution, and extracted with ethyl acetate. The organic layer was dried over anhydrous sodium sulphate, filtered and concentrated under reduced pressure to get crude product. The crude product was purified by flash column chromatography using eluting from silica gel with 15-50% ethyl acetate in hexane to afford diethyl (1,1-difluoro-2-((2R,3R,4S,5S,6S)-3,4,5-tris(benzyloxy) methoxytetrahydro-2H-pyran-2-yl)ethyl)phosphonate (3) as brown oil. Yield: 2.40 g, (49%) LCMS m/z 655.3 [M+18]⁺.

### Synthesis (3S,4S,5R,6R)-3-(benzyloxy)-6-(2-(diethoxyphosphoryl)-2,2-difluoroethyl)tetrahydro-2H-pyran-2,4,5-triyl triacetate (4)

To a stirred solution of diethyl {1,1-difluoro-2-[(2R,3R,4S,5S,6S)-3,4,5-tris(benzyloxy)-6-methoxyoxan-2-yl]ethyl}phosphonate (1.0 eq, 7.0 g, 11.0 mmol) in acetic anhydride (80.0 eq, 83.4 mL, 882 mmol) and acetic acid (132.0 eq, 83.3 mL, 1.46 mol). Sulfuric acid (6.5 eq, 3.82 mL, 71.7 mmol) was added at 0° C. and reaction mixture was stirred at room temperature for 16 h. The progress of reaction was monitored by TLC. After the completion, reaction mixture was concentrated under reduced pressure to get a residue. The residue was diluted with water and extracted with ethyl acetate. The organic layer was washed with saturated sodium bicarbonate solution, dried over anhydrous sodium sulphate, filtered and concentrated to get crude product. The crude was purified by flash column chromatography using 30-50% ethylacetate in hexane to afford (3S,4S,5R,6R)-5-(benzyloxy)-6-(2-(diethoxyphosphoryl)-2,2-difluoroethyl)tetrahydro-2H-pyran-2,3,4-triyl triacetate (4) as colorless syrup. Yield: 3.20 g, (51%); LCMS m/z 566.3 [M+1]⁺.

### Synthesis of (2R,3R,4S,5S,6R)-5-(benzyloxy)-2-(2-(diethoxyphosphoryl)-2,2-difluoroethyl)-6-(4-nitrophenoxy)tetrahydro-2H-pyran-3,4-diyl diacetate (5)

To the stirred solution of (3S,4S,5R,6R)-3-(benzyloxy)-6-(2-(diethoxyphosphoryl)-2,2-difluoroethyl)tetrahydro-2H-pyran-2,4,5-triyl triacetate (4, 1.0 eq, 3.20 g, 5.65 mmol) in dichloromethane (40 mL), 4-nitrophenol (4a, 3.0 eq, 2.36 g, 16.9 mmol) was added followed by trimethylsilyl trifluoromethanesulfonate (1.0 eq, 1.03 mL, 5.65 mmol) and reaction mixture was stirred at 0° C. for 4 h. The progress of reaction was monitored by TLC. After the completion of reaction, mixture was quenched with ice water and extracted with dichloromethane. The organic layer was dried over anhydrous sodium sulfate, filtered and concentrated to get crude. The crude was purified by flash column chromatography using 30-80% ethyl acetate in hexane to afford (2R,3S,4S,5R,6R)-5-(benzyloxy)-6-(2-(diethoxyphosphoryl)-2,2-difluoroethyl)-2-(4-nitrophenoxy)tetrahydro-2H-pyran-3,4-diyl diacetate (5) as brown syrup. Yield: 2.45 g, (67.1%); LCMS m/z 663.20 [M+18]⁺.

### Synthesis of (2-((2R,3R,4S,5S,6R)-3,4-diacetoxy-5-(benzyloxy)-6-(4-nitrophenoxy)tetrahydro-2H-pyran-2-yl)-1,1-difluoroethyl)phosphonic acid (6)

To the stirred solution of(2R,3S,4S,5R,6R)-5-(benzyloxy)-6-(2-(diethoxyphosphoryl)-2,2-difluoroethyl)-2-(4-nitrophenoxy)tetrahydro-2H-pyran-3,4-diyl diacetate (5, 1.0 eq, 1.00 g, 1.55 mmol) in dichloromethane (25 mL), pyridine (10.0 eq, 1.25 mL 15.5 mmol) followed by bromotrimethylsilane (10.0 eq, 2.0 mL, 15.5 mmol) was added at 0° C. and reaction mixture was stirred under for 16 h. The reaction mixture was monitored by LC-MS. After the completion of reaction, reaction mixture was quenched with ice water and extracted with dichloromethane. The organic layer was dried over anhydrous sodium sulfate, filtered and concentrated to get crude. The crude was triturated with diethyl ether and dried to get (2-((2R,3R,4S,5S,6R)-4,5-diacetoxy-3-(benzyloxy)-6-(4-nitrophenoxy)tetrahydro-2H-pyran-2-yl)-1,1-difluoroethyl)phosphonic (6) acid as off white solid. Yield: 0.83 g, (90%); LCMS m/z 588.2 [M−1]⁻.

### Synthesis of (2-((2R,3S,4S,5S,6R)-5-(benzyloxy)-3,4-dihydroxy-6-(4-nitrophenoxy)tetrahydro-2H-pyran-2-yl)-1,1-difluoroethyl)phosphonic acid (7)

To a stirred solution of (2-((2R,3R,4S,5S,6R)-4,5-diacetoxy-3-(benzyloxy)-6-(4-nitrophenoxy)tetrahydro-2H-pyran-2-yl)-1,1-difluoroethyl)phosphonic acid (6. 1.0 eq, 1.10 g, 1.87 mmol) dissolved in methanol (30 mL) and dichloromethane (10 mL) at 0° C., sodium methoxide 25% w/v in methanol (10.0 eq, 1.07 mL, 18.7 mmol) was added drop-wise. The reaction mixture was stirred at room temperature. After 3 h, the reaction mixture was neutralized with Dowex-50 hydrogen form (up to pH 7), filtered and filtrate was concentrated under reduced pressure to afford crude of (2-((2R,3S,4R,5S,6R)-3-(benzyloxy)-4,5-dihydroxy-6-(4-nitrophenoxy)tetrahydro-2H-pyran-2-yl)-1,1-difluoroethyl)phosphonic acid (7) as off white solid Yield: 0.618 g, (66%); LCMS m/z 504.13 [M−1]⁻

### Synthesis of (2-((2R,3S,4S,5S,6R)-6-(4-aminophenoxy)-3,4,5-trihydroxytetrahydro-2H-pyran-2-yl)-1,1-difluoroethyl)phosphonic acid (8)

To a stirred solution of {2-[(2R,3S,4R,5S,6R)-3-(benzyloxy)-4,5-dihydroxy-6-(4-nitrophenoxy)oxan-2-yl]-1,1-difluoroethyl}phosphonic acid (7, 1.0 eq, 0.55 g, 1.10 mmol) in methanol (10 mL), 10% Palladium on carbon (0.27 g) and 20% Pd(OH)₂ (0.27 g) were added and purged with hydrogen gas and stirred under hydrogen atmosphere for 5 h at room temperature. Then reaction mixture was filtered through a syringe filter (NY 0.45 μm). The filtrate was evaporated under reduced pressure to get crude of {2-[(2R,3S,4S,5S,6R)-6-(4-aminophenoxy)-3,4,5-trihydroxyoxan-2-yl]-1,1-difluoroethyl}phosphonic acid (8). The crude product was directly used for the next reaction without further purification. Yield: 0.31 g, (40.8%); LCMS m/z 386.1 [M+1]⁺

### Synthesis of (1,1-difluoro-2-((2R,3S,4S,5S,6R)-6-(4-(3-(hex-5-yn-1-yl)thioureido)phenoxy)-3,4,5-trihydroxytetrahydro-2H-pyran-2-yl)ethyl)phosphonic acid (Cpd. No. 64A)

To a stirred solution of {2-[(2R,3S,4S,5S,6R)-6-(4-aminophenoxy)-3,4,5-trihydroxyoxan-2-yl]-1,1-difluoroethyl}phosphonic acid (8, 1.0 eq, 0.31 g, 0.815 mmol) and N,N-dimethylpyridin-4-amine (4.0 eq, 0.39 g, 3.26 mmol) in N,N-dimethylformamide (10 mL) at 0° C. was added a solution of 6-isothiocyanatohex-1-yne (8a, 3.0 eq, 0.34 g, 2.45 mmol) in N,N-dimethyl formamide (2 mL). The reaction mixture was then stirred at room temperature for 12 h. The reaction mixture was concentrated under reduced pressure to get crude. The crude was purified by prep- HPLC (10-30% Aceonitrile in water with 0.1% TFA). Fractions containing the desired product were combined and lyophilized to dryness to afford {1,1-difluoro-2-[(2R,3S,4S,5S,6R)-6-(4-{[(hex-5-yn-1-yl)carbamothioyl]amino}phenoxy)-3,4,5-trihydroxyoxan-2-yl]ethyl}phosphonic acid as white solid. (64A) as a white solid. Yield: 0.059 g, 13.8%; LCMS m/z 523.1 [M−1]⁻;

### Synthesis of 2-((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)thioureido)phenoxy)tetrahydro-2H-pyran-2-yl)ethane-1-sulfonic acid (I-64)

To a solution of (1,1-difluoro-2-((2R,3S,4S,5S,6R)-6-(4-(3-(hex-5-yn-1-yl)thioureido)phenoxy)-3,4,5-trihydroxytetrahydro-2H-pyran-2-yl)ethyl)phosphonic acid (64A, 1.0 eq, 0.055 g, 0.090 mmol) in dimethylsulfoxide (1.5 mL), perfluorophenyl 1-azido-3,6,9,12-tetraoxapentadecan-15-oate (9a, 1.0 eq, 0.041 g, 0.090 mmol) was added and stirred for 5 minutes. Then, tetrakis(acetonitrile)copper(I) hexafluorophosphate (2.8 eq, 0.093 g, 0.253 mmol) was added and reaction mixture was stirred at room temperature for 20 min. After completion, reaction mixture was diluted with acetonitrile and purified by prep HPLC (50-65% acetonitrile in water with 0.1% TFA). Fractions containing the desired product were combined and lyophilized to dryness to afford (1,1-difluoro-2-((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)thioureido)phenoxy)tetrahydro-2H-pyran-2-yl)ethyl)phosphonic acid (I-64) as white solid. Yield: 0.032 g, 33%; LCMS m/z 982.4 [M+1]⁺; ¹H NMR (400 MHz, DMSO-d6) δ 9.26 (s, 1H), 7.81 (s, 1H), 7.56 (s, 1H), 7.23 (d, J=8.8 Hz, 2H), 7.00 (d, J=8.8 Hz, 2H), 5.20 (s, 1H), 5.06 (s, 1H), 4.82 (s, 1H), 4.45 (t, J=10.0 Hz, 2H), 3.87-3.74 (m, 7H), 3.67 (t, J=9.6 Hz, 1H), 3.54-3.51 (m, 3H), 3.49-3.30 (m, 13H), 3.01 (t, J=5.6 Hz, 2H), 2.66-2.50 (m, 4H), 2.07-1.95 (m, 1H), 1.63-1.57 (m, 4H).

### Example 65: Synthesis of 2-((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)thioureido)phenoxy)tetrahydro-2H-pyran-2-yl)ethane-1-sulfonic acid (Cpd. No. I-65)

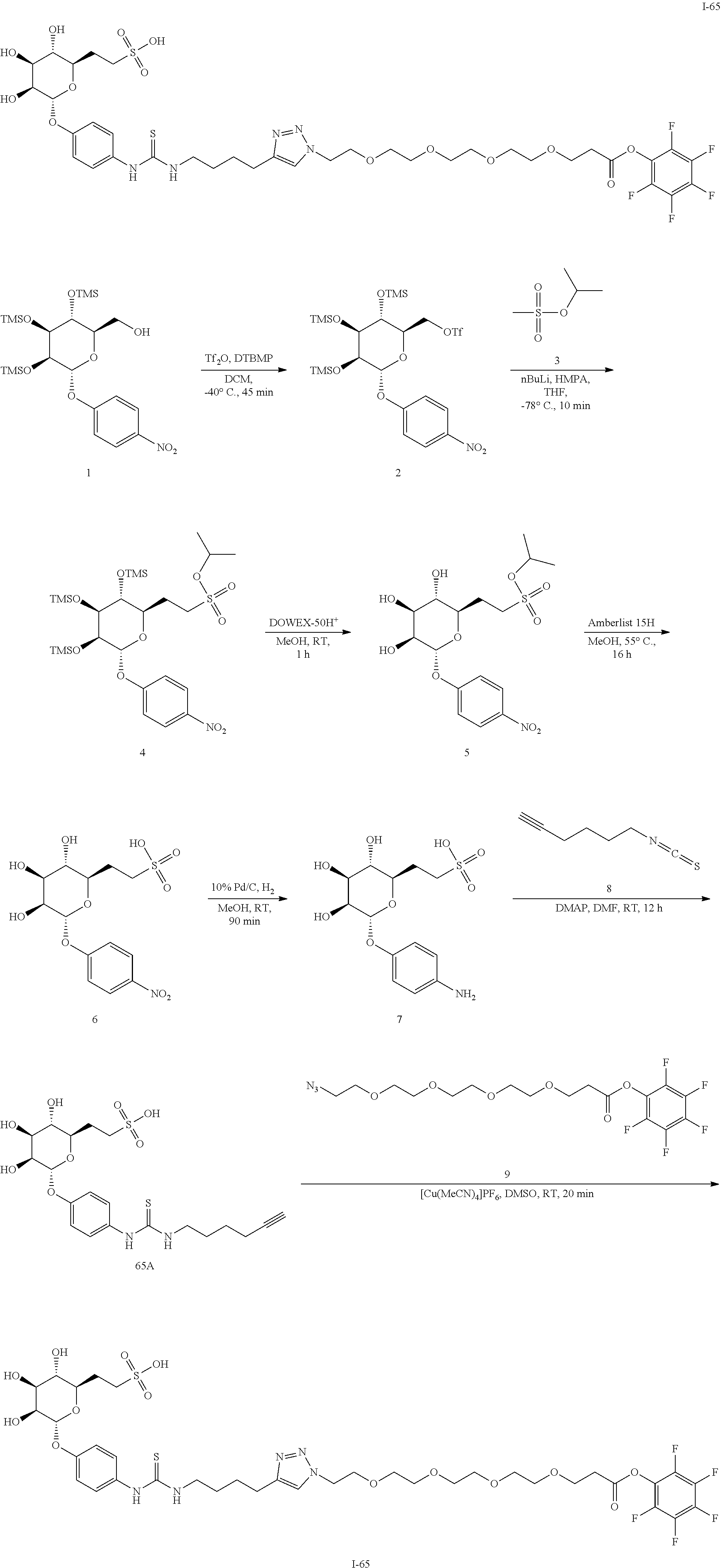

### Synthesis of ((2R,3R,4S,5S,6R)-6-(4-nitrophenoxy)-3,4,5-tris((trimethylsilyl)oxy)tetrahydro-2H-pyran-2-yl)methyl trifluoromethanesulfonate (2)

To a stirred solution of [(2R,3R,4S,5S,6R)-6-(4-nitrophenoxy)-3,4,5-tris[(trimethylsilyl)oxy]oxan-2-yl]methanol (1, 4.0 g, 7.73 mmol) and 2,6-di-tert-butyl methylpyridine (3.17 g, 15.45 mmol) in dichloromethane (40.0 mL) was added trifluoromethanesulfonic anhydride (1.69 mL, 10.04 mmol) dropwise at −40° C. under a nitrogen atmosphere. After stirring for 1 h at −40° C., TLC showed full conversion. The volatiles were then evaporated and the crude ((2R,3R,4S,5S,6R)-6-(4-nitrophenoxy)-3,4,5-tris((trimethylsilyl)oxy)tetrahydro-2H-pyran-2-yl)methyl trifluoromethanesulfonate (2) was directly used for the next reaction.

### Synthesis of isopropyl 2-((2R,3R,4S,5S,6R)-6-(4-nitrophenoxy)-3,4,5-tris((trimethylsilyl)oxy)tetrahydro-2H-pyran-2-yl)ethane-1-sulfonate (4)

n-BuLi (12.3 mL, 30.8 mmol, 2.5 M solution in hexane) was added dropwise to a stirred solution of isopropyl methylsulfonate (3, 3.75 mL, 30.8 mmol) and [bis(dimethylamino)phosphoryl]dimethylamine (6.69 mL, 38.5 mmol) in dry tetrahydrofuran (60.0 mL) at −78° C. under nitrogen atmosphere. After 30 min, a pre-cooled solution of [(2R,3R,4S,5S,6R)-6-(4-nitrophenoxy)-3,4, 5-tris[(trimethylsilyl)oxy]oxan-2-yl]methyl trifluoromethanesulfonate (2, 5.0 g, 7.69 mmol) in dry tetrahydrofuran (40.0 mL) was added to the reaction mixture. After 10 min, the reaction mixture was quenched with aq. ammonium chloride solution. The reaction mixture was extracted twice with ethyl acetate (50.0 mL) and washed with saturated aq. sodium bicarbonate solution. Organic fractions were collected and then dried over anhydrous sodium sulfate and filtered. The filtrate was evaporated under vacuum. The crude mass was purified by silica gel column chromatography (using 15% ethyl acetate in hexane) to afford propan-2-yl 2-[(2R,3R,4S,5S,6R)-6-(4-nitrophenoxy)-3,4,5-tris[(trimethylsilyl)oxy]oxan-2-yl]ethane-1-sulfonate (4) as yellowish solid. Yield: 2.4 g, 49%; LCMS m/z 655.3 [M+18]⁺.

### Synthesis of isopropyl 2-((2R,3S,4S,5S,6R)-3,4,5-trihydroxy-6-(4-nitrophenoxy)tetrahydro-2H-pyran-2-yl)ethane-1-sulfonate (5)

To a stirred solution of propan-2-yl 2-[(2R,3R,4S,5S,6R)-6-(4-nitrophenoxy)-3,4,5-tris[(trimethylsilyl)oxy]oxan-2-yl]ethane-1-sulfonate (4, 1.7 g, 2.66 mmol) in methanol (80 mL) was added DOWEX-50H (10 g). After stirring for 1 h at room temperature, the resin was filtered off, washed with methanol, and the collected methanol portion was evaporated under vacuum. The crude reaction mass was then purified by silica gel column chromatography (using 10% methanol in dichloromethane), gave propan-2-yl 2-[(2R,3S,4S,5S,6R)-3,4,5-trihydroxy-6-(4-nitrophenoxy)oxan-2-yl]ethane-1-sulfonate (5) as white foam. Yield: 0.845 g, 75%; LCMS m/z 420.1 [M−1]⁻

### Synthesis of 2-((2R,3S,4S,5S,6R)-3,4,5-trihydroxy-6-(4-nitrophenoxy)tetrahydro-2H-pyran-2-yl)ethane-1-sulfonic acid (6)

To a stirred solution of propan-2-yl 2-[(2R,3S,4S,5S,6R)-3,4,5-trihydroxy-6-(4-nitrophenoxy)oxan-2-yl]ethane-1-sulfonate (5, 1.15 g, 2.73 mmol) in methanol (60 ml) was added Amberlist-15H (20 g) and heated at 55° C. for 16 h. The resin was then filtered off, washed with methanol, and the collected methanol portion was evaporated under vacuum. The crude product was purified by reverse phase column chromatography (eluting from a C18 column with 1-2% acetonitrile in water). The fractions containing the desired product were collected and lyophilized to provide 2-[(2R,3S,4S,5S,6R)-3,4,5-trihydroxy-6-(4-nitrophenoxy)oxan-2-yl]ethane-1-sulfonic acid (6) as white solid. Yield: 0.776 g, 75%; LCMS m/z 378.0 [M−1]⁻

### Synthesis of 2-((2R,3S,4S,5S,6R)-6-(4-aminophenoxy)-3,4,5-trihydroxytetrahydro-2H-pyran-2-yl)ethane-1-sulfonic acid (7)

To a stirred solution of 2-[(2R,3S,4S,5S,6R)-3,4,5-trihydroxy-6-(4-nitrophenoxy)oxan-2-yl]ethane-1-sulfonic acid (6, 0.103 g, 0.272 mmol) in methanol-water (10 ml, 9:1, v/v) was added 10% Pd/C (200.0 mg) and then purged with hydrogen gas and kept under hydrogen atmosphere for 90 min at room temperature. Then reaction mixture was filtered through NY 0.45 μm filter. The volatiles were then evaporated under reduced pressure to yield 2-((2R,3S,4S,5S,6R)-6-(4-aminophenoxy)-3,4,5-trihydroxytetrahydro-2H-pyran-2-yl)ethane-1-sulfonic acid (7) as white foam. Yield: 0.092 g, 96%; LCMS m/z 350.0 [M+1]⁺

### Synthesis of 2-((2R,3S,4S,5S,6R)-6-(4-(3-(hex-5-yn-1-yl)thioureido)phenoxy)-3,4,5-trihydroxytetrahydro-2H-pyran-2-yl)ethane-1-sulfonic acid (65A)

To a stirred solution of 2-[(2R,3S,4S,5S,6R)-6-(4-aminophenoxy)-3,4,5-trihydroxyoxan-2-yl]ethane-1-sulfonic acid (7, 0.179 g, 0.512.0 mmol) and N,N-dimethylpyridin-4-amine (0.188 g, 1.54 mmol) in N,N-dimethylformamide (10 mL) at 0° C. was added a solution of 6-isothiocyanatohex-1-yne (8, 0.214 mg, 1.54 mmol) in N,N-dimethylformamide (2 mL). The reaction mixture was then stirred at room temperature for 12 h. After completion, reaction mixture was diluted with acetonitrile and purified by prep HPLC (15-47% acetonitrile in water with 0.1% TFA). Fractions containing the desired product were combined and lyophilized to dryness to afford 2-((2R,3S,4S,5S,6R)-6-(4-(3-(hex-5-yn-1-yl)thioureido)phenoxy)-3,4,5-trihydroxytetrahydro-2H-pyran-2-yl)ethane-1-sulfonic acid (Cpd. No. 65A) as a white solid. Yield: 0.080 g, 32%; LCMS m/z 489.2 [M+1]⁺; ¹H NMR (400 MHz, D₂O) 7.26-7.23 (m, 2H), 7.20-7.17 (m, 2H), 5.63 (s, 1H), 4.18 (s, 1H), 4.01 (d, J=9.6 Hz, 1H), 3.68. (t, J=9.6 Hz, 1H), 3.62-3.55 (m, 3H), 2.95-2.88 (m, 1H), 2.66-2.59 (m, 1H), 2.39-2.25 (m, 4H), 1.89-1.80 (m, 1H), 1.68 (brs, 2H), 1.53 (brs, 2H).

### Synthesis of 2-((2R,3S,4S,5S,6R)-3,4,5-trihydroxy-6-(4-(3-(4-(1-(15-oxo (perfluorophenoxy)-3,6,9,12-tetraoxapentadecyl)-1H-1,2,3-triazol yl)butyl)thioureido)phenoxy)tetrahydro-2H-pyran-2-yl)ethane-1-sulfonic acid (Compound I-65)

To a stirred solution of 2-[(2R,3S,4S,5S,6R)-6-(4-{[(hex-5-yn yl)carbamothioyl]amino}phenoxy)-3,4,5-trihydroxyoxan-2-yl]ethane-1-sulfonic acid (65A 0.031 g, 0.063 mmol) in dimethylsulfoxide (0.5 mL) at 10° C. was added a solution of 2,3,4,5,6-pentafluorophenyl 1-azido-3,6,9,12-tetraoxapentadecan-15-oate (9, 0.029 g, 0.063 mmol) in dimethylsulfoxide (0.5 mL) and nitrogen gas was purged in reaction mixture for 1 minute. λ¹-copper(I) tetrakis(acetonitrile) hexafluoride λ⁻⁵-phosphanepentauide (0.066 g, 2.8 eq., 0.178 mmol) was added at 10° C. and reaction mixture was stirred at room temperature for 10 min. LCMS showed formation of desired compound. After completion, reaction mixture was diluted with acetonitrile and purified by prep. HPLC (30-70% acetonitrile in water with 0.1% TFA) to obtain 2,3,4,5,6-pentafluorophenyl 3-{2-[2-({20-[4-(2-{[(2R,3R,4R,5R,6R)-3-acetamido-4,5-dihydroxy-6-(hydroxymethyl)oxan-2-yl]oxy}ethyl)-1H-1,2,3-triazol-1-yl]-3,6,9,12,15,18-hexaoxaicosan-1-yl}carbamoyl)ethoxy]ethoxy}propanoate (1-65) as white solid. Yield: 20.0 mg, 33%; LCMS m/z 946.4 [M+1]⁺; ¹H NMR (400 MHz, D₂O) 7.90 (s, 1H), 7.20-7.18 (m, 4H), 5.63 (s, 1H), 4.60 (brs, 2H), 4.19 (s, 1H), 4.03-3.93 (m, 5H), 3.71-3.55 (m, 16H), 3.07 (brs, 2H), 2.92 (brs, 1H), 2.77 (s, 2H), 2.64-2.63 (1H), 2.25 (brs, 1H), 1.88 (brs, 1H), 1.74-1.59 (m, 4H).

### Example 66: Synthesis of 2-(((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)thioureido)phenoxy)tetrahydro-2H-pyran-2-yl)methyl)malonic acid (Cpd. No. I-66)

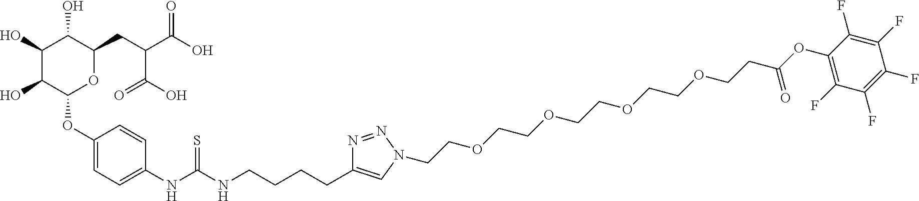 [SMILES: O=C(CCOCCOCCOCCOCCn1cc(CCCCNC(=S)Nc2ccc(O[C@H]3O[C@H](CC(C(=O)O)C(=O)O)[C@@H](O)[C@H](O)[C@@H]3O)cc2)nn1)Oc1c(F)c(F)c(F)c(F)c1F]

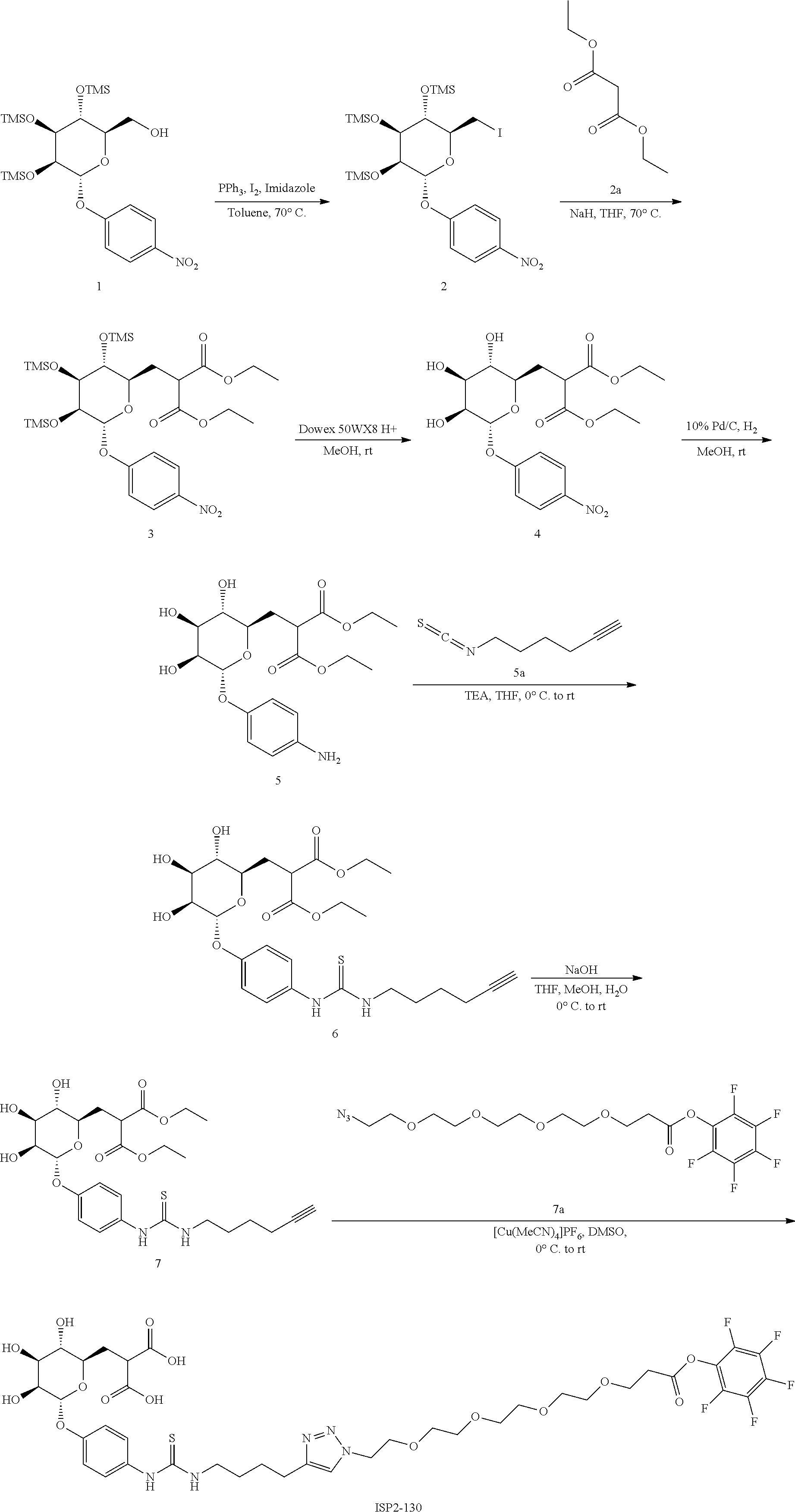

### Synthesis of (((2S,3R,4S,5S,6R)-2-(iodomethyl)-6-(4-nitrophenoxy)tetrahydro-2H-pyran-3,4,5-triyl)tris(oxy))tris(trimethylsilane) (2)

A solution of ((2R,3R,4S,5S,6R)-6-(4-nitrophenoxy)-3,4,5-tris((trimethylsilyl)oxy)tetrahydro-2H-pyran-2-yl)methanol (1, 1.00 g, 1.0 eq, 1.93 mmol), 1H-imidazole (0.394 g, 3 eq, 5.79 mmol), triphenyl phosphine (0.503 g, 1.0 eq, 1.93 mmol) and Iodine (0.61 g, 2.5 eq., 4.83 mmol) in toluene (15 mL), was heated to 70° C. and allowed to stir for another 12 h at this temperature. Reaction mixture was cooled down, diluted with ethyl acetate and quenched by addition of water. Ethyl acetate layer separated and aqueous layer re-extracted with ethyl acetate. Combined organic layer was dried over anhydrous sodium sulphate and evaporated under reduced pressure to get a crude residue which was purified by flash column chromatography using silica gel column and 0 to 3% ethyl acetate-hexane as eluents. Desired fractions were concentrated under reduced pressure to afford (((2S,3R,4S,5S,6R)-2-(iodomethyl)-6-(4-nitrophenoxy)tetrahydro-2H-pyran-3,4,5-triyl)tris(oxy))tris(trimethylsilane) (2) as off white solid. Yield: 590 mg, 49%; LC-MS m/z 628.0 [M+1]⁺.

### Synthesis of diethyl 2-(((2R,3R,4S,5S,6R)-6-(4-nitrophenoxy)-3,4,5-tris((trimethylsilyl)oxy)tetrahydro-2H-pyran-2-yl)methyl)malonate (3)

To a solution of diethylmalonate (1.99 g, 3 eq., 12.4 mmol) in dry tetrahydrofuran (20 mL) was added sodium hydride (0.497 g, 3 eq., 12.4 mmol) and stirred for 10 minutes. (((2S,3R,4S,5S,6R)-2-(iodomethyl)-6-(4-nitrophenoxy)tetrahydro-2H-pyran-3,4,5-triyl)tris(oxy))tris(trimethylsilane) (2, 2.60 g, 1.0 eq, 4.14 mmol) in dry tetrahydrofuran (10 mL) was added slowly to reaction mixture and reaction mixture stirred at 70° C. for 24 h. TLC and LCMS showed presence of starting material and formation of desired product. Reaction mixture quenched by addition of cold water and extracted with ethyl acetate. Ethyl acetate layer dried over anhydrous sodium sulphate and concentrated under reduced pressure to get crude product. Crude product obtained was purified by combiflash using silica gel column (40 g) and a gradient of 3 to 10% ethyl acetate in hexane as eluents to recover starting material (((2S,3R,4S,5S,6R)-2-(iodomethyl)-6-(4-nitrophenoxy)tetrahydro-2H-pyran-3,4,5-triyl)tris(oxy))tris(trimethylsilane) (2, 1.20 g) and afford the desired compound diethyl 2-(((2R,3R,4S,5S,6R)-6-(4-nitrophenoxy)-3,4,5-tris((trimethylsilyl)oxy)tetrahydro-2H-pyran-2-yl)methyl)malonate (3) as pale yellow sticky gum. Yield: 1.40 g, 51.2%; LC-MS m/z 658.2 [M−1]⁻.

### Synthesis of diethyl 2-(((2R,3S,4S,5S,6R)-3,4,5-trihydroxy-6-(4-nitrophenoxy)tetrahydro-2H-pyran-2-yl)methyl)malonate (4)

To a solution of diethyl 2-(((2R,3R,4S,5S,6R)-6-(4-nitrophenoxy)-3,4,5-tris((trimethylsilyl)oxy) tetrahydro-2H-pyran-2-yl) methyl) malonate (3, 1.90 g, 1.0 eq, 2.88 mmol) in methanol (20.0 mL) was added Dowex 50W X8 hydrogen form (0.10 g) and reaction mixture stirred at room temperature for 3 h. Reaction mixture filtered over sintered glass funnel and filtrate obtained was concentrated under reduced pressure to get crude product. The crude product was purified by combiflash column chromatography using silica gel column (12 g) and 4 to 5% methanol in dichloromethane as eluents to afford diethyl 2-(((2R,3S,4S,5S,6R)-3,4, 5-trihydroxy-6-(4-nitrophenoxy)tetrahydro-2H-pyran-2-yl)methyl)malonate (4) as pale yellow solid. Yield: 0.80 g, 62.6%; LC-MS 442.2 m/z [M−1]⁻.

### Synthesis of diethyl 2-(((2R,3S,4S,5S,6R)-6-(4-aminophenoxy)-3,4,5-trihydroxytetrahydro-2H-pyran-2-yl)methyl)malonate (5)

To a solution of afford diethyl 2-(((2R,3S,4S,5S,6R)-3,4,5-trihydroxy-6-(4-nitrophenoxy)tetrahydro-2H-pyran-2-yl)methyl)malonate (4, 0.80 g, 1.0 eq, 1.80 mmol) in methanol (15 mL) was added 10% Pd/C (0.20 g) and reaction mixture stirred at room temperature under hydrogen atmosphere for 3 h. TLC showed consumption of starting material. The reaction mixture was filtered over a celite pad to remove catalyst and the filtrate was concentrated under reduced pressure to get pure diethyl 2-(((2R,3S,4S,5S,6R)-6-(4-aminophenoxy)-3,4,5-trihydroxytetrahydro-2H-pyran-2-yl)methyl)malonate (5) as pale yellow solid. Yield: 0.62 g, 83.1%; LC-MS m/z 414.1 [M+1]⁺.

### Synthesis of diethyl 2-(((2R,3S,4S,5S,6R)-6-(4-(3-(hex-5-yn-1-yl)thioureido)phenoxy)-3,4,5-trihydroxytetrahydro-2H-pyran-2-yl)methyl)malonate (6)

To a solution of diethyl 2-(((2R,3S,4S,5S,6R)-6-(4-aminophenoxy)-3,4,5-trihydroxytetrahydro-2H-pyran-2-yl)methyl)malonate (5, 0.40 g, 1.0 eq, 0.968 mmol) in tetrahydrofuran (10.0 mL) at 0° C. was added triethylamine (0.337 mL, 2.5 eq, 2.42 mmol) and 6-isothiocyanatohex-1-yne (5a, 0.337 g, 2.5 eq, 2.42 mmol) dissolved in tetrahydrofuran (3 mL). Reaction mixture then stirred at room temperature for 16 h. Reaction mixture concentrated under reduced pressure and purified by combiflash column chromatography using silica gel column and eluting product in 5% methanol in dichloromethane as eluents. Desired fractions were concentrated under reduced pressure to afford diethyl 2-(((2R,3S,4S,5S,6R)-6-(4-(3-(hex-5-yn-1-yl)thioureido)phenoxy)-3,4,5-trihydroxytetrahydro-2H-pyran-2-yl)methyl)malonate (6) as pale yellow solid. Yield: 0.283 g, 50.2%; LC-MS m/z 553.3 (M+1)⁺

### Synthesis of 2-(((2R,3S,4S,5S,6R)-6-(4-(3-(hex-5-yn-1-yl)thioureido)phenoxy)-3,4,5-trihydroxytetrahydro-2H-pyran-2-yl)methyl)malonic acid (7)

To a solution of diethyl 2-(((2R,3S,4S,5S,6R)-6-(4-(3-(hex-5-yn-1-yl)thioureido)phenoxy)-3,4,5-trihydroxytetrahydro-2H-pyran-2-yl)methyl)malonate (6, 0.28 g, 1.0 eq, 0.512 mmol) in tetrahydrofuran (10.0 mL) and methanol (1.0 mL) at 0° C. was added a solution of NaOH (0.041 g, 2 eq, 1.02 mmol) in water (0.5 mL) and reaction mixture stirred at room temperature for 1 h. LCMS showed formation of desired compound. Reaction mixture was neutralized with 2N hydrochloric acid to pH 6 and reaction mixture was concentrated under reduced pressure to get crude product. Crude product obtained was purified by reverse phase preparative HPLC (20 to 30% acetonitrile in water with 0.1% trifluoroacetic acid). Fractions containing the desired product were combined and lyophilized to dryness to afford 22-(((2R,3S,4S,5S,6R)-6-(4-(3-(hex-5-yn-1-yl)thioureido)phenoxy)-3,4,5-trihydroxytetrahydro-2H-pyran-2-yl)methyl)malonic acid (7) as off white solid. Yield: 0.12 g, 47.9%; LC-MS m/z 497.2 (M+1)⁺

### Synthesis of 2-(((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)thioureido)phenoxy)tetrahydro-2H-pyran-2-yl)methyl)malonic acid (Cpd. No. I-66)

To a solution of 2-(((2R,3S,4S,5S,6R)-6-(4-(3-(hex-5-yn-1-yl)thioureido)phenoxy)-3,4,5-trihydroxytetrahydro-2H-pyran-2-yl)methyl)malonic acid (7, 0.020 g, 0.040 mmol) in dimethyl sulfoxide (0.80 mL) was added perfluorophenyl 1-azido-3,6,9,12-tetraoxapentadecan-15-oate (0.018 g, 0.040 mmol) and tetrakis(acetonitrile)copper(I) hexafluorophosphate (0.037 g, 0.1 mmol). The reaction mixture was stirred at room temperature for 10 minutes. Reaction mixture was purified directly by reverse phase preparative HPLC eluting the product with a gradient of 42 to 60% Acetonitrile in water with 0.1% trifluoroacetic acid buffer to afford 2-(((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol yl)butyl)thioureido)phenoxy)tetrahydro-2H-pyran-2-yl)methyl)malonic acid (Cpd. No. I-66) as pale yellow solid. Yield: 0.012 g, 31%; LC-MS m/z 954.3 [M+1]⁺; ¹H-NMR (400 MHz, DMSO-d6) δ 9.31 (bs, 1H), 7.81 (s, 1H), 7.44 (bs, 1H), 7.23-7.21 (m, 2H), 6.95 (d, J=8.8 Hz, 2H), 5.25 (s, 1H), 4.46-4.43 (m, 1H), 3.79-3.74 (m, 4H), 3.62-3.57 (m, 1H), 3.53-3.47 (m, 15H), 3.32 (bs, 5H), 3.23-3.19 (m, 1H), 3.03-3.00 (m, 2H), 2.66-2.60 (m, 2H), 2.36-2.32 (m, 1H), 1.71-1.56 (m, 6H).

### Example 67: (2-((2R,3S,4S,5S,6R)-6-((4-(3-(hex-5-yn-1-yl)ureido)phenyl)thio)-3,4,5-trihydroxytetrahydro-2H-pyran-2-yl)ethyl)phosphonic acid (Cpd. No. I-67)

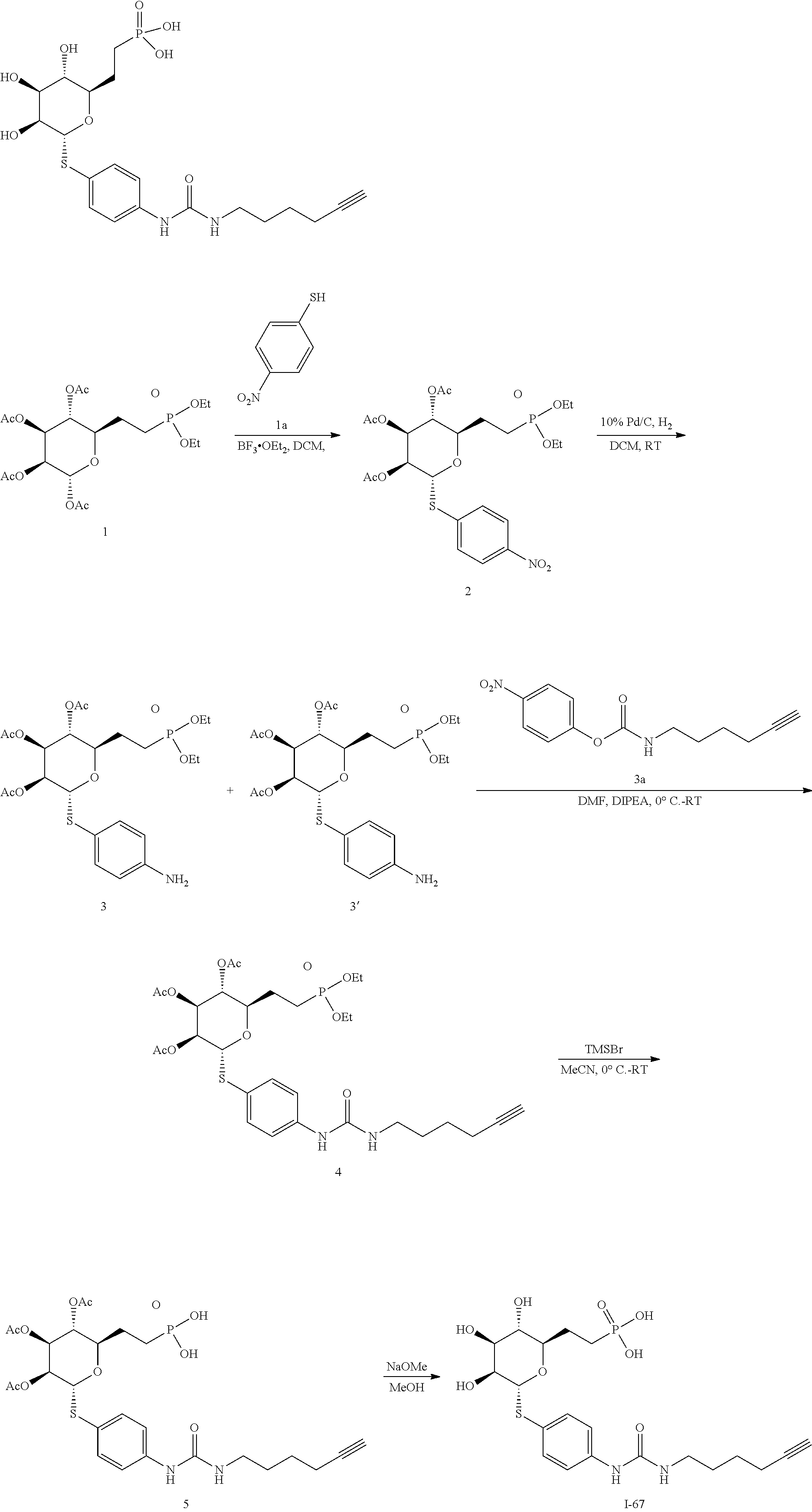

### Synthesis of (2R,3R,4S,5S,6R)-2-(2-(diethoxyphosphoryl)ethyl)-6-((4-nitrophenyl)thio)tetrahydro-2H-pyran-3,4,5-triyl triacetate (2)

To a stirred solution of (2R,3S,4S,5R,6R)-6-(2-(diethoxyphosphoryl)ethyl)tetrahydro-2H-pyran-2,3,4,5-tetrayl tetraacetate (1, 1.0 eq, 6.0 g, 12.4 mmol) and 4-nitrothiophenol (5.0 eq, 9.65 g, 62.2 mmol) in dichloromethane (80 mL), was added boron trifluoride diethyl etherate (10.0 eq, 15.2 mL, 124 mmol) at 0° C. The reaction mixture was stirred at room temperature for 16 h. After that, reaction mixture was quenched with ice water, extracted with dichloromethane. The organic layer washed with saturated bicarbonate solution, followed by water and dried over anhydrous sodium sulfate, filtered and concentrated to get crude. The crude was purified by flash column chromatography using 50-100% ethyl acetate in hexane as eluent to afford α:β isomer (7:3) (2R,3R,4S,5S,6R)-2-(2-(diethoxyphosphoryl)ethyl)-6-((4-nitrophenyl)thio)tetrahydro-2H-pyran-3,4,5-triyl triacetate (2) as a colorless sticky solid. Yield: 4.0 g, 55.7%; LC-MS, m/z. 578.14 [M+1]⁺.

### Synthesis of (2R,3S,4S,5R,6R)-2-((4-aminophenyl)thio)-6-(2-(diethoxyphosphoryl)ethyl)tetrahydro-2H-pyran-3,4,5-triyl triacetate (3)

To the stirred solution of (2R,3R,4S,5S,6R)-2-(2-(diethoxyphosphoryl)ethyl)-6-((4-nitrophenyl)thio)tetrahydro-2H-pyran-3,4,5-triyl triacetate (2, 1.0 eq, 1.2 g, 2.08 mmol) in dichloromethane (15.0 mL), 10% Palladium on carbon (0.62 g, 50% w/w) were added and reaction mixture was stirred under hydrogen (balloon pressure) at room temperature for 16 h. The progress of reaction was monitored by LC-MS and TLC. After the completion of reaction, reaction mixture was filtered through syringe filter. The filtrate was concentrated under reduced pressure bath temperature <35° C.) to afford crude mixture of α:β isomer (7:3) (2R,3S,4S,5R,6R)-2-((4-aminophenyl)thio)-6-(2-(diethoxyphosphoryl)ethyl)tetrahydro-2H-pyran-3,4,5-triyl triacetate (α isomer) and (2R,3S,4S,5R,6R)-2-((4-aminophenyl)thio)-6-(2-(diethoxyphosphoryl)ethyl)tetrahydro-2H-pyran-3,4,5-triyl triacetate (β isomer). The crude mixture was purified by prep-HPLC using (10-35% MeCN in water with 0.1% TFA). Fractions containing the desired product were combined and lyophilized to dryness to afford (2R,3S,4S,5R,6R)-2-((4-aminophenyl)thio)-6-(2-(diethoxyphosphoryl)ethyl)tetrahydro-2H-pyran-3,4,5-triyl triacetate (3) as off white solid. Yield: 0.65 g, 57%, α isomer; 0.2 g, 18%, β isomer LC-MS, m/z. 547.97 [M+1]⁺.

### Synthesis of (2R,3R,4S,5S,6R)-2-(2-(diethoxyphosphoryl)ethyl)-6-((4-(3-(hex-5-yn-1-yl)ureido)phenyl)thio)tetrahydro-2H-pyran-3,4,5-triyl triacetate (4)

To a solution of (2R,3S,4S,5R,6R)-2-((4-aminophenyl)thio)-6-(2-(diethoxyphosphoryl)ethyl)tetrahydro-2H-pyran-3,4,5-triyl triacetate (3, 1.0 eq, 0.65 g, 1.19 mmol) in N,N-dimethyl formamide (5.0 mL) were added N,N-diisopropylethyl amine (1.0 eq, 0.20 mL, 1.19 mmol) and 4-nitrophenyl hex-5-yn-1-ylcarbamate (3a, 1.20 eq, 0.37 g, 1.42 mmol) solution in N,N-dimethyl formamide (3.0 mL). The reaction mixture was stirred at room temperature for 16 h. The reaction mixture was then concentrated under reduced pressure to afford crude. The crude was purified by reverse phase (Aq C-18 column) column chromatography using 20-50% acetonitrile in water. The fractions were extracted with ethyl acetate and separated. The organic layer dried over anhydrous sodium sulfate, filtered and concentrated under reduced pressure to afford (2R,3R,4S,5S,6R)-2-(2-(diethoxyphosphoryl)ethyl)-6-((4-(3-(hex-5-yn-1-yl)ureido)phenyl)thio)tetrahydro-2H-pyran-3,4,5-triyl triacetate (4) as brown sticky solid; Yield: 0.33 g, 41.4%; LC-MS, m/z. 671.2 [M+1]⁺.

### Synthesis of (2-((2R,3R,4S,5S,6R)-3,4,5-triacetoxy-6-((4-(3-(hex-5-yn yl)ureido)phenyl)thio)tetrahydro-2H-pyran-2-yl)ethyl)phosphonic acid (5)

To a stirred solution of (2R,3R,4S,5S,6R)-2-(2-(diethoxyphosphoryl)ethyl)-6-((4-(3-(hex-5-yn-1-yl)ureido)phenyl)thio)tetrahydro-2H-pyran-3,4,5-triyl triacetate (4, 1.0 eq, 0.25 g, 0.373 mmol) in dichloromethane (8.0 mL), pyridine (10.0 eq, 0.30 mL, 3.73 mmol) and bromotrimethylsilane (10.0 eq, 0.49 mL, 3.73 mmol) was added at 0° C. and reaction mixture was stirred at room temperature for 16 h. After that, reaction mixture was quenched with ice water, extracted with dichloromethane. The organic layer dried over anhydrous sodium sulfate, filtered and concentrated under reduced pressure to get crude product. It was further washed with di-ethyl ether and dried to afford (2-((2R,3R,4S,5S,6R)-3,4,5-triacetoxy-6-((4-(3-(hex-5-yn-1-yl)ureido)phenyl)thio)tetrahydro-2H-pyran-2-yl)ethyl)phosphonic acid (5) as off white solid. Yield: 0.16 g, 69.84%; LC-MS, m/z. 614.93 [M+1]⁺.

### Synthesis of (2-((2R,3S,4S,5S,6R)-6-((4-(3-(hex-5-yn-1-yl)ureido)phenyl)thio)-3,4,5-trihydroxytetrahydro-2H-pyran-2-yl)ethyl)phosphonic acid (Cpd. No I-67)

To a stirred solution of (2-((2R,3R,4S,5S,6R)-3,4,5-triacetoxy-6-((4-(3-(hex-5-yn-1-yl)ureido)phenyl)thio)tetrahydro-2H-pyran-2-yl)ethyl)phosphonic acid (1.0 eq, 0.16 g, 0.260 mmol) in methanol (5.0 mL), sodium methoxide 25% w/v in methanol (7.0 eq, 0.40 mL, 1.82 mmol) was added drop-wise and reaction mixture was stirred at room temperature for 2 h. After that, reaction mixture was neutralized with Dowex hydrogen form (200-400 mesh) to pH-7. The reaction mixture was then filtered, concentrated under reduced pressure to give crude product. The crude material was purified by prep-HPLC using (eluting from a C18 column with 50-80% MeCN in water with 0.1% TFA). Fractions containing the desired product were combined and lyophilized to dryness to afford (2-((2R,3S,4S,5S,6R)-6-((4-(3-(hex-5-yn-1-yl)ureido)phenyl)thio)-3,4,5-trihydroxytetrahydro-2H-pyran-2-yl)ethyl)phosphonic acid (I-67) as white solid. Yield: 0.058 g, 45.61%; LC-MS, m/z 488.9 [M+1]⁺. ¹H NMR (400 MHz, DMSO-d₆) δ 8.51 (s, 1H), 7.37 (d, J=8.8 Hz, 2H), 7.30 (d, J=8.8 Hz, 2H), 6.18 (t, J=5.6 Hz, 1H), 5.16 (s, 1H) 5.10 (brs, 1H), 4.79 (brs, 1H), 3.86 (s, 1H), 3.70 (t, J=7.2 Hz, 1H), 3.42 (dd, J=9.2, 3.2 Hz, 1H), 3.39-3.29 (m, 2H), 3.09-3.06 (m, 2H), 2.76 (t, J=2.8 Hz, 1H), 2.20-2.17 (m, 2H), 2.03-2.01 (m, 1H), 1.63-1.31 (m, 7H).

### Example 68: Synthesis of (2-((2R,3S,4S,5S,6R)-6-((6-(hex-5-ynamido)naphthalen-2-yl)oxy)-3,4,5-trihydroxytetrahydro-2H-pyran-2-yl)ethyl)phosphonic acid (Cpd. No. I-68)

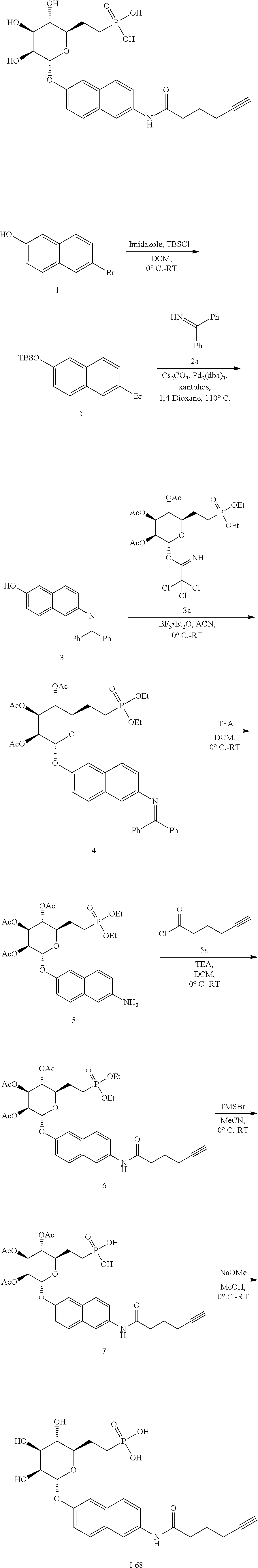

### Synthesis of ((6-bromonaphthalen-2-yl)oxy)(tert-butyl)dimethylsilane (2)

To a stirred solution of 6-bromonaphthalen-2-ol (1, 10.0 g, 1.0 eq., 44.8 mmol) in dichloromethane (50.0 mL), 1H-imidazole (6.1 g, 2.0 eq., 89.7 mmol) was added and the mixture was cooled to 0° C. tert-butyl(chloro)dimethylsilane (6.76 g, 1.0 eq., 44.8 mmol) was then added slowly. The reaction mixture was stirred at room temperature for 30 min and then diluted with dichloromethane and washed by water. Organic layer was separated, dried over anhydrous sodium sulfate, filtered and concentrated under reduced pressure to get crude which was purified by flash column chromatography using silica gel column (eluting with 5% ethyl acetate in hexane to afford ((6-bromonaphthalen-2-yl)oxy)(tert-butyl)dimethylsilane (2) as an off white solid. Yield: 12.0 g, 79.3%; ¹H NMR (400 MHz, DMSO-d₆) δ 8.09 (s, 1H), 7.79 (q, J=9.6 Hz, 2H), 7.53 (dd, J=8.8, 1.6 Hz, 1H), 7.31 (d, J=1.6 Hz, 1H), 7.14 (dd, J=8.8, 2.4 Hz, 1H), 0.92 (s, 9H), 0.22 (s, 6H).

### Synthesis of 6-((diphenylmethylene)amino)naphthalen-2-ol (3)

To stirred a solution of ((6-bromonaphthalen-2-yl)oxy)(tert-butyl)dimethylsilane (2, 4.0 g, 1.0 eq., 11.9 mmol) in 1,4-dioxane (40.0 mL), diphenylmethanimine (2.15 g, 1.0 eq., 11.9 mmol) and cesium carbonate (5.41 g, 1.40 eq., 16.6 mmol) was added at room temperature. Argon gas was purged in reaction mixture for 10 min and then xantphos (0.685 g, 0.1 eq., 1.19 mmol) and tris(1,5-diphenylpenta-1,4-dien-3-one) dipalladium (0.543 g, 0.05 eq., 0.593 mmol) were added. The reaction mixture was then transferred to a pre-heated (at 110° C.) heating bath and stirred the reaction for 12 h. Water was added and extracted with ethyl acetate. The organic layer was separated, dried over sodium sulfate, filtered and concentrated with reduced pressure to get crude material. The crude product was purified by flash colomn chromatography using silica gel column (30-40% ethyl acetate in hexane) to afford 6-[(diphenylmethylidene)amino]naphthalen-2-ol (3) as a yellow colored solid. Yield: (0.80 g, 20.8%); LCMS, m/z 322.1 [M−1]⁻.

### Synthesis of (2R,3R,4S,5S,6R)-2-(2-(diethoxyphosphoryl)ethyl)-6-((6-((diphenylmethylene)amino)naphthalen-2-yl)oxy)tetrahydro-2H-pyran-3,4,5-triyl triacetate (4)

To a cold (−78° C.) stirred solution of (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (3a, 1.50 g, 1.0 eq, 2.57 mmol) and 6-[(diphenylmethylidene)amino]naphthalen-2-ol (3, 0.830 g, 2.57 mmol) in dichloromethane (10.0 mL) was added boron trifluoride diethyl etherate (0.633 mL, 2 eq., 5.13 mmol) at −78° C., and then the reaction mixture was stirred for 4 h at 0° C. After that, reaction mixture was diluted with dichloromethane and washed with water. Organic layer was separated, dried over anhydrous sodium sulfate and concentrated to get crude which was purified by flash column chromatography (30-40% ethyl aceate in dichloromethane) to afford (2R,3R,4S,5S,6R)-2-(2-(diethoxyphosphoryl)ethyl)-6-((6-((diphenylmethylene)amino)naphthalen-2-yl)oxy)tetrahydro-2H-pyran-3,4,5-triyl triacetate (4), as yellow solid. Yield: 0.80 g, 42.0%; LC-MS, m/z 746.3 [M+1]⁺.

### Synthesis of (2R,3S,4S,5R,6R)-2-((6-aminonaphthalen-2-yl)oxy)-6-(2-(diethoxyphosphoryl)ethyl)tetrahydro-2H-pyran-3,4,5-triyl triacetate (5)

To a solution (2R,3R,4S,5S,6R)-2-(2-(diethoxyphosphoryl)ethyl)-6-((6-((diphenylmethylene)amino)naphthalen-2-yl)oxy)tetrahydro-2H-pyran-3,4,5-triyl triacetate (4, 0.80 g, 1.0 eq., 1.07 mmol) in dichloromethane (15.0 mL), trifluoroacetic acid (3.00 mL) was added at 0° C., and reaction mixture was stirred for 6 h at room temperature. After that, reaction mixture was concentrated under reduced pressure to get the crude compound. The crude compound was purified by trituration with diethyl ether and pentane solvents to give 2R,3S,4S,5R,6R)-2-((6-aminonaphthalen-2-yl)oxy)-6-(2-(diethoxyphosphoryl)ethyl)tetrahydro-2H-pyran-3,4,5-triyl triacetate (5) as a brown solid. Yield: 0.75 g, 60.0%, LC-MS, m/z-581.9, [M+1]⁺.

### Synthesis of (2R,3R,4S,5S,6R)-2-(2-(diethoxyphosphoryl)ethyl)-6-((6-(hex ynamido)naphthalen-2-yl)oxy)tetrahydro-2H-pyran-3,4,5-triyl triacetate (6)

To a solution of 2R,3S,4S,5R,6R)-2-((6-aminonaphthalen-2-yl)oxy)-6-(2-(diethoxyphosphoryl)ethyl)tetrahydro-2H-pyran-3,4,5-triyl triacetate (5, 0.80 g, 1.0 eq, 1.38 mmol) in dichloromethane (10.0 mL), triethylamine (0.580 mL, 3.0 eq., 4.13 mmol) and hex-5-ynoyl chloride (5a, 0.269 g, 1.50 eq., 2.06 mmol) were added at 0° C. and the reaction mixture was stirred for 4 h at room temperature. Water was added to the reaction mixture and extracted with dichloromethane. The combined organic fraction was dried over anhydrous sodium sulfate, filtered, and concentrated. The crude product was purified by flash column chromatography using silica gel column (using 3-4% methanol in dichloromethane) to afford (2R,3R,4S,5S,6R)-2-(2-(diethoxyphosphoryl)ethyl)-6-((6-(hex-5-ynamido)naphthalen-2-yl)oxy)tetrahydro-2H-pyran-3,4,5-triyl triacetate (6) as a brown solid. Yield: 0.70 g, 45.0%; LC-MS, m/z 676.0 [M+1]⁺.

### Synthesis of (2-((2R,3R,4S,5S,6R)-3,4,5-triacetoxy-6-((6-(hex-5-ynamido)naphthalen-2-yl)oxy)tetrahydro-2H-pyran-2-yl)ethyl)phosphonic acid (7)

To a solution of (2R,3R,4S,5S,6R)-2-(2-(diethoxyphosphoryl)ethyl)-6-((6-(hex-5-ynamido)naphthalen-2-yl)oxy)tetrahydro-2H-pyran-3,4,5-triyl triacetate (6, 0.70 g, 1.0 eq, 1.04 mmol) in dichloromethane (10.0 mL), pyridine (2.51 mL, 30 eq., 31.1 mmol) and bromotrimethylsilane (2.73 mL, 20 eq., 20.7 mmol) was added at 0° C. and the reaction mixture was stirred at room temperature for 3 h., After that, water was added and extracted with dichloromethane. The organic layer was dried over sodium sulfate, filtered and concentrated under reduced pressure to afford (2-((2R,3R,4S,5S,6R)-3,4,5-triacetoxy-6-((6-(hex-5-ynamido)naphthalen-2-yl)oxy)tetrahydro-2H-pyran-2-yl)ethyl)phosphonic acid (7) as pale yellow sticky gum. yield: 0.50 g, 77.9%; LC-MS, m/z 618.2 [M-1]⁻.

### Synthesis of (2-((2R,3S,4S,5S,6R)-6-((6-(hex-5-ynamido)naphthalen-2-yl)oxy)-3,4,5-trihydroxytetrahydro-2H-pyran-2-yl)ethyl)phosphonic acid (Cpd. No. I-68)

To a solution of (2-((2R,3R,4S,5S,6R)-3,4,5-triacetoxy-6-((6-(hex-5-ynamido)naphthalen-2-yl)oxy)tetrahydro-2H-pyran-2-yl)ethyl)phosphonic acid (7, 0.50 g, 0.807 mmol) in methanol (5.0 mL) was added 25% sodium methoxide solution (0.018 mL, 0.1 eq., 0.081 mmol) at 0° C. and the reaction mixture was stirred at room temperature for 1 h. After that, the reaction mixture was concentrated under reduced pressure to get crude compound which was purified by prep-HPLC (eluting from a C18 column with 30-40% acetonitrile in water with 0.1% TFA). The desired fractions were lyophilized to afford (2-((2R,3S,4S,5S,6R)-6-((6-(hex-5-ynamido)naphthalen-2-yl)oxy)-3,4,5-trihydroxytetrahydro-2H-pyran-2-yl)ethyl)phosphonic acid (Cpd. No. I-68) as a white solid. Yield: (0.188 g, 47.2%) LC-MS, m/z 494.1 [M+1]⁺. ¹H NMR (400 MHz, DMSO-d₆) δ 10.06 (s, 1H), 8.23 (s, 1H), 7.76-7.72 (m, 2H), 7.52 (dd, J=8.8, 2.0 Hz, 1H), 7.42 (d, J=2.4 Hz, 1H), 7.20 (dd, J=9.2, 2.4 Hz, 1H), 5.51 (d, J=1.6 Hz, 1H), 3.88-3.87 (m, 1H), 3.68 (dd, J=8.4, 3.2 Hz, 1H), 3.39-3.34 (m, 4H), 2.83 (t, J=2.4 Hz, 1H), 2.46 (t, J=7.2 Hz, 2H), 2.24 (td, J=6.8, 2.4 Hz, 2H), 1.96-1.93 (m, 1H), 1.82-1.75 (m, 2H), 1.63-1.48 (m, 2H), 1.17-1.05 (m, 1H).

### Example 69: 6-(3-aminopropyl)-2-(methylsulfonyl)nicotinonitrile hydrochloride (I-69)

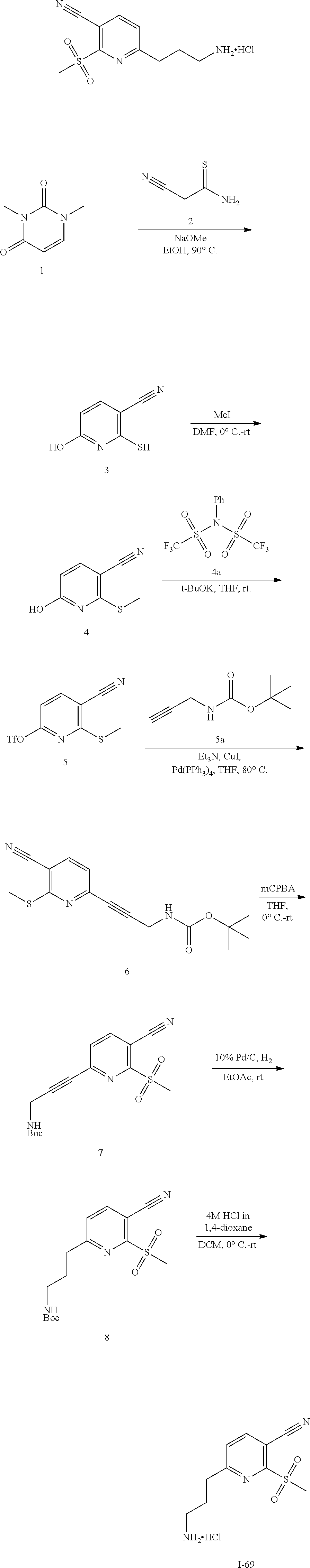

### Synthesis of 6-hydroxy-2-mercaptonicotinonitrile (3)

To a solution of 1,3-dimethylpyrimidine-2,4(1H,3H)-dione (1, 1.0 eq, 14.0 g, 99.9 mmol) in ethanol (150 mL), 25% sodium methoxide in methanol (2.0 eq, 44.0 mL, 200 mmol) and 2-cyanoethanethioamide (2, 1.0 eq, 10.0 g, 99.9 mmol) were added at room temperature, the resulting reaction mixture was stirred at 90° C. for 8 h. After completion, solvent was concentrated and residue was triturated with acetone, solid precipitated was filtered off and dried under vacuum to afford sodium 6-hydroxy-2-mercaptonicotinonitrile (3) as pale yellow solid. Yield:13.0 g, 74.75%; LCMS m/z 151.2 [M-1]−.

### Synthesis of 6-hydroxy-2-(methylthio)nicotinonitrile (4)

To a solution of 6-hydroxy-2-mercaptonicotinonitrile (3, 1.0 eq, 13.0 g, 74.6 mmol) in N,N-dimethylformamide (130 mL), iodomethane (4.65 mL, 1.0 eq., 74.6 mmol) was added at 0° C., the reaction mixture was stirred at room temperature for 30 min. After completion reaction, the reaction mixture was diluted with water and extract with ethyl acetate. The organic layer was dried over sodium sulfate, filtered, and concentrated under high vacuum to get crude. The crude was purified by flash column chromatography using 20-30% ethyl acetate in hexane to afford 6-hydroxy-2-(methylthio)nicotinonitrile (4) as pale yellow solid. Yield: 4.0 g, 32.24%; LCMS m/z 167.1 [M+1]⁺.

### Synthesis of 5-cyano-6-(methylthio)pyridin-2-yl trifluoromethanesulfonate (5)

To a solution of 1,1,1-trifluoro-N-phenyl-N-((trifluoromethyl)sulfonyl)methanesulfonamide (4a, 10.3 g, 1.2 eq., 28.9 mmol) in tetrahydrofuran (60.0 mL), potassium 2-methylpropan-2-olate (28.9 mL, 1.2 eq., 28.9 mmol) and 6-hydroxy-2-(methylthio)nicotinonitrile (4, 1.0 eq, 4.0 g, 24.1 mmol) were added at room temperature, the reaction mixture was stirred at room temperature for 16 h. After completion, the reaction mixture was diluted with water and extracted with ethyl acetate. The organic layer was dried over sodium sulfate, filtered, and concentrated under high vacuum to get crude. The crude was purified by flash Colum chromatography using 20-30% ethyl acetate in hexane to afford 5-cyano-6-(methylsulfanyl)pyridin-2-yl trifluoromethanesulfonate (5) as off white solid. Yield: 5.80 g, 80.8%; LCMS m/z 299.3 [M+1]+.

### Synthesis of tert-butyl (3-(5-cyano-6-(methylthio)pyridin-2-yl)prop-2-yn-1-yl)carbamate (6)

To a solution of 5-cyano-6-(methylthio)pyridin-2-yl trifluoromethanesulfonate (5, 1.0 eq, 5.80 g, 19.4 mmol) in tetrahydrofuran (40.0 mL), tert-butyl prop-2-yn-1-ylcarbamate (5a, 3.32 g, 1.1 eq., 21.4 mmol) and triethylamine (8.43 mL, 3 eq., 58.3 mmol) were at room temperature, the reaction mixture was degassed under nitrogen atmosphere. Palladium (2+) bis(triphenylphosphane) dichloride (0.682 g, 0.05 eq., 0.972 mmol) and copper(I) iodide (0.37 g, 0.1 eq., 1.94 mmol) were added. The reaction mixture was stirred at 80° C. for 3 h. After completion, the reaction mixture was diluted with water and extract with ethyl acetate, the organic layer was dried over sodium sulfate, filtered, and concentrated under high vacuum to get crude. The crude was purified by flash Colum chromatography using 20-30% ethyl acetate in hexane to afford tert-butyl (3-(5-cyano-6-(methylthio)pyridin-2-yl)prop-2-yn yl)carbamate (6) as pale yellow solid. Yield: 3.50 g, 59.32%; LCMS m/z 304.2 [M+1]+.

### Synthesis of tert-butyl (3-(5-cyano-6-(methylsulfonyl)pyridin-2-yl)prop-2-yn yl)carbamate (7)

To a solution of tert-butyl (3-(5-cyano-6-(methylthio)pyridin-2-yl)prop-2-yn yl)carbamate (6, 1.0 eq, 3.30 g, 10.9 mmol) in tetrahydrofuran (30 mL), 3-chlorobenzene carboperoxoic acid (8.64 g, 3 eq., 32.6 mmol) was added at 0° C., the reaction mixture was stirred at room temperature for 2 h. After completion, the reaction mixture was diluted with sodium bicarbonate solution and exacted with ethyl acetate. The organic layer was dried over sodium sulfate, filtered, and concentrated under high vacuum to get crude. The crude was purified by flash Colum chromatography using 30-50% ethyl acetate in hexane to afford tert-butyl (3-(5-cyano-6-(methylsulfonyl)pyridin-2-yl)prop-2-yn-1-yl)carbamate (7) as pale yellow oil. Yield: 2.0 g, 42.21%; LCMS m/z 336.4 [M+1]+.

### Synthesis of tert-butyl (3-(5-cyano-6-(methylsulfonyl)pyridin-2-yl)propyl)carbamate (8)

To a solution of tert-butyl (3-(5-cyano-6-(methylsulfonyl)pyridin-2-yl)prop-2-yn-1-yl)carbamate (7, 1.0 eq, 2.0 g, 5.96 mmol) in ethyl acetate (30.0 mL), 10% Palladium on carbon (1.0 g) was added at room temperature, the reaction mixture was stirred at room temperature under hydrogen atmosphere for 3 h. After completion, the reaction mixture was filtered through celite bed, filtrate was concentrated and dried under vacuum to afford tert-butyl (3-(5-cyano-6-(methylsulfonyl)pyridin-2-yl)propyl)carbamate (8) as pale yellow viscous liquid. Yield: 1.00 g, 42.98%; LCMS m/z 336.4 [M+1]⁺.

### Synthesis of 6-(3-aminopropyl)-2-(methylsulfonyl)nicotinonitrile hydrochloride (I-69)

To a solution of tert-butyl N-[3-(5-cyano-6-methanesulfonylpyridin-2-yl)propyl]carbamate (8, 1.00 g, 2.95 mmol) in dichloromethane (10.0 mL), 4M HCl in 1,4-dioxane (6.00 mL) was added at 0° C. The resulting reaction mixture was stirred at room temperature for 4 h. After completion, solvent was concentrated and dried to get crude, the crude was washed with diethyl ether and n-pentane and dried to afford 6-(3-aminopropyl)-2-methanesulfonylpyridine-3-carbonitrile hydrochloride (I-69) as off white solid. Yield: 0.785 g, 96.62%; LC-MS m/z 240.07 [M+1]⁺; ¹H-NMR (400 MHz, DMSO-d₆) δ 8.59 (d, J=8.0 Hz, 1H), 7.91 (bs, 3H), 7.85 (d, J=8.0 Hz, 1H), 3.56 (s, 1H), 3.47 (s, 3H), 3.04 (t, J=7.2 Hz, 1H), 2.87-2.82 (m, 2H), 2.06-1.99 (m, 2H).

### Example 70: (2-((2R,3S,4S,5S,6R)-3,4,5-trihydroxy-6-(4-(non-8-yn-1-yl)phenoxy)tetrahydro-2H-pyran-2-yl)ethyl)phosphonic acid (Cpd. No. I-70)

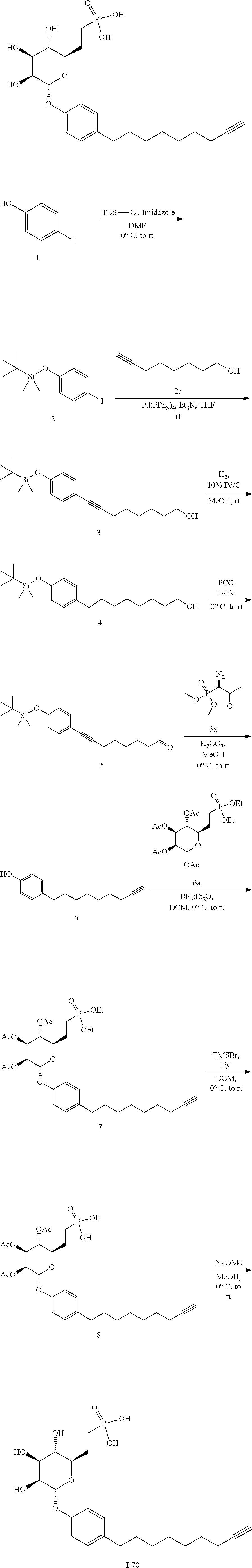

### Synthesis of tert-butyl(4-iodophenoxy)dimethylsilane (2)

To the stirred solution of 4-iodophenol (1, 10 g, 1.0 eq, 45.5 mmol) and imidazole (7.74 g, 2.50 eq, 114 mmol) in Dimethylformamide (75.00 mL) at 0° C., tert-Butyldimethylsilyl chloride (10.3 g, 1.5 eq, 68.2 mmol) was added portion-wise and reaction mixture stirred at room temperature for 16 h. After completion, reaction was diluted with water and extracted with ethyl acetate. Organic layer was dried using anhydrous sodium sulfate, filtered and concentrated under reduced pressure to get crude residue which was purified flash column chromatography on silica gel column using 5 to 10% Ethyl acetate in hexane as eluents. Desired fractions were concentrated under reduced pressure to afford tert-butyl(4-iodophenoxy) dimethylsilane (2) as colorless oil. Yield:14.0 g, 92.14%; ¹H NMR (400 MHz, CDCl₃) δ 7.49 (d, J=8.40 Hz, 2H), 6.60 (d, J=8.40 Hz, 2H), 0.96 (s, 9H), 0.18 (s, 6H).

### Synthesis of 8-(4-((tert-butyldimethylsilyl)oxy)phenyl)oct-7-yn-1-ol (3)

To a solution of tert-butyl(4-iodophenoxy)dimethylsilane (2, 7.95 g, 1.0 eq, 23.8 mmol) in tetrahydrofuran (120.0 mL) was added oct-7-yn-1-ol (2a, 3.00 g, 1.0 eq, 23.8 mmol), triethyl amine (10.0 mL, 3.0 eq, 71.3 mmol) and copper(I) iodide (0.45 g, 0.1 eq, 2.38 mmol) and reaction mixture purged with flow of argon gas for 15 minutes. tetrakis(triphenylphosphane) palladium (1.37 g, 0.05 eq, 1.19 mmol) was added to reaction mixture and reaction mixture stirred at room temperature for 16 h. Reaction mixture partitioned in between ethyl acetate and water. Ethyl acetate layer separated and washed with water, brine, dried over anhydrous sodium sulphate and concentrated under reduced pressure to get crude product. crude product obtained was purified by flash column chromatography on silica gel column eluting product in 10 to 30% ethyl acetate in hexane as eluents. Desired fractions were concentrated under reduced pressure to afford 8-(4-((tert-butyldimethylsilyl)oxy)phenyl)oct-7-yn-1-ol (3) brown color sticky gum. Yield: 5.20 g, 65.78%; LCMS m/z 333.30 [M+1]⁺

### Synthesis of 8-{4-[(tert-butyldimethylsilyl)oxy]phenyl}octan-1-ol (4)

To a solution of 8-(4-((tert-butyldimethylsilyl)oxy)phenyl)oct-7-yn-1-ol (3, 4.00 g, 1.0 eq, 12.0 mmol) in Methanol (30 mL) was added 10% palladium on carbon (0.400 g), Reaction mixture then stirred under hydrogen atmosphere at room temperature for 16 h. Completion of reaction was monitored by LCMS. The reaction mixture filtered over celite pad, filtrate obtained was concentrated under reduced pressure to afford 8-{4-[(tert-butyldimethylsilyl)oxy]phenyl}octan-1-ol (4) as colorless sticky gum. Yield: 3.90 g, 96%; ¹H NMR (400 MHz, CDCl₃) δ 7.00 (d, J=8.00 Hz, 2H), 6.73 (d, J=8.40 Hz, 2H), 3.65-3.58 (m, 2H), 2.51 (d, J=8.00 Hz, 2H), 1.55 (bs, 2H), 1.47 (bs, 2H), 1.31 (bs, 9H), 0.97 (s, 9H), 0.18 (s, 6H).

### Synthesis of 8-(4-((tert-butyldimethylsilyl)oxy)phenyl)octanal (5)

To a solution of 8-(4-((tert-butyldimethylsilyl)oxy)phenyl)octan-1-ol (4, 3.90 g, 1.0 eq, 11.6 mmol) in Dichloromethane (100 mL) at 0° C. was added Pyridinium chloro chromate (3.25 g, 1.3 eq, 15.1 mmol) and reaction mixture stirred at room temperature for 4 h. TLC showed formation of product. Reaction mixture filtered over celite pad and washed with ether. Filtrate concentrated under reduced pressure and crude obtained was column purified eluting compound in hexane to 5% ethyl acetate in hexane as eluents. Desired fractions were concentrated under reduced pressure to obtain 8-(4-((tert-butyldimethylsilyl)oxy)phenyl)octanal (5) as colorless oil. Yield: 2.60 g, 57.90%; LCMS m/z 335.35 [M+1]⁺

### Synthesis of 4-(non-8-yn-1-yl)phenol (6)

To a solution of 8-(4-((tert-butyldimethylsilyl)oxy)phenyl)octanal (5, 0.65 g, 1.0 eq, 1.94 mmol) in methanol (20.0 mL) at 0° C. was added potassium carbonate (0.805 g, 3 eq., 5.83 mmol) and 10% dimethyl (1-diazo-2-oxopropyl)phosphonate in Acetonitrile (5a, 7.46 mL, 2 eq, 3.89 mmol) and reaction mixture stirred at room temperature for 4 h. Reaction mixture quenched by addition of cold water and extracted with ethyl acetate. Ethyl acetate layer dried over anhydrous sodium sulphate and concentrated under reduced pressure to get crude compound. Crude compound obtained was purified by flash column chromatography using silica gel column eluting compound in 5 to 20% Ethyl acetate in hexane. The desired fractions were concentrated under reduced pressure to get 4-(non-8-yn-1-yl) phenol (6) as colorless sticky gum. Yield: 0.350 g, 83.28%; LCMS m/z 215.19[M−1]⁻

### Synthesis of (2R,3R,4S,5S,6R)-2-(2-(diethoxyphosphoryl)ethyl)-6-(4-(non-8-yn-1-yl)phenoxy)tetrahydro-2H-pyran-3,4,5-triyl triacetate (7)

To a stirred solution of 4-(non-8-yn-1-yl)phenol (6, 0.30 g, 1.0 eq, 1.39 mmol) and (3S,4S,5R,6R)-6-(2-(diethoxyphosphoryl)ethyl)tetrahydro-2H-pyran-2, 3,4,5-tetrayl tetraacetate (6a, 0.669 g, 1.0 eq, 1.39 mmol) in Dichloromethane (8.0 mL) was added activated molecular sieves (0.100 g) and reaction mixture stirred at room temperature for 15 mins. Reaction mixture cooled to 0° C. and borontrifluoride etherate (1.03 mL, 6 eq, 8.32 mmol) was added to reaction mixture and stirred at room temperature for 16 h. Reaction mixture cooled down and partitioned in between dichloromethane and aqueous sodium bicarbonate solution. Dichloromethane layer separated and washed with brine solution, dried over anhydrous sodium sulphate and concentrated under reduced pressure to get crude product. Crude product obtained was purified by combiflash column chromatography using silica gel column and eluting product in 30 to 50% Ethyl acetate in dichloromethane as eluents to afford (2R,3R,4S,5S,6R)-2-(2-(diethoxyphosphoryl)ethyl)-6-(4-(non-8-yn-1-yl)phenoxy)tetrahydro-2H-pyran-3,4,5-triyl triacetate (7) as colorless sticky gum. Yield: 0.35 g, 33.87%; LCMS m/z 639.49 [M+1]⁺

### Synthesis of (2-((2R,3R,4S,5S,6R)-3,4,5-triacetoxy-6-(4-(non-8-yn-1-yl)phenoxy)tetrahydro-2H-pyran-2-yl)ethyl)phosphonic acid (8)

To the stirred solution of (2R,3R,4S,5S,6R)-2-(2-(diethoxyphosphoryl)ethyl)-6-(4-(non-8-yn-1-yl)phenoxy)tetrahydro-2H-pyran-3,4,5-triyl triacetate (7, 0.35 g, 1.0 eq, 0.546 mmol) in dichloromethane (7.00 mL) at 0° C., Pyridine (0.663 mL, 15 eq., 8.22 mmol) and Bromotrimethylsilane (0.711 mL, 10 eq, 5.48 mmol) were added and reaction mixture was stirred at room temperature for 3 h and reaction was monitored by LCMS. After completion reaction mixture was diluted with water and concentrated under reduced pressure to get crude product. Crude product obtained was diluted with diethyl ether and filtered. Filtrate was concentrated under reduced pressure to afford (2-((2R,3R,4S,5S,6R)-3,4,5-triacetoxy (4-(non-8-yn-1-yl)phenoxy)tetrahydro-2H-pyran-2-yl)ethyl)phosphonic acid (8) as pale yellow sticky gum. yield: 0.25 g, 78%; LCMS m/z 581.35 [M−1]⁻

### Synthesis of(2-((2R,3S,4S,5S,6R)-3,4,5-trihydroxy-6-(4-(non-8-yn-1-yl)phenoxy)tetrahydro-2H-pyran-2-yl)ethyl)phosphonic acid (Cpd. No. I-70)

To a solution of (2-((2R,3R,4S,5S,6R)-3,4,5-triacetoxy-6-(4-(non-8-yn-1-yl)phenoxy)tetrahydro-2H-pyran-2-yl)ethyl)phosphonic acid (8, 0.25 g, 1.0 eq, 0.429 mmol) in Methanol (4.0 mL) at 0° C. was added Sodium methoxide solution (25%, 3 eq, 0.27 mL, 1.28 mmol) and reaction mixture stirred at room temperature for 3 h. LCMS showed formation of desired compound. Reaction mixture cooled down and neutralized with Dowex 50WX8 hydrogen form and filtered over sintered flask. Filtrate obtained was concentrated under reduced pressure to get crude product. Crude product obtained was purified by preparative HPLC (30-62% acetonitrile in water with 0.1% TFA) to afford (2-((2R,3S,4S,5S,6R)-3,4,5-trihydroxy-6-(4-(non-8-yn-1-yl)phenoxy)tetrahydro-2H-pyran-2-yl)ethyl)phosphonic acid (Cpd. No. I-70) as off white solid. Yield: 0.075 g, 38.29%, LCMS m/z 457.31 [M+1]⁺¹H NMR (400 MHz, DMSO-d₆) δ 7.08 (d, J=8.0 Hz, 2H), 6.92 (d, J=8.4 Hz, 2H), 5.00-4.74 (m, 3H), 3.79 (s, 1H), 3.63-3.60 (m, 1H), 3.39-3.28 (m, 6H), 2.72 (t, J=2.4 Hz, 2H), 2.14-2.10 (m, 2H), 1.91 (bs, 1H), 1.62-1.51 (m, 4H), 1.43-1.40 (m, 2H), 1.30-1.17 (m, 7H).

### Example 71: Synthesis of (2-((2R,3S,4S,5S,6R)-3,4,5-trihydroxy-6-(4-(oct-7-yn-1-yloxy)phenoxy)tetrahydro-2H-pyran-2-yl)ethyl)phosphonic acid (Cpd. No. I-71)

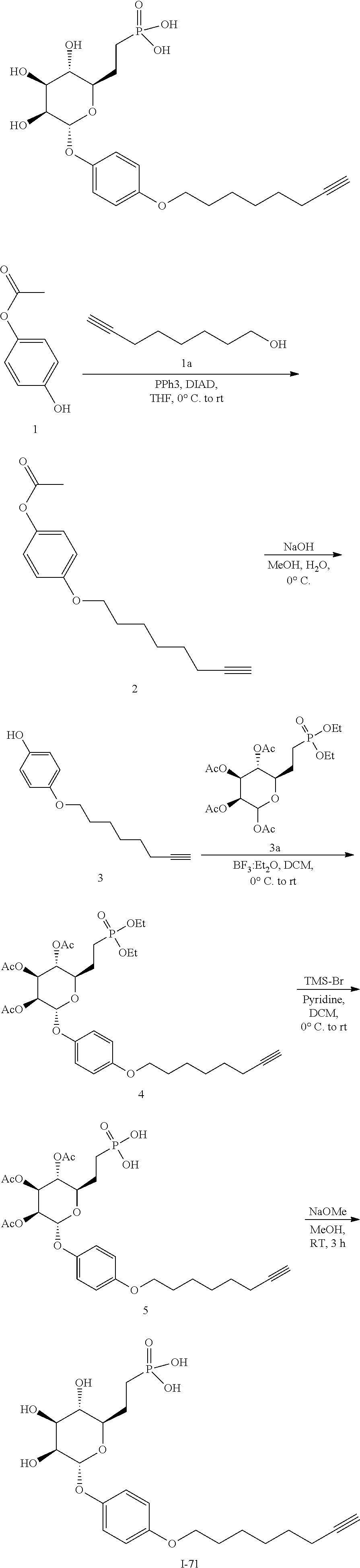

### Synthesis of 4-(oct-7-yn-1-yloxy)phenyl acetate (2)

To the stirred solution of 4-hydroxyphenyl acetate (1, 5.00 g, 1.0 eq, 0.032 mol) and oct-7-yn-1-ol (1a, 4.14 g, 1.0 eq, 0.032 mol) in tetrahydrofuran (50 mL) at 0° C., triphenyl phosphine (9.22 g, 1.1 eq, 0.035 mol) and diisopropyl azodicarboxylate (7.11 g, 1.1 eq, 0.035 mol) were added and reaction mixture stirred for 16 h at room temperature. After completion reaction mixture was diluted with water and extracted with ethyl acetate. Ethyl acetate layer was dried over anhydrous sodium sulfate and concentrated to get crude compound. The crude compound was purified by combi flash column chromatography using silica gel column and 5 to 7% ethyl acetate in hexane as eluents. Desired fractions were concentrated under reduced pressure to afford 4-(oct-7-yn-1-yloxy)phenyl acetate (2) as colorless liquid. Yield: 6.0 g, 70.13%; LC-MS m/z 259.18 [M−1]⁻.

### Synthesis of 4-(oct-7-yn-1-yloxy)phenol (3)

To the stirred solution of 4-(oct-7-yn-1-yloxy)phenyl acetate (2, 6.0 g, 1.0 eq, 0.023 mol) in methanol (36.0 mL) at 0° C., sodium hydroxide (1.84 g, 2.0 eq, 0.046 mol) dissolved in water (24.0 mL), was added and reaction mixture was stirred at same temperature for 30 min. After completion reaction mixture was concentrated under reduced pressure and then diluted with water and compound was extracted with ethyl acetate. Ethyl acetate layer was dried over anhydrous sodium sulfate and concentrated under reduced pressure to afford 4-(oct-7-yn-1-yloxy)phenol (3) as off white solid. Yield: 5.0 g, 99.38%; LC-MS m/z 217.14 [M−1]⁻.

### Synthesis of (2R,3R,4S,5S,6R)-2-(2-(diethoxyphosphoryl)ethyl)-6-(4-(oct-7-yn yloxy)phenoxy) tetrahydro-2H-pyran-3,4,5-triyl triacetate (4)

To a stirred solution of 4-(oct-7-yn-1-yloxy)phenol (3, 0.905 g, 3.0 eq, 4.15 mmol) and (2R,3S,4S,5R,6R)-6-(2-(diethoxyphosphoryl)ethyl)tetrahydro-2H-pyran-2,3,4,5-tetrayl tetraacetate (3a, 1.0 g, 1.0 eq, 1.39 mmol) in Dichloromethane (10.0 mL) was added activated molecular sieves (0.10 g) and reaction mixture stirred at room temperature for 15 mins. Reaction mixture cooled to 0° C. and borontrifluoride etherate (2.76 mL, 6 eq, 12.4 mmol) was added to reaction mixture and reaction mixture stirred at room temperature for 6 h. Reaction mixture cooled down and partitioned in between dichloromethane and aqueous sodium bicarbonate solution. Dichloromethane layer separated and washed with brine solution, dried over anhydrous sodium sulphate and concentrated under reduced pressure to get crude product. Crude product obtained was purified by combiflash column chromatography eluting product in 50-60% Ethyl acetate in hexane as eluents to afford (2R,3R,4S,5S,6R)-2-(2-(diethoxyphosphoryl)ethyl)-6-(4-(oct-7-yn-1-yloxy)phenoxy) tetrahydro-2H-pyran-3,4,5-triyl triacetate (4) as off white solid. Yield: 0.60 g, 45.18%; LC-MS m/z 641.26 [M+1]⁺.

### Synthesis of (2-((2R,3R,4S,5S,6R)-3,4,5-triacetoxy-6-(4-(oct-7-yn-1-yloxy)phenoxy)tetrahydro-2H-pyran-2-yl)ethyl)phosphonic acid (5)

To the stirred solution of (2R,3R,4S,5S,6R)-2-(2-(diethoxyphosphoryl)ethyl)-6-(4-(oct-7-yn-1-yloxy)phenoxy) tetrahydro-2H-pyran-3,4,5-triyl triacetate (4, 0.600 g, 1.0 eq, 0.937 mmol) in dichloromethane (10.0 mL) at 0° C. Pyridine (0.741 ml, 10.0 eq, 9.37 mmol) was added and stirred for 5 min. Bromotrimethylsilane (1.24 ml, 10.0 eq, 9.37 mmol) was added dropwise in reaction mixture. Reaction was stirred at room temperature for 3 h and reaction was monitored by LCMS. Reaction mixture was diluted with water and dichloromethane. Dichloromethane layer separated and aqueous layer re-extracted dichloromethane. Combined dichloromethane was dried over anhydrous sodium sulfate and concentrated under reduced pressure to afford (2-((2R,3R,4S,5S,6R)-3,4,5-triacetoxy-6-(4-(oct-7-yn-1-yloxy)phenoxy)tetrahydro-2H-pyran-2-yl)ethyl)phosphonic acid (5) as yellow liquid. Yield 0.500 g, 84.31%; LC-MS m/z 583.44 [M−1]⁻

### Synthesis of (2-((2R,3S,4S,5S,6R)-3,4,5-trihydroxy-6-(4-(oct-7-yn-1-yloxy)phenoxy)tetrahydro-2H-pyran-2-yl)ethyl)phosphonic acid (Cpd. No. I-71)

To the solution of (2-((2R,3R,4S,5S,6R)-3,4,5-triacetoxy-6-(4-(oct-7-yn-1-yloxy)phenoxy)tetrahydro-2H-pyran-2-yl)ethyl)phosphonic acid (5, 0.50 g, 1.0 eq, 0.856 mmol) in methanol (6.00 mL) at 0° C., Sodium methoxide solution (0.94 mL, 5.0 eq, 4.280 mmol) was added drop wise and reaction mixture stirred at room temperature for 3 h. After completion reaction was quenched with Dowex 50WX8 hydrogen form and filtered on sintered funnel. Filtrate obtained was concentrated under reduced pressure to get crude compound. The crude compound was purified by reverse phase preparative HPLC (37-57% acetonitrile in water with 0.1% TFA) to afford (2-((2R,3S,4S,5S,6R)-3,4,5-trihydroxy-6-(4-(oct-7-yn-1-yloxy)phenoxy)tetrahydro-2H-pyran-2-yl)ethyl)phosphonic acid (Cpd. No. I-71) as off white solid. Yield: 0.202 g, 51.51%; LCMS m/z 459.27 [M+1]⁺, ¹H-NMR (400 MHz, DMSO-d₆) δ 6.94 (d, J=9.2 Hz, 2H), 6.88 (d, J=9.2 Hz, 2H), 5.23 (d, J=1.2 Hz, 1H), 4.98 (bs, 1H), 4.72 (bs, 1H), 3.88 (t, J=6.4 Hz, 2H), 3.79 (s, 1H), 3.60 (d, J=4.8 Hz, 1H), 3.34-3.30 (m, 2H), 2.73 (t, J=2.4 Hz, 1H), 2.17-2.13 (m, 2H), 1.96-1.93 (m, 1H), 1.66 (t, J=6.4 Hz, 2H), 1.62-1.40 (m, 9H), 1.23-1.12 (m, 1H).

### Example 72: (2-((2R,3S,4S,5S,6R)-3,4,5-trihydroxy-6-(4-(3-(4-(1-(2-(2-(3-(2-(3-oxo-3-(perfluorophenoxy)propoxy)ethyl)phenoxy)ethoxy)ethyl)-1H-1,2,3-triazol-4-yl)butyl)thioureido)phenoxy)tetrahydro-2H-pyran-2-yl)ethyl)phosphonic acid (Cpd. No. I-72)

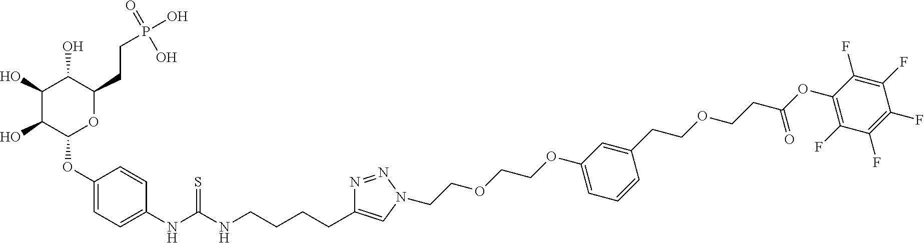 [SMILES: O=C(CCOCCc1cccc(OCCOCCn2cc(CCCCNC(=S)Nc3ccc(O[C@H]4O[C@H](CCP(=O)(O)O)[C@@H](O)[C@H](O)[C@@H]4O)cc3)nn2)c1)Oc1c(F)c(F)c(F)c(F)c1F]

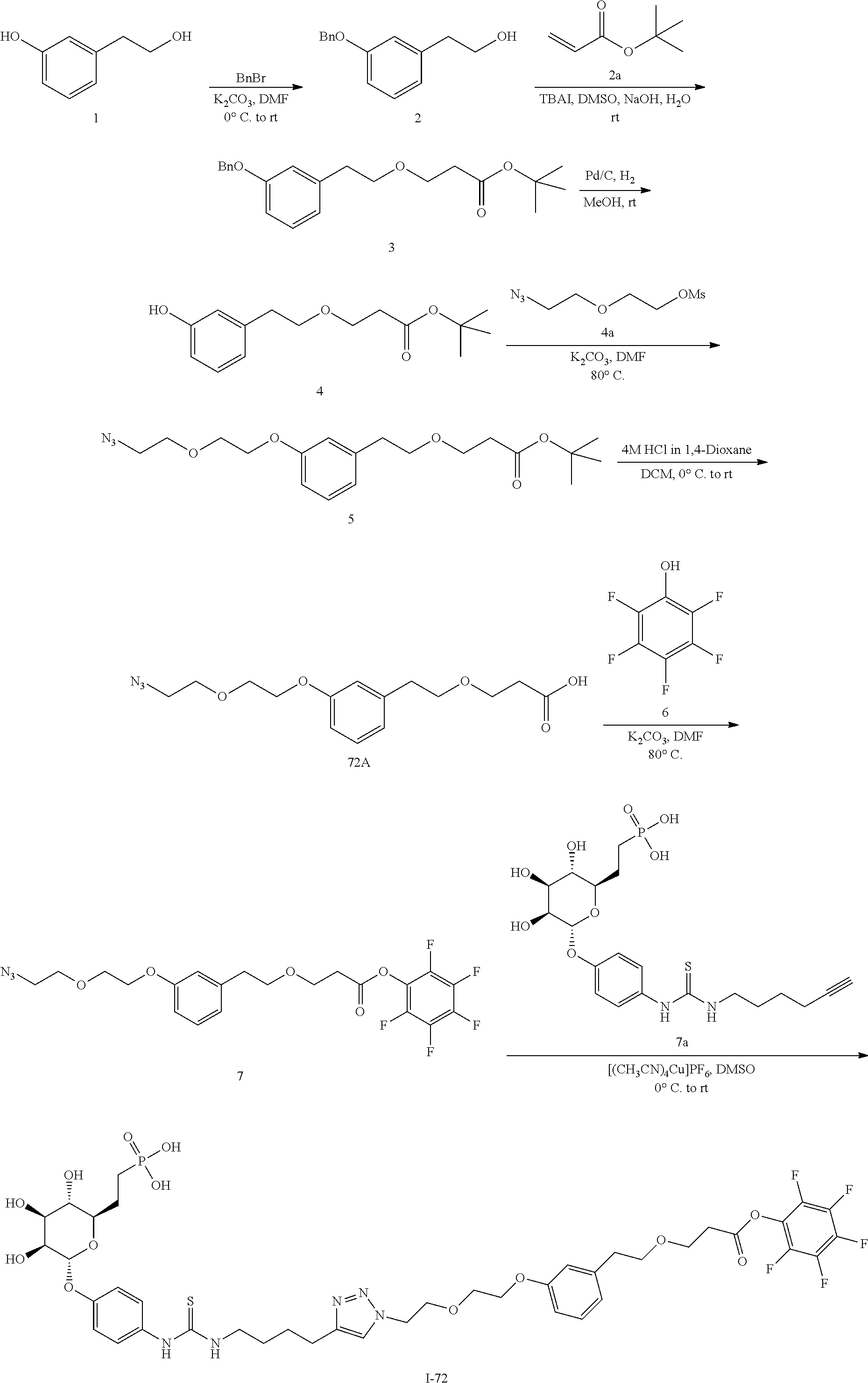

### Synthesis of 2-(3-(benzyloxy)phenyl)ethan-1-ol (2)

To a stirred solution of 3-(2-hydroxyethyl)phenol (1, 3.50 g, 1.0 eq, 25.3 mmol) in N,N-dimethylformamide (40 mL), potassium carbonate (7.00 g, 2 eq, 50.7 mmol) was added and reaction mixture cooled to 0° C. Benzyl bromide (6.02 mL, 2 eq, 50.7 mmol) was then added slowly and reaction mixture stirred at room temperature for 3h. After completion, reaction mixture was diluted with water and extracted with ethyl acetate. Organic layer was dried over anhydrous sodium sulfate, filtered and concentrated under reduced pressure to get crude product which was purified by flash column chromatography using silica gel column and 20% Ethyl acetate in hexane as eluents to afford of 2-(3-(benzyloxy)phenyl)ethan-1-ol (2) as colorless sticky gum. Yield: 5.0 g, 86%; LC-MS m/z 229.20 [M+1]⁺.

### Synthesis of tert-butyl 3-(3-(benzyloxy)phenethoxy)propanoate (3)

To a stirred solution of 2-[3-(benzyloxy)phenyl]ethan-1-ol (2, 5.00 g, 21.9 mmol) in Dimethylsufoxide (20.0 mL) at 0° C., sodium hydroxide (1.31 g, 1.5 eq, 32.9 mmol) dissolved in water (10.0 ml), tert-butyl prop-2-enoate (9.57 mL, 3 eq, 65.7 mmol), and tetrabutyl ammoniumiodide (1.62 g, 0.2 eq., 4.38 mmol) were added and reaction mixture stirred at room temperature for 4 h. After completion, reaction mixture was diluted with water and extracted with ethyl acetate. Ethyl acetate layer was dried over anhydrous sodium sulfate and concentrated under reduced pressure to get crude product which was purified by flash chromatography using silica gel column and 20% Ethyl acetate in hexanes as eluents. Desired fractions were concentrated under reduced pressure to afford tert-butyl 3-(3-(benzyloxy)phenethoxy)propanoate (3) as colorless sticky gum. Yield: 7.0 g, 89%; LC-MS m/z 355.29 [M−1]⁻.

### Synthesis of tert-butyl 3-(3-hydroxyphenethoxy)propanoate (4)

To a solution of tert-butyl 3-(3-(benzyloxy)phenethoxy)propanoate (3, 7.00 g, 19.6 mmol) in methanol (50 mL) was added 10% palladium on carbon (0.80 g) and reaction mixture stirred under hydrogen atmosphere for 3 h. After completion reaction mixture filtered over celite pad and filtrate was concentrated under reduced pressure to afford tert-butyl 3-(3-hydroxyphenethoxy)propanoate (4) as colorless sticky gum. Yield:4.2 g, 80%; LC-MS m/z 267.25 [M+1]⁺

### Synthesis of tert-butyl 3-(3-(2-(2-azidoethoxy)ethoxy)phenethoxy)propanoate (5)

To a solution of tert-butyl 3-(3-hydroxyphenethoxy)propanoate (4, 0.700 g, 2.63 mmol) in N,N-dimethylformamide (5.00 mL) was added potassium carbonate (1.09 g, 3 eq, 7.88 mmol) and 2-(2-azidoethoxy)ethyl methanesulfonate (4a, 0.660 g, 1.2 eq, 3.15 mmol) and reaction mixture was heated at 80° C. for 17 h. TLC showed consumption of starting material. Reaction mixture cooled down and quenched by addition of water and extracted with ethyl acetate. Ethyl acetate layer was washed with water, brine solution dried over anhydrous sodium sulphate and concentrated under reduced pressure to get crude product. Crude product obtained was purified by flash chromatography using silica gel column and eluting product in 15 to 20% ethyl acetate in hexane as eluents. Desired fractions were concentrated under reduced pressure to afford tert-butyl 3-(3-(2-(2-azidoethoxy)ethoxy)phenethoxy)propanoate (5) as colorless liquid. Yield: 0.50 g, 50%; LC-MS m/z 397.40 [M+18]⁺.

### Synthesis of 3-(3-(2-(2-azidoethoxy)ethoxy)phenethoxy)propanoic acid (72A)

To a solution of tert-butyl 3-(3-(2-(2-azidoethoxy)ethoxy)phenethoxy)propanoate (5, 0.400 g, 1.05 mmol) in dichloromethane (5.00 mL) at 0° C. was added 4N hydrochloric acid in 1,4-dioxane (5 mL) and reaction mixture was stirred at room temperature for 16 h, after completion reaction mixture was concentrated to get crude product which was purified by flash chromatography using silica gel column and 40% ethyl acetate in hexane as eluents. Desired fractions were concentrated under reduced pressure to afford 3-(3-(2-(2-azidoethoxy)ethoxy)phenethoxy)propanoic acid (Cpd. No. 72A) as colorless sticky gum. Yield: 0.183 g, 53%; LC-MS m/z 324.21 [M+18]⁺. ¹H-NMR (400 MHz, DMSO-d₆) δ 12.15 (s, 1H), 7.19-7.16 (m, 1H), 6.80-6.75 (m, 3H), 4.07 (t, J=4.4 Hz, 2H), 3.78-3.75 (m, 2H), 3.66 (t, J=4.8 Hz, 2H), 3.61-3.54 (m, 4H), 3.43-3.40 (m, 2H), 2.75 (t, J=7.2 Hz, 2H), 2.43 (t, J=6.40 Hz, 2H).

### Synthesis of perfluorophenyl 3-(3-(2-(2-azidoethoxy)ethoxy)phenethoxy)propanoate (7)

To a solution of 3-(3-(2-(2-azidoethoxy)ethoxy)phenethoxy)propanoic acid (Cpd. No. 72A, 0.200 g, 0.619 mmol) in ethyl acetate (2.0 mL) at 0° C. was added N,N′-Diisopropylcarbodiimide (0.097 mL, 0.619 mmol) and pentafluorophenol (6, 0.102 g, 0.9 eq, 0.557 mmol) and reaction mixture stirred at room temperature for 3 h. Reaction mixture filtered over celite bed and filtrate concentrated under reduced pressure to get crude product. Crude product obtained was purified by combiflash column chromatography using silica gel column and eluting compound in 0 to 10% Ethyl acetate in hexanes as eluents. Desired fractions were concentrated under reduced pressure to afford perfluorophenyl 3-(3-(2-(2-azidoethoxy)ethoxy)phenethoxy)propanoate (7) as colorless sticky gum. Yield: 0.13 g, 43%; ¹H-NMR (400 MHz, CDCl₃) δ 7.22-7.17 (m, 1H), 6.82-6.76 (m, 3H), 4.13-4.08 (m, 2H), 3.87-3.79 (m, 4H), 3.76-3.73 (m, 2H), 3.69-362 (m, 2H), 3.43-3.40 (m, 2H), 2.93-2.84 (m, 4H).

### Synthesis of (2-((2R,3S,4S,5S,6R)-3,4,5-trihydroxy-6-(4-(3-(4-(1-(2-(2-(3-(2-(3-oxo-3-(perfluorophenoxy)propoxy)ethyl)phenoxy)ethoxy)ethyl)-1H-1,2,3-triazol-4-yl)butyl)thioureido)phenoxy)tetrahydro-2H-pyran-2-yl)ethyl)phosphonic acid (Cpd. No. I-72)

To a solution of (2-((2R,3S,4S,5S,6R)-6-(4-(3-(hex-5-yn-1-yl)thioureido)phenoxy)-3,4,5-trihydroxytetrahydro-2H-pyran-2-yl)ethyl)phosphonic acid (7a, 0.043 g, 0.088 mmol) in dimethylsulfoxide (1.0 mL) was added perfluorophenyl 3-(3-(2-(2-azidoethoxy)ethoxy)phenethoxy)propanoate (7, 0.043 g, 1.0 eq, 0.088 mmol) in dimethylsulfoxide (0.5 mL) and reaction mixture cooled to 0° C. Tetrakis(acetonitrile)copper(I) hexafluorophosphate (0.082 g, 2.5 eq., 0.220 mmol) was added to reaction mixture and reaction mixture stirred at room temperature for 15 minutes. After completion reaction mixture was purified by reverse phase preparative HPLC using 30-70% acetonitrile in water with 0.1% TFA. Desired fractions were lyophilized to afford (2-((2R,3S,4S,5S,6R)-3,4,5-trihydroxy-6-(4-(3-(4-(1-(2-(2-(3-(2-(3-oxo-3-(perfluorophenoxy)propoxy)ethyl)phenoxy)ethoxy)ethyl)-1H-1,2,3-triazol-4-yl)butyl)thioureido)phenoxy)tetrahydro-2H-pyran-2-yl)ethyl)phosphonic acid (Cpd. No. I-72) as off white solid. Yield: 0.021 g, 24%; LC-MS m/z 978.36 [M+1]⁺. ¹H-NMR (400 MHz, DMSO-d6) δ 9.28 (s, 1H), 7.81 (s, 1H), 7.57 (bs, 1H), 7.25 (d, J=8.40 Hz, 2H), 7.15 (t, J=8.0 Hz, 1H), 6.98 (d, J=8.80 Hz, 2H), 6.80-6.78 (m, 2H), 6.73 (d, J=9.20 Hz, 2H), 5.32 (s, 1H), 4.48 (t, J=5.20 Hz, 2H), 4.01 (t, J=4.00 Hz, 2H), 3.84 (t, J=5.20 Hz, 2H), 3.80 (bs, 1H), 3.77-3.70 (m, 4H), 3.64-3.56 (m, 3H), 3.44 (bs, 2H), 3.36-3.28 (m, 2H), 3.01 (t, J=5.60 Hz, 2H), 2.77 (t, J=7.20 Hz, 2H), 2.59 (t, J=6.80 Hz, 2H), 1.96-1.92 (m, 1H), 1.55 (bs, 6H), 1.26-1.15 (m, 1H).

### Example 73: Synthesis of (2-((2R,3S,4S,5S,6R)-6-(4-(3-(hex-5-yn-1-yl)ureido)-2-methylphenoxy)-3,4,5-trihydroxytetrahydro-2H-pyran-2-yl)ethyl)phosphonic acid (Cpd. No. I-73)

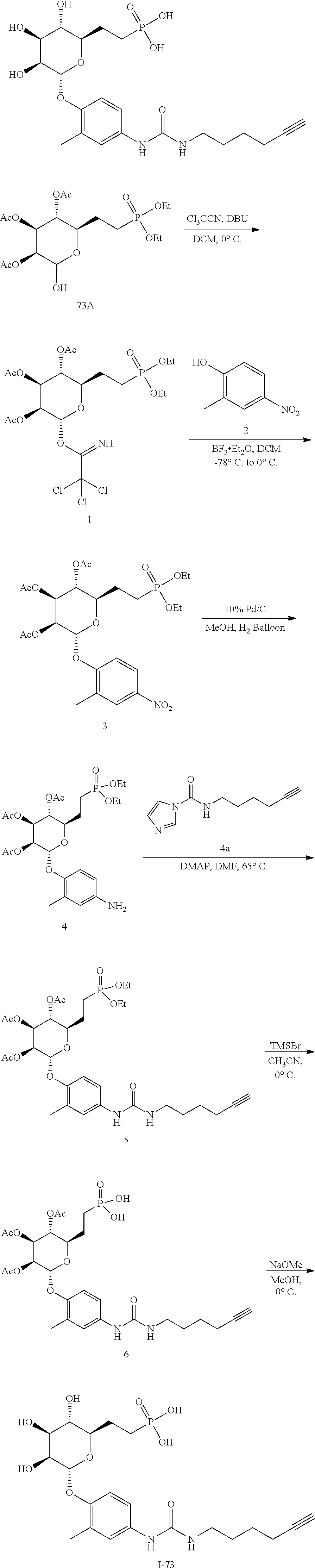

### Synthesis of (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (1)

1,8-Diazabicyclo[5.4.0]undec-7-ene (0.085 mL, 0.568 mmol) was added to a stirred solution of [(2R,3R,4S,5S,6S)-4,5-diacetoxy-2-(2-diethoxyphosphorylethyl)-6-hydroxy-tetrahydropyran-3-yl] acetate (73A, 2.5 g, 5.68 mmol) and trichloroacetonitrile (5.69 mL, 56.8 mmol) in dichloromethane (30.0 mL) at 0° C. under nitrogen. The resulting mixture was stirred at 0° C. under nitrogen. TLC at 30 min (100% ethyl acetate) shows conversion to less polar spot. Most of the solvent was removed on a rotary evaporator. The residue was loaded onto a silica gel loading column which was pre-equilibrated with 0.1% triethylamine in dichloromethane and purified via silica gel chromatography (column pre-equilibrated with 0.1% triethylamine in 20% ethyl acetate/dichloromethane) (20-100% ethyl acetate in dichloromethane) to afford (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (1) as a colorless semi-solid compound. Yield: 2.8 g, 84.35%.

### Synthesis of (2R,3R,4S,5S,6R)-2-(2-(diethoxyphosphoryl)ethyl)-6-(2-methyl nitrophenoxy)tetrahydro-2H-pyran-3,4,5-triyl triacetate (3)

[(2R,3R,4S,5S,6R)-4,5-diacetoxy-2-(2-diethoxyphosphorylethyl)-6-(2,2,2-trichloroethanimidoyl)oxy-tetrahydropyran-3-yl] acetate (1, 2.8 g, 4.79 mmol) was dissolved in dry dichloromethane (25 mL) with stirring under nitrogen. 2-methyl-4-nitrophenol (2, 1.83 g, 12.0 mmol) was added and the resulting clear solution was cooled to −78° C. with stirring under nitrogen. Boron trifluoride diethyl etherate (0.44 mL, 3.59 mmol) was added slowly. The −78° C. cold bath was removed and replaced with a 0° C. cold bath. Bright yellow color quickly faded. Reaction is a white cloudy mixture. The reaction mixture was stirred at 0° C. for 2 h. The reaction mixture was partitioned between dichloromethane and saturated aqueous sodium bicarbonate. The water layer was extracted again with dichloromethane. The combined organics were dried over sodium sulfate, filtered, concentrated on a rotary evaporator, and purified via silica gel chromatography (20-100% ethyl acetate in dichloromethane) to obtain (2R,3R,4S,5S,6R)-2-(2-(diethoxyphosphoryl)ethyl)-6-(2-methyl-4-nitrophenoxy)tetrahydro-2H-pyran-3,4,5-triyl triacetate (3) as viscous liquid. Yield: 1.5 g, 54.43%; LC-MS m/z 576.5 [M+1]⁺.

### Synthesis of (2R,3S,4S,5R,6R)-4,5-bis(acetyloxy)-2-(4-amino-2-methylphenoxy)-6-[2-(diethoxyphosphoryl)ethyl]oxan-3-yl acetate (4)

To a solution of (2R,3R,4S,5S,6R)-3,5-bis(acetyloxy)-2-[2-(diethoxyphosphoryl)ethyl]-6-(2-methyl-4-nitrophenoxy)oxan-4-yl acetate (3, 1.50 g, 2.61 mmol) in methanol (20.0 mL) was added 10% palladium carbon (0.6 g). The reaction mixture was stirred at room temperature for 1 h under hydrogen atmosphere. After completion, the reaction mixture was filtered through Syringe filter, filtrate was concentrated and dried to get (2R,3S,4S,5R,6R)-4,5-bis(acetyloxy)-2-(4-amino-2-methylphenoxy)-6-[2-(diethoxyphosphoryl)ethyl]oxan-3-yl acetate (4) as light pink liquid. Yield: 1.2 g, 84.4%; LC-MS m/z 546.46 [M+1]⁺.

### Synthesis of (2R,3S,4S,5R,6R)-4,5-bis(acetyloxy)-6-[2-(diethoxyphosphoryl)ethyl]-2-(4-{[(hex-5-yn-1-yl)carbamoyl]amino}-2-methylphenoxy)oxan-3-yl acetate (5)

To a solution of (2R,3S,4S,5R,6R)-4,5-bis(acetyloxy)-2-(4-amino-2-methylphenoxy)-6-[2-(diethoxyphosphoryl)ethyl]oxan-3-yl acetate (4, 1.20 g, 2.20 mmol) in N,N-dimethyl formamide (15.0 mL) was added N-(hex-5-yn-1-yl)-1H-imidazole-1-carboxamide (4a, 0.505 g, 2.64 mmol) and 4-dimethylaminopyridine (0.269 g, 2.20 mmol). The reaction mixture was stirred at 60° C. for 24 h. After completion, the reaction mixture was diluted with water and extracted with ethyl acetate. The organic layer was dried over sodium sulfate, filtered and concentrated under reduced pressure to get crude. The crude was purified by flash chromatography (silica mesh: 100-200; (elution: 3-5% methanol in dichloromethane) to obtain (2R,3S,4S,5R,6R)-4,5-bis(acetyloxy)-6-[2-(diethoxyphosphoryl)ethyl]-2-(4-{[(hex-5-yn-1-yl)carbamoyl]amino}-2-methylphenoxy)oxan yl acetate (5) as a pale yellow sticky liquid. Yield: 1.10 g, 74.78%; LC-MS m/z 669.2 [M+1]⁺.

### Synthesis of {2-[(2R,3R,4S,5S,6R)-3,4,5-tris(acetyloxy)-6-(4-{[(hex-5-yn-1-yl)carbamoyl]amino}-2-methylphenoxy)oxan-2-yl]ethyl}phosphonic acid (6)

To a solution of (2R,3S,4S,5R,6R)-4,5-bis(acetyloxy)-6-[2-(diethoxyphosphoryl)ethyl]-2-(4-{[(hex-5-yn-1-yl)carbamoyl]amino}-2-methylphenoxy)oxan yl acetate (5, 1.10 g, 1.65 mmol) in acetonitrile (15.0 mL) was added bromotrimethylsilane (1.09 mL, 8.23 mmol) at 0° C. The reaction mixture was stirred at room temperature for 5 h. After completion (monitored by LCMS), the reaction mixture was concentrated under reduced pressure to obtain sticky mass which was triturated with diethyl ether to obtain {2-[(2R,3R,4S,5S,6R)-3,4,5-tris(acetyloxy)-6-(4-{[(hex-5-yn-1-yl)carbamoyl]amino}-2-methylphenoxy)oxan-2-yl]ethyl}phosphonic acid (6) as crude compound which was used as such for next step without further purification. Yield: 1.0 g (crude); LCMS m/z 613.3 [M+1]⁺.

### Synthesis of {2-[(2R,3S,4S,5S,6R)-6-(4-{[(hex-5-yn-1-yl)carbamoyl]amino}-2-methylphenoxy)-3,4,5-trihydroxyoxan-2-yl]ethyl}phosphonic acid (Cpd. No. I-73)

To a solution of {2-[(2R,3R,4S,5S,6R)-3,4,5-tris(acetyloxy)-6-(4-{[(hex-5-yn-1-yl)carbamoyl]amino}-2-methylphenoxy)oxan-2-yl]ethyl}phosphonic acid (6, 1.00 g, 1.63 mmol) in methanol (10.0 mL) was added sodium methanolate (0.49 mL, 8.16 mmol) at 0° C. The reaction mixture was stirred at 0° C. to room temperature for 30 min. After completion (monitored by LCMS), the reaction mixture was concentrated under reduced pressure to obtain crude. The crude was purified by prep HPLC using (20-50% acetonitrile in water with 0.1% TFA) to afford {2-[(2R,3S,4S,5S,6R)-6-(4-{[(hex-5-yn-1-yl)carbamoyl]amino}-2-methylphenoxy)-3,4,5-trihydroxyoxan-2-yl]ethyl}phosphonic acid (Cpd. No. I-73) as off-white solid. Yield: 0.47 g, 59.94%; 487.5 [M+1]⁺; ¹H NMR (400 MHz, DMSO-d₆) δ 8.13 (s, 1H), 7.18 (d, J=2.0 Hz, 1H), 7.09 (dd, J=2.0, 8.4 Hz, 1H), 6.90 (d, J=8.8 Hz, 1H), 6.03 (t, J=5.2 Hz, 1H), 5.24 (s, 1H), 5.00 (bs, 2H), 4.72 (bs, 1H), 3.83 (s, 1H), 3.64 (d, J=6.0 Hz, 1H), 3.35-3.25 (m, 1H), 3.15 (s, 1H), 3.05 (t, J=6.0 Hz, 2H), 2.66 (s, 1H), 2.18 (t, J=4.0 Hz, 2H), 2.11 (s, 3H), 1.95 (bs, 1H), 1.65-1.58 (m, 1H), 1.47 (s, 6H), 1.23-1.13 (m, 1H).

### Example 74: (2-((2R,3S,4S,5S,6R)-6-(4-(3-(hex-5-yn-1-yl)ureido)-3-methylphenoxy)-3,4,5-trihydroxytetrahydro-2H-pyran-2-yl)ethyl)phosphonic acid (Cpd. No. I-74)

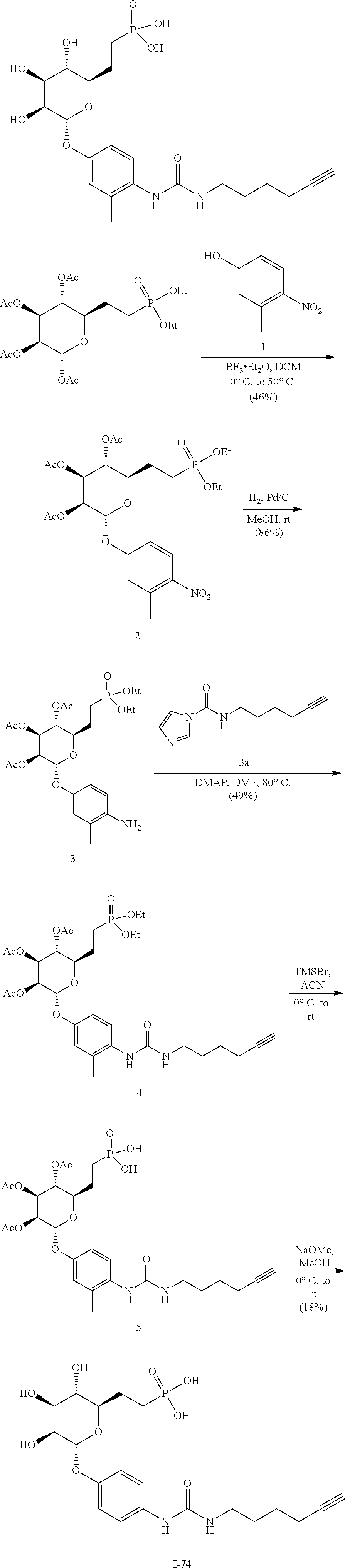

### Synthesis of (2R,3R,4S,5S,6R)-2-(2-(diethoxyphosphoryl)ethyl)-6-(3-methyl-4-nitrophenoxy)tetrahydro-2H-pyran-3,4,5-triyl triacetate (2)

A solution of (2R,3S,4S,5R,6R)-6-(2-(diethoxyphosphoryl)ethyl)tetrahydro-2H-pyran-2,3,4,5-tetrayl tetraacetate (1.0 eq, 2.0 g, 4.15 mmol) and 3-methyl-4-nitrophenol (1, 2.0 eq, 1.27 g, 8.29 mmol) in dichloromethane (20 mL) was cooled at 0° C., boron trifluoride diethyl etherate (5.0 eq, 2.67 mL, 20.7 mmol) was added dropwise and reaction mixture was heated at 50° C. for 16 h. After completion, reaction mixture was cooled at 0° C., quenched with saturated sodium bicarbonate solution and extracted with dichloromethane. Organic layer was dried over anhydrous sodium sulfate, filtered and concentrated to get crude which was purified by column chromatography using silica gel (100-200 mesh) and 0-40% ethyl acetate in dichloromethane to afford (2R,3R,4S,5S,6R)-2-(2-(diethoxyphosphoryl)ethyl)-6-(3-methyl-4-nitrophenoxy)tetrahydro-2H-pyran-3,4,5-triyl triacetate (2) as a brown viscous liquid. Yield: 1.1 g, 46.1%; LCMS m/z 576.35 [M+1]⁺.

### Synthesis of (2R,3S,4S,5R,6R)-2-(4-amino-3-methylphenoxy)-6-(2-(diethoxyphosphoryl)ethyl)tetrahydro-2H-pyran-3,4,5-triyl triacetate (3)

To a solution of (2R,3R,4S,5S,6R)-2-(2-(diethoxyphosphoryl)ethyl)-6-(3-methyl nitrophenoxy)tetrahydro-2H-pyran-3,4,5-triyl triacetate (2, 1.0 eq, 1.1 g, 1.91 mmol) in methanol (11 mL), Palladium on carbon (10%) (0.500 g) was added and reaction mixture was stirred under hydrogen gas atmosphere at room temperature for 2 h. After completion, reaction mixture was filtered, filtrate was concentrated and dried to afford (2R,3S,4S,5R,6R)-2-(4-amino-3-methylphenoxy)-6-(2-(diethoxyphosphoryl)ethyl)tetrahydro-2H-pyran-3,4,5-triyl triacetate (3) as a brown viscous liquid. Yield: 0.900 g, 86.41%; LCMS m/z 546.29 [M+1]⁺.

### Synthesis of (2R,3R,4S,5S,6R)-2-(2-(diethoxyphosphoryl)ethyl)-6-(4-(3-(hex-5-yn-1-yl)ureido)-3-methylphenoxy)tetrahydro-2H-pyran-3,4,5-triyl triacetate (4)

To a solution of (2R,3S,4S,5R,6R)-2-(4-amino-3-methylphenoxy)-6-(2-(diethoxyphosphoryl)ethyl)tetrahydro-2H-pyran-3,4,5-triyl triacetate (3, 1.0 eq, 0.600 g, 1.10 mmol) in N,N-dimethylformamide (6 mL), N-(hex-5-yn-1-yl)-1H-imidazole-1-carboxamide (3a, 1.2 eq, 0.252 g, 1.32 mmol) and 4-dimethylaminopyridine (1.0 eq, 0.134 g, 1.10 mmol) were added and reaction mixture was heated at 80° C. for 16 h. After completion, reaction mixture was cooled, water was added and extracted with ethyl acetate. Organic layer was washed with water, dried over anhydrous sodium sulphate, filtered and concentrated to get crude which was purified by column chromatography using silica gel (100-200 mesh) and 0-5% methanol in dichloromethane to afford (2R,3R,4S,5S,6R)-2-(2-(diethoxyphosphoryl)ethyl)-6-(4-(3-(hex-5-yn-1-yl)ureido)-3-methylphenoxy)tetrahydro-2H-pyran-3,4,5-triyl triacetate (4) as a colourless viscous liquid. Yield: 0.380 g, 49.29%; LCMS m/z 669.47 [M+1]⁺.

### Synthesis of (2-((2R,3R,4S,5S,6R)-3,4,5-triacetoxy-6-(4-(3-(hex-5-yn-1-yl)ureido)-3-methylphenoxy)tetrahydro-2H-pyran-2-yl)ethyl)phosphonic acid (5)

A solution of (2R,3R,4S,5S,6R)-2-(2-(diethoxyphosphoryl)ethyl)-6-(4-(3-(hex-5-yn-1-yl)ureido)-3-methylphenoxy)tetrahydro-2H-pyran-3,4,5-triyl triacetate (4, 1.0 eq, 0.600 g, 0.897 mmol) in dichloromethane (12 mL) was cooled at 0° C., bromotrimethylsilane (8.0 eq, 0.94 mL, 7.18 mmol) were added and reaction mixture was stirred at room temperature for 9 h. Reaction was monitored by LCMS. After completion, reaction mixture was concentrated and dried to afford (2-((2R,3R,4S,5S,6R)-3,4,5-triacetoxy-6-(4-(3-(hex-5-yn-1-yl)ureido)-3-methylphenoxy)tetrahydro-2H-pyran-2-yl)ethyl)phosphonic acid (5) as a brown viscous liquid. Yield: 0.590 g (Crude); LCMS m/z 613.27 [M+1]⁺.

### Synthesis of (2-((2R,3S,4S,5S,6R)-6-(4-(3-(hex-5-yn-1-yl)ureido)-3-methylphenoxy)-3,4,5-trihydroxytetrahydro-2H-pyran-2-yl)ethyl)phosphonic acid (Cpd. No. I-74)

A solution of (2-((2R,3R,4S,5S,6R)-3,4,5-triacetoxy-6-(4-(3-(hex-5-yn-1-yl)ureido)-3-methylphenoxy)tetrahydro-2H-pyran-2-yl)ethyl)phosphonic acid (5, 1.0 eq, 0.590 g, 0.963 mmol) in methanol (6 mL) was cooled at 0° C., sodium methoxide (25% solution in methanol) (10.0 eq, 2.36 mL, 9.63 mmol) was added and reaction mixture was stirred at room temperature for 1 h. After completion, reaction mixture was concentrated to get crude which was diluted with acetonitrile and purified by prep HPLC (23-41% acetonitrile in water with 0.1% TFA). Fractions containing the desired product were combined and lyophilized to dryness to afford (2-((2R,3S,4S,5S,6R)-6-(4-(3-(hex-5-yn-1-yl)ureido)-3-methylphenoxy)-3,4,5-trihydroxytetrahydro-2H-pyran-2-yl)ethyl)phosphonic acid (Cpd. No. I-74) as an off white solid. Yield: 0.085 g, 18.11%; LCMS m/z 487.13 [(M/2)+1]⁺; ¹H NMR (400 MHz, DMSO-d₆) δ 7.56-7.53 (m, 1H), 7.46 (s, 1H), 6.83 (s, 1H), 6.79-6.76 (m, 1H), 6.35-6.34 (m, 1H), 5.25 (s, 1H), 4.99-4.73 (m, 2H), 3.78 (s, 1H), 3.61-3.59 (m, 1H), 3.35-3.30 (m, 2H), 3.07-3.06 (m, 2H), 2.77-2.75 (m, 1H), 2.18 (bs, 2H), 2.13 (s, 3H), 1.96-1.95 (m, 1H), 1.60-1.57 (m, 1H), 1.48 (s, 5H), 1.23-1.14 (m, 1H).

### Example: 75 (2-((2R,3S,4S,5S,6R)-6-((6-(3-(hex-5-yn-1-yl)ureido)pyridin-3-yl)oxy)-3,4,5-trihydroxytetrahydro-2H-pyran-2-yl)ethyl)phosphonic acid (Cpd. No. I-75)

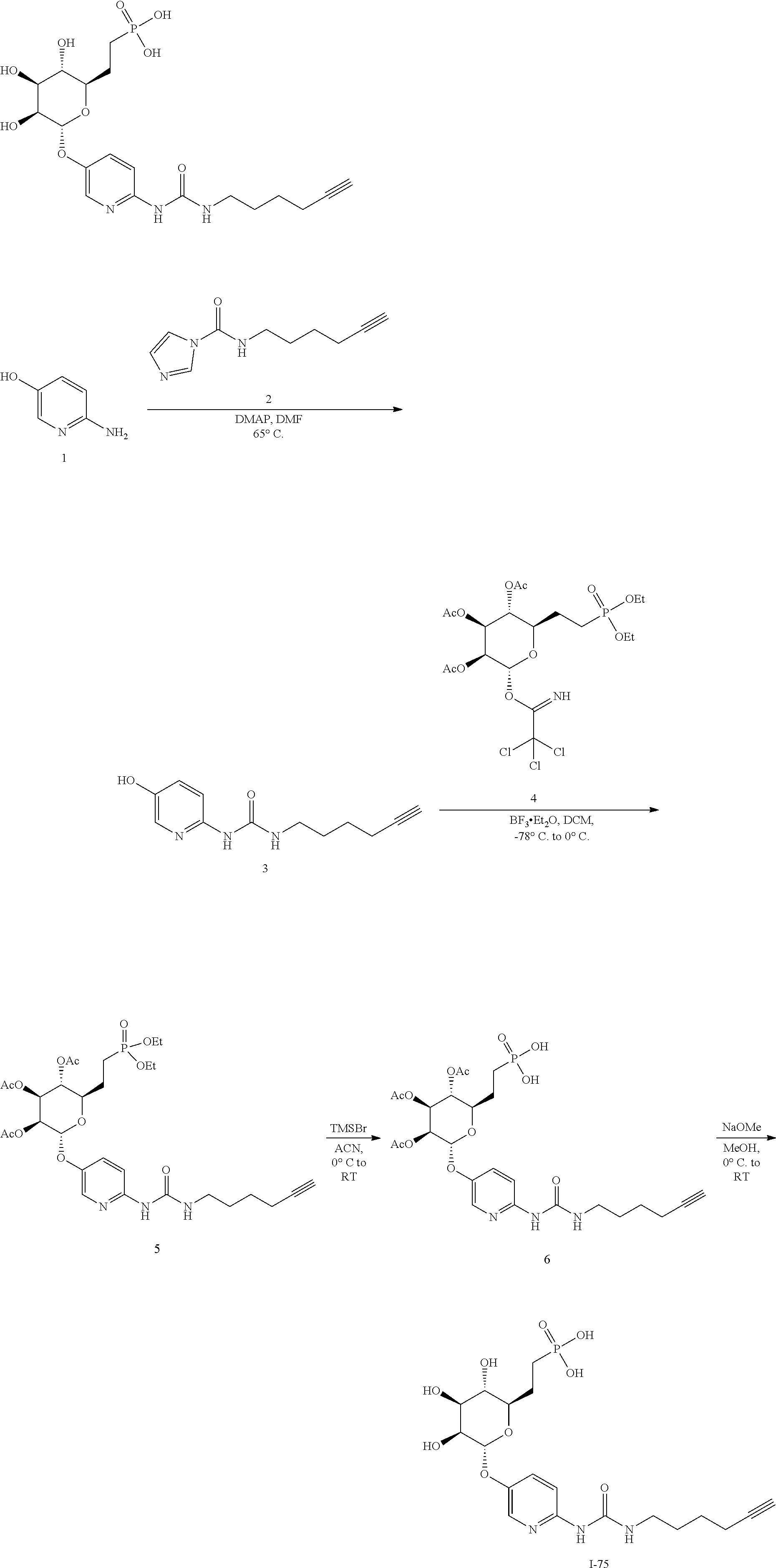

### Synthesis of 3-(hex-5-yn-1-yl)-1-(4-hydroxyphenyl)urea (3)

To a solution of 6-aminopyridin-3-ol (1, 1.5 g, 13.6 mmol) in N,N-dimethyl formamide (15.0 mL) was added N-(hex-5-yn-1-yl)-1H-imidazole-1-carboxamide (2, 2.6 g, 13.6 mmol) and N,N-dimethylpyridin-4-amine (1.66 g, 13.6 mmol). The reaction mixture was heated at 65° C. for 16 h. After completion, the reaction mixture was concentrated under reduced pressure to obtain crude. The crude was purified by column chromatography (silica mesh: 100-200; elution: 2-5% methanol in dichloromethane) to afford 3-(hex-5-yn-1-yl)-1-(4-hydroxyphenyl)urea (3) as yellow solid. Yield: 0.9 g, 28.32%; LC-MS m/z 234.12 [M+1]⁺.

### Synthesis of (2R,3R,4S,5S,6R)-2-(2-(diethoxyphosphoryl)ethyl)-6-((6-(3-(hex yn-1-yl)ureido)pyridin-3-yl)oxy)tetrahydro-2H-pyran-3,4,5-triyl triacetate (5)

In an inert atmosphere, (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (4, 1.0 g, 1.71 mmol) was dissolved in dry dichloromethane (10.0 mL) and stirred at room temperature. 3-(hex-5-yn-1-yl)-1-(4-hydroxyphenyl)urea (3, 0.4 g, 1.71 mmol) was added to the former solution and the resulting clear solution was cooled to −78° C. with stirring under nitrogen. Boron trifluoride diethyl etherate (0.21 mL, 1.71 mmol) was added drop-wise to the reaction vessel and the −78° C. cold bath was replaced with a 0° C. cold bath. The reaction mixture was stirred at 0° C. for 4 h and progress of reaction monitored with TLC and LC-MS. After completion, the reaction mixture was quenched with saturated aqueous sodium bicarbonate at 0° C. and partitioned between dichloromethane and aqueous layer. The aqueous layer was extracted again with dichloromethane (2×10 mL). The separated organic layers combined, dried over anhydrous sodium sulfate, filtered, concentrated on a rotary evaporator and purified by silica gel column chromatography (10% methanol in dichloromethane) to obtain (2R,3R,4S,5S,6R)-2-(2-(diethoxyphosphoryl)ethyl)-6-((6-(3-(hex-5-yn-1-yl)ureido)pyridin-3-yl)oxy)tetrahydro-2H-pyran-3,4,5-triyl triacetate (5) as viscous liquid. Yield: 0.12 g, 10.7%; LC-MS m/z 656.25 [M+1]⁺.

### Synthesis of (2-((2R,3R,4S,5S,6R)-3,4,5-triacetoxy-6-((6-(3-(hex-5-yn-1-yl)ureido)pyridin-3-yl)oxy)tetrahydro-2H-pyran-2-yl)ethyl)phosphonic acid (6)

To a solution of (2R,3R,4S,5S,6R)-2-(2-(diethoxyphosphoryl)ethyl)-6-((6-(3-(hex-5-yn-1-yl)ureido)pyridin-3-yl)oxy)tetrahydro-2H-pyran-3,4,5-triyl triacetate (5, 1.0 eq) in acetonitrile (10 vol.) is added bromotrimethylsilane (5.0 eq) at 0° C. The reaction mixture is stirred at room temperature for 5 h and progress monitored by TLC and LC-MS. After completion, the reaction mixture is concentrated under reduced pressure to obtain crude mass. The crude is washed with diethyl ether and decanted to obtain (2-((2R,3R,4S,5S,6R)-3,4,5-triacetoxy-6-((6-(3-(hex-5-yn-1-yl)ureido)pyridin-3-yl)oxy)tetrahydro-2H-pyran-2-yl)ethyl)phosphonic acid (6). LC-MS m/z 600.19 [M+1]⁺.

### Synthesis of (2-((2R,3S,4S,5S,6R)-6-((6-(3-(hex-5-yn-1-yl)ureido)pyridin-3-yl)oxy)-3,4,5-trihydroxytetrahydro-2H-pyran-2-yl)ethyl)phosphonic acid (Cpd. No. I-75)

To a solution of (2-((2R,3R,4S,5S,6R)-3,4,5-triacetoxy-6-((6-(3-(hex-5-yn yl)ureido)pyridin-3-yl)oxy)tetrahydro-2H-pyran-2-yl)ethyl)phosphonic acid (6, 1.0 eq) in methanol (10 vol.) is added sodium methoxide (10.0 eq) at 0° C. The reaction mixture is stirred at room temperature for 30 minutes and progress monitored by TLC. After completion, the reaction mixture is concentrated under reduced pressure to get crude. The crude is purified by prep-HPLC to afford dibenzyl (2-((2R,3S,4S,5S,6R)-6-((6-(3-(hex-5-yn yl)ureido)pyridin-3-yl)oxy)-3,4,5-trihydroxytetrahydro-2H-pyran-2-yl)ethyl)phosphonic acid (Cpd. No. I-75). LC-MS m/z 474.15 [M+1]⁺.

### Example 76: (2-((2R,3S,4S,5S,6R)-6-(4-azidophenoxy)-3,4,5-trihydroxytetrahydro-2H-pyran-2-yl)ethyl)phosphonic acid (Cpd. No. I-76)

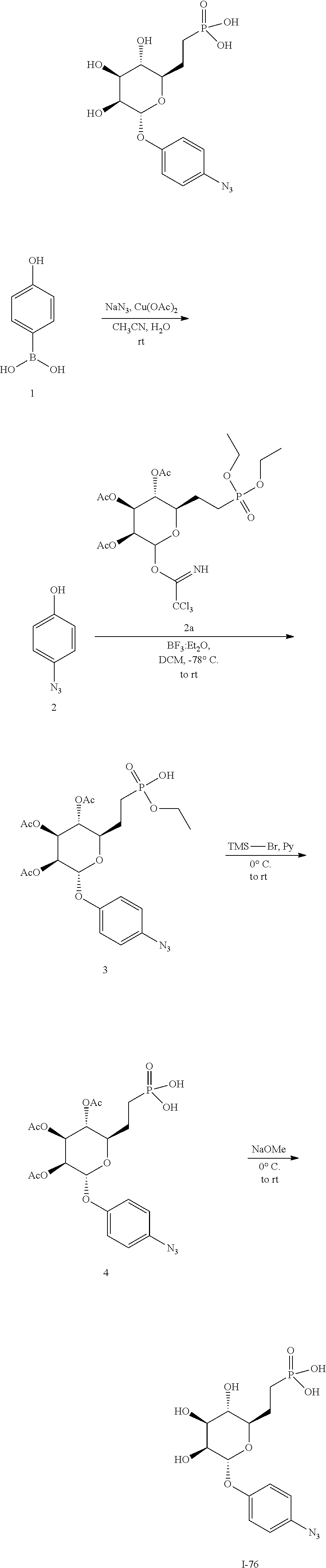

### Synthesis of 4-azidophenol (2)

To a solution of compound (4-hydroxyphenyl)boronic acid (1, 3.00 g, 1 eq, 21.8 mmol) and Sodium azide (2.12 g, 1.5 eq, 32.6 mmol) in mixture of acetonitrile (18.0 mL) and water (18.0 mL) was added copper(II) acetate (0.39 g, 0.1 eq, 32.6 mmol) and reaction mixture stirred at room temperature under air for 16 h. Reaction mixture partitioned in between ethyl acetate and water. Ethyl acetate layer separated and aqueous layer re-extracted with ethyl acetate. Combined ethyl acetate layer washed with brine solution, dried over anhydrous sodium sulphate, filtered and concentrated under reduced pressure to get crude product. Crude product obtained was purified by flash column chromatography on silica gel column eluting product in 20 to 30% ethyl acetate in hexane as eluents. Desired fractions were concentrated under reduced pressure to afford 4-azidophenol (2) as brownish sticky gum. Yield: 1.80 g, 61%; LCMS m/z 194.23 [M+60]⁻.

### Synthesis of (2R,3S,4S,5R,6R)-2-(4-azidophenoxy)-6-(2-(diethoxyphosphoryl)ethyl)tetrahydro-2H-pyran-3,4,5-triyl triacetate (3)

To a solution of 4-azidophenol (2, 0.324 g, 2.0 eq, 2.39 mmol) and [(2R,3R,4S,5S,6R)-4,5-diacetoxy-2-(2-diethoxyphosphorylethyl)-6-(2,2,2-trichloroethanimidoyl)oxy-tetrahydropyran-3-yl] acetate (2a, 0.700 g, 1.0 eq, 1.20 mmol) in dry dichloromethane (10 mL) at −78 C, Boron trifluoride diethyl etherate (0.111 mL, 0.75 eq, 0.898 mmol) was added slowly and reaction mixture was allowed to come at room temperature and stirred for 16 h. The reaction mixture was partitioned between dichloromethane and saturated aqueous sodium bicarbonate. The aqueous layer was re-extracted again with dichloromethane. The combined organics were dried over anhydrous sodium sulfate, filtered, and evaporated under reduced pressure to get crude residue. Crude product obtained was purified by flash column chromatography on silica gel column eluting product in 40 to 50% ethyl acetate in dichloromethane as eluents to afford (2R,3S,4S,5R,6R)-2-(4-azidophenoxy)-6-(2-(diethoxyphosphoryl)ethyl)tetrahydro-2H-pyran-3,4,5-triyl triacetate (3) as brownish sticky gum. Yield: 0.45 g, 67.43%; LCMS m/z 558.19 [M+1]⁺.

### Synthesis of (2-((2R,3R,4S,5S,6R)-3,4,5-triacetoxy-6-(4-azidophenoxy)tetrahydro-2H-pyran-2-yl)ethyl)phosphonic acid (4)

To a solution of (2R,3S,4S,5R,6R)-2-(4-azidophenoxy)-6-(2-(diethoxyphosphoryl)ethyl)tetrahydro-2H-pyran-3,4,5-triyl triacetate (3, 0.450 g, 1.0 eq, 0.807 mmol) in dichloromethane (10.0 mL) at 0° C. were added pyridine (0.977 mL, 15 eq, 12.1 mmol) and bromotrimethylsilane (1.07 mL, 10 eq, 8.07 mmol) and reaction mixture was stirred at room temperature for 4 h. LCMS showed consumption of starting material. Reaction mixture cooled to 0° C. and quenched by addition of cold water. Dichloromethane layer separated and Aqueous layer re-extracted with dichloromethane, combined dichloromethane layer dried over anhydrous sodium sulphate, filtered and concentrated under reduced pressure to afford (2-((2R,3R,4S,5S,6R)-3,4,5-triacetoxy-6-(4-azidophenoxy)tetrahydro-2H-pyran-2-yl)ethyl)phosphonic acid (4) as brownish sticky gum. Yield: 0.45 g, 80%; LCMS m/z 500.23 [M-1]⁻

### Synthesis of (2-((2R,3S,4S,5S,6R)-6-(4-azidophenoxy)-3,4,5-trihydroxytetrahydro-2H-pyran-2-yl)ethyl)phosphonic acid (Cpd. No. I-76)

To a solution of (2-((2R,3R,4S,5S,6R)-3,4,5-triacetoxy-6-(4-azidophenoxy)tetrahydro-2H-pyran-2-yl)ethyl)phosphonic acid (4, 0.405 g, 1.0 eq, 0.807 mmol) in methanol (5.0 mL) at 0° C. was added sodium methanolate (25% solution, 0.533 mL, 3 eq, 2.42 mmol) and reaction mixture stirred at room temperature for 1 h. LCMS showed consumption of Starting material. Reaction mixture neutralized with Dowex 50WX8 hydrogen form and filtered over sintered funnel. Filtrate obtained was concentrated under reduced pressure to get crude product. Crude product obtained was purified by reverse phase preparative H PLC using 13% to 35% acetonitrile in water with 0.1% trifluoroacetic acid to afford (2-((2R,3S,4S,5S,6R)-6-(4-azidophenoxy)-3,4,5-trihydroxytetrahydro-2H-pyran-2-yl)ethyl)phosphonic acid (Cpd. No. I-76) as cream color solid. Yield: 0.172 g, 56.78%; LCMS m/z 376.15 [M+1]⁺. ¹H NMR (400 MHz, DMSO-d6) δ 10.15 (bs, 1H), 7.10-7.04 (m, 5H), 5.05-4.77 (bm, 3H), 3.81 (s, 1H), 3.61 (d, J=8.0 Hz, 1H), 3.35-3.22 (m, 3H), 1.95-1.92 (bm, 1H), 1.61-1.45 (m, 2H), 1.17-1.05 (m, 1H).

### Example 77: (2-((2R,3S,4S,5S,6R)-6-(4-ethynylphenoxy)-3,4,5-trihydroxytetrahydro-2H-pyran-2-yl)ethyl)phosphonic acid (Cpd. No. I-77)

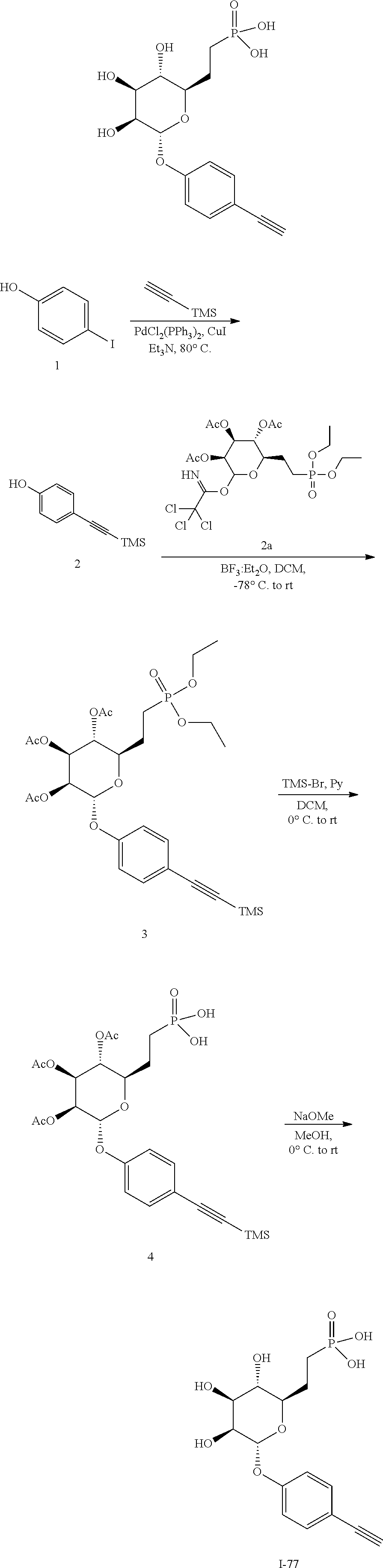

### Synthesis of 4-((trimethylsilyl)ethynyl)phenol (2)

To a solution of 4-lodophenol (1, 3.0 g, 1.0 eq, 13.6 mmol, 1 eq) in triethylamine (54.0 mL), copper (I) iodide (0.077 g, 0.409 mmol, 0.03 eq) was added and nitrogen gas was purged in reaction mixture for 10 minutes. Bis(triphenylphosphine)palladium(II) dichloride (0.287 g, 0.409 mmol, 0.03 eq), and trimethylsilylacetylene (3.0 mL, 20.5 mmol, 1.5 eq) were subsequently added into reaction mixture and reaction mixture heated at 80° C. for 3 h. Reaction mixture cooled down and concentrated under reduced pressure to get crude residue. Crude residue obtained was purified by flash column chromatography using silica gel column and 10 to 20% Ethyl acetate in hexane as eluents. Desired fractions were concentrated under reduced pressure to afford 4-[2-(trimethylsilyl)ethynyl]phenol (2) as brownish sticky gum. Yield: 2.58 g (99%); LCMS m/z 189.07 (M−1)⁻.

### Synthesis of (2R,3R,4S,5S,6R)-2-(2-(diethoxyphosphoryl)ethyl)-6-(4-((trimethylsilyl)ethynyl)phenoxy)tetrahydro-2H-pyran-3,4,5-triyl triacetate (3)

To a solution of (2R,3R,4S,5S,6R)-3,5-bis(acetyloxy)-2-[2-(diethoxyphosphoryl) ethyl]-6-[(2,2,2-trichloroethanimidoyl) oxy]oxan-4-yl acetate (2a, 1.40 g, 1.0 eq, 2.39 mmol) in dry dichloromethane (20.0 mL), 4-[2-(trimethylsilyl)ethynyl]phenol (2, 0.911 g, 2.0 eq, 4.79 mmol) was added and resulting solution was cooled to −78° C. Boron trifluoride diethyl etherate (0.222 mL, 0.75 eq, 1.80 mmol) was added slowly and reaction mixture was allowed to come at room temperature and stirred for 16 h. After completion of reaction, reaction mixture cooled down and partitioned in between dichloromethane and aqueous sodium bicarbonate solution. Dichloromethane layer separated and aqueous layer was re-extracted with dichloromethane. The combined organic layer was dried over anhydrous sodium sulfate, filtered, concentrated under reduce pressure, and purified by flash column chromatography using silica gel column and 20 to 30% ethyl acetate in dichloromethane as eluents. Desired fractions were concentrated under reduced pressure to obtain (2R,3R,4S,5S,6R)-2-(2-(diethoxyphosphoryl)ethyl)-6-(4-((trimethylsilyl)ethynyl)phenoxy)tetrahydro-2H-pyran-3,4,5-triyl triacetate (3) as pale yellow sticky gum. Yield: 0.710 g, 48.4%; LCMS m/z 613.28 [M+1]⁺.

### Synthesis of (2-((2R,3R,4S,5S,6R)-3,4,5-triacetoxy-6-(4-((trimethylsilyl)ethynyl)phenoxy)tetrahydro-2H-pyran-2-yl)ethyl)phosphonic acid (4)

To a solution of (2R,3R,4S,5S,6R)-2-(2-(diethoxyphosphoryl)ethyl)-6-(4-((trimethylsilyl)ethynyl)phenoxy)tetrahydro-2H-pyran-3,4,5-triyl triacetate (3, 0.610 g, 1.0 eq, 0.996 mmol) in dichloromethane (15.0 mL) at 0° C. were added pyridine (1.21 mL, 15 eq, 14.9 mmol) and bromotrimethylsilane (1.31 mL, 10 eq, 9.96 mmol) and reaction mixture stirred at room temperature for 4 h. LCMS showed consumption of starting material. Reaction mixture cooled to 0° C. and quenched by addition of cold water. Dichloromethane layer separated and aqueous layer re-extracted with dichloromethane. combined dichloromethane layer dried over anhydrous sodium sulphate and concentrated under reduced pressure to afford (2-((2R,3R,4S,5S,6R)-3,4,5-triacetoxy-6-(4-((trimethylsilyl)ethynyl)phenoxy)tetrahydro-2H-pyran-2-yl)ethyl)phosphonic acid (4) as brownish sticky gum. Yield:0.51 g, 92.3%; LCMS m/z 555.38 [M−1]⁻

### Synthesis of (2-((2R,3S,4S,5S,6R)-6-(4-ethynylphenoxy)-3,4,5-trihydroxytetrahydro-2H-pyran-2-yl)ethyl)phosphonic acid (Cpd. No. I-77)

To a solution of (2-((2R,3R,4S,5S,6R)-3,4,5-triacetoxy-6-(4-((trimethylsilyl)ethynyl)phenoxy)tetrahydro-2H-pyran-2-yl)ethyl)phosphonic acid (4, 0.510 g, 1.0 eq, 0.916 mmol) in methanol (8.00 mL) at 0° C. was added sodium methanolate (0.605 mL, 3 eq, 2.75 mmol) and reaction mixture stirred at room temperature for 4 h. Reaction mixture cooled and quenched by addition of Dowex 50W×8 hydrogen form up to pH 6 and filtered over sintered funnel. Filtrate obtained was concentrated under reduced pressure to get crude product. Crude product obtained was purified by reverse phase preparative HPLC using 10 to 35% acetonitrile in water and 0.1% TFA to afford (2-((2R,3S,4S,5S,6R)-6-(4-ethynylphenoxy)-3,4,5-trihydroxytetrahydro-2H-pyran-2-yl)ethyl)phosphonic acid (Cpd. No. I-77) as cream color solid. Yield: 0.213 g, 64%; LCMS m/z 359.06 [M+1]⁺. ¹H NMR (400 MHz, DMSO-d6) δ 10.20 (bs, 1H), 7.41 (d, J=8.80 Hz, 2H), 7.03 (d, J=8.80 Hz, 2H), 5.44 (s, 1H), 5.08-4.78 (bm, 3H), 4.05 (s, 1H), 3.81 (s, 1H), 3.62 (d, J=6.40 Hz, 1H), 3.35-3.19 (m, 3H), 1.92 (bs, 1H), 1.60-1.49 (m, 2H), 1.14-1.05 (m, 1H).

### Example 78: Synthesis of (2-((2R,3S,4S,5S,6R)-6-(4-(3-(hex-5-yn-1-yl)ureido)-3-hydroxyphenoxy)-3,4,5-trihydroxytetrahydro-2H-pyran-2-yl)ethyl)phosphonic acid (Cpd. No. I-78)

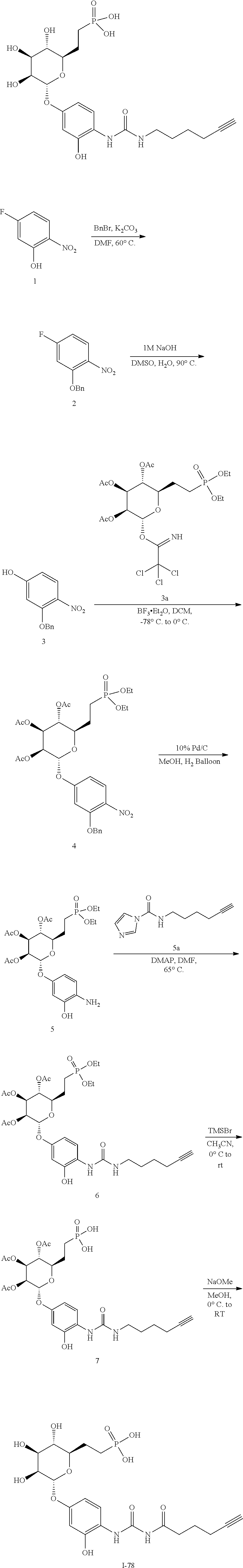

### Synthesis of 2-(benzyloxy)-4-fluoro-1-nitrobenzene (2)

To a solution of 5-fluoro-2-nitrophenol (1, 5.00 g, 1.0 eq, 31.8 mmol) in N, N-dimethylformamide (50.0 mL) were added potassium carbonate (5.28 g, 1.20 eq, 38.2 mmol) and benzyl bromide (4.16 mL, 35.0 mmol) and the reaction mixture was heated at 60° C. for 3 h. After completion, reaction mixture was diluted with water and extracted with ethyl acetate. The organic layer was dried over anhydrous sodium sulfate, filtered and concentrated under reduced pressure to afford 2-(benzyloxy)-4-fluoro-1-nitrobenzene (2) as yellow solid which was used as such for next step without further purification. Yield: 8.0 g, 99.64; LC-MS m/z 248.2 [M+1]⁺.

### Synthesis of 3-(benzyloxy)-4-nitrophenol (3)

To a solution of 2-(benzyloxy)-4-fluoro-1-nitrobenzene (2, 7.00 g, 28.3 mmol) in dimethylsulfoxide (35.00 mL) was added 1M sodium hydroxide solution in water (35.0 mL). The reaction mixture was stirred at 80° C. for 18 h. After completion (monitored by TLC), the reaction mixture was acidified with 1M hydrochloric acid (10 mL) until the pH 3-4 and the resultant solution was extracted with ethyl acetate. The organic layer was washed with water, dried over anhydrous sodium sulfate and concentrated to get crude. The crude was purified by flash column chromatography (silica mesh 100-200 mesh) using 15-20% ethyl acetate in hexane to afford 3-(benzyloxy)-4-nitrophenol (3) as yellow solid. Yield: 4.10 g, 59.05%; LC-MS m/z 246.2 [M+1]⁺.

### Synthesis of (2R,3S,4S,5R,6R)-4,5-bis(acetyloxy)-2-[3-(benzyloxy)-4-nitrophenoxy]-6-[2-(diethoxyphosphoryl)ethyl]oxan-3-yl acetate (4)

[(2R,3R,4S,5S,6R)-4,5-diacetoxy-2-(2-diethoxyphosphorylethyl)-6-(2,2,2-trichloroethanimidoyl)oxy-tetrahydropyran-3-yl] acetate (3a, 0.25 g, 1.0 eq 0.428 mmol) was dissolved in dry dichloromethane (2.5 mL) with stirring under nitrogen. 3-(benzyloxy)-4-nitrophenol (3, 0.105 g, 1.0 eq, 0.428 mmol) was added and the resulting clear solution was cooled to −78° C. with stirring under nitrogen. Boron trifluoride diethyl etherate (0.052 mL, 1.0 eq, 0.428 mmol) was added slowly. The −78° C. cold bath was removed and replaced with a 0° C. cold bath. The reaction mixture was stirred at 0° C. for 2 h. The reaction mixture was partitioned between dichloromethane and saturated aqueous sodium bicarbonate. The water layer was extracted again with dichloromethane. The combined organics were dried over anhydrous sodium sulfate, filtered, concentrated on a rotary evaporator, and purified via silica gel chromatography (5-10% methanol in dichloromethane) to obtain (2R,3S,4S,5R,6R)-4,5-bis(acetyloxy)-2-[3-(benzyloxy)-4-nitrophenoxy]-6-[2-(diethoxyphosphoryl)ethyl]oxan-3-yl acetate (4) as viscous liquid. Yield: 0.12 g (˜65% purity by LCMS); LC-MS m/z 668.6 [M+1]⁺.

### Synthesis of (2R,3S,4S,5R,6R)-2-(4-amino-3-hydroxyphenoxy)-6-(2-(diethoxyphosphoryl)ethyl)tetrahydro-2H-pyran-3,4,5-triyl triacetate (5)

To a solution of (2R,3S,4S,5R,6R)-4,5-bis(acetyloxy)-2-[3-(benzyloxy)-4-nitrophenoxy]-6-[2-(diethoxyphosphoryl)ethyl]oxan-3-yl acetate (4, 1.0 eq) in methanol (10 vol.) is added 10% palladium on carbon (quant.). The reaction mixture is stirred at room temperature for 3 h under hydrogen atmosphere. After completion, the reaction mixture was filtered through Syringe filter, filtrate is concentrated and dried to get (2R,3S,4S,5R,6R)-2-(4-amino-3-hydroxyphenoxy)-6-(2-(diethoxyphosphoryl)ethyl)tetrahydro-2H-pyran-3,4,5-triyl triacetate (5). LC-MS m/z 548.15 [M+1]⁺.

### Synthesis of (2R,3R,4S,5S,6R)-2-(2-(diethoxyphosphoryl)ethyl)-6-(4-(3-(hex yn-1-yl)ureido)-3-hydroxyphenoxy)tetrahydro-2H-pyran-3,4,5-triyl triacetate (6)

To a solution of (2R,3S,4S,5R,6R)-4,5-bis(acetyloxy)-2-(4-amino methylphenoxy)-6-[2-(diethoxyphosphoryl)ethyl]oxan-3-yl acetate (5, 1.0 eq) in N,N-dimethyl formamide (10 vol) is added N-(hex-5-yn-1-yl)-1H-imidazole-1-carboxamide (5a, 1.2 eq) and 4-dimethylaminopyridine (1.0 eq). The reaction mixture is stirred at 60° C. for 24 h. After completion, the reaction mixture is diluted with water and extracted with ethyl acetate. The organic layer is dried over anhydrous sodium sulfate, filtered and concentrated under reduced pressure to get crude. The crude is purified by flash chromatography (silica mesh: 100-200) and 5 to 10% methanol in dichlomethane as eluents to afford (2R,3R,4S,5S,6R)-2-(2-(diethoxyphosphoryl)ethyl)-6-(4-(3-(hex-5-yn-1-yl)ureido)-3-hydroxyphenoxy)tetrahydro-2H-pyran-3,4,5-triyl triacetate (6). LC-MS m/z 671.25 [M+1]⁺.

### Synthesis of (2-((2R,3R,4S,5S,6R)-3,4,5-triacetoxy-6-(4-(3-(hex-5-yn-1-yl)ureido)-3-hydroxyphenoxy)tetrahydro-2H-pyran-2-yl)ethyl)phosphonic acid (7)

To a solution of (2R,3R,4S,5S,6R)-2-(2-(diethoxyphosphoryl)ethyl)-6-(4-(3-(hex-5-yn-1-yl)ureido)-3-hydroxyphenoxy)tetrahydro-2H-pyran-3,4,5-triyl triacetate (6, 1.0 eq) in acetonitrile (10 vol.) is added bromotrimethylsilane (5.0 eq) at 0° C. The reaction mixture is stirred at room temperature for 5 h. After completion, the reaction mixture is concentrated under reduced pressure to obtain sticky mass which is triturated with diethyl ether to obtain (2-((2R,3R,4S,5S,6R)-3,4,5-triacetoxy-6-(4-(3-(hex-5-yn-1-yl)ureido)-3-hydroxyphenoxy)tetrahydro-2H-pyran-2-yl)ethyl)phosphonic acid (7) as crude compound which is used as such for next step without further purification. LC-MS m/z 615.15 [M+1]⁺.

### Synthesis of (2-((2R,3S,4S,5S,6R)-6-(4-(3-(hex-5-yn-1-yl)ureido)-3-hydroxyphenoxy)-3,4,5-trihydroxytetrahydro-2H-pyran-2-yl)ethyl)phosphonic acid (Cpd. No. I-78)

(2-((2R,3R,4S,5S,6R)-3,4,5-triacetoxy-6-(4-(3-(hex-5-yn-1-yl)ureido)-3-hydroxyphenoxy)tetrahydro-2H-pyran-2-yl)ethyl)phosphonic acid (7, 1.0 eq) in methanol (10 vol.) is added sodium methanolate (10.0 eq) at 0° C. The reaction mixture is stirred at 0° C. to room temperature for 30 min. After completion, the reaction mixture is neutralized by Dowex 50WX8 hydrogen form up to pH 6 to 7 and filtered. Filtrate is concentrated under reduced pressure to obtain crude. The crude is purified by reverse phase preparative HPLC to afford (2-((2R,3S,4S,5S,6R)-6-(4-(3-(hex-5-yn-1-yl)ureido)-3-hydroxyphenoxy)-3,4,5-trihydroxytetrahydro-2H-pyran-2-yl)ethyl)phosphonic acid (Cpd. No. I-78). LC-MS m/z 489.16 [M+1]⁺.

### Example 79: (2-((2R,3S,4S,5S,6R)-6-((2-(hex-5-ynamido)quinolin-6-yl)oxy)-3,4,5-trihydroxytetrahydro-2H-pyran-2-yl)ethyl)phosphonic acid (Cpd. No. I-79)

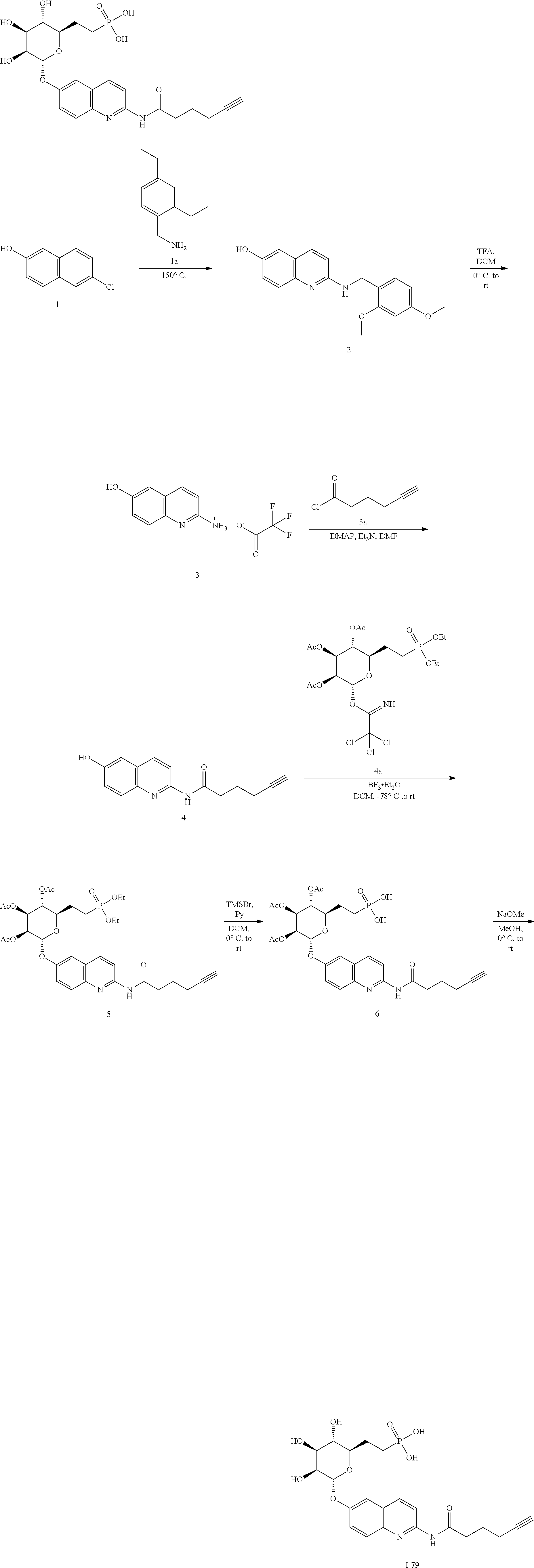

### Synthesis of 2-((2,4-dimethoxybenzyl)amino)quinolin-6-ol (2)

A solution of 2-chloroquinolin-6-ol (1, 1.0 g, 1.0 eq, 5.57 mmol) and (2,4-dimethoxyphenyl)methanamine (1a, 1.67 mL, 2.0 eq, 11.1 mmol) was heated at 150° C. for 16 h and progress of reaction was checked by TLC and LC-MS. After completion, reaction was concentrated and observed crude residue was purified by combiflash chromatography using silica gel column and 30 to 40% ethyl acetate in hexane as eluents to afford 2-((2,4-dimethoxybenzyl)amino)quinolin-6-ol (2) as pale yellow solid. Yield: 0.72 g (40.1%); LCMS m/z 311.18 (M+1)⁺.

### Synthesis of 2-aminoquinolin-6-ol trifluro acetic acid salt (3)

To a solution of 2-((2,4-dimethoxybenzyl)amino)quinolin-6-ol (2, 0.10 g, 0.32 mmol) in dichloromethane (0.5 mL) at 0° C. was added trifluoroacetic acid (0.5 mL) and reaction mixture stirred at room temperature for 6 h. Reaction mixture concentrated under reduced pressure to afford 2-aminoquinolin-6-ol trifluro acetic acid salt (3) as pale yellow solid. Yield: 0.080 g, 91.0%; LCMS m/z 160.86 [M+1]⁺.

### Synthesis of N-(6-hydroxyquinolin-2-yl)hex-5-ynamide (4)

To a solution of 2-aminoquinolin-6-ol trifluro acetic acid salt (3, 1.0 eq.) in N,N-dimethylformamide is added triethyl amine (0.12 mL, 3.0 eq, 0.87 mmol) and N,N-dimethylpyridin-4-amine (0.2 eq.). Reaction mixture is cooled to 0° C. and hex-5-ynoyl chloride (3a, 0.045 g, 1.2 eq, 0.34 mmol) is added to reaction mixture and stirred for 16 h and monitored by TLC and LC-MS for the completion. Reaction mixture partitioned in between ethyl acetate and water. Ethyl acetate layer separated and aqueous layer re-extracted with ethyl acetate. Ethyl acetate layer is dried over anhydrous sodium sulfate and concentrated to get crude residue. Crude residue obtained is purified by flash chromatography using silica gel column and 20 to 50% ethyl acetate in hexane as eluent to afford N-(6-hydroxyquinolin-2-yl)hex-5-ynamide (4) LCMS m/z 255.10 [M+1]⁺.

### Synthesis of (2R,3R,4S,5S,6R)-2-(2-(diethoxyphosphoryl)ethyl)-6-((2-(hex-5-ynamido)quinolin-6-yl)oxy)tetrahydro-2H-pyran-3,4,5-triyl triacetate (5)

To a solution of (2R,3R,4S,5S,6R)-2-(2-(diethoxyphosphoryl)ethyl)-6-(2,2,2-trichloro-1-iminoethoxy)tetrahydro-2H-pyran-3,4,5-triyl triacetate (4a, 1.0 eq.) in dry dichloromethane, N-(6-hydroxyquinolin-2-yl)hex-5-ynamide (4, 2.0 eq.) is added and resulting solution is cooled to −78° C. Boron trifluoride diethyl etherate (0.75 eq) is added slowly and reaction mixture is allowed to come at room temperature and stirred for 16 h. After completion of reaction, reaction quenched with saturated aqueous sodium bicarbonate solution and partitioned in between dichloromethane and aqueous phase. Aqueous layer re-extracted with dichloromethane, the combined organic layer is dried over anhydrous sodium sulfate, filtered, concentrated under reduce pressure, and purified by flash column chromatography using silica gel column to obtain ((2R,3R,4S,5S,6R)-2-(2-(diethoxyphosphoryl)ethyl)-6-((2-(hex-5-ynamido)quinolin-6-yl)oxy)tetrahydro-2H-pyran-3,4,5-triyl triacetate (5) LCMS m/z 677.24 [M+1]⁺.

### Synthesis of (2-((2R,3R,4S,5S,6R)-3,4,5-triacetoxy-6-((2-(hex-5-ynamido)quinolin-6-yl)oxy)tetrahydro-2H-pyran-2-yl)ethyl)phosphonic acid (6)

To a solution of ((2R,3R,4S,5S,6R)-2-(2-(diethoxyphosphoryl)ethyl)-6-((2-(hex-5-ynamido)quinolin-6-yl)oxy)tetrahydro-2H-pyran-3,4,5-triyl triacetate (5, 1.0 eq) in dichloromethane at 0° C. are added pyridine (15 eq) and bromotrimethylsilane (10 eq) and reaction mixture is stirred at room temperature for 4 h. LCMS showed consumption of starting material. Reaction mixture is cooled to 0° C. and quenched by addition of cold water. Dichloromethane layer is separated and aqueous layer re-extracted with dichloromethane. combined dichloromethane layer is dried over anhydrous sodium sulphate and concentrated under reduced pressure to afford (2-((2R,3R,4S,5S,6R)-3,4,5-triacetoxy-6-((2-(hex ynamido)quinolin-6-yl)oxy)tetrahydro-2H-pyran-2-yl)ethyl)phosphonic acid (6). LCMS m/z 621.18 [M+1]⁺.

### Synthesis of (2-((2R,3S,4S,5S,6R)-6-(4-ethynylphenoxy)-3,4,5-trihydroxytetrahydro-2H-pyran-2-yl)ethyl)phosphonic acid (Cpd. No. I-79)

To a solution of (2-((2R,3R,4S,5S,6R)-3,4,5-triacetoxy-6-((2-(hex-5-ynamido)quinolin-6-yl)oxy)tetrahydro-2H-pyran-2-yl)ethyl)phosphonic acid (6, 1.0 eq.) in methanol at 0° C. is added sodium methanolate (3.0 eq.) and reaction mixture is stirred at room temperature for 4 h. Reaction mixture cooled and quenched by addition of Dowex® 50W X8 hydrogen form up to neutral pH and filtered through sintered funnel. Filtrate obtained is concentrated under reduced pressure to get crude product. Crude product obtained is purified by reverse phase preparative HPLC to afford (2-((2R,3S,4S,5S,6R)-6-(4-ethynylphenoxy)-3,4,5-trihydroxytetrahydro-2H-pyran-2-yl)ethyl)phosphonic acid (Cpd. No. I-79). LCMS m/z 495.14 [M+1]⁺.

### Example 80: Synthesis of (2-((2R,3S,4S,5S,6R)-6-(4-(3-(hex-5-yn-1-yl)ureido)-2-hydroxyphenoxy)-3,4,5-trihydroxytetrahydro-2H-pyran-2-yl)ethyl)phosphonic acid (Cpd. No. I-80)

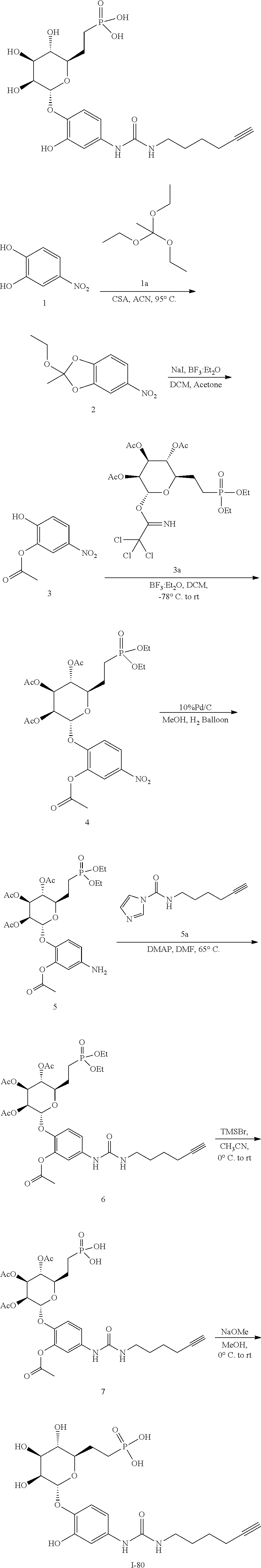

### Synthesis of 2-ethoxy-2-methyl-5-nitrobenzo[d][1,3]dioxole (2)

To a solution 4-nitrobenzene-1,2-diol (1, 2.0 g, 12.9 mmol) in acetonitrile (20.0 mL) were added camphor sulfonic acid (0.449 g, 0.019 mmol) and 1,1,1-triethoxyethane (23.8 mL, 129 mmol). The reaction mixture was stirred at 95° C. for 18 h. After completion (monitored by TLC), the reaction mixture was concentrated to get crude which was purified by column chromatography (100-200 mesh silica) using 0-10% ethyl acetate in hexane to afford 2-methoxy-2-methyl-5-nitro-2H-1,3-benzodioxole (2) as white solid. Yield: 1.0 g, 34.44%. LCMS m/z 226.07 [M+1]+.

### Synthesis of 2-hydroxy-5-nitrophenyl acetate (3)

To a solution of 2-ethoxy-2-methyl-5-nitrobenzo[d][1,3]dioxole (2, 1.00 g, 1.0 eq, 4.4 mmol) in dichloromethane (5 mL) at 0° C. is added an anhydrous solution of sodium iodide (1.97 g, 3 equiv, 13.2 mmol) in acetone (5.0 mL), and boron trifluoride etherate (0.72 mL, 1.33 eq., 5.85 mmol) under nitrogen. After 5 min at 0° C., water (20 mL) and dichloromethane (20 mL) are added. The layers are separated, and after back-extraction of the water layer, the combined dichloromethane layer is dried over anhydrous sodium sulfate, filtered and concentrated to afford 2-hydroxy-5-nitrophenyl acetate (3). LCMS m/z 198.09 [M+1]+.

### Synthesis of (2R,3S,4S,5R,6R)-2-(2-acetoxy-4-nitrophenoxy)-6-(2-(diethoxyphosphoryl)ethyl)tetrahydro-2H-pyran-3,4,5-triyl triacetate (4)

To a solution of [(2R,3R,4S,5S,6R)-4,5-diacetoxy-2-(2-diethoxyphosphorylethyl)-6-(2,2,2-trichloroethanimidoyl)oxy-tetrahydropyran-3-yl] acetate (3a, 1.0 g, 1.0 eq, 1.71 mmol) in dry dichloromethane (10 mL) with stirring under nitrogen. 2-hydroxy-5-nitrophenyl acetate (3, 0.33 g, 1.0 eq, 1.71 mmol) is added and the resulting clear solution is cooled to −78° C. with stirring under nitrogen. Boron trifluoride diethyl etherate (0.24 g, 1.0 eq, 1.71 mmol) is added slowly. The −78° C. cold bath is removed and replaced with a 0° C. cold bath. The reaction mixture is stirred at 0° C. for 2 h. The reaction mixture is partitioned between dichloromethane and saturated aqueous sodium bicarbonate. The water layer is extracted again with dichloromethane. The combined organics is dried over anhydrous sodium sulfate, filtered, and concentrated on a rotary evaporator, and purified via silica gel chromatography to afford (2R,3S,4S,5R,6R)-2-(2-acetoxy-4-nitrophenoxy)-6-(2-(diethoxyphosphoryl)ethyl)tetrahydro-2H-pyran-3,4,5-triyl triacetate (4). LCMS m/z 620.17 [M+1]+.

### Synthesis of (2R,3S,4S,5R,6R)-2-(2-acetoxy-4-aminophenoxy)-6-(2-(diethoxyphosphoryl)ethyl)tetrahydro-2H-pyran-3,4,5-triyl triacetate (5)

To a solution of (2R,3S,4S,5R,6R)-2-(2-acetoxy-4-nitrophenoxy)-6-(2-(diethoxyphosphoryl)ethyl)tetrahydro-2H-pyran-3,4,5-triyl triacetate (4, 0.50 g, 1.0 eq, 0.807 mmol) in methanol (5.0 mL) is added 10% palladium on carbon (0.20 g). The reaction mixture is stirred at room temperature for 3 h under hydrogen atmosphere. After completion, the reaction mixture is filtered through syringe filter, filtrate is concentrated and dried to afford (2R,3S,4S,5R,6R)-2-(2-acetoxy-4-aminophenoxy)-6-(2-(diethoxyphosphoryl)ethyl)tetrahydro-2H-pyran-3,4,5-triyl triacetate (5). LCMS m/z 590.14 [M+1]+.

### Synthesis (2R,3S,4S,5R,6R)-2-(2-acetoxy-4-(3-(hex-5-yn-1-yl)ureido)phenoxy)-6-(2-(diethoxyphosphoryl)ethyl)tetrahydro-2H-pyran-3,4,5-triyl triacetate (6)

To a solution of (2R,3S,4S,5R,6R)-2-(2-acetoxy-4-aminophenoxy)-6-(2-(diethoxyphosphoryl)ethyl)tetrahydro-2H-pyran-3,4,5-triyl triacetate (5, 0.50 g, 1.0 eq, 0.84 mmol) in N,N-dimethyl formamide (5.0 mL) is added N-(hex-5-yn-1-yl)-1H-imidazole-1-carboxamide (5a, 0.192 g, 1.2 eq, 1.008 mmol) and 4-dimethylaminopyridine (0.102 g, 1.0 eq, 0.84 mmol). The reaction mixture is stirred at 60° C. for 24 h. After completion, the reaction mixture is diluted with water and extracted with ethyl acetate. The organic layer is dried over anhydrous sodium sulfate, filtered and concentrated under reduced pressure to get crude product. The crude is purified by flash chromatography (silica mesh: 100-200) to afford (2R,3S,4S,5R,6R)-2-(2-acetoxy-4-(3-(hex-5-yn-1-yl)ureido)phenoxy)-6-(2-(diethoxyphosphoryl)ethyl)tetrahydro-2H-pyran-3,4,5-triyl triacetate (6). LCMS m/z 713.16 [M+1]⁺.

### Synthesis of (2-((2R,3R,4S,5S,6R)-3,4,5-triacetoxy-6-(2-acetoxy-4-(3-(hex-5-yn-1-yl)ureido)phenoxy)tetrahydro-2H-pyran-2-yl)ethyl)phosphonic acid (7)

To a solution of (2R,3S,4S,5R,6R)-2-(2-acetoxy-4-(3-(hex-5-yn-1-yl)ureido)phenoxy)-6-(2-(diethoxyphosphoryl)ethyl)tetrahydro-2H-pyran-3,4,5-triyl triacetate (6, 0.50 g, 1.0 eq, 0.702 mmol) in acetonitrile (5.0 mL) is added bromotrimethylsilane (0.46 mL, 5.0 eq, 3.51 mmol) at 0° C. The reaction mixture is stirred at room temperature for 5 h. After completion, the reaction mixture is concentrated under reduced pressure to obtain sticky mass which is triturated with diethyl ether to obtain (2-((2R,3R,4S,5S,6R)-3,4,5-triacetoxy-6-(2-acetoxy-4-(3-(hex-5-yn-1-yl)ureido)phenoxy)tetrahydro-2H-pyran-2-yl)ethyl)phosphonic acid (7) as crude compound, which is used as such for next step without further purification. LCMS m/z 657.20 [M+1]+.

### Synthesis of (2-((2R,3S,4S,5S,6R)-6-(4-(3-(hex-5-yn-1-yl)ureido)-2-hydroxyphenoxy)-3,4,5-trihydroxytetrahydro-2H-pyran-2-yl)ethyl)phosphonic acid (Cpd. No. I-80)

To a solution of (2-((2R,3R,4S,5S,6R)-3,4,5-triacetoxy-6-(2-acetoxy-4-(3-(hex-5-yn-1-yl)ureido)phenoxy)tetrahydro-2H-pyran-2-yl)ethyl)phosphonic acid (7, 0.50 g, 1.0 eq, 0.76 mmol) in methanol (5.0 mL) is added sodium methanolate (0.49 mL, 10.0 eq, 2.28 mmol) at 0° C. The reaction mixture is stirred at 0° C. to room temperature for 3 h. After completion, the reaction mixture is neutralized with Dowex 50WX8 hydrogen form, filtered and concentrated under reduced pressure to obtain crude. The crude is purified by reverse phase preparative HPLC to afford (2-((2R,3S,4S,5S,6R)-6-(4-(3-(hex-5-yn-1-yl)ureido)-2-hydroxyphenoxy)-3,4,5-trihydroxytetrahydro-2H-pyran-2-yl)ethyl)phosphonic acid (Cpd. No. I-80). LCMS m/z 489.07 [M+1]⁺.

### Example 81: Synthesis of Compound I-81

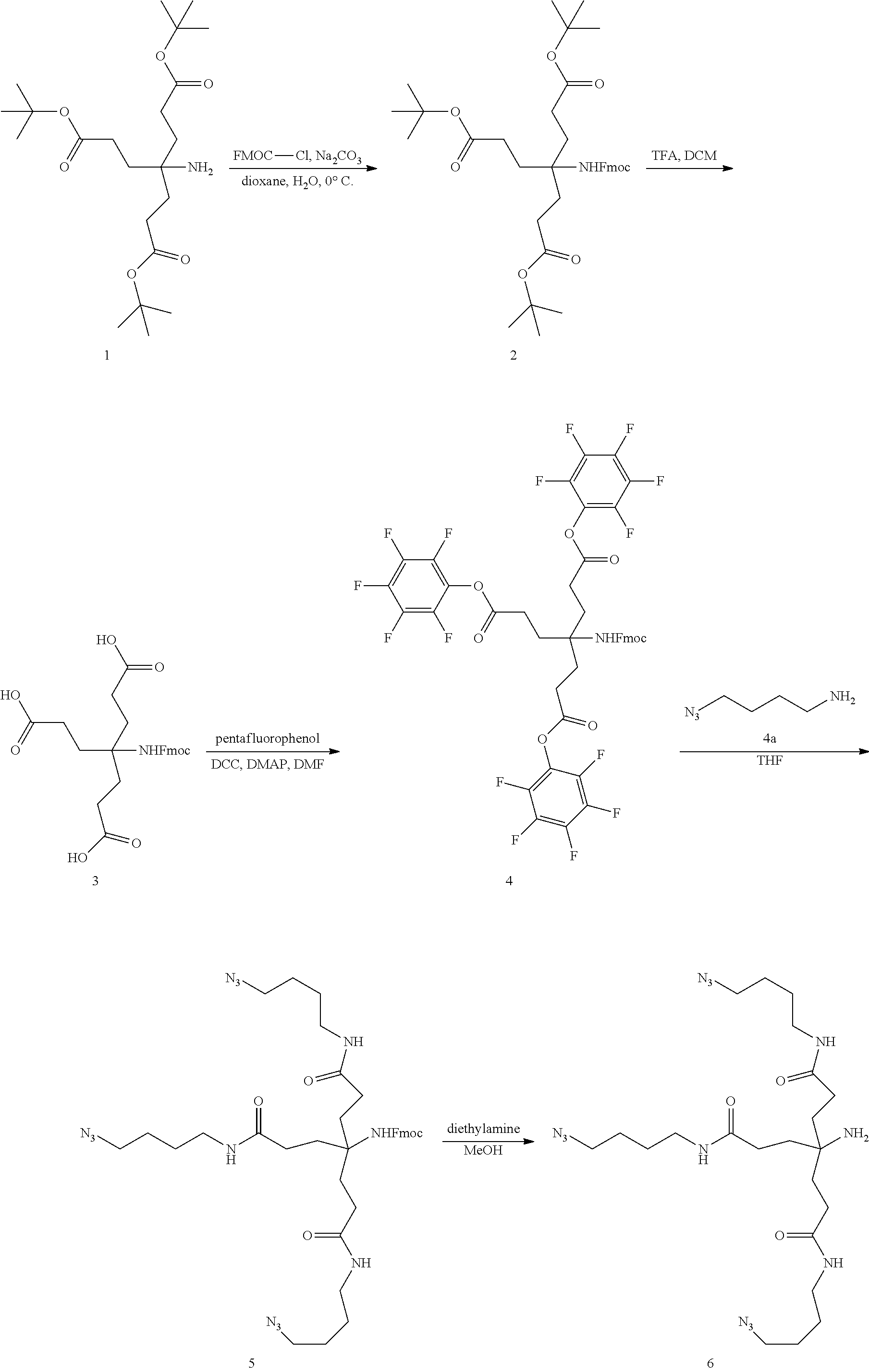

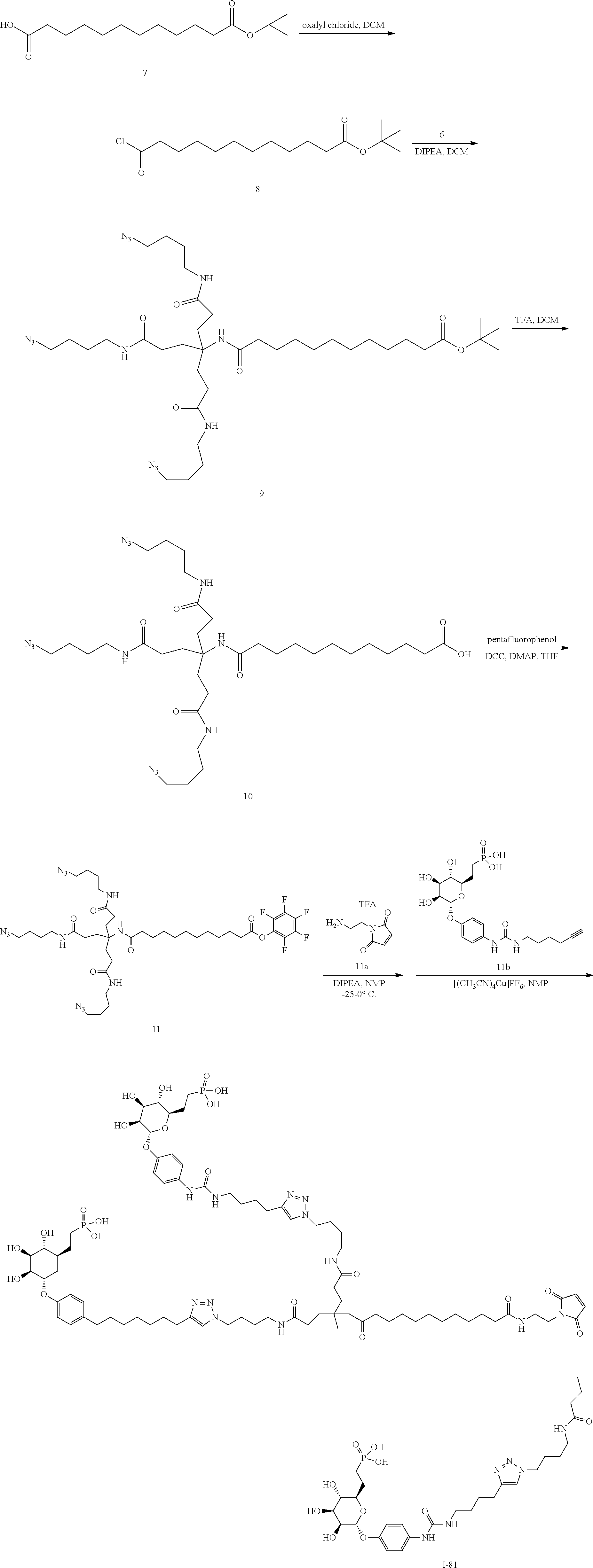

### Synthesis of di-tert-butyl 4-((((9H-fluoren-9-yl)methoxy)carbonyl)amino)-4-(3-(tert-butoxy)-3-oxopropyl)heptanedioate (2)

To a stirred mixture of di-tert-butyl 4-amino-4-(3-(tert-butoxy)-3-oxopropyl)heptanedioate (1, 1.00 eq, 1.01 g, 2.43 mmol) in 1,4-dioxane (10 mL) at 0° C. was added 1 M sodium carbonate in water (1.50 eq, 3.6 mL, 3.65 mmol) and then a solution of FMOC-Cl (1.20 eq, 755 mg, 2.92 mmol) in 1,4-dioxane (4 mL). The cold bath was removed and the resulting mixture was stirred vigorously at room temperature for 2 h. The reaction mixture was partitioned between ethyl acetate and brine. The organics were dried over magnesium sulfate, filtered, concentrated on a rotary evaporator, and purified via silica gel chromatography (0-30% ethyl acetate in hexanes) to afford di-tert-butyl 4-((((9H-fluoren-9-yl)methoxy)carbonyl)amino)-4-(3-(tert-butoxy)-3-oxopropyl)heptanedioate (2) as a white foam-solid. Yield: 1.50 g, 97%; LCMS m/z 660.6 [M+Na]+; ¹H NMR (300 MHz, Chloroform-c) δ 7.76 (d, J=7.4 Hz, 2H), 7.59 (d, J=7.4 Hz, 2H), 7.40 (t, J=7.5 Hz, 2H), 7.31 (t, J=7.4 Hz, 2H), 5.01 (s, 1H), 4.36 (d, J=6.2 Hz, 2H), 4.18 (t, J=6.5 Hz, 1H), 2.25-2.12 (m, 6H), 1.98-1.83 (m, 6H), 1.43 (s, 27H).

### Synthesis of 4-((((9H-fluoren-9-yl)methoxy)carbonyl)amino)-4-(2-carboxyethyl)heptanedioic acid (3)

To a stirred solution of di-tert-butyl 4-((((9H-fluoren yl)methoxy)carbonyl)amino)-4-(3-(tert-butoxy)-3-oxopropyl)heptanedioate (2, 1.00 eq, 1.50 g, 2.35 mmol) in DCM (10 mL) at 0° C. was added water (0.5 mL) and then TFA (3 mL). The resulting mixture was allowed to warm to room temperature and then stirred at room temperature for 18 h. More TFA (2 mL) was added and stirring at room temperature was continued for another 26 h. Volatiles were removed on a rotary evaporator. The residue was concentrated to dryness twice from dry toluene and then dried under high vacuum to afford 4-((((9H-fluoren-9-yl)methoxy)carbonyl)amino)-4-(2-carboxyethyl)heptanedioic acid (3) as a white solid. Yield: 1.19 g. LCMS 470.4 m/z [M+1]+; ¹H NMR (300 MHz, DMSO-d₆ with D₂O) δ 7.86 (d, J=7.5 Hz, 2H), 7.68 (d, J=7.5 Hz, 2H), 7.39 (t, J=7.4 Hz, 2H), 7.30 (t, J=7.9 Hz, 2H), 4.28-4.11 (m, 3H), 2.19-2.00 (m, 6H), 1.87-1.66 (m, 6H).

### Synthesis of bis(perfluorophenyl) 4-((((9H-fluoren-9-yl)methoxy)carbonyl)amino)-4-(3-oxo-3-(perfluorophenoxy)propyl)heptanedioate (4)

4-((((9H-fluoren-9-yl)methoxy)carbonyl)amino)-4-(2-carboxyethyl)heptanedioic acid (3, 1.00 eq, 549 mg, 1.17 mmol), 4-dimethylaminopyridine (0.0200 eq, 2.9 mg, 0.0234 mmol), N,N′-dicyclohexylcarbodiimide (3.30 eq, 796 mg, 3.86 mmol), pentafluorophenol (3.50 eq, 753 mg, 4.09 mmol), and DMF (2.5 mL) were combined in a scintillation vial with a stirbar, capped, and stirred at room temperature for 4 h. More N,N′-dicyclohexylcarbodiimide (482 mg, 2.34 mmol) and pentafluorophenol (430 mg, 2.34 mmol) in DMF (1 mL) was added and the resulting mixture was capped and stirred at room temperature for 2 h. The reaction mixture was diluted with diethyl ether and filtered. The filtrate was washed three times with brine, dried over magnesium sulfate, filtered, concentrated on a rotary evaporator, and purified via silica gel chromatography (0-50% ethyl acetate in hexanes) to afford bis(perfluorophenyl) 4-((((9H-fluoren-9-yl)methoxy)carbonyl)amino)-4-(3-oxo-3-(perfluorophenoxy)propyl)heptanedioate (4) and pentafluorophenol as a light yellow oil. Yield: 1.54 g. This material was taken on to the next step without further purification.

### Synthesis of (9H-fluoren-9-yl)methyl (1,7-bis((4-azidobutyl)amino)-4-(3-((4-azidobutyl)amino)-3-oxopropyl)-1,7-dioxoheptan-4-yl)carbamate (5)

4-Azidobutan-1-amine (4a, 0.5 M in mTBE) (4.00 eq, 8.7 mL, 4.34 mmol) was added to a stirred solution of bis(perfluorophenyl) 4-((((9H-fluoren-9-yl)methoxy)carbonyl)amino)-4-(3-oxo-3-(perfluorophenoxy)propyl)heptanedioate (4, 1.00 eq, 1.50 g, 1.09 mmol) in THF (10 mL) at room temperature. The resulting clear solution was capped and stirred at room temperature for 2 h. Most of the volatiles were removed on a rotary evaporator at room temperature. The residue was loaded onto a silica gel loading column with dichloromethane and purified via silica gel chromatography (0-100% ethyl acetate in dichloromethane) then (0-10% methanol in dichloromethane) to afford (9H-fluoren-9-yl)methyl (1,7-bis((4-azidobutyl)amino)-4-(3-((4-azidobutyl)amino)-3-oxopropyl)-1,7-dioxoheptan-4-yl)carbamate (5) as a colorless waxy solid. Yield: 624 mg, 76%; LCMS m/z 758.6 [M+1]+; ¹H NMR (300 MHz, Chloroform-d) δ 7.77 (d, J=7.5 Hz, 2H), 7.60 (d, J=7.4 Hz, 2H), 7.41 (t, J=7.4 Hz, 2H), 7.31 (t, J=7.4 Hz, 2H), 6.08 (bs, 3H), 5.67 (bs, 1H), 4.37 (d, J=7.0 Hz, 2H), 4.18 (t, J=6.7 Hz, 1H), 3.34-3.13 (m, 12H), 2.24-2.09 (m, 6H), 2.04-1.85 (m, 6H), 1.66-1.47 (m, 12H).

### Synthesis of 4-amino-N1,N7-bis(4-azidobutyl)-4-(3-((4-azidobutyl)amino)-3-oxopropyl)heptanediamide (6)

Diethylamine (20.0 eq, 1.7 mL, 16.3 mmol) was added to a stirred solution of (9H-fluoren-9-yl)methyl (1,7-bis((4-azidobutyl)amino)-4-(3-((4-azidobutyl)amino)-3-oxopropyl)-1,7-dioxoheptan-4-yl)carbamate (5, 1.00 eq, 619 mg, 0.817 mmol) in methanol (8 mL). The resulting clear solution was capped and stirred at room temperature for 16 h. Volatiles were removed on a rotary evaporator. Methanol (10 mL) was added and volatiles were removed on a rotary evaporator again. This was repeated again to drive off diethylamine. The residue was taken up in methanol and loaded onto a 5 g Strata X-C ion exchange column from Phenomenex. The column was eluted sequentially with acetonitrile, methanol, and then 5% ammonium hydroxide in methanol. Fractions containing the desired product were combined, concentrated on a rotary evaportor and dried under high vacuum to afford 4-amino-N1,N7-bis(4-azidobutyl)-4-(3-((4-azidobutyl)amino)-3-oxopropyl)heptanediamide (6) at 90% purity as a yellow oil. Yield: 483 mg, 99%; LCMS m/z 536.8 [M+1]⁺; ¹H NMR (300 MHz, Chloroform-d) δ 6.33 (t, J=5.8 Hz, 3H), 3.48 (s, 2H), 3.36-3.17 (m, 12H), 2.33-2.12 (m, 6H), 1.74-1.51 (m, 18H).

### Synthesis of tert-butyl 12-chloro-12-oxododecanoate (8)

To a stirred solution of 12-(tert-butoxy)-12-oxododecanoic acid (7, 1.00 eq, 975 mg, 3.40 mmol) in DCM (7 mL) at room temperature under nitrogen was added DMF (5 microliters) and then oxalyl chloride (2 M in methylene chloride) (1.15 eq, 2.0 mL, 3.91 mmol). The resulting clear solution was stirred at room temperature under nitrogen for 1 h. Vigorous bubbling was observed. More oxalyl chloride (2 M in methylene chloride) (1.0 mL, 2.0 mmol) was added and the resulting mixture was stirred at room temperature under nitrogen for 30 min and then volatiles were removed on a rotary evaporator. The residue was dried under high vacuum to afford a yellow oil which was used in the next step without purification.

### Synthesis of tert-butyl 12-((1,7-bis((4-azidobutyl)amino)-4-(3-((4-azidobutyl)amino)-3-oxopropyl)-1,7-dioxoheptan-4-yl)amino)-12-oxododecanoate (9)

A solution of 4-amino-N1,N7-bis(4-azidobutyl)-4-(3-((4-azidobutyl)amino) oxopropyl)heptanediamide (6, 1.00 eq, 707 mg, 1.19 mmol) and N,N-diisopropylethylamine (6.00 eq, 1.2 mL, 7.13 mmol) in DCM (4 mL) was added to a stirred solution of tert-butyl 12-chloro-12-oxododecanoate (8, 3.00 eq, 1.09 g, 3.56 mmol) in DCM (4 mL) at 0° C. under nitrogen. The resulting yellow solution was capped and stirred at room temperature for 30 min. Volatiles were removed on a rotary evaporator. The residue was taken up in acetic acid, and purified via reverse-phase flash chromatography (10-100% acetonitrile in water with 0.1% formic acid). Fractions containing the desired product were combined and concentrated at 30° C. on a rotary evaporator and the residue was dried under high vacuum to afford tert-butyl 12-((1,7-bis((4-azidobutyl)amino)-4-(3-((4-azidobutyl)amino)-3-oxopropyl)-1,7-dioxoheptan-4-yl)amino)-12-oxododecanoate (9) as a colorless oil. Yield: 596 mg, 62%; LCMS m/z 804.8 [M+1]⁺.

### Synthesis of 12-((1,7-bis((4-azidobutyl)amino)-4-(3-((4-azidobutyl)amino)-3-oxopropyl)-1,7-dioxoheptan-4-yl)amino)-12-oxododecanoic acid (10)

tert-Butyl 12-((1,7-bis((4-azidobutyl)amino)-4-(3-((4-azidobutyl)amino)-3-oxopropyl)-1,7-dioxoheptan-4-yl)amino)-12-oxododecanoate (9, 1.00 eq, 592 mg, 0.736 mmol) was dissolved with stirring in DCM (4 mL) and then cooled to 0° C. Water (2 drops) was added and then TFA (2 mL) was added slowly down the side of the flask. The cold bath was removed and the resulting clear solution was stirred at room temperature for 1 h 20 min. Volatiles were removed on a rotary evaporator. The residue was taken up in acetic acid and purified via reverse-phase flash chromatography (10-100% acetonitrile in water with 0.1% formic acid). Fractions containing the desired product were combined, concentrated on a rotary evaporator, and dried under high vacuum to afford 12-((1,7-bis((4-azidobutyl)amino)-4-(3-((4-azidobutyl)amino)-3-oxopropyl)-1,7-dioxoheptan-4-yl)amino)-12-oxododecanoic acid (10) as a colorless oil. Yield: 440 mg, 80%; LCMS m/z 748.7 [M+1]⁺; ¹H NMR (300 MHz, Chloroform-d) δ 7.13 (bs, 1H), 6.68 (bs, 3H), 3.37-3.16 (m, 12H), 2.38-2.20 (m, 8H), 2.15 (t, J=7.4 Hz, 2H), 2.08-1.96 (m, 6H), 1.72-1.49 (m, 16H), 1.41-1.18 (m, 12H).

### Synthesis of perfluorophenyl 12-((1,7-bis((4-azidobutyl)amino)-4-(3-((4-azidobutyl)amino)-3-oxopropyl)-1,7-dioxoheptan-4-yl)amino)-12-oxododecanoate (11)

To a stirred solution of 12-((1,7-bis((4-azidobutyl)amino)-4-(3-((4-azidobutyl)amino)-3-oxopropyl)-1,7-dioxoheptan-4-yl)amino)-12-oxododecanoic acid (10, 1.00 eq, 436 mg, 0.583 mmol) in THF (2.5 mL) was added sequentially: N,N′dicyclohexylcarbodiimide (1.50 eq, 180 mg, 0.874 mmol), a solution of 2,3,4,5,6-pentafluorophenol (1.50 eq, 161 mg, 0.874 mmol) in THF (1 mL), and then 4-dimethylaminopyridine (0.0200 eq, 1.4 mg, 0.0117 mmol). The resulting mixture was capped and stirred at room temperature for 1.5 h. More N,N′dicyclohexylcarbodiimide (107 mg, 0.52 mmol) was added and stirring at room temperature was continued for another 21.5 h. The reaction mixture was diluted with diethyl ether and filtered. The filtrate was concentrated on a rotary evaporator. The residue was taken up in acetic acid and purified via reverse-phase flash chromatography (10-100% acetonitrile in water with 0.1% formic acid). Fractions containing the desired product were combined and lyophilized to dryness to afford perfluorophenyl 12-((1,7-bis((4-azidobutyl)amino)-4-(3-((4-azidobutyl)amino)-3-oxopropyl)-1,7-dioxoheptan-4-yl)amino)-12-oxododecanoate (11) as a colorless wax. Yield: 431 mg, 81%; LCMS m/z 914.7 [M+1]+; ¹H NMR (300 MHz, Chloroform-d) δ 7.18 (bs, 1H), 6.14 (bs, 3H), 3.38-3.14 (m, 12H), 2.66 (t, J=7.4 Hz, 2H), 2.30-1.92 (m, 14H), 1.83-1.68 (m, 2H), 1.68-1.49 (m, 14H), 1.45-1.20 (m, 12H).

### Synthesis of Cpd. No. I-81

A solution of 1-(2-aminoethyl)-1H-pyrrole-2,5-dione TFA salt (11a, 1.00 eq, 6.8 mg, 0.0268 mmol) and N,N-diisopropylethylamine (1.30 eq, 0.0061 mL, 0.0348 mmol) in NMP (0.3 mL) was added to a stirred solution of perfluorophenyl 12-((1,7-bis((4-azidobutyl)amino)-4-(3-((4-azidobutyl)amino)-3-oxopropyl)-1,7-dioxoheptan-4-yl)amino)-12-oxododecanoate (11, 1.00 eq, 24.5 mg, 0.0268 mmol) in DMF (0.3 mL) at −25° C. The resulting mixture was capped, stirred, and allowed to slowly warm to room temperature over 30 min. After warming to room temperature (2-((2R,3S,4S,5S,6R)-6-(4-(3-(hex-5-yn-1-yl)ureido)phenoxy)-3,4,5-trihydroxytetrahydro-2H-pyran-2-yl)ethyl)phosphonic acid (11b, 3.20 eq, 40.5 mg, 0.0858 mmol) was added. The resulting solution was stirred at room temperature for 3 min and then tetrakis(acetonitrile)copper(I) hexafluorophosphate (7.50 eq, 74.9 mg, 0.201 mmol) was added. The resulting light yellow solution was capped and stirred at room temperature for 25 min. Slowly turned more green-colored. The reaction mixture was diluted with a mixture of NMP and acetic acid, filtered, and purified via preparatory HPLC (10-50% acetonitrile in water with 0.1% TFA). Fractions containing the desired product were combined and lyophilized to dryness to afford (Cpd. No. I-81) as a white solid. Yield: 14.1 mg, 23%; ¹H NMR (300 MHz, DMSO-d₆ with D₂O) δ 7.77 (s, 3H), 7.29-7.17 (m, 6H), 6.94-6.82 (m, 8H), 5.24 (s, 3H), 4.24 (t, J=6.8 Hz, 6H), 3.84-3.77 (m, 3H), 3.65-3.54 (m, 3H), 3.45-2.88 (m, 20H), 2.63-2.54 (m, 6H), 2.05-1.03 (m, 70H).

### Example 82: Synthesis of Compound I-82

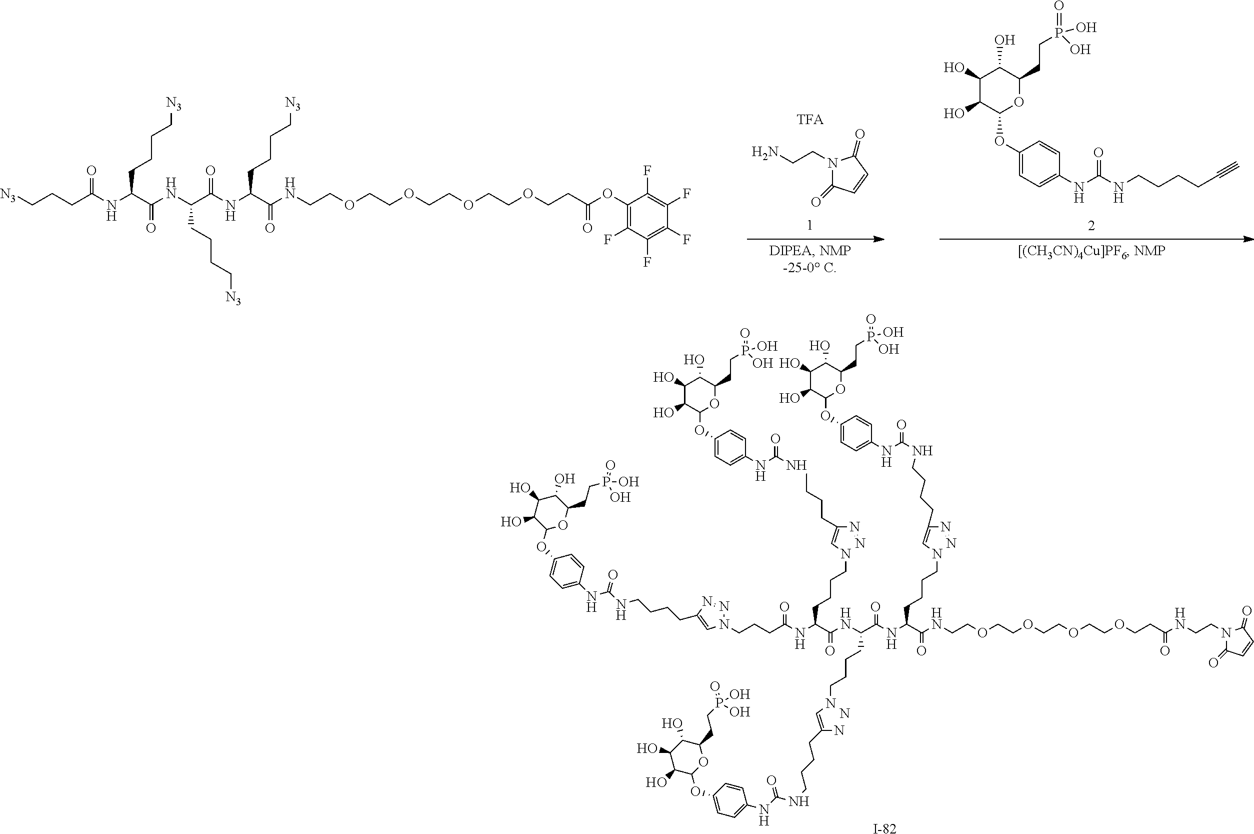

A solution of 1-(2-aminoethyl)-1H-pyrrole-2,5-dione TFA salt (1, 1.10 eq, 5.6 mg, 0.0221 mmol) and N,N-diisopropylethylamine (1.30 eq, 0.0046 mL, 0.0261 mmol) in NMP (0.4 mL) was added to a stirred solution of perfluorophenyl (18S,21S,24S)-29-azido-18,21,24-tris(4-azidobutyl)-17,20,23,26-tetraoxo-4,7,10,13-tetraoxa-16,19,22,25-tetraazanonacosanoate (1.00 eq, 20.2 mg, 0.0201 mmol) in DMF (0.6 mL) at −25° C. The resulting mixture was capped, stirred, and allowed to slowly warm to 0° C. over 30 min. The cold bath was removed and (2-((2R,3S,4S,5S,6R)-6-(4-(3-(hex-5-yn-1-yl)ureido)phenoxy)-3,4,5-trihydroxytetrahydro-2H-pyran-2-yl)ethyl)phosphonic acid (2, 5.00 eq, 47.5 mg, 0.101 mmol) was added and the resulting mixture was capped and stirred for 3 min before tetrakis(acetonitrile)copper(I) hexafluorophosphate (20.0 eq, 150 mg, 0.402 mmol) was added. The resulting light yellow solution was capped and stirred at room temperature for 25 min. (Slowly turned more green-colored). The reaction mixture was diluted with a mixture of NMP, acetic acid, and TFA, filtered, and purified via preparatory HPLC (5-35 acetonitrile in water with 0.1% TFA). Fractions containing the desired product were combined and lyophilized to dryness to afford (Cpd. No. I-82) as a white solid. Yield: 22.8 mg, 40%; ¹H NMR (300 MHz, DMSO-d₆ with D₂O) δ 7.81-7.70 (m, 4H), 7.29-7.17 (m, 8H), 6.94-6.82 (m, 10H), 5.24 (s, 4H), 4.31-4.05 (m, 11H), 3.84-3.75 (m, 4H), 3.67-3.55 (m, 4H), 3.54-2.94 (m, 35H), 2.59-2.53 (m, 8H), 2.19 (t, J=6.0 Hz, 2H), 2.15-2.04 (m, 2H), 2.04-1.81 (m, 6H), 1.79-1.04 (m, 46H).

### Example 83: Synthesis of (2-((2R,3S,4S,5S,6R)-6-(4-(3-(4-(1-(1-bromo oxo-6,9,12-trioxa-3-azatetradecan-14-yl)-1H-1,2,3-triazol-4-yl)butyl)ureido)phenoxy)-3,4,5-trihydroxytetrahydro-2H-pyran-2-yl)ethyl)phosphonic acid (Compound I-83)

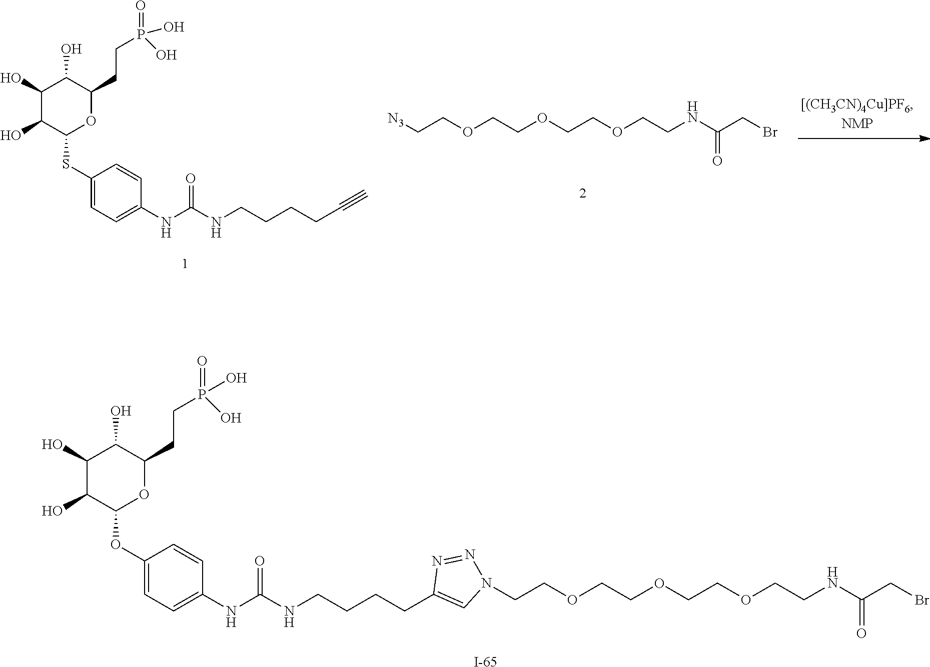

To (2-((2R,3S,4S,5S,6R)-6-(4-(3-(hex-5-yn-1-yl)ureido)phenoxy)-3,4,5-trihydroxytetrahydro-2H-pyran-2-yl)ethyl)phosphonic acid (1, 1.00 eq, 25.0 mg, 0.0529 mmol) in a 1 dram vial with a stirbar was added a solution of N-(2-(2-(2-(2-azidoethoxy)ethoxy)ethoxy)ethyl)-2-bromoacetamide (2, 1.15 eq, 20.6 mg, 0.0609 mmol) in NMP (0.4 mL) followed by tetrakis(acetonitrile)copper(I) hexafluorophosphate (2.50 eq, 49.3 mg, 0.132 mmol). The resulting clear light green solution was capped and stirred at room temperature for 20 min. The reaction mixture was diluted with a mixture of NMP, ethanol, and acetic acid, filtered, and purified via preparatory HPLC (10-35% acetonitrile in water with 0.1% TFA). Fractions containing the desired product were combined and lyophilized to dryness to afford (2-((2R,3S,4S,5S,6R)-6-(4-(3-(4-(1-(1-bromo-2-oxo-6,9,12-trioxa-3-azatetradecan-14-yl)-1H-1,2,3-triazol-4-yl)butyl)ureido)phenoxy)-3,4,5-trihydroxytetrahydro-2H-pyran-2-yl)ethyl)phosphonic acid (Cpd. No. I-83) as a white solid. Yield: 26.9 mg, 62.6%; LCMS m/z 813.4 [M+1]⁺; ¹H NMR (300 MHz, DMSO-d₆ with D₂O) δ 7.78 (s, 1H), 7.24 (d, J=7.7 Hz, 2H), 6.90 (d, J=7.7 Hz, 2H), 5.29-5.21 (m, 1H), 4.50-4.38 (m, 2H), 3.84-3.74 (m, 3H), 3.64-3.57 (m, 1H), 3.55-2.96 (m, 18H), 2.60 (t, J=7.6 Hz, 2H), 1.98-1.84 (m, 1H), 1.63-1.37 (m, 5H), 1.30-1.09 (m, 2H).

### Example 84: Synthesis of (2-((2R,3S,4S,5S,6R)-6-(4-(3-(4-(1-(19-(5-cyano-6-(methylsulfonyl)pyridin-2-yl)-15-oxo-3,6,9,12-tetraoxa-16-azanonadecyl)-1H-1,2,3-triazol-4-yl)butyl)ureido)phenoxy)-3,4,5-trihydroxytetrahydro-2H-pyran yl)ethyl)phosphonic acid (Cpd. No. I-84)

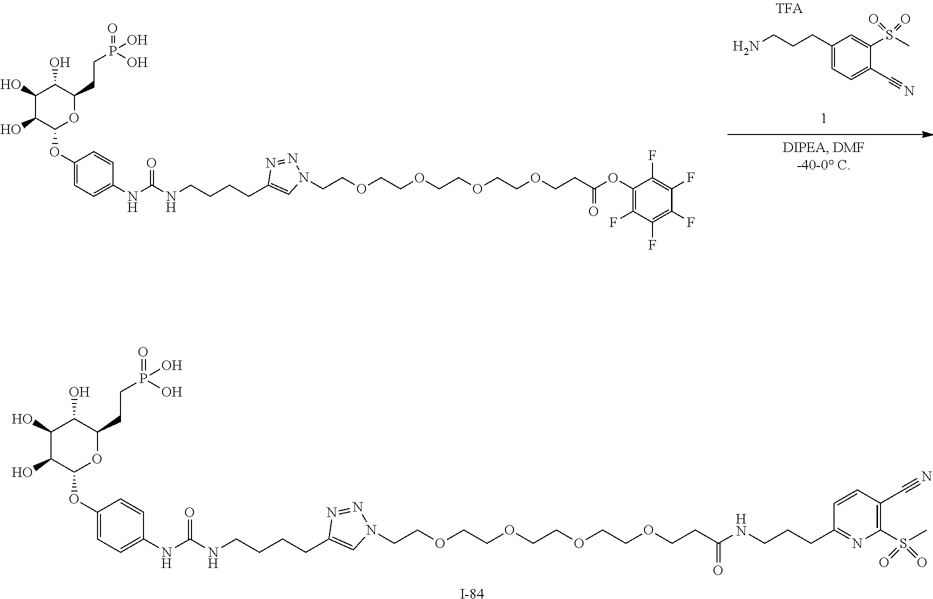

A solution of 4-(3-aminopropyl)-2-(methylsulfonyl)benzonitrile TFA salt (1, 1.10 eq, 12.5 mg, 0.0355 mmol) and N,N-diisopropylethylamine (13.0 eq, 0.073 mL, 0.419 mmol) in DMF (0.5 mL) was added to a solution of (2-((2R,3S,4S,5S,6R)-3,4,5-trihydroxy-6-(4-(3-(4-(1-(15-oxo-15-(perfluorophenoxy)-3,6,9,12-tetraoxapentadecyl)-1H-1,2,3-triazol-4-yl)butyl)ureido)phenoxy)tetrahydro-2H-pyran-2-yl)ethyl)phosphonic acid (1.00 eq, 30.0 mg, 0.0323 mmol) in DMF (0.5 mL) at −40° C. and the reaction was allowed to slowly warm to 0° C. over 20 min. The reaction mixture was diluted with acetic acid (0.3 mL), filtered, and purified via preparatory HPLC (10-30% acetonitrile in water with 0.1% TFA). Fractions containing the desired product were combined and lyophilized to dryness to afford (Cpd. No. I-84) (24 mg, 0.025 mmol, 77% yield) as a white solid. LCMS m/z 985.6 [M+1]⁺; ¹H NMR (300 MHz, DMSO-d6+D₂O) δ 8.43-8.36 (m, 1H), 7.76 (s, 1H), 7.75-7.68 (m, 1H), 7.23 (d, J=7.0 Hz, 2H), 6.89 (d, J=9.0 Hz, 2H), 5.28-5.20 (m, 1H), 4.47-4.37 (m, 2H), 3.84-3.78 (m, 1H), 3.78-3.69 (m, 2H), 3.65-3.50 (m, 3H), 3.48-3.27 (m, 17H), 3.13-2.99 (m, 5H), 2.87 (t, J=7.5 Hz, 2H), 2.59 (t, J=7.4 Hz, 2H), 2.26 (t, J=6.2 Hz, 2H), 1.98-1.76 (m, 3H), 1.62-1.37 (m, 5H), 1.28-1.15 (m, 1H).

### Example 85: Synthesis of (2-((2R,3S,4S,5S,6R)-3,4,5-trihydroxy-6-(4-((6-(1-(15-oxo-15-(perfluorophenoxy)-3,6,9,12-tetraoxapentadecyl)-1H-1,2,3-triazol-4-yl)hexyl)oxy)phenoxy)tetrahydro-2H-pyran-2-yl)ethyl)phosphonic acid (Cpd. No. I-85)

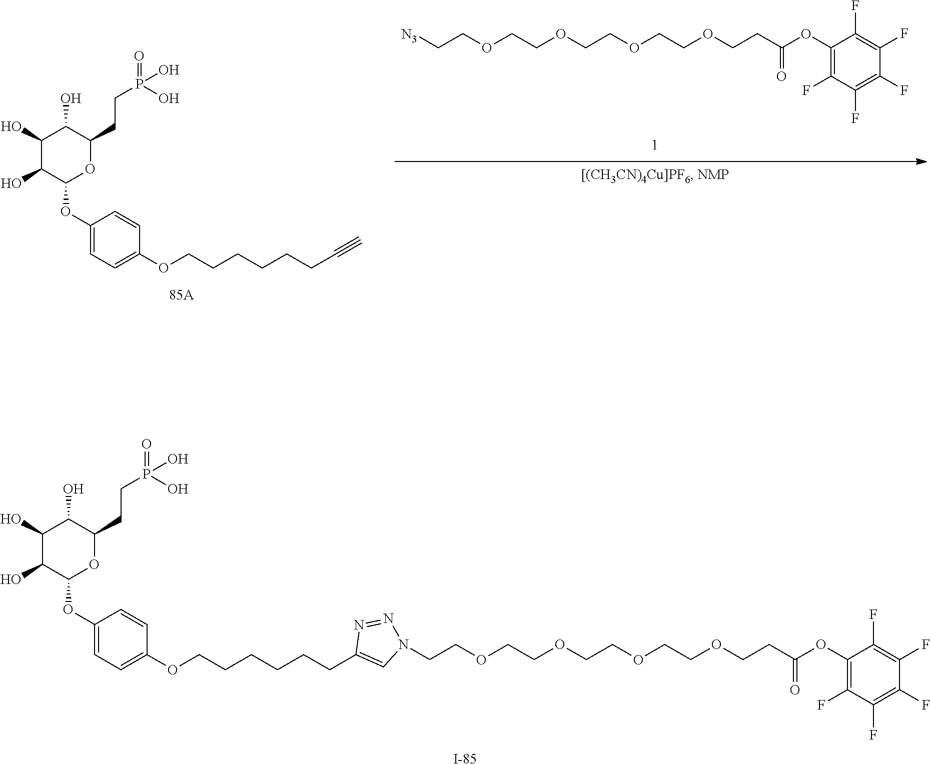

To (2-((2R,3S,4S,5S,6R)-3,4,5-trihydroxy-6-(4-(oct-7-yn-1-yloxy)phenoxy)tetrahydro-2H-pyran-2-yl)ethyl)phosphonic acid (85A, 1.00 eq, 36.0 mg, 0.0786 mmol) in a 1 dram vial with a stirbar was added a solution of perfluorophenyl 1-azido-3,6,9,12-tetraoxapentadecan-15-oate (1, 1.20 eq, 43.1 mg, 0.0943 mmol) in NMP (0.5 mL) followed by tetrakis(acetonitrile)copper(I) hexafluorophosphate (2.50 eq, 73.3 mg, 0.197 mmol). The resulting clear yellow solution was capped and stirred at room temperature for 20 minutes (slowly turned green colored). The reaction was diluted with a mixture of NMP, ethanol, and acetic acid, filtered, and purified via preparatory HPLC (15-60% acetonitrile in water with 0.1% TFA). Fractions containing the desired product were combined and lyophilized to dryness to afford (2-((2R,3S,4S,5S,6R)-3,4,5-trihydroxy-6-(4-((6-(1-(15-oxo-15-(perfluorophenoxy)-3,6,9,12-tetraoxapentadecyl)-1H-1,2,3-triazol-4-yl)hexyl)oxy)phenoxy)tetrahydro-2H-pyran-2-yl)ethyl)phosphonic acid (Cpd. No. I-85) as a white solid. Yield: 39.8 mg, 55%; LCMS m/z 916.5 [M+1]⁺; ¹H NMR (300 MHz, DMSO-d₆ with D₂O) δ 7.75 (s, 1H), 6.92 (d, J=8.1 Hz, 2H), 6.80 (d, J=8.5 Hz, 2H), 5.19 (s, 1H), 4.41 (t, J=4.8 Hz, 2H), 3.85-3.67 (m, 7H), 3.64-3.53 (m, 1H), 3.54-3.37 (m, 12H), 3.31 (d, J=6.3 Hz, 2H), 2.93 (t, J=5.9 Hz, 2H), 2.56 (t, J=7.3 Hz, 2H), 1.99-1.80 (m, 1H), 1.70-1.04 (m, 11H).

### Example 86: (2-((2R,3S,4S,5S,6R)-3,4,5-trihydroxy-6-(3-methyl-4-(3-(4-(1-(15-oxo-15-(perfluorophenoxy)-3,6,9,12-tetraoxapentadecyl)-1H-1,2,3-triazol yl)butyl)ureido)phenoxy)tetrahydro-2H-pyran-2-yl)ethyl)phosphonic acid (Cpd. No. I-86)

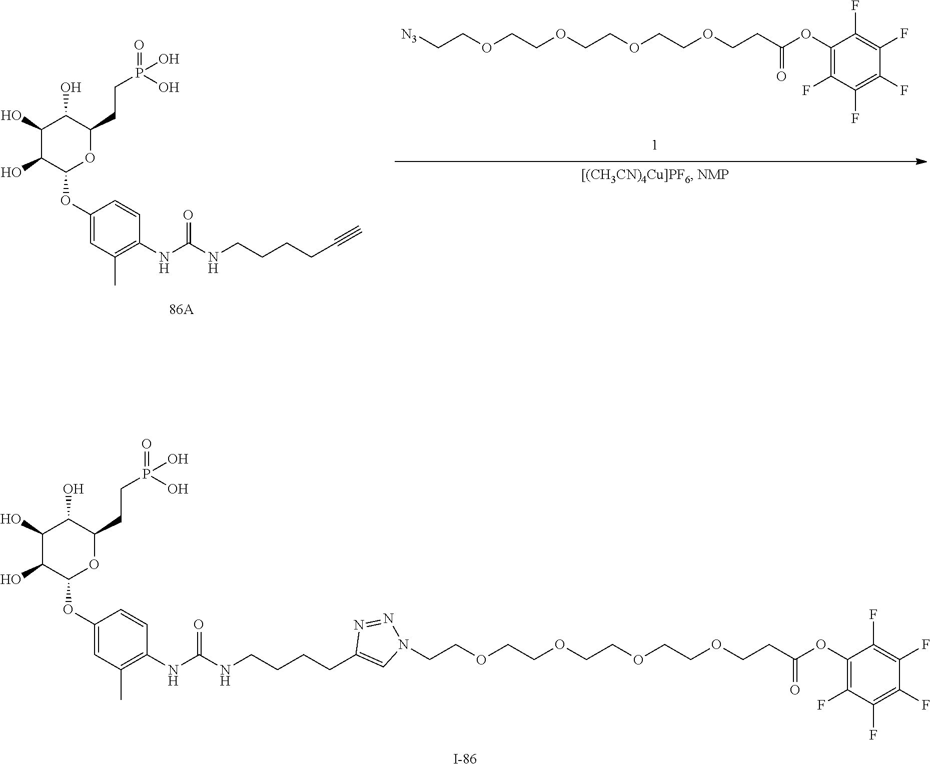

To (2-((2R,3S,4S,5S,6R)-6-(4-(3-(hex-5-yn-1-yl)ureido)-3-methylphenoxy)-3,4,5-trihydroxytetrahydro-2H-pyran-2-yl)ethyl)phosphonic acid (1.00 eq, 29.9 mg, 0.0614 mmol) in a 1 dram vial with a stirbar was added a solution of perfluorophenyl 1-azido-3,6,9,12-tetraoxapentadecan-15-oate (1, 1.20 eq, 33.7 mg, 0.0737 mmol) in NMP (0.5 mL) followed by tetrakis(acetonitrile)copper(I) hexafluorophosphate (2.50 eq, 57.2 mg, 0.154 mmol). The resulting clear yellow solution was capped and stirred at room temperature for 20 minutes (slowly turned green colored). The reaction was diluted with a mixture of NMP, ethanol, and acetic acid, filtered, and purified via preparatory HPLC (15-45% acetonitrile in water with 0.1% TFA). Fractions containing the desired product were combined and lyophilized to dryness to afford (2-((2R,3S,4S,5S,6R)-3,4,5-trihydroxy-6-(3-methyl-4-(3-(4-(1-(15-oxo-15-(perfluorophenoxy)-3,6,9,12-tetraoxapentadecyl)-1H-1,2,3-triazol-4-yl)butyl)ureido)phenoxy)tetrahydro-2H-pyran-2-yl)ethyl)phosphonic acid (Cpd. No. I-86) as a white solid. Yield: 37.4 mg, 65%; LCMS m/z 944.5 [M+1]⁺; ¹H NMR (300 MHz, DMSO-d₆ with D₂O) δ 7.77 (s, 1H), 7.43 (d, J=8.8 Hz, 1H), 6.81 (s, 1H), 6.75 (d, J=8.8 Hz, 1H), 5.23 (s, 1H), 4.42 (t, J=5.5 Hz, 2H), 3.97-3.68 (m, 5H), 3.64-3.56 (m, 1H), 3.54-3.38 (m, 12H), 3.35-3.27 (m, 2H), 3.04 (t, J=6.6 Hz, 2H), 2.98-2.89 (m, 2H), 2.59 (t, J=7.3 Hz, 2H), 2.09 (s, 3H), 1.98-1.81 (m, 1H), 1.69-1.34 (m, 6H), 1.31-1.10 (m, 1H).

### Example 87: Synthesis of (2-((2R,3S,4S,5S,6R)-3,4,5-trihydroxy-6-(2-methyl-4-(3-(4-(1-(15-oxo-15-(perfluorophenoxy)-3,6,9,12-tetraoxapentadecyl)-1H-1,2,3-triazol-4-yl)butyl)ureido)phenoxy)tetrahydro-2H-pyran-2-Methyl)phosphonic acid (Cpd. No. I-87)

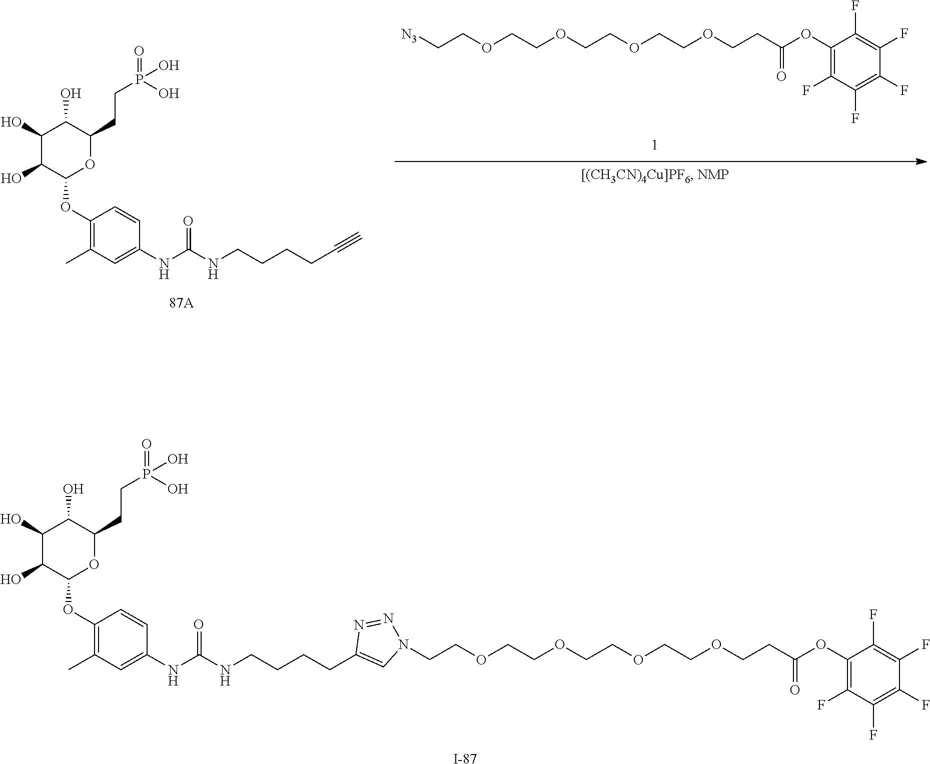

To (2-((2R,3S,4S,5S,6R)-6-(4-(3-(hex-5-yn-1-yl)ureido)-2-methylphenoxy)-3,4,5-trihydroxytetrahydro-2H-pyran-2-yl)ethyl)phosphonic acid (87A, 1.00 eq, 29.9 mg, 0.0615 mmol) in a 1 dram vial with a stirbar was added a solution of perfluorophenyl 1-azido-3,6,9,12-tetraoxapentadecan-15-oate (1, 1.20 eq, 33.8 mg, 0.0739 mmol) in NMP (0.5 mL) followed by tetrakis(acetonitrile)copper(I) hexafluorophosphate (2.50 eq, 57.4 mg, 0.154 mmol). The resulting clear yellow solution was capped and stirred at room temperature for 20 minutes (slowly turned green colored). The reaction was diluted with a mixture of NMP, ethanol, and acetic acid, filtered, and purified via preparatory HPLC (15-45% acetonitrile in water with 0.1% TFA). Fractions containing the desired product were combined and lyophilized to dryness to afford (2-((2R,3S,4S,5S,6R)-3,4,5-trihydroxy-6-(2-methyl-4-(3-(4-(1-(15-oxo-15-(perfluorophenoxy)-3,6,9,12-tetraoxapentadecyl)-1H-1,2,3-triazol-4-yl)butyl)ureido)phenoxy)tetrahydro-2H-pyran-2-yl)ethyl)phosphonic acid (Cpd. No. I-87) as a white solid. Yield: 39.8 mg, 69%; LCMS m/z 944.5 [M+1]+; ¹H NMR (300 MHz, DMSO-d₆ with D₂O) δ 7.77 (s, 1H), 7.12 (s, 1H), 7.06 (d, J=8.7 Hz, 1H), 6.88 (d, J=8.8 Hz, 1H), 5.22 (s, 1H), 4.41 (t, J=5.1 Hz, 2H), 3.97-3.68 (m, 5H), 3.67-3.59 (m, 1H), 3.55-3.39 (m, 12H), 3.37-3.22 (m, 2H), 3.02 (t, J=7.3 Hz, 2H), 2.94 (t, J=5.9 Hz, 2H), 2.59 (t, J=7.5 Hz, 2H), 2.08 (s, 3H), 1.98-1.82 (m, 1H), 1.71-1.33 (m, 6H), 1.30-1.09 (m, 1H).

### Example 88: (2-((2R,3S,4S,5S,6R)-3,4,5-trihydroxy-6-((3′-(4-(1-(15-oxo (perfluorophenoxy)-3,6,9,12-tetraoxapentadecyl)-1H-1,2,3-triazol-4-yl)butyl)-[1,1′-biphenyl]-4-yl)oxy)tetrahydro-2H-pyran-2-yl)ethyl)phosphonic acid (Cpd. No. I-88)

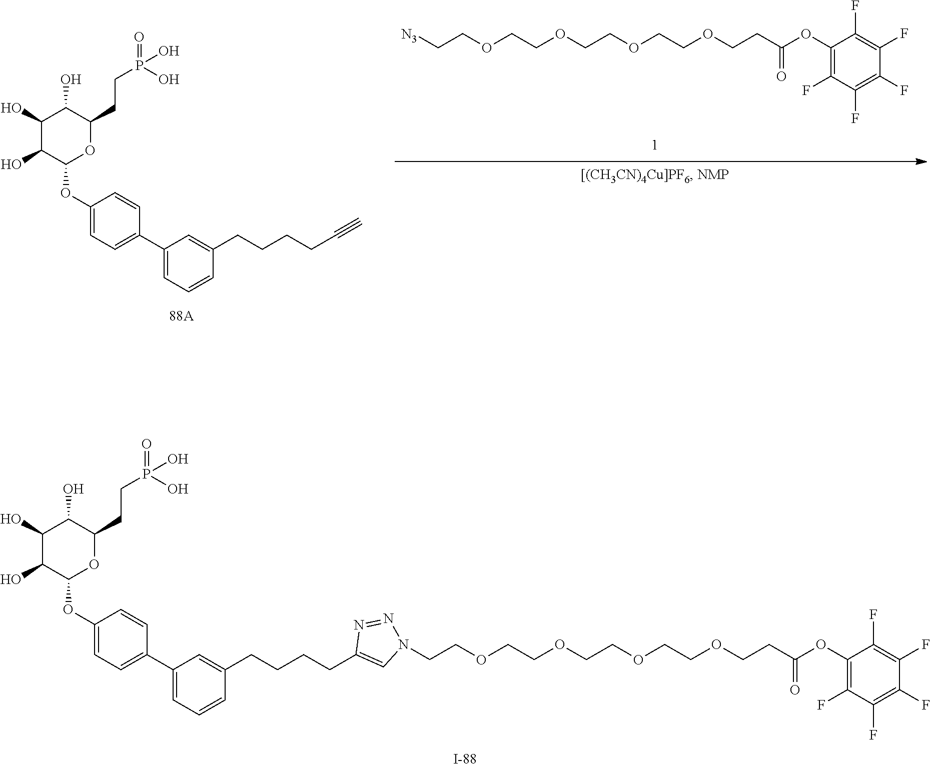

To (2-((2R,3S,4S,5S,6R)-6-((3′-(hex-5-yn-1-yl)-[1,1′-biphenyl]-4-yl)oxy)-3,4,5-trihydroxytetrahydro-2H-pyran-2-yl)ethyl)phosphonic acid (88A, 1.00 eq, 31.0 mg, 0.0632 mmol) in a 1 dram vial with a stirbar was added a solution of perfluorophenyl 1-azido-3,6,9,12-tetraoxapentadecan-15-oate (1, 1.20 eq, 34.7 mg, 0.0758 mmol) in NMP (0.5 mL) followed by tetrakis(acetonitrile)copper(I) hexafluorophosphate (2.50 eq, 58.9 mg, 0.158 mmol). The resulting clear yellow solution was capped and stirred at room temperature for 20 minutes (slowly turned green colored). The reaction was diluted with a mixture of NMP, ethanol, and acetic acid, filtered, and purified via preparatory HPLC (20-80% acetonitrile in water with 0.1% TFA). Fractions containing the desired product were combined and lyophilized to dryness to afford (Cpd. No. I-88) as a white solid. Yield: 40.5 mg, 68%; LCMS 948.5 m/z [M+1]⁺; ¹H NMR (300 MHz, DMSO-d₆ with D₂O) δ 7.74 (s, 1H), 7.55 (d, J=8.3 Hz, 2H), 7.36 (s, 2H), 7.30 (t, J=7.6 Hz, 1H), 7.08 (d, J=8.0 Hz, 3H), 5.39 (s, 1H), 4.40 (s, 2H), 3.79-3.58 (m, 5H), 3.53-3.23 (m, 15H), 2.92 (t, J=5.8 Hz, 2H), 2.68-2.56 (m, 4H), 2.01-1.80 (m, 1H), 1.68-1.43 (m, 6H), 1.28-1.06 (m, 1H).

### Example 89: (2-((2R,3S,4S,5S,6R)-6-(4-(3-(4-(1-(30-(2,5-dioxo-2,5-dihydro-1H-pyrrol-1-yl)-27-oxo-3,6,9,12,15,18,21,24-octaoxa-28-azatriacontyl)-1H-1,2,3-triazol yl)butyl)ureido)phenoxy)-3,4,5-trihydroxytetrahydro-2H-pyran-2-yl)ethyl)phosphonic acid (Cpd. No. I-89)

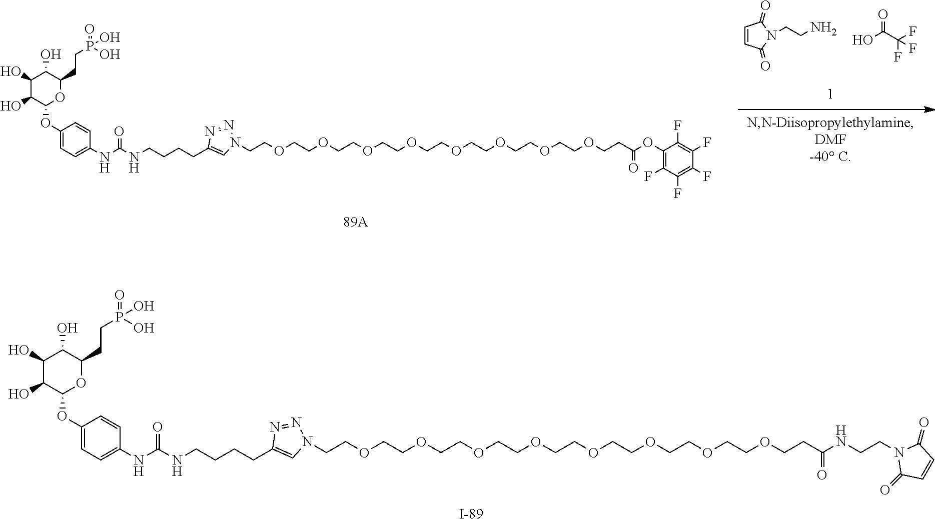

A solution of 1-(2-aminoethyl)-1H-pyrrole-2,5-dione TFA salt (1, 1.10 eq, 11.2 mg, 0.0443 mmol) and N,N-diisopropylethylamine (3.00 eq, 0.021 mL, 0.121 mmol) in DMF (0.3 mL) was added to a stirred solution of (2-((2R,3S,4S,5S,6R)-3,4,5-trihydroxy-6-(4-(3-(4-(1-(27-oxo-27-(perfluorophenoxy)-3,6,9,12,15,18,21,24-octaoxaheptacosyl)-1H-1,2,3-triazol-4-yl)butyl)ureido)phenoxy)tetrahydro-2H-pyran-2-yl)ethyl)phosphonic acid (89A, 1.00 eq, 44.5 mg, 0.0402 mmol)) in DMF (0.3 mL) at −40° C. The cold bath was allowed to slowly warm. The resulting solution was stirred for 40 minutes. The final temperature of the cold bath was −5° C. The reaction was diluted with acetic acid (0.4 mL), filtered, and purified via preparatory HPLC (10-30% acetonitrile in water with 0.1% TFA). Fractions containing the desired product were combined and lyophilized to dryness to afford (2-((2R,3S,4S,5S,6R)-6-(4-(3-(4-(1-(30-(2,5-dioxo-2,5-dihydro-1H-pyrrol-1-yl)-27-oxo-3,6,9,12,15,18,21,24-octaoxa-28-azatriacontyl)-1H-1,2,3-triazol-4-yl)butyl)ureido)phenoxy)-3,4,5-trihydroxytetrahydro-2H-pyran-2-yl)ethyl)phosphonic acid (Cpd. No. I-89) as a pale yellow solid. Yield: 24.2 mg, 57%; LCMS m/z 1062.6 [M+1]⁺; ¹H NMR (300 MHz, DMSO-d₆ with D₂O) δ 7.78 (s, 1H), 7.24 (d, J=8.5 Hz, 2H), 6.99-6.79 (m, 4H), 5.24 (s, 1H), 4.42 (bs, 2H), 3.79-3.70 (m, 4H), 3.64-3.55 (m, 1H), 3.56-3.36 (m, 31H), 3.35-3.28 (m, 2H), 3.23-3.12 (m, 2H), 3.11-2.99 (m, 2H), 2.67-2.55 (m, 2H), 2.24-2.12 (m, 2H), 2.02-1.77 (m, 1H), 1.71-1.34 (m, 6H), 1.32-1.04 (m, 1H).

### Example 90: (2-((2R,3S,4S,5S,6R)-6-(4-(4-(20-((2,5-dioxopyrrolidin-1-yl)oxy)-20-oxo-2,5,8,11,14,17-hexaoxaicosyl)-1H-1,2,3-triazol-1-yl)phenoxy)-3,4,5-trihydroxytetrahydro-2H-pyran-2-yl)ethyl)phosphonic acid (Cpd. No. I-90)

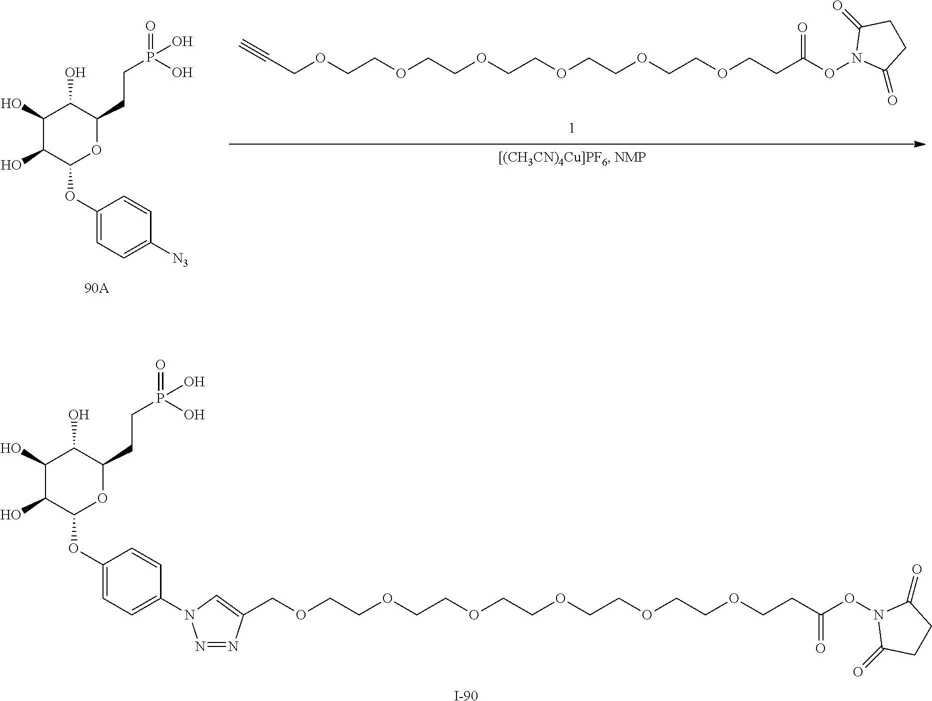

To (2-((2R,3S,4S,5S,6R)-6-(4-azidophenoxy)-3,4,5-trihydroxytetrahydro-2H-pyran-2-yl)ethyl)phosphonic acid (90A, 1.00 eq, 25.3 mg, 0.0675 mmol) in a 1 dram vial with a stirbar was added a solution of 2,5-dioxopyrrolidin-1-yl 4,7,10,13,16,19-hexaoxadocos-21-ynoate (1, 1.20 eq, 36.1 mg, 0.0810 mmol) in NMP (0.5 mL) followed by tetrakis(acetonitrile)copper(I) hexafluorophosphate (2.50 eq, 62.9 mg, 0.169 mmol). The resulting clear burgundy solution was capped and stirred at room temperature for 20 minutes (slowly turned green colored). The reaction showed 90% conversion to product. More tetrakis(acetonitrile)copper(I) hexafluorophosphate (2.50 eq, 62.9 mg, 0.169 mmol) was added, and the solution was stirred for an additional 20 minutes. The reaction was diluted with a mixture of NMP, ethanol, and acetic acid, filtered, and purified via preparatory HPLC (15-40% acetonitrile in water with 0.1% TFA). Fractions containing the desired product were combined and lyophilized to dryness to afford (2-((2R,3S,4S,5S,6R)-6-(4-(4-(20-((2,5-dioxopyrrolidin-1-yl)oxy)-20-oxo-2,5,8,11,14,17-hexaoxaicosyl)-1H-1,2,3-triazol-1-yl)phenoxy)-3,4,5-trihydroxytetrahydro-2H-pyran-2-yl)ethyl)phosphonic acid (Cpd. No. I-90) as a white solid. Yield: 32.3 mg, 58%; LCMS m/z 821.5 [M+1]+; ¹H NMR (300 MHz, DMSO-d₆ with D₂O) δ 8.58 (s, 1H), 7.83-7.71 (m, 2H), 7.29-7.17 (m, 2H), 5.47 (s, 1H), 4.58 (s, 2H), 3.89-3.80 (m, 2H), 3.72-3.62 (m, 3H), 3.62-3.23 (m, 21H), 2.85 (t, J=5.9 Hz, 2H), 2.77 (s, 4H), 2.00-1.82 (m, 1H), 1.70-1.40 (m, 2H), 1.27-1.03 (m, 1H).

### Example 91: (2-((2R,3S,4S,5S,6R)-3,4,5-trihydroxy-6-(4-(1-(21-oxo (perfluorophenoxy)-3,6,9,12,15,18-hexaoxahenicosyl)-1H-1,2,3-triazol-4-yl)phenoxy)tetrahydro-2H-pyran-2-yl)ethyl)phosphonic acid (Cpd. No. I-91)

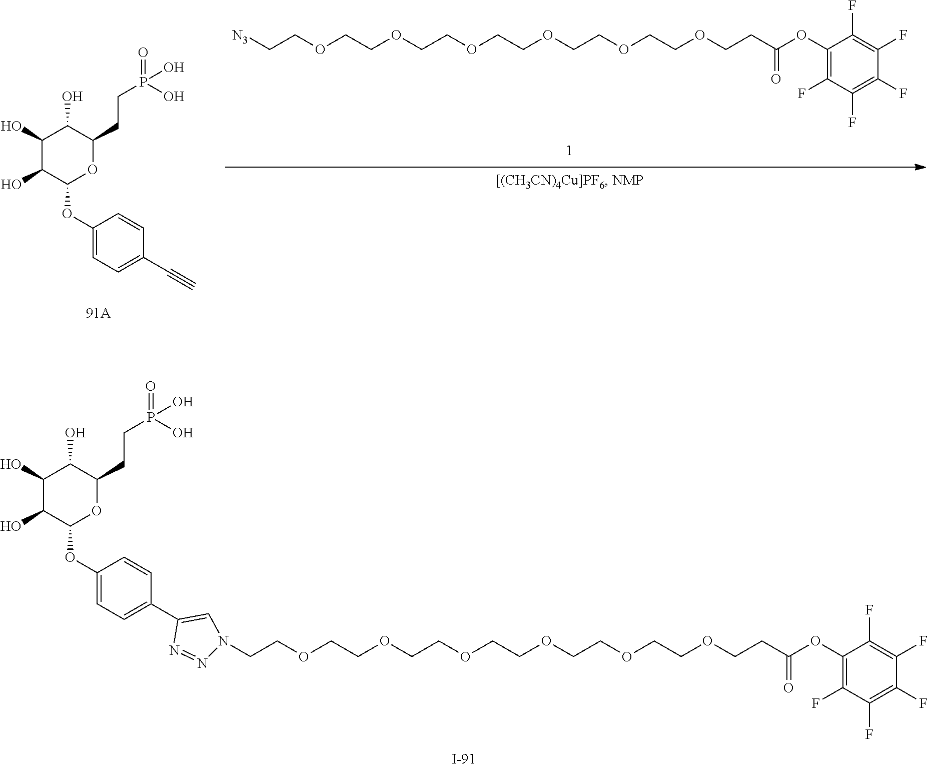

To (2-((2R,3S,4S,5S,6R)-6-(4-ethynylphenoxy)-3,4,5-trihydroxytetrahydro-2H-pyran-2-yl)ethyl)phosphonic acid (91A, 1.00 eq, 25.5 mg, 0.0711 mmol) in a 1 dram vial with a stirbar was added a solution of perfluorophenyl 1-azido-3,6,9,12,15,18-hexaoxahenicosan-21-oate (1, 1.20 eq, 46.5 mg, 0.0853 mmol) in NMP (0.5 mL) followed by tetrakis(acetonitrile)copper(I) hexafluorophosphate (2.50 eq, 66.2 mg, 0.178 mmol). The resulting clear yellow solution was capped and stirred at room temperature for 20 minutes (slowly turned green colored). The reaction was diluted with a mixture of NMP, ethanol, and acetic acid, filtered, and purified via preparatory HPLC (20-60% acetonitrile in water with 0.1% TFA). Fractions containing the desired product were combined and lyophilized to dryness to afford (2-((2R,3S,4S,5S,6R)-3,4,5-trihydroxy-6-(4-(1-(21-oxo-21-(perfluorophenoxy)-3,6,9,12,15,18-hexaoxahenicosyl)-1H-1,2,3-triazol-4-yl)phenoxy)tetrahydro-2H-pyran-2-yl)ethyl)phosphonic acid (Cpd. No. I-91) as a white solid. Yield: 43.7 mg, 68%; LCMS m/z 904.5 [M+1]⁺; ¹H NMR (300 MHz, DMSO-d₆ with D₂O) δ 8.35 (s, 1H), 7.73 (d, J=8.0 Hz, 2H), 7.09 (d, J=8.3 Hz, 2H), 5.40 (s, 1H), 4.51 (t, J=5.0 Hz, 2H), 3.85-3.79 (m, 3H), 3.72 (t, J=5.8 Hz, 2H), 3.64 (dd, J=8.8, 3.4 Hz, 1H), 3.53-3.23 (m, 22H), 2.94 (t, J=5.8 Hz, 2H), 2.01-1.78 (m, 1H), 1.71-1.38 (m, 2H), 1.28-1.00 (m, 1H).

### Example 92: (2-((2R,3S,4S,5S,6R)-3,4,5-trihydroxy-6-(3-hydroxy-4-(3-(4-(1-(15-oxo-15-(perfluorophenoxy)-3,6,9,12-tetraoxapentadecyl)-1H-1,2,3-triazol yl)butyl)ureido)phenoxy)tetrahydro-2H-pyran-2-yl)ethyl)phosphonic acid (Cpd. No. I-92)

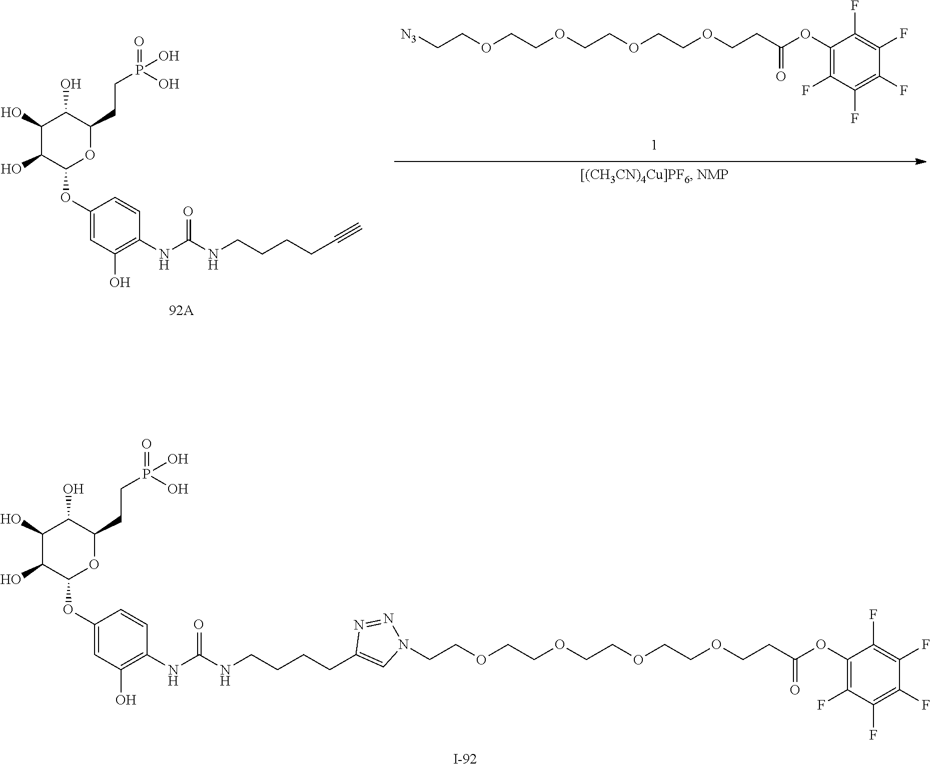

To (2-((2R,3S,4S,5S,6R)-6-(4-(3-(hex-5-yn-1-yl)ureido)-3-hydroxyphenoxy)-3,4,5-trihydroxytetrahydro-2H-pyran-2-yl)ethyl)phosphonic acid (92A, 1.00 eq, 30.4 mg, 0.0622 mmol) in a 1 dram vial with a stirbar was added a solution of perfluorophenyl 1-azido-3,6,9,12-tetraoxapentadecan-15-oate (1, 1.20 eq, 34.2 mg, 0.0747 mmol) in NMP (0.5 mL) followed by tetrakis(acetonitrile)copper(I) hexafluorophosphate (2.50 eq, 58.0 mg, 0.156 mmol). The resulting clear amber solution was capped and stirred at room temperature for 20 minutes (slowly turned green colored). The reaction was diluted with a mixture of NMP, ethanol, and acetic acid, filtered, and purified via preparatory HPLC (15-60% acetonitrile in water with 0.1% TFA). Fractions containing the desired product were combined and lyophilized to dryness to afford (2-((2R,3S,4S,5S,6R)-3,4,5-trihydroxy-6-(3-hydroxy-4-(3-(4-(1-(15-oxo-15-(perfluorophenoxy)-3,6,9,12-tetraoxapentadecyl)-1H-1,2,3-triazol yl)butyl)ureido)phenoxy)tetrahydro-2H-pyran-2-yl)ethyl)phosphonic acid (Cpd. No. I-92) as a white solid. Yield: 42.0 mg, 71%; LCMS m/z 946.5 [M+1]⁺; ¹H NMR (300 MHz, DMSO-d₆ with D₂O) δ 7.77 (s, 1H), 7.57 (d, J=8.8 Hz, 1H), 6.53-6.45 (m, 1H), 6.43-6.32 (m, 1H), 5.19 (s, 1H), 4.41 (t, J=5.0 Hz, 2H), 3.80-3.68 (m, 5H), 3.61-3.54 (m, 1H), 3.53-3.38 (m, 12H), 3.33-3.27 (m, 2H), 3.04 (t, J=6.9 Hz, 2H), 2.93 (t, J=5.8 Hz, 2H), 2.59 (t, J=7.4 Hz, 2H), 2.00-1.83 (m, 1H), 1.74-1.34 (m, 6H), 1.34-1.12 (m, 1H).

### Example 93: (2-((2R,3S,4S,5S,6R)-3,4,5-trihydroxy-6-(2-hydroxy-4-(3-(4-(1-(15-oxo-15-(perfluorophenoxy)-3,6,9,12-tetraoxapentadecyl)-1H-1,2,3-triazol-4-yl)butyl)ureido)phenoxy)tetrahydro-2H-pyran-2-yl)ethyl)phosphonic acid (Cpd. No. I-93)

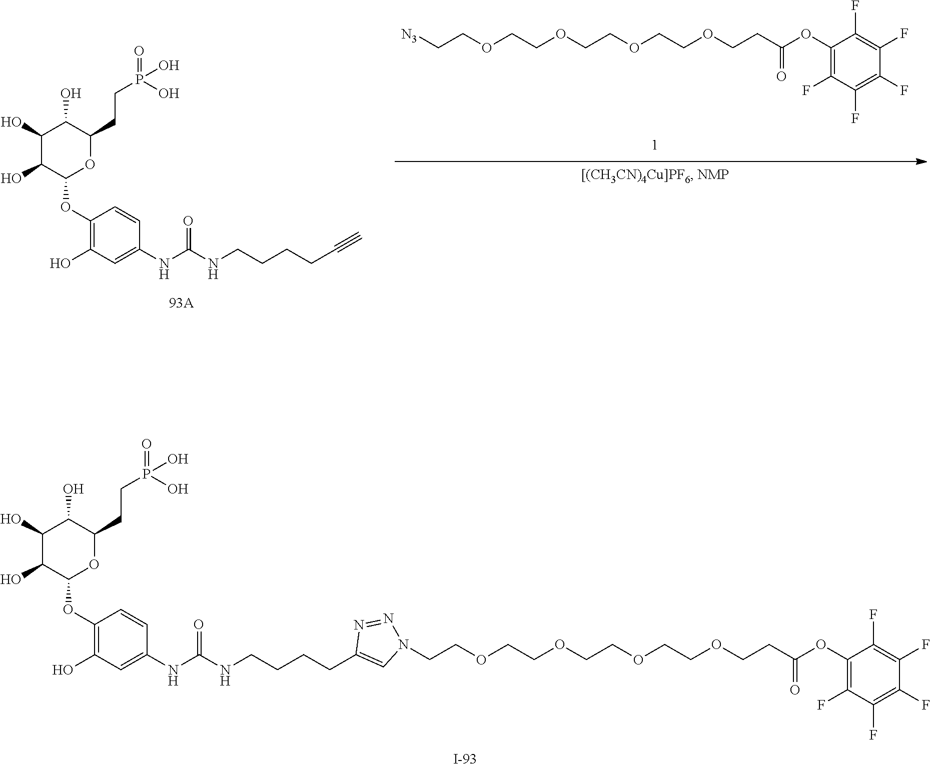

To (2-((2R,3S,4S,5S,6R)-6-(4-(3-(hex-5-yn-1-yl)ureido)-2-hydroxyphenoxy)-3,4,5-trihydroxytetrahydro-2H-pyran-2-yl)ethyl)phosphonic acid (93A, 1.00 eq, 30.3 mg, 0.0620 mmol) in a 1 dram vial with a stirbar was added a solution of perfluorophenyl 1-azido-3,6,9,12-tetraoxapentadecan-15-oate (1, 1.20 eq, 34.0 mg, 0.0744 mmol) in NMP (0.5 mL) followed by tetrakis(acetonitrile)copper(I) hexafluorophosphate (2.50 eq, 57.8 mg, 0.155 mmol). The resulting clear orange solution was capped and stirred at room temperature for 20 minutes (turned green). The reaction mixture was diluted with a mixture of NMP, ethanol, and acetic acid, filtered, and purified via preparatory HPLC (15-60% acetonitrile in water with 0.1% TFA). Fractions containing the desired product were combined and lyophilized to dryness to afford (2-((2R,3S,4S,5S,6R)-3,4,5-trihydroxy-6-(2-hydroxy-4-(3-(4-(1-(15-oxo (perfluorophenoxy)-3,6,9,12-tetraoxapentadecyl)-1H-1,2,3-triazol yl)butyl)ureido)phenoxy)tetrahydro-2H-pyran-2-yl)ethyl)phosphonic acid (Cpd. No. I-93) as a white solid. Yield: 43.6 mg, 74%; LCMS m/z 946.5 [M+1]⁺; ¹H NMR (300 MHz, DMSO-d₆ with D₂O) δ 7.77 (s, 1H), 7.02-6.96 (m, 1H), 6.83 (d, J=8.7 Hz, 1H), 6.64-6.54 (m, 1H), 5.11 (s, 1H), 4.41 (t, J=5.2 Hz, 2H), 3.88-3.85 (m, 1H), 3.79-3.60 (m, 5H), 3.57-3.36 (m, 13H), 3.30 (t, J=9.4 Hz, 1H), 3.03 (t, J=6.7 Hz, 2H), 3.03-2.88 (m, 2H), 2.59 (t, J=7.4 Hz, 2H), 2.02-1.81 (m, 1H), 1.69-1.14 (m, 7H).

### Example 94: (2-((2R,3S,4S,5S,6R)-3,4,5-trihydroxy-6-((6-(4-(1-(15-oxo-15-(perfluorophenoxy)-3,6,9,12-tetraoxapentadecyl)-1H-1,2,3-triazol-4-yl)butanamido)naphthalen-2-yl)oxy)tetrahydro-2H-pyran-2-yl)ethyl)phosphonic acid (Cpd. No. I-94)

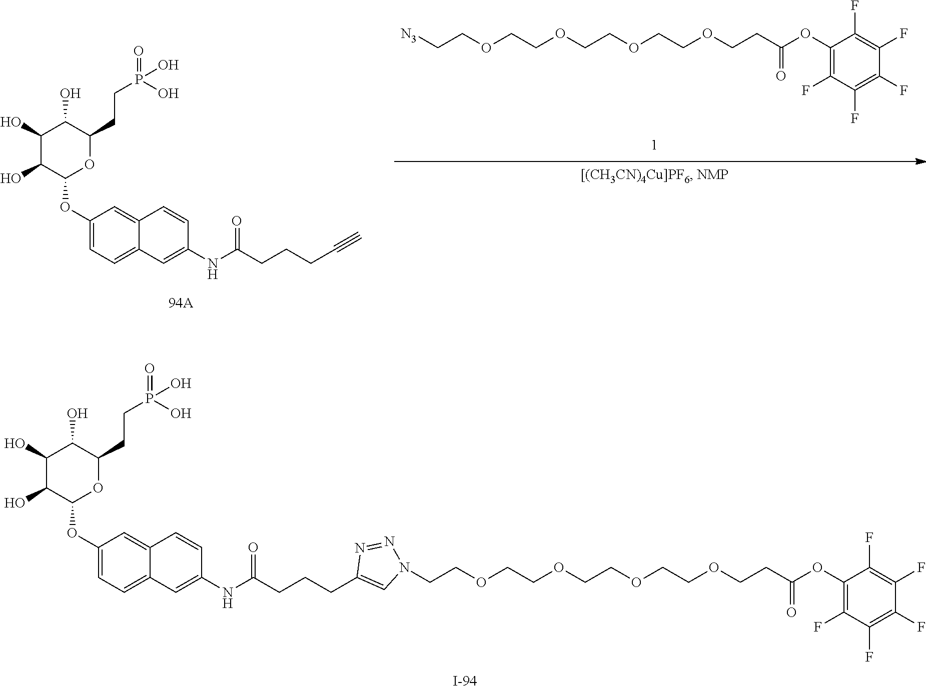

To (2-((2R,3S,4S,5S,6R)-6-((6-(hex-5-ynamido)naphthalen-2-yl)oxy)-3,4,5-trihydroxytetrahydro-2H-pyran-2-yl)ethyl)phosphonic acid (1.00 eq, 25.1 mg, 0.0509 mmol) in a 1 dram vial with a stirbar was added a solution of perfluorophenyl 1-azido-3,6,9,12-tetraoxapentadecan-15-oate (1, 1.20 eq, 27.9 mg, 0.0610 mmol) in NMP (0.4 mL) followed by tetrakis(acetonitrile)copper(I) hexafluorophosphate (2.50 eq, 47.4 mg, 0.127 mmol). The resulting colourless solution was capped and stirred at room temperature for 20 minutes (slowly turned green colored). The reaction was diluted with a mixture of NMP, ethanol, and acetic acid, filtered, and purified via preparatory HPLC (20-70% acetonitrile in water with 0.1% TFA). Fractions containing the desired product were combined and lyophilized to dryness to afford (2-((2R,3S,4S,5S,6R)-3,4,5-trihydroxy-6-((6-(4-(1-(15-oxo (perfluorophenoxy)-3,6,9,12-tetraoxapentadecyl)-1H-1,2,3-triazol yl)butanamido)naphthalen-2-yl)oxy)tetrahydro-2H-pyran-2-yl)ethyl)phosphonic acid (Cpd. No. I-94) as a white solid. Yield: 36.7 mg, 76%; LCMS m/z 951.5 [M+1]⁺; ¹H NMR (300 MHz, DMSO-d₆ with D₂O) δ 8.15 (s, 1H), 7.81 (s, 1H), 7.71 (d, J=8.9 Hz, 2H), 7.49 (d, J=8.9 Hz, 1H), 7.39 (d, J=2.4 Hz, 1H), 7.19 (dd, J=8.8, 2.4 Hz, 1H), 5.47 (s, 1H), 4.43 (t, J=5.0 Hz, 2H), 3.79-3.63 (m, 5H), 3.52-3.27 (m, 15H), 2.91 (t, J=5.8 Hz, 2H), 2.65 (t, J=7.6 Hz, 2H), 2.38 (t, J=7.4 Hz, 2H), 1.97-1.82 (m, 3H), 1.70-1.39 (m, 2H), 1.24-1.01 (m, 1H).

### Example 95: (2-((2R,3S,4S,5S,6R)-3,4,5-trihydroxy-6-((4-(3-(4-(1-(15-oxo-15-(perfluorophenoxy)-3,6,9,12-tetraoxapentadecyl)-1H-1,2,3-triazol-4-yl)butyl)ureido)phenyl)thio)tetrahydro-2H-pyran-2-yl)ethyl)phosphonic acid (Cpd. No. I-95)

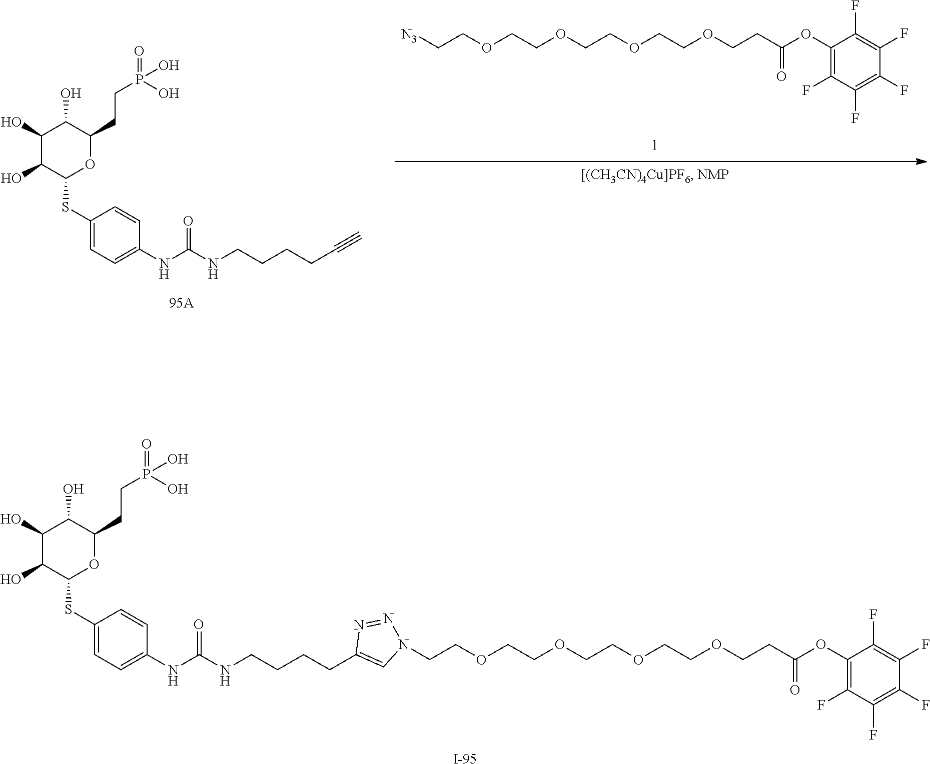

To (2-((2R,3S,4S,5S,6R)-6-((4-(3-(hex-5-yn-1-yl)ureido)phenyl)thio)-3,4,5-trihydroxytetrahydro-2H-pyran-2-yl)ethyl)phosphonic acid (95A, 1.00 eq, 25.5 mg, 0.0522 mmol) in a 1 dram vial with a stirbar was added a solution of perfluorophenyl 1-azido-3,6,9,12-tetraoxapentadecan-15-oate (1, 1.20 eq, 28.6 mg, 0.0626 mmol) in NMP (0.4 mL) followed by tetrakis(acetonitrile)copper(I) hexafluorophosphate (2.50 eq, 48.6 mg, 0.131 mmol). The resulting colourless solution was capped and stirred at room temperature for 20 minutes (slowly turned green colored). The reaction was diluted with a mixture of NMP, ethanol, and acetic acid, filtered, and purified via preparatory HPLC (20-70% acetonitrile in water with 0.1% TFA). Fractions containing the desired product were combined and lyophilized to dryness to afford (2-((2R,3S,4S,5S,6R)-3,4,5-trihydroxy-6-((4-(3-(4-(1-(15-oxo-15-(perfluorophenoxy)-3,6,9, 12-tetraoxapentadecyl)- 1H-1,2, 3-triazol yl)butyl)ureido)phenyl)thio)tetrahydro-2H-pyran-2-yl)ethyl)phosphonic acid (Cpd. No. I-95) as a white solid. Yield: 36.3 mg, 74%; LCMS m/z 946.5 [M+1]⁺; ¹H NMR (300 MHz, DMSO-d₆ with D₂O) δ 7.77 (s, 1H), 7.36-7.23 (m, 4H), 5.14 (s, 1H), 4.42 (t, J=4.9 Hz, 2H), 3.90-3.64 (m, 6H), 3.55-3.37 (m, 13H), 3.32 (t, J=9.3 Hz, 1H), 3.06 (t, J=6.7 Hz, 2H), 2.95 (t, J=5.5 Hz, 2H), 2.59 (t, J=7.4 Hz, 2H), 2.04-1.87 (m, 1H), 1.64-1.27 (m, 7H).

### Example 96: (2-((2R,3S,4S,5S,6R)-6-(4-(3-(4-(1-(27-(2,5-dioxo-2,5-dihydro-1H-pyrrol-1-yl)-12-(2-(2-(2-(2-(4-(4-(3-(3-hydroxy-4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol-1-yl)ethoxy)ethoxy)ethoxy)ethyl)-24-oxo-3,6,9,15,18,21-hexaoxa-12,25-diazaheptacosyl)-1H-1,2,3-triazol-4-yl)butyl)ureido)-2-hydroxyphenoxy)-3,4,5-trihydroxytetrahydro-2H-pyran-2-yl)ethyl)phosphonic acid (Cpd. No. I-96)

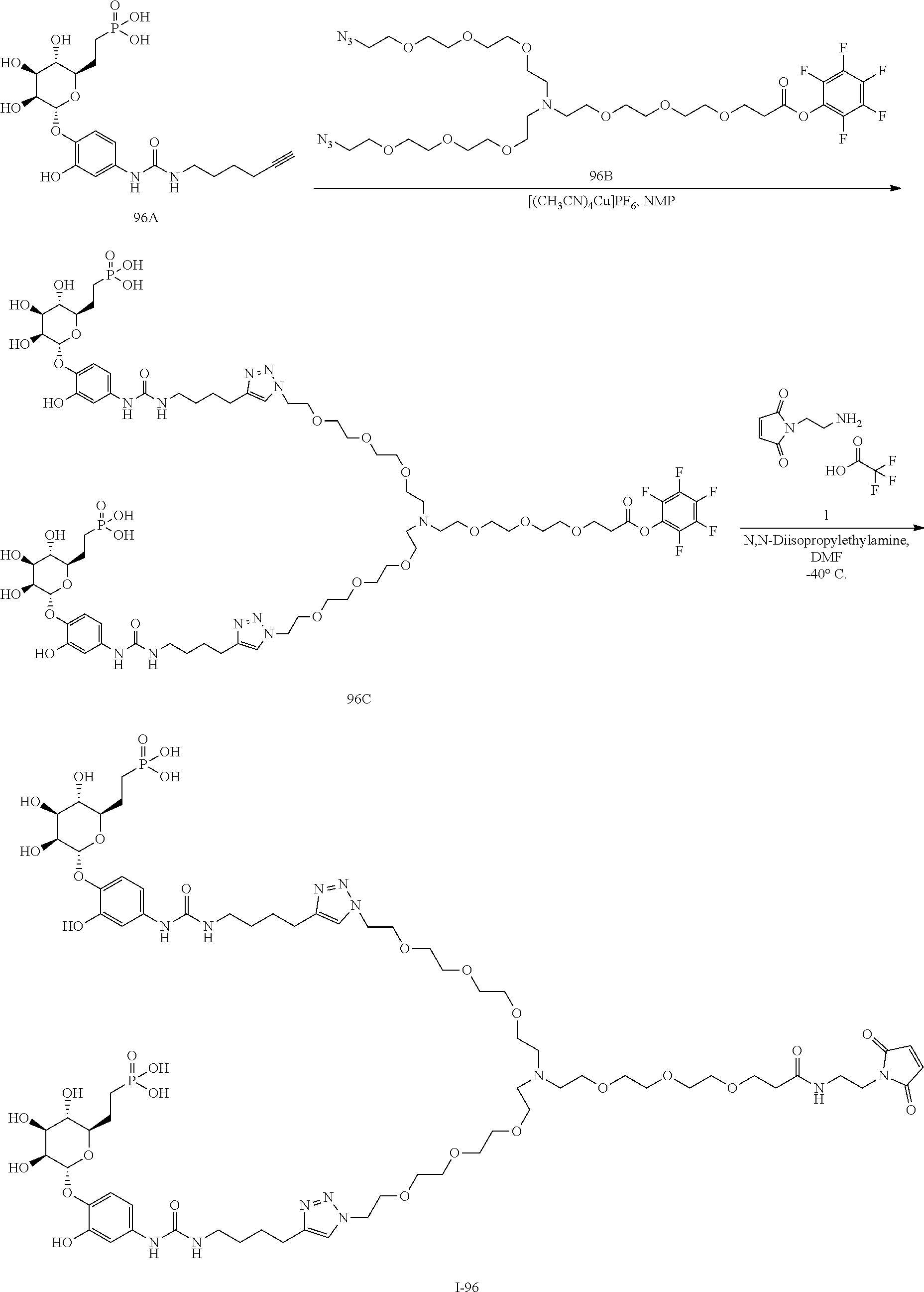

A solution of (2-((2R,3S,4S,5S,6R)-6-(4-(3-(hex-5-yn-1-yl)ureido)-2-hydroxyphenoxy)-3,4,5-trihydroxytetrahydro-2H-pyran-2-yl)ethyl)phosphonic acid (96A, 2.10 eq, 48.0 mg, 0.0982 mmol) in NMP (0.6 mL) was added to perfluorophenyl 1-azido-12-(2-(2-(2-(2-azidoethoxy)ethoxy)ethoxy)ethyl)-3,6,9,15,18,21-hexaoxa-12-azatetracosan-24-oate) (96B, 1.00 eq, 36.9 mg, 0.0467 mmol) in a 1 dram vial with a stirbar, followed by tetrakis(acetonitrile)copper(I) hexafluorophosphate (5.00 eq, 87.1 mg, 0.234 mmol). The resulting clear orange solution was capped and stirred at room temperature for 15 minutes (slowly turned more green-colored). The reaction mixture was diluted with acetic acid, filtered, and purified via preparatory HPLC (15-40% acetonitrile in water with 0.1% TFA). Fractions containing the desired product were combined and lyophilized to dryness to afford (96C) as a white solid. Yield: 37.2 mg, 45%; LCMS m/z 1765.9 [M−1]−; ¹H NMR (300 MHz, DMSO-d₆ with D₂O) δ 7.74 (s, 2H), 6.96 (s, 2H), 6.84 (d, J=8.8 Hz, 2H), 6.59 (d, J=8.6 Hz, 2H), 5.12 (s, 2H), 4.40 (bs, 4H), 3.88 (bs, 2H), 3.79-3.57 (m, 14H), 3.56-3.24 (m, 34H), 3.09-2.96 (m, 4H), 2.96-2.85 (m, 2H), 2.63-2.53 (m, 4H), 2.01-1.82 (m, 2H), 1.71-1.21 (m, 14H).

A solution of 1-(2-aminoethyl)-1H-pyrrole-2,5-dione TFA salt (1, 1.05 eq, 3.7 mg, 0.0147 mmol) and N,N-diisopropylethylamine (3.00 eq, 0.0073 mL, 0.0421 mmol) in DMF (0.1 mL) was added to a stirred solution of (2-((2R,3S,4S,5S,6R)-3,4,5-trihydroxy-6-(2-hydroxy-4-(3-(4-(1-(12-(2-(2-(2-(2-(4-(4-(3-(3-hydroxy-4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol yl)ethoxy)ethoxy)ethoxy)ethyl)-24-oxo-24-(perfluorophenoxy)-3,6,9,15,18,21-hexaoxa azatetracosyl)-1H-1,2,3-triazol-4-yl)butyl)ureido)phenoxy)tetrahydro-2H-pyran yl)ethyl)phosphonic acid (96C, 1.00 eq, 24.8 mg, 0.0140 mmol) in DMF (0.4 mL) at −40° C. under nitrogen. The resulting clear reaction solution was stirred vigorously under nitrogen while slowly warming for 25 minutes. A visual check showed that the solution had turned into a clear, viscous gel that prevented the stirbar from moving. The reaction mixture was removed from the cold bath at 39 minutes and −11.4° C. Shortly thereafter, the stirbar resumed its stirring and the timer was reset. After 10 minutes, the reaction showed 45% conversion to product. An additional solution of 1-(2-aminoethyl)-1H-pyrrole-2,5-dione TFA salt (0.65 eq, 2.31 mg, 0.009 mol) and N,N-diisopropylethylamine (1.85 eq, 0.004 mL, 0.026 mmol) in DMF (0.1 mL) was added to the stirred reaction mixture at −20° C. under nitrogen. The reaction mixture was removed from the cold bath and stirred vigorously under nitrogen while allowed to warm at room temperature. The reaction was stopped at 50 minutes. The reaction mixture was diluted with acetic acid, filtered, and purified via preparatory HPLC (5-30% acetonitrile in water with 0.1% TFA). Fractions containing the desired product were combined and lyophilized to dryness to afford (Cpd. No. I-96) as a white solid. Yield: 10.6 mg, 44%; LCMS m/z 1722.1 [M−1]−; ¹H NMR (300 MHz, DMSO-d₆ with D₂O) δ 7.75 (s, 2H), 6.98 (s, 2H), 6.88-6.79 (m, 4H), 6.59 (d, J=8.6 Hz, 2H), 5.11 (s, 2H), 4.45-4.35 (m, 4H), 3.87 (bs, 2H), 3.76-3.60 (m, 12H), 3.55-3.25 (m, 38H), 3.20-3.11 (m, 2H), 3.08-2.96 (m, 4H), 2.63-2.54 (m, 4H), 2.25-2.14 (m, 2H), 1.97-1.82 (m, 2H), 1.65-1.12 (m, 14H).

### Example 97: (2-((2R,3S,4S,5S,6R)-3,4,5-Trihydroxy-6-(4-(3-(4-(1-((S)-1,17,20,25-tetraoxo-1-(perfluorophenoxy)-18-(4-(5-(4-(4-(3-(4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol-1-yl)pentanamido)butyl)-4,7,10,13-tetraoxa-16,19,24-triazanonacosan-29-yl)-1H-1,2,3-triazol-4-yl)butyl)ureido)phenoxy)tetrahydro-2H-pyran-2-yl)ethyl)phosphonic acid (Cpd. No. I-97)

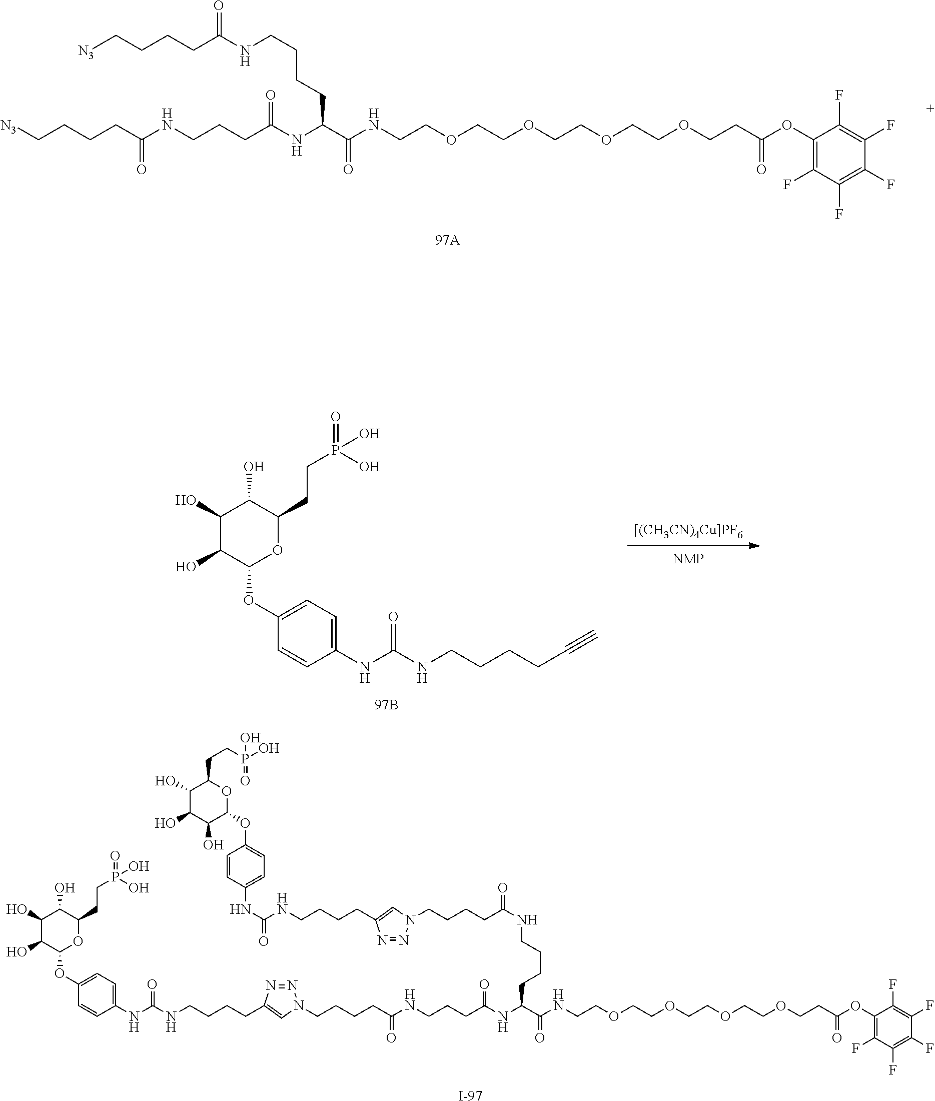

A solution of (2-((2R,3S,4S,5S,6R)-6-(4-(3-(hex-5-yn-1-yl)ureido)phenoxy)-3,4,5-trihydroxytetrahydro-2H-pyran-2-yl)ethyl)phosphonic acid (97B, 2.05 eq, 33.5 mg, 0.0709 mmol) in NMP (0.5 mL) was added to perfluorophenyl (S)-29-azido-18-(4-(5-azidopentanamido)butyl)-17,20,25-trioxo-4,7,10,13-tetraoxa-16,19,24-triazanonacosanoate (97A, 1.00 eq, 31.0 mg, 0.0346 mmol) in a 1 dram vial with a stirbar. The resulting solution was stirred for a few min before adding tetrakis(acetonitrile)copper(1) hexafluorophosphate (5.00 eq, 64.6 mg, 0.173 mmol). The resulting yellowish green solution was capped and stirred at room temperature for 20 min. The residue was diluted with AcOH, filtered, and purified via preparatory HPLC (15-60% acetonitrile in water with 0.1% TFA). Fractions containing the desired product were combined and lyophilized to dryness to afford (2-((2R,3S,4S,5S,6R)-3,4,5-trihydroxy-6-(4-(3-(4-(1-((S)-1,17,20,25-tetraoxo-1-(perfluorophenoxy)-18-(4-(5-(4-(4-(3-(4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol-1-yl)pentanamido)butyl)-4,7,10,13-tetraoxa-16,19,24-triazanonacosan-29-yl)-1H-1,2,3-triazol-4-yl)butyl)ureido)phenoxy)tetrahydro-2H-pyran-2-yl)ethyl)phosphonic acid (Cpd. No. I-97) as a white solid. Yield: 45 mg, 71%; LCMS m/z 1840.2 [M+1]+; ¹H NMR (300 MHz, DMSO-d₆ with D₂O) δ 7.84 (s, 2H), 7.30 (d, J=9.0 Hz, 4H), 6.95 (d, J=9.1 Hz, 4H), 5.30 (d, J=1.9 Hz, 2H), 4.31 (t, J=6.7 Hz, 4H), 4.18-4.13 (m, 1H), 3.88-3.64 (m, 4H), 3.61-3.32 (m, 18H), 3.30-2.93 (m, 14H), 2.68-2.61 (m, 4H), 2.20-2.06 (m, 5H), 2.03-1.90 (m, 1H), 1.84-1.17 (m, 32H).

### Example 98: (2-((2R,3S,4S,5S,6R)-6-(4-(3-(4-(1-((S)-1-(2,5-Dioxo-2,5-dihydro-1H-pyrrol-1-yl)-4,20,23,28-tetraoxo-21-(4-(5-(4-(4-(3-(4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol-1-yl)pentanamido)butyl)-7,10,13,16-tetraoxa-3,19,22,27-tetraazadotriacontan-32-yl)-1H-1,2,3-triazol-4-yl)butyl)ureido)phenoxy)-3,4,5-trihydroxytetrahydro-2H-pyran-2-yl)ethyl)phosphonic acid (Cpd. No. I-98)

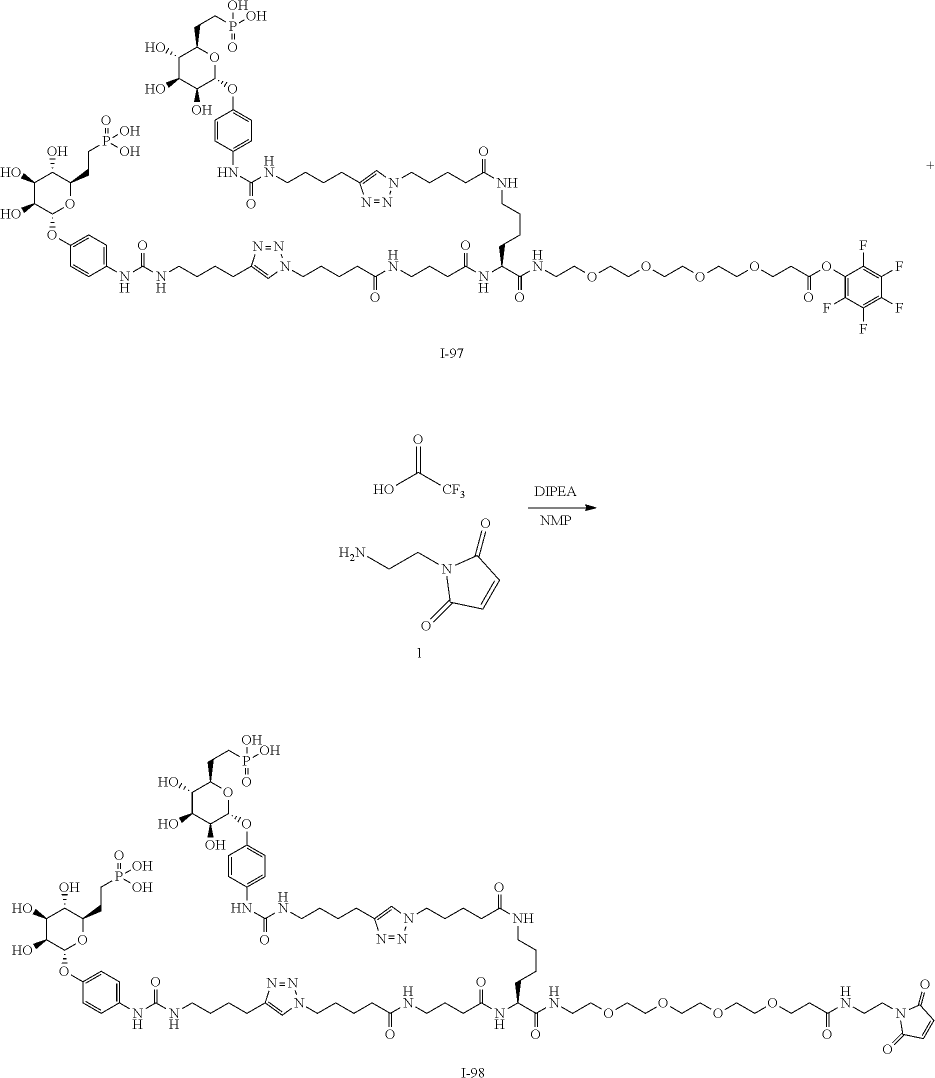

To (2-((2R,3S,4S,5S,6R)-3,4,5-trihydroxy-6-(4-(3-(4-(1-((S)-1,17,20,25-tetraoxo-1-(perfluorophenoxy)-18-(4-(5-(4-(4-(3-(4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol-1-yl)pentanamido)butyl)-4,7,10,13-tetraoxa-16,19,24-triazanonacosan-29-yl)-1H-1,2,3-triazol-4-yl)butyl)ureido)phenoxy)tetrahydro-2H-pyran-2-yl)ethyl)phosphonic acid (I-97, 1.00 eq, 35.0 mg, 0.0190 mmol) in a vial with a stir bar was added a solution of 1-(2-aminoethyl)-1H-pyrrole-2,5-dione TFA salt (1, 1.15 eq, 5.6 mg, 0.0219 mmol) and N,N-diisopropylethylamine (3.00 eq, 0.0099 mL, 0.0571 mmol) in NMP (0.5 mL) at −20° C. The resulting clear solution was capped and stirred and allowed to gradually warm over 1 h. The reaction was diluted with AcOH, filtered, and purified via preparatory HPLC (10-30% acetonitrile in water with 0.1% TFA). Fractions containing the desired product were combined and lyophilized to dryness to afford (2-((2R,3S,4S,5S,6R)-6-(4-(3-(4-(1-((S)-1-(2,5-dioxo-2,5-dihydro-1H-pyrrol-1-yl)-4,20,23,28-tetraoxo-21-(4-(5-(4-(4-(3-(4-(((2R,3S,4S,5S,6R)-3,4,5-trihydroxy-6-(2-phosphonoethyl)tetrahydro-2H-pyran-2-yl)oxy)phenyl)ureido)butyl)-1H-1,2,3-triazol yl)pentanamido)butyl)-7,10,13,16-tetraoxa-3,19,22,27-tetraazadotriacontan-32-yl)-1H-1,2,3-triazol-4-yl)butyl)ureido)phenoxy)-3,4,5-trihydroxytetrahydro-2H-pyran-2-yl)ethyl)phosphonic acid (Cpd. No. I-98) as a white solid. Yield: 19 mg, 55%; LCMS m/z 1796.0 [M+1]⁺; ¹H NMR (300 MHz, DMSO-d₆ with D₂O) δ 7.75 (s, 2H), 7.21 (d, J=8.9 Hz, 4H), 6.91-6.80 (m, 6H), 5.21 (d, J=1.9 Hz, 2H), 4.22 (t, J=6.9 Hz, 4H), 4.11-4.04 (m, 1H), 3.61-3.53 (m, 2H), 3.51-3.23 (m, 22H), 3.18-2.89 (m, 14H), 2.60-2.51 (m, 4H), 2.16 (t, J=6.5 Hz, 2H), 2.09-1.83 (m, 6H), 1.77-1.10 (m, 32H).

### Example 99: Compound I-99

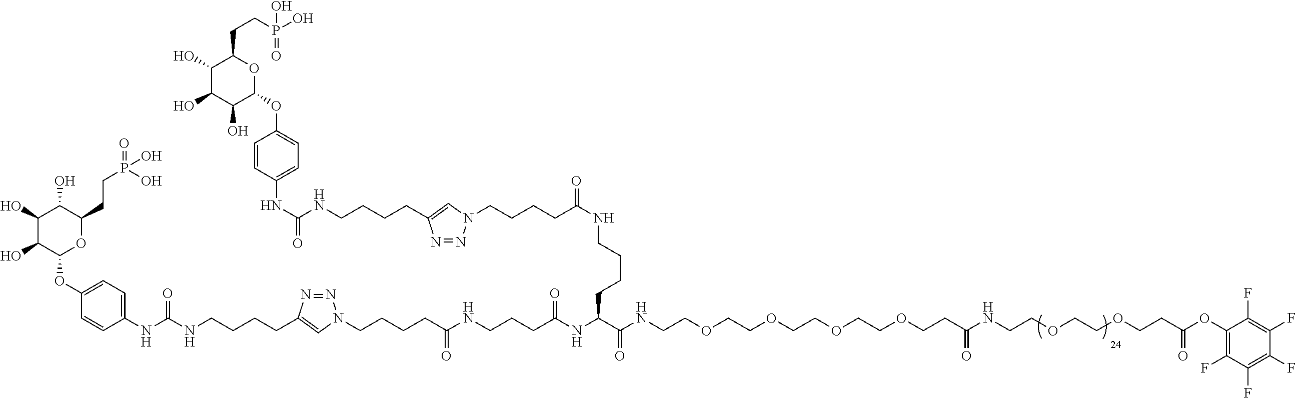

Compound I-99 was prepared from compound 97B according to methods similar to those described herein. Cpd. No. I-99 as a white solid. Yield: 99 mg; ¹H NMR (300 MHz, DMSO-d₆ with D₂O) δ 7.78 (s, 2H), 7.25 (d, J=8.5 Hz, 4H), 6.89 (d, J=8.5 Hz, 4H), 5.24 (s, 2H), 4.30-4.20 (m, 4H), 4.16-4.07 (m, 1H), 3.48 (d, J=20.2 Hz, 116H), 3.34 (dd, J=16.8, 6.0 Hz, 8H), 3.20-3.15 (m, 2H), 3.08-2.92 (m, 8H), 2.59 (t, J=7.4 Hz, 4H), 2.30 (t, J=6.4 Hz, 2H), 1.96-1.12 (m, 38H).

### Example 100: Compound I-100

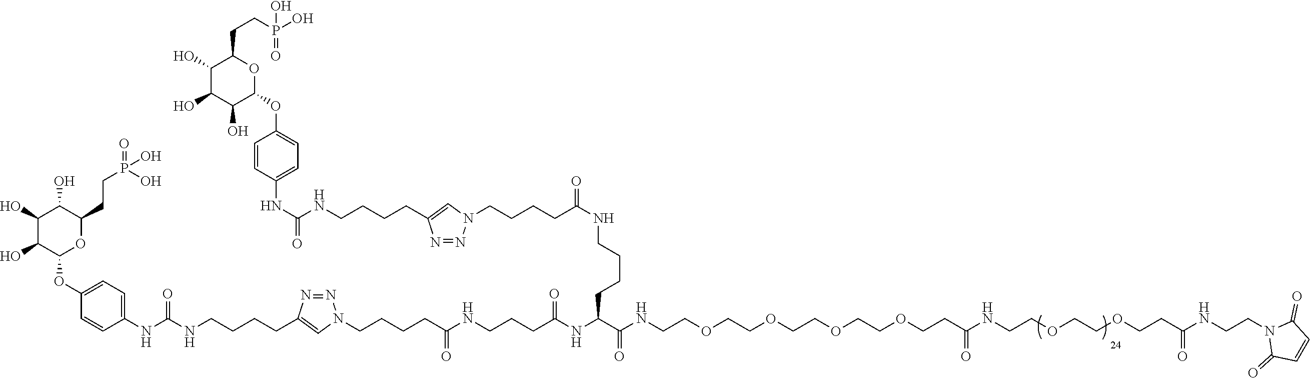

Compound I-100 was prepared from compound I-99 according to methods similar to those described herein. as a white solid. Yield: 27 mg; ¹H NMR (300 MHz, DMSO-d₆ with D₂O) δ 8.03 (br, 2H), 7.84 (s, 2H), 7.30 (d, J=8.6 Hz, 4H), 7.00-6.92 (m, 6H), 5.30 (s, 2H), 4.31 (t, J=6.9 Hz, 4H), 4.20-4.14 (m, 1H), 3.70-3.36 (m, 120H), 3.28-3.19 (m, 6H), 3.17-2.98 (m, 10H), 2.65 (t, J=7.4 Hz, 4H), 2.36 (t, J=6.3 Hz, 2H), 2.27 (t, J=6.5 Hz, 2H), 2.19-2.08 (m, 6H), 2.02-1.18 (m, 32H).

### Example 101A: Synthesis of (2-((2R,3S,4R,5S,6R)-6-(4-(3-(hex-5-yn yl)ureido)benzyl)-3,4,5-trihydroxytetrahydro-2H-pyran-2-yl)ethyl)phosphonic acid (Cpd. No. I-101)

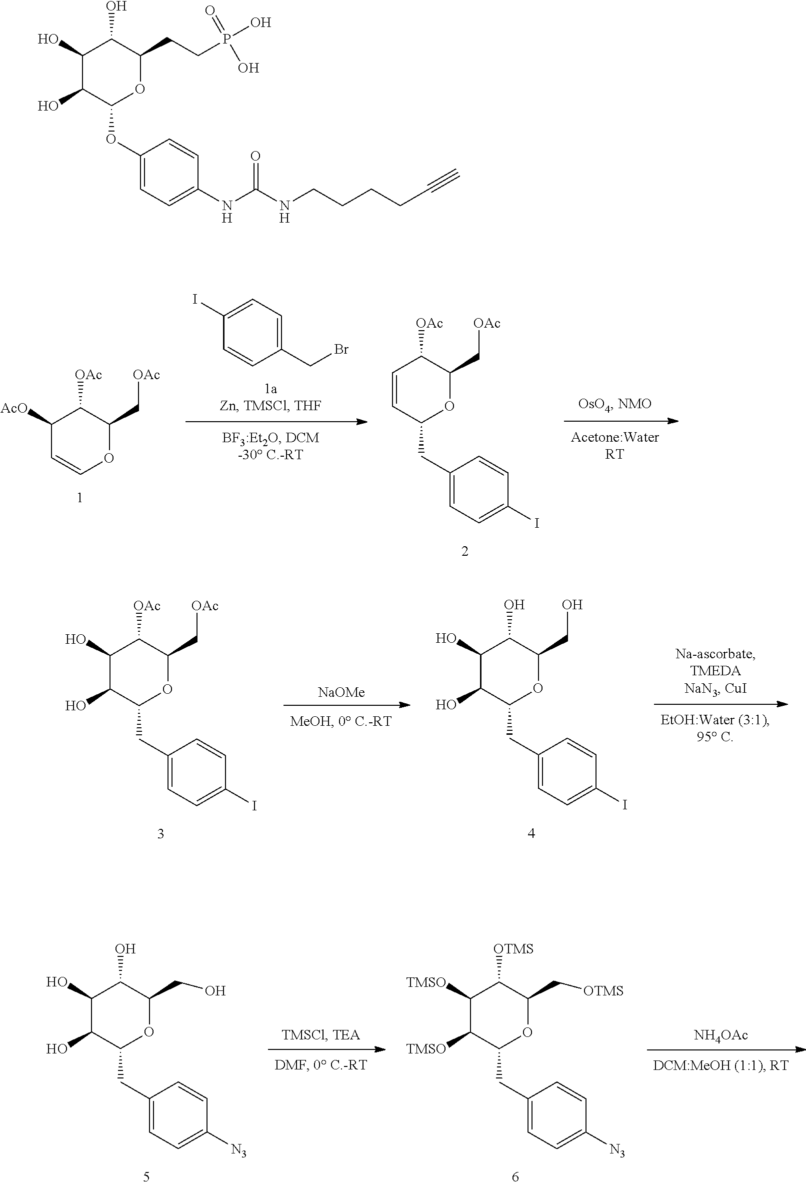

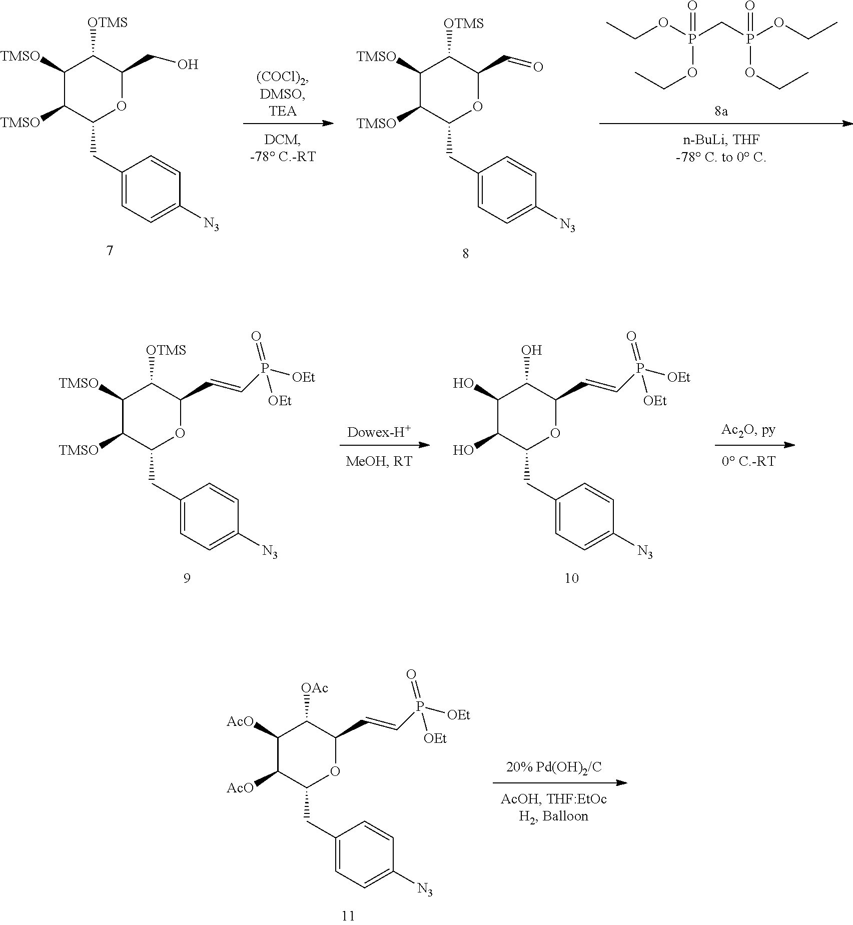

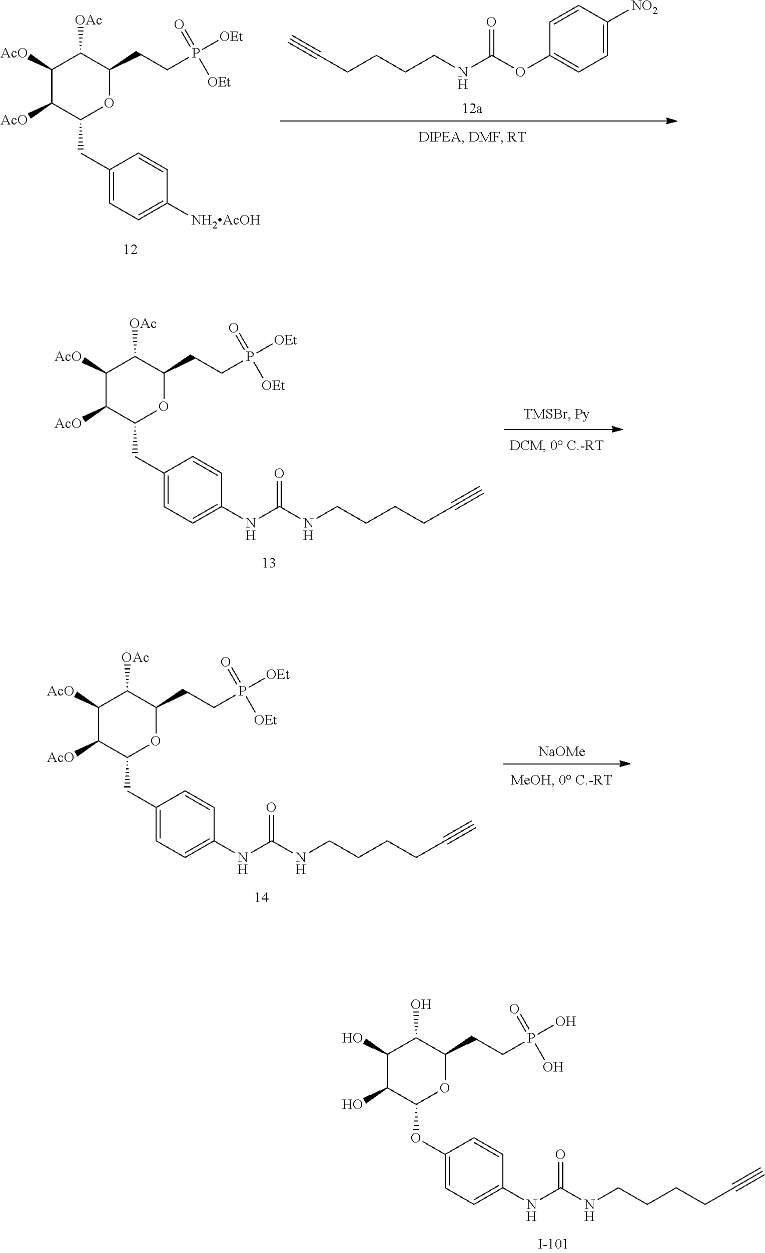

### Synthesis of ((2R,3S,6R)-3-acetoxy-6-(4-iodobenzyl)-3,6-dihydro-2H-pyran yl)methyl acetate (2)

Zinc dust (8.01 g, 2.0 eq, 123 mmol) was heated with a heat gun under vacuum for 5 min and cooled to room temperature under vacuum. Dry tetrahydrofuran (10.0 mL) and 1,2-dibromoethane (0.422 mL, 0.08 eq, 4.90 mmol) were added to zinc dust at room temperature and the resulting slurry heated to 60° C. with stirring under nitrogen for 10 min. The slurry cooled to room temperature and chlorotrimethylsilane (0.468 mL, 0.06 eq, 3.69 mmol) added to the previous slurry. The resulting slurry was then stirred for 10 more mins and cooled to 0° C. A solution of 4-iodobenzyl bromide (18.20 g, 1.0 eq, 61.3 mmol) in dry tetrahydrofuran (40.0 mL) was added dropwise, over 1 h, to the stirred suspension of activated zinc at 0° C. under argon in the dark. After addition the mixture was warmed to room temperature and allowed to settle. The zincate solution was transferred away from unreacted zinc via gastight syringe, placed into a flask purged with argon, and the solvent was removed in vacuo (bath temp 35° C.). Dry dichloromethane (40.0 mL) was added to the residue, and the solution was cooled to −30° C. under argon in the dark. A solution of (2R,3S,4R)-2-(acetoxymethyl)-3,4-dihydro-2H-pyran-3,4-diyl diacetate (10.0 g, 0.6 eq, 36.8 mmol) in dry dichloromethane (20.0 mL) was added to the zincate, followed by BF₃:OEt₂ (22.6 mL, 3.0 eq, 184 mmol). The mixture was immediately warmed to 0° C. and stirred for 15 min. The reaction mixture was warmed to room temperature, then diluted with dichloromethane (80 mL), and washed with brine (20 mL); and the organic layer was dried over sodium sulfate, filtered; and the solvent was removed in vacuo. The residue was purified by flash chromatography (ethyl acetate-light petroleum, 1:3) to afford the title compound ((2R,3S,6R)-3-acetoxy-6-(4-iodobenzyl)-3,6-dihydro-2H-pyran-2-yl)methyl acetate (2) as a colorless oil. Yield: 7.10 g (44.9%); LCMS, m/z 371.21 [M-OAc]⁺.

### Synthesis of ((2R,3S,4R,5S,6R)-3-acetoxy-4,5-dihydroxy-6-(4-iodobenzyl)tetrahydro-2H-pyran-2-yl)methyl acetate (3)

N-Methylmorpholine N-oxide (2.25 g, 1.2 eq, 19.2 mmol) and then osmium tetra-oxide (4.0 wt % in water, 10.2 mL, 0.1 eq, 1.60 mmol) were added to a stirred solution of [(2R,3S,6R)-3-(acetyloxy)-6-[(4-iodophenyl)methyl]-3,6-dihydro-2H-pyran-2-yl]methyl acetate (2, 6.90 g, 1.0 eq, 16.0 mmol) in acetone-water (5:1, 80.0 mL) at room temperature. After 24 h, TLC (ethyl acetate-light petroleum, 3:2) indicated no starting material (R_{f} 0.8) remained and a new spot generated (R_{f} 0.1). Sodium metabisulfite (0.610 g, 0.2 eq, 3.21 mmol) in water (5 mL) was added, and the mixture was stirred vigorously for 0.5 h. Ethyl acetate (50 mL) was added, and the mixture was filtered through Celite into a separating funnel and washed with brine (10 mL). The aqueous layer was extracted with ethyl acetate, and the combined organic fractions were dried over sodium sulfate, filtered and the solvent was removed in vacuo. The residue was purified by flash chromatography (eluent gradient, ethyl acetate-light petroleum, 2:1 to ethyl acetate) to afford ((2R,3S,4R,5S,6R)-3-acetoxy-4,5-dihydroxy-6-(4-iodobenzyl)tetrahydro-2H-pyran-2-yl)methyl acetate (3) as a white solid. Yield: 6.00 g (80.5%); LCMS m/z 482.13 [M+18]⁺.

### Synthesis of (2R,3S,4R,5S,6R)-2-(hydroxymethyl)-6-(4-iodobenzyl)tetrahydro-2H-pyran-3,4,5-triol (4)

((2R,3S,4R,5S,6R)-3-acetoxy-4,5-dihydroxy-6-(4-iodobenzyl)tetrahydro-2H-pyran-2-yl)methyl acetate (3, 6.00 g, 1.0 eq, 12.92 mmol) dissolved in methanol (60.0 mL) and cooled to 0° C. followed by addition of sodium methoxide (0.287 mL, 0.1 eq, 1.29 mmol, 25% w/v solution in methanol). The reaction mixture was stirred at room temperature for 15 min and TLC checked. After completion of reaction, Dowex-50w X8-Hydrogen form added up to neutral pH, the reaction mass was then filtered through sintered and concentrated in vacuo to get (2R,3S,4R,5S,6R)-2-(hydroxymethyl)-6-(4-iodobenzyl)tetrahydro-2H-pyran-3,4,5-triol as off white solid. Yield: 4.10 g (83.4%). LCMS m/z 381.18 [M+H]⁺.

### Synthesis of (2R,3S,4R,5S,6R)-2-(4-azidobenzyl)-6-(hydroxymethyl)tetrahydro-2H-pyran-3,4,5-triol (5)

A mixture of (2R,3S,4R,5S,6R)-2-(hydroxymethyl)-6-(4-iodobenzyl)tetrahydro-2H-pyran-3,4,5-triol (4, 4.0 g, 1.0 eq, 10.5 mmol), diiodocopper (1.67 g, 0.5 eq, 5.26 mmol), sodium azide (1.37 g, 2.0 eq, 21.0 mmol), [2-(dimethylamino)ethyl]dimethylamine (0.476 mL, 0.3 eq, 3.16 mmol) and sodium ascorbate (0.625 g, 0.3 eq, 3.16 mmol) in ethanol:water (50.0 mL, 7:3) in a closed flask was heated to 95° C. under argon and the progress of reaction was monitored by LCMS. After 24 h, reaction was concentrated to dryness under vacuo and the crude was dissolved in methanol, filtered through sintered glass funnel, concentrated, and dried under vacuo to afford (2R,3S,4R,5S,6R)-2-(4-azidobenzyl)-6-(hydroxymethyl)tetrahydro-2H-pyran-3,4,5-triol (5) as white solid. Yield:3.10 g (99.7%) LCMS m/z 294.57 [M−1]⁻.

### Synthesis of (((2R,3R,4R,5R,6R)-2-(4-azidobenzyl)-6-(((trimethylsilyl)oxy)methyl)tetrahydro-2H-pyran-3,4,5-triyl)tris(oxy))tris(trimethylsilane) (6)

A stirred solution of (2R,3S,4R,5S,6R)-2-(4-azidobenzyl)-6-(hydroxymethyl)tetrahydro-2H-pyran-3,4,5-triol (5, 1.0 eq, 3.0 g, 10.16 mmol) in N,N-dimethylformamide (40.0 mL) was cooled to 0° C. Then, triethylamine (6.4 eq, 288 mL, 552.0 mmol) and trimethylsilyl chloride (24.0 eq 70 mL, 2071.0 mmol) was added respectively under nitrogen atmosphere to above solution. The resulting mixture was stirred at room temperature under nitrogen for 16 h. The reaction mixture was then partitioned between ethyl acetate and water. The water layer was extracted again with ethyl acetate. The combined organic layers were dried over sodium sulfate, filtered, and purified by silica gel chromatography (0 to 5% ethyl acetate in hexane) to afford (((2R,3R,4R,5R,6R)-2-(4-azidobenzyl)-6-(((trimethylsilyl)oxy)methyl)tetrahydro-2H-pyran-3,4,5-triyl)tris(oxy))tris(trimethylsilane) (6) as white solid. Yield: 2.78 g (46.3%); LCMS m/z 584.17 [M+1]⁺.

### Synthesis of ((2R,3R,4R,5R,6R)-6-(4-azidobenzyl)-3,4,5-tris((trimethylsilyl)oxy)tetrahydro-2H-pyran-2-yl)methanol (7)

To a stirred solution of (((2R,3R,4R,5R,6R)-2-(4-azidobenzyl)-6-(((trimethylsilyl)oxy)methyl)tetrahydro-2H-pyran-3,4,5-triyl)tris(oxy))tris(trimethylsilane) (6, 1.0 eq, 2.7 g, 4.62 mmol) in mixture of DCM:MeOH (1:1, 30 mL) ammonium acetate (1.5 eq, 0.534 g, 6.93 mmol) was added at room temperature under nitrogen. The resulting mixture was stirred at room temperature under nitrogen for 16 h. The reaction mixture was then partitioned between ethyl acetate and water. The water layer was extracted again with ethyl acetate. The combined organic layers were dried over sodium sulfate, filtered, concentrated under vacuum and purified via silica gel chromatography (20-30% ethyl acetate in hexane) to afford ((2R,3R,4R,5R,6R)-6-(4-azidobenzyl)-3,4,5-tris((trimethylsilyl)oxy)tetrahydro-2H-pyran-2-yl)methanol (7) as thick syrup. Yield: 2.08 g (87%); LCMS m/z 510.13 [M−1]⁻.

### Synthesis of (2S,3R,4R,5R,6R)-6-(4-azidobenzyl)-3,4,5-tris((trimethylsilyl)oxy)tetrahydro-2H-pyran-2-carbaldehyde (8)

To a stirred solution of oxalyl chloride (1.1 eq, 0.371 mL, 4.30 mmol) in DCM (5 mL) at −78° C. was added a solution of dimethyl sulfoxide (2.2 eq, 0.611 mL, 8.60 mmol) in dichloromethane (5 mL) over 5 min. After being stirred at −78° C. for 20 min, a solution of ((2R,3R,4R,5R,6R)-6-(4-azidobenzyl)-3,4,5-tris((trimethylsilyl)oxy)tetrahydro-2H-pyran-2-yl)methanol (7, 1.0 eq, 2.0 g, 3.91 mmol) in dichloromethane (10 mL) was added to the mixture. The reaction mixture was further stirred at −78° C. for 60 min, followed by addition of triethylamine (5.0 eq, 2.75 mL, 19.5 mmol). The resulting mixture was allowed to reach room temperature over 1 h. The turbid mixture was diluted with dichloromethane and washed with water followed by brine solution. The organic layer was dried over sodium sulfate, filtered, and concentrated under high vacuum to afford (2S,3R,4R,5R,6R)-6-(4-azidobenzyl)-3,4,5-tris((trimethylsilyl)oxy)tetrahydro-2H-pyran-2-carbaldehyde (8) as light brown gel. Yield (2.4 g, Crude).which was used directly in the next step.

### Synthesis of diethyl ((E)-2-((2R,3R,4R,5R,6R)-6-(4-azidobenzyl)-3,4,5-tris((trimethylsilyl)oxy)tetrahydro-2H-pyran-2-yl)vinyl)phosphonate (9)

A stirred suspension of tetraethyl methylenebis(phosphonate) (8a, 1.5 eq, 1.96 mL, 7.06 mmol) in dry tetrahedron (50 mL) was cooled to −78° C. and added n-BuLi solution (1.5 eq, 2.94 ml, 7.06 mmol, 2.4 M in Hexane). The resulting mixture was stirred for 1 h at −78° C., then (2S,3R,4R,5R,6R)-6-(4-azidobenzyl)-3,4,5-tris((trimethylsilyl)oxy)tetrahydro-2H-pyran-2-carbaldehyde (8, 1.0 eq, 2.40 g, 4.71 mmol) in dry tetrahedron (10 mL) was added at −78° C. The bath was removed and the reaction mixture was allowed to reach room temperature and stirring continued for 12 h. A saturated aqueous solution of ammonium chloride was added and extracted with ethyl acetate. Ethyl acetate layer was washed with water followed by brine solution. The organic layer was dried over sodium sulfate, filtered and concentrated. The crude was purified by silica gel chromatography (30-40% ethyl acetate in Hexane) to afford diethyl ((E)-2-((2R,3R,4R,5R,6R)-6-(4-azidobenzyl)-3,4,5-tris((trimethylsilyl)oxy)tetrahydro-2H-pyran-2-yl)vinyl)phosphonate (9) as colorless gel. Yield (2.0 g, 65%); LCMS m/z 644.5 [M+1]⁺.

### Synthesis of diethyl ((E)-2-((2R,3S,4R,5S,6R)-6-(4-azidobenzyl)-3,4,5-trihydroxytetrahydro-2H-pyran-2-yl)vinyl)phosphonate (10)

To a stirred solution of diethyl ((E)-2-((2R,3R,4R,5R,6R)-6-(4-azidobenzyl)-3,4,5-tris((trimethylsilyl)oxy)tetrahydro-2H-pyran-2-yl)vinyl)phosphonate (9, 1.0 eq, 2.0 g, 3.11 mmol) in methanol (15 mL). was added Dowex-50W×8 (0.50 g) at room temperature under nitrogen atmosphere. The resulting mixture was stirred at room temperature for 2 h then filtered, washed with methanol and filtrate was concentrated under vacuum to afford diethyl ((E)-2-((2R,3S,4R,5S,6R)-6-(4-azidobenzyl)-3,4,5-trihydroxytetrahydro-2H-pyran-2-yl)vinyl)phosphonate (10) as off white solid. Yield: 1.10 g (83%); LC-MS; m/z, 426.47 [M−1]⁻.

### Synthesis of (2R,3R,4R,5R,6R)-2-(4-azidobenzyl)-6-((E)-2-(diethoxyphosphoryl)vinyl)tetrahydro-2H-pyran-3,4,5-triyl triacetate (11)

To a stirred solution of diethyl ((E)-2-((2R,3S,4R,5S,6R)-6-(4-azidobenzyl)-3,4,5-trihydroxytetrahydro-2H-pyran-2-yl)vinyl)phosphonate (10, 1.00 eq, 0.89 g, 2.08 mmol) in pyridine (10 mL) was added an acetic anhydride (15.0 eq, 2.95 mL, 31.2 mmol) dropwise at 0° C. under nitrogen. The cold bath was removed and the resulting mixture was stirred at room temperature under nitrogen for 16 h. The volatiles were removed on a high vacuum and the residue was partitioned between ethyl acetate and aqueous 1N-HCl. The water layer was extracted again with ethyl acetate. The combined organic layers were dried over sodium sulfate, filtered, concentrated and purified by silica gel chromatography (30% ethyl acetate in dichloromethane) to afford (2R,3R,4R,5R,6R)-2-(4-azidobenzyl)-6-((E)-2-(diethoxyphosphoryl)vinyl)tetrahydro-2H-pyran-3,4,5-triyl triacetate (11) as thick syrup. Yield: 1.0 g (93%); LC-MS, m/z 554.54 [M+1]⁺.

### Synthesis of (2R,3R,4R,5R,6R)-2-(4-aminobenzyl)-6-(2-(diethoxyphosphoryl)ethyl)tetrahydro-2H-pyran-3,4,5-triyl triacetate acetate (12)

To a stirred solution of (2R,3R,4R,5R,6R)-2-(4-azidobenzyl)-6-((E)-2-(diethoxyphosphoryl)vinyl)tetrahydro-2H-pyran-3,4,5-triyl triacetate (11, 1.00 eq, 1.0 g, 1.90 mmol) in tetrahydrofuran:ethyl acetate (1:1, 15 mL) 20% palladium hydroxide on carbon (0.50 g) and glacial acetic acid (1.5 eq, 0.162 mL, 2.83 mmol) were added at room temperature under nitrogen. The resulting mixture was stirred at room temperature under hydrogen gas pressure (10 psi) for 3 h. The reaction mixture filtered through celite bed and washed with methanol, filtrate concentrated under vacuum to afford (2R,3R,4R,5R,6R)-2-(4-aminobenzyl)-6-(2-(diethoxyphosphoryl)ethyl)tetrahydro-2H-pyran-3,4,5-triyl triacetate acetate (12) as brown sticky gel. Yield: 1.0 g (Crude); LCMS m/z 530.21 [M+1]⁺.

### Synthesis of ((2R,3R,4R,5R,6R)-2-(2-(diethoxyphosphoryl)ethyl)-6-(4-(3-(hex yn-1-yl)ureido)benzyl)tetrahydro-2H-pyran-3,4,5-triyl triacetate (13)

To a solution of (2R,3R,4R,5R,6R)-2-(4-aminobenzyl)-6-(2-(diethoxyphosphoryl)ethyl)tetrahydro-2H-pyran-3,4,5-triyl triacetate acetate (12, 1.0 eq, 1.00 g, 1.89 mmol) in N,N-dimethyl formamide (7.0 mL), N,N-diisopropylethyl amine (1.0 eq, 0.20 mL, 1.19 mmol) and 4-nitrophenyl hex-5-yn-1-ylcarbamate (5.0 eq, 1.65 mL, 9.44 mmol) in N,N-dimethyl formamide (3.0 mL) were added. The reaction mixture was stirred at room temperature for 16 h. The reaction mixture was concentrated under reduced pressure to afford the crude. which was purified by reverse phase (Aq C-18 column) column chromatography using 20-50% acetonitrile in water as eluent. The fractions were washed with ethyl acetate. The organic layer dried over anhydrous sodium sulfate, filtered and concentrated under reduced pressure to afford (2R,3R,4R,5R,6R)-2-(2-(diethoxyphosphoryl)ethyl)-6-(4-(3-(hex-5-yn-1-yl)ureido)benzyl)tetrahydro-2H-pyran-3,4,5-triyl triacetate (13) as brown sticky solid. Yield: 1.1 g (89%); LCMS m/z 653.21 [M+1]⁺.

### Synthesis of (2-((2R,3R,4R,5R,6R)-3,4,5-triacetoxy-6-(4-(3-(hex-5-yn-1-yl)ureido)benzyl)tetrahydro-2H-pyran-2-yl)ethyl)phosphonic acid (14)

To a stirred solution of (2R,3R,4R,5R,6R)-2-(2-(diethoxyphosphoryl)ethyl)-6-(4-(3-(hex-5-yn-1-yl)ureido)benzyl)tetrahydro-2H-pyran-3,4,5-triyl triacetate (13, 1.0 eq, 1.0 g, 1.53 mmol) in dichloromethane (10.0 mL), pyridine (10.0 eq, 1.35 mL, 15.32 mmol) cooled to 0° C. and bromotrimethylsilane (10.0 eq, 1.68 mL, 15.32 mmol) was added and reaction mixture was stirred at room temperature for 16 h. After completion, reaction mixture was quenched with ice water, extracted with dichloromethane. The organic layer was dried, concentrated under reduced pressure to afford off white solid. It was further washed with diethyl ether and dried to afford (2-((2R,3R,4R,5R,6R)-3,4,5-triacetoxy-6-(4-(3-(hex-5-yn-1-yl)ureido)benzyl)tetrahydro-2H-pyran-2-yl)ethyl)phosphonic acid (14) as off white solid. Yield: 0.87 g (95%); LCMS m/z 595.21 [M−1]⁻.

### Synthesis of (2-((2R,3S,4R,5S,6R)-6-(4-(3-(hex-5-yn-1-yl)ureido)benzyl)-3,4,5-trihydroxytetrahydro-2H-pyran-2-yl)ethyl)phosphonic acid (Cpd. No. I-101)

(2-((2R,3R,4R,5R,6R)-3,4,5-triacetoxy-6-(4-(3-(hex-5-yn-1-yl)ureido)benzyl)tetrahydro-2H-pyran-2-yl)ethyl)phosphonic acid (14, 0.48 g, 1.0 eq, 0.816 mmol) dissolved in methanol (10.0 mL) and cooled to 0° C. followed by addition of sodium methoxide (0.18 mL, 1.0 eq, 0.816 mmol, 25% w/v solution in methanol). The reaction stirred at room temperature for 15 min and followed by TLC. After completion of reaction, Dowex-50w×8-Hydrogen form was added until a neutral pH was obtained. The reaction was filtered through sintered glass funnel, concentrated in vacuo and purified by reverse phase prep-HPLC purification with (30-45% acetonitrile in water with 0.1% TFA buffer) to get (2-((2R,3S,4R,5S,6R)-6-(4-(3-(hex-5-yn-1-yl)ureido)benzyl)-3,4,5-trihydroxytetrahydro-2H-pyran-2-yl)ethyl)phosphonic acid (Cpd. No. I-101). Yield 0.015 g, (4%); LCMS, m/z 471.18 [M+1]⁺. ¹H NMR (400 MHz, MeOD) δ 7.27 (d, J=8.4 Hz, 2H), 7.14 (d, J=8.4 Hz, 2H), 4.03 (t, J=8.4 Hz, 1H), 3.78-3.76 (m, 2H), 3.51-3.47 (m, 2H), 3.21 (t, J=6.8 Hz, 2H), 2.95-2.89 (m, 1H), 2.85-2.80 (m, 1H), 2.24-2.21 (m, 3H), 2.09-2.07 (m, 1H), 1.76-1.74 (m, 2H), 1.68-1.62 (m, 2H), 1.60-1.57 (m, 2H), 1.56-1.47 (m, 1H).

### Example 101B: (2-((2R,3S,4R,5S,6R)-3,4,5-trihydroxy-6-((1-(15-oxo-15-(perfluorophenoxy)-3,6,9,12-tetraoxapentadecyl)-1H-1,2,3-triazol-4-yl)methyl)tetrahydro-2H-pyran-2-yl)ethyl)phosphonic acid (Cpd. No. I-101B)

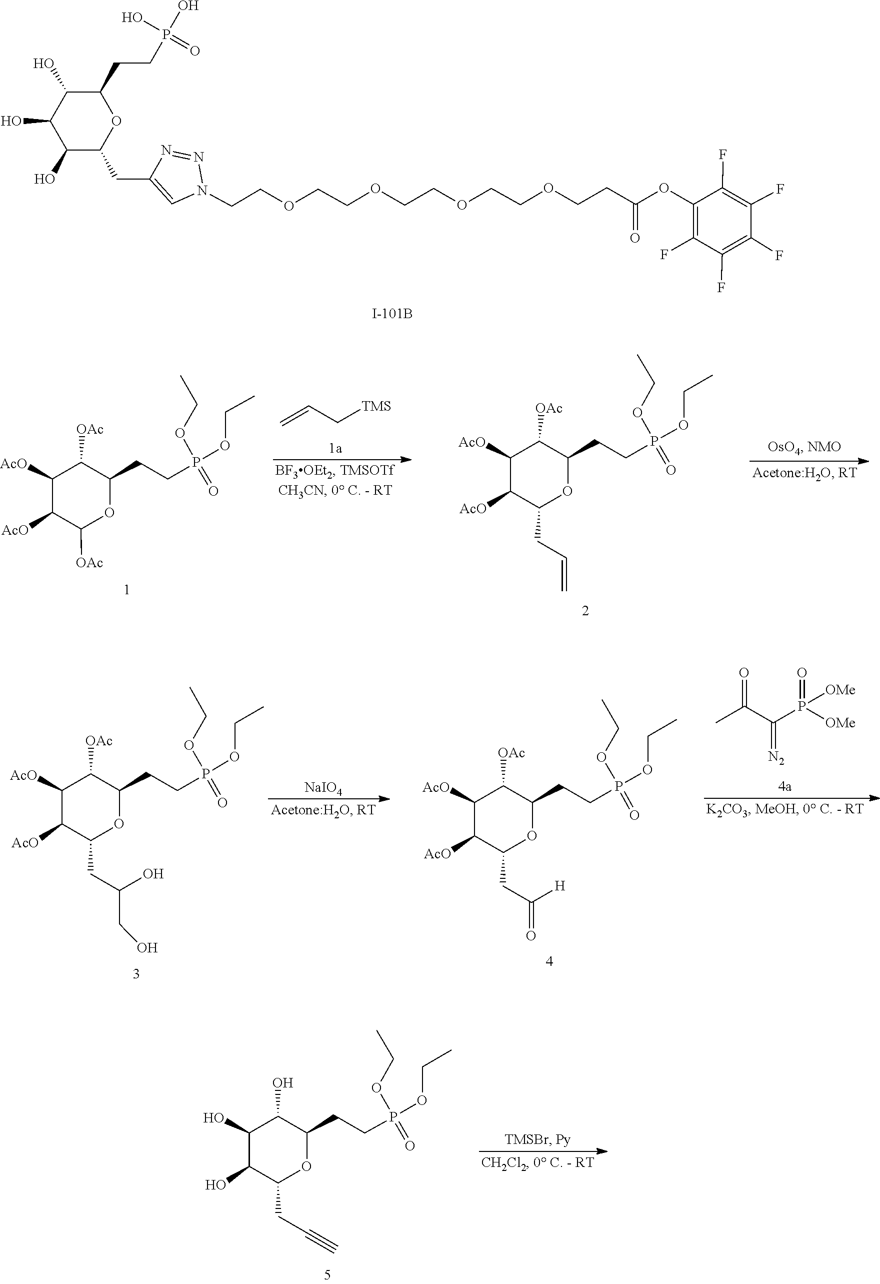

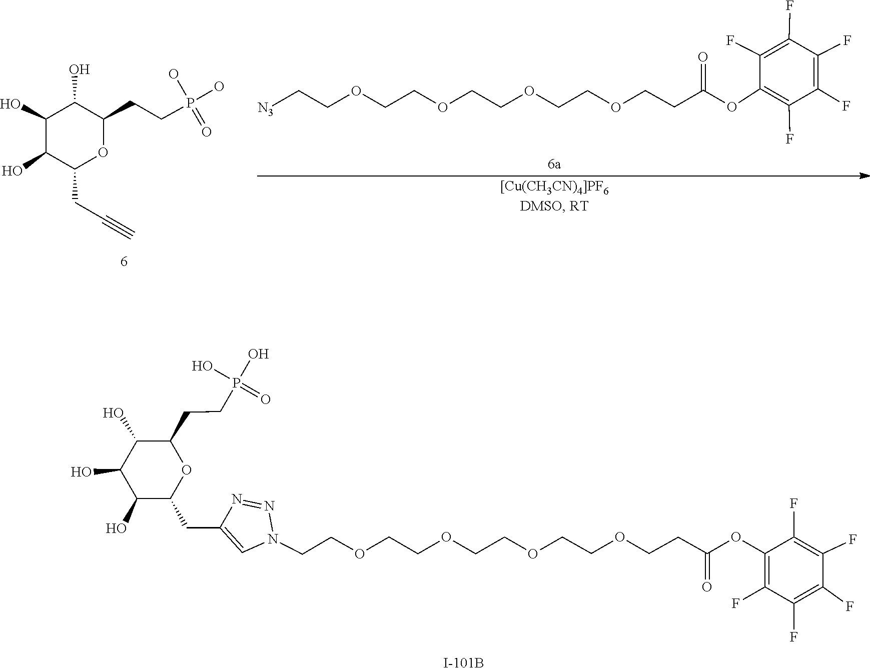 [SMILES: C#CC[C@H]1O[C@H](CCP(=O)(O)O)[C@@H](O)[C@H](O)[C@@H]1O ; [N-]=[N+]=NCCOCCOCCOCCOCCC(=O)Oc1c(F)c(F)c(F)c(F)c1F ; O=C(CCOCCOCCOCCOCCn1cc(C[C@H]2O[C@H](CCP(=O)(O)O)[C@@H](O)[C@H](O)[C@@H]2O)nn1)Oc1c(F)c(F)c(F)c(F)c1F]

### Synthesis of (2R,3R,4R,5R,6R)-2-allyl-6-(2-(diethoxyphosphoryl)ethyl)tetrahydro-2H-pyran-3,4,5-triyl triacetate (2)

To a stirred solution of ((3S,4S,5R,6R)-6-(2-(diethoxyphosphoryl)ethyl)tetrahydro-2H-pyran-2,3,4,5-tetrayl tetraacetate (1, 1.0 eq., 4.3 g, 8.91 mmol) in acetonitrile (40 mL) was added allyltrimethylsilane (1a, 4.0 eq., 5.67 mL, 35.7 mmol) followed by boron trifluoride diethyl etherate (4.0 eq., 4.4 mL, 35.7 mmol) and trimethylsilyl trifluoromethanesulfonate (0.3 eq., 0.485 mL, 2.67 mmol), sequentially at 0° C. under nitrogen atmosphere. The reaction mixture was then stirred for 12 h at room temperature. After that, reaction mixture was poured into ice-cold saturated aqueous sodium bicarbonate solution and extracted with dichloromethane. Organic part was again washed with brine, dried over anhydrous sodium sulphate, concentrated and purified by silica gel column chromatography (using 10% methanol in dichloromethane) to give (2R,3R,4R,5R,6R)-2-allyl-6-(2-(diethoxyphosphoryl)ethyl)tetrahydro-2H-pyran-3,4,5-triyl triacetate (2) as light yellow syrup. Yield: 3.48 g, 84.0%, LCMS m/z 465.0 [M+1]⁺.

### Synthesis of (2R,3R,4R,5R,6R)-2-(2-(diethoxyphosphoryl)ethyl)-6-(2,3-dihydroxypropyl)tetrahydro-2H-pyran-3,4,5-triyl triacetate (3)

N-Methylmorpholine N-oxide (1.5 eq., 0.397 g, 1.5 eq, 3.39 mmol) followed by osmium tetraoxide (0.1 eq, 1.44 mL, 0.226 mmol, 4.0 wt % in water) were added to a stirred solution of (2R,3R,4R,5R,6R)-2-allyl-6-(2-(diethoxyphosphoryl)ethyl)tetrahydro-2H-pyran-3,4,5-triyl triacetate (2, 1.0 eq, 1.05 g, 2.26 mmol) in acetone-water (5:1, 30.0 mL) at room temperature. After 2 h, TLC showed complete consumption of starting material and a lower spot generated (based on TLC observation). The mixture was extracted with ethylacetate (50 mL). The organic part was dried over anhydrous sodium sulfate, filtered and the solvent was removed in vacuo to give crude (2R,3R,4R,5R,6R)-2-(2-(diethoxyphosphoryl)ethyl)-6-(2,3-dihydroxypropyl)tetrahydro-2H-pyran-3,4,5-triyl triacetate (3) which was directly used for next step.

### Synthesis of (2R,3R,4R,5R,6R)-2-(2-(diethoxyphosphoryl)ethyl)-6-(2-oxoethyl)tetrahydro-2H-pyran-3,4,5-triyl triacetate (4)

To a stirred solution of crude (2R,3R,4R,5R,6R)-2-(2-(diethoxyphosphoryl)ethyl)-6-(2,3-dihydroxypropyl)tetrahydro-2H-pyran-3,4,5-triyl triacetate (3, 1.2 g, 2.41 mmol) in a mixture of acetone: water (2:1, 20 mL) at 0° C., was added sodium periodate (2 eq, 1.03 g, 4.81 mmol) and then allowed to stir at room temperature. After being stirred at room temperature for 2 h, the TLC showed full consumption of starting material and a less polar new spot was generated on TLC. Then ethyl acetate was added to reaction mixture and extracted with ethyl acetate. The organic part was dried over anhydrous sodium sulfate, filtered and concentrated to give crude product which was then purified by flash column chromatography using 7-10% methanol in dichloromethane to give (2R,3R,4R,5R,6R)-2-(2-(diethoxyphosphoryl)ethyl)-6-(2-oxoethyl)tetrahydro-2H-pyran-3,4,5-triyl triacetate (4) as colorless syrup. Yield: 0.91 g, 81.0%. LCMS m/z 467.1 [M+1]⁺.

### Synthesis of diethyl (2-((2R,3S,4R,5S,6R)-3,4,5-trihydroxy-6-(prop-2-yn yl)tetrahydro-2H-pyran-2-yl)ethyl)phosphonate (5)

To a solution of (2R,3R,4R,5R,6R)-2-(2-(diethoxyphosphoryl)ethyl)-6-(2-oxoethyl)tetrahydro-2H-pyran-3,4,5-triyl triacetate (4, 1.00 eq, 0.91 g, 1.95 mmol) in methanol (25.0 mL) at 0° C., were added potassium carbonate (3 eq., 0.809 g, 5.85 mmol), dimethyl (1-diazo-2-oxopropyl)phosphonate (4a, 2 eq., 0.75 g, 3.9 mmol) and reaction mixture was stirred at room temperature for 3 h. TLC showed formation of polar spot. The volatiles were then evaporated in vacuo to get a crude reaction mass which was purified by silica gel flash column chromatography using 10-12% methanol in dichloromethane to give diethyl (2-((2R,3S,4R,5S,6R)-3,4,5-trihydroxy-6-(prop-2-yn-1-yl)tetrahydro-2H-pyran-2-yl)ethyl)phosphonate (5) as colorless syrup. Yield: 0.35 g, 53.3%. LCMS m/z 337.0 [M+1]⁺.

### Synthesis of (2-((2R,3S,4R,5S,6R)-3,4,5-trihydroxy-6-(prop-2-yn-1-yl)tetrahydro-2H-pyran-2-yl)ethyl)phosphonic acid (6)

To a stirred solution of diethyl (2-((2R,3S,4R,5S,6R)-3,4,5-trihydroxy-6-(prop-2-yn-1-yl)tetrahydro-2H-pyran-2-yl)ethyl)phosphonate (5, 1.0 eq, 0.35 g, 1.04 mmol) in dichloromethane (15.0 mL), were added pyridine (10.0 eq, 0.838 mL, 10.4 mmol) and bromotrimethylsilane (10.0 eq, 1.37 mL, 10.4 mmol) at 0° C. and reaction mixture was allowed to stir at room temperature. After 16 h, volatiles were evaporated and the crude mass was purified by prep-HPLC (using 40-60% acetonitrile in water with 0.1% TFA, to elute from a C18 column). The fractions containing desired compound were collected and lyophilized to give (2-((2R,3S,4R,5S,6R)-3,4,5-trihydroxy-6-(prop-2-yn-1-yl)tetrahydro-2H-pyran-2 yl)ethyl)phosphonic acid (6) as a off-white solid. Yield: 0.101 g, 34.64% LCMS m/z 281.0 [M+1]⁺.

### Synthesis of (2-((2R,3S,4R,5S,6R)-3,4,5-trihydroxy-6-((1-(15-oxo-15-(perfluorophenoxy)-3,6,9,12-tetraoxapentadecyl)-1H-1,2,3-triazol-4-yl)methyl)tetrahydro-2H-pyran-2-yl)ethyl)phosphonic acid (Cpd. No. I-101B)

A solution of 2,3,4,5,6-pentafluorophenyl 1-azido-3,6,9,12-tetraoxapentadecan-15-oate (1.1 eq, 0.156 g, 0.342 mmol) in dimethyl sulfoxide (3 mL), (2-((2R,3S,4R,5S,6R)-3,4,5-trihydroxy-6-(prop-2-yn-1-yl)tetrahydro-2H-pyran-2 yl)ethyl)phosphonic acid (6, 1.0 eq, 0.087 g, 0.310 mmol), tetrakis(acetonitrile)copper(I) hexafluorophosphate (2.8 eq., 0.324 g, 0.869 mmol) were added and reaction mixture was stirred at room temperature for 30 min. Thereafter, acetic acid (0.5 mL) was added and reaction mixture was diluted with acetonitrile and purified by prep HPLC (23-41% acetonitrile in water with 0.1% TFA). Fractions containing the desired product were combined and lyophilized to dryness to afford (2-((2R,3S,4R,5S,6R)-3,4,5-trihydroxy-6-((1-(15-oxo-15-(perfluorophenoxy)-3,6,9,12-tetraoxapentadecyl)-1H-1,2,3-triazol-4-yl)methyl)tetrahydro-2H-pyran-2-yl)ethyl)phosphonic acid Yield: 0.101 g, 44.1%, LCMS, m/z 738.20 [M+1]⁺; ¹H NMR (400 MHz, DMSO-d₆ with D₂O exchange) δ 4.44 (t, J=5.2 Hz, 2H), 3.89-3.86 (m, 1H), 3.77-3.73 (m, 4H), 3.60-3.56 (m, 2H), 3.53-3.46 (m, 13H), 3.29-3.28 (m, 2H), 2.97 (t, J=5.6 Hz, 2H), 2.86 (d, J=7.2 Hz, 2H), 1.82 (bs, 1H), 1.57 (bs, 1H), 1.46-1.31 (m, 2H).

### Example 102: (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (Cpd. No. I-102)

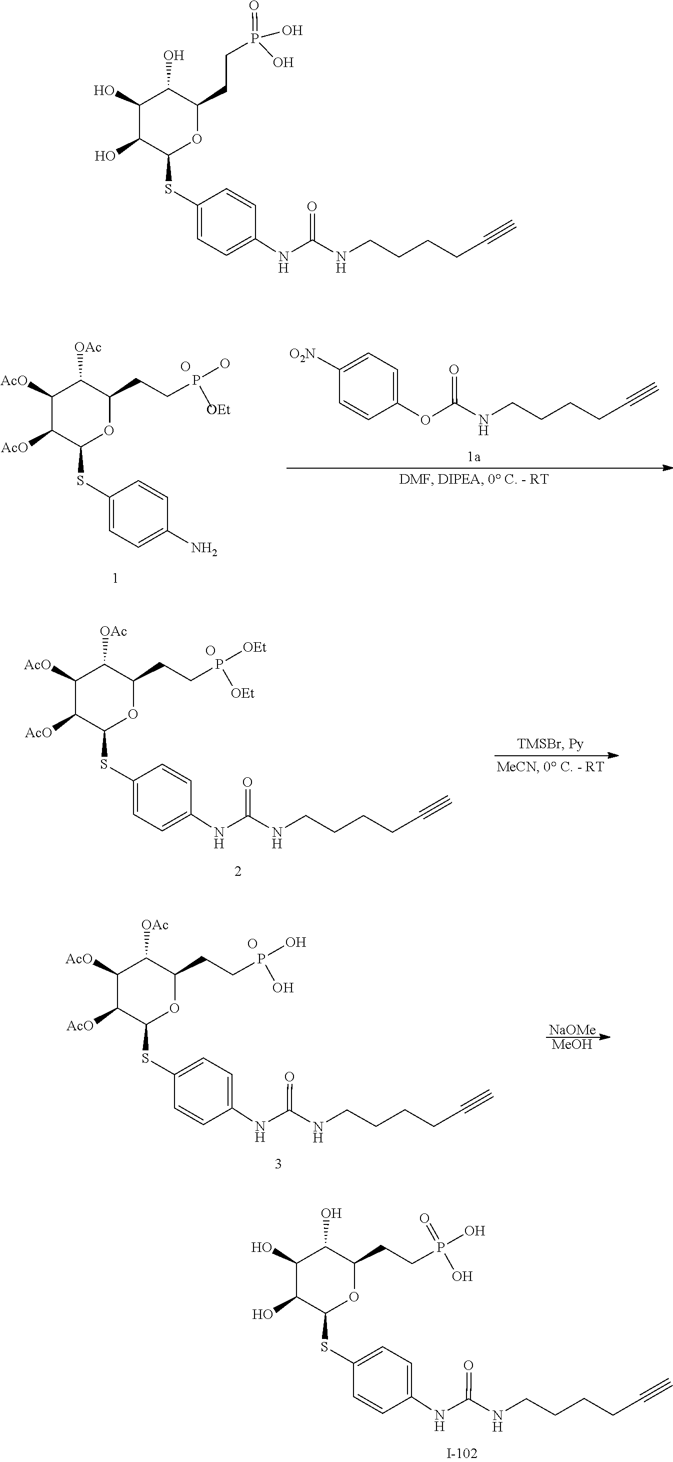

### Synthesis of (2R,3R,4S,5S,65)-2-(2-(diethoxyphosphoryl)ethyl)-6-((4-(3-(hex-5-yn-1-yl)ureido)phenyl)thio)tetrahydro-2H-pyran-3,4,5-triyl triacetate (2)

To a solution of (2S,3S,4S,5R,6R)-2-((4-aminophenyl)thio)-6-(2-(diethoxyphosphoryl)ethyl)tetrahydro-2H-pyran-3,4,5-triyl triacetate (1, 1.0 eq, 1.04 g, 1.90 mmol) in N,N-dimethyl formamide (12.0 mL), N,N-diisopropylethyl amine (2.0 eq, 0.663 mL, 3.80 mmol) and 4-nitrophenyl hex-5-yn-1-ylcarbamate (la, 2.0 eq, 0.996 g, 3.80 mmol) were added. The reaction mixture was stirred at room temperature for 16 h. The progress of reaction was monitored by LCMS. The reaction mixture was concentrated under reduced pressure to afford crude. The crude was purified by reverse phase (C-18 column) column chromatography using 20-50% acetonitrile in water as eluent. The fractions were washed with ethyl acetate. The organic layer dried over anhydrous sodium sulphate, filtered and concentrated under reduced pressure to afford (2R,3R,4S,5S,6S)-2-(2-(diethoxyphosphoryl)ethyl)-6-((4-(3-(hex-5-yn-1-yl)ureido)phenyl)thio)tetrahydro-2H-pyran-3,4,5-triyl triacetate (2) as brown sticky solid. Yield: 0.65 g (52.5%) LCMS m/z. 671.22 [M+1]⁺.

### Synthesis of (2-((2R,3R,4S,5S,6S)-3,4,5-triacetoxy-6-((4-(3-(hex-5-yn-1-yl)ureido)phenyl)thio)tetrahydro-2H-pyran-2-yl)ethyl)phosphonic acid (3)

To a stirred solution of (2R,3R,4S,5S,6S)-2-(2-(diethoxyphosphoryl)ethyl)-6-((4-(3-(hex-5-yn-1-yl)ureido)phenyl)thio)tetrahydro-2H-pyran-3,4,5-triyl triacetate (2, 1.0 eq, 0.25 g, 0.373 mmol) in dichloromethane (8.0 mL), pyridine (10.0 eq, 0.30 mL, 3.73 mmol) cooled to 0° C. and bromotrimethylsilane (10.0 eq, 0.49 mL, 3.73 mmol) was added and reaction mixture was stirred at room temperature for 16 h. After completion, reaction mixture was quenched with ice water, extracted with dichloromethane. The organic layer separated, dried over anhydrous sodium sulfate, filtered and concentrated under reduced pressure to get off-white solid. It was further washed with diethyl ether and dried to afford (2-((2R,3R,4S,5S,6S)-3,4,5-triacetoxy-6-((4-(3-(hex-5-yn-1-yl)ureido)phenyl)thio)tetrahydro-2H-pyran-2-yl)ethyl)phosphonic acid (3) as off white solid. Yield: 0.16 g (69.8%) LCMS m/z. 614.93 [M+1]⁺.

### Synthesis of (2-((2R,3S,4S,5S,65)-6-((4-(3-(hex-5-yn-1-yl)ureido)phenyl)thio)-3,4,5-trihydroxytetrahydro-2H-pyran-2-yl)ethyl)phosphonic acid (Cpd. No I-102)

To the stirred solution of (2-((2R,3R,4S,5S,6S)-3,4,5-triacetoxy-6-((4-(3-(hex-5-yn-1-yl)ureido)phenyl)thio)tetrahydro-2H-pyran-2-yl)ethyl)phosphonic acid (3, 1.0 eq, 0.08 g, 0.142 mmol) in methanol (3 mL), sodium methoxide 25% w/v in methanol (7.0 eq, 0.21 mL, 0.991 mmol) was added drop-wise to this solution and reaction mixture was allowed to stir at room temperature. The progress of the reaction was monitored by LCMS. After 2 h, reaction mixture was neutralized with Dowex-hydrogen form (200-400 mesh) (up to pH-7). The reaction mixture was filtered, concentrated under reduced pressure to get crude product. The crude was purified by prep-HPLC eluting from C18 column with 50-80% acetonitrile in water with 0.1% TFA. Fractions containing the desired product were combined and lyophilized to dryness to afford (2-((2R,3S,4S,5S,6S)-6-((4-(3-(hex-5-yn-1-yl)ureido)phenyl)thio)-3,4,5-trihydroxytetrahydro-2H-pyran-2-yl)ethyl)phosphonic acid (Cpd. No I-102) as white solid. Yield: 0.016 g, 23.1%; LC-MS m/z. 489.17 [M+1]⁺. ¹H NMR (400 MHz, DMSO-d₆) δ 8.47 (s, 1H), 7.34 (d, J=8.8 Hz, 2H), 7.25 (d, J=8.4 Hz, 2H), 6.16 (t, J=5.6 Hz, 1H), 4.93 (bs, 1H), 4.78 (s, 2H), 3.81 (s, 1H), 3.31 (dd, J=3.2, 9.2 Hz, 1H), 3.22 (t, J=9.2 Hz, 1H), 3.08 (dd, J=6.0, 11.6 Hz, 2H), 3.02-2.97 (m, 1H), 2.77 (t, J=2.8 Hz, 1H), 2.20-2.16 (m, 2H), 2.07-1.99 (m, 1H), 1.78-1.67 (m, 1H). 1.54-1.41 (m, 6H).

### Example 103: Synthesis of 2-[(2R,3S,4S,5S,6R)-6-[4-[4-[1-[2-[2-[2-[2-[3-[[4-[4-(2-cyanoethynyl)anilino]-4-oxo-butyl]amino]-3-oxo-propoxy]ethoxy]ethoxy]ethoxy]ethyl]triazol-4-yl]butylcarbamoylamino]phenoxy]-3,4,5-trihydroxy-tetrahydropyran-2-yl]ethylphosphonic acid (I-103)

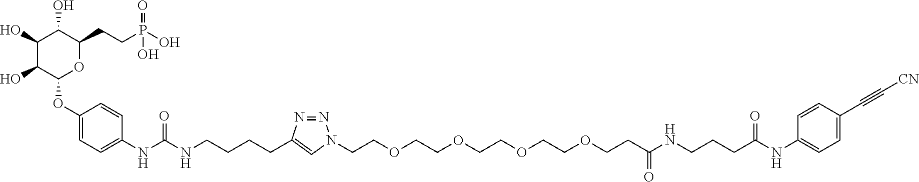 [SMILES: N#CC#Cc1ccc(NC(=O)CCCNC(=O)CCOCCOCCOCCOCCn2cc(CCCCNC(=O)Nc3ccc(O[C@H]4O[C@H](CCP(=O)(O)O)[C@@H](O)[C@H](O)[C@@H]4O)cc3)nn2)cc1]

To a N₂ sparged glass vial was added a solution of 4-[3-[2-[2-[2-(2-azidoethoxy)ethoxy]ethoxy]ethoxy]propanoylamino]-N-[4-(2-cyanoethynyl)phenyl]butanamide (1.05 eq, 20.0 mg, 0.0400 mmol) in NMP (1 mL) followed by Tetrakis(acetonitrile)copper(I) hexafluorophosphate (2.50 eq, 35.5 mg, 0.0953 mmol). The resulting clear yellow solution was capped and stirred at room temperature for 30 min. LCMS analysis found reaction to be complete. The reaction mixture was diluted with mixture of NMP, ethanol, and acetic acid, filtered, and purified via preparatory HPLC (15-65% acetonitrile in water with 0.1% TFA) Big Prep, one 30 min run, prod came off at 44%, Fractions containing the desired product were combined and lyophilized to dryness to afford the desired product 2-[(2R,3S,4S,5S,6R)-6-[4-[4-[1-[2-[2-[2-[2-[3-[[4-[4-(2-cyanoethynyl)anilino]-4-oxo-butyl]amino]-3-oxo-propoxy]ethoxy]ethoxy]ethoxy]ethyl]triazol-4-yl]butylcarbamoylamino]phenoxy]-3,4,5-trihydroxy-tetrahydropyran-2-yl]ethylphosphonic acid as a white solid. Yield: 19 mg, 48%. ¹H NMR (300 MHz, DMSO) δ 10.36 (s, 1H), 8.25 (s, 1H), 7.90 (d, J=5.5 Hz, 1H), 7.82 (s, 1H), 7.74 (s, 4H), 7.29 (d, J=9.0 Hz, 2H), 6.91 (d, J=9.0 Hz, 2H), 6.07 (s, 1H), 5.26 (d, J=1.8 Hz, 1H), 4.46 (t, J=5.2 Hz, 2H), 3.95-3.75 (m, 2H), 3.75-3.55 (m, 87H), 3.49 (d, J=2.3 Hz, 1H), 3.32 (d, J=6.7 Hz, 2H), 3.08 (t, J=6.0 Hz, 4H), 2.63 (t, J=7.4 Hz, 2H), 2.33 (dt, J=17.6, 6.9 Hz, 4H), 1.71 (p, J=6.9 Hz, 2H), 1.65-1.55 (m, 1H), 1.47 (d, J=7.8 Hz, 2H). LC-MS m/z 974 [M+1]⁺.

### Example 104: Synthesis of 2-[(2R,3S,4S,5S,6R)-3,4,5-trihydroxy-6-[4-[4-[1-[2-[2-[2-[2-[3-oxo-3-(2,3,4,5,6-pentafluorophenoxy)propoxy]ethoxy]ethoxy]ethoxy]ethyl]triazol-4-yl]butylcarbamothioylamino]phenoxy]tetrahydropyran-2-yl]ethanesulfonic acid (I-104)

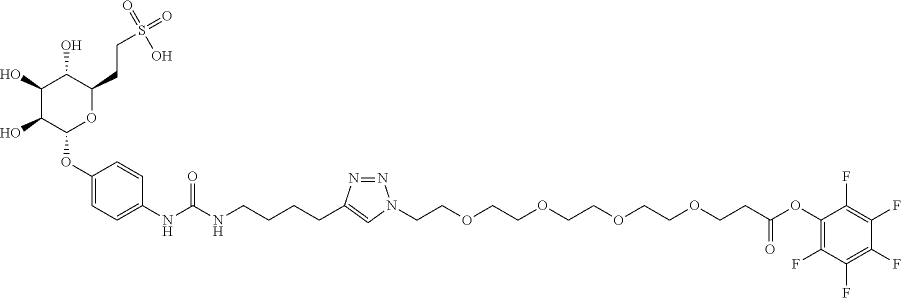 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCS(=O)(=O)O)[C@@H](O)[C@H](O)[C@@H]2O)cc1]

To a N₂ sparged glass vial was added 2-[(2R,3S,4S,5S,6R)-6-[4-(hex-5-ynylcarbamothioylamino)phenoxy]-3,4,5-trihydroxy-tetrahydropyran-2-yl]ethanesulfonic acid (1.00 eq, 11.0 mg, 0.0225 mmol) with a stirbar. To the vial was added a solution of azido-PEG4-PFP ester (1.26 eq, 13.0 mg, 0.0284 mmol) in NMP (2 mL) followed by Tetrakis(acetonitrile)copper(I) hexafluorophosphate (2.50 eq, 21.0 mg, 0.0563 mmol). The resulting clear yellow solution was capped and stirred at room temperature for 30 min. LCMS analysis found reaction to be complete. The reaction mixture was diluted with mixture of NMP, ethanol, and acetic acid, filtered, and purified via preparatory HPLC (15-65% acetonitrile in water with 0.1% TFA). Fractions containing the desired product were combined and lyophilized to dryness to afford the desired product 2-[(2R,3S,4S,5S,6R)-3,4,5-trihydroxy-6-[4-[4-[1-[2-[2-[2-[2-[3-oxo-3-(2,3,4,5,6-pentafluorophenoxy)propoxy]ethoxy]ethoxy]ethoxy]ethyl]triazol-4-yl]butylcarbamothioylamino]phenoxy]tetrahydropyran-2-yl]ethanesulfonic acid as a white solid. Yield: 9.5 mg, 42% yield. ¹H NMR (300 MHz, DMSO) δ 9.28 (s, 1H), 7.84 (s, 1H), 7.58 (s, 1H), 7.24 (d, J=8.3 Hz, 2H), 6.97 (d, J=8.4 Hz, 2H), 5.31 (s, 1H), 4.46 (t, J=5.2 Hz, 2H), 3.77 (q, J=6.2, 5.7 Hz, 6H), 3.55-3.40 (m, 16H), 3.33 (q, J=7.8, 6.1 Hz, 2H), 3.02 (t, J=5.9 Hz, 2H), 2.62 (d, J=7.0 Hz, 2H), 2.10 (q, J=14.4, 14.0 Hz, 2H), 1.58 (s, 5H). LC-MS m/z 947 [M+1]⁺.

### ASGPR LIGAND-LINKER EXAMPLES

### ASGPR Example 105

### Synthesis of [(2R,3R,4R,5R,6R)-3,4-bis(acetyloxy)-6-(but-3-yn-1-yloxy)-5-acetamidooxan-2-yl]methyl acetate (Intermediate A)

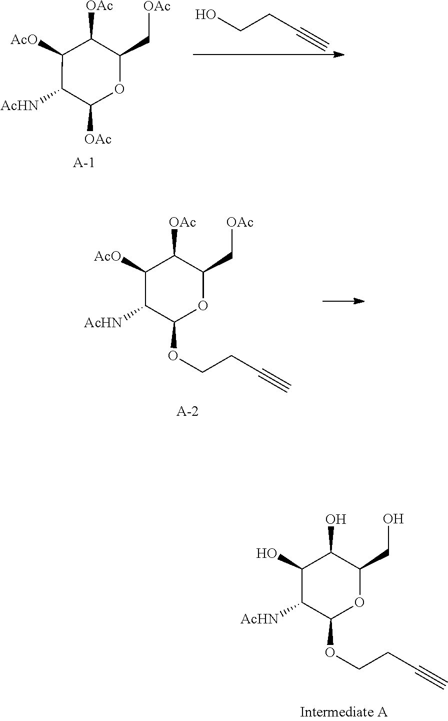

To an activated 4 Å molecular sieves (5.0 g) and [(2R,3R,4R,5R,6S)-3,4,6-tris(acetyloxy)-5-acetamidooxan-2-yl]methyl acetate (A-1) (5.0 g, 12.8 mmol), was added dichloromethane (50 mL) and stirred at room temperature for 5 min followed by addition of but-3-yn-1-ol (2.92 mL, 3.0 eq., 38.5 mmol). Stirred the reaction mixture for 10 min at room temperature and then cooled to 0° C. Diethyl trifluoroborinate (4.75 mL, 38.5 mmol) added dropwise to above reaction mixture and again stirred for 10 min at room temperature followed by 5 h refluxing at 51° C. TLC checked for the completion of reaction and triethylamine added to quench the diethyl trifluoroborinate (up to neutral pH) and filtered through celite bed followed by concentration on rotary evaporator. Obtained thick residue was purified by silica gel column purification with 60-75% ethyl acetate in dichloromethane as eluent that afforded Intermediate A-2 as an off white foam. Yield: 4.50 g, 87%; R_{f}=0.45 (7.5% methanol in dichloromethane); LC-MS m/z 400.0 [M+1]⁺; ¹H NMR (400 MHz, CDCl₃) δ 5.44 (d, J=8.6 Hz, 1H), 5.35 (d, J=7.0 Hz, 1H), 5.30 (dd, J=11.2, 3.0 Hz, 1H), 4.79 (d, J=8.2 Hz, 1H), 4.14-4.09 (m, 2H), 3.99-3.90 (m, 3H), 3.71-3.65 (m, 1H), 2.49-2.47 (m, 2H), 2.14 (s, 3H), 2.05 (s, 3H), 2.00 (s, 3H), 1.96 (s, 3H).

Intermediate A-2 (7.8 g, 17.5 mmol) was dissolved in methanol (50 mL) and cooled to 0° C. Sodium methoxide 25% w/v (2.48 mL, 11.3 mmol) in methanol added drop-wise to this solution and reaction maintained at room temperature for 3 h. TLC Checked and after completion of reaction 1N HCl was added drop-wise to quench the sodium methoxide. Methanol evaporated and obtained residue was washed with diethyl ether (30 mL×4). The crude residue obtained was purified with prep-HPLC (5-20% acetonitrile in water with 0.1% TFAH) to afford Intermediate A as a white solid. Yield: 2.6 g, 84%; LC-MS m/z 274.0 [M+1]⁺; ¹H NMR (400 MHz, D₂O) δ 4.58 (d, J=8.4 Hz, 1H), 3.97-3.86 (m, 3H), 3.82-3.73 (m, 5H), 2.49-2.44 (m, 2H), 2.04 (s, 3H).

### ASGPR Example 106

### Synthesis of N-((2R,3R,4R,5R,6R)-6-((but-3-yn-1-yloxy)methyl)-2,4,5-trihydroxytetrahydro-2H-pyran-3-yl)acetamide (Intermediate B)

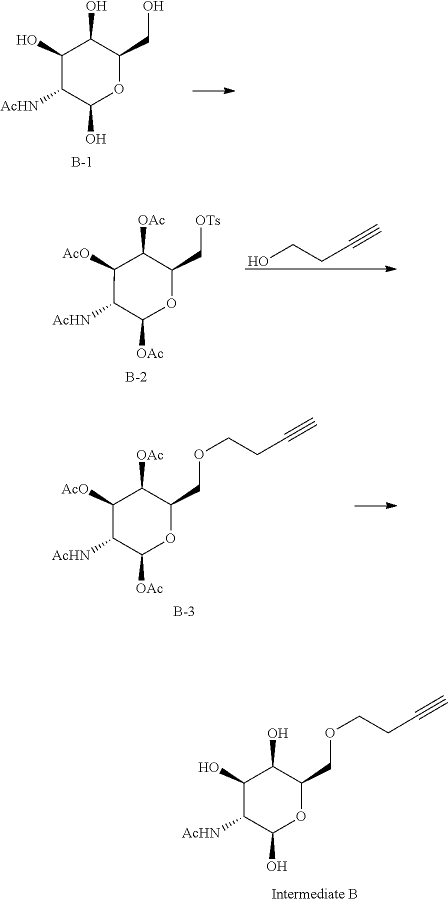

A solution of p-toluenesulfonyl choride (1.1 eq.) in dichloromethane is added slowly to a stirred solution of N-((2R,3R,4R,5R,6R)-2,4,5-trihydroxy (hydroxymethyl)tetrahydro-2H-pyran-3-yl)acetamide (B-1) (1 eq.) in dichloromethane at 0° C. The reaction mixture is warmed to room temperature and monitored by LC-MS to indicate complete formation of the desired primary alcohol tosylate. Pyridine (3.5 eq.) is added followed by acetic anhydride (3.1 eq.). The reaction mixture is stirred at room temperature and monitored by LC-MS to indicate complete formation of Intermediate B-2, which is isolated by silica gel chromatography. Sodium hydride (1.1 eq.) is added to a stirred solution of but-3-yn-1-ol (1.1 eq.) in tetrahydrofuran at 0° C. After stirring at 0° C. for 10 min a solution of Intermediate B-2 (1 eq.) in tetrahydrofuran is added. The resulting mixture is warmed to room temperature and monitored by LC-MS to indicate complete formation of Intermediate B-3, which is isolated by silica gel chromatography. Sodium methoxide in methanol (3 eq.) is added to a stirred solution of Intermediate B-3 (1 eq.) in methanol at 0° C. The resulting mixture is stirred at 0° until LC-MS indicates complete conversion to Intermediate B, which is isolated by reverse phase chromatography.

### ASGPR Example 107

### Synthesis of Triavalent GaINAc Ligand A Periluorophenyl Ester (Compound I-107)

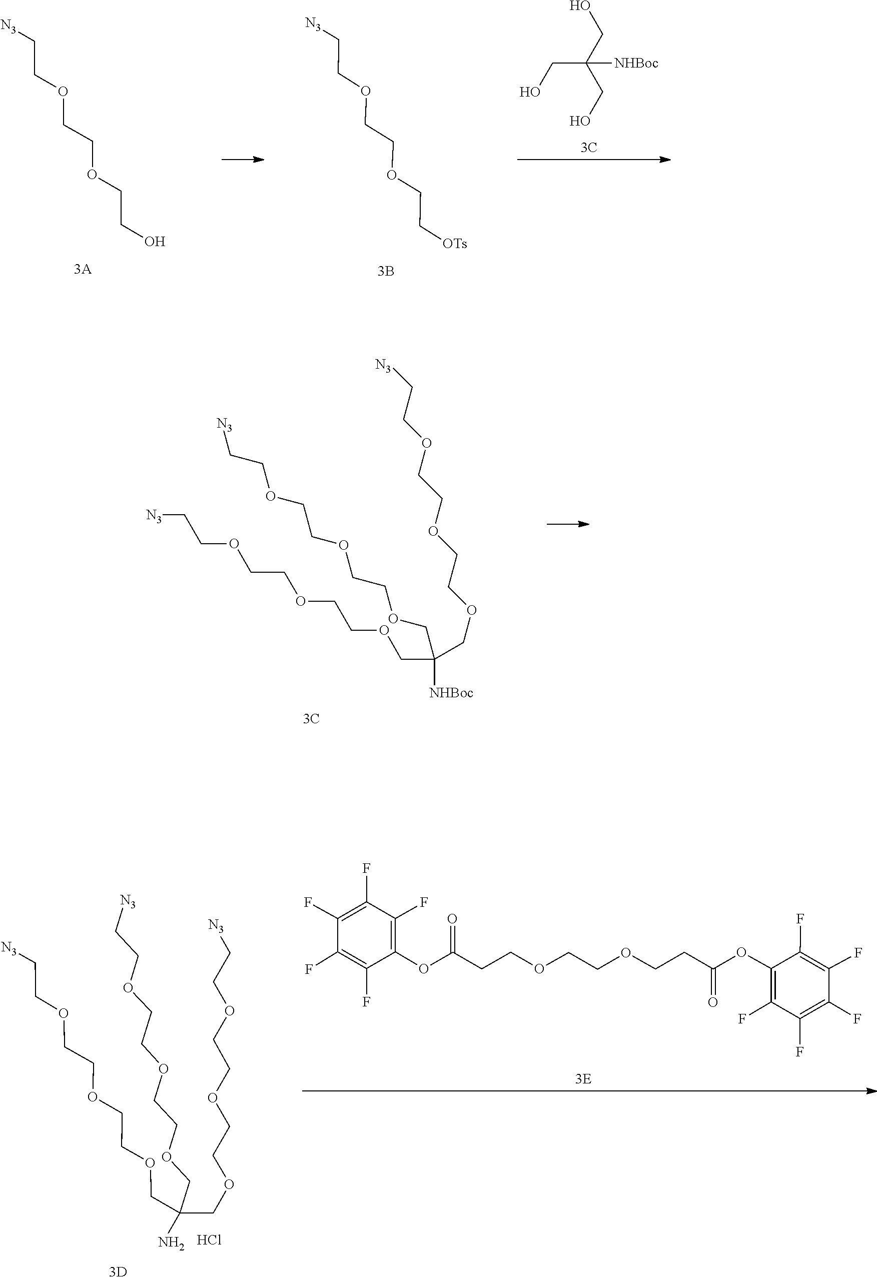

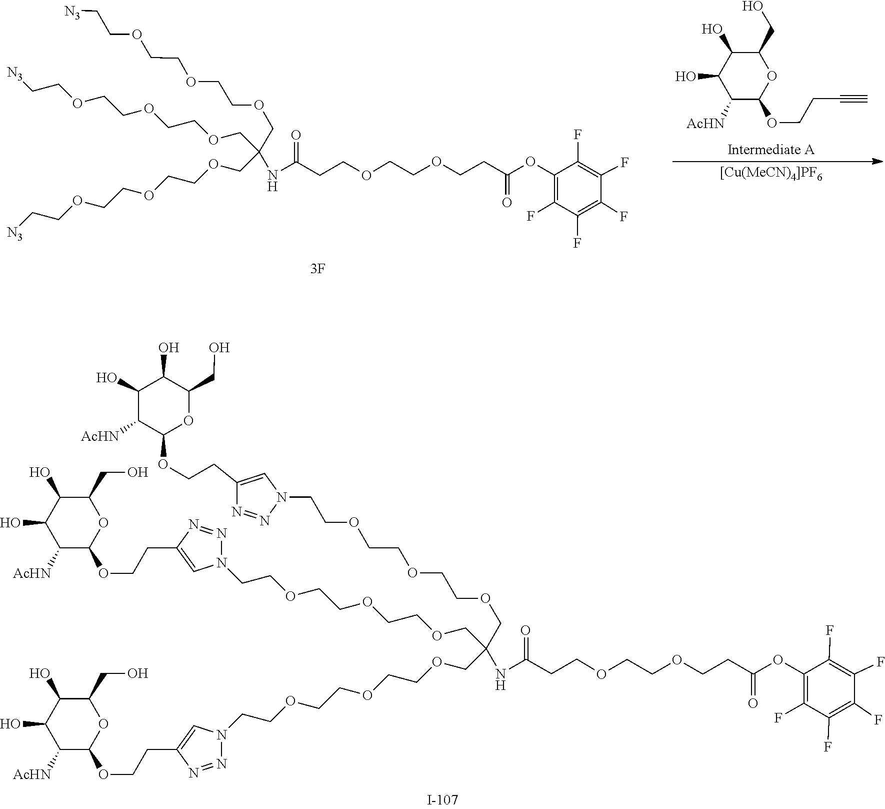 [SMILES: [N-]=[N+]=NCCOCCOCCOCC(COCCOCCOCCN=[N+]=[N-])(COCCOCCOCCN=[N+]=[N-])NC(=O)CCOCCOCCC(=O)Oc1c(F)c(F)c(F)c(F)c1F ; C#CCCO[C@@H]1O[C@H](CO)[C@H](O)[C@H](O)[C@H]1NC(C)=O ; CC(=O)N[C@H]1[C@H](OCCc2cn(CCOCCOCCOCC(COCCOCCOCCn3cc(CCO[C@@H]4O[C@H](CO)[C@H](O)[C@H](O)[C@H]4NC(C)=O)nn3)(COCCOCCOCCn3cc(CCO[C@@H]4O[C@H](CO)[C@H](O)[C@H](O)[C@H]4NC(C)=O)nn3)NC(=O)CCOCCOCCC(=O)Oc3c(F)c(F)c(F)c(F)c3F)nn2)O[C@H](CO)[C@H](O)[C@@H]1O]

A solution of p-toluenesulfonyl chloride (1.1 eq.) in dichloromethane is added to a stirred solution of 2-(2-(2-azidoethoxy)ethoxy)ethan-1-ol (3A) (1 eq.) and pyridine (1.2 eq.) in dichloromethane. The resulting mixture is stirred at room temperature and monitored by LC-MS to indicate complete formation of Compound 3B, which is isolated by silica gel chromatography. Sodium hydride is added to a stirred mixture of tert-butyl (1,3-dihydroxy (hydroxymethyl)propan-2-yl)carbamate (3C) (1 eq.) and Compound 3B (3.3 eq.) in THF at −78° C. The cold bath is removed and the resulting mixture is stirred at room temperature until LC-MS indicates complete conversion to Compound 3C, which is isolated by silica gel chromatography. HCl in diethyl ether (3 eq.) is added to a stirred solution of tert-Compound 3C (1 eq.) in dichloromethane at room temperature. The resulting mixture is stirred at room temperature until LC-MS indicates complete conversion and then volatiles are removed on a rotary evaporator to afford Compound 3D. Diisopropylethylamine (2 eq.) is added to a stirred solution of Compound 3D (1 eq.) in dichloromethane at room temperature. Bis(perfluorophenyl) 3,3′-(ethane-1,2-diylbis(oxy))dipropionate (3E) (1.1 eq.) is added and the resulting mixture is stirred at room temperature until LC-MS indicates complete conversion to Compound 3F, which is isolated by silica gel chromatography. Compound 3F (1 eq.) and Intermediate A (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-107, which is purified via reverse-phase preparatory HPLC followed by lyophilization.

### ASGPR Example 108

### Synthesis of Trivalent GaINAc Ligand B Periluorophenyl Ester (Compound I-108)

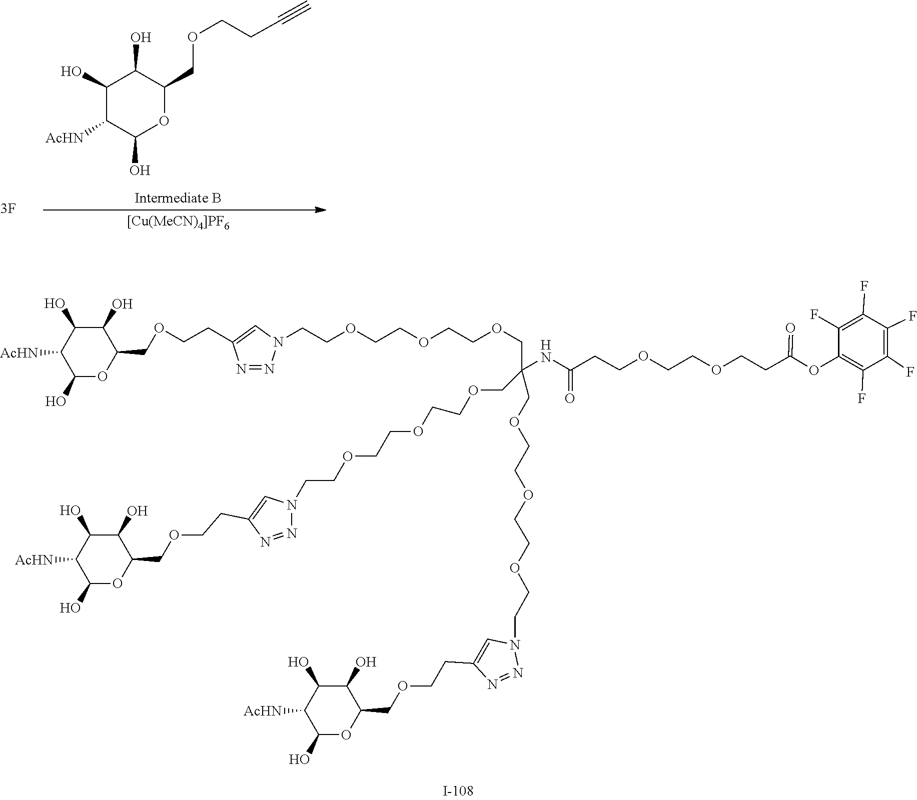 [SMILES: C#CCCOC[C@H]1O[C@@H](O)[C@H](NC(C)=O)[C@@H](O)[C@H]1O ; CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O]

Compound 3F (1 eq.) and Intermediate B (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-108, which is purified via reverse-phase preparatory HPLC followed by lyophilization.

### ASGPR Example 109

### Synthesis of Divalent GaINAc Ligand A Periluorophenyl Ester (Compound I-109)

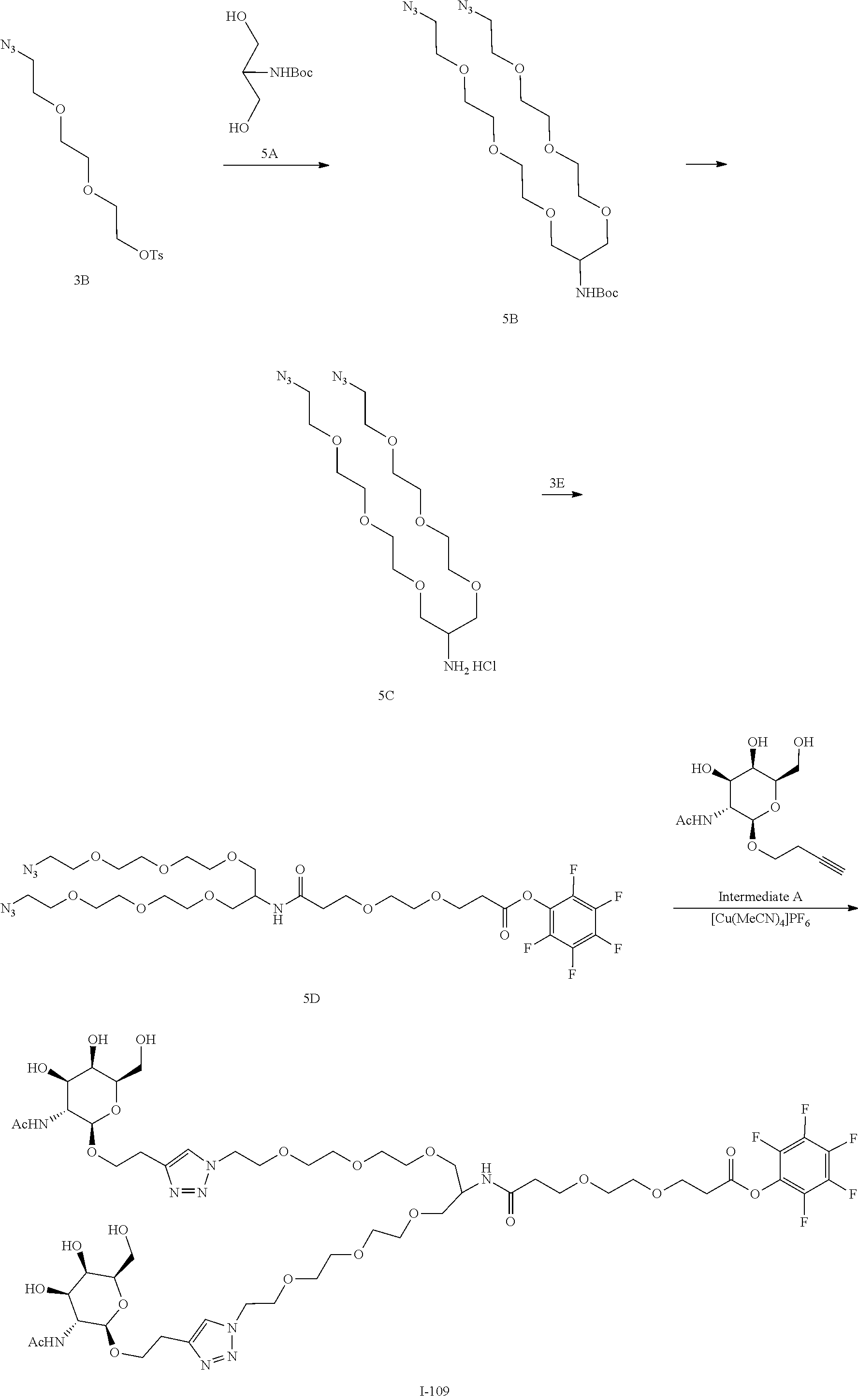

Sodium hydride is added to a stirred mixture of tert-butyl (1,3-dihydroxypropan-2-yl)carbamate (5A) (1 eq.) and Compound 3B (3.3 eq.) in THF at −78° C. The cold bath is removed and the resulting mixture is stirred at room temperature until LC-MS indicates complete conversion to Compound 5B, which is isolated by silica gel chromatography. HCl in diethyl ether (3 eq.) is added to a stirred solution of Compound 5B (1 eq.) in dichloromethane at room temperature. The resulting mixture is stirred at room temperature until LC-MS indicates complete conversion and then volatiles are removed on a rotary evaporator to afford Compound 5C. Diisopropylethylamine (2 eq.) is added to a stirred solution of Compound 5C (1 eq.) in dichloromethane at room temperature. Bis(perfluorophenyl) 3,3′-(ethane-1,2-diylbis(oxy))dipropionate (Compound 3E) (1.1 eq.) is added and the resulting mixture is stirred at room temperature until LC-MS indicates complete conversion to Compound 5D, which is isolated by silica gel chromatography. Compound 5D (1 eq.) and Intermediate A (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-5, which is purified via reverse-phase preparatory HPLC followed by lyophilization.

Following the above synthesis, 41 mg of Compound I-109 was obtained. LC-MS m/z 1336.7 [M+1]+; 1HNMR (400 MHz, D2O) d 7.87 (s, 2H), 4.65-4.61 (m, 4H), 4.47 (d, J=8.0 Hz, 2H), 4.23-4.11 (m, 2H), 4.01-3.91 (m, 10H), 3.88-3.82 (m, 10H), 3.81 (s, 1H), 3.79-3.77 (m, 4H), 3.76-3.73 (m, 12H), 3.72-3.68 (m, 14H). 3.63-3.55 (m, 6H), 3.09 (t, J=6.0 Hz, 2H), 3.00 (t, J=6.4 Hz, 4H), 1.88 (s, 6H).

### ASGPR EXAMPLE 110

### Synthesis of Divalent GaINAc Ligand B Periluorophenyl Ester (Compound I-106)

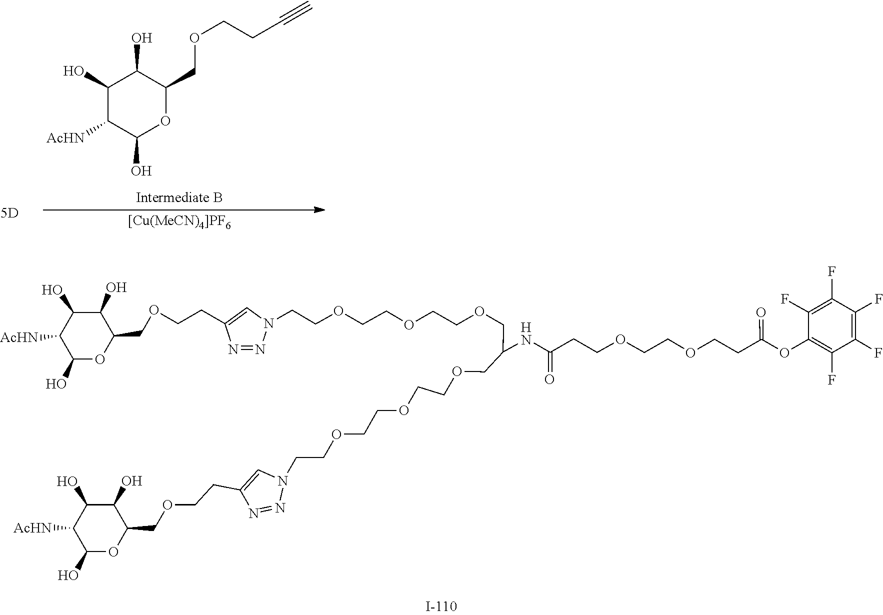 [SMILES: C#CCCOC[C@H]1O[C@@H](O)[C@H](NC(C)=O)[C@@H](O)[C@H]1O ; CC(=O)N[C@@H]1[C@@H](O)[C@@H](O)[C@@H](COCCc2cn(CCOCCOCCOCC(COCCOCCOCCn3cc(CCOC[C@H]4O[C@@H](O)[C@H](NC(C)=O)[C@@H](O)[C@H]4O)nn3)NC(=O)CCOCCOCCC(=O)Oc3c(F)c(F)c(F)c(F)c3F)nn2)O[C@H]1O]

Compound 5D (1 eq.) and Intermediate B (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-110, which is purified via reverse-phase preparatory HPLC followed by lyophilization.

### ASGPR Example 111

### Synthesis of Divalent GaINAc Ligand A Periluorophenyl Ester (Compound I-111)

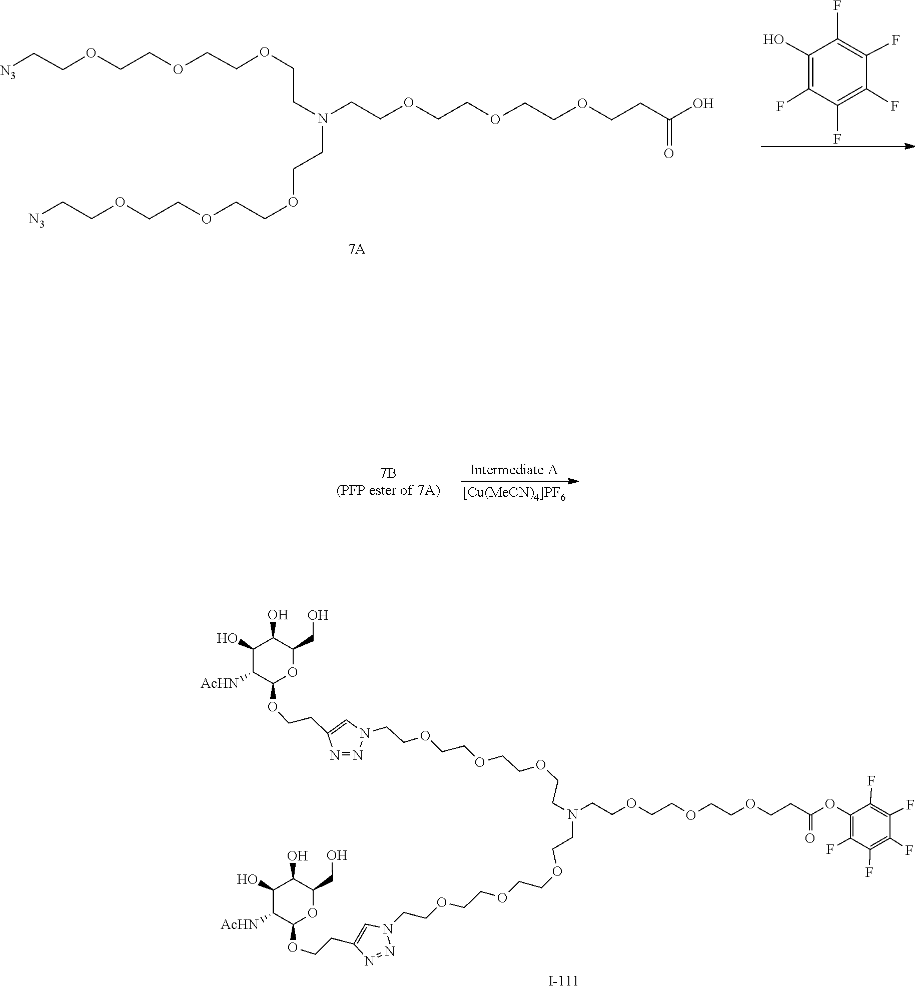 [SMILES: [N-]=[N+]=NCCOCCOCCOCCN(CCOCCOCCOCCN=[N+]=[N-])CCOCCOCCOCCC(=O)O ; Oc1c(F)c(F)c(F)c(F)c1F ; CC(=O)N[C@H]1[C@H](OCCc2cn(CCOCCOCCOCCN(CCOCCOCCOCCC(=O)Oc3c(F)c(F)c(F)c(F)c3F)CCOCCOCCOCCn3cc(CCO[C@@H]4O[C@H](CO)[C@H](O)[C@H](O)[C@H]4NC(C)=O)nn3)nn2)O[C@H](CO)[C@H](O)[C@@H]1O]

N-(acid-PEG3)-N-bis(PEG3-azide) (7A) (1.00 eq) and DIPC (1.00 eq) are dissolved with stirring in NMP. After 5 min a solution of 2,3,4,5,6-pentafluorophenol (1.50 eq) in NMP is added. The resulting clear solution is capped and stirred at room temperature for 2 h at which time a catalytic amount of DMAP is added. After 24 h the resulting mixture is added to Intermediate A (2.00 eq.) in a 1 dram vial with a stirbar. After 2 min, tetrakis(acetonitrile)copper(I) hexafluorophosphate (5.00 eq, 54.7 mg, 0.147 mmol) is added. The resulting light yellow solution is capped and stirred at room temperature for 30 min. The reaction mixture is diluted with a mixture of NMP, ethanol, and acetic acid, filtered, and purified via preparatory HPLC. Fractions containing the desired product are combined and lyophilized to dryness to afford Compound I-111.

### ASGPR Example 112

### Synthesis of GalNac ligand A Periluorophenyl Ester (Compound I-112)

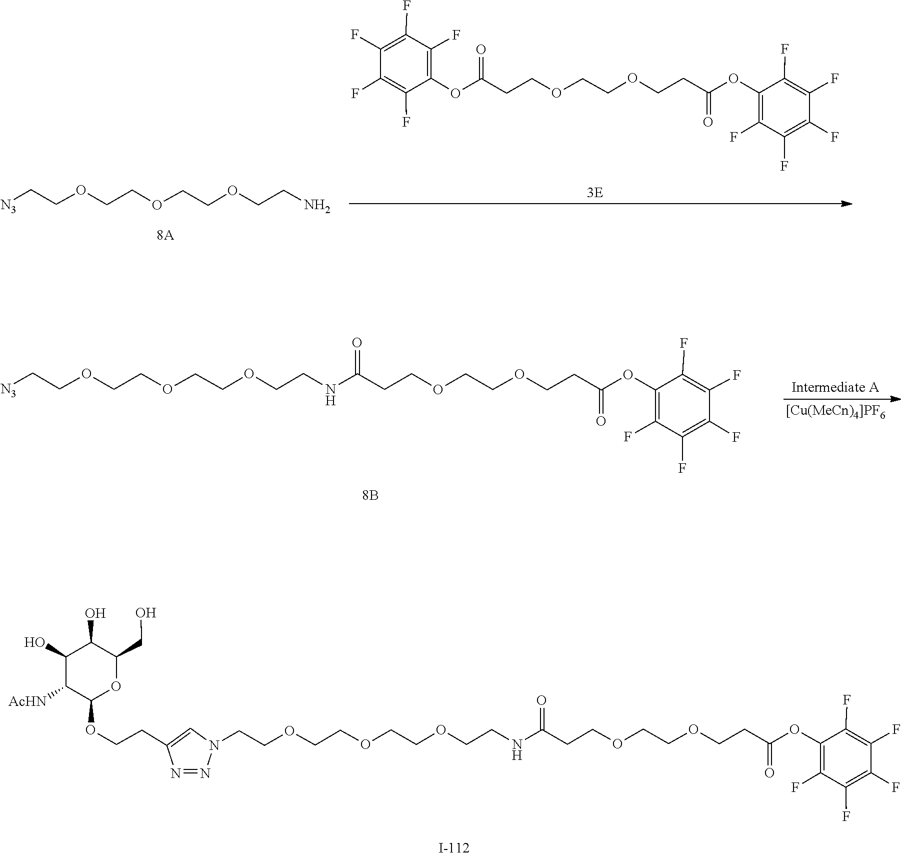 [SMILES: O=C(CCOCCOCCC(=O)Oc1c(F)c(F)c(F)c(F)c1F)Oc1c(F)c(F)c(F)c(F)c1F ; [N-]=[N+]=NCCOCCOCCOCCN ; [N-]=[N+]=NCCOCCOCCOCCNC(=O)CCOCCOCCC(=O)Oc1c(F)c(F)c(F)c(F)c1F ; CC(=O)N[C@H]1[C@H](OCCc2cn(CCOCCOCCOCCNC(=O)CCOCCOCCC(=O)Oc3c(F)c(F)c(F)c(F)c3F)nn2)O[C@H](CO)[C@H](O)[C@@H]1O]

A solution of bis(perfluorophenyl) 3,3′-(ethane-1,2-diylbis(oxy))dipropionate (Compound 3E) (1 eq.) in NMP is added to a solution of 2-(2-(2-(2-azidoethoxy)ethoxy)ethoxy)ethan-1-amine (8A) (1 eq.) in NMP. The resulting mixture is stirred at room temperature for 30 min and then added to Intermediate A (1 eq.). After stirring for 5 min tetrakis(acetonitrile)copper(I) hexafluorophosphate (3 eq) is added. The resulting mixture is stirred at room temperature for 30 min. The reaction mixture is diluted with a mixture of NMP, ethanol, and acetic acid, filtered, and purified via preparatory HPLC. Fractions containing the desired product are combined and lyophilized to dryness to afford Compound I-8.

Compound I-8 was synthesized in the following alternative steps.

To a solution of Compound 3E (1.0 eq, 0.50 g, 0.929 mmol) in tetrahydrofuran (5 mL, 10 vol.) was added Compound 8A (1.0 eq, 0.203 g, 0.929 mmol) and N,N-diisopropylethylamine (2.0 eq, 0.34 mL, 1.86 mmol). The reaction mixture was allowed to stir at room temperature for 2 h. The progress of reaction was monitored by LCMS. When complete, the reaction mixture was diluted with acetonitrile and purified by reverse-phase prep H PLC (55-65% acetonitrile in water with 0.1% TFA). Fractions containing the desired product were combined and lyophilized to dryness to afford perfluorophenyl 1-azido-13-oxo-3,6,9,16,19-pentaoxa-12-azadocosan-22-oate (Compound 8B) as a colorless viscous liquid. Yield: 0.130 g, 23%; LCMS m/z 573.25 [M+1]+.

To a solution of Compound 8B (1.0 eq, 0.070 g, 0.122 mmol) in dimethyl sulfoxide (2 mL) was added Intermediate A (1.0 eq, 0.0334 g, 0.122 mmol). The reaction mixture was stirred for 5 minutes prior to addition of tetrakis(acetonitrile)copper(I) hexafluorophosphate (2.5 eq, 0.100 g, 0.306 mmol). The reaction mixture was stirred at room temperature for 1 h. The progress of reaction was monitored by LCMS. After completion, reaction mixture was diluted with acetonitrile and purified by prep HPLC (35-55% acetonitrile in water with 0.1% TFA). Fractions containing the desired product were combined and lyophilized to dryness to afford the Compound I-112 as a colorless viscous liquid. Yield: 0.015 g, 14.5%; LCMS m/z 846.33 [M+1]+; 1H NMR (400 MHz, D20) 7.83 (s, 1H), 4.60-4.58 (m, 2H), 4.43 (d, J=8.4 Hz, 1H), 4.17-4.13 (m, 1H), 3.97-3.90 (m, 5H), 3.88-3.72 (m, 6H), 3.70-3.49 (m, 16H), 3.37-3.34 (m, 2H), 3.05 (t, J=6.0 Hz, 2H), 2.96 (t, J=6.0 Hz, 2H), 2.50 (t, J=6.0 Hz, 2H), 1.84 (s, 3H).

### ASGPR Example 113

### Synthesis of perfluorophenyl 1-(4-(2-(((2R,3R,4R,5R,6R)-5-acetamido-3,4,6-trihydroxytetrahydro-2H-pyran-2-yl)methoxy)ethyl)-1H-1,2,3-triazol-1-yl)-13-oxo-3,6,9,16,19-pentaoxa-12-azadocosan-22-oate (Compound I-113)

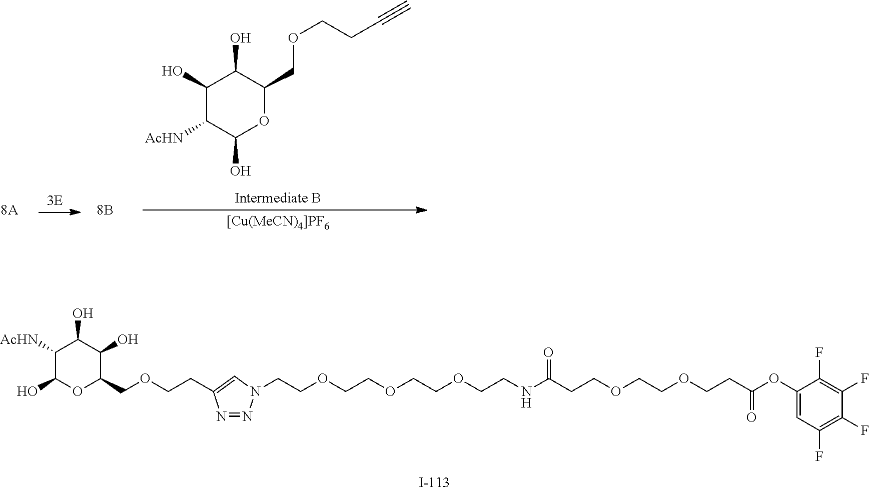 [SMILES: C#CCCOC[C@H]1O[C@@H](O)[C@H](NC(C)=O)[C@@H](O)[C@H]1O ; CC(=O)N[C@@H]1[C@@H](O)[C@@H](O)[C@@H](COCCc2cn(CCOCCOCCOCCNC(=O)CCOCCOCCC(=O)Oc3cc(F)c(F)c(F)c3F)nn2)O[C@H]1O]

A solution of bis(perfluorophenyl) 3,3′-(ethane-1,2-diylbis(oxy))dipropionate (Compound 3E) (1 eq.) in NMP is added to a solution of 2-(2-(2-(2-azidoethoxy)ethoxy)ethoxy)ethan-1-amine (8A) (1 eq.) in NMP. The resulting mixture is stirred at room temperature for 30 min and then added to Intermediate B (1 eq.). After stirring for 5 min tetrakis(acetonitrile)copper(I) hexafluorophosphate (3 eq) is added. The resulting mixture is stirred at room temperature for 30 min. The reaction mixture is diluted with a mixture of NMP, ethanol, and acetic acid, filtered, and purified via preparatory HPLC. Fractions containing the desired product are combined and lyophilized to dryness to afford Compound I-113.

### ASGPR Example 114: Synthesis of Compound I-114

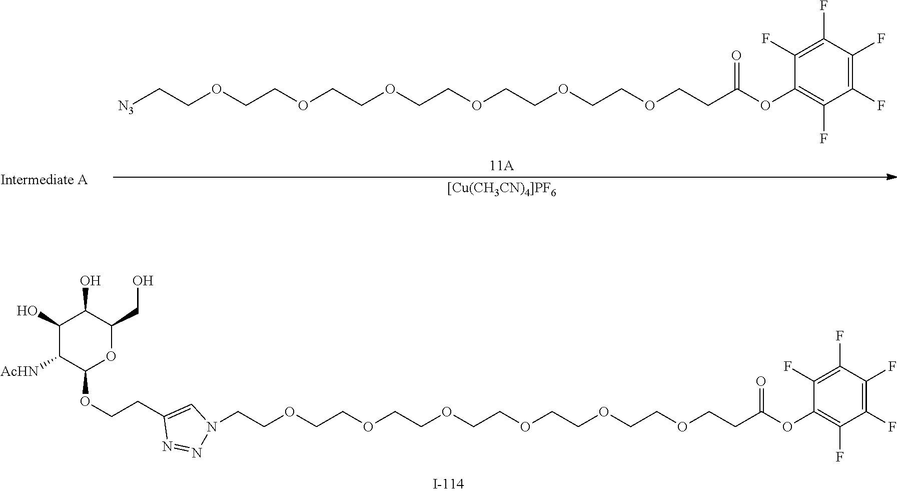

To a solution of perfluorophenyl 1-azido-3,6,9,12,15,18-hexaoxahenicosan-21-oate (10A) (1.0 eq, 0.0998 g, 0.183 mmol) in dimethyl sulfoxide (1 mL), Intermediate A (1.0 eq, 0.050 g, 0.183 mmol) was added and stirred for 5 minutes. Then, tetrakis(acetonitrile)copper(I) hexafluorophosphate (2.5 eq, 0.170 g, 0.457 mmol) was added and reaction mixture was stirred at room temperature for 1 h. The progress of reaction was monitored by LC-MS. After completion, reaction mixture was diluted with acetonitrile and purified by prep H PLC (30-45% acetonitrile in water with 0.1% acetic acid). Fractions containing the desired product were combined and lyophilized to dryness to afford Compound I-114 as an off white solid. Yield: 0.022 g, 14.1%; LC-MS m/z 819.24 [M+1]⁺; ¹H NMR (400 MHz, D₂O) δ 7.81 (s, 1H), 4.57-4.55 (m, 2H), 4.40 (d, J=19.2 Hz, 1H), 4.16-4.11 (m, 1H), 3.94-3.85 (m, 6H), 3.80-3.73 (m, 3H), 3.71-3.59 (m, 22H), 3.04 (t, J=5.6 Hz, 2H), 2.94 (t, J=6.0 Hz, 2H), 1.81 (s, 3H).

### ASGPR Example 115: Synthesis of Compound I-115

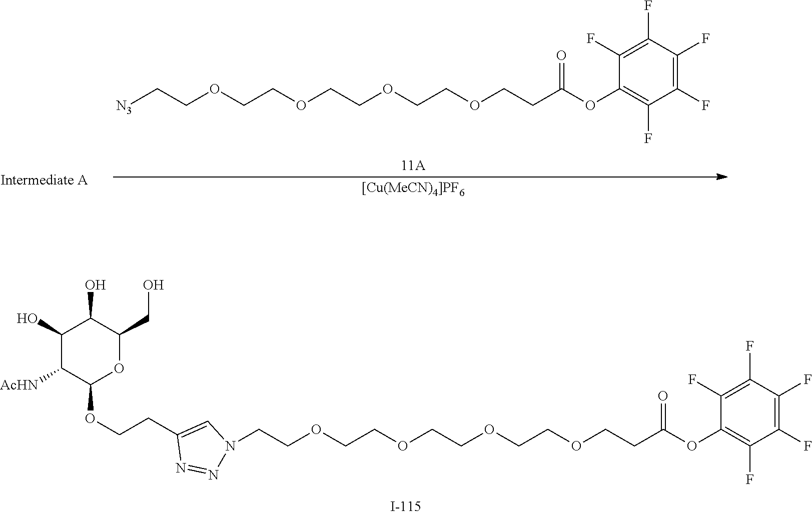 [SMILES: [N-]=[N+]=NCCOCCOCCOCCOCCC(=O)Oc1c(F)c(F)c(F)c(F)c1F ; CC(=O)N[C@H]1[C@H](OCCc2cn(CCOCCOCCOCCOCCC(=O)Oc3c(F)c(F)c(F)c(F)c3F)nn2)O[C@H](CO)[C@H](O)[C@@H]1O]

To a solution of 2,3,4,5,6-pentafluorophenyl 1-azido-3,6,9,12-tetraoxahexadecan-16-oate (11A) (86.2 mg, 183 μmol) in dimethylsulfoxide (2 mL) was added Intermediate A (50.0 mg, 183 μmol) and stirred for 5 minutes, then tetrakis(acetonitrile)copper(I) hexafluorophosphate (0.168 g, 0.512 mmol) was added and reaction mixture was stirred at room temperature for 1 h. After completion, reaction mixture was diluted with acetonitrile and purified by prep HPLC (35-55% acetonitrile in water with 0.1% TFA). Fractions containing the desired product were combined and lyophilized to dryness to afford Compound I-115 as a colorless viscous liquid. Yield: 0.015 g, 11%; LC-MS m/z 731.2 [M+1]⁺; ¹H NMR (400 MHz, D₂O) δ 7.82 (2, 1H), 4.60-4.57 (m, 2H), 4.43 (d, J=8.1 Hz, 1H), 4.20-4.12 (m, 1H), 3.96-3.90 (m, 4H), 3.85-3.57 (m, 17H), 3.25-3.15 (m, 1H), 3.08-3.04 (m, 2H), 2.98-2.94 (m, 2H), 1.83 (s, 3H), 1.27 (t, J=7.16, 2H).

### ASGPR Example 116: Synthesis of Compound I-116

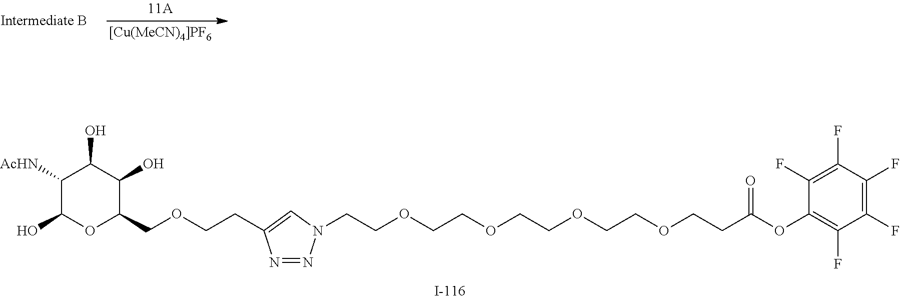 [SMILES: CC(=O)N[C@@H]1[C@@H](O)[C@@H](O)[C@@H](COCCc2cn(CCOCCOCCOCCOCCC(=O)Oc3c(F)c(F)c(F)c(F)c3F)nn2)O[C@H]1O]

Compound 11A (1 eq.) and Intermediate B (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-116, which is purified via reverse-phase preparatory HPLC followed by lyophilization.

### ASGPR Example 117: Synthesis of Compound I-117

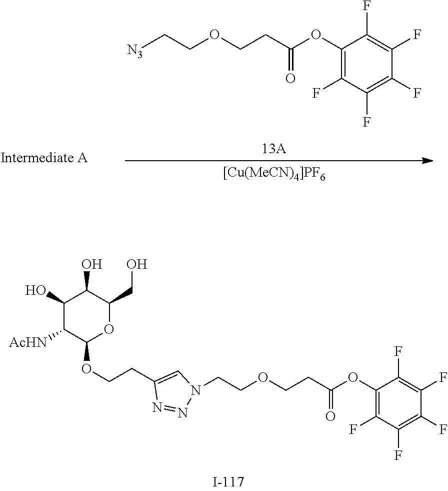 [SMILES: [N-]=[N+]=NCCOCCC(=O)Oc1c(F)c(F)c(F)c(F)c1F ; CC(=O)N[C@H]1[C@H](OCCc2cn(CCOCCC(=O)Oc3c(F)c(F)c(F)c(F)c3F)nn2)O[C@H](CO)[C@H](O)[C@@H]1O]

Compound 13A (1 eq.) and Intermediate A (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-113, which is purified via reverse-phase preparatory HPLC followed by lyophilization.

Compound I-117 was synthesized in the following alternative steps. To a solution of perfluorophenyl 3-(2-azidoethoxy)propanoate (Compound 13A) (1.0 eq, 0.07 g, 0.220 mmol) in dimethylsulfoxide (2 mL), Intermediate A (1.0 eq, 0.06 g, 0.220 mmol) was added and stirred for 5 minutes. Then, tetrakis(acetonitrile)copper(I) hexafluorophosphate (2.5 eq, 0.204 g, 0.549 mmol) was added and reaction mixture was stirred at room temperature for 1 h. The progress of reaction was monitored by LCMS. After completion, reaction mixture was diluted with acetonitrile and purified by prep HPLC (30-52% acetonitrile in water with 0.1% acetic acid). Fractions containing the desired product were combined and lyophilized to dryness to afford Compound I-117 as a white solid. Yield: 0.020 g, 15%; LC-MS m/z 599.1 [M+1]+; 1H NMR (400 MHz, D20) d 7.75 (s, 1H), 4.78-4.57 (m, 2H), 4.39 (d, J=8.1 Hz, 1H), 4.15-4.08 (m, 2H), 3.99 (t, J=4.6 Hz, 2H), 3.90-3.86 (m, 3H), 3.81-3.72 (m, 4H), 3.67-3.64 (m, 2H), 2.97 (t, J=5.6 Hz, 2H), 2.86 (t, J=6.4 Hz, 2H), 1.83 (s, 3H).

### ASGPR Example 118: Synthesis of Compound I-118

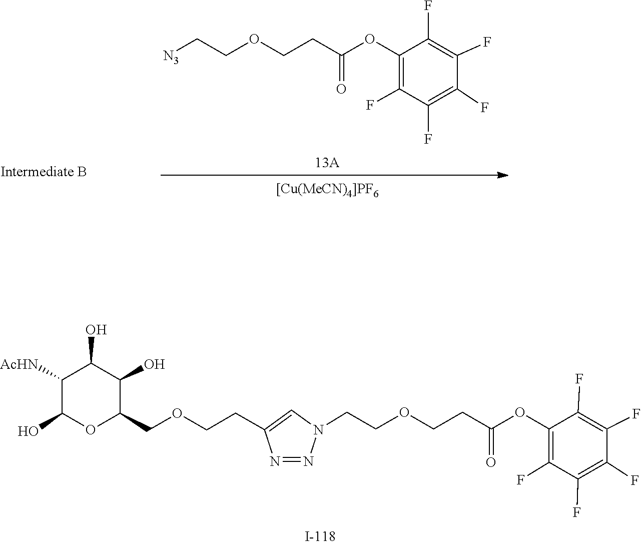 [SMILES: [N-]=[N+]=NCCOCCC(=O)Oc1c(F)c(F)c(F)c(F)c1F ; CC(=O)N[C@@H]1[C@@H](O)[C@@H](O)[C@@H](COCCc2cn(CCOCCC(=O)Oc3c(F)c(F)c(F)c(F)c3F)nn2)O[C@H]1O]

Compound 13A (1 eq.) and Intermediate B (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-118, which is purified via reverse-phase preparatory HPLC followed by lyophilization.

### ASGPR Example 119: Synthesis of Intermediate C

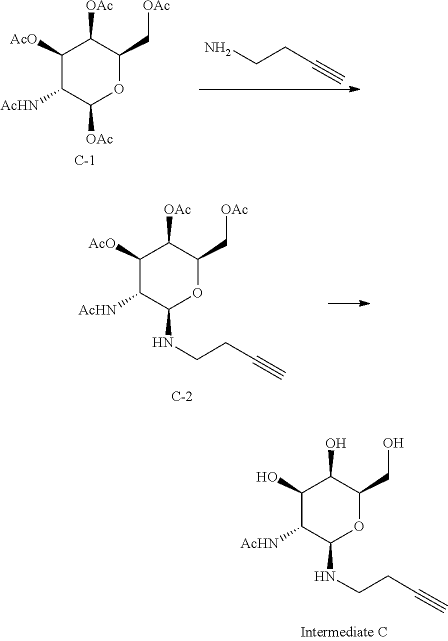

To activated 4 Å molecular sieves and [(2R,3R,4R,5R,6S)-3,4,6-tris(acetyloxy)-5-acetamidooxan-2-yl]methyl acetate (C-1) (1 eq.) is added dichloromethane. To the reaction solution is added but-3-yn-1-amine (3 eq). The reaction mixture is allowed to cool to 0° C. prior to the addition of diethyl trifluoroborinate (2 eq). The reaction is stirred at room temperature and then heated to refluxing for 16h. Aqueous NaHCO₃ is added to quench the diethyl trifluoroborinate and the DCM layer is partitioned and dried over MgSO₄. The solution is filtered and concentrated on a rotary evaporator. Silica gel column purification with 60-75% ethyl acetate in dichloromethane as eluent is used to obtain Intermediate C-2.

Intermediate C-2 (1 eq.) is dissolved in methanol and cooled to 0° C. Sodium methoxide 25% w/v (10 eq) in methanol is added dropwise to this solution. The reaction is maintained at room temperature for 3 h. After completion of reaction 1N HCl is added dropwise to quench the sodium methoxide. Methanol is evaporated and the obtained residue is washed with diethyl ether. The crude residue obtained is purified with prep-HPLC (5-20% acetonitrile in water with 0.1% TFAH) to afford Intermediate C.

### ASGPR Example 120: Synthesis of Compound I-120

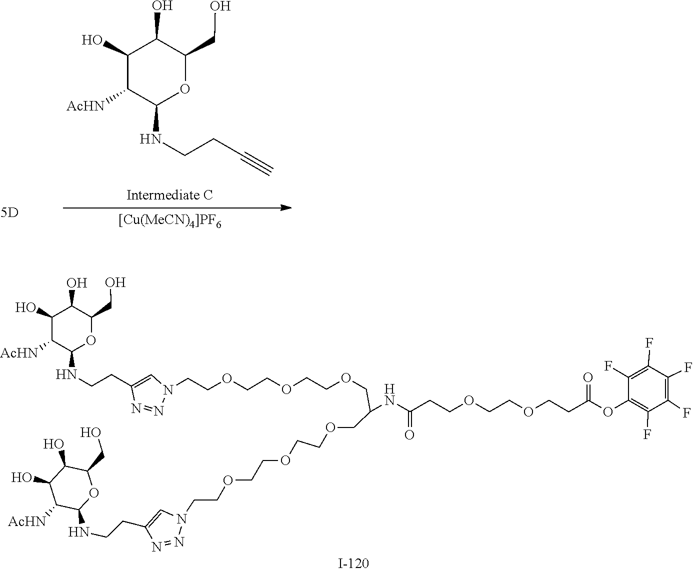 [SMILES: C#CCCN[C@@H]1O[C@H](CO)[C@H](O)[C@H](O)[C@H]1NC(C)=O ; CC(=O)N[C@@H]1[C@@H](O)[C@@H](O)[C@@H](CO)O[C@H]1NCCc1cn(CCOCCOCCOCC(COCCOCCOCCn2cc(CCN[C@@H]3O[C@H](CO)[C@H](O)[C@H](O)[C@H]3NC(C)=O)nn2)NC(=O)CCOCCOCCC(=O)Oc2c(F)c(F)c(F)c(F)c2F)nn1]

Compound 5D (1 eq.) and Intermediate C (1 eq.) are dissolved with stirring in DMSO at room temperature. Tetrakis(acetonitrile)copper(I) tetrafluoroborate (3 eq.) is added and the resulting mixture is stirred at room temperature until LC-MS indicates complete conversion to Compound I-120, which is purified via reverse-phase preparatory HPLC followed by lyophilization.

### ASGPR Example 121: Synthesis of Compound I-121

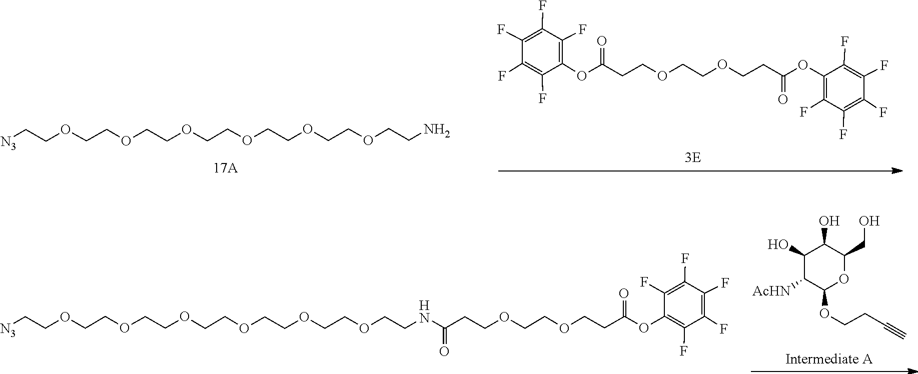 [SMILES: [N-]=[N+]=NCCOCCOCCOCCOCCOCCOCCN ; O=C(CCOCCOCCC(=O)Oc1c(F)c(F)c(F)c(F)c1F)Oc1c(F)c(F)c(F)c(F)c1F ; [N-]=[N+]=NCCOCCOCCOCCOCCOCCOCCNC(=O)CCOCCOCCC(=O)Oc1c(F)c(F)c(F)c(F)c1F ; C#CCCO[C@@H]1O[C@H](CO)[C@H](O)[C@H](O)[C@H]1NC(C)=O]

Compound 17B was synthesized employing the procedures described for Compound 8B using Compound 17A in lieu of Compound 8A. Compound I-121 was synthesized employing the procedures described for Compound I-8 using Compound 17B in lieu of Compound 8B (32 mg). LC-MS m/z 978.3 [M+1]⁺.

### ASGPR Example 122: Synthesis of Compound I-122

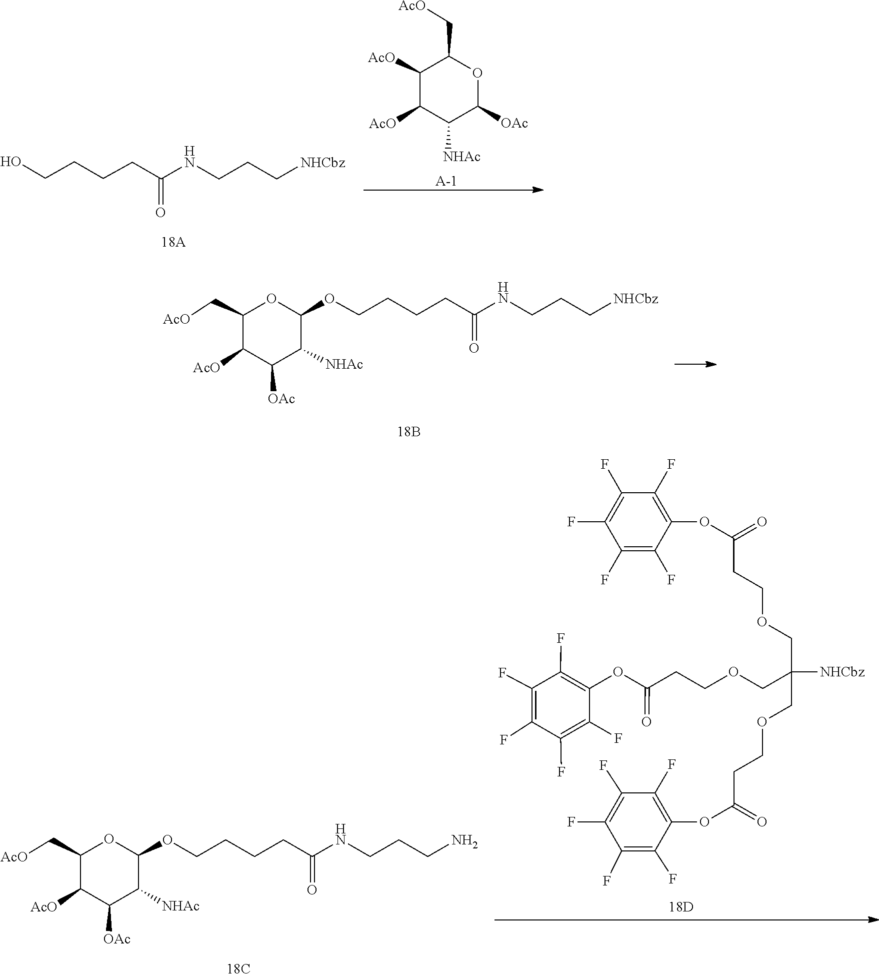

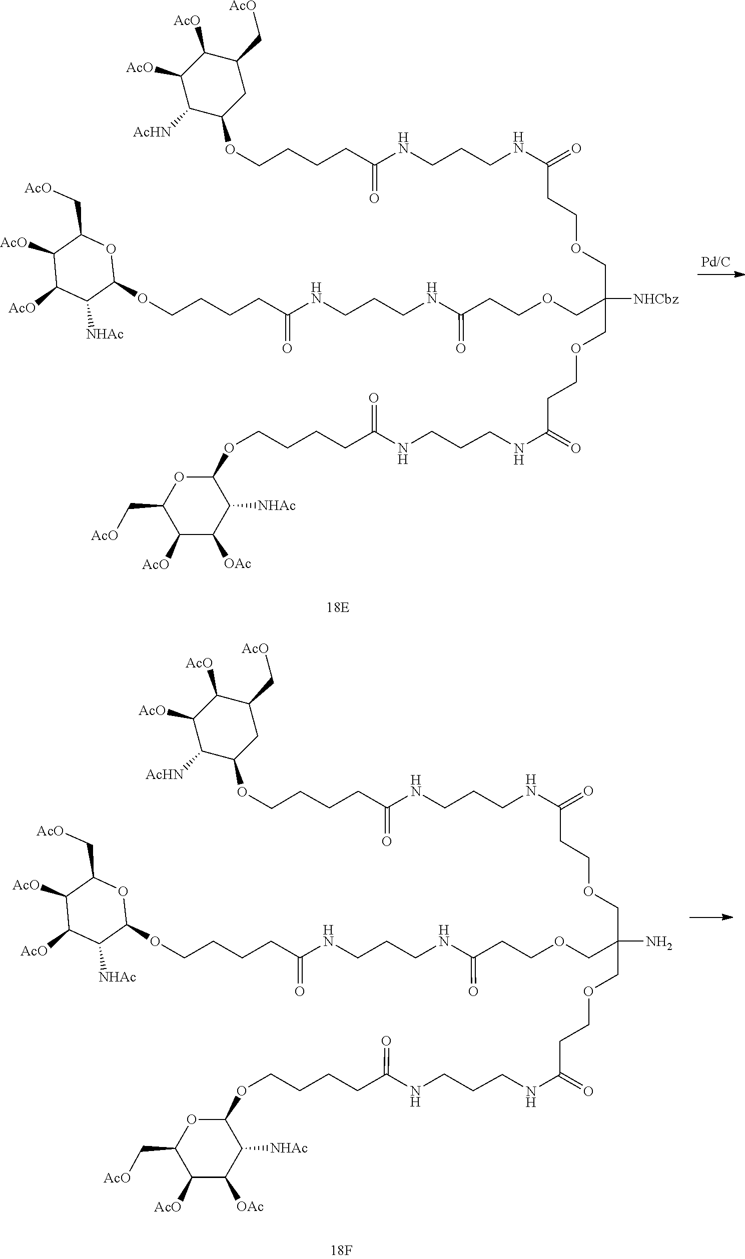

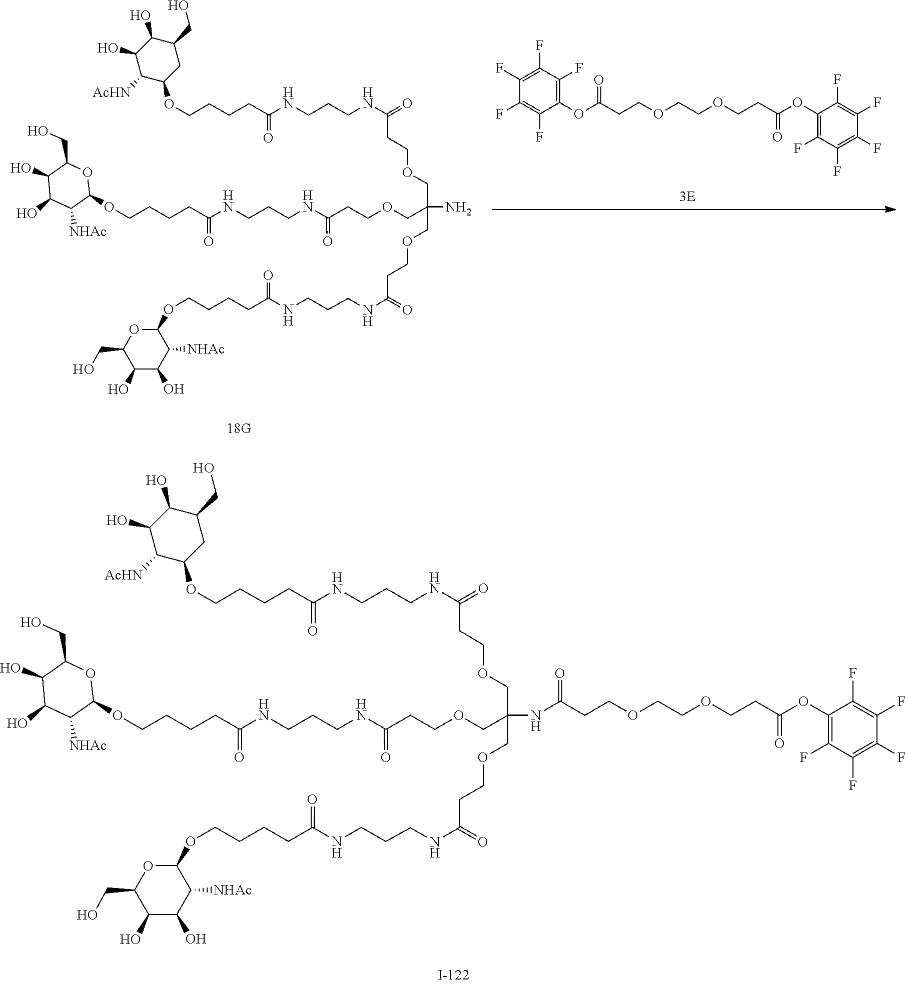

To the solution of Compound A-1 (1.0 eq, 5.05 g, 13.0 mmol) and benzyl N-[3-(5-hydroxypentanamido) prop yl]carbamate (Compound 18A) (1.0 eq, 4.00 g, 13.0 mmol) in dichloromethane (50.0 mL), trimethylsilyl trifluoromethanesulfonate (1.1 eq, 2.52 mL, 14.3 mmol) was added dropwise at room temperature. The reaction mixture was stirred at 40° C. for 5 h. After completion, the reaction mixture was quenched with saturated sodium bicarbonate solution and extracted with dichloromethane. The organic layer was dried over sodium sulfate, filtered, and concentrated under high vacuum to get crude. The crude was purified by reverse phase chromatography using 0-30% acetonitrile in water to afford Compound 18B as yellow viscous liquid, Yield: (5.80 g, 70.12%); LCMS m/z 638.2 [M+1]⁺

To a solution of Compound 18B (1.0 eq, 4.80 g, 7.53 mmol) in methanol (40.0 mL), 10% Palladium on carbon (1.60 g) was added and stirred at room temperature under hydrogen atmosphere for 4 h. After completion, the reaction mixture was filtered through syringe filter, filtrate was concentrated and dried to get crude. The crude was triturated with diethyl ether to afford Compound 18C as a pale yellow viscous liquid. Yield: (3.4 g, 80.73%); LCMS m/z 504.37 [M+1]⁺.

A solution of 2,3,4,5,6-pentafluorophenyl 3-(2-{[(benzyloxy)carbonyl]amino}-3-[3-oxo-3-(2,3,4,5,6-pentafluorophenoxy)propoxy]-2-{[3-oxo-3-(2,3,4,5,6-pentafluorophenoxy)propoxy]methyl}propoxy)propanoate (18D) (1.0 eq, 1.20 g, 1.24 mmol) and Compound 18C (3.0 eq, 1.87 g, 3.71 mmol) in N,N-dimethylformamide (30.0 mL) was stirred at room temperature for 1 h. After completion, the reaction mixture was concentrated and dried to get crude. The crude was purified by flash column chromatography using 20% methanol in dichloromethane to afford Compound 18E as pale yellow viscous liquid. Yield: (1.60 g; 67.05%); LCMS m/z 1926.78 [M−1]⁻.

To a solution of Compound 18E (1.0 eq, 1.60 g, 0.830 mmol) in methanol (20 mL) and acetic acid (1.0 mL), 10% Palladium on carbon (250 mg) was added. The reaction mixture was stirred at room temperature under hydrogen atmosphere for 16 h. After completion, the reaction mixture was filtered through celite bed, filtrate was concentrated and dried to afford Compound 18F as pale yellow viscous liquid. Yield: 1.45 g (Crude); LCMS m/z 1794.05 [M+1]⁺.

To a solution of Compound 18F (1.0 eq, 1.45 g, 0.808 mmol) in methanol (10 mL), 25% sodium methanolate solution (8.0 eq, 1.45 mL, 6.47 mmol) was added at 0° C. The reaction mixture was stirred at room temperature for 1h. After completion reaction, reaction mixture was concentrated and dry to get crude. The crude was diluted with acetonitrile and purified by prep HPLC (30% acetonitrile in water with 0.1% TFA). Fractions containing the desired product were combined and lyophilized to dryness to afford Compound 18G as an off white semi solid. Yield: (0.20 g, 17.4%); LCMS m/z 1415.77 [M+1]⁺.

To a solution of Compound 18G (1.0 eq, 0.090 g, 0.0636 mmol) in dimethyl sulfoxide (1.00 mL), Compound 3E (1.0 eq, 0.030 g, 0.0636 mmol) was added and stirred at room temperature for 16 h. After completion, reaction mixture was diluted with acetonitrile and purified by prep HPLC (42 acetonitrile in water with 0.1% Acetic acid (0-13 min)). Fractions containing the desired product were combined and lyophilized to dryness to afford Compound I-122 as off white solid. Yield: 0.004 g, 3.55%; LC-MS m/z 1769.93 [M+1]⁺; ¹H NMR (400 MHz, DMSO-d₆) δ 7.84 (bs, 3H), 7.73 (bs, 3H), 7.63 (d, J=9.2 Hz, 3H), 7.13 (s, 1H), 4.58-4.54 (m, 4H), 4.47 (bs, 3H), 4.22 (d, J=8.8 Hz, 3H), 3.77-3.67 (m, 12H), 3.53-3.52 (m, 30H), 3.32-3.27 (m, 4H), 3.02 (bs, 14H), 2.29 (t, J=6.0 Hz, 6H), 2.05 (t, J=7.2 Hz, 6H), 1.79 (s, 9H), 1.50-1.41 (m, 18H).

### ASGPR Example 123: Synthesis of N-[1,3-bis(2-{[3-(5-{[(2R,3R,4R,5R,6R)-3-acetamido-4,5-dihydroxy-6-(hydroxymethyl)oxan yl]oxy}pentanamido)propyl]carbamoyl}ethoxy)-2-[(2-{[3-(5-{[(2R,3R,4R,5R,6R) acetamido-4,5-dihydroxy-6-(hydroxymethyl)oxan yl]oxy}pentanamido)propyl]carbamoyl}ethoxy)methyl]propan-2-yl]-12-(2,5-dioxo-2,5-dihydro-1H-pyrrol-1-yl)dodecanamide (Compound I-123)

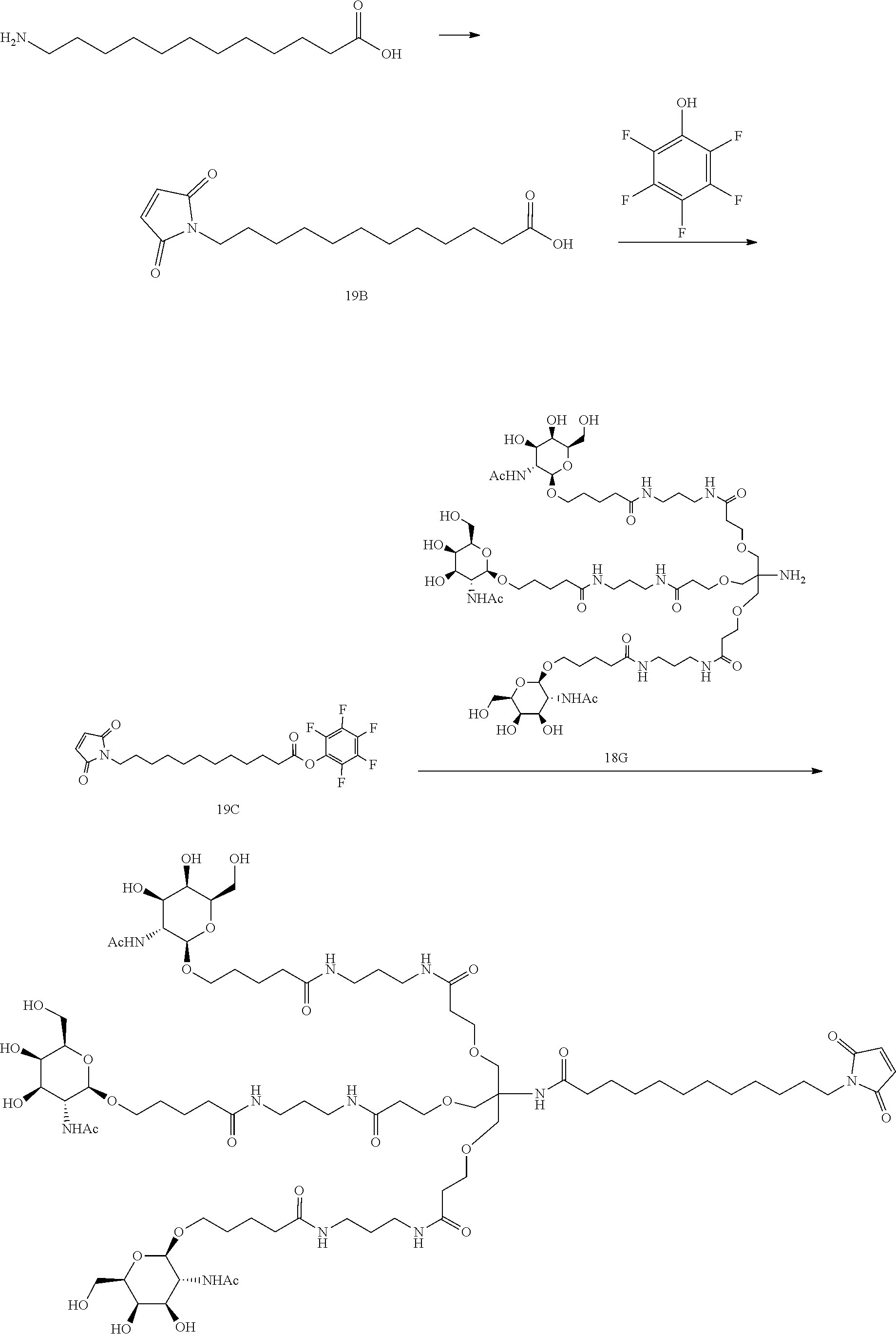

To a solution of 12-aminododecanoic acid (19A) (2.00 g, 9.29 mmol) in acetic acid (15.00 ml) was added 2,5-dihydrofuran-2,5-dione (1.09 g, 11.1 mmol) and reaction mixture was refluxed at 120° C. for 16 h. After completion, reaction mixture was concentrated under vacuum to get crude compound which was purified by flash column chromatography using silica gel and 5% methanol in dichloromethane as eluents to afford Compound 19B as off white solid. Yield:1.60 g (57.17%); LCMS m/z 294.3 [M−1]⁻.

To a solution of Compound 19B (0.300 g, 1.02 mmol) in tetrahydrofuran (15.00 mL) at 0° C. were added pentafluorophenol (168 mg, 0.914 mmol) and diisopropylmethanediimine (0.192 mL, 1.22 mmol). Reaction mixture was then stirred at room temperature for 1 h. After completion reaction mixture was concentrated to get crude product which was purified by flash column chromatography using silica gel and 5% to 7% ethyl acetate in hexanes as eluents to afford Compound 19C as off white solid. Yield: 0.250 g (53.34%) ELSD-MS m/z 479.0[M+18]⁺.

Compound 18G (0.060 g, 0.04 mmol) in dimethylsulfoxide (1.0 mL), N,N-diisopropylethylamine (0.015 mL, 0.084 mmol) and Compound 19C (0.019 g, 0.04 mmol) were added and reaction mixture was stirred at room temperature for 16 h. After completion, reaction mixture was diluted with acetonitrile and purified by prep HPLC (25-45% acetonitrile in water with 0.1% TFA). Fractions containing the desired product were combined and lyophilized to dryness to afford Compound I-123 as an off-white solid. Yield: 0.0035 g, 4.88%; LC-MS m/z 1692.93[M+1]⁺; ¹H NMR (400 MHz, DMSO-d₆) δ 7.83 (t, J=5.2 Hz, 3H), 7.73 (t, J=6.0 Hz, 3H), 7.61 (d, J=9.6 Hz, 3H), 6.98 (s, 3H), 4.57-4.53 (m, 7H), 4.22 (d, J=8.4 Hz, 3H), 3.72-3.63 (m, 9H), 3.55-3.51 (m, 20H), 3.37-3.27 (m, 10H), 3.04-3.01 (m, 12H), 2.29 (t, J=6.4 Hz, 6H), 2.05 (t, J=6.8 Hz, 6H), 1.81 (bs, 8H), 1.51-1.41 (m, 24H), 1.21 (s, 14H).

### ASGPR Example 124: Synthesis of Compound I-124

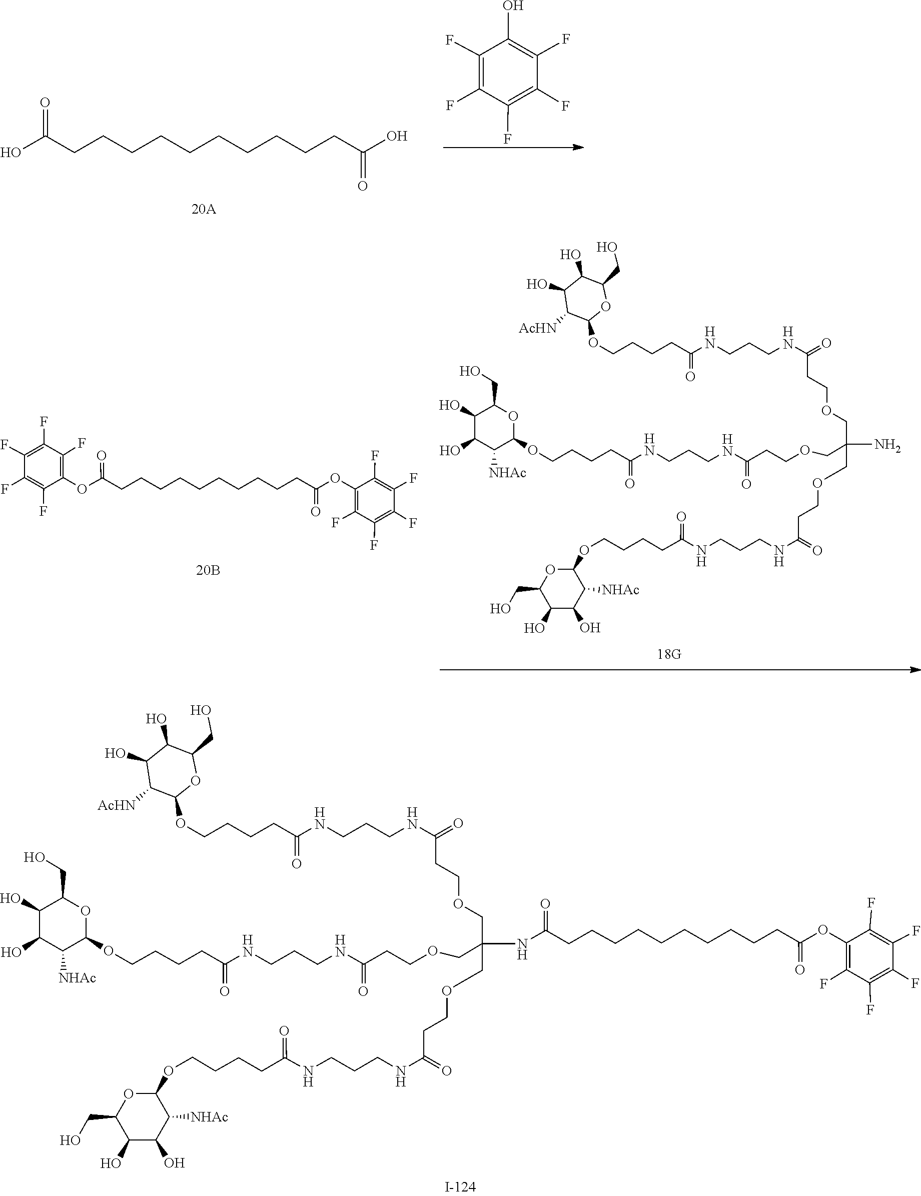

To the solution of dodecanedioic acid (20A) (1.00 g, 4.34 mmol) in ethyl acetate (10.00 mL) at 0° C., pentafluorophenol (1.60 g, 8.68 mmol) and diisopropylmethanediimine (1.91 mL, 13.0 mmol) were added and reaction mixture stirred at room temperature for 1h. After completion, reaction mixture was filtered through celite bed and filtrate was concentrated under reduced pressure to get crude compound. Crude compound obtained was purified by flash column chromatography on silica gel column using 5% ethyl acetate in hexanes as eluents to afford Compound 20B as off white solid. Yield: 1.00 g (40.95%); LCMS m/z 580.39 [M+18]⁺.

To a solution Compound 18G (45.0 mg, 0.031 mmol) in dimethyl sulfoxide (1.0 mL) was added N,N-diisopropylethylamine (0.016 mL, 0.093 mmol) and Compound 20B (17.9 mg, 0.031 mmol). Reaction mixture was stirred at room temperature for 2 h. After completion, the reaction mixture was purified via preparatory HPLC (40-60% acetonitrile in water with 0.1% trifluoroacetic acid). Fractions containing the desired product were combined and lyophilized to dryness to afford Compound I-124 as an off white solid. Yield: 0.006 g (10.52%); LCMS m/z 1793.94 [M+1]⁺, 897.99 [M/2+1]⁺. ¹H NMR (400 MHz, DMSO-d6) δ 7.83 (t, J=5.6 Hz, 3H), 7.73 (t, J=5.2 Hz, 3H), 7.60 (d, J=9.2 Hz, 3H), 6.99 (s, 1H), 4.57-4.47 (m, 6H), 4.46 (d, J=4.4 Hz, 3H), 4.21 (d, J=8.4 Hz, 3H), 3.70-3.63 (m, 9H), 3.55-3.49 (m, 21H), 3.32-3.28 (m, 4H), 3.02 (t, J=5.6 Hz, 12H), 2.76 (t, J=5.6 Hz, 2H), 2.27 (t, J=6.4 Hz, 6H), 2.03 (t, J=7.2 Hz, 8H), 1.79 (s, 9H), 1.70-1.67 (m, 2H), 1.52-1.41 (m, 20H), 1.23 (bs, 14H).

### ASGPR Example 125: Synthesis of Compound I-125

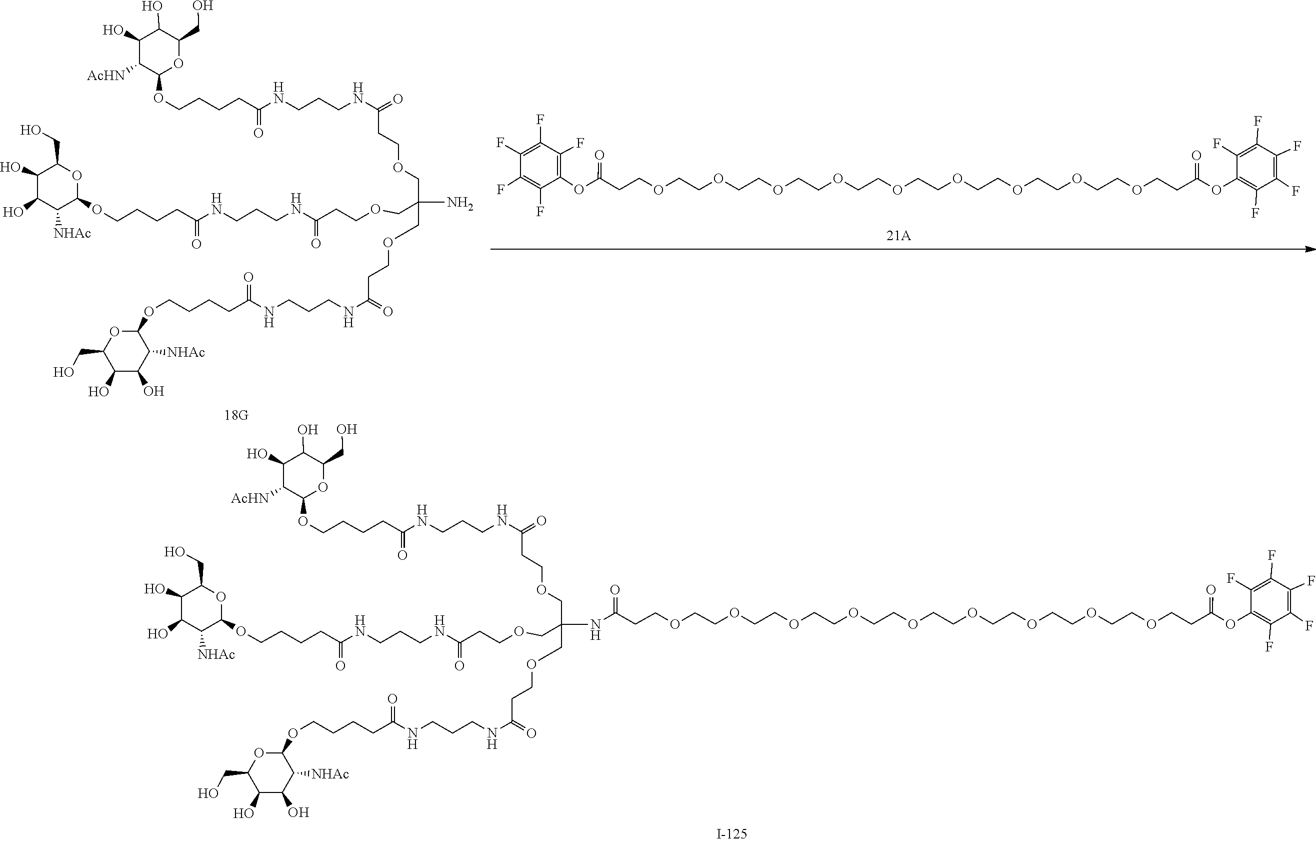

To a solution of Compound 18G (1.0 eq, 0.10 g, 0.070 mmol) in dimethyl sulfoxide (1.00 mL), ethylbis(propan-2-yl)amine (3.0 eq, 39.1 μL, 0.212 mmol) and bis(2,3,4,5,6-pentafluorophenyl) 4,7,10,13,16,19,22,25,28-nonaoxahentriacontanedioate (21A) (1.0 eq, 0.0598 g, 0.070 mmol) were added and stirred at room temperature for 16 h. After completion, reaction mixture was diluted with acetonitrile and purified by prep HPLC (50% acetonitrile in water with 0.1% Acetic acid (0-10 min)). Fractions containing the desired product were combined and lyophilized to dryness to afford Compound I-125 as off white solid. Yield: 0.006 g, 4.09%; LC-MS m/z 1039.74 [M/2+1]⁺; ¹HNMR (400 MHz, D₂O) δ 4.45 (d, J=8.4 Hz, 3H), 3.96-3.83 (m, 11H), 3.80-3.58 (m, 61H), 3.24-3.19 (m, 12H), 3.10 (t, J=5.6 Hz, 2H), 2.52-2.47 (m, 8H), 2.27 (t, J=6.0 Hz, 6H), 2.02 (s, 9H), 1.75-1.70 (m, 6H), 1.58-1.50 (m, 12H), 1.35-1.34 (m, 1H).

### ASGPR Example 126: Synthesis of Compound I-126

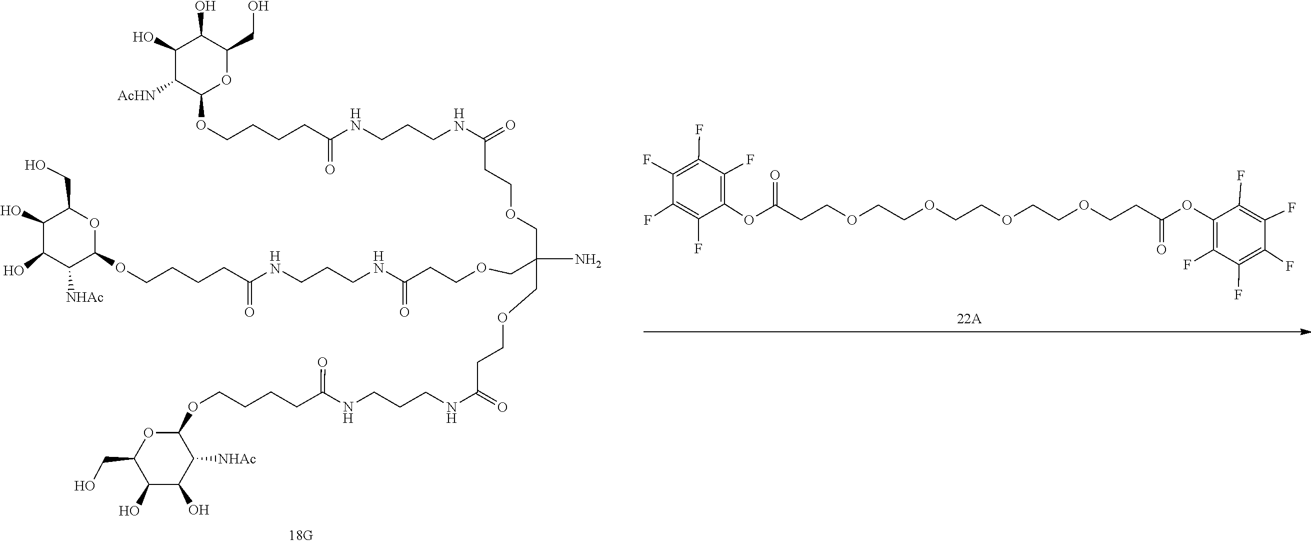 [SMILES: CC(=O)N[C@H]1[C@H](OCCCCC(=O)NCCCNC(=O)CCOCC(N)(COCCC(=O)NCCCNC(=O)CCCCO[C@@H]2O[C@H](CO)[C@H](O)[C@H](O)[C@H]2NC(C)=O)COCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O ; O=C(CCOCCOCCOCCOCCC(=O)Oc1c(F)c(F)c(F)c(F)c1F)Oc1c(F)c(F)c(F)c(F)c1F]

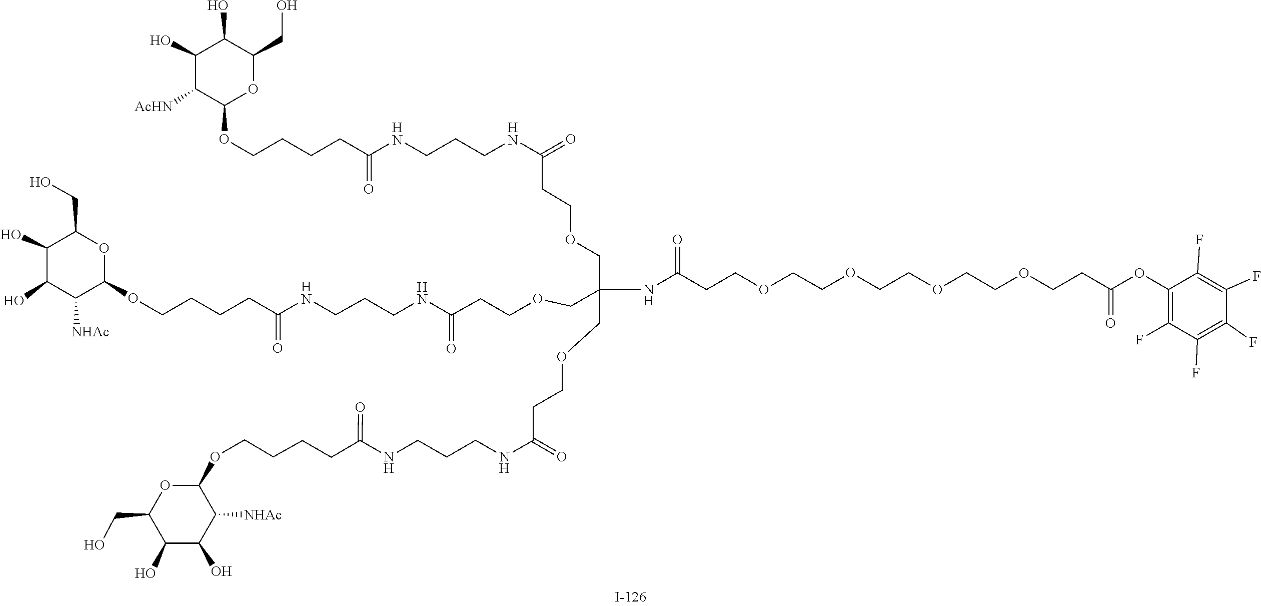 [SMILES: CC(=O)N[C@H]1[C@H](OCCCCC(=O)NCCCNC(=O)CCOCC(COCCC(=O)NCCCNC(=O)CCCCO[C@@H]2O[C@H](CO)[C@H](O)[C@H](O)[C@H]2NC(C)=O)(COCCC(=O)NCCCNC(=O)CCCCO[C@@H]2O[C@H](CO)[C@H](O)[C@H](O)[C@H]2NC(C)=O)NC(=O)CCOCCOCCOCCOCCC(=O)Oc2c(F)c(F)c(F)c(F)c2F)O[C@H](CO)[C@H](O)[C@@H]1O]

To a solution of Compound 18G (0.05 g, 0.035 mmol) and bis(2,3,4,5,6-pentafluorophenyl) 4,7,10,13-tetraoxahexadecanedioate (22A) 0.022 g, 0.035 mmol) in N,N-dimethylformamide (1.0 mL) was added N,N-diisopropylethylamine (0.031 mL, 0.177 mmol). The reaction mixture was stirred at room temperature for 12 h. The reaction mixture was diluted with acetonitrile, filtered and purified by prep HPLC (13-45% acetonitrile in water with 0.1% ammonium acetate). Fractions containing the desired product were combined and lyophilized to dryness to afford Compound I-126 as white solid. Yield: 0.009 g, 13.71%.MS (ESI) m/z, 1858 [M+1]⁺, 729 [M/2+1]⁺. ¹H NMR (400 MHz, DMSO-d₆) δ 7.83 (t, J=5.6 Hz, 3H), 7.35 (t, J=5.2 Hz, 3H), 7.61 (d, J=8.8 Hz, 3H), 7.13 (s, 1H), 4.59-4.54 (m, 6H), 4.46 (d, J=4.4 Hz, 3H), 4.21 (d, J=8.4 Hz, 3H), 3.76-3.70 (m, 2H), 3.67-3.63 (m, 10H), 3.55-3.46 (m, 34H), 3.14 (s, 2H), 3.32-3.28 (m, 2H), 3.02 (t, J=6 Hz, 16H), 2.27 (t, J=6 Hz, 6H), 2.03 (t, J=7.2 Hz, 6H), 1.79 (s, 9H), 1.51-139 (m, 20H).

### ASGPR Example 127: Synthesis of Compound I-127

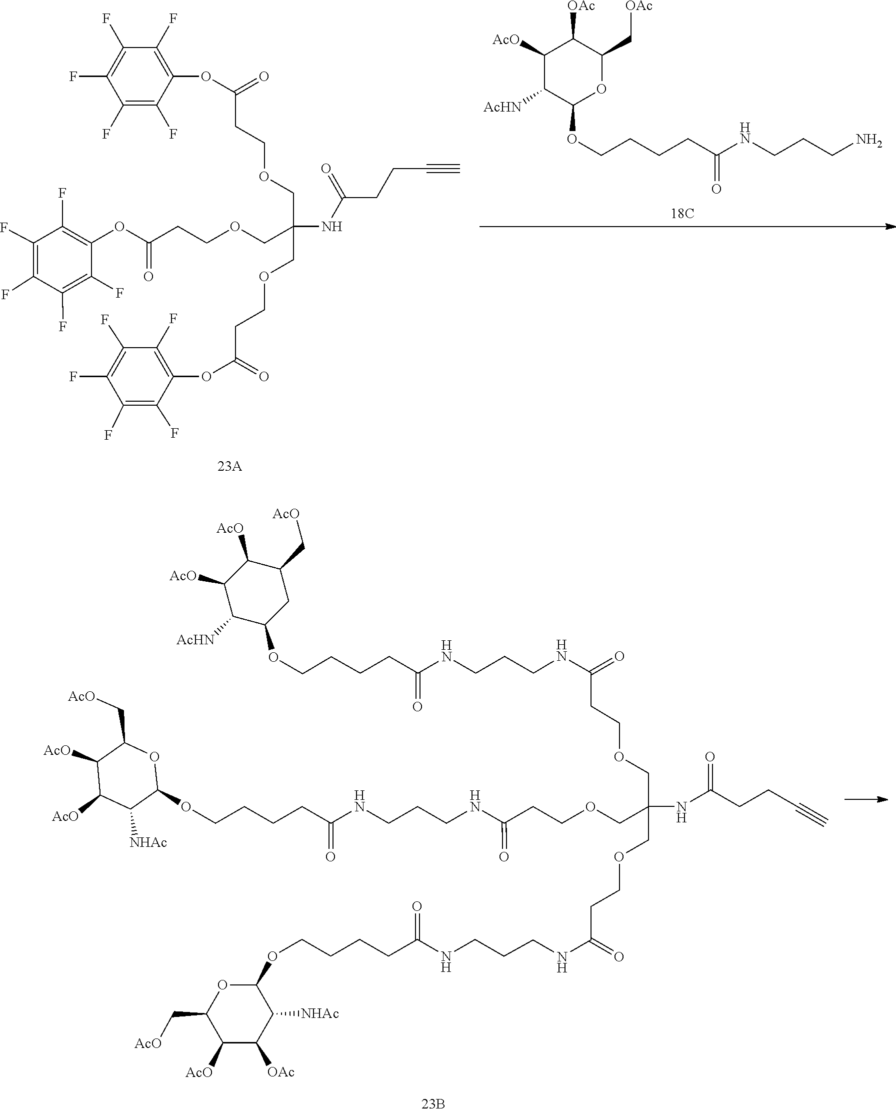 [SMILES: C#CCCC(=O)NC(COCCC(=O)Oc1c(F)c(F)c(F)c(F)c1F)(COCCC(=O)Oc1c(F)c(F)c(F)c(F)c1F)COCCC(=O)Oc1c(F)c(F)c(F)c(F)c1F ; CC(=O)N[C@H]1[C@H](OCCCCC(=O)NCCCN)O[C@H](COC(C)=O)[C@H](OC(C)=O)[C@@H]1OC(C)=O ; C#CCCC(=O)NC(COCCC(=O)NCCCNC(=O)CCCCO[C@@H]1O[C@H](COC(C)=O)[C@H](OC(C)=O)[C@H](OC(C)=O)[C@H]1NC(C)=O)(COCCC(=O)NCCCNC(=O)CCCCO[C@@H]1O[C@H](COC(C)=O)[C@H](OC(C)=O)[C@H](OC(C)=O)[C@H]1NC(C)=O)COCCC(=O)NCCCNC(=O)CCCCO[C@@H]1C[C@H](COC(C)=O)[C@H](OC(C)=O)[C@H](OC(C)=O)[C@H]1NC(C)=O]

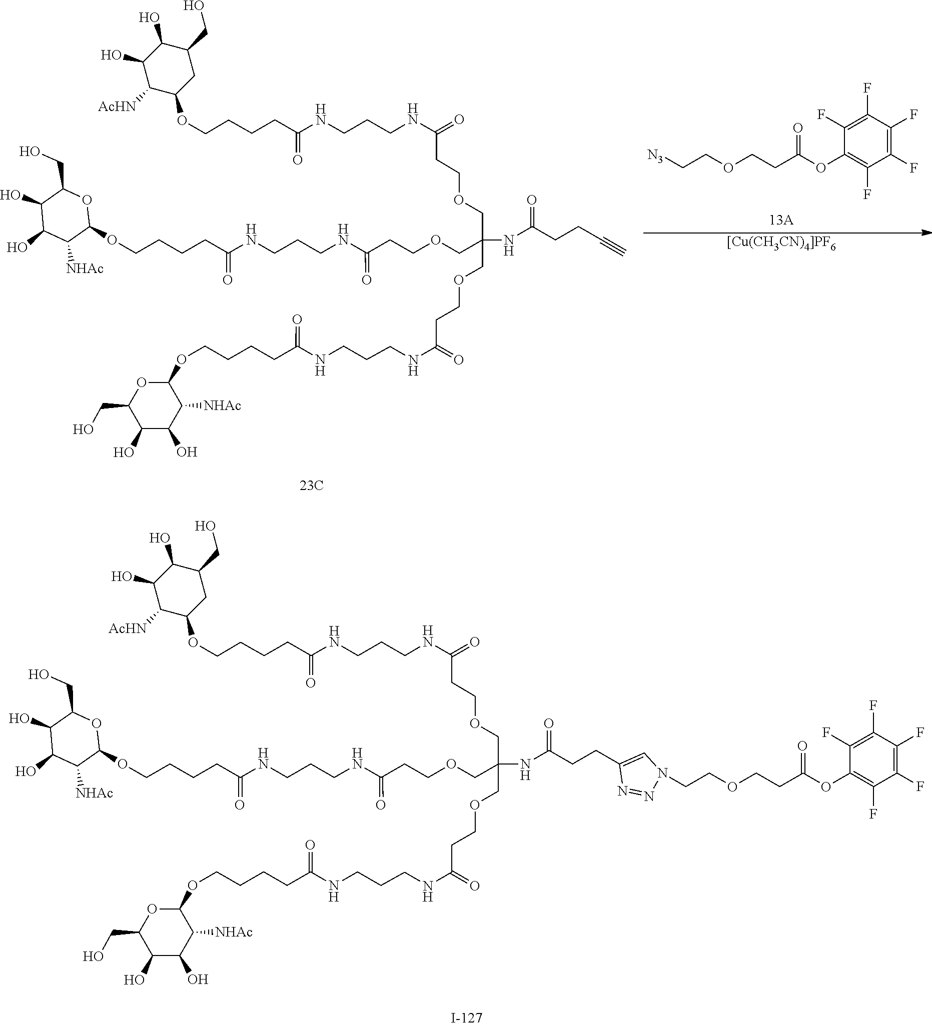

To a solution of bis(perfluorophenyl) 3,3′-((2-((3-oxo-3-(perfluorophenoxy)propoxy)methyl)-2-(pent-4-ynamido)propane-1,3-diyl)bis(oxy))dipropionate (23A) (1.0 eq, 0.500 g, 0.54 mmol) and Compound 18C (4.0 eq, 1.3 g, 2.16 mmol) in N,N-dimethylformamide (10 mL), N,N-diisopropylethylamine (6.0 eq, 0.59 mL, 3.24 mmol) was added and reaction mixture was stirred at room temperature for 16 h. After completion, reaction mixture was concentrated and dried to afford Compound 23B as a light brown viscous liquid. Yield: 3.0 g (Crude), ELSD m/z 937.4 [(M/2)+1]+.

To a solution of Compound 23B (1.0 eq, 3.0 g, 1.60 mmol) in methanol (10 mL), sodium methoxide (25% solution in methanol) (10.0 eq, 3.92 mL, 16.0 mmol) was added and reaction mixture was stirred at room temperature for 1 h. The reaction was monitored by ELSD. After completion, reaction mixture was neutralized with Dowex 50WX8 hydrogen form (200-400 mesh) and filtered. The filtrate was concentrated to afford crude which was diluted with acetonitrile and purified by prep HPLC (13-25% acetonitrile in water). Fractions containing the desired product were combined and lyophilized to dryness to afford Compound 23C as an off white solid. Yield: 0.380 g, 15.45%; LCMS m/z 748.35 [(M/2)+1]⁺.

To a solution of Compound 23C (1.0 eq, 0.040 g, 0.026 mmol) in dimethylsulfoxide (1.0 mL), Compound 13A (1.2 eq, 0.010 g, 0.032 mmol) was added and stirred for 5 minutes. Then, tetrakis(acetonitrile)copper(I) hexafluorophosphate (2.8 eq., 0.027 g, 0.074 mmol) was added and reaction mixture was stirred at room temperature for 15 minutes. After completion, reaction mixture was diluted with acetonitrile and purified by prep H PLC (20-45% acetonitrile in water with 0.1% TFA). Fractions containing the desired product were combined and lyophilized to dryness to afford Compound I-127 as an off white solid. Yield: 0.008 g, 16.6%; LCMS m/z 911.31 [(M/2)+1]⁺; ¹H NMR (400 MHz, D₂O) δ 7.71 (s, 1H), 4.57-4.54 (m, 3H), 4.39 (d, J=8.4 Hz, 4H), 3.94 (t, J=8.4 Hz, 2H), 3.91-3.79 (m, 10H), 3.76-3.72 (m, 5H), 3.69-3.67 (m, 2H), 3.65-3.63 (m, 10H), 3.58 (bs, 5H), 3.55-3.52 (m, 4H), 3.18-3.13 (m, 12H), 2.95 (t, J=5.2 Hz, 2H), 2.81 (t, J=6.8 Hz, 2H), 2.49-2.46 (m, 2H), 2.44-2.41 (m, 6H), 2.19-2.17 (m, 6H), 1.98 (s, 9H), 1.69-1.62 (m, 6H), 1.60-1.49 (m, 12H).

### ASGPR Example 128: Synthesis of Compound I-128

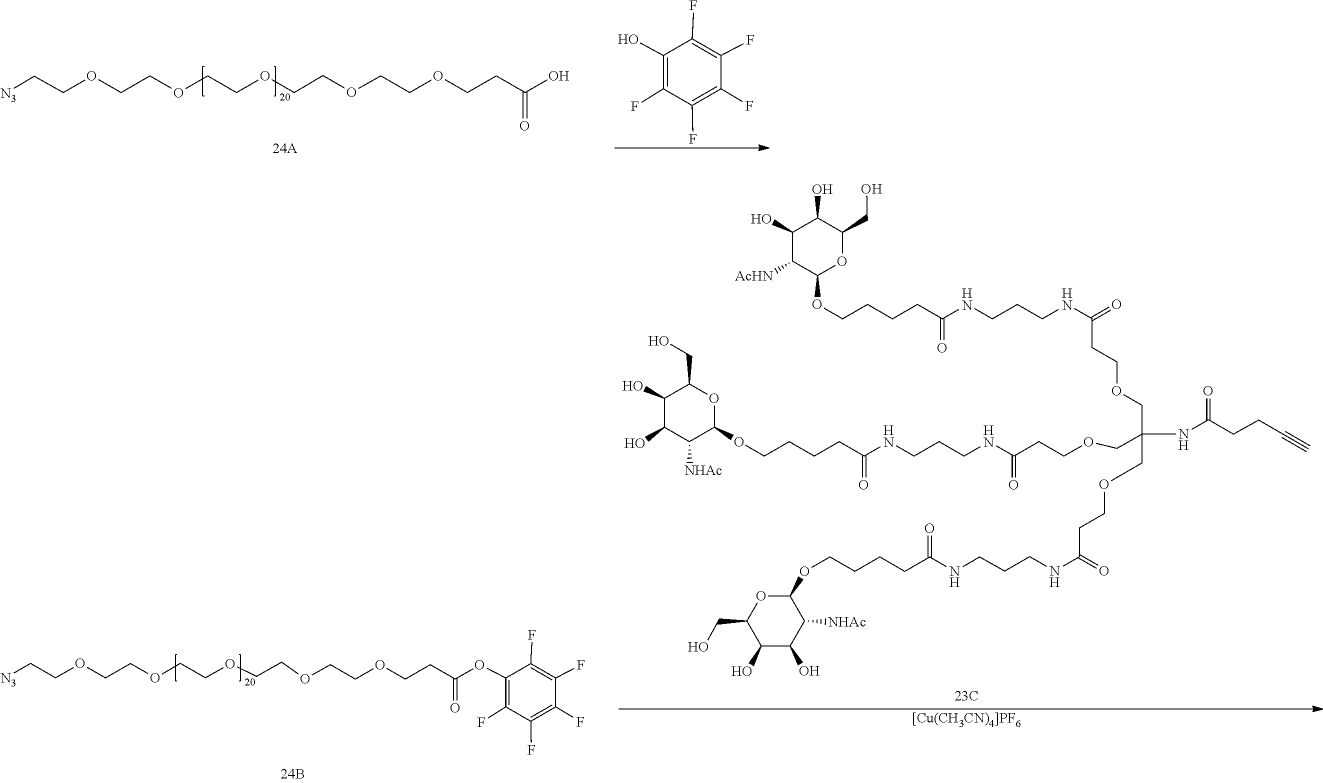

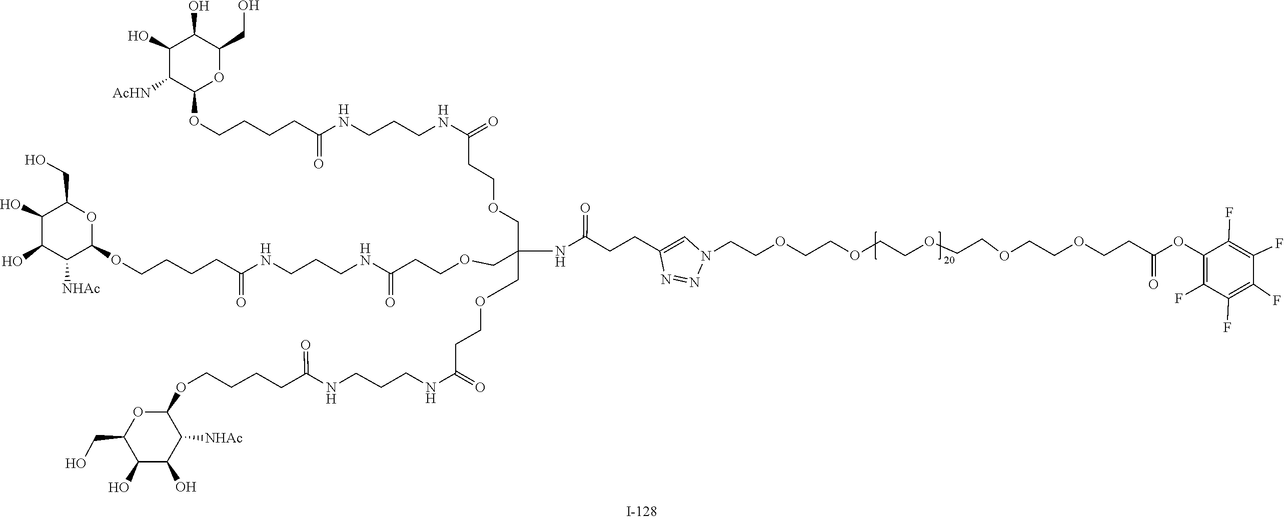

To a solution of 1-azido-3,6,9,12,15,18,21,24,27,30,33,36,39,42,45,48,51,54,57,60,63,66,69,72-tetracosaoxapentaheptacontan-75-oic acid (24A) (1.0 eq, 0.050 g, 0.042 mmol) in dichloromethane (1.0 mL), pentafluorophenol (1.1 eq, 0.008 g, 0.046 mmol) and N,N′-diisopropylcarbodiimide (1.5 eq, 0.008 g, 0.064 mmol) were added and reaction mixture was stirred at room temperature for 2 h. After completion, reaction mixture was diluted with dichloromethane, filtered through syringe filter, filtrate was concentrated and dried to afford Compound 24B as a colourless sticky solid. Yield: 0.070 g (Crude), LCMS m/z 669.8 [(M/2)+1]⁺.

To a solution of Compound 23C (1.0 eq, 0.030 g, 0.016 mmol) in dimethylsulfoxide (0.5 mL), Compound 24B (2.0 eq, 0.042 g, 0.032 mmol) was added and stirred for 5 minutes. Then, tetrakis(acetonitrile)copper(I) hexafluorophosphate (2.8 eq., 0.016 g, 0.044 mmol) was added and reaction mixture was stirred at room temperature for 15 minutes. After completion, reaction mixture was diluted with acetonitrile and purified by prep HPLC (27-62% acetonitrile in water with 0.1% TFA). Fractions containing the desired product were combined and lyophilized to dryness to afford Compound I-128 as a colourless sticky solid. Yield: 0.012 g, 25.93%; LCMS m/z 1417.18 [(M/2)+1]⁺; ¹H NMR (400 MHz, D₂O) δ 7.82 (s, 1H), 4.56 (bs, 3H), 4.40 (d, J=8.4 Hz, 4H), 3.90-3.82 (m, 14H), 3.75-3.70 (m, 5H), 3.67-3.49 (m, 111H), 3.17 (d, J=6.4 Hz, 12H), 3.05 (t, J=5.2 Hz, 2H), 2.92 (t, J=7.6 Hz, 2H), 2.57 (t, J=6.0 Hz, 2H), 2.43 (bs, 6H), 2.20 (bs, 6H), 1.99 (s, 9H), 1.71-1.66 (m, 6H), 1.54 (bs, 12H).

### ASGPR Example 129: Synthesis of Compound I-129

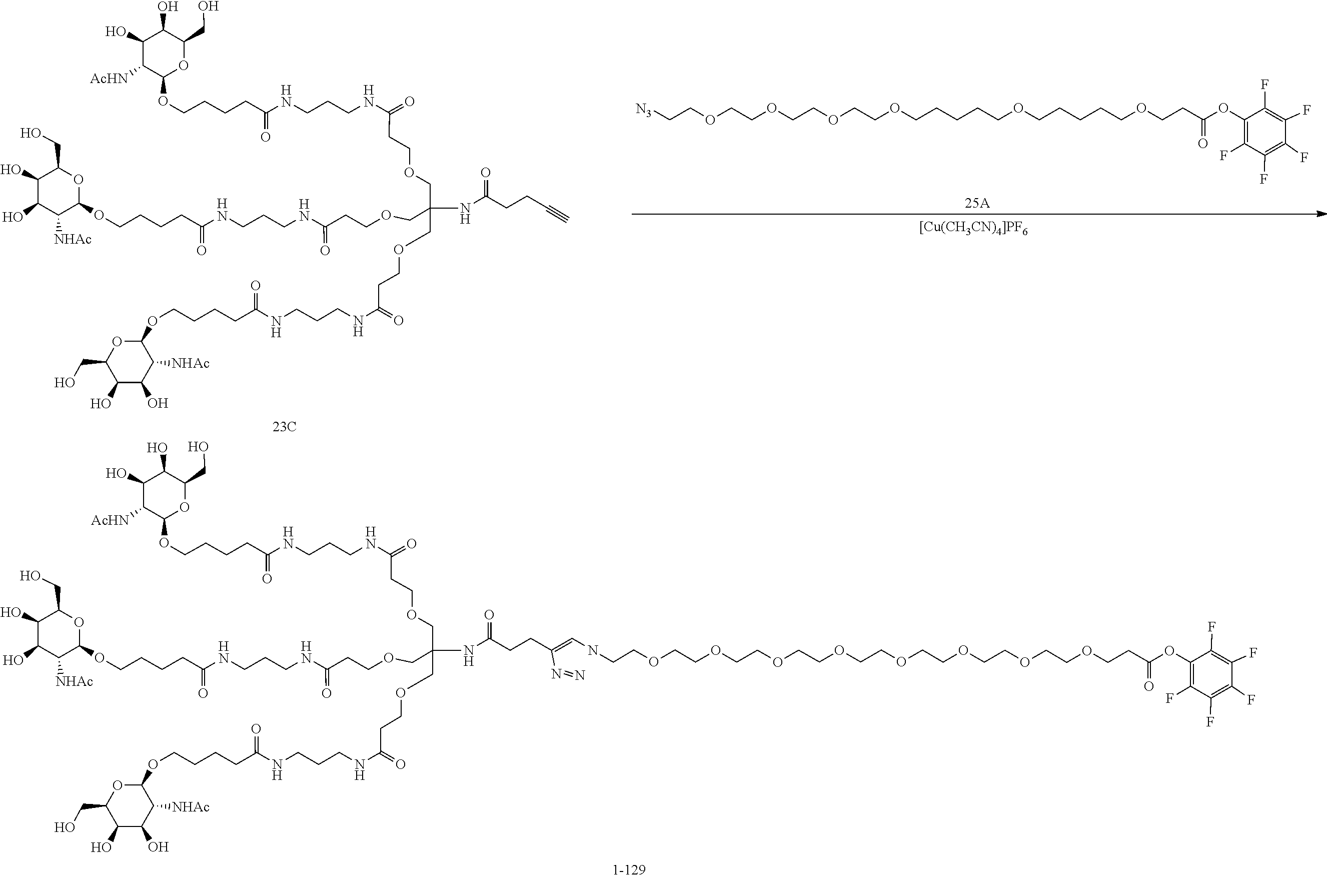

To a solution of Compound 23C (1.0 eq, 0.060 g, 0.040 mmol) in dimethylsulfoxide (1.0 mL), perfluorophenyl 1-azido-3,6,9,12,15,18,21,24-octaoxaheptacosan-27-oate (25A) (1.1 eq, 0.028 g, 0.044 mmol) was added and stirred for 5 minutes. Then, tetrakis(acetonitrile)copper(I) hexafluorophosphate (2.8 eq., 0.041 g, 0.112 mmol) was added and reaction mixture was stirred at room temperature for 1 h. After completion, reaction mixture was diluted with acetonitrile and purified by prep HPLC (27-58% acetonitrile in water with 0.1% TFA). Fractions containing the desired product were combined and lyophilized to dryness to afford Compound I-129 as a colourless sticky solid. Yield: 0.006 g, 6.44%; LCMS m/z 1065.25 [(M/2)+1]⁺; ¹H NMR (400 MHz, D₂O) δ 7.81 (s, 1H), 4.55 (bs, 2H), 4.39 (d, J=8.4 Hz, 3H), 3.89-3.82 (m, 12H), 3.78-3.74 (m, 5H), 3.71-3.58 (m, 51H), 3.19-3.14 (m, 12H), 3.04 (t, J=5.2 Hz, 2H), 2.91 (t, J=7.2 Hz, 2H), 2.56 (t, J=7.6 Hz, 2H), 2.42 (bs, 6H), 2.21-2.10 (m, 6H), 1.98 (s, 9H), 1.66 (t, J=6.8 Hz, 6H), 1.53 (bs, 12H).

### ASGPR Example 130: Synthesis of Compound I-130

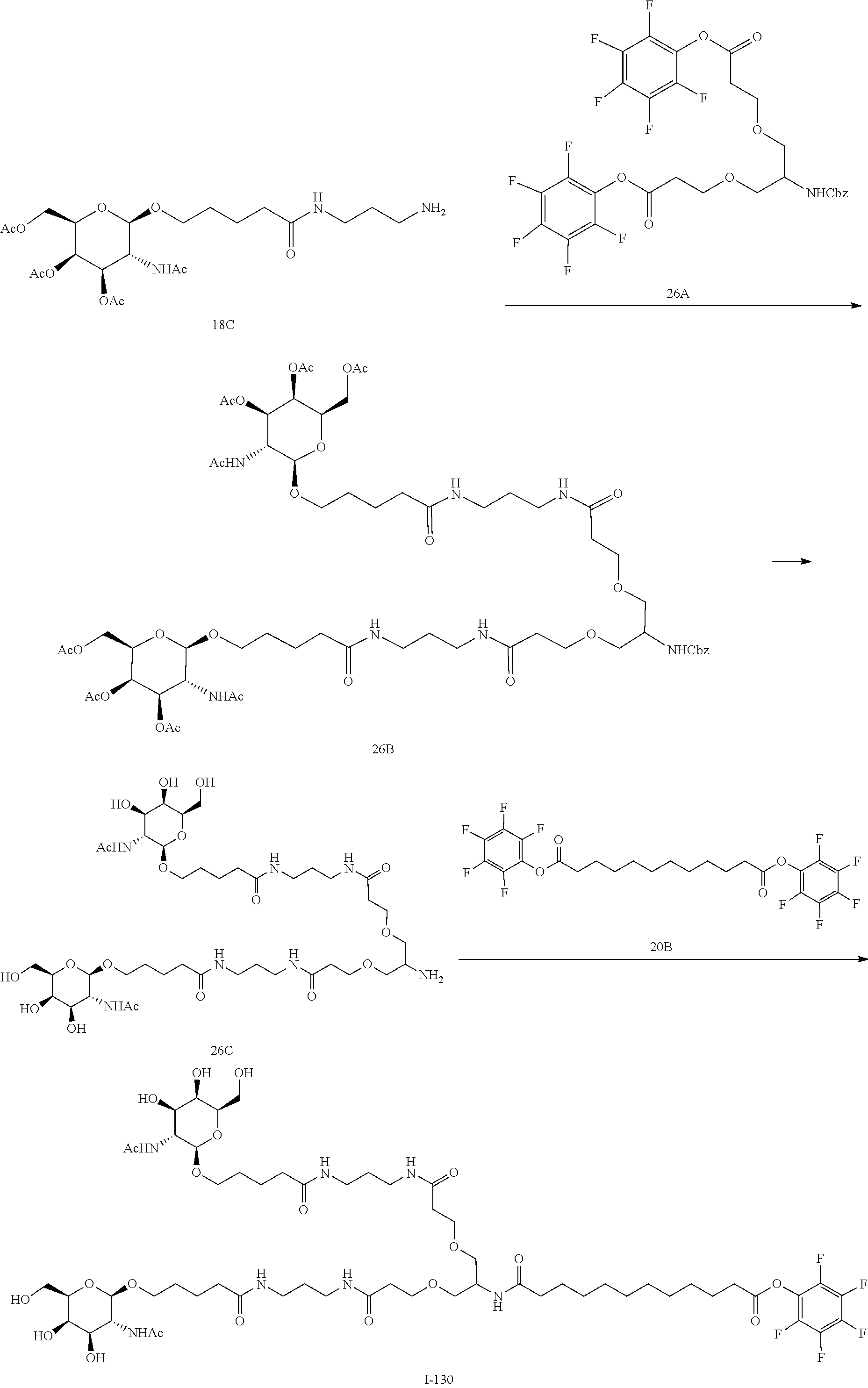

Compound 26B is synthesized by employing the procedures described for Compound 18E using Compound 26A in lieu of Compound 18D.

To a stirred solution of Compound 26B and acetic acid (1.0 eq) in methonal 20% palladium on carbon (10%) is added at 0° C. The resulting mixture is stirred at 0° C. and warmed to room temperature under hydrogen gas for 3 h. The reaction mixture is filtered through Celite bed and washed with methoanl, filtrate concentrated under vaccum to afford Compound 26C.

Compound I-130 is synthesized by employing the procedures described for Compounds 1-122 using Compounds 26C and 20B in lieu of Compounds 18G and 3E.

### ASGPR Example 131: Synthesis of Compound I-131

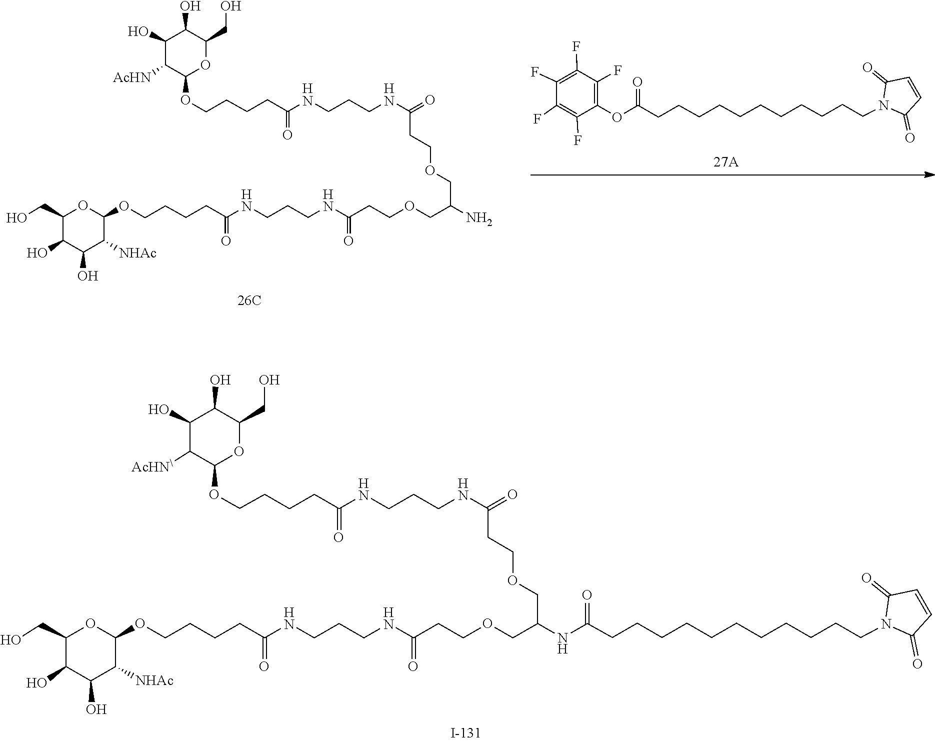

Compound I-131 is synthesized by employing the procedures described for Compound I-130 using Compound 27A in lieu of Compound 20B.

### ASGPR Example 132: Synthesis of Compound I-132

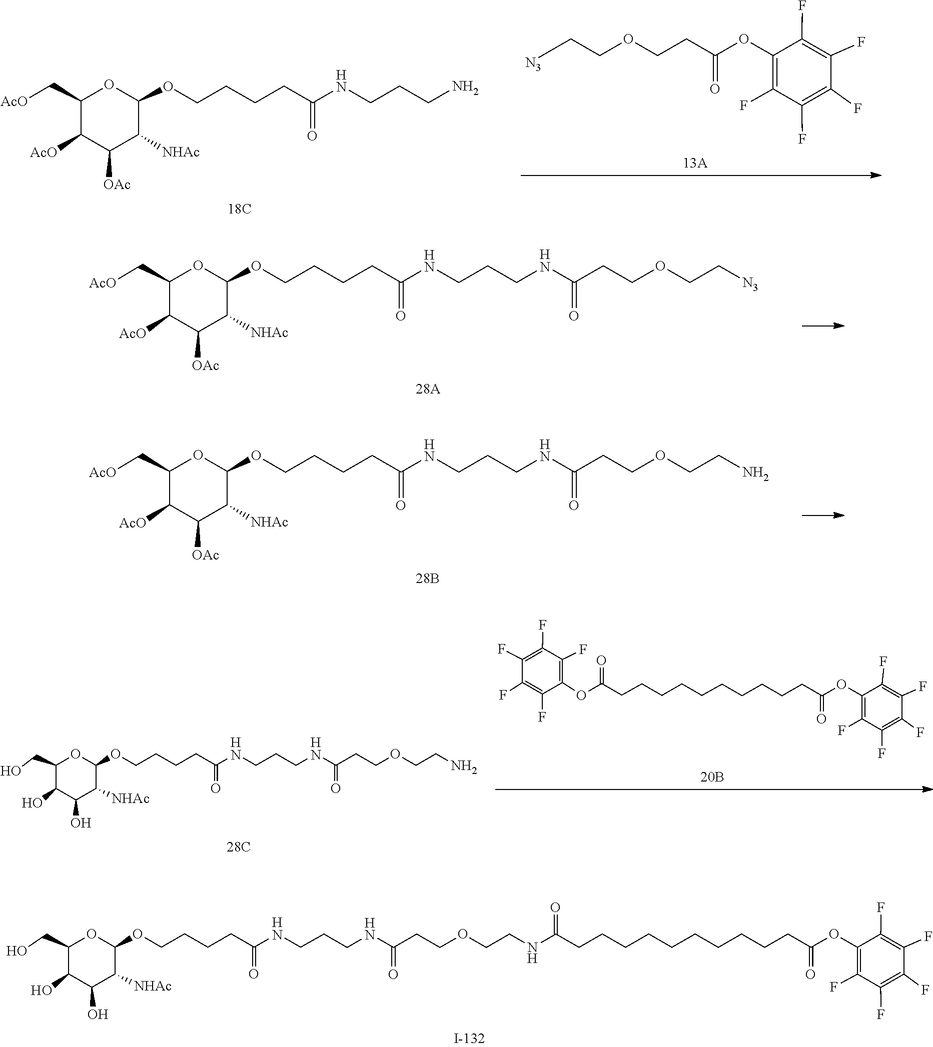

Compound 28A is synthesized by employing the procedures described for Compound I-123 using Compounds 18C and 13A in lieu of Compounds 18G and 19C.

To a stirred solution of Compound 28B in methonal 20% palladium on carbon (0.05 g) is added at room temperature. The resulting mixture is stirred at room temperature under hydrogen gas for 16 h. The reaction mixture is filtered through Celite bed and washed with methoanl, filtrate concentrated under vaccum to afford Compound 28B.

Compounds 28C and 1-132 are synthesized by employing the procedures described for Compounds 26C and 1-26 using Compound 28B and 28C in lieu of Compound 26B and 26C.

### ASGPR Example 133A: Synthesis of Compound I-133

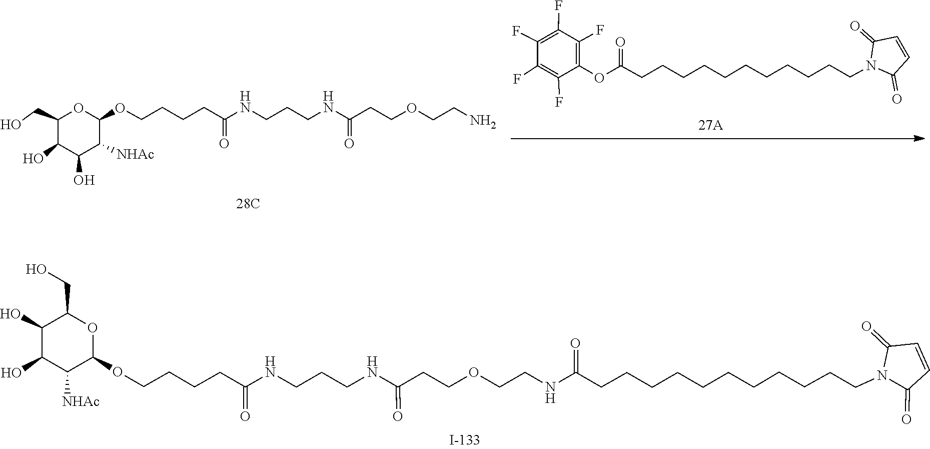 [SMILES: O=C(CCCCCCCCCCCN1C(=O)C=CC1=O)Oc1c(F)c(F)c(F)c(F)c1F ; CC(=O)N[C@H]1[C@H](OCCCCC(=O)NCCCNC(=O)CCOCCN)O[C@H](CO)[C@H](O)[C@@H]1O ; CC(=O)N[C@H]1[C@H](OCCCCC(=O)NCCCNC(=O)CCOCCNC(=O)CCCCCCCCCCCN2C(=O)C=CC2=O)O[C@H](CO)[C@H](O)[C@@H]1O]

Compound I-133 is synthesized by employing the procedures described for Compound I-131 using Compound 28C in lieu of Compound 26C.

### ASGPR Example 133B: Synthesis of N-(2-(3-((3-(5-(((2R,3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)ethyl)-12-(2,5-dioxo-2,5-dihydro-1H-pyrrol-1-yl)dodecanamide (Compound I-133)

Alternatively, Compound I-133 is synthesized by the following procedure.

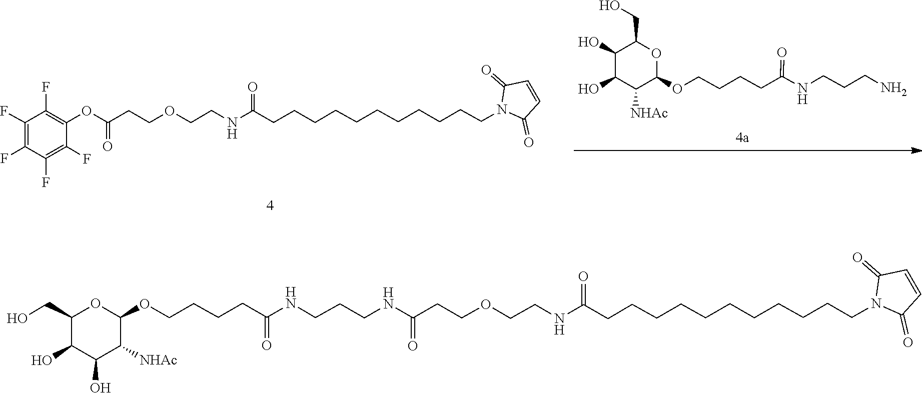 [SMILES: O=C(CCCCCCCCCCCN1C(=O)C=CC1=O)NCCOCCC(=O)Oc1c(F)c(F)c(F)c(F)c1F ; CC(=O)N[C@H]1[C@H](OCCCCC(=O)NCCCN)O[C@H](CO)[C@H](O)[C@@H]1O ; CC(=O)N[C@H]1[C@H](OCCCCC(=O)NCCCNC(=O)CCOCCNC(=O)CCCCCCCCCCCN2C(=O)C=CC2=O)O[C@H](CO)[C@H](O)[C@@H]1O]

### Synthesis of tert-butyl 3-(2-(12-(2,5-dioxo-2,5-dihydro-1H-pyrrol-1-yl)dodecanamido)ethoxy)propanoate

To a stirred solution of tert-butyl 3-(2-aminoethoxy)propanoate (0.20 g, 1.0 eq. 1.06 mmol) in acetonitrile (3.00 mL), perfluorophenyl 12-(2,5-dioxo-2,5-dihydro-1H-pyrrol-1-yl)dodecanoate (0.488 g, 1.0 eq, 1.06 mmol) was added at 0° C. and stirred for 3 h at room temperature. The reaction mixture was then concentrated and purified by flash column chromatography using 40% ethyl acetate in hexane to afford tert-butyl 3-(2-(12-(2,5-dioxo-2,5-dihydro-1H-pyrrol-1-yl)dodecanamido)ethoxy)propanoate as off white solid. Yield: 0.30 g, 60.0%. LCMS; m/z 467.3 [M+1]⁺.

### Synthesis of 3-(2-(12-(2,5-dioxo-2,5-dihydro-1H-pyrrol-1-yl)dodecanamido)ethoxy)propanoic acid

To stirred a solution of tert-butyl 3-(2-(12-(2,5-dioxo-2,5-dihydro-1H-pyrrol-1-yl)dodecanamido)ethoxy)propanoate (0.10 g, 1.0 eq., 0.214 mmol) in dichloromethane (1.00 mL) was added trifluoroacetic acid (1.00 mL) at 0° C. The reaction mixture was stirred at room temperature for 16 h., The reaction mixture was then concentrated under reduced pressure to get crude 3-(2-(12-(2,5-dioxo-2,5-dihydro-1H-pyrrol-1-yl)dodecanamido)ethoxy)propanoic acid (3) as a colourless liquid. The crude was proceeded as such for next step. Yield: 0.07 g (crude). LCMS; m/z 411.3 [M+1]⁺.

### Synthesis of perfluorophenyl 3-(2-(12-(2,5-dioxo-2,5-dihydro-1H-pyrrol-1-yl)dodecanamido)ethoxy)propanoate (4)

To stirred a solution of 3-(2-(12-(2,5-dioxo-2,5-dihydro-1H-pyrrol-1-yl)dodecanamido)ethoxy)propanoic acid (0.070 g, 1.0 eq., 0.171 mmol) in tetrahydrofuran (1.00 mL), pentafluorophenol (0.031 g, 1.0 eq., 0.171 mmol) and N,N′-diisopropylcarbodiimide (0.043 g, 2 eq., 0.341 mmol) was added at 0° C. and stirred for 1 h at room temperature. The reaction mixture was then concentrated to get the crude which was purified by flash column chromatography using silica gel column (eluting with 20% ethyl acetate in dichloromethane) to afford perfluorophenyl 3-(2-(12-(2,5-dioxo-2,5-dihydro-1H-pyrrol-1-yl)dodecanamido)ethoxy)propanoate (4) as white solid (Yield: 0.070 g, 71.0%); LCMS; m/z 577.02 [M+1]⁺.

### Synthesis of N-(2-(3-((3-(5-(((2R,3R,4R,5R,6R)-3-acetamido-4,5-dihydroxy (hydroxymethyl)tetrahydro-2H-pyran-2-yl)oxy)pentanamido)propyl)amino) oxopropoxy)ethyl)-12-(2,5-dioxo-2,5-dihydro-1H-pyrrol-1-yl)dodecanamide (Compound I-133)

To a stirred solution of perfluorophenyl 3-(2-(12-(2,5-dioxo-2,5-dihydro-1H-pyrrol-1-yl)dodecanamido)ethoxy)propanoate (4, 0.070 g, 1.0 eq, 0.121 mmol) in dimethyl sulfoxide (1.0 mL) was added 5-(((2R,3R,4R,5R,6R)-3-acetamido-4,5-dihydroxy-6-(hydroxymethyl)tetrahydro-2H-pyran-2-yl)oxy)-N-(3-aminopropyl)pentanamide (4a, 0.045 g, 1.0 eq., 0.121 mmol) at 0° C. and reaction mixture was stirred for 20 min at room temperature. The reaction mixture was then purified by prep-HPLC (50 to 60% acetonitrile in water using 0.1% TFA buffer) to afford N-(2-(3-((3-(5-(((2R,3R,4R,5R,6R)-3-acetamido-4,5-dihydroxy-6-(hydroxymethyl)tetrahydro-2H-pyran-2-yl)oxy)pentanamido)propyl)amino)-3-oxopropoxy)ethyl)-12-(2,5-dioxo-2,5-dihydro-1H-pyrrol-1-yl)dodecanamide (Compound I-133) as white solid; Yield:0.010 g, 10.7%), LC-MS; m/z 770.43 [M+1]⁺. ¹H NMR (400 MHz, DMSO-d₆ with D₂O exchange) δ 6.93 (s, 2H), 4.19 (d, J=8.4 Hz, 1H), 3.60-3.57 (m, 2H), 3.57 (t, J=6.4 Hz, 2H), 3.53-3.44 (m, 2H), 3.40-3.27 (m, 7H), 3.15-3.13 (m, 2H), 3.01 (brs, 4H), 2.28 (t, J=6 Hz, 2H), 2.02 (br t, J=7 Hz, 4H), 1.78 (s, 3H), 1.46 (m, 9H), 1.18 (br m, 15H).

### ASGPR Example 135: perfluorophenyl 1-(4-(3-(((2R,3R,4R,5R,6R)-3-acetamido-4,5-dihydroxy-6-(hydroxymethyl)tetrahydro-2H-pyran-2-yl)oxy)propyl)-1H-1,2,3-triazol-1-yl)-13,13-bis(3-((2-(2-(2-(4-(3-(((2R,3R,4R,5R,6R)-3-acetamido-4,5-di hydroxy-6-(hydroxymethyl)tetrahydro-2H-pyran-2-yl)oxy)propyl)-1H-1,2,3-triazol-1-yl)ethoxy)ethoxy)ethyl)am no)-3-oxopropyl)-10,15-dioxo-3,6-dioxa-9,14-diazahexacosan-26-oate (Cod. No. I-1351

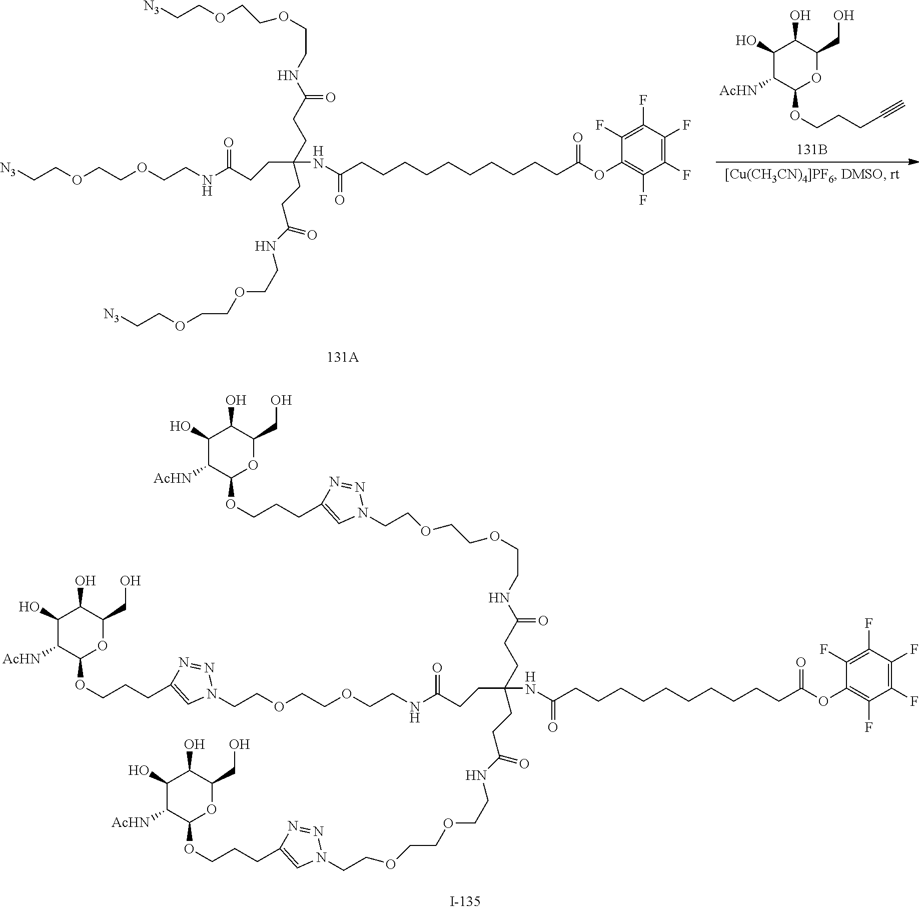 [SMILES: [N-]=[N+]=NCCOCCOCCNC(=O)CCC(CCC(=O)NCCOCCOCCN=[N+]=[N-])(CCC(=O)NCCOCCOCCN=[N+]=[N-])NC(=O)CCCCCCCCCCC(=O)Oc1c(F)c(F)c(F)c(F)c1F ; C#CCCCO[C@@H]1O[C@H](CO)[C@H](O)[C@H](O)[C@H]1NC(C)=O ; CC(=O)N[C@H]1[C@H](OCCCc2cn(CCOCCOCCNC(=O)CCC(CCC(=O)NCCOCCOCCn3cc(CCCO[C@@H]4O[C@H](CO)[C@H](O)[C@H](O)[C@H]4NC(C)=O)nn3)(CCC(=O)NCCOCCOCCn3cc(CCCO[C@@H]4O[C@H](CO)[C@H](O)[C@H](O)[C@H]4NC(C)=O)nn3)NC(=O)CCCCCCCCCCC(=O)Oc3c(F)c(F)c(F)c(F)c3F)nn2)O[C@H](CO)[C@H](O)[C@@H]1O]

To a solution of perfluorophenyl 1-azido-13,13-bis(3-((2-(2-(2-azidoethoxy)ethoxy)ethyl)amino)-3-oxopropyl)-10,15-dioxo-3,6-dioxa-9,14-diazahexacosan-26-oate (131A, 1.0 eq, 0.095 g, 0.086 mmol) in dimethylsulfoxide (2.0 mL), N-((2R,3R,4R, 5R,6R)-4,5-dihydroxy-6-(hydroxymethyl)-2-(pent-4-yn-1-yloxy)tetrahydro-2H-pyran-3-yl)acetamide (131B, 3.0 eq, 0.074 g, 0.26 mmol) was added and stirred for 5 minutes. Then, tetrakis(acetonitrile)copper(I) hexafluorophosphate (8.4 eq., 0.272 g, 0.729 mmol) was added and reaction mixture was stirred at room temperature for 1 h. After completion, reaction mixture was diluted with acetonitrile and purified by prep HPLC (33-53% acetonitrile in water with 0.1% TFA). Fractions containing the desired product were combined and lyophilized to dryness to afford perfluorophenyl 1-(4-(3-(((2R,3R,4R,5R,6R)-3-acetamido-4,5-dihydroxy-6-(hydroxymethyl)tetrahydro-2H-pyran-2-yl)oxy)propyl)-1H-1,2,3-triazol-1-yl)-13,13-bis(3-((2-(2-(2-(4-(3-(((2R,3R,4R,5R,6R)-3-acetamido-4,5-dihydroxy-6-(hydroxymethyl)tetrahydro-2H-pyran-2-yl)oxy)propyl)-1H-1,2,3-triazol-1-yl)ethoxy)ethoxy)ethyl)amino)-3-oxopropyl)-10,15-dioxo-3,6-dioxa-9,14-diazahexacosan-26-oate (Cpd. No. I-135) as an off white solid. Yield: 0.036 g, 19.2%; LCMS m/z 978.89 [(M/2)+1]⁺; ¹H NMR (400 MHz, DMSO-d₆ with D₂O) δ 7.76 (s, 3H), 4.42 (t, J=5.2 Hz, 6H), 4.22 (d, J=8.8 Hz, 3H), 3.75-3.68 (m, 11H), 3.63-3.62 (m, 3H), 3.54-3.46 (m, 13H), 3.43-3.42 (m, 8H), 3.40-3.37 (m, 4H), 3.35-3.24 (m, 10H), 3.12 (t, J=5.6 Hz, 6H), 2.71 (t, J=7.2 Hz, 2H), 2.61-2.57 (m, 6H), 2.05-1.92 (m, 7H), 1.79 (s, 9H), 1.76-1.73 (m, 10H), 1.62-1.60 (m, 2H), 1.45-1.41 (m, 2H), 1.36-1.29 (m, 2H), 1.25-1.16 (m, 10H).

### ASGPR Example 136: perfluorophenyl 1-(4-((((2R,3R,4R,5R,6R)-5-acetamido-3,4-dihydroxy-6-methoxytetrahydro-2H-pyran-2-yl)methoxy)methyl)-1H-1,2,3-triazol-1-yl)-13,13-bis(3-((2-(2-(2-(4-((((2R,3R,4R,5R,6R)-5-acetamido-3,4-dihydroxy-6-methoxytetrahydro-2H-pyran-2-yl)methoxy)methyl)-1H-1,2,3-triazol yl)ethoxy)ethoxy)ethyl)amino)-3-oxopropyl)-10,15-dioxo-3,6-dioxa-9,14-diazahexacosan-26-oate (Cpd. No. I-136)

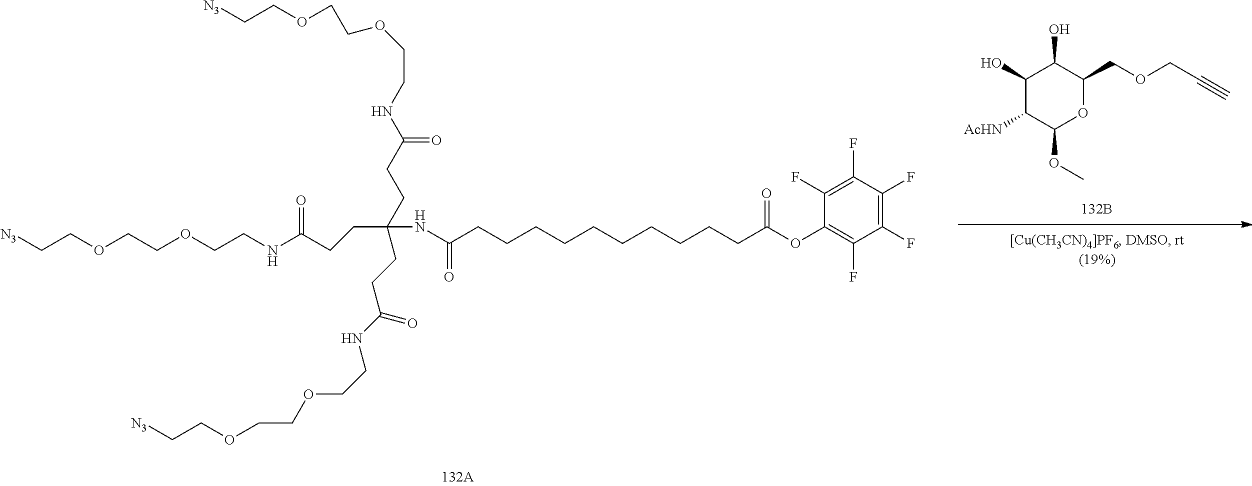 [SMILES: [N-]=[N+]=NCCOCCOCCNC(=O)CCC(CCC(=O)NCCOCCOCCN=[N+]=[N-])(CCC(=O)NCCOCCOCCN=[N+]=[N-])NC(=O)CCCCCCCCCCC(=O)Oc1c(F)c(F)c(F)c(F)c1F ; C#CCOC[C@H]1O[C@@H](OC)[C@H](NC(C)=O)[C@@H](O)[C@H]1O]

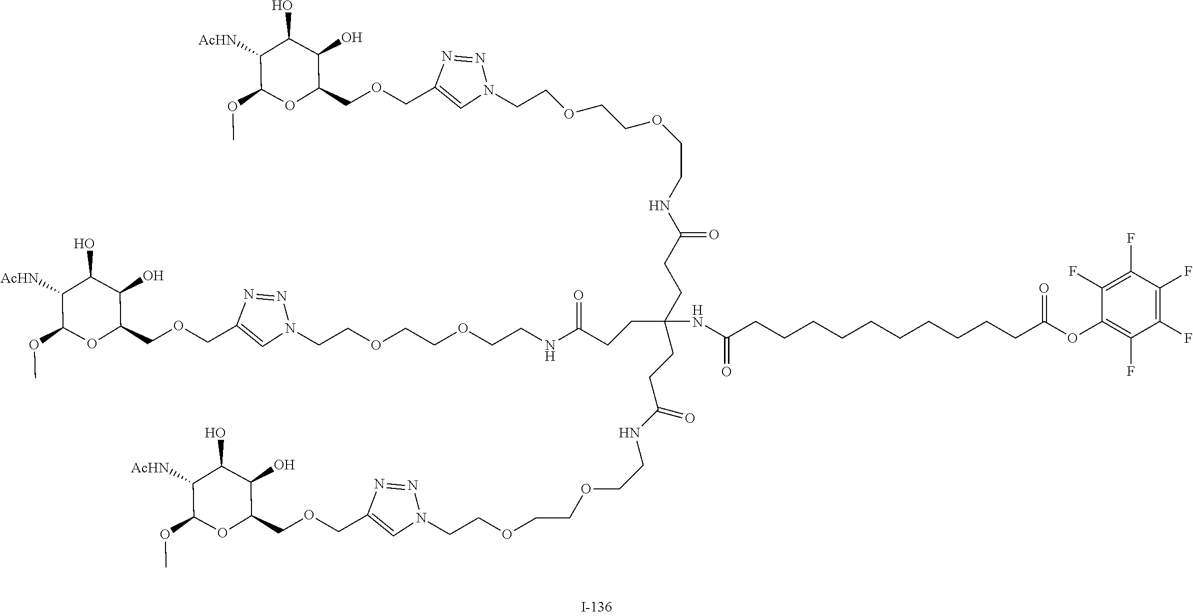 [SMILES: CO[C@@H]1O[C@H](COCc2cn(CCOCCOCCNC(=O)CCC(CCC(=O)NCCOCCOCCn3cc(COC[C@H]4O[C@@H](OC)[C@H](NC(C)=O)[C@@H](O)[C@H]4O)nn3)(CCC(=O)NCCOCCOCCn3cc(COC[C@H]4O[C@@H](OC)[C@H](NC(C)=O)[C@@H](O)[C@H]4O)nn3)NC(=O)CCCCCCCCCCC(=O)Oc3c(F)c(F)c(F)c(F)c3F)nn2)[C@H](O)[C@H](O)[C@H]1NC(C)=O]

To a solution of perfluorophenyl 1-azido-13,13-bis(3-((2-(2-(2-azidoethoxy)ethoxy)ethyl)amino)-3-oxopropyl)-10,15-dioxo-3,6-dioxa-9,14-diazahexacosan-26-oate (132A, 1.0 eq, 0.160 g, 0.146 mmol) in dimethyl sulfoxide (3 mL), N-((2R,3R,4R,5R,6R)-4,5-dihydroxy-2-methoxy-6-((prop-2-yn-1-yloxy)methyl)tetrahydro-2H-pyran-3-yl)acetamide (132B, 3.0 eq, 0.120 g, 0.439 mmol) and tetrakis(acetonitrile)copper(I) hexafluorophosphate (8.4 eq, 0.458 g, 1.23 mmol) were added and reaction mixture was stirred at room temperature for 1 h. After completion, reaction mixture was diluted with acetonitrile and purified by prep HPLC (eluting from a C18 column with 30-57% acetonitrile in water with 0.1% TFA). Fractions containing the desired product were combined and lyophilized to dryness to afford perfluorophenyl 1-(4-((((2R,3R,4R,5R,6R)-5-acetamido-3,4-dihydroxy-6-methoxytetrahydro-2H-pyran-2-yl)methoxy)methyl)-1H-1,2,3-triazol-1-yl)-13,13-bis(3-((2-(2-(2-(4-((((2R,3R,4R,5R,6R)-5-acetamido-3,4-dihydroxy-6-methoxytetrahydro-2H-pyran-2-yl)methoxy)methyl)-1H-1,2,3-triazol-1-yl)ethoxy)ethoxy)ethyl)amino)-3-oxopropyl)-10,15-dioxo-3,6-dioxa-9,14-diazahexacosan-26-oate (Cpd. No. I-136) as an off white solid. Yield: 0.055 g, 19.6%; LCMS m/z 957.74 [(M/2)+1]⁺; ¹H NMR (400 MHz, DMSO-d₆) δ 8.04 (s, 3H), 7.81-7.80 (m, 2H), 7.63 (d, J=8.8 Hz, 2H), 7.11 (s, 1H), 4.54 (d, J=4.4 Hz, 5H), 4.50 (t, J=5.2 Hz, 6H), 4.16 (d, J=8.4 Hz, 3H), 3.80 (t, J=5.2 Hz, 8H), 3.76-3.69 (m, 4H), 3.63-3.56 (m, 12H), 3.52-3.49 (m, 14H), 3.47-3.44 (m, 11H), 3.29 (s, 9H), 3.20 (s, 1H), 3.15 (d, J=6.0 Hz, 8H), 2.76 (t, J=6.8 Hz, 2H), 2.03-1.96 (m, 9H), 1.83-1.76 (m, 11H), 1.71-1.63 (m, 2H), 1.45-1.40 (m, 2H), 1.36-1.32 (m, 2H), 1.28-1.20 (m, 12H).

### CONJUGATION EXAMPLES

### Example 137: Conjugation of Isothiocyanate-Based Ligand-Linker Compounds with Anti-EGFR and Anti-PD-L1 Antibodies

This example provides a general protocol for the conjugation of the isothiocyanate-based ligand-linker compounds (e.g., Compound A) with the primary amines on lysine residues of anti-EGFR antibodies (e.g., matuzumab, cetuximab) and anti-PD-L1 antibodies (e.g., atezolizumab, anti-PD-L1(29E.2A3)). The conjugates thus obtained are listed in Table 2.

The antibody was buffer exchanged into 100 mM sodium bicarbonate buffer pH 9.0 at 5 mg/mL concentration, after which about 30 equivalents of the isothiocyanate-based ligand-linker compound (e.g., Compound A; freshly prepared as 20 mM stock solution in DMSO) was added and incubated overnight at ambient temperature in a tube revolver at 10 rpm.

The conjugates containing on average eight ligand-linker moieties per antibody were purified using a PD-10 desalting column (GE Healthcare) and followed with formulating the final conjugate into PBS pH 7.4 with Amicon Ultra 15 mL Centrifugal Filters with 30 kDa molecular weight cutoff.

### Example 138: Conjugation of Periluorophenoxy-Based Ligand-Linker Compounds with Anti-EGFR and IgG Antibodies

This example provides a general protocol for the conjugation of the perfluorophenoxy-based ligand-linker compounds (e.g., Compound I-7) with the primary amines on lysine residues of anti-EGFR antibodies (e.g., matuzumab, cetuximab) and IgG antibodies (e.g., IgG2a-UNLB). The conjugates thus obtained are listed in Table 2.

The antibody was buffer exchanged into 50 mM sodium phosphate buffer pH 8.0 at 5 mg/mL concentration, after which about 22 equivalents of perfluorophenoxy-based ligand-linker compound (e.g., Compound I-7; freshly prepared as 20 mM stock solution in DMSO) was added and incubated for 3 hours at ambient temperature in a tube revolver at 10 rpm.

The conjugates containing on average eight ligand-linker moieties per antibody were purified using a PD-10 desalting column (GE Healthcare) and followed with formulating the final conjugate into PBS pH 7.4 with Amicon Ultra 15 mL Centrifugal Filters with 30 kDa molecular weight cutoff.

### Example 139: Determination of DAR Values by Mass Spectrometry

This example provides the method for determining DAR values for the conjugates prepared as described in Examples 137 and 138. To determine the DAR value, 10 pg of the antibody (unconjugated or conjugated) was treated 2 μL of non-reducing denaturing buffer (10×, New England Biolabs) for 10 minutes at 75° C. The denatured antibody solution was then deglycosylated by adding 1.5 μL of Rapid-PNGase F (New England Biolabs) and incubated for 10 minutes at 50° C. Deglycosylated samples were diluted 50-fold in water and analyzed on a Waters ACQUITY UPLC interfaced to Xevo G2-S QToF mass spectrometer. Deconvoluted masses were obtained using Waters MassLynx 4.2 Software. DAR values were calculated using a weighted average of the peak intensities corresponding to each loading species using the formula below:

DAR=Σ(drug load distribution (%) of each Ab with drug load n)(n)/100

DAR values for the conjugates prepared as described in Examples 137 and 138 are shown in Table 10.

### Example 140: Determination of Purity of Conjugates by SEC Method

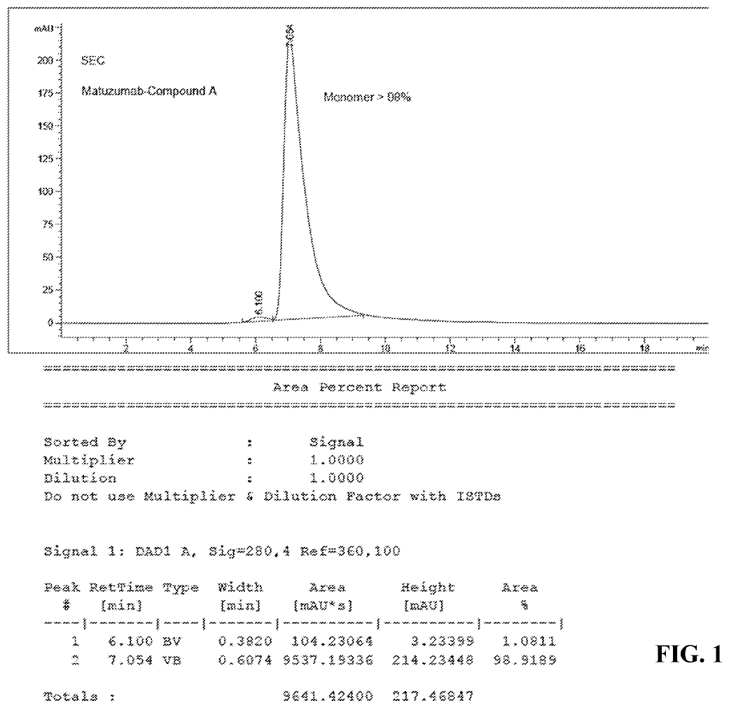

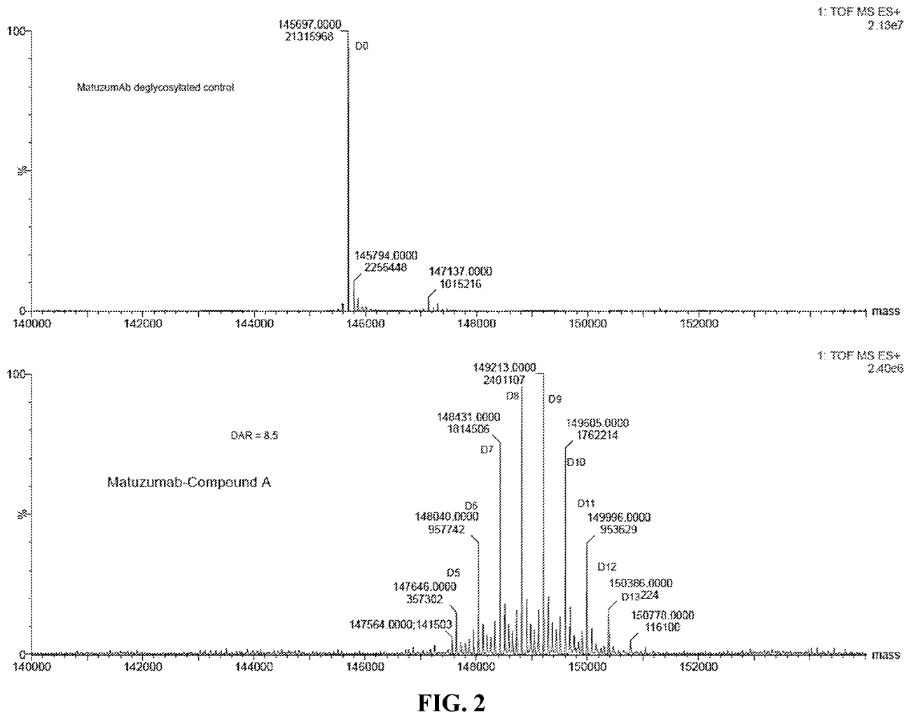

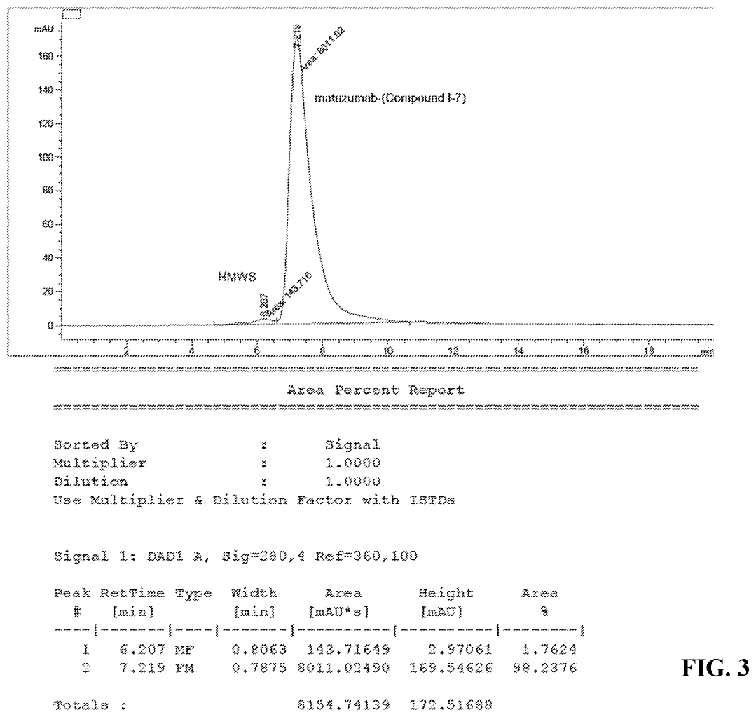

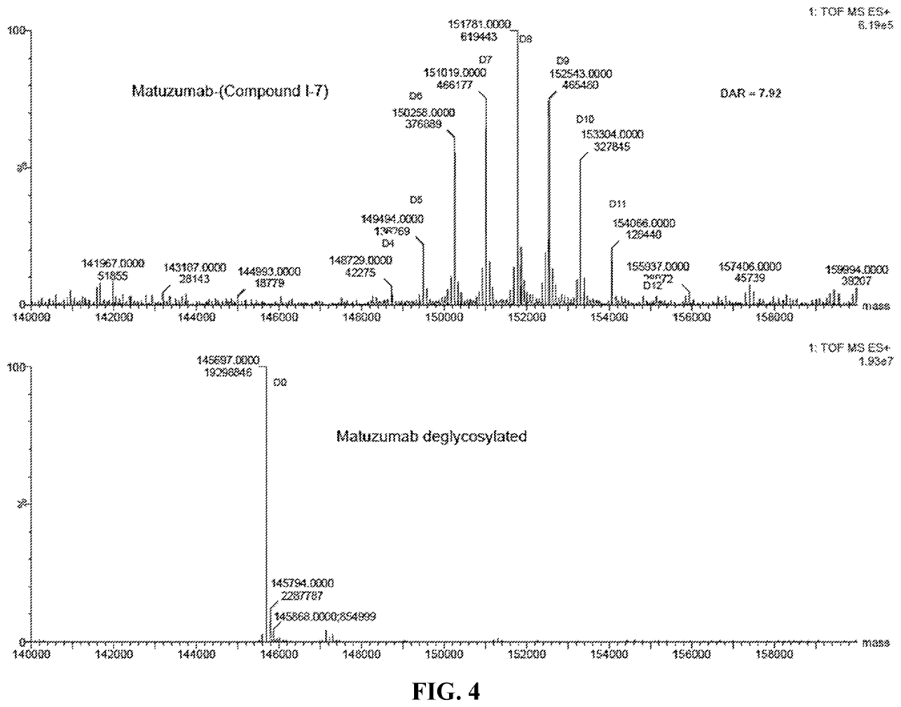

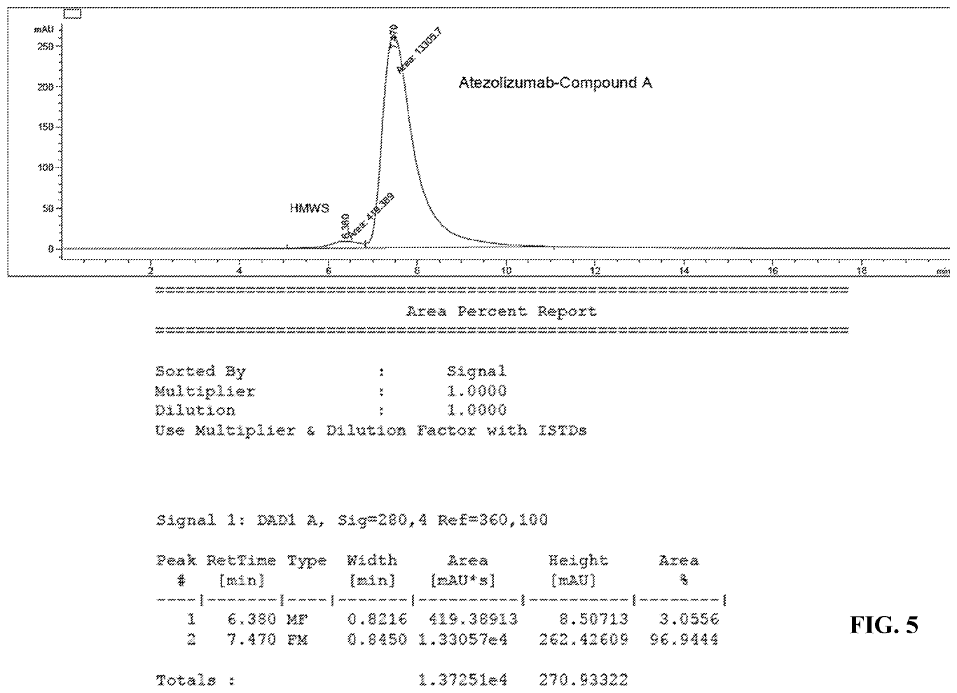

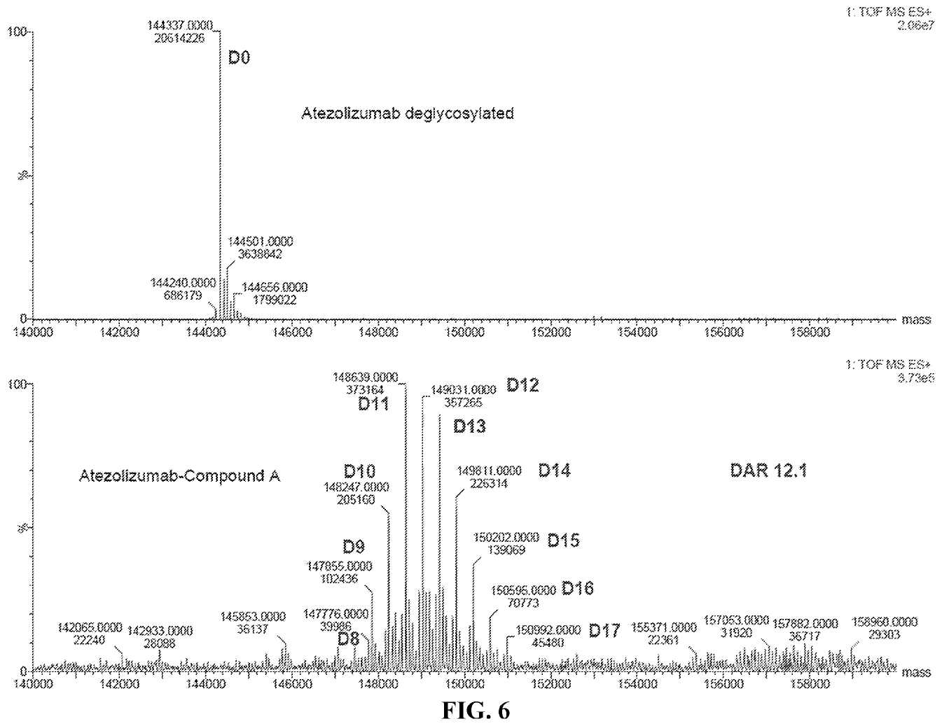

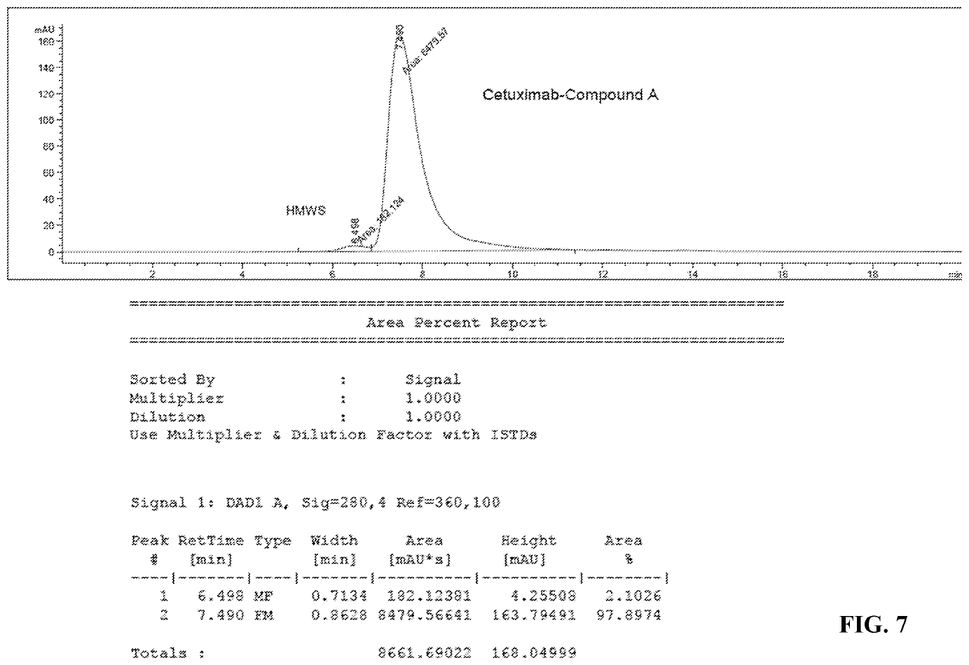

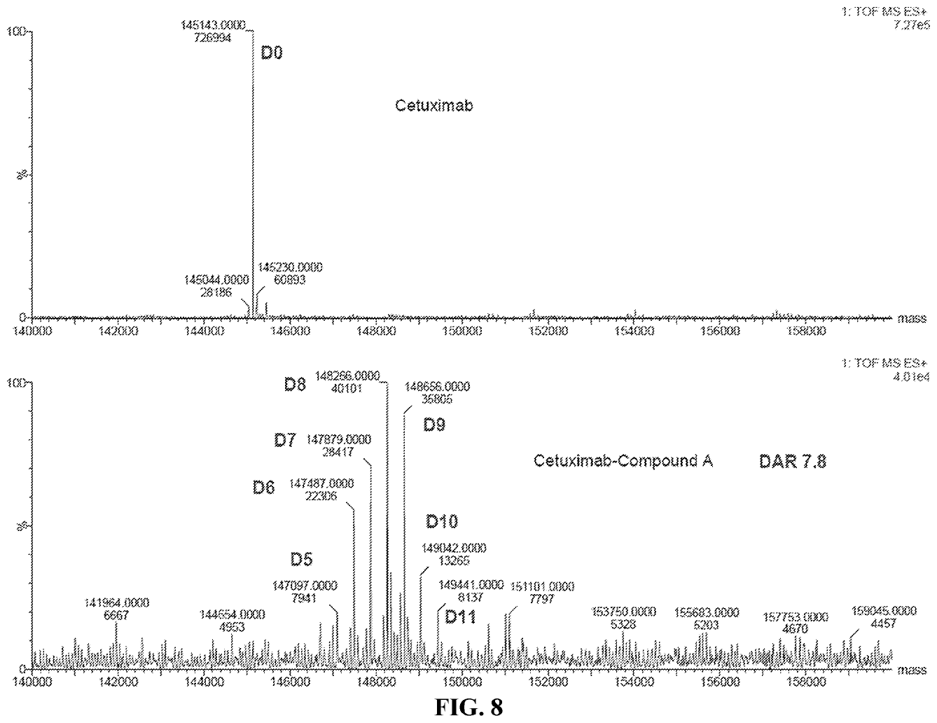

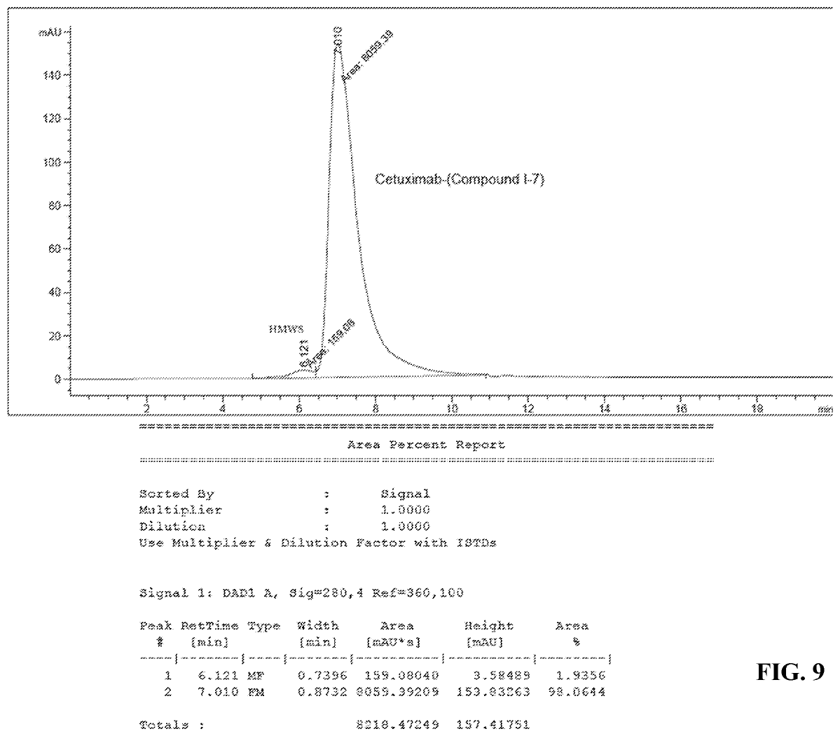

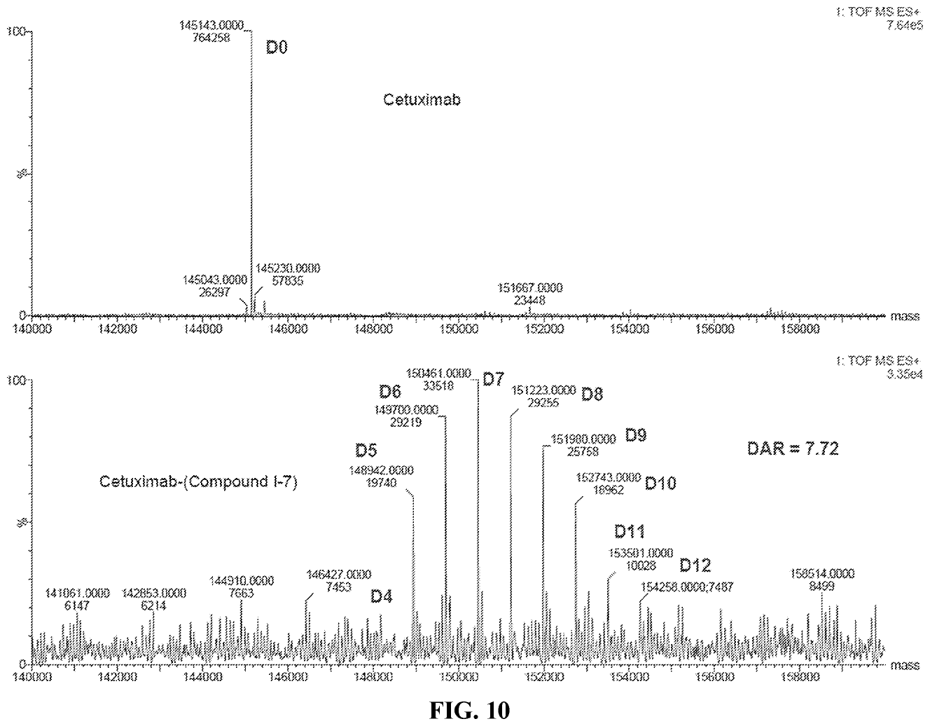

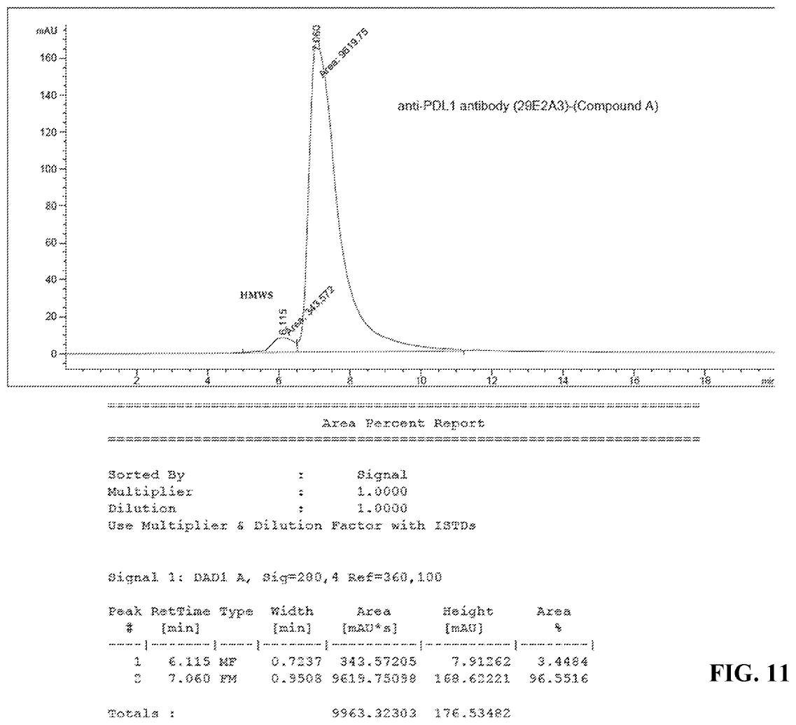

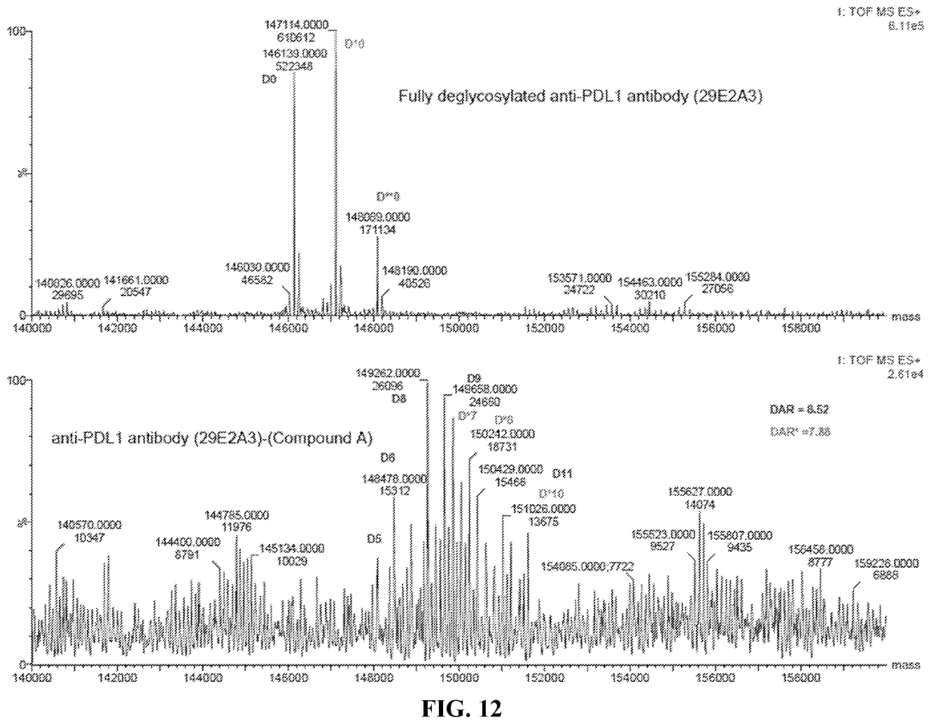

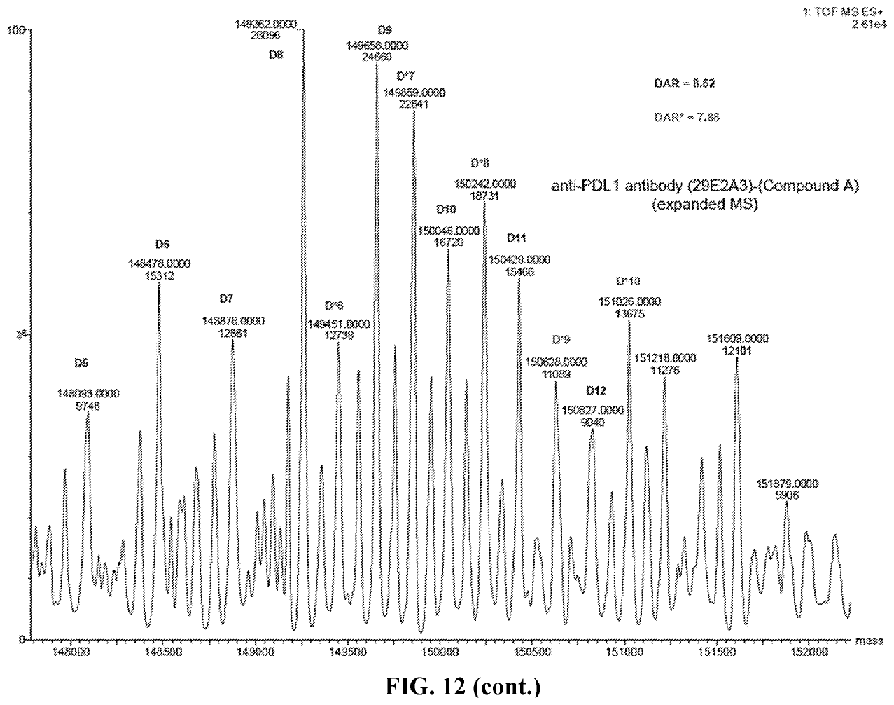

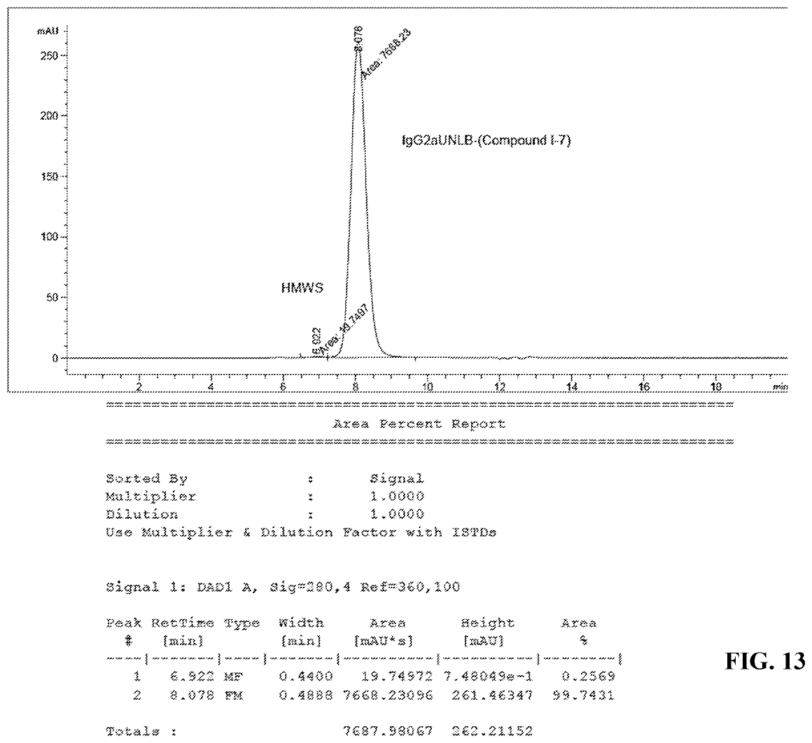

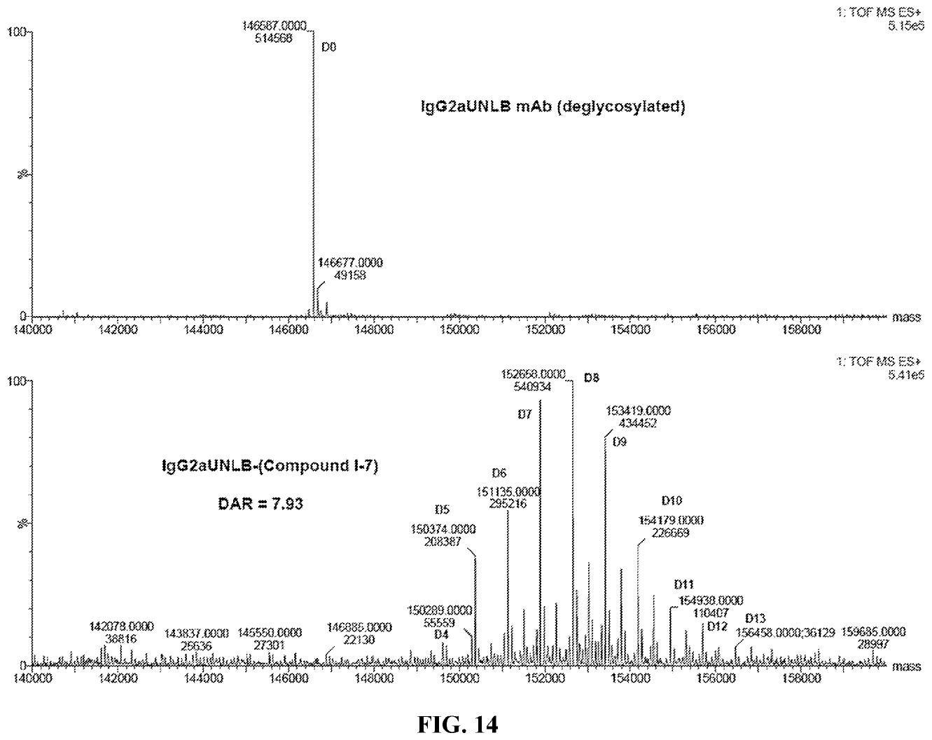

Purity of the conjugates prepared as described in Examples 137 and 138 was determined through size exclusion high performance liquid chromatography (SEC-HPLC) using a 20 minute isocratic method with a mobile phase of 0.2 M sodium phosphate, 0.2 M potassium chloride, 15 w/v isopropanol, pH 6.8. An injection volume of 10 μL was loaded to a TSKgel SuperSW3000 column, at a constant flow rate of 0.35 mL/min. Chromatographs were integrated based on elution time to calculate the purity of monomeric conjugate species. LC-MS data for the conjugates prepared as described in Examples 137 and 138 are depicted in FIG. 1-FIG. 14.

TABLE 10

| Conjugate Name | Antibody | Ligand-Linker (Compd. No.) | DAR (by MS) | Purity (by SEC) |
|---|---|---|---|---|
| Matuzumab-(Compound A) | Matuzumab | Compound A | 8.5 | >98% |
| Matuzumab-(Compound I-7) | Matuzumab | Compound I-7 | 7.92 | >98% |
| Atezolizumab-(Compound A) | Atezolizumab | Compound A | 12.1 | >96% |
| Cetuximab-(Compound A) | Cetuximab | Compound A | 7.8 | >97% |
| Cetuximab-(Compound I-7) | Cetuximab | Compound I-7 | 7.72 | >98% |
| anti-PD-L1(29E.2A3)-(Compound A) | anti-PD-L1(29E.2A3) | Compound A | 7.9-8.5 | >96% |
| IgG2a-UNLB-(Compound I-7) | IgG2a-UNLB | Compound I-7 | 7.93 | >99% |

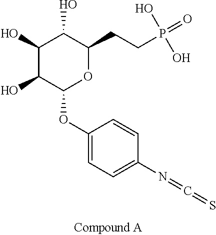 [SMILES: O=P(O)(O)CC[C@H]1O[C@H](Oc2ccc(N=C=S)cc2)[C@@H](O)[C@@H](O)[C@@H]1O]

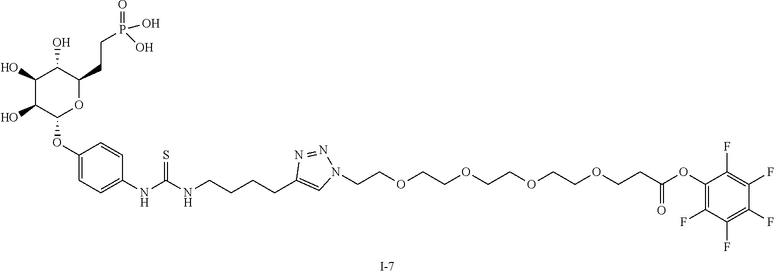

### Example 141: Antibody Disulfide Reduction and Ligand-Linker Conjugation to Antibody

This example provides an exemplary protocol for reduction of the disulfides of the antibodies described herein, and conjugation of the reduced antibodies to the ligand-linker compounds described herein.

Protocol:

Antibody Disulfide Reduction

A) Dilute antibody to 15 mg/mL (0.1 mM IgG) in PBS, pH 7.4.

B) Prepare a fresh 20 mM (5.7 mg/mL) stock solution of tris(2 carboxyethyl)phosphine (TCEP) in H₂O.

C) Add 25 μL of TCEP stock solution from step B) above to 1 mL of antibody from step A) above (0.5 mM final concentration TCEP).

D) Incubate at 37° C. for 2 hours (check for free thiols using 5,5′-dithiobis-(2-nitrobenzoic acid) (DTNB) test).

E) Aliquot the reduced antibody into 4 tubes (250 μL each).

Ligand-Linker Conjugation to Antibody

A) Prepare 10 mM stock solution of ligand-linker compound in DMSO (DMA, DMF or CH₃CN are also acceptable).

B) Add 5 equivalents of 12.5 μL stock solution from step A) above to each tube of reduced antibody (0.5 mM final concentration ligand-linker compound stock solution).

C) Incubate overnight at 4° C. for 4 hours at room temperature; check for free thiols using DTNB test.

D) Run analytical hydrophobic interaction chromatography (HIC) to determine DAR and homogeneity.

### BIOLOGICAL EXAMPLES

### Example 142: Reagents, Buffer, and Media

This example provides the reagents, buffer, and media used in the protocols described herein.

Reagents

Hela Cells (Sigma, #93021013)

Cetuximab (R&D systems)

Matuzumab (R&D systems)

Alexafluor647 labeling kit (Invitrogen)

Amicon filters, 30 kDa cut-off (Sigma Millipore)

DAPI (Invitrogen)

PFA (16% Paraformaldehyde Aqueous Solution, Electron Microscopy Sciences)

BSA (Bovine serum albumin; Sigma Millipore)

TrypLE (Invitrogen)

Accutase (Invitrogen)

Rabbit anti-EGFR (CST)

Mouse anti-β-actin (SCB)

Donkey anti-rabbit 800CW (Licor)

Donkey anti-mouse 680RD (Licor)

Odyssey Intercept Blocking Buffer (Licor)

Electroporation enhancer (IDT)

tracrRNA (IDT)

Amaxa Electroporator (Lonza)

SE Buffer (Lonza)

16-well electroporation cuvettes (Lonza)

M6P (D-Mannose-6 phosphate disodium salt hydrate; Sigma)

M6Pn (Mannose-6 phosphonate)

PBS (Phosphate buffered saline; ThermoFisher)

FACS Buffer

In 1×PBS

2% FBS (Invitrogen), 2 mM EDTA (Invitrogen), 25 mM HEPES (Invitrogen)

0.2 μM sterile filtered

Growth Media

Basal Medium: DMEM+L-Glut+Sodium Pyruvate (Invitrogen)

Additives: 10% FBS (Invitrogen), lx Anti-Anti (Invitrogen)

0.2 μM Sterile Filtered

### Example 143: CI-M6PR (IGFR2) CRISPR KO Generation

This example provides the protocol for generation of M6PR knockout (KO) cells. Cells were washed with PBS and detached using TrypLE. Media was added to the flask to deactivate trypsin. Cells were collected and counted. A total of 1×10⁶ cells was then centrifuged at 300×g for 5 minutes. The cell pellet was washed once with PBS and centrifuged at 300×g for 5 minutes. The cell pellet was resuspended in Lonza SE buffer supplemented with supplement 1 and electroporation enhancer (5 μM final). CRISPR RNP reaction began by combining equal volumes of 100 μM crRNA and tracrRNA in a PCR tube. Using a thermocycler, this mixture was heated to 95° C. for 5 minutes and allowed slowly to cool to room temperature. The annealed sgRNA product was combined with TrueCut Cas9 and allowed to incubate at RT for 15 minutes. Resuspended cells in SE buffer was mixed with the RNP reaction and allowed to incubate for 5 minutes. The entire reaction contents was then placed in a single well of a 16-well electroporation cuvette. Using a Lonza Amaxa cells were pulsed with code CA-163. After pulsing, cells were plated into a 10 cm dish. Six days post-RNP, a portion of cells were collected and lysates were prepared to test for knock-out by western.

### Example 144: Alexa Fluor 647 Conjugation

Cetuximab, matuzumab and human IgG isotype antibodies were conjugated to Alexa Fluor 647 using Alexa Fluor™ 647 Protein Labeling Kit (Invitrogen) per the manufacturer's protocol. In brief, antibodies to be labeled were diluted to 2 mg/mL in PBS to a total volume of 500 μL. A 15 DOL (degree of labeling) was used for the conjugation with the fluorophore. Free dye was removed by pre-wetting an Amicon 30 kDa filter with PBS. After incubation, the conjugation reaction was then added to the filter and spun at high speed for 10 minutes. Retained solution was then resuspended in PBS to a final volume of 1 mL and stored at 4° C. indefinitely.

### Example 145: Measurement of EGFR/IgG Levels by Surface Staining

This example provides a protocol for the measurement of the time course activity of cetuximab-(Compound A), cetuximab-(Compound I-7), matuzumab-(Compound A), and matuzumab-(Compound I-7) conjugates on surface EGFR and IgG levels in Hela parental and M6PR KO cells measured by surface staining.

Day −1

1e6 Hela parental or M6PR KO cells were plated in 2 mL of media in 6 well plates.

Day 0

Media was replaced with 1.5 mL of fresh media.

PBS, unconjugated antibodies and m6P conjugated antibodies were added to respective wells at a final concentration of 20 nM.

Day 1/2/3

Media was aspirated from wells and were washed thrice with PBS. 750 μL of Enzyme-Free Dissociation buffer was added and cells were allowed to detach on ice.

Cell were collected in tubes and spun down at 300×g for 5 mins @ 4° C.

Cells were resuspended in PBS and volume was split equally into two tubes.

All tubes were spun at 300×g for 5 mins at 4° C. One set, the PBS was aspirated and pellets were frozen at −80° C.

The other set, the PBS was aspirated and washed 2× with cold FACS buffer.

After final wash, pellets were resuspended in 300 μL FACS buffer.

The 300 μL suspension was split into three wells (100 μL each) of a 96 well plate.
Set 1: Ctx::AF647 at 1:100 dilution and incubated on ice in the dark for 1 h.
Set 2: Mtz::AF647 at 1:100 dilution and incubated on ice in the dark for 1 h.
Set 3: Goat anti-human IgG PE at 2 pg/mL and incubated on ice in the dark for 1 h.

Cells were spun down at 1000×g at 4° C. for 3 minutes and liquid was decanted. Cell pellets were resuspended in 200 μL of cold FACS buffer. Repeated 3x total.

After final wash and decant, cells were resuspended in 100 μL cold FACS buffer with DAPI (25 ug/mL final).

Stained cells were then analyzed on Biorad ZE5.

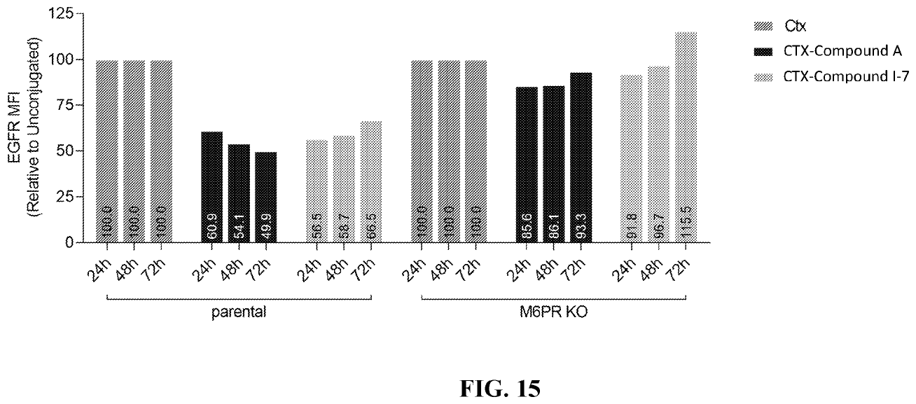

FIG. 15 shows the time course activity of cetuximab-(Compound A) and cetuximab-(Compound I-7) conjugates on surface EGFR levels in Hela parental and M6PR KO cells measured by surface staining.

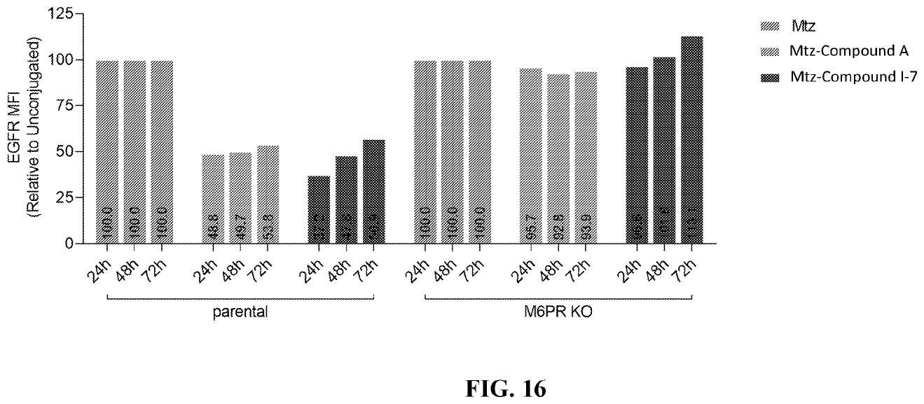

FIG. 16 shows the time course activity of matuzumab-(Compound A) and matuzumab-(Compound I-7) conjugates on surface EGFR levels in Hela parental and M6PR KO cells measured by surface staining.

These results show that the conjugates described herein can induce reduction in membrane EGFR.

### Example 146: Live-Cell EGFR Surface Staining by Flow Cytometry

This example provides an alternate protocol for the determination of the effect of matuzumab-(Compound A) or matuzumab-(Compound I-7) conjugates on surface EGFR levels measured by surface staining using flow cytometry.

Hela parental or M6PR (cation-independent mannose 6-phosphate receptor) knockout (M6PR KO) cells were plated in 6 well plates and treated with vehicle (PBS), unconjugated anti-EGFR antibody (matuzumab, Mtz), or matuzumab-(Compound A) or matuzumab-(Compound I-7) conjugates for the indicated period of time.

After incubation, media was aspirated and cells were washed three times with PBS, lifted using Accutase and pelleted by centrifugation at 300×g for 5 minutes. Cells were resuspended in cold FACS buffer and kept cold for the remainder of the staining procedure. A portion of cells were excluded from staining procedure as an unstained control. Cells were stained with either human IgG Isotype-AF647 or cetuximab-AF647 conjugates for 1 h at on ice in the dark. Cells were then spun at 300×g for 5 min at 4° C. and washed with cold FACS buffer for a total of three washes. After the final wash, cells were resuspended in 100 μL of FACS buffer with DAPI added at a final concentration of 5 pg/mL. Cells were analyzed using a BioRad ZE5 flow cytometer and data was analyzed using FlowJo software. Cells were first gated to remove debris, doublets and dead cells (DAPI negative). EGFR cell surface levels were determined based on AF647 mean fluorescence intensity (MFI).

In parental Hela cells, treatment with the M6Pn-conjugated antibodies (cetuximab-(Compound A), cetuximab-(Compound I-7), matuzumab-(Compound A), and matuzumab-(Compound I-7)) resulted in reduced cell surface levels of EGFR compared to cells treated with unconjugated antibodies (Ctx or Mtz). The reduction in cell surface EGFR was dependent on M6PR as they did not occur in M6PR knockout (M6PR KO) cells.

These results show that treatment of cells with the conjugates described herein can induce reduction in targeted cell surface receptors.

### Example 147: Measurement of Total EGFR Levels by Western Blotting

This example provides the protocol for the measurement of the time course activity of cetuximab-(Compound A), cetuximab-(Compound I-7), matuzumab-(Compound A), and matuzumab-(Compound I-7) conjugates on total EGFR levels in Hela parental and M6PR KO cells measured by traditional Western blotting.

Once all time-points from Example above were collected, all cell pellets were resuspended in 50 μL of radioimmunoprecipitation assay (RIPA) buffer (+protease/phosphatase inhibitor+nuclease).

Lysates were incubated on ice for 1 h.

Lysates were then spun at high-speed for 10 min at 4° C.

40 μL of cleared lysate was transferred to a 96 well plate.

All lysate concentrations were calculated using BCA assay (1:3 dilution).

All lysates were equalized to 2 mg/mL using RIPA as diluent.

Equal volumes (15 μL) of lysate were then mixed with LDS sample buffer (3×LDS+2.5× reducing agent).

Samples were incubated at 98° C. for mins and allowed to cool.

Samples were vortexed and spun down.

15 μL of sample was loaded onto a 26-well bis-tris 4-12% midi-gel.

Gel was allowed to run at 180V for 20 mins.

Gels were transferred to nitrocellulose membrane using iBlot 2 (20V constant, 7 mins).

Membranes were washed 1× in PBS and then placed in Odyssey blocking buffer for 1 h RT with shaking.

Primary antibodies mouse anti-β-actin (SCB) and rabbit anti-EGFR (CST) were diluted 1:1000 in blocking buffer and allowed to incubate overnight at 4° C. with shaking.

Membranes were washed thrice with PBS-T (Tween20 0.1%), at least 5 mins each wash.

Secondary antibodies anti-mouse 680rd and anti-rabbit 800cw were diluted 1:5000 in blocking buffer and allowed to incubate for 1 h at RT with shaking.

Membranes were washed thrice with PBS-T (Tween20 0.1%), at least 5 mins each wash.

Membranes were imaged using licor odyssey scanner.

### Example 142: Measurement of Total EGFR Levels by In-Cell Western Blotting

This example provides a protocol for the measurement of the dose response of cetuximab-(Compound A), cetuximab-(Compound I-7), matuzumab-(Compound A), and matuzumab-(Compound I-7) conjugates on total EGFR levels in Hela parental and M6PR KO cells measured by in-cell Western blotting.

Day −1

3e4 Hela parental or M6PR KO cells were plated 100 μL per well in a clear bottom black walled 96 well plate (Costar 3603)

Day 0

Media was decanted and 100 μL of fresh media was added back to wells.

50 μL of a 3× dose response of unconjugated and m6P conjugated antibodies were added to respective wells.

80 nM final starting concentration, 1:2 dilution. EGF was added at in 3 wells at a concentration of 50 ng/mL final.

Day 2

Media was decanted and wells were washed thrice with PBS.

Wells were fixed with 4% PFA in PBS for 15 minutes at RT.

Wells were washed thrice with PBS.

Cells were permeabilized with 0.2% triton-×100 in PBS for 15 mins. Repeated 3x total.

Cells were blocked in Odyssey blocking buffer with 0.2% triton-×100 for 1 h at RT.

Cells were stained with goat anti-EGFR (AF231, R&D, 1 pg/mL final) in block buffer overnight at 4° C.

Cells were washed 3× with PBS-T (Tween20 0.1%).

Cells were stained with donkey anti-goat 800CW secondary (1:200) and CellTag700 (1:500) in blocking buffer for 1 h at RT in dark.

Cells were washed 3× with PBS-T (Tween20 0.1%).

Last wash was decanted and plates were blotted on paper towel to remove residual liquid.

Plates were imaged on Licor scanner (3 mm offset).

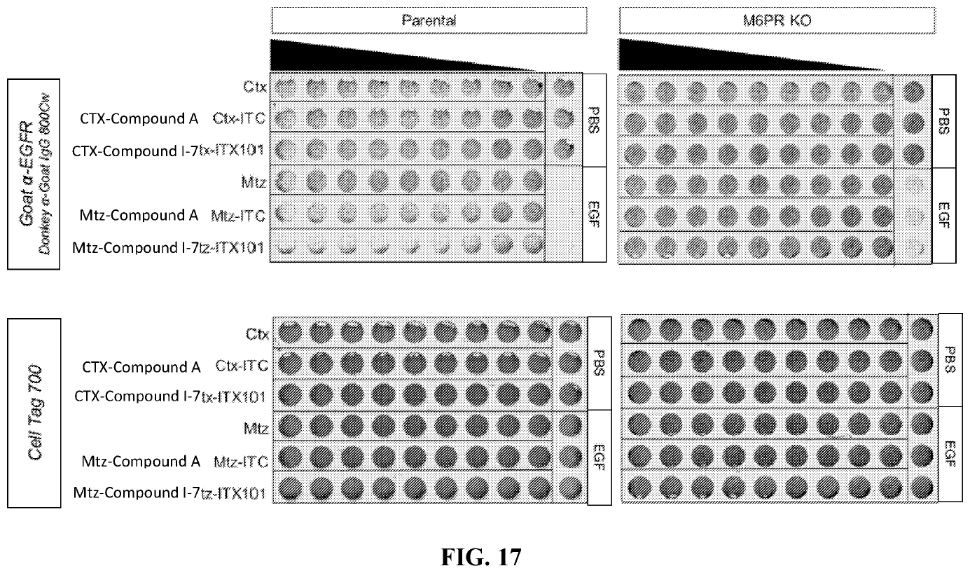

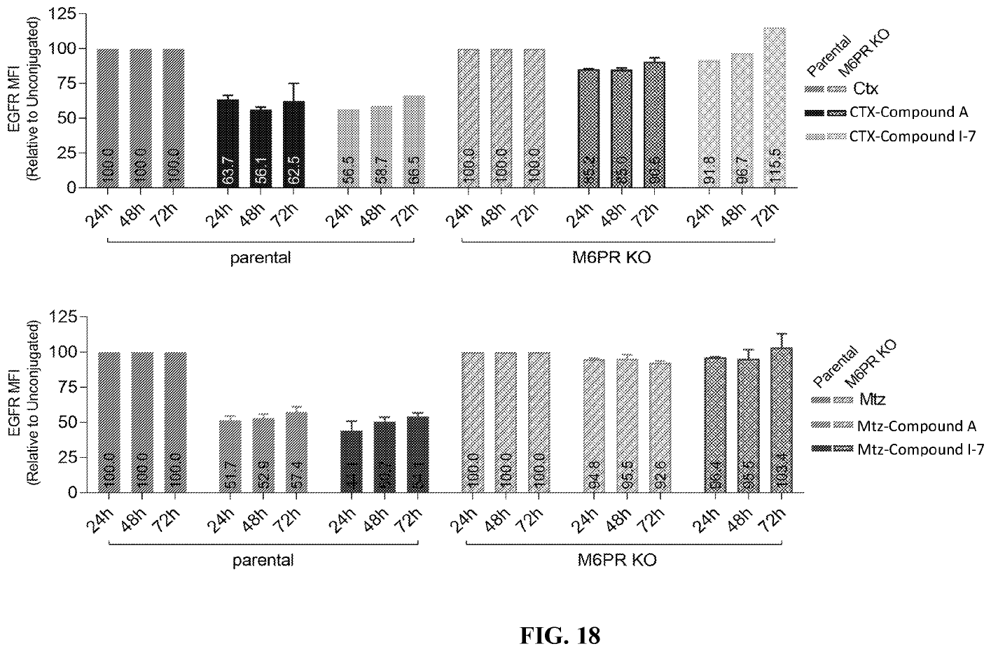

FIG. 17 shows the dose response of cetuximab-(Compound A), cetuximab-(Compound I-7), matuzumab-(Compound A), and matuzumab-(Compound I-7) conjugates on total EGFR levels in Hela parental and M6PR KO cells measured by in-cell Western blotting.

M6Pn-conjugated anti-EGFR antibodies (cetuximab-(Compound A), cetuximab-(Compound I-7), matuzumab-(Compound A), and matuzumab-(Compound I-7)) showed dose-dependent reduction in cellular EGFR compared to unconjugated antibodies alone. The reduction in EGFR was dependent on M6PR as it was observed in parental Hela cells, but not in cells lacking M6PR (M6PR KO).

These results are consistent with those of the Example above, and show that treatment of cells with the conjugates described herein can induce reduction in targeted cell surface receptors.

### Example 143: Measurement of Cellular EGFR Protein Levels Evaluated by Immunocytochemistry

This example provides an alternate protocol for the determination of the effect of M6Pn-conjugated anti-EGFR antibodies (either Mtz or Ctx) on cellular EGFR protein levels evaluated by immunocytochemistry.

HeLa parental or M6PR (cation-independent mannose 6-phosphate receptor) knockout (M6PR KO) cells were plated in 6 well plates and treated with vehicle (PBS), unconjugated anti-EGFR antibody (matuzumab, Mtz), or matuzumab-(Compound A) or matuzumab-(Compound I-7) conjugates at 37° C. for 24 hours. After incubation, media was aspirated and cells were washed thrice with PBS. Cells were fixed with 4% PFA for 10 minutes at room temperature, washed three times with PBS and then blocked with 5% BSA in PBS for 1 hour at RT. Cells were permeablizied with 0.2% Triton-X100 in PBS for 15 minutes. After washing, cells were stained with goat anti-EGFR (AF321; R&D Systems) in blocking buffer overnight at 4 C. After washing, cells were stained with anti-goat 800CW secondary or CellTag700, and imaged on Licor scanner.

M6Pn-conjugated anti-EGFR antibodies (cetuximab-(Compound A), cetuximab-(Compound I-7), matuzumab-(Compound A), and matuzumab-(Compound I-7)) showed dose-dependent reduction in cellular EGFR compared to unconjugated antibodies alone. The reduction in EGFR was dependent on M6PR as it was observed in parental Hela cells, but not in cells lacking M6PR (M6PR KO).

These results are consistent with those of Examples above, and show that treatment of cells with the conjugates described herein can induce reduction of targeted cell surface receptors.

### Example 144: Human CI-M6PR Binding Assay

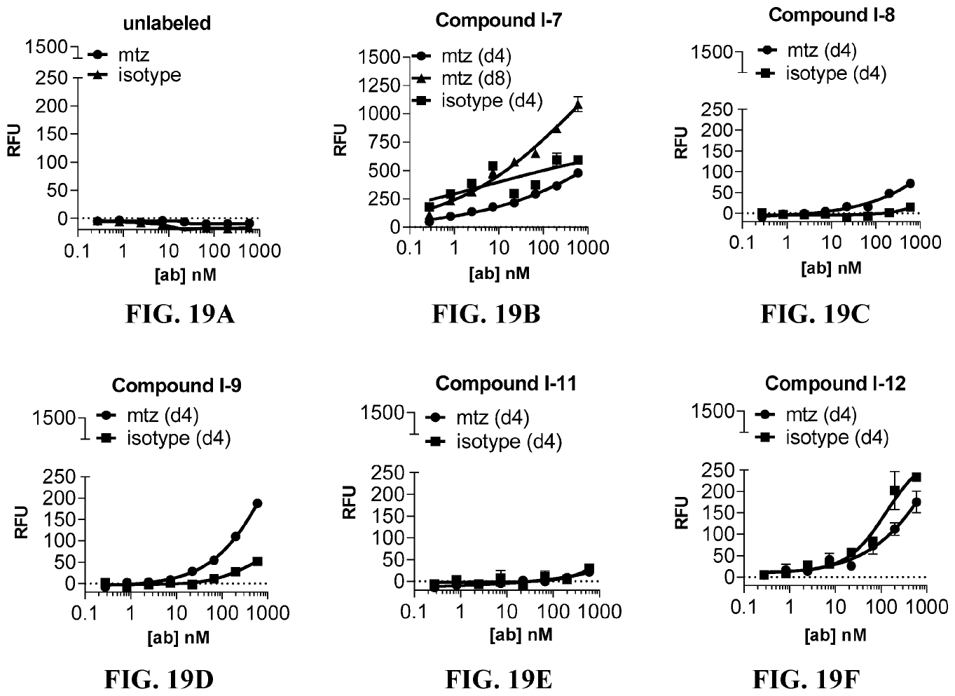

Nunc black solid bottom MaxiSorp plates were allowed to incubate overnight at 4° C. coated with 1 pg/mL of recombinant human CI-M6PR protein (R&D, 6418-GR-050) in 50 μL PBS. The next day, coating was decanted and plates were washed 3× with PBS. Wells were blocked with 350 μL of 3% BSA-PBS for 1 hour at room temperature. Blocking solution was removed and matuzumab conjugates (matuzumab-Compound I-7(d4), matuzumab-Compound I-7(d8), matuzumab-Compound I-8(d4), matuzumab-Compound I-9(d4), matuzumab-Compound I-11(d4) and matuzumab-Compound I-12(d4)) and their respective isotype controls (human IgG (bioxcell, BP0297) conjugated to the ligand-linker compounds being tested) were diluted in 3% BSA-PBS. 50 μL of diluted conjugates were added to the plate and allowed to incubate at room temperature for 2 hours. After incubation, solutions in plate were decanted and washed with 350 μL of 0.05% PBS-Tween20 three times, drying the plate each wash on a clean paper towel. 50 μL of peroxidase AffiniPure Mouse Anti-Human IgG (Jackson Immuno, 209-035-088) diluted in 3% BSA-PBS to 0.2 pg/mL was added to the plate and allowed to incubate for 1 hour at room temperature in the dark. After incubation, solutions in plate were decanted and washed with 350 μL of 0.05% PBS-Tween20 3 times, drying the plate each wash on a clean paper towel. QuantaBlu fluorogenic peroxidase substrate (ThermoFisher, 15169) was prepared per manufacturer's suggestions and equilibrated to room temperature. 50 μL of QuantaBlu solution was added to wells and allowed to incubate for 5-10 minutes at room temperature. After incubation, plates were read on a Perkin Elmer EnVision using photometric 340 and Umbelliferone 460 filter sets for excitation and emission, respectively. Data analysis and non-linear curve-fitting was performed using GraphPad Prism. FIGS. 19A-19F show various binding affinities of the conjugates tested, with Compound I-7 (d8) and Compound I-11 (d4) displaying the highest and lowest binding affinity, respectively.

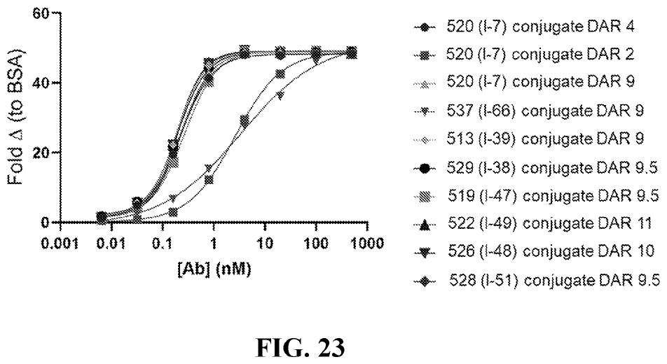

FIG. 23 shows a graph of results of a M6PR binding assay for a variety of antibody conjugates of exemplary compounds with various DAR loadings. The EC50 values of FIG. 23 are shown in Table 11. Further results from additional M6PR binding assays are shown in Table 12.

TABLE 11
EC50 values in M6PR binding assay

| Conjugate of compound # | Average Loading DAR | EC50 (nM) |
|---|---|---|
| 520 (I-7) | 4 | 0.2214 |
| 520 (I-7) | 2 | 2.603 |
| 520 (I-7) | 9 | 0.2173 |
| 537 (I-66) | 9 | 3.361 |
| 513 (I-39) | 9 | 0.1861 |
| 529 (I-38) | 9.5 | 0.1943 |
| 519 (I-47) | 9.5 | 0.2663 |
| 522 (I-49) | 11 | 0.2274 |
| 526 (I-48) | 10 | 0.1863 |
| 528 (I-51) | 9.5 | 0.1988 |

TABLE 12
EC50 values in M6PR binding assay

| Conjugate of compound # | Average Loading DAR | EC50 (nM) |
|---|---|---|
| 520 (I-7) | 4 | 0.4118 |
| 728 (I-52) | 6 | 0.2799 |
| 528 (I-51) | 2 | 4.440 |
| 528 (I-51) | 8 | 0.3009 |
| 537 (I-66) | 8 | 2.310 |
| 706 (I-41) | (maleimide-Cys conjugation) | 0.2709 |

### Example 145: Serum Pharmacokinetic Analysis for rIgG1 Antibody Conjugates of Varying Binding Affinities

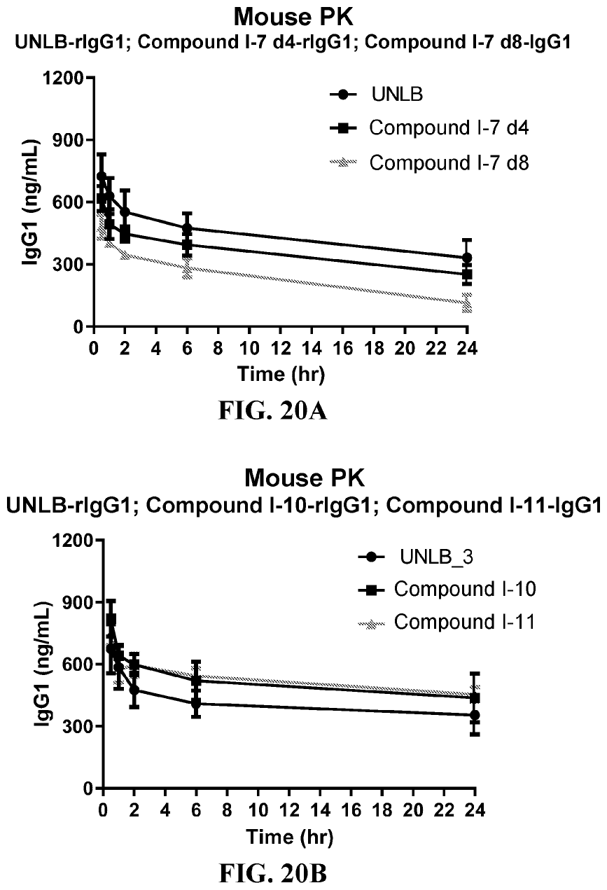

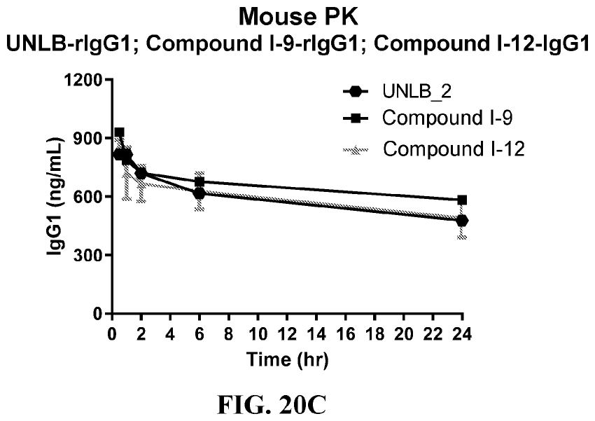

A pharmacokinetic analysis of the rIgG1 (anti-IgG2a) antibody conjugates described in the previous example was performed in mice. In particular, C57B6 mice were intravenously administered each rIgG1 antibody conjugate at 10 μg/mouse (5 mice per group). Blood was collected at 0.5, 1, 2, 6, and 24 hours and serum rIgG1 was analyzed using an ELISA kit (Abcam) according to the manufacturer's instructions. Samples were run across 3 different plates with unconjugated rIgG1 controls (UNLB-anti-IgG2a rIgG1) included on all 3 plates. FIGS. 20A-20C show the serum levels of algG2a conjugated to Compound I-7 (dar8) and (dar4) (FIG. 20A), algG2a conjugated to Compound I-10 and algG2a conjugated to Compound I-11 (FIG. 20B), and algG2a conjugated to Compound I-9 and algG2a conjugated to Compound I-12 (FIG. 20C) over time.

As shown in FIGS. 20A-20C, the results demonstrate that conjugates of ligand linkers such as Compounds I-9, I-10, I-11, and I-12 which have weaker binding affinity to M6PR compared to Compound I-7 exhibit longer half life, and therefore may be useful for tuning the pharmacokinetic properties of the conjugate.

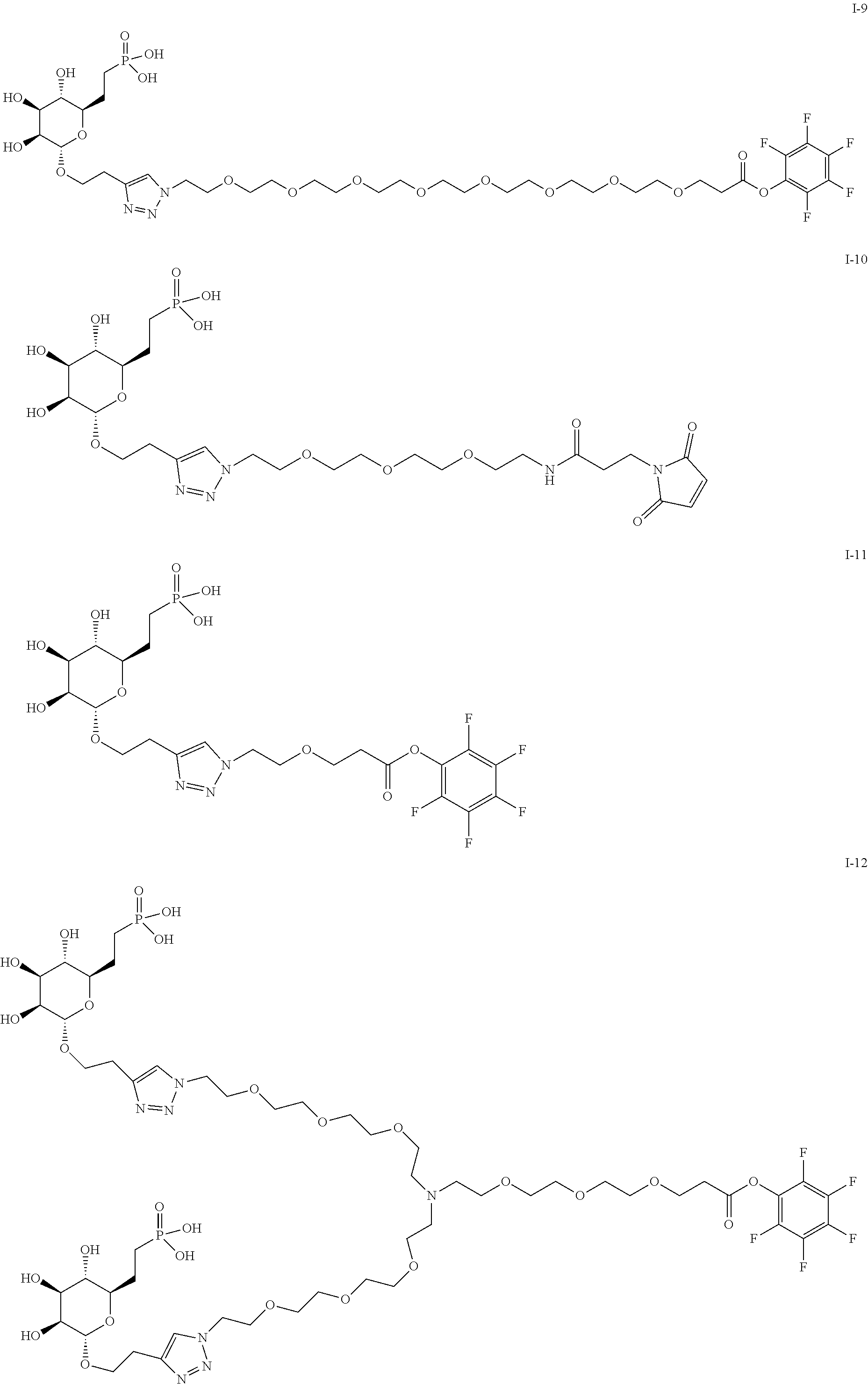 [SMILES: O=C(CCOCCOCCOCCOCCOCCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F ; O=C(CCN1C(=O)C=CC1=O)NCCOCCOCCOCCn1cc(CCO[C@H]2O[C@H](CCP(=O)(O)O)[C@@H](O)[C@H](O)[C@@H]2O)nn1 ; O=C(CCOCCn1cc(CCO[C@H]2O[C@H](CCP(=O)(O)O)[C@@H](O)[C@H](O)[C@@H]2O)nn1)Oc1c(F)c(F)c(F)c(F)c1F ; O=C(CCOCCOCCOCCN(CCOCCOCCOCCn1cc(CCO[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1)Oc1c(F)c(F)c(F)c(F)c1F]

### Example 146: Conjugates of Varying Binding Affinities Mediate Uptake of IgG2a into Cells Over Time

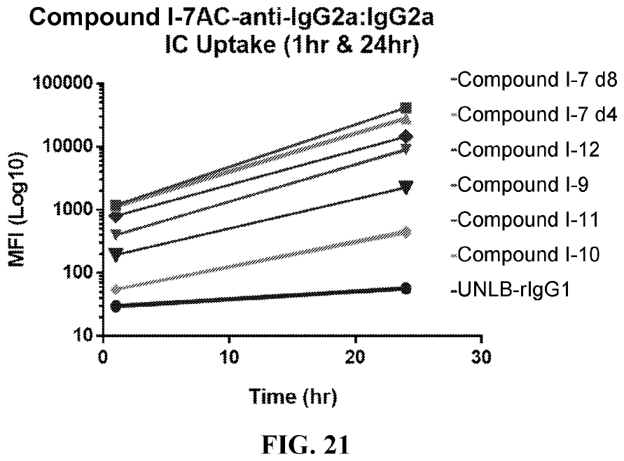

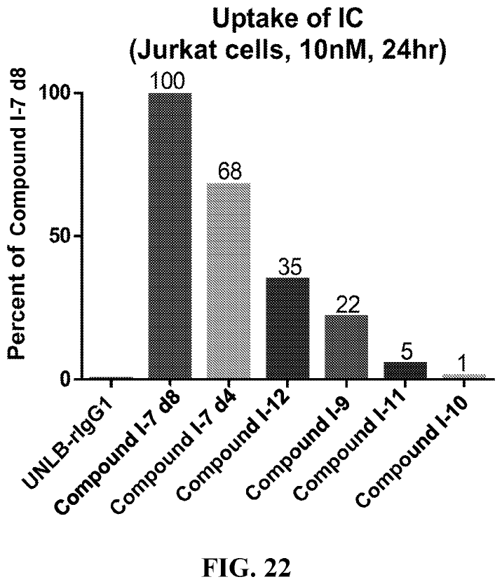

The anti-IgG2a conjugates were bound to IgG2a-Alexa488, as follows: equal molar ratios of anti-IgG2a and IgG2a-Alexa488 were added in tissue culture media for 30 minutes at room temperature. The resulting anti-IgG2a:IgG2a antibody-Alexa488 compositions were added to Jurkat cells (100 k cells/50 ul per well, n=2), and Alexa488 fluorescence levels were measured (via Alexa488 measurement) at 1 hour and 24 hours by flow cytometry. Because Alexa488 accumulates in cells, this presents a way to measure total intracellular uptake by cells over time. FIG. 21 shows the intracellular levels of algG2a conjugates Compound I-7 (dar8) and (dar4), Compound I-10, Compound I-11, Compound I-9, and Compound I-12 at 1 h and 24 h. FIG. 22 shows the intracellular uptake of the tested conjugates into Jurkat cells at 10 nM after 24 hours as a percentage of the uptake of algG2a conjugate—Compound I-7 (dar8). These data indicate that conjugates of ligand linkers with weaker binding affinity to M6PR than Compound I-7, such as Compounds I-9, I-10, I-11 and I-12, still exhibit sufficiently robust uptake and may therefore be useful for tuning the pharmacokinetic properties of the conjugate, while still capable of mediating uptake.

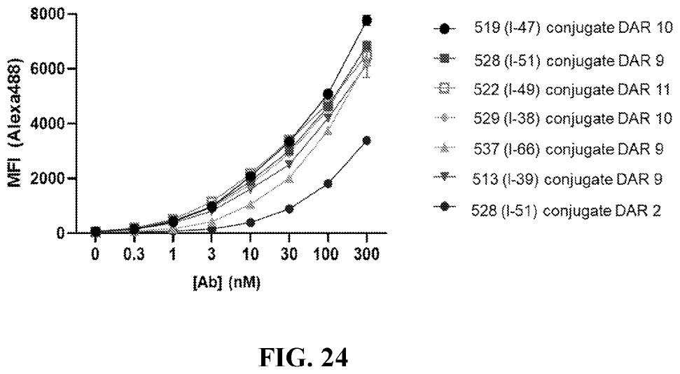

FIG. 24 shows a graph of cell fluorescence versus antibody conjugate concentration indicating that various antibody conjugates of exemplary M6PR binding compounds exhibited robust uptake into Jukat cells after one hour incubation. Conjugate compounds 519 (I-47) (DAR10), 528 (I-51) (DAR9), 522 (I-49) (DAR11), 529 (I-38) (DAR10), 537 (I-66) (DAR9), and 513 (I-39) (DAR9) all exhibited strong cell uptake. Conjugate compound 528 (I-51) with average loading DAR9 exhibited greater uptake than conjugate compound 528 (I-51) with lower average loading DAR2.

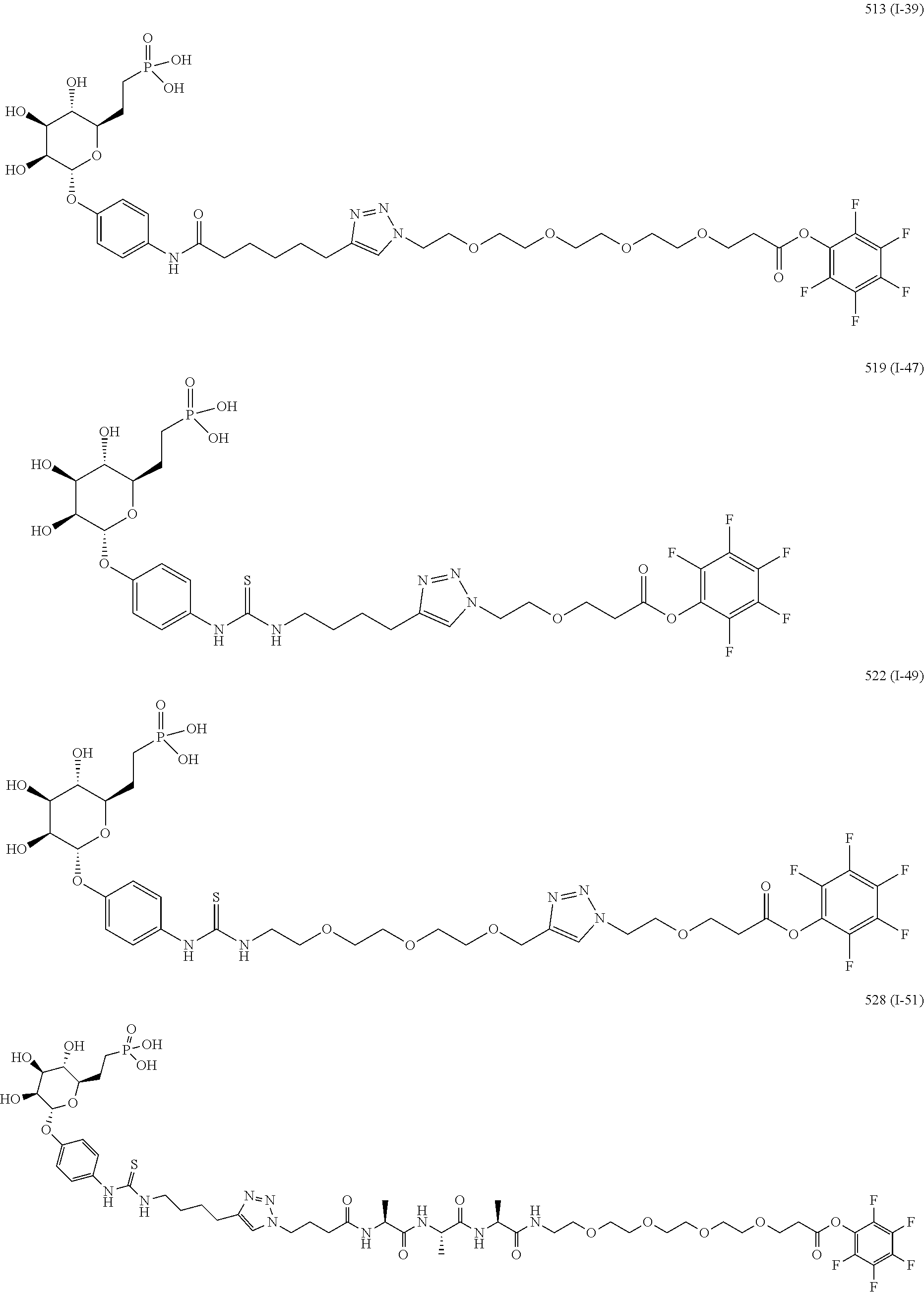

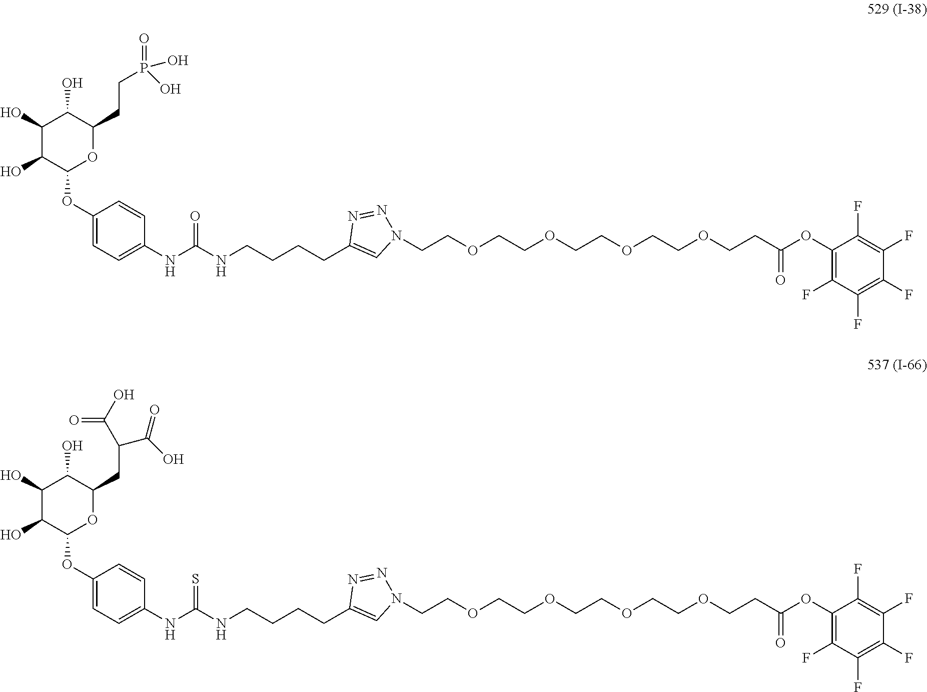 [SMILES: O=C(NCCCCc1cn(CCOCCOCCOCCOCCC(=O)Oc2c(F)c(F)c(F)c(F)c2F)nn1)Nc1ccc(O[C@H]2O[C@H](CCP(=O)(O)O)[C@@H](O)[C@H](O)[C@@H]2O)cc1 ; O=C(CCOCCOCCOCCOCCn1cc(CCCCNC(=S)Nc2ccc(O[C@H]3O[C@H](CC(C(=O)O)C(=O)O)[C@@H](O)[C@H](O)[C@@H]3O)cc2)nn1)Oc1c(F)c(F)c(F)c(F)c1F]

### Example 147: Conjugates of M6PR or ASGPR Binding Compounds Mediate Uptake of IgG2a into Human Liver Cancer Cells

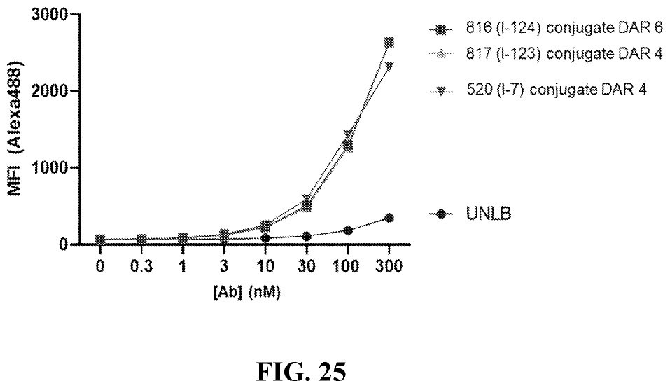

The uptake of antibody conjugates of exemplary M6PR or ASGPR binding compounds was assessed in Hep G2 cells, using a method similar to that described in Example 79. FIG. 25 shows a graph of cell fluorescence versus antibody conjugate concentration indicating that various antibody conjugates of exemplary M6PR or ASGPR binding compounds exhibited robust uptake into HepG2 cells after one hour incubation. Conjugates of compound 816 (ASGPR compound I-124) (average loading DARE), compound 817 (ASGPR compound I-123) (average loading DAR4) and compound 520 (1-7) (average loading DAR4) exhibited comparable HepG2 cellular uptake.

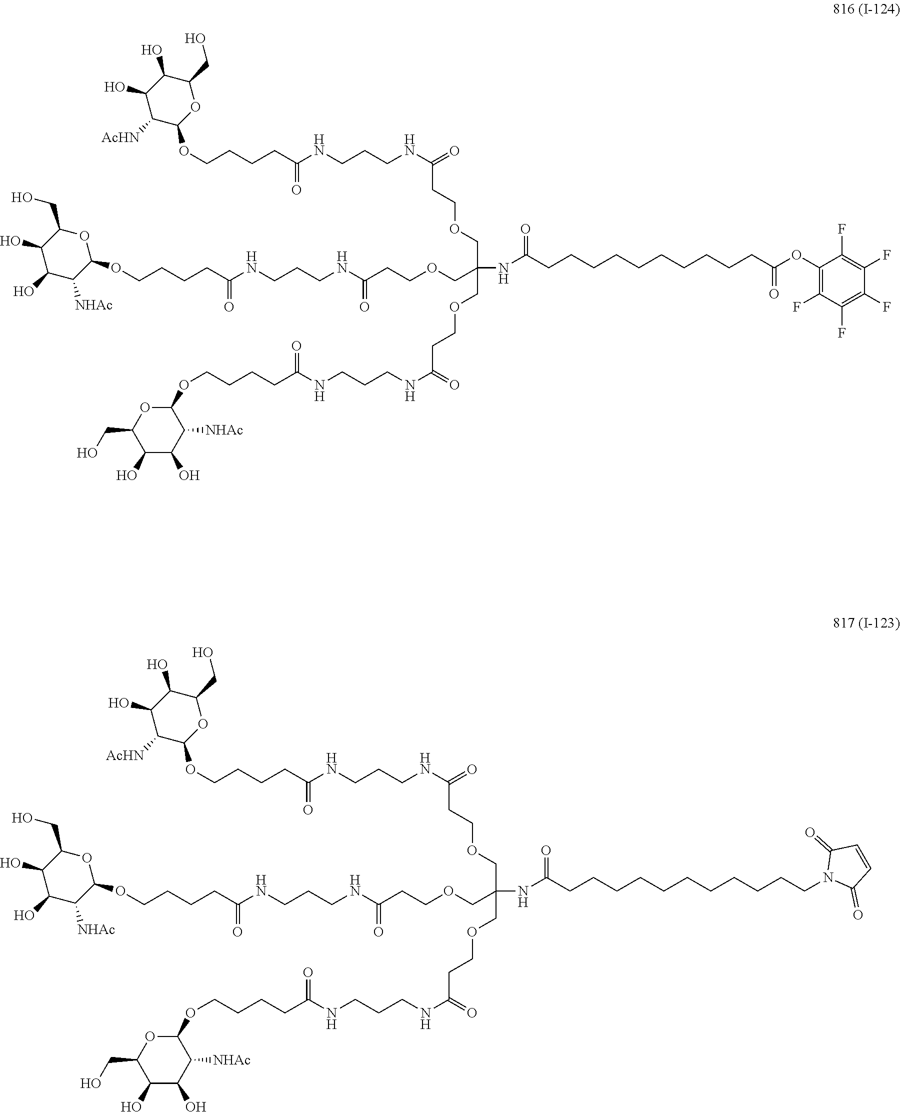

### Example 148: CI-M6PR Mediated Uptake in K562 WT or KO Cells

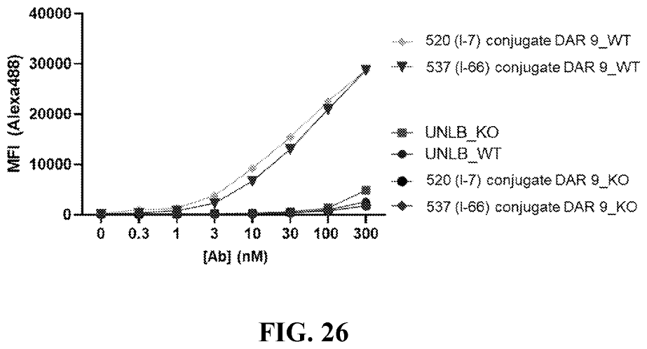

The uptake of omaluzamab antibody conjugates of exemplary compound 520 (I-7) (average loading DAR9) versus exemplary compound 537 (I-66) (average loading DAR9) was assessed in wild type (WT) K562 cells and CI-M6PR knockout (KO) cells using a similar method of that described above. FIG. 26 shows a graph of the cell uptake verus a control (UNLB) with varying concentrations of conjugate.

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A cell surface mannose-6-phosphate receptor (M6PR) binding compound of formula (XI): 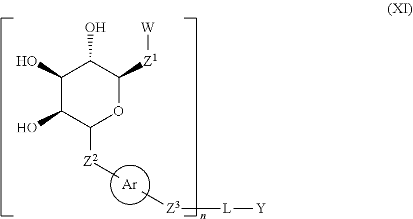
or a salt thereof,
wherein:
each W is independently a hydrophilic head group;
each Z¹ is independently selected from optionally substituted (C₁-C₃)alkylene and optionally substituted ethenylene;
each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl;
each Ar is independently an optionally substituted aryl or heteroaryl linking moiety (e.g., monocyclic or bicyclic aryl or heteroaryl, optionally substituted);
each Z³ is independently a linking moiety;
n is 1 to 500;
L is a linker; and
Y is a moiety of interest;
wherein when m is 1 and Ar is phenyl, then: i) L comprises a backbone of at least 16 consecutive atoms; ii) Y is a biomolecule; and/or ii) Z³ is amide, sulfonamide, urea or thiourea.

2. The compound of claim 1, wherein each Ar is independently selected from optionally substituted phenyl, optionally substituted pyridyl, optionally substituted biphenyl, optionally substituted naphthalene, optionally substituted triazole and optionally substituted phenylene-triazole.

3. The compound of claim 2, wherein Ar is selected from optionally substituted 1,4-phenylene, optionally substituted 1,3-phenylene, or optionally substituted 2,5-pyridylene.

4. The compound of claim 3, wherein the compound is of formula (XIIa) or (XIIb): 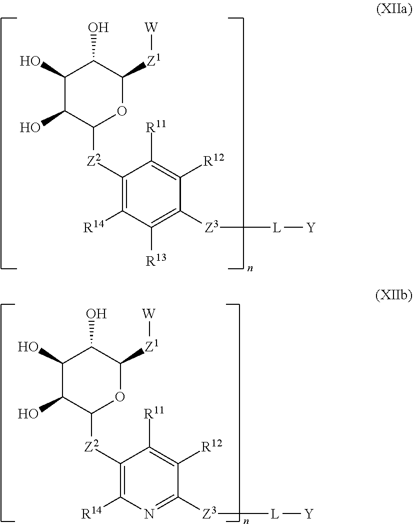
or a salt thereof,
wherein:
each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each R²⁵ is independently selected from H, and optionally substituted (C₁-C₆)alkyl.

5. The compound of claim 1, wherein Ar is an optionally substituted fused bicyclic aryl or fused bicyclic heteroaryl.

6. The compound of claim 5, wherein Ar is optionally substituted naphthalene or an optionally substituted quinoline.

7. The compound of claim 6, wherein the compound is of formula (XIIIa) or (XIIIb): 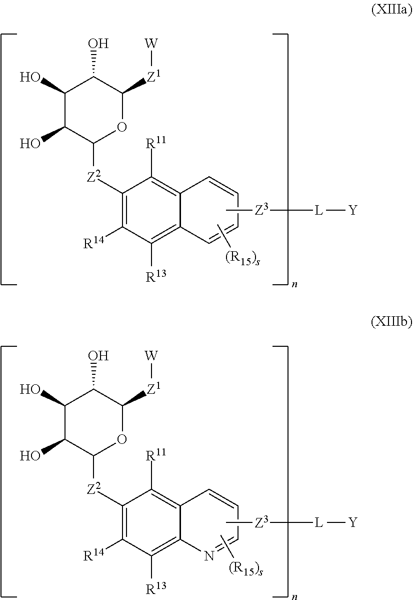
or a salt thereof,
wherein:
each R¹¹ and R¹³ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 3; and
each R²⁵ is independently selected from H, and optionally substituted (C₁-C₆)alkyl.

8. The compound of claim 7, wherein the compound is of one of formula (XIIIc) to (XIIIh): 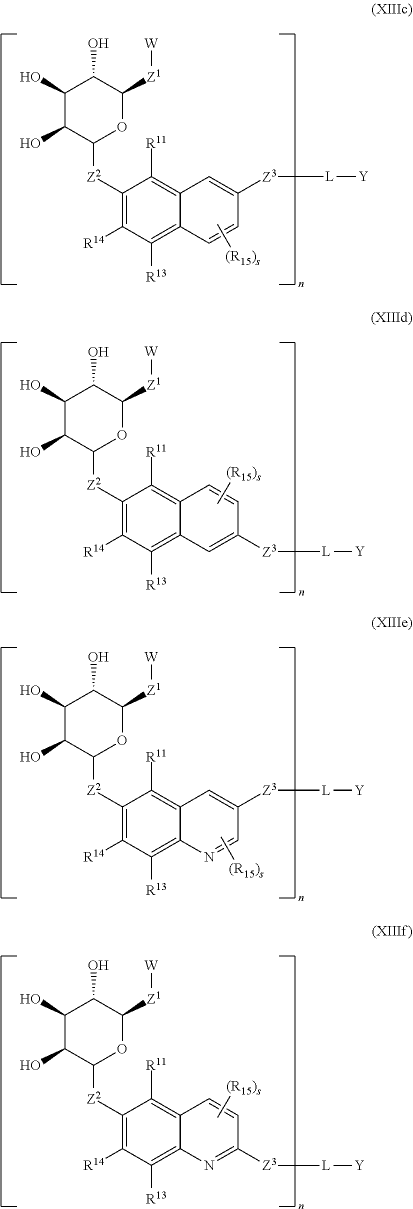 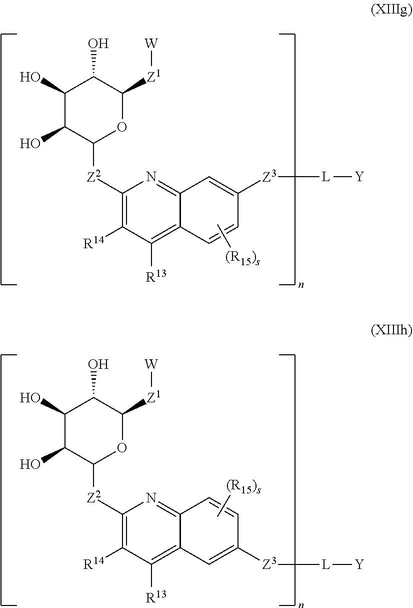
or a salt thereof.

9. The compound of claim 1, wherein Ar is optionally substituted bicyclic aryl or optionally substituted bicyclic heteroaryl and wherein the compound is of formula (XIVa) 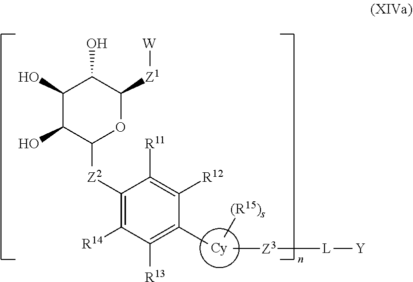
or a salt thereof,
wherein:
each Cy is independently monocyclic aryl or monocyclic heteroaryl;
each R¹¹ to R¹⁵ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵;
s is 0 to 4; and
each R²⁵ is independently selected from H, and optionally substituted (C₁-C₆)alkyl.

10. The compound of claim 9, wherein Ar is optionally substituted biphenyl, Cy is optionally substituted phenyl, and the compound is of formula (XIVb): 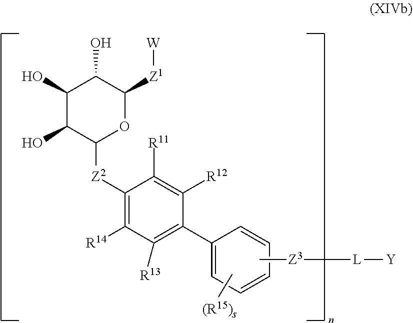
or a salt thereof.

11. The compound of claim 10, wherein the compound is of formula (XIVc) or (XIVd): 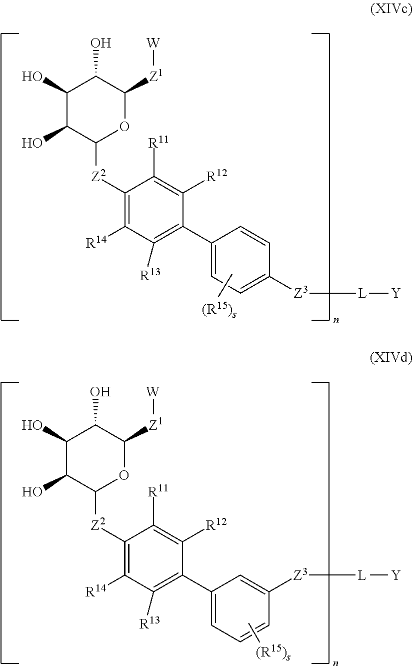
or a salt thereof.

12. The compound of any one of claims 1 to 10, wherein Ar is substituted with at least one OH substituent.

13. The compound of any one of claims 4, 6, 7, 9 and 10, wherein R¹¹ to R¹⁵ are each H.

14. The compound of any one of claims 4, 6, 7, 9 and 10, wherein at least one of R¹¹ to R¹⁵ is OH (e.g., at least two are OH).

15. The compound of any one of claims 1 to 14, wherein:
Z³ is selected from a covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X², —N(R²³)SO₂— and —SO₂N(R²³)—.
X¹ and X² are selected from O, S and NR²³; and
R²³ and R²⁴ are independently selected from H, C₍₁₋₃₎-alkyl (e.g., methyl) and substituted C₍₁₋₃₎-alkyl.

16. The compound of any one of claims 1 to 15, wherein Z³ is 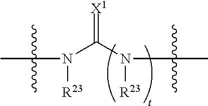
wherein:
X¹ is O or S;
t is 0 or 1; and
each R²³ is independently selected from H, C₍₁₋₃₎-alkyl (e.g., methyl) and substituted C₍₁₋₃₎-alkyl.

17. The compound of claim 16, wherein Z³ is —NHC(═X¹)NH—, wherein X¹ is O or S.

18. The compound of any one of claims 1 to 14, wherein Ar is triazole and the compound is of formula (XIIc) or (XIId):

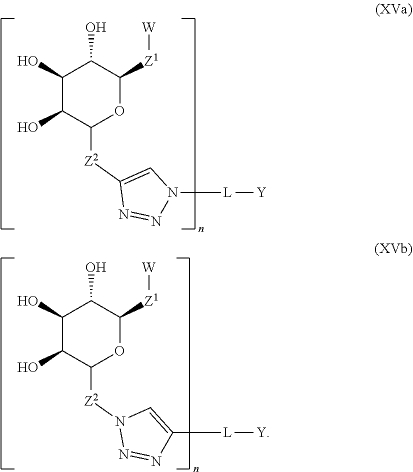

19. The compound of claim 18, wherein Z³ is optionally substituted triazole and the compound is of formula (XIIc) or (XIId): 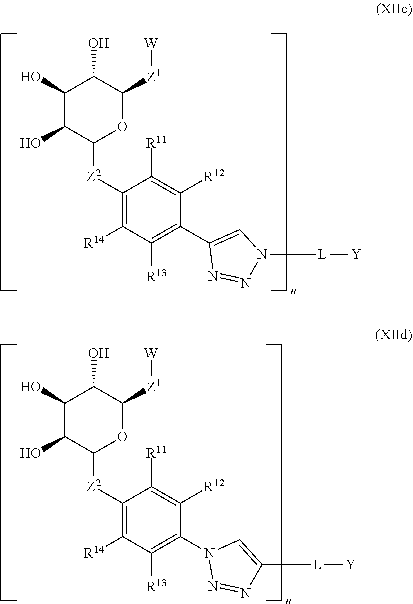
or a salt thereof,
wherein:
each R¹¹ to R¹⁴ is independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —COOR²⁵, —CONHR²⁵, and —NHCOR²⁵; and
each R²⁵ is independently selected from H, and optionally substituted (C₁-C₆)alkyl.

20. The compound of any one of claims 1 to 19, wherein —Ar—Z³— is selected from:

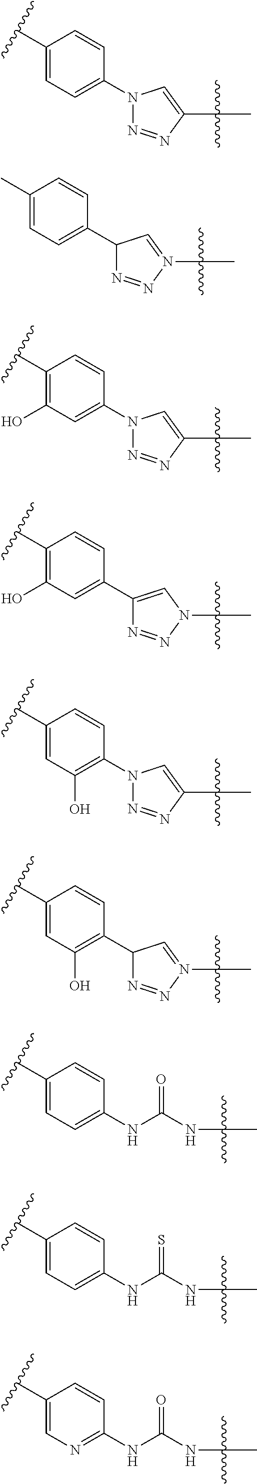

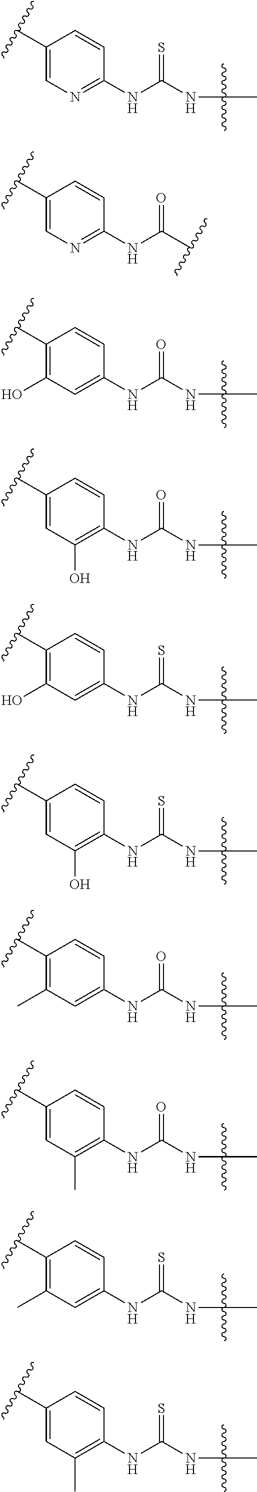

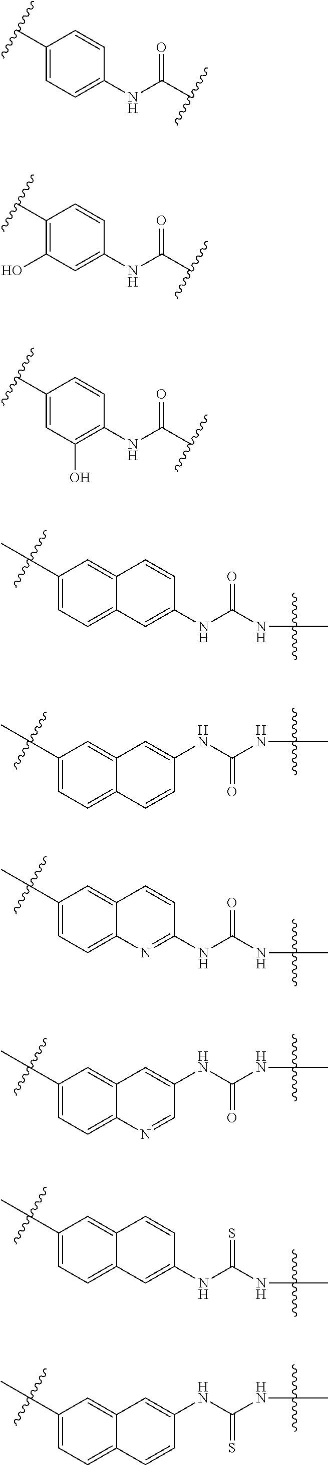

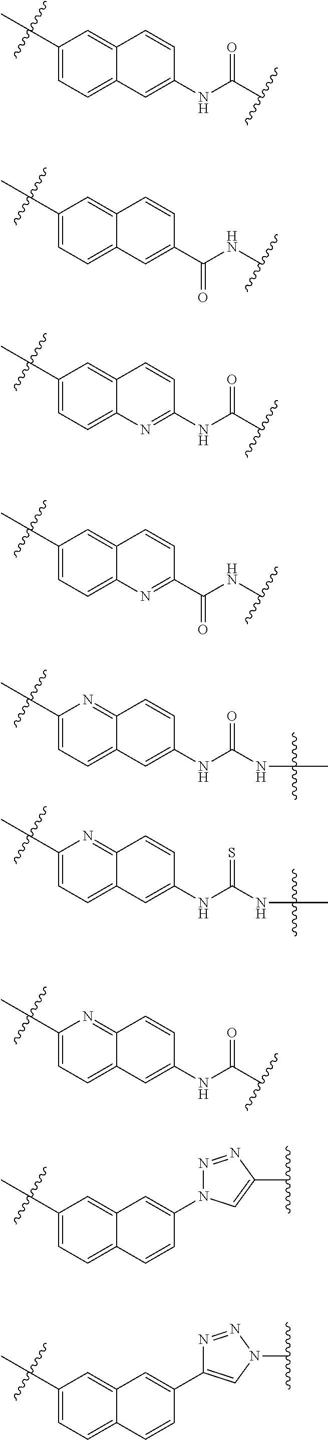

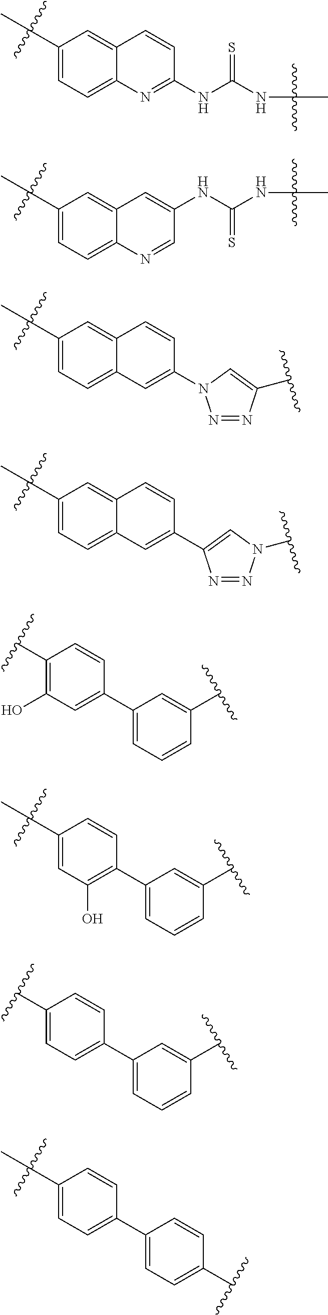

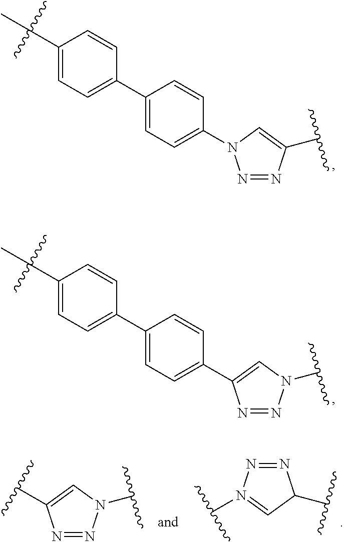

21. The compound of any one of claims 1 to 20, wherein m is at least 2, and L is a branched linker that covalently links each Ar group to Y.

22. The compound of claim 21, wherein m is 2 to 20 (e.g., m is 2 to 6, such as 2 or 3).

23. The compound of claim 21, wherein:
m is 20 to 500 (e.g., 20 to 400, 20 to 300, or 20 to 200, or 50 to 500, or 100 to 500); and
L is an α-amino acid polymer (e.g., poly-L-lysine) wherein a multitude of —Ar—Z³— groups are covalently linked to the polymer backbone via sidechain groups (e.g., via conjugation to the sidechain amino groups of lysine residues).

24. The compound of any one of claims 21 to 23, wherein m is at least 2 and each Z³ linking moiety is separated from every other Z³ linking moiety by a chain of at least 16 consecutive atoms via linker L (e.g., by a chain of at least 20, at least 25, or at least 30 consecutive atoms, and in some cases by a chain of up to 100 consecutive atoms).

25. The compound of any one of claims 1 to 24, wherein the compound is of formula (XV): 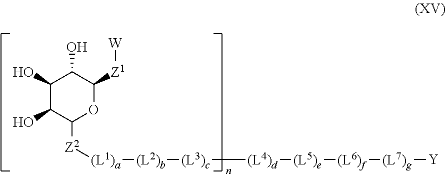
or a salt thereof,
wherein:
n is 1 to 500 (e.g., n is 1 to 20, 1 to 10, 1 to 6 or 1 to 5);
each L¹ to L⁷ is independently a linking moiety that together provide a linear or branched linker between the n Z² groups and Y, and wherein -(L¹)ₐ- comprises the linking moiety Ar that is optionally substituted aryl or heteroaryl group;
a is 1 or 2; and
b, c, d, e, f, and g are each independently 0, 1, or 2.

26. The compound of claim 25, wherein the linear or branched linker separates each Z² and Y by a chain of at least 16 consecutive atoms (e.g., at least 20 consecutive atoms, at least 30 consecutive atoms, or 16 to 100 consecutive atoms).

27. The compound of any one of claims 25 to 26, wherein n is 1 to 20.

28. The compound of any one of claims 25 to 27, wherein n is at least 2 (e.g., n is 2 or 3).

29. The compound of claim 28, wherein d is >0 and L⁴ is a branched linking moiety that is covalently linked to each L¹ linking moiety.

30. The compound of any one of claims 25 to 29, wherein the compound is of formula (XVIa) 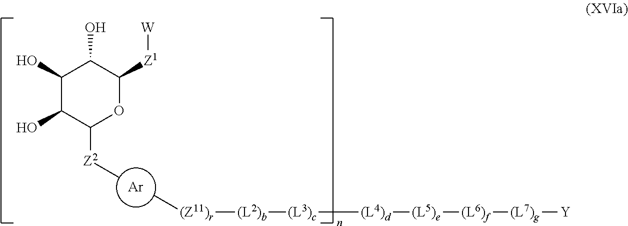
wherein:
Ar is an optionally substituted aryl or heteroaryl group (e.g., monocyclic or bicyclic or tricyclic aryl or heteroaryl group);
Z¹¹ is a linking moiety (e.g., covalent bond, heteroatom, group having a backbone of 1-3 atoms in length or triazole);
r is 0 or 1; and
n is 1 to 6.

31. The compound of claim 30, wherein Ar is selected from optionally substituted phenyl, optionally substituted pyridyl, optionally substituted biphenyl, optionally substituted naphthalene, optionally substituted quinoline, optionally substituted triazole, optionally substituted phenyl-triazole, optionally substituted biphenyl-triazole, and optionally substituted naphthalene-triazole.

32. The compound of claim 31, wherein Ar is optionally substituted 1,4-phenylene.

33. The compound of any one of claims 30 to 32, wherein Ar substituted with at least one hydroxy.

34. The compound of any one of claims 25 to 33, wherein L¹ or —Ar—(Z¹¹)ᵣ— is selected from: 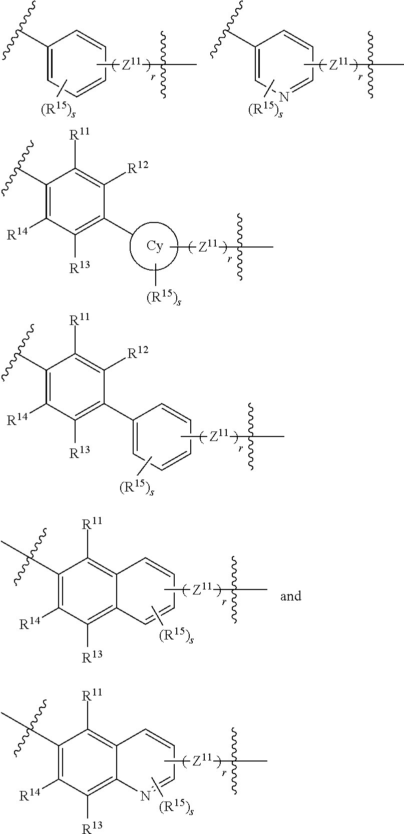
wherein:
Cy is monocyclic aryl or heteroaryl;
r is 0 or 1;
s is 0 to 4;
R¹¹ to R¹⁴ and each R¹⁵ are independently selected from H, halogen, OH, optionally substituted (C₁-C₆)alkyl, optionally substituted (C₁-C₆)alkoxy, COOH, NO₂, CN, NH₂, —N(R²⁵)₂, —OCOR²⁵, —OCOR²⁵, —CONHR²⁵, and —NHCOR²⁵, wherein each R²⁵ is independently selected from H, C₍₁₋₆₎-alkyl and substituted C₍₁₋₆₎-alkyl; and
Z¹¹ is selected from covalent bond, —O—, —NR²³—, —NR²³CO—, —CONR²³—, —NR²³CO₂—, —OCONR²³, —NR²³C(═X¹)NR²³—, —CR²⁴═N—, —CR²⁴═N—X²— and optionally substituted triazole, where X¹ and X² are selected from O, S and NR²³, wherein R²³ and R²⁴ are independently selected from H, C₍₁₋₃₎-alkyl (e.g., methyl) and substituted C₍₁₋₃₎-alkyl.

35. The compound of claim 34, wherein L¹ is

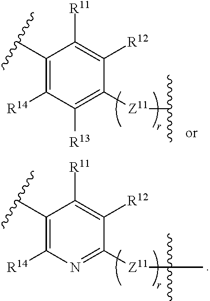

36. The compound of claim 34, wherein L¹ is

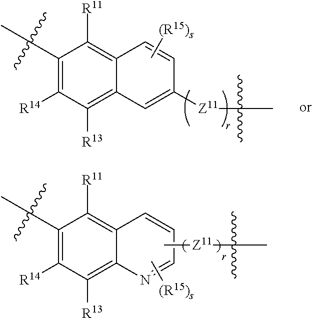

37. The compound of claim 34, wherein L¹ is selected from:

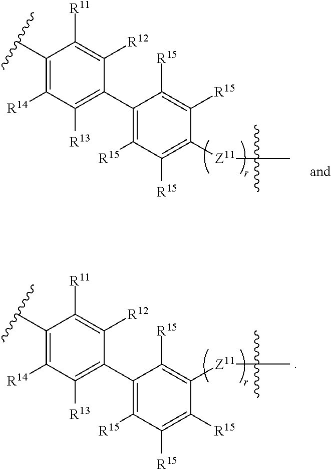

38. The compound of any one of claims 34 to 37, wherein r is 0.

39. The compound of any one of claims 34 to 37, wherein r is 1 and Z¹¹ is selected from —O—, —NR²³—, —NR²³CO—, CONR²³—, —NR²³CO₂, —OCONR²³—, —NR²³C(═X¹)NR²³—, —CR²⁴═N—, and —CR²⁴═N—X²—, wherein X¹ and X² are selected from O, S and NR²³, and each R²³ and R²⁴ is independently selected from H, C₍₁₋₃₎-alkyl (e.g., methyl) and substituted C₍₁₋₃₎-alkyl.

40. The compound of any one of claims 34 to 37, wherein r is 1 and Z¹¹ is 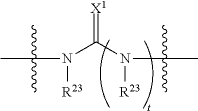
wherein:
X¹ is O or S;
t is 0 or 1; and
each R²³ is independently selected from H, C₍₁₋₃₎-alkyl (e.g., methyl) and substituted C₍₁₋₃₎-alkyl.

41. The compound of claim 40, wherein Z¹¹ is —NHC(═X¹)NH—, wherein X¹ is O or S.

42. The compound of any one of claims 34 to 37, wherein r is 1 and Z¹¹ is triazole.

43. The compound of any one of claims 1 to 42, wherein Y is selected from small molecule, dye, fluorophore, monosaccharide, disaccharide, trisaccharide, and chemoselective ligation group or precursor thereof.

44. The compound of any one of claims 1 to 42, wherein Y is a biomolecule.

45. The compound of claim 44, wherein the biomolecule is selected from peptide, protein, polynucleotide, polysaccharide, glycoprotein, lipid, enzyme, antibody, and antibody fragment.

46. The compound of any one of claims 1 to 45, wherein Y is a moiety that specifically binds a target protein.

47. The compound of claim 46, wherein the target protein is a membrane bound protein.

48. The compound of claim 46, wherein the target protein is an extracellular protein.

49. The compound of any one of claims 46 to 49, wherein Y is selected from antibody, antibody fragment (e.g., antigen-binding fragment of an antibody), chimeric fusion protein, an engineered protein domain, D-protein binder of target protein, aptamer, peptide, enzyme substrate and small molecule inhibitor or ligand.

50. The compound of claim 49, wherein Y is antibody or antibody fragment that specifically binds the target protein and the compound is of formula (Va): 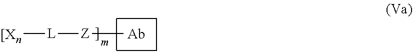
or a pharmaceutically acceptable salt thereof,
wherein:
n is 1 to 20;
m is an average loading of 1 to 80;
Ab is the antibody or antibody fragment that specifically binds the target protein; and
Z is a residual moiety resulting from the covalent linkage of a chemoselective ligation group to a compatible group of Ab.

51. The compound of claim 49, wherein Y is a small molecule inhibitor or ligand of the target protein.

52. The compound of any one of claims 1 to 51, wherein the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³, —SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H, 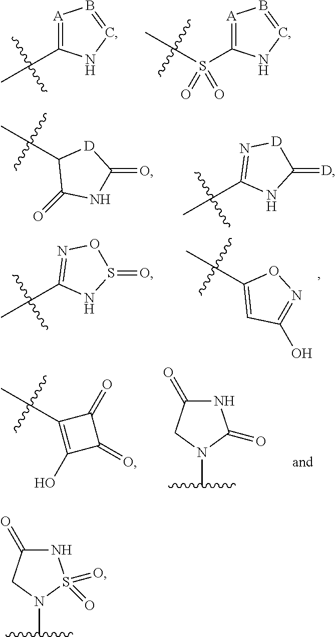
or a salt thereof,
wherein:
R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
A, B, and C are each independently CH or N; and
D is each independently O or S.

53. The compound of claim 52, wherein W is selected from —P═O(OH)₂, —SO₃H, —COOH and —CH(COOH)₂, or a salt thereof.

54. The compound of any one of claims 1 to 53, wherein:
Z′ is —(CH₂)ⱼ— or —(C(R²²)₂)ⱼ—, wherein each R²² is independently selected from H, halogen (e.g., F) and optionally substituted (C₁-C₆)alkyl; and
j is 1 to 3.

55. The compound of any one of claims 1 to 53, wherein Z¹ is —CH═CH—.

56. The compound of any one of claims 1 to 55, wherein Z² is O or S.

57. The compound of any one of claims 1 to 55, wherein Z² is —NR²¹—.

58. The compound of any one of claims 1 to 55, wherein Z² is —C(R²²)₂—, wherein each R²² is independently selected from H, halogen (e.g., F) and optionally substituted (C₁-C₆)alkyl.

59. The compound of any one of claims 1 to 53, wherein:
Z¹ is selected from —(CH₂)ⱼ—, substituted (C₁-C₃)alkylene and —CH═CH—;
j is 1 to 3; and
Z² is selected from O and CH₂.

60. The compound of claim 60, wherein:
Z¹ is —(CH₂)₂—, —CH₂—CF₂— or —CH₂—CHF—; and
Z² is O.

61. The compound of claim 60, wherein:
Z¹ is —(CH₂)₂—, —CH₂—CF₂— or —CH₂—CHF—; and
Z² is CH₂.

62. The compound of claim 60, wherein:
Z¹ is —CH═CH—; and
Z² is O.

63. The compound of claim 60, wherein:
Z¹ is —CH═CH—; and
Z² is CH₂.

64. The compound of any one of claims 1 to 63, wherein X is selected from:

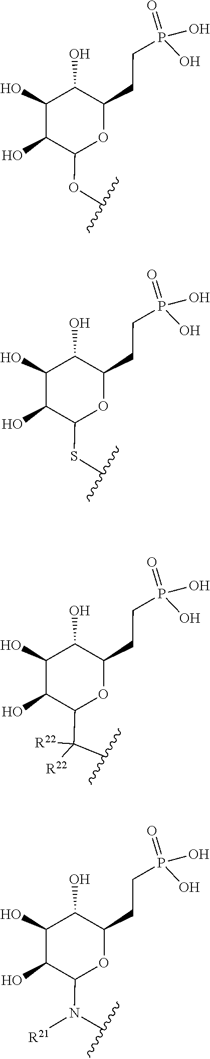

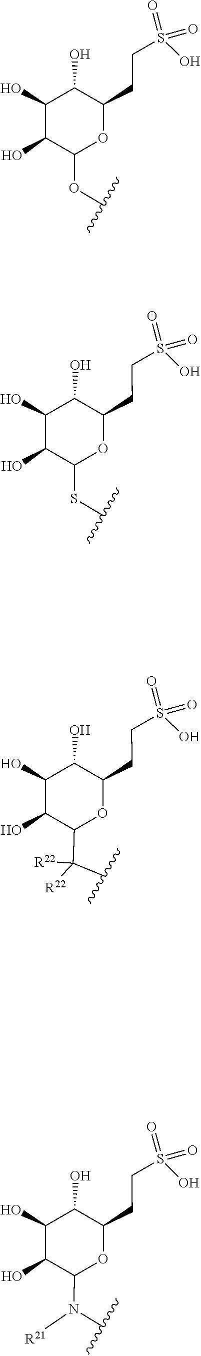

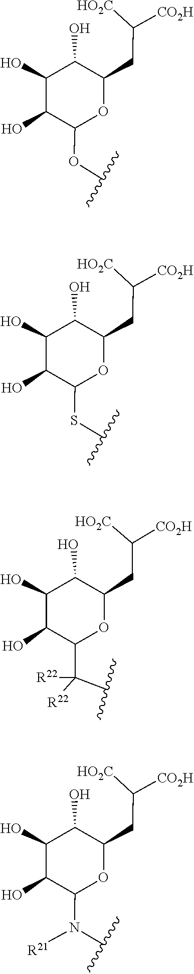

65. The compound of any one of claims 25 to 64, wherein n is 1 to 6 (e.g., n is 1 to 5, or 2 to 6, or 1, 2 or 3), and wherein:
when d is 0, n is 1;
when d is 1, n is 1 to 3; and
when d is 2, n is 1 to 6.

66. The compound of any one of claims 25 to 65, wherein:
each L² is independently selected from —C₁₋₆-alkylene-, —NHCO—C₁₋₆-alkylene-, —CONH—C₁₋₆-alkylene-, —O(CH₂)ₚ—, and —(OCH₂CH₂)ₚ—, wherein p is 1 to 10; and
each L³ is independently selected from: 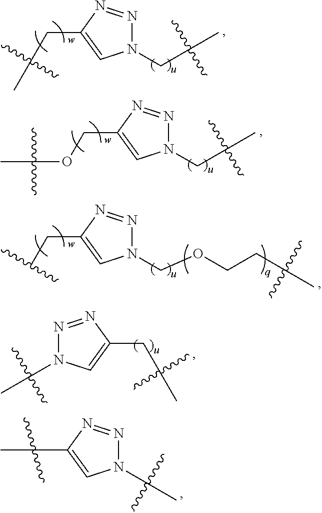
and —(OCH₂CH₂)_{q}—, wherein q is 1 to 10, u is 0 to 10, and w is 1 to 10.

67. The compound of any one of claims 25 to 66, wherein when n is 2 or more, at least one L⁴ is present and is a branched linking moiety.

68. The compound of any one of claims 25 to 67, wherein each L⁴ is independently selected from:
—OCH₂CH₂—, 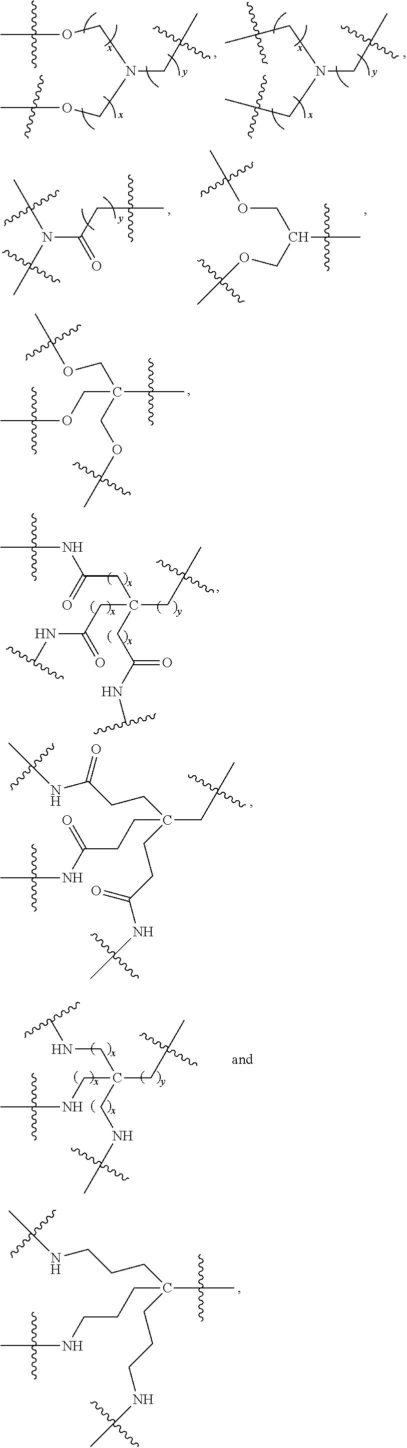
wherein each x and y are each independently 1 to 10.

69. The compound of any one of claims 25 to 68, wherein:
each L⁵ is independently —NHCO—C₁₋₆-alkylene-, —CONH—C₁₋₆-alkylene-, —C₁₋₆-alkylene-, 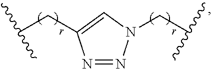
or —(OCH₂CH₂)ᵣ—;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—; and
r, s, and t are each independently 1 to 20.

70. The compound of any one of claims 25 to 69, wherein a is 1.

71. The compound of any one of claims 25 to 70, wherein at least one of b, c, e, f, and g is not 0.

72. The compound of any one of claims 25 to 71, wherein at least one of b or c is not 0 and at least one of e, f, and g is not 0.

73. The compound of any one of claims 25 to 72, wherein a, b, and c are each independently 1 or 2.

74. The compound of any one of claims 1 to 73, wherein the linker L is selected from any one of the structures of Tables 2-3.

75. The compound of any one of claims 1 to 74, wherein the compound is selected from the compounds of Tables 5-9.

76. A cell surface receptor binding conjugate of formula (I):
Xₙ-L-Y (I)
or a salt thereof,
wherein:
X is a moiety that binds to a cell surface asialoglycoprotein receptor (ASGPR) or a moiety that binds to a cell surface mannose-6-phosphate receptor (M6PR);
n is 1 to 500 (e.g., n is 1 to 20, 1 to 10, 1 to 6 or 1 to 5); and
L is a linker;
Y is a biomolecule that specifically binds a target protein.

77. The conjugate of claim 76, wherein the conjugate is formula (V): 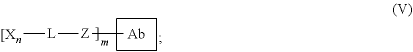
or a pharmaceutically acceptable salt thereof,
wherein:
n is 1 to 20;
m is an average loading of 1 to 80;
Ab is an antibody or antibody fragment that specifically binds the target protein; and
Z is a residual moiety resulting from the covalent linkage of a chemoselective ligation group to a compatible group of Ab.

78. The conjugate of claim 76 or 77, wherein n is 1 to 6.

79. The conjugate of claim 76 or 77, wherein n is 2 or less.

80. The conjugate of claim 79, wherein n is 1.

81. The conjugate of claim 76 or 77, wherein n is at least 2.

82. The conjugate of claim 81, wherein n is 2.

83. The conjugate of claim 81, wherein n is 3.

84. The conjugate of claim 81, wherein n is 4.

85. The conjugate of any one of claims 76 to 84, wherein m is 1 to 20.

86. The conjugate of any one of claims 76 to 84, wherein m is 1 to 12.

87. The conjugate of any one of claims 76 to 86, wherein m is at least about 2.

88. The conjugate of any one of claims 76 to 86, wherein m is at least about 3.

89. The conjugate of any one of claims 76 to 86, wherein m is at least about 4.

90. The conjugate of any one of claims 77 to 89, wherein Z is a residual moiety resulting from the covalent linkage of a thiol-reactive chemoselective ligation group to one or more cysteine residue(s) of Ab.

91. The conjugate of any one of claims 76 to 89, wherein Z is a residual moiety resulting from the covalent linkage of an amine-reactive chemoselective ligation group to one or more lysine residue(s) of Ab.

92. The conjugate of any one of claims 76 to 91, wherein X is a moiety that binds M6PR and is of the formula: 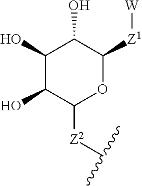
or a salt thereof,
wherein:
each W is independently a hydrophilic head group;
each Z¹ is independently selected from optionally substituted (C₁-C₃)alkylene and optionally substituted ethenylene; and
each Z² is independently selected from O, S, NR²¹ and C(R²²)₂, wherein each R²¹ is independently selected from H, and optionally substituted (C₁-C₆)alkyl, and each R²² is independently selected from H, halogen (e.g., F) and optionally substituted (C₁-C₆)alkyl.

93. The conjugate of claim 92, wherein the hydrophilic head group W is selected from —OH, —CR²R²OH, —OP═O(OH)₂, —SP═O(OH)₂, —NR³P═O(OH)₂, —OP═O(SH)(OH), —SP═O(SH)(OH), —OP═S(OH)₂, —OP═O(N(R³)₂)(OH), —OP═O(R³)(OH), —P═O(OH)₂, —P═S(OH)₂, —P═O(SH)(OH), —P═S(SH)(OH), P(═O)R¹OH, —PH(═O)OH, —(CR²R²)—P═O(OH)₂, —SO₂OH (i.e., —SO₃H), —S(O)OH, —OSO₂OH, —COOH, —CN, —CONH₂, —CONHR³, —CONR³R⁴, —CONH(OH), —CONH(OR³), —CONHSO₂R³, —CONHSO₂NR³R⁴, —CH(COOH)₂, —CR¹R²COOH, —SO₂R³,—SOR³R⁴, —SO₂NH₂, —SO₂NHR³, —SO₂NR³R⁴, —SO₂NHCOR³, —NHCOR³, —NHC(O)CO₂H, —NHSO₂NHR³, —NHC(O)NHS(O)₂R³, —NHSO₂R³, —NHSO₃H, 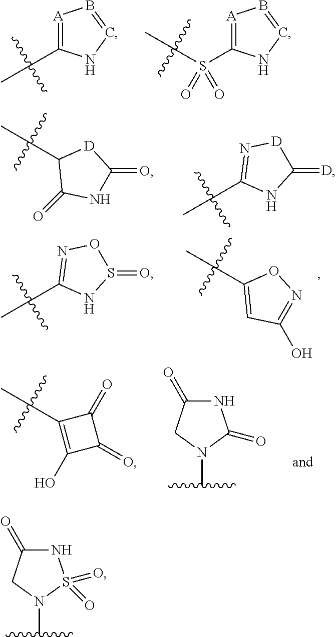
or a salt thereof,
wherein:
R¹ and R² are independently hydrogen, SR³, halo, or CN, and R³ and R⁴ are independently H, C₁₋₆ alkyl or substituted C₁₋₆ alkyl (e.g., —CF₃ or —CH₂CF₃);
A, B, and C are each independently CH or N; and
D is each independently O or S.

94. The conjugate of claim 93, wherein W is selected from —P═O(OH)₂, —SO₃H, —CO₂H and —CH(CO₂H)₂, or a salt thereof.

95. The conjugate of any one of claims 92 to 94, wherein Z¹ is —(CH₂)ⱼ— and j is 1 to 3.

96. The conjugate of any one of claims 92 to 95, wherein Z¹ is —CH═CH—.

97. The conjugate of any one of claims 92 to 96, wherein Z² is O or S.

98. The conjugate of any one of claims 92 to 96, wherein Z² is —NR²¹—.

99. The conjugate of any one of claims 92 to 96, wherein Z² is —C(R²²)₂—.

100. The conjugate of any one of claims 92 to 94, wherein:
Z¹ is selected from —(CH₂)ᵣ—, substituted (C₁-C₃)alkylene and —CH═CH—;
j is 1 to 3; and
Z² is selected from O and CH₂.

101. The conjugate of claim 100, wherein:
Z¹ is —(CH₂)₂—, —CH₂—CF₂— or —CH₂—CHF—; and
Z² is O.

102. The conjugate of claim 100, wherein:
Z¹ is —(CH₂)₂—, —CH₂—CF₂— or —CH₂—CHF—; and
Z² is CH₂.

103. The conjugate of claim 100, wherein:
Z¹ is —CH═CH—; and
Z² is O.

104. The conjugate of claim 100, wherein:
Z¹ is —CH═CH—; and
Z² is CH₂.

105. The conjugate of any one of claims 92 to 104, wherein X is selected from:

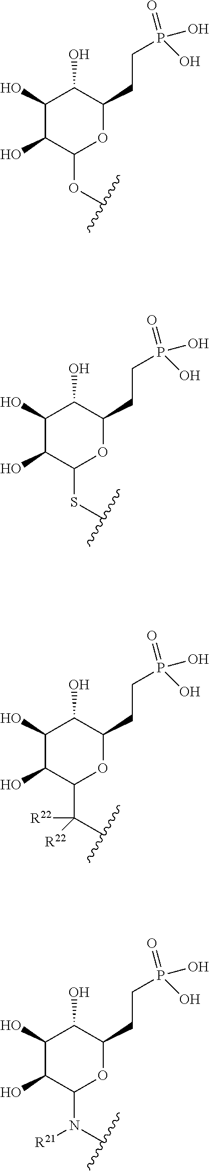

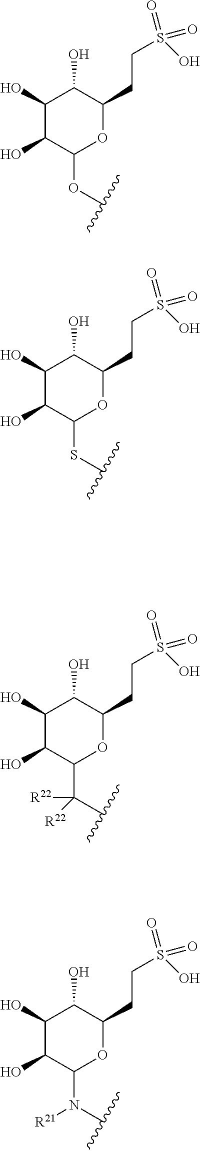

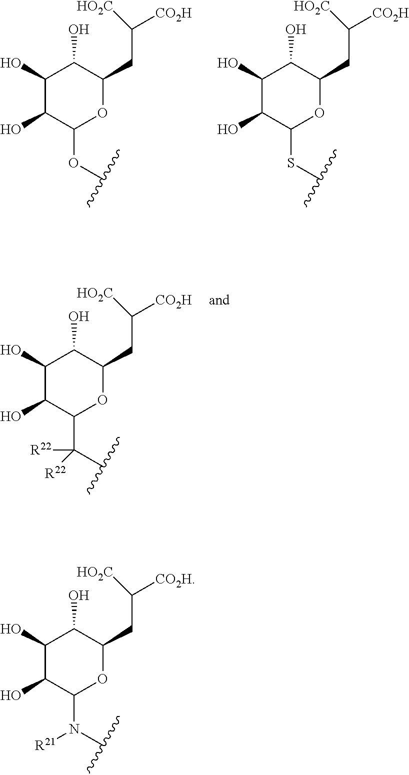

106. The conjugate of any one of claims 76 to 91, wherein X is a moiety that binds to ASGPR and is selected from formula (III-a) to (III-j): 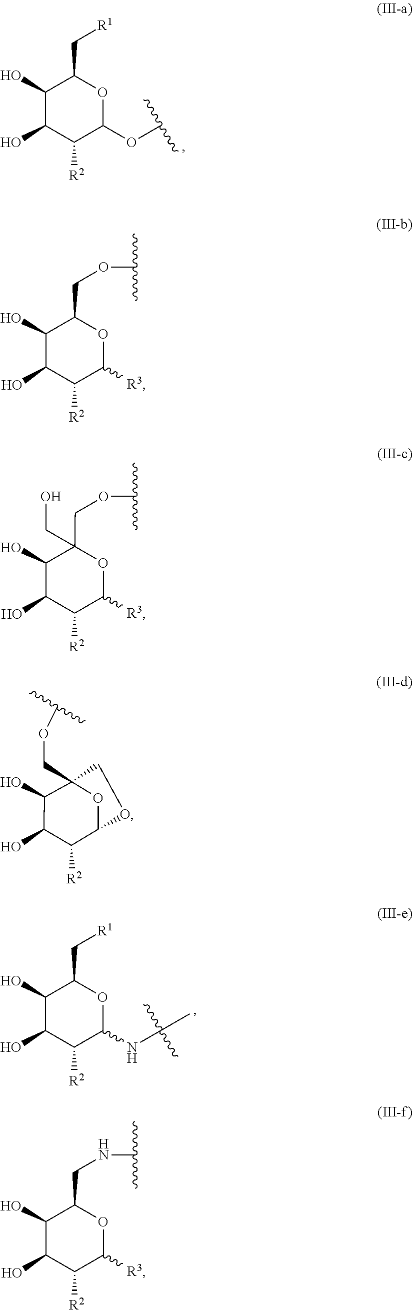 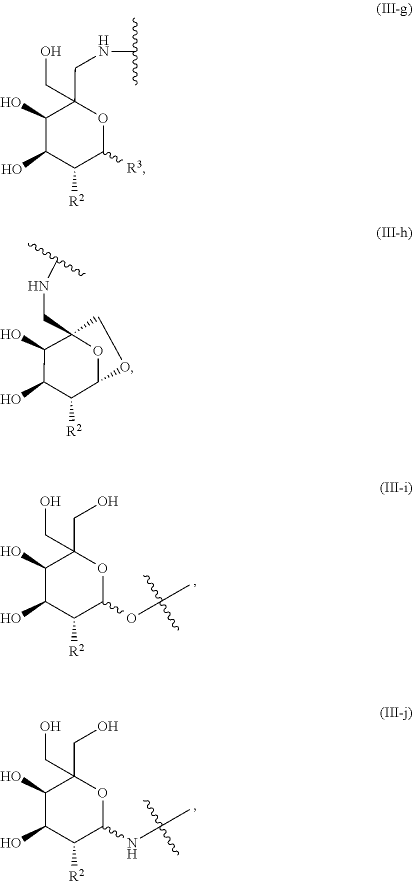
wherein:
R¹ is selected from —OH, —OC(O)R, and 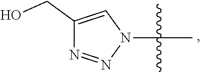
wherein R is C₁₋₆ alkyl;
R² is selected from —NHCOCH₃, —NHCOCF₃, —NHCOCH₂CF₃, —OH, and 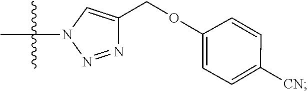 [SMILES: *n1cc(COc2ccc(C#N)cc2)nn1]
and
R³ is selected from —H, —OH, —CH₃, —OCH₃, and —OCH₂CH═CH₂.

107. The conjugate of claim 106, wherein X is:

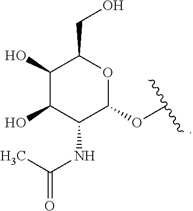 [SMILES: *O[C@H]1O[C@H](CO)[C@H](O)[C@H](O)[C@H]1NC(C)=O]

108. The conjugate of claim 106, wherein X is:

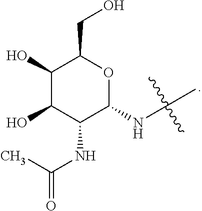 [SMILES: *N[C@H]1O[C@H](CO)[C@H](O)[C@H](O)[C@H]1NC(C)=O]

109. The conjugate of claims 76 to 108, wherein the linker L is of formula (IIa):
-[(L¹)ₐ-(L²)_{b}-(L³)_{c}]ₙ-(L⁴)_{d}-(L⁵)ₑ-(L⁶)_{f}-(L⁷)_{g}- (IIa)
wherein
each L¹ to L⁷ is independently a linking moiety and together provide a linear or branched linker between X and Y;
a is 1 or 2;
b, c, d, e, f, and g are each independently 0, 1, or 2;
n is 1 to 6 (e.g., n is 1 to 5, or 2 to 6, or 1, 2 or 3).

110. The conjugate of claim 109, wherein:
when d is 0, n is 1;
when d is 1, n is 1 to 3; and
when d is 2, n is 1 to 6.

111. The conjugate of claim 109 or 110, wherein -(L¹)ₐ- comprises an optionally substituted aryl or heteroaryl linking moiety.

112. The conjugate of claim 111, wherein each L¹ is independently selected from 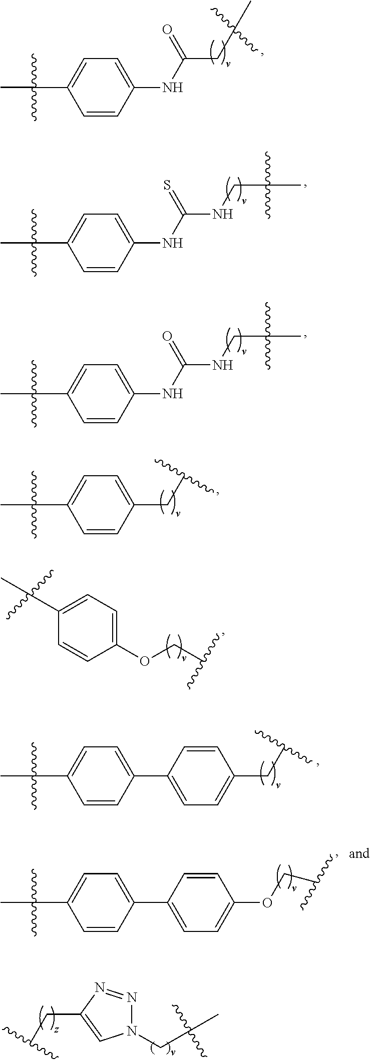
wherein v is 0 to 10 and z is 0 to 10.

113. The conjugate of any one of claims 109 to 112, wherein:
each L² is independently selected from —C₁₋₆-alkylene-, —NHCO—C₁₋₆-alkylene-, —CONH—C₁₋₆-alkylene-, —O(CH₂)ₚ—, and —(OCH₂CH₂)ₚ—, wherein p is 1 to 10; and
each L³ is independently selected from: 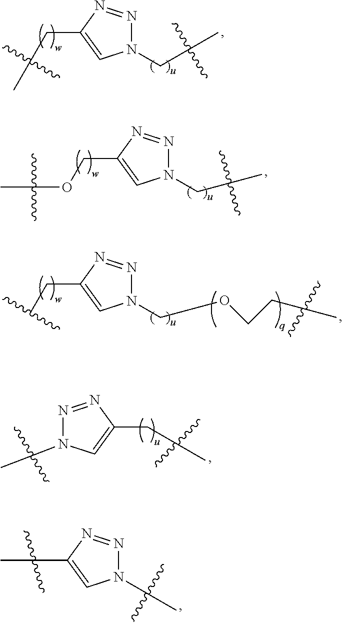
and —(OCH₂CH₂)_{q}—, wherein q is 1 to 10, u is 0 to 10, and w is 1 to 10.

114. The conjugate of any one of claims 109 to 113, wherein when n is 2 or more, at least one L⁴ is present and is a branched linking moiety.

115. The conjugate of any one of claims 109 to 114, wherein each L⁴ is independently selected from:
—OCH₂CH₂—, 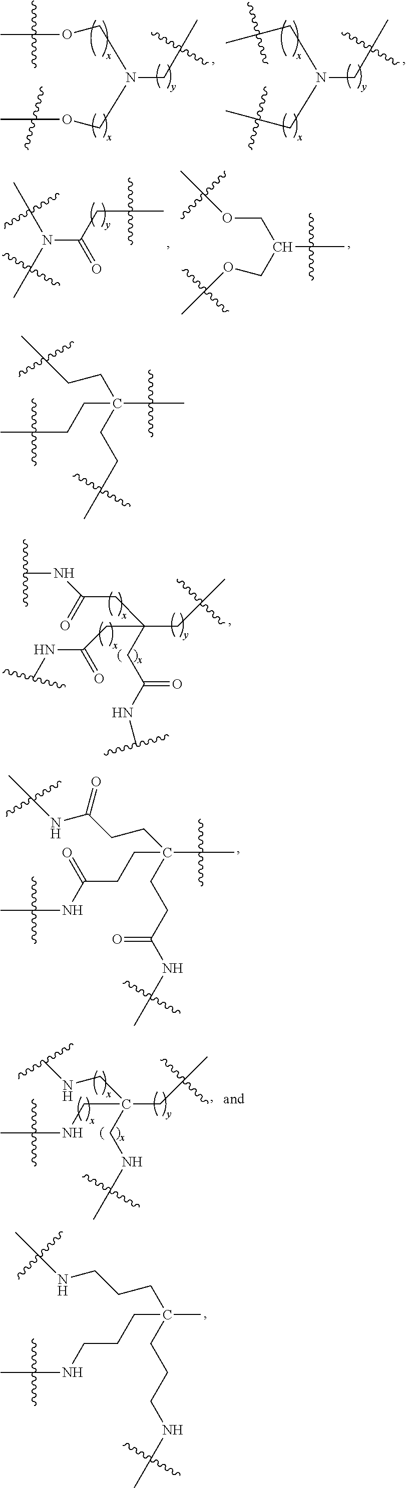
wherein each x and y are each independently 1 to 10.

116. The conjugate of any one of claims 109 to 115, wherein:
each L⁵ is independently —NHCO—C₁₋₆-alkylene-, —CONH—C₁₋₆-alkylene-, —C₁₋₆-alkylene-, 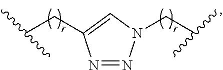
or —(OCH₂CH₂), —;
each L⁶ is independently —NHCO—C₁₋₆-alkylene-, —CONH—C₁₋₆-alkylene-, —C₁₋₆-alkylene-, or —(OCH₂CH₂)ₛ—;
each L⁷ is independently —NHCO—C₁₋₆-alkylene-, —CONH—C₁₋₆-alkylene-, —C₁₋₆-alkylene-, —(OCH₂CH₂)ₜ—, or —OCH₂—; and
r, s, and t are each independently 1 to 20.

117. The conjugate of any one of claims 109 to 116, wherein a is 1.

118. The conjugate of any one of claims 109 to 117, wherein at least one of b, c, e, f, and g is not 0.

119. The conjugate of any one of claims 109 to 118, wherein at least one of b or c is not 0 and at least one of e, f, and g is not 0.

120. The conjugate of any one of claims 109 to 119, wherein a, b, and c are each independently 1 or 2.

121. The conjugate of any one of claims 109 to 120, wherein the linker L is selected from any one of the structures of Tables 2-3.

122. The conjugate of claim 76 or 77, wherein the conjugate is selected from:
ii) a conjugate derived from conjugation of a compound of any one of the structures of Tables 5-9 and a biomolecule;
iii) a conjugate derived from conjugation of a compound of any one of the structures of Table 5-9 and a polypeptide; or
iv) a conjugate derived from conjugation of a compound of any one of the structures of Table 5-9 and an antibody or antibody fragment.

123. The conjugate of any one of claims 77-122, wherein the antibody or antibody fragment is an IgG antibody.

124. The conjugate of any one of claims 77-122, wherein the antibody or antibody fragment is a humanized antibody.

125. The conjugate of any one of claims 77-124, wherein the antibody or antibody fragment specifically binds to a secreted or soluble protein.

126. The conjugate of any one of claims 77-124, wherein the antibody or antibody fragment specifically binds to a cell surface receptor.

127. A method of internalizing a target protein in a cell comprising a cell surface receptor selected from M6PR and ASGPR, the method comprising:
contacting a cellular sample comprising the cell and the target protein with an effective amount of a compound according to any one of claims 1 to 75, or a conjugate according to any one of claims 76 to 132, wherein the compound or conjugate specifically binds the target protein and specifically binds the cell surface receptor to facilitate cellular uptake of the target protein.

128. The method of claim 127, wherein the target protein is a membrane bound protein.

129. The method of claim 127, wherein the target protein is an extracellular protein.

130. The method of any one of claims 127 to 129, wherein the compound or conjugate comprises an antibody or antibody fragment (Ab) that specifically binds the target protein.

131. A method of reducing levels of a target protein in a biological system, the method comprising:
contacting the biological system with an effective amount of a compound according to any one of claims 1 to 75, or a conjugate according to any one of claims 76 to 126, wherein the compound or conjugate specifically binds the target protein and specifically binds a cell surface receptor of cells in the biological system to facilitate cellular uptake and degradation of the target protein.

132. The method of claim 131, wherein the biological system comprises cells that comprise the cell surface receptor M6PR.

133. The method of claim 131, wherein the biological system comprises cells that comprise the cell surface receptor ASGPR.

134. The method of any one of claims 131 to 133, wherein the biological system is a human subject.

135. The method of any one of claims 131 to 133, wherein the biological system is an in vitro cellular sample.

136. The method of any one of claims 131 to 135, wherein the target protein is a membrane bound protein.

137. The method of any one of claims 137 to 135, wherein the target protein is an extracellular protein.

138. A method of treating a disease or disorder associated with a target protein, the method comprising:
administering to a subject in need thereof an effective amount of a compound according to any one of claims 1 to 75, or a conjugate according to any one of claims 76 to 126, wherein the compound or conjugate specifically binds the target protein.

139. The method of claim 138, wherein the disease or disorder is an inflammatory disease.

140. The method of claim 138, wherein the disease or disorder is an autoimmune disease.

141. The method of claim 138, wherein the disease or disorder is a cancer.


SEQUENCE LISTING (6 sequences)

SEQ ID NO: 1 - protein, 449 aa, Artificial Sequence
  Cetuximab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Cetuximab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 223 aa, Artificial Sequence
  Matuzumab heavy chain Fab
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 4 - protein, 212 aa, Artificial Sequence
  Matuzumab light chain
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 5 - protein, 448 aa, Artificial Sequence
  Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC